(19) United States
(12) Patent Application Publication
(10) Pub. No.: US 2014/0343049 A1
(43) Pub. Date: Nov. 20, 2014

(54) DIHYDROFURAN DERIVATIVES AS INSECTICIDAL COMPOUNDS

(71) Applicants: Julie Clementine Toueg, Stein (CH); Tomas Smejkal, Stein (CH); Sebastian Volker Wendeborn, Stein (CH); Jerome Yves Cassayre, Stein (CH); Myriem El Qacemi, Stein (CH); Bernhard Breit, Freiburg (DE); Lisa Diab, Al-chakdouf (LB); Regis Jean Georges Mondiere, Stein (CH); Nicolas Poirier, Stein (CH)

(72) Inventors: Julie Clementine Toueg, Stein (CH); Tomas Smejkal, Stein (CH); Sebastian Volker Wendeborn, Stein (CH); Jerome Yves Cassayre, Stein (CH); Myriem El Qacemi, Stein (CH); Bernhard Breit, Freiburg (DE); Lisa Diab, Al-chakdouf (LB); Regis Jean Georges Mondiere, Stein (CH); Nicolas Poirier, Stein (CH)

(73) Assignee: SYNGENTA PARTICIPATIONS AG, Basel (CH)

(21) Appl. No.: 14/239,373

(22) PCT Filed: Aug. 10, 2012
(86) PCT No.: PCT/EP2012/065753

§ 371 Date: Feb. 18, 2014

(30) Foreign Application Priority Data
Aug. 22, 2011 (EP) 11178221.5
Aug. 22, 2011 (EP) 11178224.9
Oct. 3, 2011 (EP) 11183687.0
Nov. 8, 2011 (EP) 11188276.7
Jul. 31, 2012 (EP) 12178614.9
Jul. 31, 2012 (EP) 12178615.6

(51) Int. Cl. C07D 417/12; A01N 43/08; C07D 409/12; A01N 43/20; C07D 405/10; A01N 43/84; A01N 43/78; C07D 413/12; A01N 43/80; C07D 405/12; A01N 43/50; A01N 43/52; C07D 307/28; A01N 43/653

(52) U.S. Cl. CPC C07D 417/12; C07D 307/28; A01N 43/08; C07D 409/12; A01N 43/20; C07D 405/10; A01N 43/653; A01N 43/78; C07D 413/12; A01N 43/80; C07D 405/12; A01N 43/50; A01N 43/52; A01N 43/84
USPC 514/231.5; 549/496; 514/471; 549/88; 514/430; 548/266.2; 514/383; 548/202; 514/365; 548/244; 514/380; 548/315.4; 514/397; 548/304.7; 514/394; 544/152

(57) ABSTRACT

The present invention relates to compounds of formula (IA) wherein Q^{A} is Q^{A}1 or Q^{A}2 P is P1, heterocycyl or heterocyclyl substituted by one to five Z; and wherein A¹, A², A³, A⁴, G¹, Z, R¹, R², R³ and R⁴ are as defined in claim 1; or a salt or N-oxide thereof. Furthermore, the present invention relates to processes and intermediates for preparing compounds of formula (IA), to insecticidal, acaricidal, nematicidal and molluscicidal compositions comprising the compounds of formula (IA) and to methods of using the compounds of formula (IA) to control insect, acarine, nematode and mollusc pests.

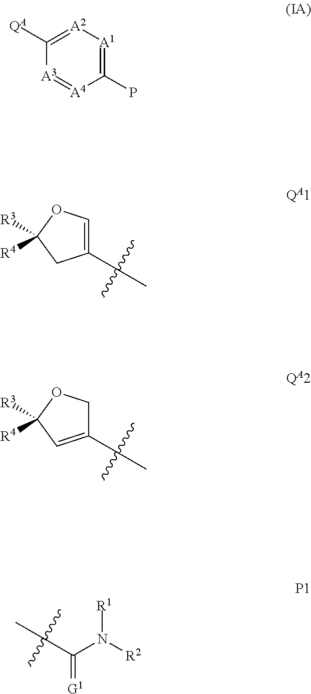

The present invention relates to certain dihydrofuran derivatives, to processes and intermediates for preparing these derivatives, to insecticidal, acaricidal, nematicidal and molluscicidal compositions comprising these derivatives and to methods of using these derivatives to control insect, acarine, nematode and mollusc pests. In particular, the invention relates to stereoisomers of said dihydrofuran derivatives.

Certain isoxazoline derivatives with insecticidal properties are disclosed, for example, in EP 1,731,512. However there is a continuing need to find new biologically active compounds as well as new biologically active compounds displaying superior properties for use as agrochemical active ingredients, for example greater biological activity, different spectrum of activity, increased safety profile, or increased biodegradability.

It has now surprisingly been found that certain dihydrofuran derivatives have insecticidal properties.

The present invention therefore provides compounds of formula (IA)

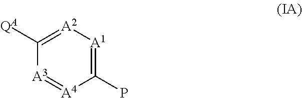

wherein

### Q^{A} is Q^{A}1 or Q^{A}2

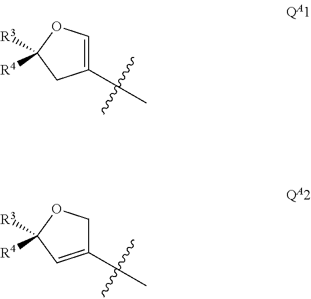

P is P1, heterocyclyl or heterocyclyl substituted by one to five Z;

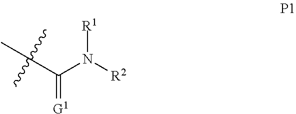

A¹, A², A³ and A⁴ are independently of each other C—H, C—R⁵, or nitrogen;
G¹ is oxygen or sulfur;
R¹ is hydrogen, C₁-C₈alkyl, C₁-C₈alkoxy-, C₁-C₈alkylcarbonyl-, or C₁-C₈alkoxycarbonyl-;
R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, C₁-C₆alkyl-O—N═CH—, C₁-C₆haloalkyl-O—N═CH—,
R³ is C₁-C₈haloalkyl;
R⁴ is aryl or aryl substituted by one to five R⁹, or heteroaryl or heteroaryl substituted by one to five R⁹;
each R⁵ is independently halogen, cyano, nitro, C₁-C₈alkyl, C₁-C₈haloalkyl, C₁-C₈alkenyl, C₁-C₈haloalkenyl, C₁-C₈alkynyl, C₁-C₈haloalkynyl, C₃-C₁₀cycloalkyl, C₁-C₈alkoxy-, C₁-C₈haloalkoxy-, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;
each R⁶ is independently halogen, cyano, nitro, hydroxy, amino, C₁-C₈alkylamino, (C₁-C₈alkyl)₂-amino, C₁-C₈alkylcarbonylamino, C₁-C₈haloalkylcarbonylamino, C₁-C₈alkoxy-, C₁-C₈haloalkoxy-, aryloxy or aryloxy substituted by one to five R¹⁰, aryloxy-C₁-C₄alkylene or aryloxy-C₁-C₄alkylene wherein the aryl moiety is substituted by one to five R¹⁰, C₁-C₈alkylcarbonyl-, C₁-C₈alkoxycarbonyl-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, aryl-C₁-C₄alkylthio or aryl-C₁-C₄alkylthio wherein the aryl moiety is substituted by one to five R¹⁰;
each R⁷ is independently halogen, C₁-C₈alkyl, C₁-C₈alkenyl, C₁-C₈alkynyl, C₁-C₈alkyl-O—N═, C₁-C₈haloalkyl-O—N═; C₁-C₈alkoxy, C₁-C₈akoxycarbonyl;
each R⁸ is independently halogen, cyano, nitro, oxo, C₁-C₈alkyl, C₁-C₈haloalkyl, C₁-C₈cyanoalkyl, C₂-C₈alkenyl, C₂-C₈haloalkenyl, C₂-C₈alkynyl, C₂-C₈haloalkynyl, C₃-C₁₀cycloalkyl, C₃-C₁₀cycloalkyl-C₁-C₄alkylene, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylaminosulfonyl, (C₁-C₈alkyl)₂-aminosulfonyl-, C₁-C₈alkylcarbonyl-, C₁-C₈alkoxycarbonyl-, aryl or aryl substituted by one to five R¹⁰, heterocyclyl or heterocyclyl substituted by one to five R¹⁰, aryl-C₁-C₄alkylene or aryl-C₁-C₄alkylene wherein the aryl moiety is substituted by one to five R¹⁰, heterocyclyl-C₁-C₄alkylene or heterocyclyl-C₁-C₄alkylene wherein the heterocyclyl moiety is substituted by one to five R¹⁰, aryloxy or aryloxy substituted by one to five R¹⁰, aryloxy-C₁-C₄alkylene or aryloxy-C₁-C₄alkylene wherein the aryl moiety is substituted by one to five R¹⁰;
each R⁹ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylcarbonyl-, C₁-C₈alkoxycarbonyl-, aryl or aryl substituted by one to five R¹⁰, or heterocyclyl or heterocyclyl substituted by one to five R¹⁰;
each R¹⁰ is independently halogen, cyano, nitro, C₁-C₄alkyl, C₁-C₄haloalkyl, C₁-C₄alkoxy-, or C₁-C₄haloalkoxy-;
each Z is independently halogen, C₁-C₁₂alkyl or C₁-C₁₂alkyl substituted by one to five R⁶, nitro, C₁-C₁₂alkoxy or C₁-C₁₂alkoxy substituted by one to five R⁶, cyano, C₁-C₁₂alkylsulfinyl, C₁-C₁₂alkylsulfonyl, C₁-C₁₂haloalkylsulfinyl, C₁-C₁₂haloalkylsulfonyl, hydroxyl or thiol; or a salt or N-oxide thereof.

The invention also provides mixtures comprising a compound of formula (IA) and a compound of formula (IB)

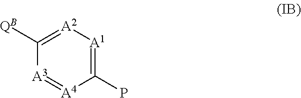

wherein Q^{B} is Q^{B}1 or Q^{B}2

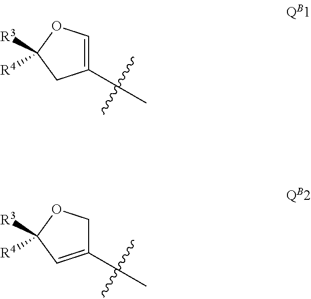

wherein A¹, A², A³, A⁴, P, R³ and R⁴ are as defined for compounds of formula (IA); and wherein the mixture is enriched for the compound of formula (IA).

The compounds of formula (IA) may exist in different geometric or optical isomers or tautomeric forms. Other than the chiral configuration at the asymmetric carbon indicated, this invention covers all such isomers and tautomers and mixtures thereof in all proportions as well as isotopic forms such as deuterated compounds. The invention also covers N-oxides and salts.

The compounds of the invention may contain one or more additional asymmetric carbon atoms and may exist as enantiomers (or as pairs of diastereoisomers) or as mixtures of such.

Alkyl groups (either alone or as part of a larger group, such as alkoxy-, alkylthio-, alkylsulfinyl-, alkylsulfonyl-, alkylcarbonyl- or alkoxycarbonyl-) can be in the form of a straight or branched chain and are, for example, methyl, ethyl, propyl, prop-2-yl, butyl, but-2-yl, 2-methyl-prop-1-yl or 2-methyl-prop-2-yl. The alkyl groups are preferably C₁-C₆, more preferably C₁-C₄, most preferably C₁-C₃ alkyl groups. Where an alkyl moiety is said to be substituted, the alkyl moiety is preferably substituted by one to four substituents, most preferably by one to three substituents.

Alkylene groups can be in the form of a straight or branched chain and are, for example, —CH₂—, —CH₂—CH₂—, —CH(CH₃)—, —CH₂—CH₂—CH₂—, —CH(CH₃)—CH₂—, or —CH(CH₂CH₃)—. The alkylene groups are preferably C₁-C₃, more preferably C₁-C₂, most preferably C₁ alkylene groups.

Alkenyl groups can be in the form of straight or branched chains, and can be, where appropriate, of either the (E)- or (Z)-configuration. Examples are vinyl and allyl. The alkenyl groups are preferably C₂-C₆, more preferably C₂-C₄, most preferably C₂-C₃ alkenyl groups.

Alkynyl groups can be in the form of straight or branched chains. Examples are ethynyl and propargyl. The alkynyl groups are preferably C₂-C₆, more preferably C₂-C₄, most preferably C₂-C₃ alkynyl groups.

Halogen is fluorine, chlorine, bromine or iodine.

Haloalkyl groups (either alone or as part of a larger group, such as haloalkoxy-, haloalkylthio-, haloalkylsulfinyl- or haloalkylsulfonyl-) are alkyl groups which are substituted by one or more of the same or different halogen atoms and are, for example, difluoromethyl, trifluoromethyl, chlorodifluoromethyl or 2,2,2-trifluoro-ethyl.

Haloalkenyl groups are alkenyl groups which are substituted by one or more of the same or different halogen atoms and are, for example, 2,2-difluoro-vinyl or 1,2-dichloro-2-fluoro-vinyl.

Haloalkynyl groups are alkynyl groups which are substituted by one or more of the same or different halogen atoms and are, for example, 1-chloro-prop-2-ynyl.

Cycloalkyl groups or carbocyclic rings can be in mono- or bi-cyclic form and are, for example, cyclopropyl, cyclobutyl, cyclohexyl and bicyclo[2.2.1]heptan-2-yl. The cycloalkyl groups are preferably C₃-C₈, more preferably C₃-C₆ cycloalkyl groups. Where a cycloalkyl moiety is said to be substituted, the cycloalkyl moiety is preferably substituted by one to four substituents, most preferably by one to three substituents.

Aryl groups (either alone or as part of a larger group, such as aryl-alkylene-) are aromatic ring systems which can be in mono-, bi- or tricyclic form. Examples of such rings include phenyl, naphthyl, anthracenyl, indenyl or phenanthrenyl. Preferred aryl groups are phenyl and naphthyl, phenyl being most preferred. Where an aryl moiety is said to be substituted, the aryl moiety is preferably substituted by one to four substituents, most preferably by one to three substituents.

Heteroaryl groups (either alone or as part of a larger group, such as heteroaryl-alkylene-) are aromatic ring systems containing at least one heteroatom and consisting either of a single ring or of two or more fused rings. Preferably, single rings will contain up to three heteroatoms and bicyclic systems up to four heteroatoms which will preferably be chosen from nitrogen, oxygen and sulfur. Examples of monocyclic groups include pyridyl, pyridazinyl, pyrimidinyl, pyrazinyl, pyrrolyl, pyrazolyl, imidazolyl, triazolyl (e.g. 1.2.4 triazoyl), furanyl, thiophenyl, oxazolyl, isoxazolyl, oxadiazolyl, thiazolyl, isothiazolyl and thiadiazolyl. Examples of bicyclic groups include purinyl, quinolinyl, cinnolinyl, quinoxalinyl, indolyl, indazolyl, benzimidazolyl, benzothiophenyl and benzothiazolyl. Monocyclic heteroaryl groups are preferred, pyridyl being most preferred. Where a heteroaryl moiety is said to be substituted, the heteroaryl moiety is preferably substituted by one to four substituents, most preferably by one to three substituents.

Heterocyclyl groups or heterocyclic rings (either alone or as part of a larger group, such as heterocyclyl-alkylene-) are defined to include heteroaryl groups and in addition their unsaturated or partially unsaturated analogues. Examples of monocyclic groups include isoxazolyl, thietanyl, pyrrolidinyl, tetrahydrofuranyl, [1,3]dioxolanyl, piperidinyl, piperazinyl, [1,4]dioxanyl, and morpholinyl or their oxidised versions such as 1-oxo-thietanyl and 1,1-dioxo-thietanyl. Examples of bicyclic groups include 2,3-dihydro-benzofuranyl, benzo[1,4]dioxolanyl, benzo[1,3]dioxolanyl, chromenyl, and 2,3-dihydro-benzo[1,4]dioxinyl. Where a heterocyclyl moiety is said to be substituted, the heterocyclyl moiety is preferably substituted by one to four substituents, most preferably by one to three substituents.

Preferred values of P, A¹, A², A³, A⁴, G¹, Z, R¹, R², R³, R⁴, R⁵, R⁶, R⁷, R⁸, R⁹ and R¹⁰ are, in any combination, as set out below.

Preferably, P is P1, or a heterocycle selected from H1 to H9

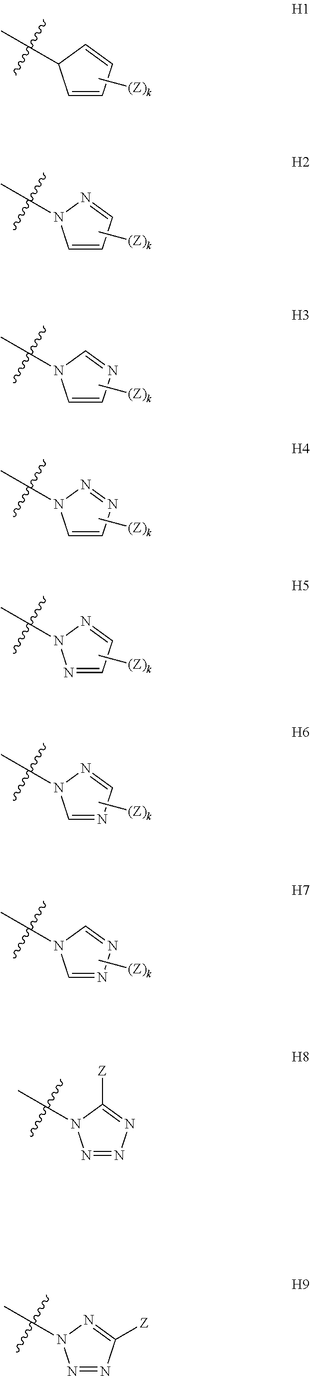

k is 0, 1 or 2.

Preferably no more than two of A¹, A², A³ and A⁴ are nitrogen, more preferably no more than one of A¹, A², A³ and A⁴ is nitrogen.

Preferably A¹ is C—H or C—R⁵, most preferably A¹ is C—R⁵.

Preferably A² is C—H or C—R⁵, most preferably A² is C—H.

Preferably A³ is C—H or C—R⁵, most preferably A³ is C—H.

Preferably A⁴ is C—H or C—R⁵, most preferably A⁴ is C—H.

In one preferred group of compounds A¹ is C—R⁵, A² is C—H, A³ is C—H or nitrogen and A⁴ is C—H or nitrogen.

In another preferred group of compounds A¹ is C—R⁵, A² is C—H, A³ is C—H and A⁴ is C—H.

Preferably G¹ is oxygen.

Preferably R¹ is hydrogen, methyl, ethyl, methylcarbonyl-, or methoxycarbonyl-, more preferably hydrogen, methyl or ethyl, most preferably hydrogen.

Preferably, R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A

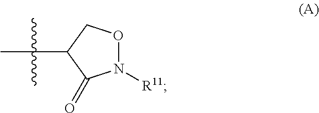

wherein R¹¹ is hydrogen, methyl, ethyl, propyl, isopropyl, butyl, cyclopropyl, cyclopropyl-methyl, cyclobutyl, cyclobutyl-methyl, oxetanyl, thietanyl, trifluoroethyl, difluoroethyl, allyl, propargyl, cyanomethyl, benzyl, benzyl substituted by one to three R¹², or R¹¹ is pyridyl-methyl- or pyridyl-methyl-substituted by one to three R¹²; and each R¹² is independently fluoro, chloro, bromo, trifluoromethyl, trifluoromethoxy, cyano or methoxy.

More preferably R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A

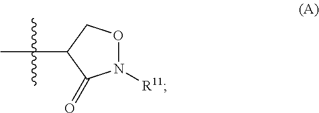

wherein each aryl group is a phenyl group and each heterocyclyl group is independently selected from pyridyl, pyridazinyl, pyrimidinyl, pyrazinyl, pyrrolyl, pyrazolyl, imidazolyl, triazolyl, tetrahydrothiophenyl, tetrazolyl, furanyl, thiophenyl, oxazolyl, isoxazolyl, oxadiazolyl, thiazolyl, isothiazolyl, thiadiazolyl, quinolinyl, cinnolinyl, quinoxalinyl, indolyl, indazolyl, benzimidazolyl, benzothiophenyl, benzothiazolyl, oxetanyl, thietanyl, oxo-thietanyl, dioxo-thietanyl, pyrrolidinyl, tetrahydrofuranyl, [1,3]dioxolanyl, piperidinyl, piperazinyl, [1,4]dioxanyl, and morpholinyl, 2,3-dihydro-benzofuranyl, benzo[1,3]dioxolanyl, and 2,3-dihydro-benzo[1,4]dioxinyl.

More preferably R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A

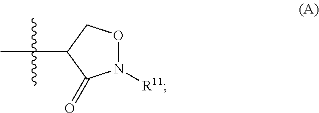

wherein each aryl group is a phenyl group and each heterocyclyl group is selected from 1,2,3 triazolyl, 1,2,4 triazolyl, tetrazolyl, pyrimidinyl, pyrazinyl, pyridazinyl, tetrahydrothiophenyl, isoxazolinyl, pyridyl, tetrahydrofuranyl, imidazolyl, pyrazolyl, pyrrolyl, thiazolyl, oxetanyl, thietanyl, oxo-thietanyl and dioxo-thietanyl.

More preferably still R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A

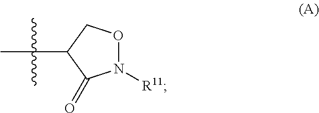

wherein each aryl group is a phenyl group and each heterocyclyl group is selected from pyridyl, tetrahydrofuranyl, imidazolyl, pyrazolyl, pyrrolyl, thiazolyl, oxetanyl, thietanyl, oxo-thietanyl and dioxo-thietanyl.

More preferably still R² is C₁-C₈alkyl or C₁-C₈alkyl substituted by one to five R⁶, C₃-C₁₀cycloalkyl or C₃-C₁₀cycloalkyl substituted by one to five R⁷, phenyl-C₁-C₄alkylene- or phenyl-C₁-C₄alkylene- wherein the phenyl moiety is substituted by one to five R⁸, pyridyl-C₁-C₄alkylene- or pyridyl-C₁-C₄alkylene- wherein the pyridyl moiety is substituted by one to four R⁸, tetrahydrofuranyl-C₁-C₄alkylene- or tetrahydrofuranyl-C₁-C₄alkylene- wherein the tetrahydrofuranyl moiety is substituted by one to five R⁸, imidazolyl-C₁-C₄alkylene- or imidazolyl-C₁-C₄alkylene- wherein the imidazolyl moiety is substituted by one to three R⁸, pyrazolyl-C₁-C₄alkylene- or pyryazolyl-C₁-C₄alkylene- wherein the pyrazolyl moiety is substituted by one to three R⁸, pyrrolyl-C₁-C₄alkylene- or pyrrolyl-C₁-C₄alkylene- wherein the pyrrolyl moiety is substituted by one to four R⁸, thiazolyl-C₁-C₄alkylene- or thiazolyl-C₁-C₄alkylene- wherein the thiazolyl moiety is substituted by one to four R⁸, oxetanyl-C₁-C₄ alkylene or oxetanyl-C₁-C₄ alkylene wherein the oxetanyl moiety is substituted by one to five R⁸, thietanyl-C₁-C₄ alkylene or thietanyl-C₁-C₄ alkylene wherein the thietanyl moiety is substituted by one to five R⁸, oxo-thietanyl-C₁-C₄ alkylene or oxo-thietanyl-C₁-C₄ alkylene wherein the oxo-thietanyl moiety is substituted by one to five R⁸, dioxo-thietanyl-C₁-C₄ alkylene or dioxo-thietanyl-C₁-C₄ alkylene wherein the dioxo-thietanyl moiety is substituted by one to five R⁸, oxetanyl or oxetanyl substituted by one to five R⁸, thietanyl or thietanyl substituted by one to five R⁸, oxo-thietanyl or oxo-thietanyl substituted by one to five R⁸, dioxo-thietanyl or dioxo-thietanyl substituted by one to five R⁸, C₁-C₈alkylaminocarbonyl-C₁-C₈ alkylene, C₁-C₈haloalkylaminocarbonyl-C₁-C₄ alkylene, C₃-C₈cycloalkyl-aminocarbonyl-C₁-C₄ alkylene, or group A

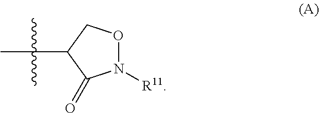

Even more preferably R² is C₁-C₈alkyl or C₁-C₈alkyl substituted by one to five R⁶, C₃-C₁₀cycloalkyl or C₃-C₁₀cycloalkyl substituted by one to five R⁷, phenyl-C₁-C₄alkylene- or phenyl-C₁-C₄alkylene- wherein the phenyl moiety is substituted by one to five R⁸, pyridyl-C₁-C₄alkylene- or pyridyl-C₁-C₄alkylene- wherein the pyridyl moiety is substituted by one to four R⁸, oxetanyl or oxetanyl substituted by one to five R⁸, thietanyl-C₁-C₄ alkylene or thietanyl-C₁-C₄ alkylene wherein the thietanyl moiety is substituted by one to five R⁸, oxo-thietanyl-C₁-C₄ alkylene or oxo-thietanyl-C₁-C₄ alkylene wherein the oxo-thietanyl moiety is substituted by one to five R⁸, dioxo-thietanyl-C₁-C₄ alkylene or dioxo-thietanyl-C₁-C₄ alkylene wherein the dioxo-thietanyl moiety is substituted by one to five R⁸, thietanyl or thietanyl substituted by one to five R⁸, oxo-thietanyl or oxo-thietanyl substituted by one to five R⁸, dioxo-thietanyl or dioxo-thietanyl substituted by one to five R⁸, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene, or group A

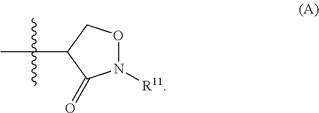

Yet even more preferably R² is C₁-C₈alkyl or C₁-C₈alkyl substituted by one to three halogen atoms, C₃-C₁₀cycloalkyl or C₃-C₁₀cycloalkyl substituted by one or two groups independently selected from fluoro and methyl, phenyl-C₁-C₄alkylene- or phenyl-C₁-C₄alkylene- wherein the phenyl moiety is substituted by one to five R⁸, pyridyl-C₁-C₄alkylene- or pyridyl-C₁-C₄alkylene- wherein the pyridyl moiety is substituted by one to four R⁸, thietanyl, oxo-thietanyl, dioxo-thietanyl, C₁-C₈alkylaminocarbonyl-methylene, C₁-C₈haloalkylaminocarbonyl-methylene, C₃-C₈cycloalkyl-aminocarbonyl-methylene, or group A

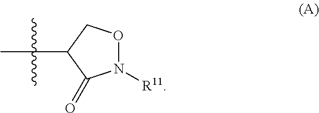

A group of preferred compounds are those wherein R² is C₁-C₆alkyl or C₁-C₆alkyl substituted by one to five R⁶, for example ethyl-, butyl-, but-2-yl-, 3-bromo-propyl-, 2,2,2-trifluoro-ethyl-, 3,3,3-trifluoro-propyl-, 2-methoxy-ethyl-, and 1-methoxy-prop-2-yl-.

A group of preferred compounds are those wherein R² is C₃-C₈cycloalkyl or C₃-C₈cycloalkyl substituted by one to five R⁷, for example cyclobutyl-, 2-fluoro-cyclopropyl and 2-methyl-cyclohex-1-yl-.

A group of preferred compounds are those wherein R² is aryl-C₁-C₂alkylene- or aryl-C₁-C₂alkylene- wherein the aryl moiety is substituted by one to five R⁸, for example phenyl-methyl-, 1-phenyl-eth-1-yl-, 2-phenyl-eth-1-yl-, (3-chloro-phenyl)-methyl-, (2-fluoro-phenyl)-methyl-, (4-methoxy-phenyl)-methyl-, (2-trifluoromethyl-phenyl)-methyl-, and (2-trifluoromethoxy-phenyl)-methyl-.

A group of preferred compounds are those wherein R² is heterocyclyl-C₁-C₂alkylene- or heterocyclyl-C₁-C₂alkylene- wherein the heterocyclyl moiety is substituted by one to five R⁸, for example (pyrid-2-yl)-methyl-, (pyrid-3-yl)-methyl-, (2-chloro-pyrid-5-yl)-methyl-, (1-methyl-1H-imidazol-4-yl)-methyl-, (furan-2-yl)-methyl-, 2-(thiophen-2′-yl)-eth-1-yl-, 2-(indol-3′-yl)-eth-1-yl-, (1H-benzimidazol-2-yl)-methyl-, (oxetan-2-yl)-methyl-, (tetrahydrofuran-2-yl)-methyl-, 2-([1′,3′]dioxolan-2′-yl)-eth-1-yl-, 2-(morpholin-4′-yl)-eth-1-yl-, 2-(benzo[1′,3′]dioxol-5′-yl)-eth-1-yl-, (2,3-dihydro-benzo[1,4]dioxin-6-yl)-methyl-, thietan-2-yl-methyl-, 1-oxo-thietan-2-yl-methyl-, 1,1-dioxo-thietan-2-yl-methyl-, thietan-3-yl-methyl-, 1-oxo-thietan-3-yl-methyl-, 1,1-dioxo-thietan-3-yl-methyl-, thietan-3-yl-ethyl-, 1-oxo-thietan-3-yl-ethyl-, and 1,1-dioxo-thietan-3-yl-ethyl-, preferably thietan-2-yl-methyl-, 1-oxo-thietan-2-yl-methyl-, 1,1-dioxo-thietan-2-yl-methyl-, thietane-3-yl-methyl-, 1-oxo-thietan-3-yl-methyl-, 1,1-dioxo-thietan-3-yl-methyl-, thietan-3-yl-ethyl-, 1-oxo-thietan-3-yl-ethyl-, and 1,1-dioxo-thietan-3-yl-ethyl-.

A group of preferred compounds are those wherein R² is heterocyclyl-C₁-C₂alkylene- or heterocyclyl-C₁-C₂alkylene- wherein the heterocyclyl moiety is substituted by one to five R⁸ in which the heterocyclyl group is selected from 1,2,3 triazolyl, 1,2,4 triazolyl, tetrazolyl, pyrimidinyl, pyrazinyl, pyridazinyl, tetrahydrothiophenyl, isoxazolinyl, pyridyl, tetrahydrofuranyl, imidazolyl, pyrazolyl, pyrrolyl, thiazolyl, oxetanyl, thietanyl, oxo-thietanyl and dioxo-thietanyl, preferably the heterocyclyl group is selected from thietanyl, oxo-thietanyl and dioxo-thietanyl.

A group of preferred compounds are those wherein R² is heteroaryl-C₁-C₂alkylene- or heteroaryl-C₁-C₂alkylene- wherein the heteroaryl moiety is substituted by one to five R⁸.

A group of preferred compounds are those wherein R² is aryl or aryl substituted by one to five R⁸, for example 2-chloro-phenyl-, 3-fluoro-phenyl-, 2-methyl-phenyl-, 2-chloro-6-methyl-phenyl-, 2-trifluoromethyl-phenyl-, and 2,4-dimethoxy-phenyl-.

A group of preferred compounds are those wherein R² is heterocyclyl or heterocyclyl substituted by one to five R⁸, for example 3-methyl-pyrid-2-yl-, 1,3-dimethyl-1H-pyrazol-5-yl-, 4-methyl-thiazol-2-yl-, 5-methyl-thiadiazol-2-yl-, quinolin-2-yl-, quinolin-5-yl-, benzothiazol-6-yl-, 4-methyl-benzothiazol-2-yl-, thietan-3-yl-, 1-oxo-thietan-3-yl-, 1,1-dioxo-thietan-3-yl-, and 3-methyl-thietan-3-yl-, more preferably R² is oxetanyl, thietanyl, oxo-thietanyl or dioxo-thietanyl each optionally substituted by one to five R⁸, most preferably R² is thietanyl, oxo-thietanyl or dioxo-thietanyl each optionally substituted by one to five R⁸. It is particularly preferred that the oxetanyl, thietanyl, oxo-thietanyl or dioxo-thietanyl ring is linked via the 3-position.

A group of preferred compounds are those wherein R² is heterocyclyl- or heterocyclyl substituted by one to five R⁸ in which the heterocyclyl group is selected from 1,2,3 triazolyl, 1,2,4 triazolyl, tetrazolyl, pyrimidinyl, pyrazinyl, pyridazinyl, tetrahydrothiophenyl, isoxazolinyl, pyridyl, tetrahydrofuranyl, imidazolyl, pyrazolyl, pyrrolyl, thiazolyl, oxetanyl, thietanyl, oxo-thietanyl and dioxo-thietanyl, preferably the heterocyclyl group is selected from thietanyl, oxo-thietanyl and dioxo-thietanyl.

A group of preferred compounds are those wherein R² is C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene, more preferably C₁-C₄alkylaminocarbonyl-C₁-C₄ alkylene, C₁-C₄haloalkylaminocarbonyl-C₁-C₄ alkylene, or C₃-C₆cycloalkyl-aminocarbonyl-C₁-C₄ alkylene, most preferably C₁-C₄alkylaminocarbonyl-C₁-C₂ alkylene or C₁-C₄haloalkylaminocarbonyl-C₁-C₂ alkylene.

A group of preferred compounds are those wherein R² is group A

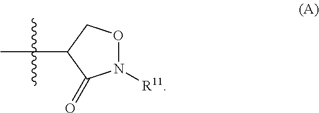

Preferably R³ is chlorodifluoromethyl or trifluoromethyl, most preferably trifluoromethyl.

Preferably R⁴ is aryl or aryl substituted by one to five R⁹, more preferably aryl substituted by one to three R⁹, more preferably phenyl substituted by one to three R⁹, even more preferably 3,5-dibromo-phenyl-, 3,5-dichloro-phenyl-, 3,5-bis-(trifluoromethyl)-phenyl-, 3,4-dichloro-phenyl-, 3,4,5-trichloro-phenyl-3-trifluoromethyl-phenyl-, 4-bromo-3,5-dichloro-phenyl or 3-chloro-5-trifluoromethyl-phenyl, most preferably R⁴ is 3,5-dichloro-phenyl.

Preferably each R⁵ is independently halogen, cyano, nitro, C₁-C₈alkyl, C₁-C₈haloalkyl, C₁-C₈alkenyl, C₁-C₈haloalkenyl, C₁-C₈alkoxy-, or C₁-C₈haloalkoxy-, more preferably bromo, chloro, fluoro, cyano, nitro, methyl, ethyl, trifluoromethyl, vinyl, methoxy, difluoromethoxy, or trifluoromethoxy, most preferably methyl.

Preferably each R⁶ is independently halogen, cyano, nitro, hydroxy, C₁-C₈alkoxy-, C₁-C₈haloalkoxy-, C₁-C₈alkylcarbonyl-, C₁-C₈alkoxycarbonyl-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-, more preferably each R⁶ is independently halogen, cyano, nitro, hydroxy, C₁-C₈alkoxy-, C₁-C₈haloalkoxy-, mercapto, C₁-C₈alkylthio-, C₁-C₈haloalkylthio-, more preferably bromo, chloro, fluoro, methoxy, or methylthio, most preferably chloro, fluoro, or methoxy.

Preferably each R⁷ is independently halogen or C₁-C₈alkyl, more preferably each R⁷ is independently chloro, fluoro or methyl, most preferably fluoro or methyl.

Preferably each R⁸ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylcarbonyl-, C₁-C₈alkoxycarbonyl-, aryl or aryl substituted by one to five R¹⁰, or heterocyclyl or heterocyclyl substituted by one to five R¹⁰, more preferably each R⁸ is independently halogen, cyano, nitro, C₁-C₈alkyl, C₁-C₈haloalkyl, C₁-C₈alkoxy-, C₁-C₈haloalkoxy-, more preferably bromo, chloro, fluoro, cyano, nitro, methyl, ethyl, trifluoromethyl, methoxy, difluoromethoxy, or trifluoromethoxy, most preferably bromo, chloro, fluoro, cyano or methyl.

Preferably each R⁹ is independently halogen, C₁-C₈alkyl, C₁-C₈haloalkyl, C₁-C₈alkoxy-, C₁-C₈haloalkoxy-, C₁-C₈alkylthio-, or C₁-C₈haloalkylthio-, more preferably bromo, chloro, fluoro, trifluoromethyl, methoxy, or methylthio, most preferably bromo or chloro.

Preferably each R¹⁰ is independently bromo, chloro, fluoro, cyano, nitro, methyl, ethyl, trifluoromethyl, methoxy, difluoromethoxy, or trifluoromethoxy, more preferably bromo, chloro, fluoro, nitro, or methyl, most preferably chloro, fluoro, or methyl.

Preferably R¹¹ is methyl, ethyl or trifluoroethyl.

Preferably each Z is independently halogen, cyano, C₁-C₄alkyl, C₁-C₄haloalkyl, C₁-C₄alkoxy, or C₁-C₄haloalkoxy, more preferably each Z is independently hydrogen, halogen, methyl, halomethyl, methoxy or halomethoxy.

In one group of compounds of formula (IA)

### P is P1;

### Q is Q1 or Q2;

A¹, A², A³ and A⁴ are independently of each other C—H, C—R⁵, or nitrogen;
G¹ is oxygen or sulfur;
R¹ is hydrogen, C₁-C₈alkyl, C₁-C₈alkoxy-, C₁-C₈alkylcarbonyl-, or C₁-C₈alkoxycarbonyl-;
R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A

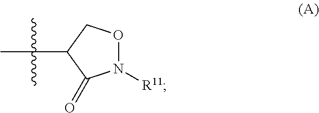

R³ is C₁-C₈haloalkyl;
R⁴ is aryl or aryl substituted by one to five R⁹, or heteroaryl or heteroaryl substituted by one to five R⁹;
each R⁵ is independently halogen, cyano, nitro, C₁-C₈alkyl, C₁-C₈haloalkyl, C₁-C₈alkenyl, C₁-C₈haloalkenyl, C₁-C₈alkynyl, C₁-C₈haloalkynyl, C₃-C₁₀cycloalkyl, C₁-C₈alkoxy-, C₁-C₈haloalkoxy-, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;
each R⁶ is independently halogen, cyano, nitro, hydroxy, C₁-C₈alkoxy-, C₁-C₈haloalkoxy-, C₁-C₈alkylcarbonyl-, C₁-C₈alkoxycarbonyl-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;
each R⁷ is independently halogen or C₁-C₈alkyl;
each R⁸ and R⁹ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylcarbonyl-, C₁-C₈alkoxycarbonyl-, aryl or aryl substituted by one to five R¹⁰, or heterocyclyl or heterocyclyl substituted by one to five R¹⁰;
each R¹⁰ is independently halogen, cyano, nitro, C₁-C₄alkyl, C₁-C₄haloalkyl, C₁-C₄alkoxy-, or C₁-C₄haloalkoxy-;
R¹¹ is hydrogen, methyl, ethyl, propyl, isopropyl, butyl, cyclopropyl, cyclopropyl-methyl, cyclobutyl, cyclobutyl-methyl, oxetanyl, thietanyl, trifluoroethyl, difluoroethyl, allyl, propargyl, cyanomethyl, benzyl, benzyl substituted by one to three R¹², or R¹¹ is pyridyl-methyl- or pyridyl-methyl- substituted by one to three R¹²;
each R¹² is independently fluoro, chloro, bromo, trifluoromethyl, trifluoromethoxy, cyano or methoxy; or
a salt or N-oxide thereof.

A group of preferred compounds are those wherein

A¹ is C—R⁵, A² is C—H, A³ is C—H or nitrogen and A⁴ is C—H or nitrogen;

G¹ is oxygen;

R¹ is hydrogen, methyl, ethyl, methylcarbonyl-, or methoxycarbonyl-;

R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A

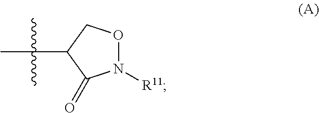

wherein each aryl group is a phenyl group and each heterocyclyl group is selected from pyridyl, tetrahydrofuranyl, imidazolyl, pyrazolyl, pyrrolyl, thiazolyl, oxetanyl, thietanyl, oxo-thietanyl and dioxo-thietanyl;

R³ is C₁-C₈ haloalkyl;

R⁴ is phenyl substituted by one to three R⁹;

R⁵ is independently halogen, cyano, nitro, C₁-C₈alkyl, C₁-C₈haloalkyl, C₁-C₈alkenyl, C₁-C₈haloalkenyl, C₁-C₈alkoxy-, or C₁-C₈haloalkoxy-;

each R⁶ is independently halogen, cyano, nitro, hydroxy, C₁-C₈alkoxy-, C₁-C₈haloalkoxy-, mercapto, C₁-C₈alkylthio-, C₁-C₈haloalkylthio-;

each R⁷ is independently chloro, fluoro or methyl;

each R⁸ is independently halogen, cyano, nitro, C₁-C₈alkyl, C₁-C₈haloalkyl, C₁-C₈alkoxy-, C₁-C₈haloalkoxy-;

each R⁹ is independently halogen, C₁-C₈alkyl, C₁-C₈haloalkyl, C₁-C₈alkoxy-, C₁-C₈haloalkoxy-, C₁-C₈alkylthio-, or C₁-C₈haloalkylthio-;

Another group of preferred compounds are those wherein

A¹ is C—R⁵, A² is C—H, A³ is C—H and A⁴ is C—H;

G¹ is oxygen;

R¹ is hydrogen, methyl or ethyl;

R² is C₁-C₈alkyl or C₁-C₈alkyl substituted by one to five R⁶, C₃-C₁₀cycloalkyl or C₃-C₁₀cycloalkyl substituted by one to five R⁷, phenyl-C₁-C₄alkylene- or phenyl-C₁-C₄alkylene- wherein the phenyl moiety is substituted by one to five R⁸, pyridyl-C₁-C₄alkylene- or pyridyl-C₁-C₄alkylene- wherein the pyridyl moiety is substituted by one to four R⁸, oxetanyl or oxetanyl substituted by one to five R⁸, thietanyl-C₁-C₄ alkylene or thietanyl-C₁-C₄ alkylene wherein the thietanyl moiety is substituted by one to five R⁸, oxo-thietanyl-C₁-C₄ alkylene or oxo-thietanyl-C₁-C₄ alkylene wherein the oxo-thietanyl moiety is substituted by one to five R⁸, dioxo-thietanyl-C₁-C₄ alkylene or dioxo-thietanyl-C₁-C₄ alkylene wherein the dioxo-thietanyl moiety is substituted by one to five R⁸, thietanyl or thietanyl substituted by one to five R⁸, oxo-thietanyl or oxo-thietanyl substituted by one to five R⁸, dioxo-thietanyl or dioxo-thietanyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A

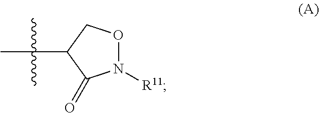

R³ is chlorodifluoromethyl or trifluoromethyl;

R⁴ is 3,5-dibromo-phenyl-, 3,5-dichloro-phenyl-, 3,5-bis-(trifluoromethyl)-phenyl-, 3,4-dichloro-phenyl-, 3,4,5-trichloro-phenyl-, 3-trifluoromethyl-phenyl-, 4-bromo-3,5-dichloro-phenyl or 3-chloro-5-trifluoromethyl-phenyl;

R⁵ is independently bromo, chloro, fluoro, cyano, nitro, methyl, ethyl, trifluoromethyl, vinyl, methoxy, difluoromethoxy, or trifluoromethoxy;

each R⁶ is independently bromo, chloro, fluoro, methoxy, or methylthio;

each R⁷ is independently fluoro or methyl;

each R⁸ is independently bromo, chloro, fluoro, cyano, nitro, methyl, ethyl, trifluoromethyl, methoxy, difluoromethoxy, or trifluoromethoxy;

R¹¹ is hydrogen, methyl, ethyl, propyl, isopropyl, butyl, cyclopropyl, cyclopropyl-methyl, cyclobutyl, cyclobutyl-methyl, oxetanyl, thietanyl, trifluoroethyl, difluoroethyl, allyl, propargyl, cyanomethyl, benzyl, benzyl substituted by one to three R¹², or R¹¹ is pyridyl-methyl- or pyridyl-methyl-substituted by one to three R¹²;

each R¹² is independently fluoro, chloro, bromo, trifluoromethyl, trifluoromethoxy, cyano or methoxy.

A further group of preferred compounds are those wherein

P is P1;

A¹ is C—R⁵, A² is C—H, A³ is C—H and A⁴ is C—H;

G¹ is oxygen;

R¹ is hydrogen;

R² is C₁-C₈alkyl or C₁-C₈alkyl substituted by one to three halogen atoms, C₃-C₁₀cycloalkyl or C₃-C₁₀cycloalkyl substituted by one or two groups independently selected from fluoro and methyl, phenyl-C₁-C₄alkylene- or phenyl-C₁-C₄alkylene- wherein the phenyl moiety is substituted by one to five R⁸, pyridyl-C₁-C₄alkylene- or pyridyl-C₁-C₄alkylene- wherein the pyridyl moiety is substituted by one to four R⁸, thietanyl, oxo-thietanyl, dioxo-thietanyl, C₁-C₄alkylaminocarbonyl-C₁-C₄ alkylene, C₁-C₄haloalkylaminocarbonyl-C₁-C₄ alkylene, C₃-C₆cycloalkyl-aminocarbonyl-C₁-C₄ alkylene, or group A

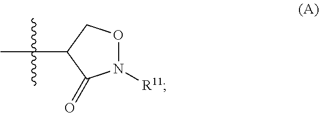

R³ is trifluoromethyl;

R⁴ is 3,5-dichloro-phenyl;

R⁵ is methyl;

each R⁸ is independently bromo, chloro, fluoro, cyano or methyl;

R¹¹ is methyl, ethyl or trifluoroethyl.

A further group of preferred compounds are those wherein

P is P1;

A¹ is C—R⁵, A² is CH, A³ is CH and A⁴ is CH;

G¹ is oxygen;

R¹ is hydrogen;

R² is C₂-C₆alkyl or C₂-C₆alkyl substituted by one to three halogen atoms, C₃-C₈cycloalkyl or C₃-C₈cycloalkyl substituted by one or two groups independently selected from fluoro and methyl, phenyl-C₁-C₂alkylene- or phenyl-C₁-C₂alkylene- wherein the phenyl moiety is substituted by one to five R⁸, pyridyl-C₁-C₂alkylene- or pyridyl-C₁-C₂alkylene- wherein the pyridyl moiety is substituted by one to four R⁸, thietanyl, oxo-thietanyl, dioxo-thietanyl, C₁-C₄alkylaminocarbonyl-C₁-C₂ alkylene or C₁-C₄haloalkylaminocarbonyl-C₁-C₂ alkylene, or group A

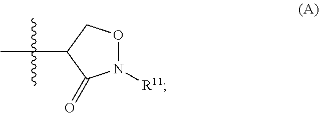

R³ is trifluoromethyl;

R⁴ is 3,5-dichloro-phenyl;

R⁵ is methyl;

each R⁸ is independently bromo, chloro, fluoro, cyano or methyl;

R¹¹ is methyl, ethyl or trifluoroethyl.

In a further group of compounds of formula (IA) P is P1, Q is Q^{A}1 or Q^{A}2. The preferred substituent definitions given above also apply to the substituents in the preferred groups of compounds of formula (IA) above in those cases where a preferred substituent definition is narrower.

In one embodiment the present invention provides compounds of formula (IAa)

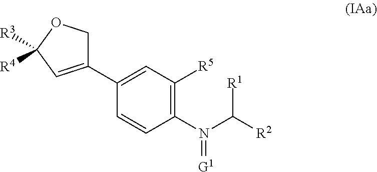

wherein G¹, R¹, R², R³, R⁴ and R⁵ are as defined for compounds of formula (IA); or a salt or N-oxide thereof. The preferences for G¹, R¹, R², R³, R⁴ and R⁵ are the same as the preferences set out for the corresponding substituents of compounds of the formula (IA).

In a further embodiment the present invention provides a mixture comprising a compound of formula (IAa) as defined above and a compound of formula (IBa)

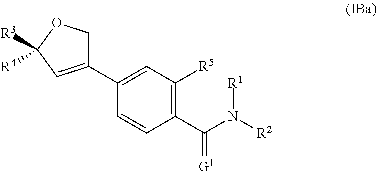

wherein G¹, R¹, R², R³, R⁴ and R⁵ and preferences thereof are as defined for compounds of formula (IA); and wherein the mixture is enriched for compounds of formula (IAa).

In a further embodiment the present invention provides compounds of formula (IAb)

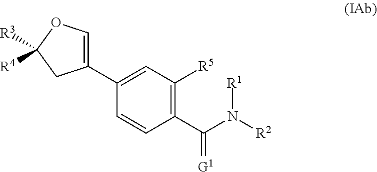

wherein G¹, R¹, R², R³, R⁴ and R⁵ are as defined for compounds of formula (IA); or a salt or N-oxide thereof. The preferences for G¹, R¹, R², R³, R⁴ and R⁵ are the same as the preferences set out for the corresponding substituents of compounds of the formula (IA).

In a further embodiment the present invention provides a mixture comprising a compound of formula (IAb) as defined above and a compound of formula (IBb)

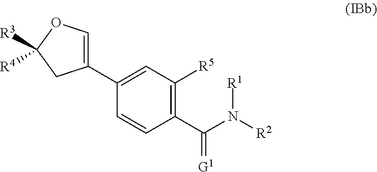

wherein G¹, R¹, R², R³, R⁴ and R⁵ and preferences thereof are as defined for compounds of formula (IA); and wherein the mixture is enriched for compounds of formula (IAb).

In a further embodiment the present invention provides compounds of formula (IAc)

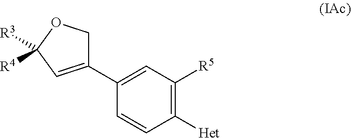

wherein R³, R⁴ and R⁵ are as defined for compounds of formula (IA) and Het is selected from H1 to H9 as defined for compounds of formula (IA); or a salt or N-oxide thereof. The preferences for R³, R⁴, R⁵, and Het are the same as the preferences set out for the corresponding substituents of compounds of the formula (IA).

In a further embodiment the present invention provides a mixture comprising a compound of formula (IAc) as defined above and a compound of formula (IBc)

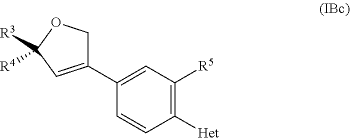

wherein R³, R⁴ and R⁵ are as defined for compounds of formula (IA) and Het is selected from H1 to H9 as defined for compounds of formula (IA) and wherein the mixture is enriched for compounds of formula (IAc). The preferences for R³, R⁴, R⁵, and Het are the same as the preferences set out for the corresponding substituents of compounds of the formula (IA).

In a further embodiment the present invention provides compounds of formula (IAd)

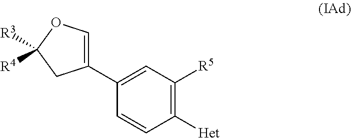

wherein R³, R⁴ and R⁵ are as defined for compounds of formula (IA) and Het is selected from H1 to H9 as defined for compounds of formula I; or a salt or N-oxide thereof. The preferences for R³, R⁴, R⁵, and Het are the same as the preferences set out for the corresponding substituents of compounds of the formula (IA).

In a further embodiment the present invention provides a mixture comprising a compound of formula (IAd) as defined above and a compound of formula (IBd)

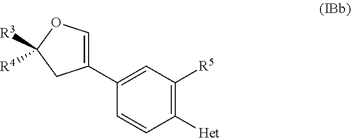

wherein R³, R⁴ and R⁵ and preferences thereof are as defined for compounds of formula (IA) and Het is selected from H1 to H9 as defined for compounds of formula (IA) and wherein the mixture is enriched for compounds of formula (IAd).

Certain intermediates are novel and as such form further aspects of the invention. One group of novel intermediates are compounds of formula (IIA)

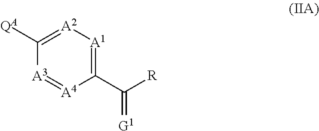

wherein Q^{A}, A¹, A², A³, and A⁴ are as defined for a compound of formula (IA), G¹ is oxygen, and R is hydroxy, C₁-C₁₅alkoxy or halogen, such as bromo, chloro or fluoro; or a salt or N-oxide thereof. The preferences for Q^{A}, A¹, A², A³, and A⁴ are the same as the preferences set out for the corresponding substituents of a compound of formula (IA). Preferably R is hydroxy, C₁-C₆alkoxy or chloro.

The invention also provides a mixture comprising a compound of formula (IIA) as defined above and a compound of formula (IIB)

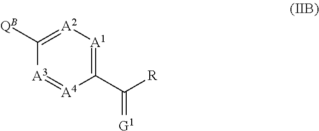

wherein A¹, A², A³, and A⁴ and preferences thereof are as defined for a compound of formula (IA), Q^{B} is as defined for a compound of formula (IB), G¹ is oxygen, and R is hydroxy, C₁-C₁₅alkoxy (e.g. C₁-C₆alkoxy) or halogen, such as bromo, chloro or fluoro, preferably hydroxy, C₁-C₆alkoxy or chloro, and wherein the mixture is enriched for compounds of formula (IIA).

One group of compounds of formula (IIA) are compounds of formula (IIAa)

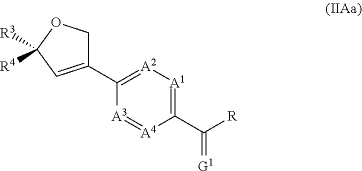

wherein A¹, A², A³, A⁴, R³ and R⁴ are as defined for a compound of formula (IA), G¹ is oxygen, and R is hydroxy, C₁-C₆alkoxy or halogen, such as bromo, chloro or fluoro; or a salt or N-oxide thereof. The preferences for A¹, A², A³, A⁴, R³ and R⁴ are the same as the preferences set out for the corresponding substituents of a compound of formula (IA). Preferably R is hydroxy, C₁-C₆alkoxy or chloro.

The invention also provides a mixture comprising a compound of formula (IIAa) as defined above and a compound of formula (IIBa)

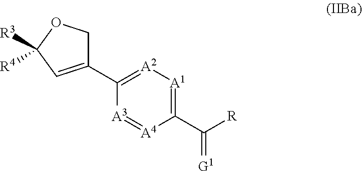

wherein A¹, A², A³, A⁴, R³ and R⁴ and preferences thereof are as defined for a compound of formula (IA), G¹ is oxygen, and R is hydroxy, C₁-C₆alkoxy or halogen, such as bromo, chloro or fluoro, and wherein the mixture is enriched for compounds of formula (IIAa).

A further group of compounds of formula IIA are compounds of formula (IIAb)

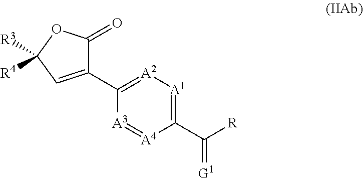

wherein A¹, A², A³, A⁴, R³ and R⁴ are as defined for a compound of formula (IA), G¹ is oxygen, and R is hydroxy, C₁-C₆alkoxy or halogen, such as bromo, chloro or fluoro; or a salt or N-oxide thereof. The preferences for A¹, A², A³, A⁴, R³ and R⁴ are the same as the preferences set out for the corresponding substituents of a compound of formula (IA). Preferably R is hydroxy, C₁-C₆alkoxy or chloro.

The invention also provides a mixture comprising a compound of formula (IIAb) as defined above and a compound of formula (IIBb)

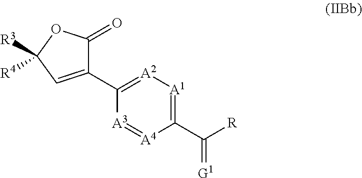

wherein A¹, A², A³, A⁴, R³ and R⁴ and preferences thereof are as defined for a compound of formula (IA), G¹ is oxygen, and R is hydroxy, C₁-C₆alkoxy or halogen, such as bromo, chloro or fluoro, and wherein the mixture is enriched for compounds of formula (IIAb).

A further group of compounds of formula (IIA) are compounds of formula (IIAc)

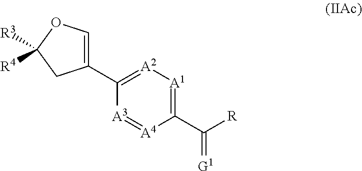

wherein A¹, A², A³, A⁴, R³ and R⁴ are as defined for a compound of formula (IA), G¹ is oxygen, and R is hydroxy, C₁-C₆alkoxy or halogen, such as bromo, chloro or fluoro; or a salt or N-oxide thereof. The preferences for A¹, A², A³, A⁴, R³ and R⁴ are the same as the preferences set out for the corresponding substituents of a compound of formula (IA). Preferably R is hydroxy, C₁-C₆alkoxy or chloro.

The invention also provides a mixture comprising a compound of formula (IIAc) as defined above and a compound of formula (IIBc)

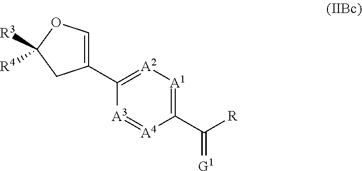

wherein A¹, A², A³, A⁴, R³ and R⁴ and preferences thereof are as defined for a compound of formula (IA), G¹ is oxygen, and R is hydroxy, C₁-C₆alkoxy or halogen, such as bromo, chloro or fluoro, and wherein the mixture is enriched for compounds of formula (IIAc). Preferably R is hydroxy, C₁-C₆alkoxy or chloro.

A further group of novel intermediates are compounds of formula (IIIA)

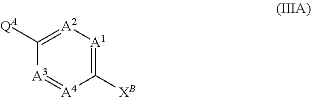

wherein Q^{A}, A¹, A², A³, and A⁴ are as defined for a compound of formula (IA), and X^{B} is a leaving group such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. X^{B} is —N₂⁺Cl⁻, —N₂⁺BF₄⁻, —N₂⁺Br⁻, —N₂⁺PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride; or a salt or N-oxide thereof. The preferences for Q^{A}, A¹, A², A³, and A⁴ are the same as the preferences set out for the corresponding substituents of a compound of formula (IA).

The invention also provides a mixture comprising a compound of formula (IIIA) as defined above and a compound of formula (IIIB)

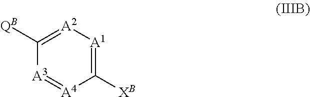

wherein A¹, A², A³, and A⁴ and preferences thereof are as defined for a compound of formula (IA), Q^{B} is as defined for compounds of formula (IB) and X^{B} is a leaving group such as halogen, C₁-C₈alkoxy, C₁-C₈ alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. X^{B} is —N₂⁺Cl⁻, —N₂⁺BF₄⁻, —N₂⁺Br⁻, —N₂⁺PF₆⁻), phosphonate esters (e.g. −OP(O)(OR)₂, wherein R is methyl or ethyl), preferably bromo, iodo, chloro, trifluoromethylsulfoxy, p-toluenesulfoxy, diazonium chloride, and wherein the mixture is enriched for compounds of formula (IIIA).

One group of compounds of formula (IIIA) are compounds of formula (IIIAa)

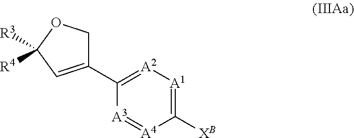

wherein A¹, A², A³, A⁴, R³ and R⁴ are as defined for a compound of formula (IA), and X^{B} is as defined for compounds of formula (IIIA). The preferences for A¹, A², A³, A⁴, R³ and R⁴ are the same as the preferences set out for the corresponding substituents of a compound of formula (IA).

The invention also provides a mixture comprising a compound of formula (IIIAa) as defined above and a compound of formula (IIIBa)

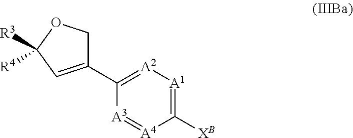

wherein A¹, A², A³, A⁴, R³ and R⁴ and preferences thereof are as defined for a compound of formula (IA), and X^{B} is as defined for compounds of formula (IIIA), and wherein the mixture is enriched for compounds of formula (IIIAa).

A further group of compounds of formula (IIIA) are compounds of formula (IIIAb)

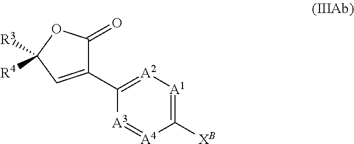

wherein A¹, A², A³, A⁴, R³ and R⁴ are as defined for a compound of formula (IA), and X^{B} is as defined for compounds of formula (IIIA). The preferences for A¹, A², A³, A⁴, R³ and R⁴ are the same as the preferences set out for the corresponding substituents of a compound of formula (IA).

The invention also provides a mixture comprising a compound of formula (IIIAb) as defined above and a compound of formula (IIIBb)

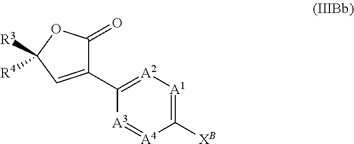

wherein A¹, A², A³, A⁴, R³ and R⁴ and preferences thereof are as defined for a compound of formula (IA), and X^{B} is as defined for compounds of formula (IIIA), and wherein the mixture is enriched for compounds of formula (IIIAb).

A further group of compounds of formula (IIIA) are compounds of formula (IIIAc)

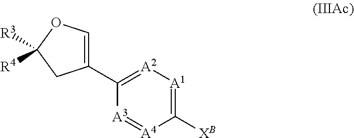

wherein A¹, A², A³, A⁴, R³ and R⁴ are as defined for a compound of formula (IA) and X^{B} is as defined for compounds of formula (IIIA). The preferences for A¹, A², A³, A⁴, R³ and R⁴ are the same as the preferences set out for the corresponding substituents of a compound of formula (IA).

The invention also provides a mixture comprising a compound of formula (IIIAc) as defined above and a compound of formula (IIIBc)

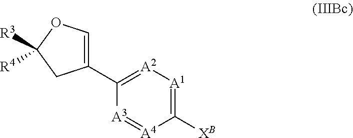

wherein A¹, A², A³, A⁴, R³ and R⁴ and preferences thereof are as defined for a compound of formula (IA) and X^{B} is as defined for compounds of formula (IIIA), and wherein the mixture is enriched for compounds of formula (IIIAb).

A further group of novel intermediates are compounds of formula (XXVA)

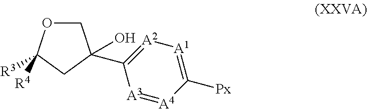

wherein A¹, A², A³, A⁴, R³ and R⁴ are as defined for compounds of formula (IA), Px is P as defined above (for the compound of formula (IA)), a leaving group X^{B}, such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. X^{B} is —N₂⁺Cl⁻, —N₂⁺BF₄⁻, —N₂⁺Br⁻, —N₂⁺PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride, or C(O)R wherein R is halogen, OH or C₁-C₁₅alkoxy, or a salt or N-oxide thereof. The preferences for A¹, A², A³, A⁴, R³, R⁴ and preferences at position Px when Px is P are the same as the preferences set out for the corresponding substituents of a compound of formula (IA).

The invention also provides a mixture comprising a compound of formula (XXVA) as defined above and a compound of formula (XXVB)

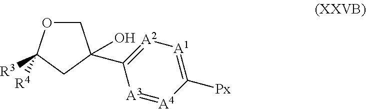

wherein A¹, A², A³, A⁴, R³ and R⁴ and preferences thereof are as defined for compounds of formula (IA), Px is P as defined above, a leaving group X^{B}, such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. X^{B} is —N₂⁺Cl⁻, —N₂⁺BF₄⁻, —N₂⁺Br⁻, —N₂⁺PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride, or C(O)R wherein R is halogen, OH or C₁-C₁₅alkoxy, and wherein the mixture is enriched for compounds of formula (XXVA).

One group of compounds of formula (XXVA) are compounds of (XXVAa)

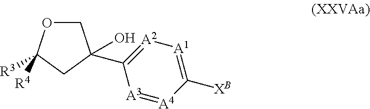

wherein A¹, A², A³, A⁴, R³ and R⁴ are as defined for compounds of formula (IA), and X^{B} is as defined for the compound of formula (XXVA). The preferences for A¹, A², A³, A⁴, R³, and R⁴ are the same as the preferences set out for the corresponding substituents of a compound of formula (IA).

The invention also provides a mixture comprising a compound of formula (XXVAa) as defined above and a compound of formula (XXVBa)

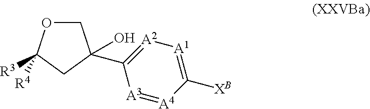

wherein A¹, A², A³, A⁴, R³ and R⁴ and preferences thereof are as defined for compounds of formula (IA), and X^{B} is as defined for the compound of formula (XXVAa), and wherein the mixture is enriched for compounds of formula (XXVAa).

Another group of compounds of formula (XXVA) are compounds of formula (XXVAb)

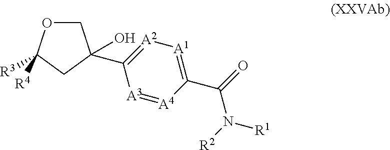

wherein A¹, A², A³, A⁴, R¹, R², R³ and R⁴ are as defined for compounds of formula (IA). The preferences for A¹, A², A³, A⁴, R¹, R², R³, and R⁴ are the same as the preferences set out for the corresponding substituents of a compound of formula (IA).

The invention also provides a mixture comprising a compound of formula (XXVAb) as defined above and a compound of formula (XXVBb)

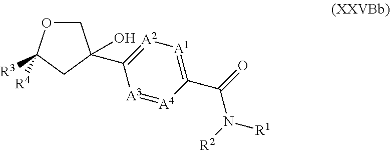

wherein A¹, A², A³, A⁴, R¹, R², R³ and R⁴ and preferences thereof are as defined for compounds of formula (IA), and X^{B} is as defined for the compound of formula (XXVa), and wherein the mixture is enriched for compounds of formula (XXVAb).

Another group of compounds of formula (XXVA) are compounds of formula (XXVAc)

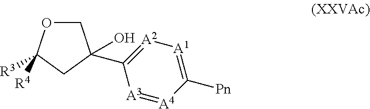

wherein A¹, A², A³, A⁴, R³ and R⁴ are as defined for the compounds of formula (IA), and Pn is a heterocyclyl or heterocyclyl substituted by one to five Z, as defined for compounds of formula I. The preferences for A¹, A², A³, A⁴, R³, R⁴ and heterocyclyl are the same as the preferences set out for the corresponding substituents of a compound of formula (IA).

The invention also provides a mixture comprising a compound of formula (XXVAc) as defined above and a compound of formula (XXVBc)

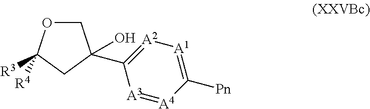

wherein A¹, A², A³, A⁴, R³ and R⁴ and preferences thereof are as defined for the compounds of formula (IA), and Pn is a heterocyclyl or heterocyclyl substituted by one to five Z, as defined for compounds of formula (IA), and wherein the mixture is enriched for compounds of formula (XXVAc).

Another group of novel intermediates are compounds of formula (XXIIIA)

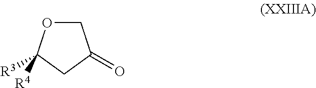

wherein R³ and R⁴ are as defined for the compounds of formula (IA), or a salt or N-oxide thereof. The preferences for R³ and R⁴ are the same as the preferences set out for the corresponding substituents of a compound of formula (IA).

A preferred group of compounds of formula (XXIIIA) are those wherein R³ is CF₃ and R⁴ is phenyl substituted by 1 to 5 R⁹.

The invention also provides a mixture comprising a compound of formula (XXIIIA) as defined above and a compound of formula (XXIIIB)

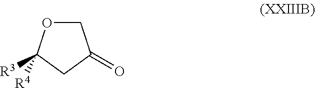

wherein R³ and R⁴ and preferences thereof are as defined for the compounds of formula (IA), e.g. R³ is CF₃ and R⁴ is phenyl substituted by 1 to 5 R⁹, and wherein the mixture is enriched for compounds of formula (XXIIIA).

Another group of novel intermediates are compounds of formula (XXVIA)

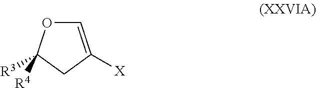

wherein R³ and R⁴ and preferences thereof are as defined for compounds of formula I, and X is a tin derivative, e.g. SnR¹³₃ wherein each R¹³ is independently C₁-C₆ alkyl, a boron derivative, e.g. BF₃, B(OH)₂ or B(OR)₂, wherein each R is independently C₁-C₆-alkyl, C₁-C₆-cycloalkyl or optionally substituted C₁-C₆-cycloalkyl, e.g. optionally substituted by halogen, C₁-C₄ alkyl or C₁-C₄haloalkyl, a leaving group, such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. is —N₂⁺Cl⁻, —N₂⁺BF₄⁻, —N₂⁺Br⁻, —N₂⁺PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride, or C(O)R wherein R is halogen, OH or C₁-C₁₅alkoxy or a salt or N-oxide thereof. A preferred group of compounds of formula (XXVIA) are those wherein R³ is CF₃ and R⁴ is phenyl substituted by 1 to 5 R⁹.

The invention also provides a mixture comprising a compound of formula (XXVIA) as defined above and a compound of formula (XXVIB)

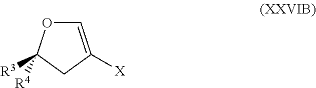

wherein R³ and R⁴ and preferences thereof are as defined for compounds of formula (IA), e.g. R³ is CF₃ and R⁴ is phenyl substituted by 1 to 5 R⁹, and X is a tin derivative, e.g. SnR¹³₃ wherein each R¹³ is independently C₁-C₆ alkyl, a boron derivative, e.g. BF₃, B(OH)₂ or B(OR)₂, wherein each R is independently C₁-C₆-alkyl, C₁-C₆-cycloalkyl or optionally substituted C₁-C₆-cycloalkyl, e.g. optionally substituted by halogen, C₁-C₄ alkyl or C₁-C₄haloalkyl, a leaving group, such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. —N₂⁺Cl⁻, —N₂⁺BF₄⁻, —N₂+Br⁻, —N₂⁺PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride, or C(O)R wherein R is halogen, OH or C₁-C₁₅alkoxy and wherein the mixture is enriched for compounds of formula (XXVIA).

Another group of novel intermediates are compounds of formula (XXVIA′)

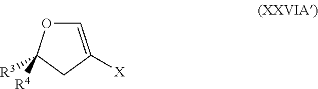

wherein R³ and R⁴ and preferences thereof are as defined for compounds of formula (IA), e.g. R³ is CF₃ and R⁴ is phenyl substituted by 1 to 5 R⁹, and X is Cl, Br or I, or a salt or N-oxide thereof (Compounds of formula XXVIA′ are a preferred group of compounds of formula XXVIA.)

The invention also provides a mixture comprising a compound of formula (XXVIA′) as defined above and a compound of formula (XXVIB′)

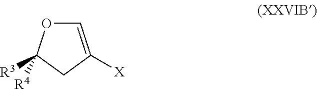

wherein R³ and R⁴ and preferences thereof are as defined for compounds of formula (IA), e.g. R³ is CF₃ and R⁴ is phenyl substituted by 1 to 5 R⁹, and X is Cl, Br or I, and wherein the mixture is enriched for compounds of formula (XXVIA′).

Another group of novel intermediates are compounds of formula (XXVIIA)

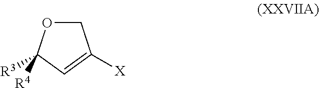

wherein R³ and R⁴ and preferences thereof are as defined for compounds of formula (IA), and X is as defined for compounds of formula (XXVIA), or a salt or N-oxide thereof A preferred group of compounds of formula (XXVIIA) are those wherein R³ is CF₃ and R⁴ is phenyl substituted by 1 to 5 R⁹.

The invention also provides a mixture comprising a compound of formula (XXVIIA) as defined above and a compound of formula (XXVIIB)

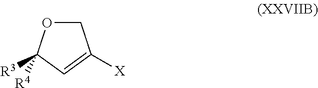

wherein R³ and R⁴ and preferences thereof are as defined for compounds of formula I, e.g. R³ is CF₃ and R⁴ is phenyl substituted by 1 to 5 R⁹, and X is as defined for compounds of formula (XXVIA), and wherein the mixture is enriched for compounds of formula (XXVIIA).

Another group of novel intermediates are compounds of formula (XXVIIA′)

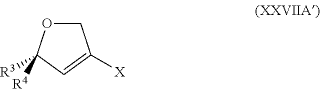

wherein R³ and R⁴ and preferences thereof are as defined for compounds of formula (IA), and X is Cl, Br or I, or a salt or N-oxide thereof. A preferred group of compounds of formula (XXVIIA′) are those wherein R³ is CF₃ and R⁴ is phenyl substituted by 1 to 5 R⁹. (Compounds of formula XXVIIA′ are a preferred group of compounds of formula XXVIA.)

The invention also provides a mixture comprising a compound of formula (XXVIIA′) as defined above and a compound of formula (XXVIIB′)

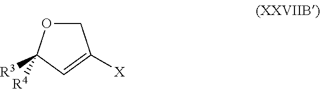

wherein R³ and R⁴ and preferences thereof are as defined for compounds of formula (IA), e.g. R^{3 is CF}₃ and R⁴ is phenyl substituted by 1 to 5 R⁹, and X is Cl, Br or I, and wherein the mixture is enriched for compounds of formula (XXVIIA′).

Another group of novel intermediates are compounds of formula (XXXVIA)

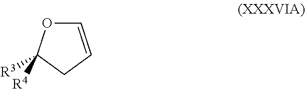

wherein R³ and R⁴ and preferences thereof are as defined for compounds of formula (IA), or a salt or N-oxide thereof. A preferred group of compounds of formula (XXXVIA) are those wherein R³ is CF₃ and R⁴ is phenyl substituted by 1 to 5 R⁹.

The invention also provides a mixture comprising a compound of formula (XXXVIA) as defined above and a compound of formula (XXXVIB)

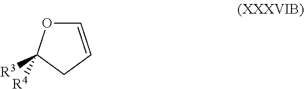

wherein R³ and R⁴ and preferences thereof are as defined for compounds of formula (IA), e.g. R³ is CF₃ and R⁴ is phenyl substituted by 1 to 5 R⁹, and wherein the mixture is enriched for compounds of formula (XXXVIA).

Another group of novel intermediates are compounds of formula (XXXVIIA)

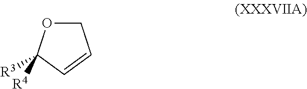

wherein R³ and R⁴ and preferences thereof are as defined for compounds of formula (IA), or a salt or N-oxide thereof. A preferred group of compounds of formula (XXXVIIA) are those wherein R³ is CF₃ and R⁴ is phenyl substituted by 1 to 5 R⁹.

The invention also provides a mixture comprising a compound of formula (XXXVIIA) as defined above and a compound of formula (XXXVIIB)

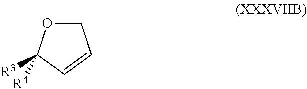

wherein R³ and R⁴ and preferences thereof are as defined for compounds of formula (IA), e.g. R³ is CF₃ and R⁴ is phenyl substituted by 1 to 5 R⁹, and wherein the mixture is enriched for compounds of formula (XXXVIIA).

Another group of novel intermediates are compounds of formula (XXXVIIIA)

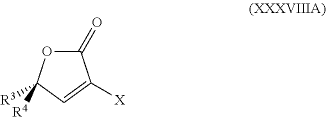

wherein R³ and R⁴ and preferences thereof are as defined for compounds of formula (IA), and X is as defined for compounds of formula (XXVIA), or a salt or N-oxide thereof A preferred group of compounds of formula (XXXVIIIA) are those wherein R³ is CF₃ and R⁴ is phenyl substituted by 1 to 5 R⁹.

The invention also provides a mixture comprising a compound of formula (XXXVIIIA) as defined above and a compound of formula (XXXVIIIB)

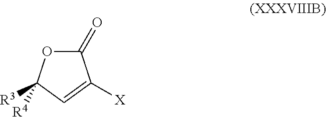

wherein R³ and R⁴ and preferences thereof are as defined for compounds of formula (IA), e.g. R³ is CF₃ and R⁴ is phenyl substituted by 1 to 5 R⁹, and X is as defined for compounds of formula (XXVIA), wherein the mixture is enriched for compounds of formula (XXXVIIIA).

Another group of novel intermediates are compounds of formula (LA)

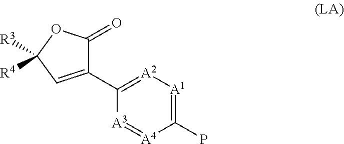

wherein R³, R⁴, A¹, A², A³, A⁴ and P are as defined for compounds of formula (IA), or a salt or N-oxide thereof. The preferences for R³, R⁴, A¹, A², A³, A⁴ and P are the same as the preferences set out for the corresponding substituents of a compound of formula (IA).

The invention also provides a mixture comprising a compound of formula (LA) as defined above and a compound of formula (LB)

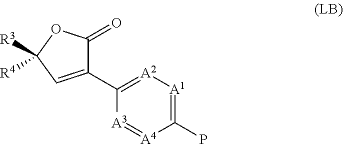

wherein R³, R⁴, A¹, A², A³, A⁴ and P and preferences thereof are as defined for compounds of formula (IA), and wherein the mixture is enriched for compounds of formula (LA).

Another group of novel intermediates are compounds of formula (LXA)

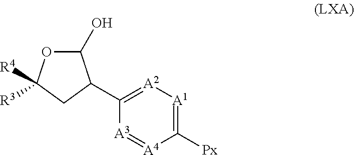

wherein R³, R⁴, A¹, A², A³, A⁴ are as defined for the compound of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of the compound of formula (IA).

The invention also provides a mixture comprising a compound of formula (LXA) as defined above and a compound of formula (LXB)

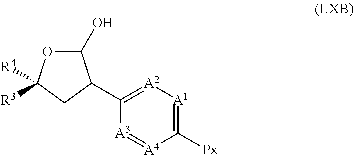

wherein R³, R⁴, A¹, A², A³, A⁴ and preferences thereof are as defined for the compound of formula (IA) and Px is as defined for the compound of formula (XXVA), and wherein the mixture is enriched for compounds of formula (LXA).

The compound of formula (LXA) includes compounds of formula (LXA′) which can exist in equilibrium with compounds of formula (LXA)

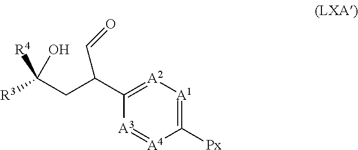

wherein R³, R⁴, A¹, A², A³, A⁴ are as defined for the compound of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of the compound of formula (IA).

One group of compounds of formula (LXA) are compounds of (LXAa)

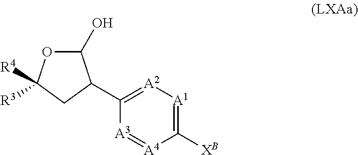

wherein R³, R⁴, A¹, A², A³, A⁴ are as defined for the compound of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of the compound of formula (IA).

The invention also provides a mixture comprising a compound of formula (LXAa) as defined above and a compound of formula (LXBa)

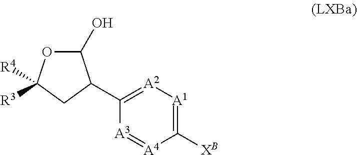

wherein R³, R⁴, A¹, A², A³, A⁴ and preferences thereof are as defined for the compound of formula (IA) and X^{B} is as defined for the compound of formula (XXVA), and wherein the mixture is enriched for compounds of formula (LXAa).

Another group of compounds of formula (LXA) are compounds of (LXAb)

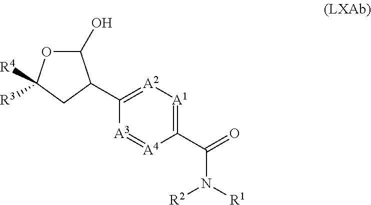

wherein R¹, R², R³, R⁴, A¹, A², A³ and A⁴ are as defined for the compound of formula (IA), or a salt or N-oxide thereof. The preferences for R¹, R², R³, R⁴, A¹, A², A³, A⁴ are the same as the preferences set our for the corresponding substituents of the compound of formula (IA).

The invention also provides a mixture comprising a compound of formula (LXAb) as defined above and a compound of formula (LXBb)

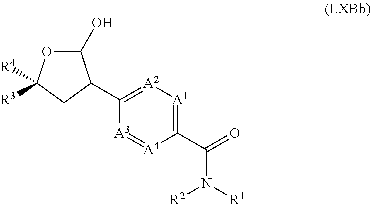

wherein R¹, R², R³, R⁴, A¹, A², A³, A⁴ and preferences thereof are as defined for the compound of formula (IA), and wherein the mixture is enriched for compounds of formula (LXAb).

Another group of compounds of formula (LXA) are compounds of (LXAc)

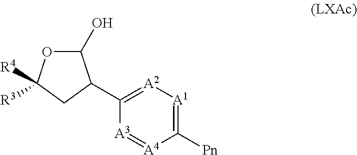

wherein A¹, A², A³, A⁴, R³ and R⁴ are as defined for the compounds of formula (IA), and Pn is a heterocyclyl or heteocyclyl substituted by one to five Z, as defined for compounds of formula I. The preferences for A¹, A², A³, A⁴, R³, R⁴ and heterocyclyl are the same as the preferences set out for the corresponding substituents of a compound of formula (IA).

The invention also provides a mixture comprising a compound of formula (LXAc) as defined above and a compound of formula (LXBc)

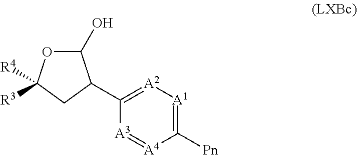

wherein A¹, A², A³, A⁴, R³ and R⁴ and preferences thereof are as defined for the compounds of formula (IA), and Pn is a heterocyclyl or heteocyclyl substituted by one to five Z, as defined for compounds of formula (IA), and wherein the mixture is enriched for compounds of formula (LXAc).

Another group of novel intermediates are compounds of formula (LIXA)

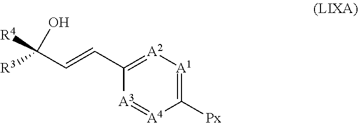

wherein R³, R⁴, A¹, A², A³, A⁴

A are as defined for the compound of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of the compound of formula (IA).

The invention also provides a mixture comprising a compound of formula (LIXA) as defined above and a compound of formula (LIXB)

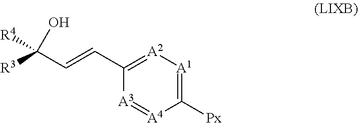

wherein R³, R⁴, A¹, A², A³, A⁴ and preferences thereof are as defined for the compound of formula (IA) and Px is as defined for the compound of formula (XXVA), and wherein the mixture is enriched for compounds of formula (LIXA).

One group of compounds of formula (LIXA) are compounds of (LIXAa)

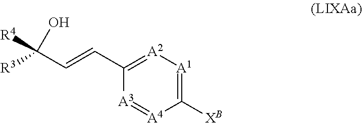

wherein R³, R⁴, A¹, A², A³, A⁴ are as defined for the compound of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of the compound of formula (IA).

The invention also provides a mixture comprising a compound of formula (LIXAa) as defined above and a compound of formula (LIXBa)

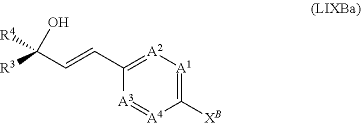

wherein R³, R⁴, A¹, A², A³, A⁴ and preferences thereof are as defined for the compound of formula (IA) and X^{B} is as defined for the compound of formula (XXVA), and wherein the mixture is enriched for compounds of formula (LIXAa).

Another group of compounds of formula (LIXA) are compounds of (LIXAb)

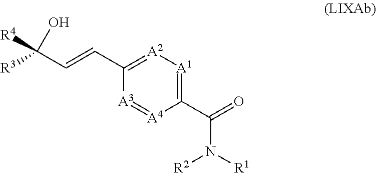

wherein R¹, R², R³, R⁴, A¹, A², A³, A⁴ are as defined for the compound of formula (IA), or a salt or N-oxide thereof. The preferences for R³, R⁴, A¹, A², A³, A⁴ are the same as the preferences set out for the corresponding substituents of the compound of formula (IA).

The invention also provides a mixture comprising a compound of formula (LIXAb) as defined above and a compound of formula (LIXBb)

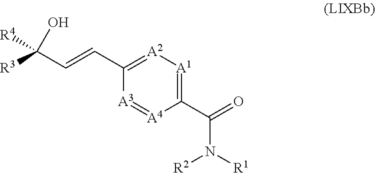

wherein R¹, R², R³, R⁴, A¹, A², A³, A⁴ and preferences thereof are as defined for the compound of formula (IA), and wherein the mixture is enriched for compounds of formula (LIXAb).

Another group of compounds of formula (LIXA) are compounds of (LIXAc)

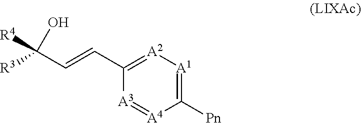

wherein A¹, A², A³, A⁴, R³ and R⁴ are as defined for the compounds of formula (IA), and Pn is a heterocyclyl or heteocyclyl substituted by one to five Z, as defined for compounds of formula I. The preferences for A¹, A², A³, A⁴, R³, R⁴ and heterocyclyl are the same as the preferences set out for the corresponding substituents of a compound of formula (IA).

The invention also provides a mixture comprising a compound of formula (LIXAc) as defined above and a compound of formula (LIXBc)

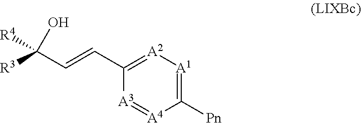

wherein A¹, A², A³, A⁴, R³ and R⁴ and preferences thereof are as defined for the compounds of formula (IA), and Pn is a heterocyclyl or heteocyclyl substituted by one to five Z, as defined for compounds of formula (IA), and wherein the mixture is enriched for compounds of formula (LIXAc).

Another group of novel intermediates are compounds of formula (LIIA)

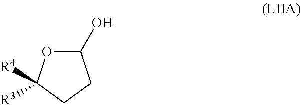

wherein R³ and R⁴ are as defined for compounds of formula (IA), or a salt or N-oxide thereof. The preferences for R³ and R⁴ are the same as the preferences set out for the corresponding substituents of a compound of formula (IA).

The invention also provides a mixture comprising a compound of formula (LIIA) as defined above and a compound of formula (LIIB)

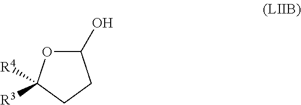

wherein R³ and R⁴ and preferences thereof are as defined for compounds of formula (IA), and wherein the mixture is enriched for compounds of formula (LIIA).

The compound of formula (LIIA) includes compounds of formula (LIIA′) which can exist in equilibrium with compounds of formula (LIIA)

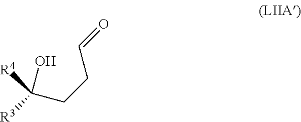

wherein R³ and R⁴ are as defined for compounds of formula (IA), or a salt or N-oxide thereof. The preferences for R³ and R⁴ are the same as the preferences set out for the corresponding substituents of a compound of formula (IA).

Another group of novel intermediates are compounds of formula (LIIIA)

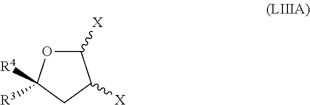

wherein R³ and R⁴ are as defined for compounds of formula (IA), and each X independently represents Cl, Br or I, or a salt or N-oxide thereof. The preferences for R³ and R⁴ are the same as the preferences set out for the corresponding substituents of a compound of formula (IA).

The invention also provides a mixture comprising a compound of formula (LIIIA) as defined above and a compound of formula (LIIIB)

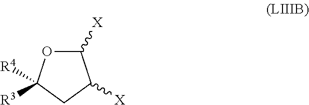

wherein R³ and R⁴ and preferences thereof are as defined for compounds of formula (IA), and each X independently represents Cl, Br or I, and wherein the mixture is enriched for compounds of formula (LIIIA).

Another group of novel intermediates are compounds of formula (LVA)

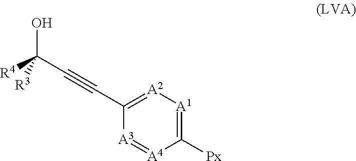

wherein R³, R⁴, A¹, A², A³ and A⁴ are as defined for compounds of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of a compound of formula (IA).

The invention also provides a mixture comprising a compound of formula (LVA) as defined above and a compound of formula (LVB)

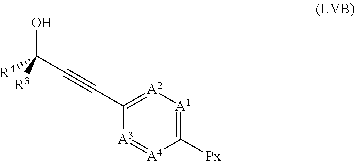

wherein R³, R⁴, A¹, A², A³ and A⁴ and preferences thereof are as defined for compounds of formula (IA), and Px is as defined for the compound of formula (XXVA), and wherein the mixture is enriched for compounds of formula (LVA).

One group of compounds of formula (LVA) are compounds of (LVAa)

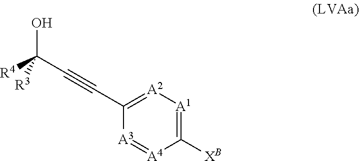

wherein R³, R⁴, A¹, A², A³, A⁴ are as defined for the compound of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of the compound of formula (IA).

The invention also provides a mixture comprising a compound of formula (LVAa) as defined above and a compound of formula (LVBa)

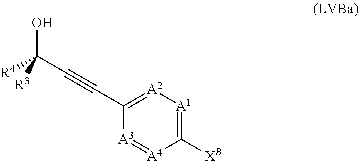

wherein R³, R⁴, A¹, A², A³, A⁴ and preferences thereof are as defined for the compound of formula (IA) and X^{B} is as defined for the compound of formula (XXVA), and wherein the mixture is enriched for compounds of formula (LVAa).

Another group of compounds of formula (LVA) are compounds of (LVAb)

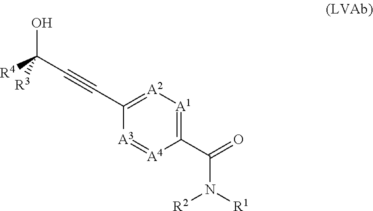

wherein R¹, R², R³, R⁴, A¹, A², A³, A⁴ are as defined for the compound of formula (IA), or a salt or N-oxide thereof. The preferences for R³, R⁴, A¹, A², A³, A⁴ are the same as the preferences set out for the corresponding substituents of the compound of formula (IA).

The invention also provides a mixture comprising a compound of formula (LVAb) as defined above and a compound of formula (LVBb)

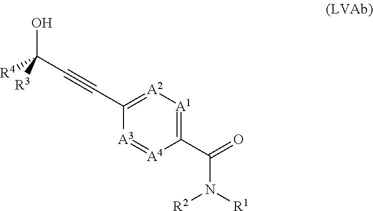

wherein R¹, R², R³, R⁴, A¹, A², A³, A⁴ and preferences thereof are as defined for the compound of formula (IA), and wherein the mixture is enriched for compounds of formula (LVAb).

Another group of compounds of formula (LVA) are compounds of (LVAc)

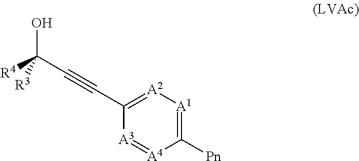

wherein A¹, A², A³, A⁴, R³ and R⁴ are as defined for the compounds of formula (IA), and Pn is a heterocyclyl or heteocyclyl substituted by one to five Z, as defined for compounds of formula I. The preferences for A¹, A², A³, A⁴, R³, R⁴ and heterocyclyl are the same as the preferences set out for the corresponding substituents of a compound of formula (IA).

The invention also provides a mixture comprising a compound of formula (LVAc) as defined above and a compound of formula (LVBc)

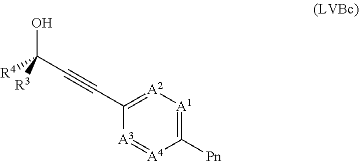

wherein A¹, A², A³, A⁴, R³ and R⁴ and preferences thereof are as defined for the compounds of formula (IA), and Pn is a heterocyclyl or heteocyclyl substituted by one to five Z, as defined for compounds of formula (IA), and wherein the mixture is enriched for compounds of formula (LVAc).

The invention also provides compounds of formula (LVIA)

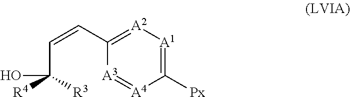

wherein R³, R⁴, A¹, A², A³ and A⁴ are as defined for compounds of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of a compound of formula (IA).

The invention also provides a mixture comprising a compound of formula (LIXAa) as defined above and a compound of formula (LIXBa)

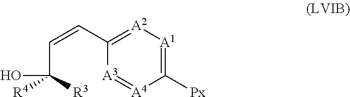

wherein R³, R⁴, A¹, A², A³, A⁴ and preferences thereof are as defined for the compound of formula (IA) and Px is as defined for the compound of formula (XXVA), and wherein the mixture is enriched for compounds of formula (LVIA).

One group of compounds of formula (LVIA) are compounds of (LVIAa)

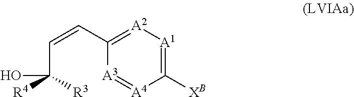

wherein R³, R⁴, A¹, A², A³, A⁴ are as defined for the compound of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of the compound of formula (IA).

The invention also provides a mixture comprising a compound of formula (LVIAa) as defined above and a compound of formula (LVIBa)

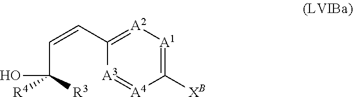

wherein R³, R⁴, A¹, A², A³, A⁴ and preferences thereof are as defined for the compound of formula (IA) and X^{B} is as defined for the compound of formula (XXVA), and wherein the mixture is enriched for the compound of formula (LVIAa)

Another group of compounds of formula (LVIA) are compounds of (LVIAb)

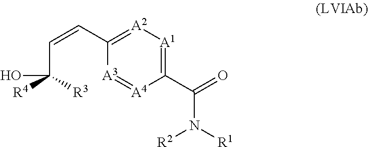

wherein R¹, R², R³, R⁴, A¹, A², A³, A⁴ are as defined for the compound of formula (IA), or a salt or N-oxide thereof. The preferences for R¹, R², R³, R⁴, A¹, A², A³, A⁴ are the same as the preferences set out for the corresponding substituents of the compound of formula (IA).

The invention also provides a mixture comprising a compound of formula (LVIAb) as defined above and a compound of formula (LVIBb)

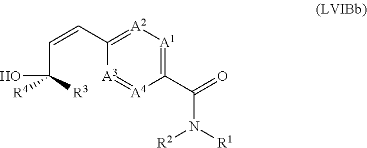

wherein R¹, R², R³, R⁴, A¹, A², A³, A⁴ and preferences thereof are as defined for the compound of formula (IA), and wherein the mixture is enriched for compounds of formula (LVIAb).

Another group of compounds of formula (LVIA) are compounds of (LVIAc)

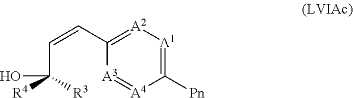

wherein A¹, A², A³, A⁴, R³ and R⁴ are as defined for the compounds of formula (IA), and Pn is a heterocyclyl or heteocyclyl substituted by one to five Z, as defined for compounds of formula (IA). The preferences for A¹, A², A³, A⁴, R³, R⁴ and heterocyclyl are the same as the preferences set out for the corresponding substituents of a compound of formula (IA).

The invention also provides a mixture comprising a compound of formula (LVIAc) as defined above and a compound of formula (LVIBc)

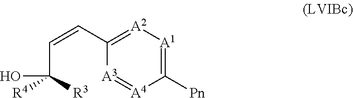

wherein A¹, A², A³, A⁴, R³ and R⁴ and preferences thereof are as defined for the compounds of formula (IA), and Pn is a heterocyclyl or heteocyclyl substituted by one to five Z, as defined for compounds of formula (IA), and wherein the mixture is enriched for compounds of formula (LVIAc).

In enriched mixtures of the invention, (also including the compounds described in the tables below), the molar proportion of the enriched compound in the mixture (e.g. the compound of formula (IA)) compared to the total amount of both compounds (e.g. the total amount of the compound of formula (IA) and the compound of formula ((IB)) is for example greater than 50%, e.g. at least 55, 60, 65, 70, 75, 80, 85, 90, 95, 96, 97, 98, or at least 99%. This also applies to the intermediate mixtures described above.

Racemic mixtures of compounds of formula (IA) are disclosed in PCT/EP2011/051284. This application is incorporated by reference.

The compounds in the Tables below illustrate the compounds of the invention. (Although the compounds in the tables below use different substituent identifiers for substituents as compared to the compounds described above, the identity of the compounds is clear.)

TABLE G

| Group number | R² |
|---|---|
| G.001 | ethyl- |
| G.002 | butyl- |
| G.003 | but-2-yl- |
| G.004 | 3-bromo-propyl- |
| G.005 | 2,2,2-trifluoro-ethyl- |
| G.006 | 3,3,3-trifluoro-propyl- |
| G.007 | 2-methoxy-ethyl- |
| G.008 | 1-methoxy-prop-2-yl- |
| G.009 | cyclobutyl- |
| G.010 | 2-methyl-cyclohex-1-yl- |
| G.011 | phenyl-methyl- |
| G.012 | 1-phenyl-eth-1-yl- |
| G.013 | 2-phenyl-eth-1-yl- |
| G.014 | (3-chloro-phenyl)-methyl- |
| G.015 | (2-fluoro-phenyl)-methyl- |
| G.016 | (4-methoxy-phenyl)-methyl- |
| G.017 | (2-trifluoromethyl-phenyl)-methyl- |
| G.018 | (2-trifluoromethoxy-phenyl)-methyl- |
| G.019 | (pyrid-2-yl)-methyl- |
| G.020 | (pyrid-3-yl)-methyl- |
| G.021 | (2-chloro-pyrid-5-yl)-methyl- |
| G.022 | (1-methyl-1H-imidazol-4-yl)-methyl- |
| G.023 | (furan-2-yl)-methyl- |
| G.024 | 2-(thiophen-2′-yl)-eth-1-yl- |
| G.025 | 2-(indol-3′-yl)-eth-1-yl- |
| G.026 | (1H-benzimidazol-2-yl)-methyl- |
| G.027 | (oxetan-2-yl)-methyl- |
| G.028 | (tetrahydrofuran-2-yl)-methyl- |
| G.029 | 2-([1′,3′]dioxolan-2′-yl)-eth-1-yl- |
| G.030 | 2-(morpholin-4′-yl)-eth-1-yl- |
| G.031 | 2-(benzo[1′,3′]dioxol-5′-yl)-eth-1-yl- |
| G.032 | (2,3-dihydro-benzo[1,4]dioxin-6-yl)-methyl- |
| G.033 | 2-chloro-phenyl- |
| G.034 | 3-fluoro-phenyl- |
| G.035 | 2-methyl-phenyl- |
| G.036 | 2-chloro-6-methyl-phenyl- |
| G.037 | 2-trifluoromethyl-phenyl- |
| G.038 | 2,4-dimethoxy-phenyl- |
| G.039 | 3-methyl-pyrid-2-yl- |
| G.040 | 1,3-dimethyl-1H-pyrazol-5-yl- |
| G.041 | 4-methyl-thiazol-2-yl- |
| G.042 | 5-methyl-thiadiazol-2-yl- |
| G.043 | quinolin-2-yl- |
| G.044 | quinolin-5-yl- |
| G.045 | benzothiazol-6-yl- |
| G.046 | 4-methyl-benzothiazol-2-yl- |
| G.047 | thietan-3-yl- |
| G.048 | 1-oxo-thietan-3-yl- |
| G.049 | 1,1-dioxo-thietan-3-yl- |
| G.050 | 3-methyl-thietan-3-yl- |
| G.051 | N-(2,2,2-Trifluoro-ethyl)-acetamide-2-yl |
| G.052 | thietan-2-yl-methyl- |
| G.053 | 1-oxo-thietan-2-yl-methyl- |
| G.054 | 1,1-dioxo-thietan-2-yl-methyl- |
| G.055 | thietan-3-yl-methyl- |
| G.056 | 1-oxo-thietan-3-yl-methyl- |
| G.057 | 1,1-dioxo-thietan-3-yl-methyl- |
| G.058 | thietan-3-yl-ethyl- |
| G.059 | 1-oxo-thietan-3-yl-ethyl- |
| G.060 | 1,1-dioxo-thietan-3-yl-ethyl- |
| G.061 | 2-fluoro-cyclopropyl |
| G.062 | n-Butyl |
| G.063 | 2-Methoxy-1-methyl-ethyl |
| G.064 | 1-Oxo-thietan-3-yl |
| G.065 | 2-ethyl-isoxazolidin-3-one-4-yl |
| G.066 | Dihydro-thiophen-2-one-3-yl |
| G.067 | 6-Ethoxycarbonyl-cyclohex-3-enyl |
| G.068 | 2-Benzylsulfanyl-ethyl |
| G.069 | 4-Methanesulfonyl-benzyl |
| G.070 | N′,N′-Dimethylamino-ethyl |
| G.071 | sec-Butyl |
| G.072 | Butan-1-ol-2-yl |
| G.073 | 2,2-Difluoro-ethyl |
| G.074 | Ethynyl-cyclohexyl |
| G.075 | 2-Morpholin-4-yl-ethyl |
| G.076 | 3-Pyrrolidin-1-yl-propyl |
| G.077 | 3-Piperidin-1-yl-propyl |
| G.078 | [3-(4-Chloro-phenyl)-isoxazol-5-yl]-methyl |
| G.079 | Phenethyl |
| G.080 | 1,2,2,6,6-Pentamethyl-piperidin-4-yl |
| G.081 | 2-Phenoxy-ethyl |
| G.082 | 3-Chloro-benzyl |
| G.083 | 2-Acetylamino-ethyl |
| G.084 | 4-Pyrazol-1-yl-benzyl |
| G.085 | 2-Methylsulfanyl-ethyl |
| G.086 | 2-Piperidin-1-yl-benzyl |
| G.087 | 4-Phenoxy-benzyl |
| G.088 | (6-Chloro-pyridin-3-yl)-methyl |
| G.089 | 1-Benzyl-pyrrolidin-3-yl |
| G.090 | 2-(4-Benzyl-piperazin-1-yl)-ethyl |
| G.091 | Furan-2-yl-methyl |
| G.092 | 1H-Indazol-5-yl |
| G.093 | 4-Pyrrol-1-yl-phenyl |
| G.094 | 4-Piperidin-1-yl-phenyl |
| G.095 | 2-Methylsulfanyl-phenyl |
| G.096 | 4-Methyl-2-oxo-2H-chromen-7-yl |
| G.097 | 4-Dimethylsulfamoyl-phenyl |
| G.098 | 2,5-Dimethyl-2H-pyrazol-3-yl |
| G.099 | 5-Methylsulfanyl-1H-[1,2,4]triazol-3-yl |
| G.100 | 4-Hydroxy-6-methyl-pyrimidin-2-yl |
| G.101 | Quinolin-2-yl |
| G.102 | 5-Methyl-3-phenyl-isoxazol-4-yl |
| G.103 | 9H-Purin-6-yl |
| G.104 | 5-Acetyl-4-methyl-thiazol-2-yl |
| G.105 | 4-Methyl-benzothiazol-2-yl |
| G.106 | 5-Methyl-[1,3,4]thiadiazol-2-yl |
| G.107 | 4,6-Dimethyl-2H-pyrazolo[3,4-b]pyridin-3-yl |
| G.108 | 3-(2,2,2-Trifluoro-ethoxyimino)-cyclobutyl |
| G.109 | 2-Thietan-3-yl-ethyl |
| G.110 | 2-(1,1-Dioxo-thietan-3-yl)-ethyl |
| G.111 | 3-Oxo-2-(2,2,2-trifluoro-ethyl)-isoxazolidin-4-yl |
| G.112 | 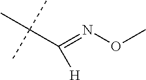 |
| G.113 | 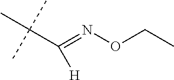 |
| G.114 | 2-(2,2,2-trifluoro-ethyl)-isoxazolidin-3-one-4-yl |
| G.115 | 2-(2,2-Difluoro-ethyl)-isoxazolidin-3-one-4-yl |
| G.116 | 2-(2-Fluoro-ethyl)-isoxazolidin-3-one-4-yl |

TABLE P

| | X1, X2, X3 | X4 | R5 | Y |
|---|---|---|---|---|
| P.0001 | X¹ is chloro, X² is CH, X³ is | chlorodifluoromethyl | bromo | Y¹ is CH, Y² is |
| | chloro | | | CH, Y³ is CH |
| P.0002 | X¹ is chloro, X² is CH, X³ is | chlorodifluoromethyl | chloro | Y¹ is CH, Y² is |
| | chloro | | | CH, Y³ is CH |
| P.0003 | X¹ is chloro, X² is CH, X³ is | chlorodifluoromethyl | cyano | Y¹ is CH, Y² is |
| | chloro | | | CH, Y³ is CH |
| P.0004 | X¹ is chloro, X² is CH, X³ is | chlorodifluoromethyl | cyclopropyl | Y¹ is CH, Y² is |
| | chloro | | | CH, Y³ is CH |
| P.0005 | X¹ is chloro, X² is CH, X³ is | chlorodifluoromethyl | ethyl | Y¹ is CH, Y² is |
| | chloro | | | CH, Y³ is CH |
| P.0006 | X¹ is chloro, X² is CH, X³ is | chlorodifluoromethyl | fluoro | Y¹ is CH, Y² is |
| | chloro | | | CH, Y³ is CH |
| P.0007 | X¹ is chloro, X² is CH, X³ is | chlorodifluoromethyl | hydrogen | Y¹ is CH, Y² is |
| | chloro | | | CH, Y³ is CH |
| P.0008 | X¹ is chloro, X² is CH, X³ is | chlorodifluoromethyl | methoxy | Y¹ is CH, Y² is |
| | chloro | | | CH, Y³ is CH |
| P.0009 | X¹ is chloro, X² is CH, X³ is | chlorodifluoromethyl | methyl | Y¹ is CH, Y² is |
| | chloro | | | CH, Y³ is CH |
| P.0010 | X¹ is chloro, X² is CH, X³ is | chlorodifluoromethyl | nitro | Y¹ is CH, Y² is |
| | chloro | | | CH, Y³ is CH |
| P.0011 | X¹ is chloro, X² is CH, X³ is | chlorodifluoromethyl | trifluoromethoxy | Y¹ is CH, Y² is |
| | chloro | | | CH, Y³ is CH |
| P.0012 | X¹ is chloro, X² is CH, X³ is | chlorodifluoromethyl | trifluoromethyl | Y¹ is CH, Y² is |
| | chloro | | | CH, Y³ is CH |
| P.0013 | X¹ is chloro, X² is CH, X³ is | difluoromethyl | bromo | Y¹ is CH, Y² is |
| | chloro | | | CH, Y³ is CH |
| P.0014 | X¹ is chloro, X² is CH, X³ is | difluoromethyl | chloro | Y¹ is CH, Y² is |
| | chloro | | | CH, Y³ is CH |
| P.0015 | X¹ is chloro, X² is CH, X³ is | difluoromethyl | cyano | Y¹ is CH, Y² is |
| | chloro | | | CH, Y³ is CH |
| P.0016 | X¹ is chloro, X² is CH, X³ is | difluoromethyl | cyclopropyl | Y¹ is CH, Y² is |
| | chloro | | | CH, Y³ is CH |
| P.0017 | X¹ is chloro, X² is CH, X³ is | difluoromethyl | ethyl | Y¹ is CH, Y² is |
| | chloro | | | CH, Y³ is CH |
| P.0018 | X¹ is chloro, X² is CH, X³ is | difluoromethyl | fluoro | Y¹ is CH, Y² is |
| | chloro | | | CH, Y³ is CH |
| P.0019 | X¹ is chloro, X² is CH, X³ is | difluoromethyl | hydrogen | Y¹ is CH, Y² is |
| | chloro | | | CH, Y³ is CH |
| P.0020 | X¹ is chloro, X² is CH, X³ is | difluoromethyl | methoxy | Y¹ is CH, Y² is |
| | chloro | | | CH, Y³ is CH |
| P.0021 | X¹ is chloro, X² is CH, X³ is | difluoromethyl | methyl | Y¹ is CH, Y² is |
| | chloro | | | CH, Y³ is CH |
| P.0022 | X¹ is chloro, X² is CH, X³ is | difluoromethyl | nitro | Y¹ is CH, Y² is |
| | chloro | | | CH, Y³ is CH |
| P.0023 | X¹ is chloro, X² is CH, X³ is | difluoromethyl | trifluoromethoxy | Y¹ is CH, Y² is |
| | chloro | | | CH, Y³ is CH |
| P.0024 | X¹ is chloro, X² is CH, X³ is | difluoromethyl | trifluoromethyl | Y¹ is CH, Y² is |
| | chloro | | | CH, Y³ is CH |
| P.0025 | X¹ is chloro, X² is CH, X³ is | trifluoromethyl | bromo | Y¹ is CH, Y² is |
| | chloro | | | CH, Y³ is CH |
| P.0026 | X¹ is chloro, X² is CH, X³ is | trifluoromethyl | chloro | Y¹ is CH, Y² is |
| | chloro | | | CH, Y³ is CH |
| P.0027 | X¹ is chloro, X² is CH, X³ is | trifluoromethyl | cyano | Y¹ is CH, Y² is |
| | chloro | | | CH, Y³ is CH |
| P.0028 | X¹ is chloro, X² is CH, X³ is | trifluoromethyl | cyclopropyl | Y¹ is CH, Y² is |
| | chloro | | | CH, Y³ is CH |
| P.0029 | X¹ is chloro, X² is CH, X³ is | trifluoromethyl | ethyl | Y¹ is CH, Y² is |
| | chloro | | | CH, Y³ is CH |
| P.0030 | X¹ is chloro, X² is CH, X³ is | trifluoromethyl | fluoro | Y¹ is CH, Y² is |
| | chloro | | | CH, Y³ is CH |
| P.0031 | X¹ is chloro, X² is CH, X³ is | trifluoromethyl | hydrogen | Y¹ is CH, Y² is |
| | chloro | | | CH, Y³ is CH |
| P.0032 | X¹ is chloro, X² is CH, X³ is | trifluoromethyl | methoxy | Y¹ is CH, Y² is |
| | chloro | | | CH, Y³ is CH |
| P.0033 | X¹ is chloro, X² is CH, X³ is | trifluoromethyl | methyl | Y¹ is CH, Y² is |
| | chloro | | | CH, Y³ is CH |
| P.0034 | X¹ is chloro, X² is CH, X³ is | trifluoromethyl | nitro | Y¹ is CH, Y² is |
| | chloro | | | CH, Y³ is CH |
| P.0035 | X¹ is chloro, X² is CH, X³ is | trifluoromethyl | trifluoromethoxy | Y¹ is CH, Y² is |
| | chloro | | | CH, Y³ is CH |
| P.0036 | X¹ is chloro, X² is CH, X³ is | trifluoromethyl | trifluoromethyl | Y¹ is CH, Y² is |
| | chloro | | | CH, Y³ is CH |
| P.0037 | X¹ is chloro, X² is C—F, X³ is | chlorodifluoromethyl | bromo | Y¹ is CH, Y² is |
| | hydrogen | | | CH, Y³ is CH |
| P.0038 | X¹ is chloro, X² is C—F, X³ is | chlorodifluoromethyl | chloro | Y¹ is CH, Y² is |
| | hydrogen | | | CH, Y³ is CH |
| P.0039 | X¹ is chloro, X² is C—F, X³ is | chlorodifluoromethyl | cyano | Y¹ is CH, Y² is |
| | hydrogen | | | CH, Y³ is CH |
| P.0040 | X¹ is chloro, X² is C—F, X³ is | chlorodifluoromethyl | cyclopropyl | Y¹ is CH, Y² is |
| | hydrogen | | | CH, Y³ is CH |
| P.0041 | X¹ is chloro, X² is C—F, X³ is | chlorodifluoromethyl | ethyl | Y¹ is CH, Y² is |
| | hydrogen | | | CH, Y³ is CH |
| P.0042 | X¹ is chloro, X² is C—F, X³ is | chlorodifluoromethyl | fluoro | Y¹ is CH, Y² is |
| | hydrogen | | | CH, Y³ is CH |
| P.0043 | X¹ is chloro, X² is C—F, X³ is | chlorodifluoromethyl | hydrogen | Y¹ is CH, Y² is |
| | hydrogen | | | CH, Y³ is CH |
| P.0044 | X¹ is chloro, X² is C—F, X³ is | chlorodifluoromethyl | methoxy | Y¹ is CH, Y² is |
| | hydrogen | | | CH, Y³ is CH |
| P.0045 | X¹ is chloro, X² is C—F, X³ is | chlorodifluoromethyl | methyl | Y¹ is CH, Y² is |
| | hydrogen | | | CH, Y³ is CH |
| P.0046 | X¹ is chloro, X² is C—F, X³ is | chlorodifluoromethyl | nitro | Y¹ is CH, Y² is |
| | hydrogen | | | CH, Y³ is CH |
| P.0047 | X¹ is chloro, X² is C—F, X³ is | chlorodifluoromethyl | trifluoromethoxy | Y¹ is CH, Y² is |
| | hydrogen | | | CH, Y³ is CH |
| P.0048 | X¹ is chloro, X² is C—F, X³ is | chlorodifluoromethyl | trifluoromethyl | Y¹ is CH, Y² is |
| | hydrogen | | | CH, Y³ is CH |
| P.0049 | X¹ is chloro, X² is C—F, X³ is | difluoromethyl | bromo | Y¹ is CH, Y² is |
| | hydrogen | | | CH, Y³ is CH |
| P.0050 | X¹ is chloro, X² is C—F, X³ is | difluoromethyl | chloro | Y¹ is CH, Y² is |
| | hydrogen | | | CH, Y³ is CH |
| P.0051 | X¹ is chloro, X² is C—F, X³ is | difluoromethyl | cyano | Y¹ is CH, Y² is |
| | hydrogen | | | CH, Y³ is CH |
| P.0052 | X¹ is chloro, X² is C—F, X³ is | difluoromethyl | cyclopropyl | Y¹ is CH, Y² is |
| | hydrogen | | | CH, Y³ is CH |
| P.0053 | X¹ is chloro, X² is C—F, X³ is | difluoromethyl | ethyl | Y¹ is CH, Y² is |
| | hydrogen | | | CH, Y³ is CH |
| P.0054 | X¹ is chloro, X² is C—F, X³ is | difluoromethyl | fluoro | Y¹ is CH, Y² is |
| | hydrogen | | | CH, Y³ is CH |
| P.0055 | X¹ is chloro, X² is C—F, X³ is | difluoromethyl | hydrogen | Y¹ is CH, Y² is |
| | hydrogen | | | CH, Y³ is CH |
| P.0056 | X¹ is chloro, X² is C—F, X³ is | difluoromethyl | methoxy | Y¹ is CH, Y² is |
| | hydrogen | | | CH, Y³ is CH |
| P.0057 | X¹ is chloro, X² is C—F, X³ is | difluoromethyl | methyl | Y¹ is CH, Y² is |
| | hydrogen | | | CH, Y³ is CH |
| P.0058 | X¹ is chloro, X² is C—F, X³ is | difluoromethyl | nitro | Y¹ is CH, Y² is |
| | hydrogen | | | CH, Y³ is CH |
| P.0059 | X¹ is chloro, X² is C—F, X³ is | difluoromethyl | trifluoromethoxy | Y¹ is CH, Y² is |
| | hydrogen | | | CH, Y³ is CH |
| P.0060 | X¹ is chloro, X² is C—F, X³ is | difluoromethyl | trifluoromethyl | Y¹ is CH, Y² is |
| | hydrogen | | | CH, Y³ is CH |
| P.0061 | X¹ is chloro, X² is C—F, X³ is | trifluoromethyl | bromo | Y¹ is CH, Y² is |
| | hydrogen | | | CH, Y³ is CH |
| P.0062 | X¹ is chloro, X² is C—F, X³ is | trifluoromethyl | chloro | Y¹ is CH, Y² is |
| | hydrogen | | | CH, Y³ is CH |
| P.0063 | X¹ is chloro, X² is C—F, X³ is | trifluoromethyl | cyano | Y¹ is CH, Y² is |
| | hydrogen | | | CH, Y³ is CH |
| P.0064 | X¹ is chloro, X² is C—F, X³ is | trifluoromethyl | cyclopropyl | Y¹ is CH, Y² is |
| | hydrogen | | | CH, Y³ is CH |
| P.0065 | X¹ is chloro, X² is C—F, X³ is | trifluoromethyl | ethyl | Y¹ is CH, Y² is |
| | hydrogen | | | CH, Y³ is CH |
| P.0066 | X¹ is chloro, X² is C—F, X³ is | trifluoromethyl | fluoro | Y¹ is CH, Y² is |
| | hydrogen | | | CH, Y³ is CH |
| P.0067 | X¹ is chloro, X² is C—F, X³ is | trifluoromethyl | hydrogen | Y¹ is CH, Y² is |
| | hydrogen | | | CH, Y³ is CH |
| P.0068 | X¹ is chloro, X² is C—F, X³ is | trifluoromethyl | methoxy | Y¹ is CH, Y² is |
| | hydrogen | | | CH, Y³ is CH |
| P.0069 | X¹ is chloro, X² is C—F, X³ is | trifluoromethyl | methyl | Y¹ is CH, Y² is |
| | hydrogen | | | CH, Y³ is CH |
| P.0070 | X¹ is chloro, X² is C—F, X³ is | trifluoromethyl | nitro | Y¹ is CH, Y² is |
| | hydrogen | | | CH, Y³ is CH |
| P.0071 | X¹ is chloro, X² is C—F, X³ is | trifluoromethyl | trifluoromethoxy | Y¹ is CH, Y² is |
| | hydrogen | | | CH, Y³ is CH |
| P.0072 | X¹ is chloro, X² is C—F, X³ is | trifluoromethyl | trifluoromethyl | Y¹ is CH, Y² is |
| | hydrogen | | | CH, Y³ is CH |
| P.0073 | X¹ is fluoro, X² is C—Cl, X³ | chlorodifluoromethyl | bromo | Y¹ is CH, Y² is |
| | is hydrogen | | | CH, Y³ is CH |
| P.0074 | X¹ is fluoro, X² is C—Cl, X³ | chlorodifluoromethyl | chloro | Y¹ is CH, Y² is |
| | is hydrogen | | | CH, Y³ is CH |
| P.0075 | X¹ is fluoro, X² is C—Cl, X³ | chlorodifluoromethyl | cyano | Y¹ is CH, Y² is |
| | is hydrogen | | | CH, Y³ is CH |
| P.0076 | X¹ is fluoro, X² is C—Cl, X³ | chlorodifluoromethyl | cyclopropyl | Y¹ is CH, Y² is |
| | is hydrogen | | | CH, Y³ is CH |
| P.0077 | X¹ is fluoro, X² is C—Cl, X³ | chlorodifluoromethyl | ethyl | Y¹ is CH, Y² is |
| | is hydrogen | | | CH, Y³ is CH |
| P.0078 | X¹ is fluoro, X² is C—Cl, X³ | chlorodifluoromethyl | fluoro | Y¹ is CH, Y² is |
| | is hydrogen | | | CH, Y³ is CH |
| P.0079 | X¹ is fluoro, X² is C—Cl, X³ | chlorodifluoromethyl | hydrogen | Y¹ is CH, Y² is |
| | is hydrogen | | | CH, Y³ is CH |
| P.0080 | X¹ is fluoro, X² is C—Cl, X³ | chlorodifluoromethyl | methoxy | Y¹ is CH, Y² is |
| | is hydrogen | | | CH, Y³ is CH |
| P.0081 | X¹ is fluoro, X² is C—Cl, X³ | chlorodifluoromethyl | methyl | Y¹ is CH, Y² is |
| | is hydrogen | | | CH, Y³ is CH |
| P.0082 | X¹ is fluoro, X² is C—Cl, X³ | chlorodifluoromethyl | nitro | Y¹ is CH, Y² is |
| | is hydrogen | | | CH, Y³ is CH |
| P.0083 | X¹ is fluoro, X² is C—Cl, X³ | chlorodifluoromethyl | trifluoromethoxy | Y¹ is CH, Y² is |
| | is hydrogen | | | CH, Y³ is CH |
| P.0084 | X¹ is fluoro, X² is C—Cl, X³ | chlorodifluoromethyl | trifluoromethyl | Y¹ is CH, Y² is |
| | is hydrogen | | | CH, Y³ is CH |
| P.0085 | X¹ is fluoro, X² is C—Cl, X³ | difluoromethyl | bromo | Y¹ is CH, Y² is |
| | is hydrogen | | | CH, Y³ is CH |
| P.0086 | X¹ is fluoro, X² is C—Cl, X³ | difluoromethyl | chloro | Y¹ is CH, Y² is |
| | is hydrogen | | | CH, Y³ is CH |
| P.0087 | X¹ is fluoro, X² is C—Cl, X³ | difluoromethyl | cyano | Y¹ is CH, Y² is |
| | is hydrogen | | | CH, Y³ is CH |
| P.0088 | X¹ is fluoro, X² is C—Cl, X³ | difluoromethyl | cyclopropyl | Y¹ is CH, Y² is |
| | is hydrogen | | | CH, Y³ is CH |
| P.0089 | X¹ is fluoro, X² is C—Cl, X³ | difluoromethyl | ethyl | Y¹ is CH, Y² is |
| | is hydrogen | | | CH, Y³ is CH |
| P.0090 | X¹ is fluoro, X² is C—Cl, X³ | difluoromethyl | fluoro | Y¹ is CH, Y² is |
| | is hydrogen | | | CH, Y³ is CH |
| P.0091 | X¹ is fluoro, X² is C—Cl, X³ | difluoromethyl | hydrogen | Y¹ is CH, Y² is |
| | is hydrogen | | | CH, Y³ is CH |
| P.0092 | X¹ is fluoro, X² is C—Cl, X³ | difluoromethyl | methoxy | Y¹ is CH, Y² is |
| | is hydrogen | | | CH, Y³ is CH |
| P.0093 | X¹ is fluoro, X² is C—Cl, X³ | difluoromethyl | methyl | Y¹ is CH, Y² is |
| | is hydrogen | | | CH, Y³ is CH |
| P.0094 | X¹ is fluoro, X² is C—Cl, X³ | difluoromethyl | nitro | Y¹ is CH, Y² is |
| | is hydrogen | | | CH, Y³ is CH |
| P.0095 | X¹ is fluoro, X² is C—Cl, X³ | difluoromethyl | trifluoromethoxy | Y¹ is CH, Y² is |
| | is hydrogen | | | CH, Y³ is CH |
| P.0096 | X¹ is fluoro, X² is C—Cl, X³ | difluoromethyl | trifluoromethyl | Y¹ is CH, Y² is |
| | is hydrogen | | | CH, Y³ is CH |
| P.0097 | X¹ is fluoro, X² is C—Cl, X³ | trifluoromethyl | bromo | Y¹ is CH, Y² is |
| | is hydrogen | | | CH, Y³ is CH |
| P.0098 | X¹ is fluoro, X² is C—Cl, X³ | trifluoromethyl | chloro | Y¹ is CH, Y² is |
| | is hydrogen | | | CH, Y³ is CH |
| P.0099 | X¹ is fluoro, X² is C—Cl, X³ | trifluoromethyl | cyano | Y¹ is CH, Y² is |
| | is hydrogen | | | CH, Y³ is CH |
| P.0100 | X¹ is fluoro, X² is C—Cl, X³ | trifluoromethyl | cyclopropyl | Y¹ is CH, Y² is |
| | is hydrogen | | | CH, Y³ is CH |
| P.0101 | X¹ is fluoro, X² is C—Cl, X³ | trifluoromethyl | ethyl | Y¹ is CH, Y² is |
| | is hydrogen | | | CH, Y³ is CH |
| P.0102 | X¹ is fluoro, X² is C—Cl, X³ | trifluoromethyl | fluoro | Y¹ is CH, Y² is |
| | is hydrogen | | | CH, Y³ is CH |
| P.0103 | X¹ is fluoro, X² is C—Cl, X³ | trifluoromethyl | hydrogen | Y¹ is CH, Y² is |
| | is hydrogen | | | CH, Y³ is CH |
| P.0104 | X¹ is fluoro, X² is C—Cl, X³ | trifluoromethyl | methoxy | Y¹ is CH, Y² is |
| | is hydrogen | | | CH, Y³ is CH |
| P.0105 | X¹ is fluoro, X² is C—Cl, X³ | trifluoromethyl | methyl | Y¹ is CH, Y² is |
| | is hydrogen | | | CH, Y³ is CH |
| P.0106 | X¹ is fluoro, X² is C—Cl, X³ | trifluoromethyl | nitro | Y¹ is CH, Y² is |
| | is hydrogen | | | CH, Y³ is CH |
| P.0107 | X¹ is fluoro, X² is C—Cl, X³ | trifluoromethyl | trifluoromethoxy | Y¹ is CH, Y² is |
| | is hydrogen | | | CH, Y³ is CH |
| P.0108 | X¹ is fluoro, X² is C—Cl, X³ | trifluoromethyl | trifluoromethyl | Y¹ is CH, Y² is |
| | is hydrogen | | | CH, Y³ is CH |
| P.0109 | X¹ is chloro, X² is C—Cl, X³ | chlorodifluoromethyl | bromo | Y¹ is CH, Y² is |
| | is hydrogen | | | CH, Y³ is CH |
| P.0110 | X¹ is chloro, X² is C—Cl, X³ | chlorodifluoromethyl | chloro | Y¹ is CH, Y² is |
| | is hydrogen | | | CH, Y³ is CH |
| P.0111 | X¹ is chloro, X² is C—Cl, X³ | chlorodifluoromethyl | cyano | Y¹ is CH, Y² is |
| | is hydrogen | | | CH, Y³ is CH |
| P.0112 | X¹ is chloro, X² is C—Cl, X³ | chlorodifluoromethyl | cyclopropyl | Y¹ is CH, Y² is |
| | is hydrogen | | | CH, Y³ is CH |
| P.0113 | X¹ is chloro, X² is C—Cl, X³ | chlorodifluoromethyl | ethyl | Y¹ is CH, Y² is |
| | is hydrogen | | | CH, Y³ is CH |
| P.0114 | X¹ is chloro, X² is C—Cl, X³ | chlorodifluoromethyl | fluoro | Y¹ is CH, Y² is |
| | is hydrogen | | | CH, Y³ is CH |
| P.0115 | X¹ is chloro, X² is C—Cl, X³ | chlorodifluoromethyl | hydrogen | Y¹ is CH, Y² is |
| | is hydrogen | | | CH, Y³ is CH |
| P.0116 | X¹ is chloro, X² is C—Cl, X³ | chlorodifluoromethyl | methoxy | Y¹ is CH, Y² is |
| | is hydrogen | | | CH, Y³ is CH |
| P.0117 | X¹ is chloro, X² is C—Cl, X³ | chlorodifluoromethyl | methyl | Y¹ is CH, Y² is |
| | is hydrogen | | | CH, Y³ is CH |
| P.0118 | X¹ is chloro, X² is C—Cl, X³ | chlorodifluoromethyl | nitro | Y¹ is CH, Y² is |
| | is hydrogen | | | CH, Y³ is CH |
| P.0119 | X¹ is chloro, X² is C—Cl, X³ | chlorodifluoromethyl | trifluoromethoxy | Y¹ is CH, Y² is |
| | is hydrogen | | | CH, Y³ is CH |
| P.0120 | X¹ is chloro, X² is C—Cl, X³ | chlorodifluoromethyl | trifluoromethyl | Y¹ is CH, Y² is |
| | is hydrogen | | | CH, Y³ is CH |
| P.0121 | X¹ is chloro, X² is C—Cl, X³ | difluoromethyl | bromo | Y¹ is CH, Y² is |
| | is hydrogen | | | CH, Y³ is CH |
| P.0122 | X¹ is chloro, X² is C—Cl, X³ | difluoromethyl | chloro | Y¹ is CH, Y² is |
| | is hydrogen | | | CH, Y³ is CH |
| P.0123 | X¹ is chloro, X² is C—Cl, X³ | difluoromethyl | cyano | Y¹ is CH, Y² is |
| | is hydrogen | | | CH, Y³ is CH |
| P.0124 | X¹ is chloro, X² is C—Cl, X³ | difluoromethyl | cyclopropyl | Y¹ is CH, Y² is |
| | is hydrogen | | | CH, Y³ is CH |
| P.0125 | X¹ is chloro, X² is C—Cl, X³ | difluoromethyl | ethyl | Y¹ is CH, Y² is |
| | is hydrogen | | | CH, Y³ is CH |
| P.0126 | X¹ is chloro, X² is C—Cl, X³ | difluoromethyl | fluoro | Y¹ is CH, Y² is |
| | is hydrogen | | | CH, Y³ is CH |
| P.0127 | X¹ is chloro, X² is C—Cl, X³ | difluoromethyl | hydrogen | Y¹ is CH, Y² is |
| | is hydrogen | | | CH, Y³ is CH |
| P.0128 | X¹ is chloro, X² is C—Cl, X³ | difluoromethyl | methoxy | Y¹ is CH, Y² is |
| | is hydrogen | | | CH, Y³ is CH |
| P.0129 | X¹ is chloro, X² is C—Cl, X³ | difluoromethyl | methyl | Y¹ is CH, Y² is |
| | is hydrogen | | | CH, Y³ is CH |
| P.0130 | X¹ is chloro, X² is C—Cl, X³ | difluoromethyl | nitro | Y¹ is CH, Y² is |
| | is hydrogen | | | CH, Y³ is CH |
| P.0131 | X¹ is chloro, X² is C—Cl, X³ | difluoromethyl | trifluoromethoxy | Y¹ is CH, Y² is |
| | is hydrogen | | | CH, Y³ is CH |
| P.0132 | X¹ is chloro, X² is C—Cl, X³ | difluoromethyl | trifluoromethyl | Y¹ is CH, Y² is |
| | is hydrogen | | | CH, Y³ is CH |
| P.0133 | X¹ is chloro, X² is C—Cl, X³ | trifluoromethyl | bromo | Y¹ is CH, Y² is |
| | is hydrogen | | | CH, Y³ is CH |
| P.0134 | X¹ is chloro, X² is C—Cl, X³ | trifluoromethyl | chloro | Y¹ is CH, Y² is |
| | is hydrogen | | | CH, Y³ is CH |
| P.0135 | X¹ is chloro, X² is C—Cl, X³ | trifluoromethyl | cyano | Y¹ is CH, Y² is |
| | is hydrogen | | | CH, Y³ is CH |
| P.0136 | X¹ is chloro, X² is C—Cl, X³ | trifluoromethyl | cyclopropyl | Y¹ is CH, Y² is |
| | is hydrogen | | | CH, Y³ is CH |
| P.0137 | X¹ is chloro, X² is C—Cl, X³ | trifluoromethyl | ethyl | Y¹ is CH, Y² is |
| | is hydrogen | | | CH, Y³ is CH |
| P.0138 | X¹ is chloro, X² is C—Cl, X³ | trifluoromethyl | fluoro | Y¹ is CH, Y² is |
| | is hydrogen | | | CH, Y³ is CH |
| P.0139 | X¹ is chloro, X² is C—Cl, X³ | trifluoromethyl | hydrogen | Y¹ is CH, Y² is |
| | is hydrogen | | | CH, Y³ is CH |
| P.0140 | X¹ is chloro, X² is C—Cl, X³ | trifluoromethyl | methoxy | Y¹ is CH, Y² is |
| | is hydrogen | | | CH, Y³ is CH |
| P.0141 | X¹ is chloro, X² is C—Cl, X³ | trifluoromethyl | methyl | Y¹ is CH, Y² is |
| | is hydrogen | | | CH, Y³ is CH |
| P.0142 | X¹ is chloro, X² is C—Cl, X³ | trifluoromethyl | nitro | Y¹ is CH, Y² is |
| | is hydrogen | | | CH, Y³ is CH |
| P.0143 | X¹ is chloro, X² is C—Cl, X³ | trifluoromethyl | trifluoromethoxy | Y¹ is CH, Y² is |
| | is hydrogen | | | CH, Y³ is CH |
| P.0144 | X¹ is chloro, X² is C—Cl, X³ | trifluoromethyl | trifluoromethyl | Y¹ is CH, Y² is |
| | is hydrogen | | | CH, Y³ is CH |
| P.0145 | X¹ is chloro, X² is C—Br, X³ | chlorodifluoromethyl | bromo | Y¹ is CH, Y² is |
| | is chloro | | | CH, Y³ is CH |
| P.0146 | X¹ is chloro, X² is C—Br, X³ | chlorodifluoromethyl | chloro | Y¹ is CH, Y² is |
| | is chloro | | | CH, Y³ is CH |
| P.0147 | X¹ is chloro, X² is C—Br, X³ | chlorodifluoromethyl | cyano | Y¹ is CH, Y² is |
| | is chloro | | | CH, Y³ is CH |
| P.0148 | X¹ is chloro, X² is C—Br, X³ | chlorodifluoromethyl | cyclopropyl | Y¹ is CH, Y² is |
| | is chloro | | | CH, Y³ is CH |
| P.0149 | X¹ is chloro, X² is C—Br, X³ | chlorodifluoromethyl | ethyl | Y¹ is CH, Y² is |
| | is chloro | | | CH, Y³ is CH |
| P.0150 | X¹ is chloro, X² is C—Br, X³ | chlorodifluoromethyl | fluoro | Y¹ is CH, Y² is |
| | is chloro | | | CH, Y³ is CH |
| P.0151 | X¹ is chloro, X² is C—Br, X³ | chlorodifluoromethyl | hydrogen | Y¹ is CH, Y² is |
| | is chloro | | | CH, Y³ is CH |
| P.0152 | X¹ is chloro, X² is C—Br, X³ | chlorodifluoromethyl | methoxy | Y¹ is CH, Y² is |
| | is chloro | | | CH, Y³ is CH |
| P.0153 | X¹ is chloro, X² is C—Br, X³ | chlorodifluoromethyl | methyl | Y¹ is CH, Y² is |
| | is chloro | | | CH, Y³ is CH |
| P.0154 | X¹ is chloro, X² is C—Br, X³ | chlorodifluoromethyl | nitro | Y¹ is CH, Y² is |
| | is chloro | | | CH, Y³ is CH |
| P.0155 | X¹ is chloro, X² is C—Br, X³ | chlorodifluoromethyl | trifluoromethoxy | Y¹ is CH, Y² is |
| | is chloro | | | CH, Y³ is CH |
| P.0156 | X¹ is chloro, X² is C—Br, X³ | chlorodifluoromethyl | trifluoromethyl | Y¹ is CH, Y² is |
| | is chloro | | | CH, Y³ is CH |
| P.0157 | X¹ is chloro, X² is C—Br, X³ | difluoromethyl | bromo | Y¹ is CH, Y² is |
| | is chloro | | | CH, Y³ is CH |
| P.0158 | X¹ is chloro, X² is C—Br, X³ | difluoromethyl | chloro | Y¹ is CH, Y² is |
| | is chloro | | | CH, Y³ is CH |
| P.0159 | X¹ is chloro, X² is C—Br, X³ | difluoromethyl | cyano | Y¹ is CH, Y² is |
| | is chloro | | | CH, Y³ is CH |
| P.0160 | X¹ is chloro, X² is C—Br, X³ | difluoromethyl | cyclopropyl | Y¹ is CH, Y² is |
| | is chloro | | | CH, Y³ is CH |
| P.0161 | X¹ is chloro, X² is C—Br, X³ | difluoromethyl | ethyl | Y¹ is CH, Y² is |
| | is chloro | | | CH, Y³ is CH |
| P.0162 | X¹ is chloro, X² is C—Br, X³ | difluoromethyl | fluoro | Y¹ is CH, Y² is |
| | is chloro | | | CH, Y³ is CH |
| P.0163 | X¹ is chloro, X² is C—Br, X³ | difluoromethyl | hydrogen | Y¹ is CH, Y² is |
| | is chloro | | | CH, Y³ is CH |
| P.0164 | X¹ is chloro, X² is C—Br, X³ | difluoromethyl | methoxy | Y¹ is CH, Y² is |
| | is chloro | | | CH, Y³ is CH |
| P.0165 | X¹ is chloro, X² is C—Br, X³ | difluoromethyl | methyl | Y¹ is CH, Y² is |
| | is chloro | | | CH, Y³ is CH |
| P.0166 | X¹ is chloro, X² is C—Br, X³ | difluoromethyl | nitro | Y¹ is CH, Y² is |
| | is chloro | | | CH, Y³ is CH |
| P.0167 | X¹ is chloro, X² is C—Br, X³ | difluoromethyl | trifluoromethoxy | Y¹ is CH, Y² is |
| | is chloro | | | CH, Y³ is CH |
| P.0168 | X¹ is chloro, X² is C—Br, X³ | difluoromethyl | trifluoromethyl | Y¹ is CH, Y² is |
| | is chloro | | | CH, Y³ is CH |
| P.0169 | X¹ is chloro, X² is C—Br, X³ | trifluoromethyl | bromo | Y¹ is CH, Y² is |
| | is chloro | | | CH, Y³ is CH |
| P.0170 | X¹ is chloro, X² is C—Br, X³ | trifluoromethyl | chloro | Y¹ is CH, Y² is |
| | is chloro | | | CH, Y³ is CH |
| P.0171 | X¹ is chloro, X² is C—Br, X³ | trifluoromethyl | cyano | Y¹ is CH, Y² is |
| | is chloro | | | CH, Y³ is CH |
| P.0172 | X¹ is chloro, X² is C—Br, X³ | trifluoromethyl | cyclopropyl | Y¹ is CH, Y² is |
| | is chloro | | | CH, Y³ is CH |
| P.0173 | X¹ is chloro, X² is C—Br, X³ | trifluoromethyl | ethyl | Y¹ is CH, Y² is |
| | is chloro | | | CH, Y³ is CH |
| P.0174 | X¹ is chloro, X² is C—Br, X³ | trifluoromethyl | fluoro | Y¹ is CH, Y² is |
| | is chloro | | | CH, Y³ is CH |
| P.0175 | X¹ is chloro, X² is C—Br, X³ | trifluoromethyl | hydrogen | Y¹ is CH, Y² is |
| | is chloro | | | CH, Y³ is CH |
| P.0176 | X¹ is chloro, X² is C—Br, X³ | trifluoromethyl | methoxy | Y¹ is CH, Y² is |
| | is chloro | | | CH, Y³ is CH |
| P.0177 | X¹ is chloro, X² is C—Br, X³ | trifluoromethyl | methyl | Y¹ is CH, Y² is |
| | is chloro | | | CH, Y³ is CH |
| P.0178 | X¹ is chloro, X² is C—Br, X³ | trifluoromethyl | nitro | Y¹ is CH, Y² is |
| | is chloro | | | CH, Y³ is CH |
| P.0179 | X¹ is chloro, X² is C—Br, X³ | trifluoromethyl | trifluoromethoxy | Y¹ is CH, Y² is |
| | is chloro | | | CH, Y³ is CH |
| P.0180 | X¹ is chloro, X² is C—Br, X³ | trifluoromethyl | trifluoromethyl | Y¹ is CH, Y² is |
| | is chloro | | | CH, Y³ is CH |
| P.0181 | X¹ is chloro, X² is C—F, X³ is | chlorodifluoromethyl | bromo | Y¹ is CH, Y² is |
| | chloro | | | CH, Y³ is CH |
| P.0182 | X¹ is chloro, X² is C—F, X³ is | chlorodifluoromethyl | chloro | Y¹ is CH, Y² is |
| | chloro | | | CH, Y³ is CH |
| P.0183 | X¹ is chloro, X² is C—F, X³ is | chlorodifluoromethyl | cyano | Y¹ is CH, Y² is |
| | chloro | | | CH, Y³ is CH |
| P.0184 | X¹ is chloro, X² is C—F, X³ is | chlorodifluoromethyl | cyclopropyl | Y¹ is CH, Y² is |
| | chloro | | | CH, Y³ is CH |
| P.0185 | X¹ is chloro, X² is C—F, X³ is | chlorodifluoromethyl | ethyl | Y¹ is CH, Y² is |
| | chloro | | | CH, Y³ is CH |
| P.0186 | X¹ is chloro, X² is C—F, X³ is | chlorodifluoromethyl | fluoro | Y¹ is CH, Y² is |
| | chloro | | | CH, Y³ is CH |
| P.0187 | X¹ is chloro, X² is C—F, X³ is | chlorodifluoromethyl | hydrogen | Y¹ is CH, Y² is |
| | chloro | | | CH, Y³ is CH |
| P.0188 | X¹ is chloro, X² is C—F, X³ is | chlorodifluoromethyl | methoxy | Y¹ is CH, Y² is |
| | chloro | | | CH, Y³ is CH |
| P.0189 | X¹ is chloro, X² is C—F, X³ is | chlorodifluoromethyl | methyl | Y¹ is CH, Y² is |
| | chloro | | | CH, Y³ is CH |
| P.0190 | X¹ is chloro, X² is C—F, X³ is | chlorodifluoromethyl | nitro | Y¹ is CH, Y² is |
| | chloro | | | CH, Y³ is CH |
| P.0191 | X¹ is chloro, X² is C—F, X³ is | chlorodifluoromethyl | trifluoromethoxy | Y¹ is CH, Y² is |
| | chloro | | | CH, Y³ is CH |
| P.0192 | X¹ is chloro, X² is C—F, X³ is | chlorodifluoromethyl | trifluoromethyl | Y¹ is CH, Y² is |
| | chloro | | | CH, Y³ is CH |
| P.0193 | X¹ is chloro, X² is C—F, X³ is | difluoromethyl | bromo | Y¹ is CH, Y² is |
| | chloro | | | CH, Y³ is CH |
| P.0194 | X¹ is chloro, X² is C—F, X³ is | difluoromethyl | chloro | Y¹ is CH, Y² is |
| | chloro | | | CH, Y³ is CH |
| P.0195 | X¹ is chloro, X² is C—F, X³ is | difluoromethyl | cyano | Y¹ is CH, Y² is |
| | chloro | | | CH, Y³ is CH |
| P.0196 | X¹ is chloro, X² is C—F, X³ is | difluoromethyl | cyclopropyl | Y¹ is CH, Y² is |
| | chloro | | | CH, Y³ is CH |
| P.0197 | X¹ is chloro, X² is C—F, X³ is | difluoromethyl | ethyl | Y¹ is CH, Y² is |
| | chloro | | | CH, Y³ is CH |
| P.0198 | X¹ is chloro, X² is C—F, X³ is | difluoromethyl | fluoro | Y¹ is CH, Y² is |
| | chloro | | | CH, Y³ is CH |
| P.0199 | X¹ is chloro, X² is C—F, X³ is | difluoromethyl | hydrogen | Y¹ is CH, Y² is |
| | chloro | | | CH, Y³ is CH |
| P.0200 | X¹ is chloro, X² is C—F, X³ is | difluoromethyl | methoxy | Y¹ is CH, Y² is |
| | chloro | | | CH, Y³ is CH |
| P.0201 | X¹ is chloro, X² is C—F, X³ is | difluoromethyl | methyl | Y¹ is CH, Y² is |
| | chloro | | | CH, Y³ is CH |
| P.0202 | X¹ is chloro, X² is C—F, X³ is | difluoromethyl | nitro | Y¹ is CH, Y² is |
| | chloro | | | CH, Y³ is CH |
| P.0203 | X¹ is chloro, X² is C—F, X³ is | difluoromethyl | trifluoromethoxy | Y¹ is CH, Y² is |
| | chloro | | | CH, Y³ is CH |
| P.0204 | X¹ is chloro, X² is C—F, X³ is | difluoromethyl | trifluoromethyl | Y¹ is CH, Y² is |
| | chloro | | | CH, Y³ is CH |
| P.0205 | X¹ is chloro, X² is C—F, X³ is | trifluoromethyl | bromo | Y¹ is CH, Y² is |
| | chloro | | | CH, Y³ is CH |
| P.0206 | X¹ is chloro, X² is C—F, X³ is | trifluoromethyl | chloro | Y¹ is CH, Y² is |
| | chloro | | | CH, Y³ is CH |
| P.0207 | X¹ is chloro, X² is C—F, X³ is | trifluoromethyl | cyano | Y¹ is CH, Y² is |
| | chloro | | | CH, Y³ is CH |
| P.0208 | X¹ is chloro, X² is C—F, X³ is | trifluoromethyl | cyclopropyl | Y¹ is CH, Y² is |
| | chloro | | | CH, Y³ is CH |
| P.0209 | X¹ is chloro, X² is C—F, X³ is | trifluoromethyl | ethyl | Y¹ is CH, Y² is |
| | chloro | | | CH, Y³ is CH |
| P.0210 | X¹ is chloro, X² is C—F, X³ is | trifluoromethyl | fluoro | Y¹ is CH, Y² is |
| | chloro | | | CH, Y³ is CH |
| P.0211 | X¹ is chloro, X² is C—F, X³ is | trifluoromethyl | hydrogen | Y¹ is CH, Y² is |
| | chloro | | | CH, Y³ is CH |
| P.0212 | X¹ is chloro, X² is C—F, X³ is | trifluoromethyl | methoxy | Y¹ is CH, Y² is |
| | chloro | | | CH, Y³ is CH |
| P.0213 | X¹ is chloro, X² is C—F, X³ is | trifluoromethyl | methyl | Y¹ is CH, Y² is |
| | chloro | | | CH, Y³ is CH |
| P.0214 | X¹ is chloro, X² is C—F, X³ is | trifluoromethyl | nitro | Y¹ is CH, Y² is |
| | chloro | | | CH, Y³ is CH |
| P.0215 | X¹ is chloro, X² is C—F, X³ is | trifluoromethyl | trifluoromethoxy | Y¹ is CH, Y² is |
| | chloro | | | CH, Y³ is CH |
| P.0216 | X¹ is chloro, X² is C—F, X³ is | trifluoromethyl | trifluoromethyl | Y¹ is CH, Y² is |
| | chloro | | | CH, Y³ is CH |
| P.0217 | X¹ is chloro, X² is C—Cl, X³ | chlorodifluoromethyl | bromo | Y¹ is CH, Y² is |
| | is chloro | | | CH, Y³ is CH |
| P.0218 | X¹ is chloro, X² is C—Cl, X³ | chlorodifluoromethyl | chloro | Y¹ is CH, Y² is |
| | is chloro | | | CH, Y³ is CH |
| P.0219 | X¹ is chloro, X² is C—Cl, X³ | chlorodifluoromethyl | cyano | Y¹ is CH, Y² is |
| | is chloro | | | CH, Y³ is CH |
| P.0220 | X¹ is chloro, X² is C—Cl, X³ | chlorodifluoromethyl | cyclopropyl | Y¹ is CH, Y² is |
| | is chloro | | | CH, Y³ is CH |
| P.0221 | X¹ is chloro, X² is C—Cl, X³ | chlorodifluoromethyl | ethyl | Y¹ is CH, Y² is |
| | is chloro | | | CH, Y³ is CH |
| P.0222 | X¹ is chloro, X² is C—Cl, X³ | chlorodifluoromethyl | fluoro | Y¹ is CH, Y² is |
| | is chloro | | | CH, Y³ is CH |
| P.0223 | X¹ is chloro, X² is C—Cl, X³ | chlorodifluoromethyl | hydrogen | Y¹ is CH, Y² is |
| | is chloro | | | CH, Y³ is CH |
| P.0224 | X¹ is chloro, X² is C—Cl, X³ | chlorodifluoromethyl | methoxy | Y¹ is CH, Y² is |
| | is chloro | | | CH, Y³ is CH |
| P.0225 | X¹ is chloro, X² is C—Cl, X³ | chlorodifluoromethyl | methyl | Y¹ is CH, Y² is |
| | is chloro | | | CH, Y³ is CH |
| P.0226 | X¹ is chloro, X² is C—Cl, X³ | chlorodifluoromethyl | nitro | Y¹ is CH, Y² is |
| | is chloro | | | CH, Y³ is CH |
| P.0227 | X¹ is chloro, X² is C—Cl, X³ | chlorodifluoromethyl | trifluoromethoxy | Y¹ is CH, Y² is |
| | is chloro | | | CH, Y³ is CH |
| P.0228 | X¹ is chloro, X² is C—Cl, X³ | chlorodifluoromethyl | trifluoromethyl | Y¹ is CH, Y² is |
| | is chloro | | | CH, Y³ is CH |
| P.0229 | X¹ is chloro, X² is C—Cl, X³ | difluoromethyl | bromo | Y¹ is CH, Y² is |
| | is chloro | | | CH, Y³ is CH |
| P.0230 | X¹ is chloro, X² is C—Cl, X³ | difluoromethyl | chloro | Y¹ is CH, Y² is |
| | is chloro | | | CH, Y³ is CH |
| P.0231 | X¹ is chloro, X² is C—Cl, X³ | difluoromethyl | cyano | Y¹ is CH, Y² is |
| | is chloro | | | CH, Y³ is CH |
| P.0232 | X¹ is chloro, X² is C—Cl, X³ | difluoromethyl | cyclopropyl | Y¹ is CH, Y² is |
| | is chloro | | | CH, Y³ is CH |
| P.0233 | X¹ is chloro, X² is C—Cl, X³ | difluoromethyl | ethyl | Y¹ is CH, Y² is |
| | is chloro | | | CH, Y³ is CH |
| P.0234 | X¹ is chloro, X² is C—Cl, X³ | difluoromethyl | fluoro | Y¹ is CH, Y² is |
| | is chloro | | | CH, Y³ is CH |
| P.0235 | X¹ is chloro, X² is C—Cl, X³ | difluoromethyl | hydrogen | Y¹ is CH, Y² is |
| | is chloro | | | CH, Y³ is CH |
| P.0236 | X¹ is chloro, X² is C—Cl, X³ | difluoromethyl | methoxy | Y¹ is CH, Y² is |
| | is chloro | | | CH, Y³ is CH |
| P.0237 | X¹ is chloro, X² is C—Cl, X³ | difluoromethyl | methyl | Y¹ is CH, Y² is |
| | is chloro | | | CH, Y³ is CH |
| P.0238 | X¹ is chloro, X² is C—Cl, X³ | difluoromethyl | nitro | Y¹ is CH, Y² is |
| | is chloro | | | CH, Y³ is CH |
| P.0239 | X¹ is chloro, X² is C—Cl, X³ | difluoromethyl | trifluoromethoxy | Y¹ is CH, Y² is |
| | is chloro | | | CH, Y³ is CH |
| P.0240 | X¹ is chloro, X² is C—Cl, X³ | difluoromethyl | trifluoromethyl | Y¹ is CH, Y² is |
| | is chloro | | | CH, Y³ is CH |
| P.0241 | X¹ is chloro, X² is C—Cl, X³ | trifluoromethyl | bromo | Y¹ is CH, Y² is |
| | is chloro | | | CH, Y³ is CH |
| P.0242 | X¹ is chloro, X² is C—Cl, X³ | trifluoromethyl | chloro | Y¹ is CH, Y² is |
| | is chloro | | | CH, Y³ is CH |
| P.0243 | X¹ is chloro, X² is C—Cl, X³ | trifluoromethyl | cyano | Y¹ is CH, Y² is |
| | is chloro | | | CH, Y³ is CH |
| P.0244 | X¹ is chloro, X² is C—Cl, X³ | trifluoromethyl | cyclopropyl | Y¹ is CH, Y² is |
| | is chloro | | | CH, Y³ is CH |
| P.0245 | X¹ is chloro, X² is C—Cl, X³ | trifluoromethyl | ethyl | Y¹ is CH, Y² is |
| | is chloro | | | CH, Y³ is CH |
| P.0246 | X¹ is chloro, X² is C—Cl, X³ | trifluoromethyl | fluoro | Y¹ is CH, Y² is |
| | is chloro | | | CH, Y³ is CH |
| P.0247 | X¹ is chloro, X² is C—Cl, X³ | trifluoromethyl | hydrogen | Y¹ is CH, Y² is |
| | is chloro | | | CH, Y³ is CH |
| P.0248 | X¹ is chloro, X² is C—Cl, X³ | trifluoromethyl | methoxy | Y¹ is CH, Y² is |
| | is chloro | | | CH, Y³ is CH |
| P.0249 | X¹ is chloro, X² is C—Cl, X³ | trifluoromethyl | methyl | Y¹ is CH, Y² is |
| | is chloro | | | CH, Y³ is CH |
| P.0250 | X¹ is chloro, X² is C—Cl, X³ | trifluoromethyl | nitro | Y¹ is CH, Y² is |
| | is chloro | | | CH, Y³ is CH |
| P.0251 | X¹ is chloro, X² is C—Cl, X³ | trifluoromethyl | trifluoromethoxy | Y¹ is CH, Y² is |
| | is chloro | | | CH, Y³ is CH |
| P.0252 | X¹ is chloro, X² is C—Cl, X³ | trifluoromethyl | trifluoromethyl | Y¹ is CH, Y² is |
| | is chloro | | | CH, Y³ is CH |
| P.0253 | X¹ is chloro, X² is C—I, X³ is | chlorodifluoromethyl | bromo | Y¹ is CH, Y² is |
| | chloro | | | CH, Y³ is CH |
| P.0254 | X¹ is chloro, X² is C—I, X³ is | chlorodifluoromethyl | chloro | Y¹ is CH, Y² is |
| | chloro | | | CH, Y³ is CH |
| P.0255 | X¹ is chloro, X² is C—I, X³ is | chlorodifluoromethyl | cyano | Y¹ is CH, Y² is |
| | chloro | | | CH, Y³ is CH |
| P.0256 | X¹ is chloro, X² is C—I, X³ is | chlorodifluoromethyl | cyclopropyl | Y¹ is CH, Y² is |
| | chloro | | | CH, Y³ is CH |
| P.0257 | X¹ is chloro, X² is C—I, X³ is | chlorodifluoromethyl | ethyl | Y¹ is CH, Y² is |
| | chloro | | | CH, Y³ is CH |
| P.0258 | X¹ is chloro, X² is C—I, X³ is | chlorodifluoromethyl | fluoro | Y¹ is CH, Y² is |
| | chloro | | | CH, Y³ is CH |
| P.0259 | X¹ is chloro, X² is C—I, X³ is | chlorodifluoromethyl | hydrogen | Y¹ is CH, Y² is |
| | chloro | | | CH, Y³ is CH |
| P.0260 | X¹ is chloro, X² is C—I, X³ is | chlorodifluoromethyl | methoxy | Y¹ is CH, Y² is |
| | chloro | | | CH, Y³ is CH |
| P.0261 | X¹ is chloro, X² is C—I, X³ is | chlorodifluoromethyl | methyl | Y¹ is CH, Y² is |
| | chloro | | | CH, Y³ is CH |
| P.0262 | X¹ is chloro, X² is C—I, X³ is | chlorodifluoromethyl | nitro | Y¹ is CH, Y² is |
| | chloro | | | CH, Y³ is CH |
| P.0263 | X¹ is chloro, X² is C—I, X³ is | chlorodifluoromethyl | trifluoromethoxy | Y¹ is CH, Y² is |
| | chloro | | | CH, Y³ is CH |
| P.0264 | X¹ is chloro, X² is C—I, X³ is | chlorodifluoromethyl | trifluoromethyl | Y¹ is CH, Y² is |
| | chloro | | | CH, Y³ is CH |
| P.0265 | X¹ is chloro, X² is C—I, X³ is | difluoromethyl | bromo | Y¹ is CH, Y² is |
| | chloro | | | CH, Y³ is CH |
| P.0266 | X¹ is chloro, X² is C—I, X³ is | difluoromethyl | chloro | Y¹ is CH, Y² is |
| | chloro | | | CH, Y³ is CH |
| P.0267 | X¹ is chloro, X² is C—I, X³ is | difluoromethyl | cyano | Y¹ is CH, Y² is |
| | chloro | | | CH, Y³ is CH |
| P.0268 | X¹ is chloro, X² is C—I, X³ is | difluoromethyl | cyclopropyl | Y¹ is CH, Y² is |
| | chloro | | | CH, Y³ is CH |
| P.0269 | X¹ is chloro, X² is C—I, X³ is | difluoromethyl | ethyl | Y¹ is CH, Y² is |
| | chloro | | | CH, Y³ is CH |
| P.0270 | X¹ is chloro, X² is C—I, X³ is | difluoromethyl | fluoro | Y¹ is CH, Y² is |
| | chloro | | | CH, Y³ is CH |
| P.0271 | X¹ is chloro, X² is C—I, X³ is | difluoromethyl | hydrogen | Y¹ is CH, Y² is |
| | chloro | | | CH, Y³ is CH |
| P.0272 | X¹ is chloro, X² is C—I, X³ is | difluoromethyl | methoxy | Y¹ is CH, Y² is |
| | chloro | | | CH, Y³ is CH |
| P.0273 | X¹ is chloro, X² is C—I, X³ is | difluoromethyl | methyl | Y¹ is CH, Y² is |
| | chloro | | | CH, Y³ is CH |
| P.0274 | X¹ is chloro, X² is C—I, X³ is | difluoromethyl | nitro | Y¹ is CH, Y² is |
| | chloro | | | CH, Y³ is CH |
| P.0275 | X¹ is chloro, X² is C—I, X³ is | difluoromethyl | trifluoromethoxy | Y¹ is CH, Y² is |
| | chloro | | | CH, Y³ is CH |
| P.0276 | X¹ is chloro, X² is C—I, X³ is | difluoromethyl | trifluoromethyl | Y¹ is CH, Y² is |
| | chloro | | | CH, Y³ is CH |
| P.0277 | X¹ is chloro, X² is C—I, X³ is | trifluoromethyl | bromo | Y¹ is CH, Y² is |
| | chloro | | | CH, Y³ is CH |
| P.0278 | X¹ is chloro, X² is C—I, X³ is | trifluoromethyl | chloro | Y¹ is CH, Y² is |
| | chloro | | | CH, Y³ is CH |
| P.0279 | X¹ is chloro, X² is C—I, X³ is | trifluoromethyl | cyano | Y¹ is CH, Y² is |
| | chloro | | | CH, Y³ is CH |
| P.0280 | X¹ is chloro, X² is C—I, X³ is | trifluoromethyl | cyclopropyl | Y¹ is CH, Y² is |
| | chloro | | | CH, Y³ is CH |
| P.0281 | X¹ is chloro, X² is C—I, X³ is | trifluoromethyl | ethyl | Y¹ is CH, Y² is |
| | chloro | | | CH, Y³ is CH |
| P.0282 | X¹ is chloro, X² is C—I, X³ is | trifluoromethyl | fluoro | Y¹ is CH, Y² is |
| | chloro | | | CH, Y³ is CH |
| P.0283 | X¹ is chloro, X² is C—I, X³ is | trifluoromethyl | hydrogen | Y¹ is CH, Y² is |
| | chloro | | | CH, Y³ is CH |
| P.0284 | X¹ is chloro, X² is C—I, X³ is | trifluoromethyl | methoxy | Y¹ is CH, Y² is |
| | chloro | | | CH, Y³ is CH |
| P.0285 | X¹ is chloro, X² is C—I, X³ is | trifluoromethyl | methyl | Y¹ is CH, Y² is |
| | chloro | | | CH, Y³ is CH |
| P.0286 | X¹ is chloro, X² is C—I, X³ is | trifluoromethyl | nitro | Y¹ is CH, Y² is |
| | chloro | | | CH, Y³ is CH |
| P.0287 | X¹ is chloro, X² is C—I, X³ is | trifluoromethyl | trifluoromethoxy | Y¹ is CH, Y² is |
| | chloro | | | CH, Y³ is CH |
| P.0288 | X¹ is chloro, X² is C—I, X³ is | trifluoromethyl | trifluoromethyl | Y¹ is CH, Y² is |
| | chloro | | | CH, Y³ is CH |
| P.0289 | X¹ is fluoro, X² is C—F, X³ is | chlorodifluoromethyl | bromo | Y¹ is CH, Y² is |
| | fluoro | | | CH, Y³ is CH |
| P.0290 | X¹ is fluoro, X² is C—F, X³ is | chlorodifluoromethyl | chloro | Y¹ is CH, Y² is |
| | fluoro | | | CH, Y³ is CH |
| P.0291 | X¹ is fluoro, X² is C—F, X³ is | chlorodifluoromethyl | cyano | Y¹ is CH, Y² is |
| | fluoro | | | CH, Y³ is CH |
| P.0292 | X¹ is fluoro, X² is C—F, X³ is | chlorodifluoromethyl | cyclopropyl | Y¹ is CH, Y² is |
| | fluoro | | | CH, Y³ is CH |
| P.0293 | X¹ is fluoro, X² is C—F, X³ is | chlorodifluoromethyl | ethyl | Y¹ is CH, Y² is |
| | fluoro | | | CH, Y³ is CH |
| P.0294 | X¹ is fluoro, X² is C—F, X³ is | chlorodifluoromethyl | fluoro | Y¹ is CH, Y² is |
| | fluoro | | | CH, Y³ is CH |
| P.0295 | X¹ is fluoro, X² is C—F, X³ is | chlorodifluoromethyl | hydrogen | Y¹ is CH, Y² is |
| | fluoro | | | CH, Y³ is CH |
| P.0296 | X¹ is fluoro, X² is C—F, X³ is | chlorodifluoromethyl | methoxy | Y¹ is CH, Y² is |
| | fluoro | | | CH, Y³ is CH |
| P.0297 | X¹ is fluoro, X² is C—F, X³ is | chlorodifluoromethyl | methyl | Y¹ is CH, Y² is |
| | fluoro | | | CH, Y³ is CH |
| P.0298 | X¹ is fluoro, X² is C—F, X³ is | chlorodifluoromethyl | nitro | Y¹ is CH, Y² is |
| | fluoro | | | CH, Y³ is CH |
| P.0299 | X¹ is fluoro, X² is C—F, X³ is | chlorodifluoromethyl | trifluoromethoxy | Y¹ is CH, Y² is |
| | fluoro | | | CH, Y³ is CH |
| P.0300 | X¹ is fluoro, X² is C—F, X³ is | chlorodifluoromethyl | trifluoromethyl | Y¹ is CH, Y² is |
| | fluoro | | | CH, Y³ is CH |
| P.0301 | X¹ is fluoro, X² is C—F, X³ is | difluoromethyl | bromo | Y¹ is CH, Y² is |
| | fluoro | | | CH, Y³ is CH |
| P.0302 | X¹ is fluoro, X² is C—F, X³ is | difluoromethyl | chloro | Y¹ is CH, Y² is |
| | fluoro | | | CH, Y³ is CH |
| P.0303 | X¹ is fluoro, X² is C—F, X³ is | difluoromethyl | cyano | Y¹ is CH, Y² is |
| | fluoro | | | CH, Y³ is CH |
| P.0304 | X¹ is fluoro, X² is C—F, X³ is | difluoromethyl | cyclopropyl | Y¹ is CH, Y² is |
| | fluoro | | | CH, Y³ is CH |
| P.0305 | X¹ is fluoro, X² is C—F, X³ is | difluoromethyl | ethyl | Y¹ is CH, Y² is |
| | fluoro | | | CH, Y³ is CH |
| P.0306 | X¹ is fluoro, X² is C—F, X³ is | difluoromethyl | fluoro | Y¹ is CH, Y² is |
| | fluoro | | | CH, Y³ is CH |
| P.0307 | X¹ is fluoro, X² is C—F, X³ is | difluoromethyl | hydrogen | Y¹ is CH, Y² is |
| | fluoro | | | CH, Y³ is CH |
| P.0308 | X¹ is fluoro, X² is C—F, X³ is | difluoromethyl | methoxy | Y¹ is CH, Y² is |
| | fluoro | | | CH, Y³ is CH |
| P.0309 | X¹ is fluoro, X² is C—F, X³ is | difluoromethyl | methyl | Y¹ is CH, Y² is |
| | fluoro | | | CH, Y³ is CH |
| P.0310 | X¹ is fluoro, X² is C—F, X³ is | difluoromethyl | nitro | Y¹ is CH, Y² is |
| | fluoro | | | CH, Y³ is CH |
| P.0311 | X¹ is fluoro, X² is C—F, X³ is | difluoromethyl | trifluoromethoxy | Y¹ is CH, Y² is |
| | fluoro | | | CH, Y³ is CH |
| P.0312 | X¹ is fluoro, X² is C—F, X³ is | difluoromethyl | trifluoromethyl | Y¹ is CH, Y² is |
| | fluoro | | | CH, Y³ is CH |
| P.0313 | X¹ is fluoro, X² is C—F, X³ is | trifluoromethyl | bromo | Y¹ is CH, Y² is |
| | fluoro | | | CH, Y³ is CH |
| P.0314 | X¹ is fluoro, X² is C—F, X³ is | trifluoromethyl | chloro | Y¹ is CH, Y² is |
| | fluoro | | | CH, Y³ is CH |
| P.0315 | X¹ is fluoro, X² is C—F, X³ is | trifluoromethyl | cyano | Y¹ is CH, Y² is |
| | fluoro | | | CH, Y³ is CH |
| P.0316 | X¹ is fluoro, X² is C—F, X³ is | trifluoromethyl | cyclopropyl | Y¹ is CH, Y² is |
| | fluoro | | | CH, Y³ is CH |
| P.0317 | X¹ is fluoro, X² is C—F, X³ is | trifluoromethyl | ethyl | Y¹ is CH, Y² is |
| | fluoro | | | CH, Y³ is CH |
| P.0318 | X¹ is fluoro, X² is C—F, X³ is | trifluoromethyl | fluoro | Y¹ is CH, Y² is |
| | fluoro | | | CH, Y³ is CH |
| P.0319 | X¹ is fluoro, X² is C—F, X³ is | trifluoromethyl | hydrogen | Y¹ is CH, Y² is |
| | fluoro | | | CH, Y³ is CH |
| P.0320 | X¹ is fluoro, X² is C—F, X³ is | trifluoromethyl | methoxy | Y¹ is CH, Y² is |
| | fluoro | | | CH, Y³ is CH |
| P.0321 | X¹ is fluoro, X² is C—F, X³ is | trifluoromethyl | methyl | Y¹ is CH, Y² is |
| | fluoro | | | CH, Y³ is CH |
| P.0322 | X¹ is fluoro, X² is C—F, X³ is | trifluoromethyl | nitro | Y¹ is CH, Y² is |
| | fluoro | | | CH, Y³ is CH |
| P.0323 | X¹ is fluoro, X² is C—F, X³ is | trifluoromethyl | trifluoromethoxy | Y¹ is CH, Y² is |
| | fluoro | | | CH, Y³ is CH |
| P.0324 | X¹ is fluoro, X² is C—F, X³ is | trifluoromethyl | trifluoromethyl | Y¹ is CH, Y² is |
| | fluoro | | | CH, Y³ is CH |
| P.0325 | X¹ is chloro, X² is CH, X³ is | chlorodifluoromethyl | bromo | Y¹ is CH, Y² is |
| | bromo | | | CH, Y³ is CH |
| P.0326 | X¹ is chloro, X² is CH, X³ is | chlorodifluoromethyl | chloro | Y¹ is CH, Y² is |
| | bromo | | | CH, Y³ is CH |
| P.0327 | X¹ is chloro, X² is CH, X³ is | chlorodifluoromethyl | cyano | Y¹ is CH, Y² is |
| | bromo | | | CH, Y³ is CH |
| P.0328 | X¹ is chloro, X² is CH, X³ is | chlorodifluoromethyl | cyclopropyl | Y¹ is CH, Y² is |
| | bromo | | | CH, Y³ is CH |
| P.0329 | X¹ is chloro, X² is CH, X³ is | chlorodifluoromethyl | ethyl | Y¹ is CH, Y² is |
| | bromo | | | CH, Y³ is CH |
| P.0330 | X¹ is chloro, X² is CH, X³ is | chlorodifluoromethyl | fluoro | Y¹ is CH, Y² is |
| | bromo | | | CH, Y³ is CH |
| P.0331 | X¹ is chloro, X² is CH, X³ is | chlorodifluoromethyl | hydrogen | Y¹ is CH, Y² is |
| | bromo | | | CH, Y³ is CH |
| P.0332 | X¹ is chloro, X² is CH, X³ is | chlorodifluoromethyl | methoxy | Y¹ is CH, Y² is |
| | bromo | | | CH, Y³ is CH |
| P.0333 | X¹ is chloro, X² is CH, X³ is | chlorodifluoromethyl | methyl | Y¹ is CH, Y² is |
| | bromo | | | CH, Y³ is CH |
| P.0334 | X¹ is chloro, X² is CH, X³ is | chlorodifluoromethyl | nitro | Y¹ is CH, Y² is |
| | bromo | | | CH, Y³ is CH |
| P.0335 | X¹ is chloro, X² is CH, X³ is | chlorodifluoromethyl | trifluoromethoxy | Y¹ is CH, Y² is |
| | bromo | | | CH, Y³ is CH |
| P.0336 | X¹ is chloro, X² is CH, X³ is | chlorodifluoromethyl | trifluoromethyl | Y¹ is CH, Y² is |
| | bromo | | | CH, Y³ is CH |
| P.0337 | X¹ is chloro, X² is CH, X³ is | difluoromethyl | bromo | Y¹ is CH, Y² is |
| | bromo | | | CH, Y³ is CH |
| P.0338 | X¹ is chloro, X² is CH, X³ is | difluoromethyl | chloro | Y¹ is CH, Y² is |
| | bromo | | | CH, Y³ is CH |
| P.0339 | X¹ is chloro, X² is CH, X³ is | difluoromethyl | cyano | Y¹ is CH, Y² is |
| | bromo | | | CH, Y³ is CH |
| P.0340 | X¹ is chloro, X² is CH, X³ is | difluoromethyl | cyclopropyl | Y¹ is CH, Y² is |
| | bromo | | | CH, Y³ is CH |
| P.0341 | X¹ is chloro, X² is CH, X³ is | difluoromethyl | ethyl | Y¹ is CH, Y² is |
| | bromo | | | CH, Y³ is CH |
| P.0342 | X¹ is chloro, X² is CH, X³ is | difluoromethyl | fluoro | Y¹ is CH, Y² is |
| | bromo | | | CH, Y³ is CH |
| P.0343 | X¹ is chloro, X² is CH, X³ is | difluoromethyl | hydrogen | Y¹ is CH, Y² is |
| | bromo | | | CH, Y³ is CH |
| P.0344 | X¹ is chloro, X² is CH, X³ is | difluoromethyl | methoxy | Y¹ is CH, Y² is |
| | bromo | | | CH, Y³ is CH |
| P.0345 | X¹ is chloro, X² is CH, X³ is | difluoromethyl | methyl | Y¹ is CH, Y² is |
| | bromo | | | CH, Y³ is CH |
| P.0346 | X¹ is chloro, X² is CH, X³ is | difluoromethyl | nitro | Y¹ is CH, Y² is |
| | bromo | | | CH, Y³ is CH |
| P.0347 | X¹ is chloro, X² is CH, X³ is | difluoromethyl | trifluoromethoxy | Y¹ is CH, Y² is |
| | bromo | | | CH, Y³ is CH |
| P.0348 | X¹ is chloro, X² is CH, X³ is | difluoromethyl | trifluoromethyl | Y¹ is CH, Y² is |
| | bromo | | | CH, Y³ is CH |
| P.0349 | X¹ is chloro, X² is CH, X³ is | trifluoromethyl | bromo | Y¹ is CH, Y² is |
| | bromo | | | CH, Y³ is CH |
| P.0350 | X¹ is chloro, X² is CH, X³ is | trifluoromethyl | chloro | Y¹ is CH, Y² is |
| | bromo | | | CH, Y³ is CH |
| P.0351 | X¹ is chloro, X² is CH, X³ is | trifluoromethyl | cyano | Y¹ is CH, Y² is |
| | bromo | | | CH, Y³ is CH |
| P.0352 | X¹ is chloro, X² is CH, X³ is | trifluoromethyl | cyclopropyl | Y¹ is CH, Y² is |
| | bromo | | | CH, Y³ is CH |
| P.0353 | X¹ is chloro, X² is CH, X³ is | trifluoromethyl | ethyl | Y¹ is CH, Y² is |
| | bromo | | | CH, Y³ is CH |
| P.0354 | X¹ is chloro, X² is CH, X³ is | trifluoromethyl | fluoro | Y¹ is CH, Y² is |
| | bromo | | | CH, Y³ is CH |
| P.0355 | X¹ is chloro, X² is CH, X³ is | trifluoromethyl | hydrogen | Y¹ is CH, Y² is |
| | bromo | | | CH, Y³ is CH |
| P.0356 | X¹ is chloro, X² is CH, X³ is | trifluoromethyl | methoxy | Y¹ is CH, Y² is |
| | bromo | | | CH, Y³ is CH |
| P.0357 | X¹ is chloro, X² is CH, X³ is | trifluoromethyl | methyl | Y¹ is CH, Y² is |
| | bromo | | | CH, Y³ is CH |
| P.0358 | X¹ is chloro, X² is CH, X³ is | trifluoromethyl | nitro | Y¹ is CH, Y² is |
| | bromo | | | CH, Y³ is CH |
| P.0359 | X¹ is chloro, X² is CH, X³ is | trifluoromethyl | trifluoromethoxy | Y¹ is CH, Y² is |
| | bromo | | | CH, Y³ is CH |
| P.0360 | X¹ is chloro, X² is CH, X³ is | trifluoromethyl | trifluoromethyl | Y¹ is CH, Y² is |
| | bromo | | | CH, Y³ is CH |
| P.0361 | X¹ is chloro, X² is CH, X³ is | chlorodifluoromethyl | bromo | Y¹ is CH, Y² is |
| | bromo | | | CH, Y³ is CH |
| P.0362 | X¹ is chloro, X² is CH, X³ is | chlorodifluoromethyl | chloro | Y¹ is CH, Y² is |
| | bromo | | | CH, Y³ is CH |
| P.0363 | X¹ is chloro, X² is CH, X³ is | chlorodifluoromethyl | cyano | Y¹ is CH, Y² is |
| | bromo | | | CH, Y³ is CH |
| P.0364 | X¹ is chloro, X² is CH, X³ is | chlorodifluoromethyl | cyclopropyl | Y¹ is CH, Y² is |
| | bromo | | | CH, Y³ is CH |
| P.0365 | X¹ is chloro, X² is CH, X³ is | chlorodifluoromethyl | ethyl | Y¹ is CH, Y² is |
| | bromo | | | CH, Y³ is CH |
| P.0366 | X¹ is chloro, X² is CH, X³ is | chlorodifluoromethyl | fluoro | Y¹ is CH, Y² is |
| | bromo | | | CH, Y³ is CH |
| P.0367 | X¹ is chloro, X² is CH, X³ is | chlorodifluoromethyl | hydrogen | Y¹ is CH, Y² is |
| | bromo | | | CH, Y³ is CH |
| P.0368 | X¹ is chloro, X² is CH, X³ is | chlorodifluoromethyl | methoxy | Y¹ is CH, Y² is |
| | bromo | | | CH, Y³ is CH |
| P.0369 | X¹ is chloro, X² is CH, X³ is | chlorodifluoromethyl | methyl | Y¹ is CH, Y² is |
| | bromo | | | CH, Y³ is CH |
| P.0370 | X¹ is chloro, X² is CH, X³ is | chlorodifluoromethyl | nitro | Y¹ is CH, Y² is |
| | bromo | | | CH, Y³ is CH |
| P.0371 | X¹ is chloro, X² is CH, X³ is | chlorodifluoromethyl | trifluoromethoxy | Y¹ is CH, Y² is |
| | bromo | | | CH, Y³ is CH |
| P.0372 | X¹ is chloro, X² is CH, X³ is | chlorodifluoromethyl | trifluoromethyl | Y¹ is CH, Y² is |
| | bromo | | | CH, Y³ is CH |
| P.0373 | X¹ is chloro, X² is CH, X³ is | difluoromethyl | bromo | Y¹ is CH, Y² is |
| | bromo | | | CH, Y³ is CH |
| P.0374 | X¹ is chloro, X² is CH, X³ is | difluoromethyl | chloro | Y¹ is CH, Y² is |
| | bromo | | | CH, Y³ is CH |
| P.0375 | X¹ is chloro, X² is CH, X³ is | difluoromethyl | cyano | Y¹ is CH, Y² is |
| | bromo | | | CH, Y³ is CH |
| P.0376 | X¹ is chloro, X² is CH, X³ is | difluoromethyl | cyclopropyl | Y¹ is CH, Y² is |
| | bromo | | | CH, Y³ is CH |
| P.0377 | X¹ is chloro, X² is CH, X³ is | difluoromethyl | ethyl | Y¹ is CH, Y² is |
| | bromo | | | CH, Y³ is CH |
| P.0378 | X¹ is chloro, X² is CH, X³ is | difluoromethyl | fluoro | Y¹ is CH, Y² is |
| | bromo | | | CH, Y³ is CH |
| P.0379 | X¹ is chloro, X² is CH, X³ is | difluoromethyl | hydrogen | Y¹ is CH, Y² is |
| | bromo | | | CH, Y³ is CH |
| P.0380 | X¹ is chloro, X² is CH, X³ is | difluoromethyl | methoxy | Y¹ is CH, Y² is |
| | bromo | | | CH, Y³ is CH |
| P.0381 | X¹ is chloro, X² is CH, X³ is | difluoromethyl | methyl | Y¹ is CH, Y² is |
| | bromo | | | CH, Y³ is CH |
| P.0382 | X¹ is chloro, X² is CH, X³ is | difluoromethyl | nitro | Y¹ is CH, Y² is |
| | bromo | | | CH, Y³ is CH |
| P.0383 | X¹ is chloro, X² is CH, X³ is | difluoromethyl | trifluoromethoxy | Y¹ is CH, Y² is |
| | bromo | | | CH, Y³ is CH |
| P.0384 | X¹ is chloro, X² is CH, X³ is | difluoromethyl | trifluoromethyl | Y¹ is CH, Y² is |
| | bromo | | | CH, Y³ is CH |
| P.0385 | X¹ is chloro, X² is CH, X³ is | trifluoromethyl | bromo | Y¹ is CH, Y² is |
| | bromo | | | CH, Y³ is CH |
| P.0386 | X¹ is chloro, X² is CH, X³ is | trifluoromethyl | chloro | Y¹ is CH, Y² is |
| | bromo | | | CH, Y³ is CH |
| P.0387 | X¹ is chloro, X² is CH, X³ is | trifluoromethyl | cyano | Y¹ is CH, Y² is |
| | bromo | | | CH, Y³ is CH |
| P.0388 | X¹ is chloro, X² is CH, X³ is | trifluoromethyl | cyclopropyl | Y¹ is CH, Y² is |
| | bromo | | | CH, Y³ is CH |
| P.0389 | X¹ is chloro, X² is CH, X³ is | trifluoromethyl | ethyl | Y¹ is CH, Y² is |
| | bromo | | | CH, Y³ is CH |
| P.0390 | X¹ is chloro, X² is CH, X³ is | trifluoromethyl | fluoro | Y¹ is CH, Y² is |
| | bromo | | | CH, Y³ is CH |
| P.0391 | X¹ is chloro, X² is CH, X³ is | trifluoromethyl | hydrogen | Y¹ is CH, Y² is |
| | bromo | | | CH, Y³ is CH |
| P.0392 | X¹ is chloro, X² is CH, X³ is | trifluoromethyl | methoxy | Y¹ is CH, Y² is |
| | bromo | | | CH, Y³ is CH |
| P.0393 | X¹ is chloro, X² is CH, X³ is | trifluoromethyl | methyl | Y¹ is CH, Y² is |
| | bromo | | | CH, Y³ is CH |
| P.0394 | X¹ is chloro, X² is CH, X³ is | trifluoromethyl | nitro | Y¹ is CH, Y² is |
| | bromo | | | CH, Y³ is CH |
| P.0395 | X¹ is chloro, X² is CH, X³ is | trifluoromethyl | trifluoromethoxy | Y¹ is CH, Y² is |
| | bromo | | | CH, Y³ is CH |
| P.0396 | X¹ is chloro, X² is CH, X³ is | trifluoromethyl | trifluoromethyl | Y¹ is CH, Y² is |
| | bromo | | | CH, Y³ is CH |
| P.0397 | X¹ is chloro, X² is CH, X³ is | chlorodifluoromethyl | bromo | Y¹ is CH, Y² is |
| | bromo | | | CH, Y³ is CH |
| P.0398 | X¹ is chloro, X² is CH, X³ is | chlorodifluoromethyl | chloro | Y¹ is CH, Y² is |
| | bromo | | | CH, Y³ is CH |
| P.0399 | X¹ is chloro, X² is CH, X³ is | chlorodifluoromethyl | cyano | Y¹ is CH, Y² is |
| | bromo | | | CH, Y³ is CH |
| P.0400 | X¹ is chloro, X² is CH, X³ is | chlorodifluoromethyl | cyclopropyl | Y¹ is CH, Y² is |
| | bromo | | | CH, Y³ is CH |
| P.0401 | X¹ is chloro, X² is CH, X³ is | chlorodifluoromethyl | ethyl | Y¹ is CH, Y² is |
| | bromo | | | CH, Y³ is CH |
| P.0402 | X¹ is chloro, X² is CH, X³ is | chlorodifluoromethyl | fluoro | Y¹ is CH, Y² is |
| | bromo | | | CH, Y³ is CH |
| P.0403 | X¹ is chloro, X² is CH, X³ is | chlorodifluoromethyl | hydrogen | Y¹ is CH, Y² is |
| | bromo | | | CH, Y³ is CH |
| P.0404 | X¹ is chloro, X² is CH, X³ is | chlorodifluoromethyl | methoxy | Y¹ is CH, Y² is |
| | bromo | | | CH, Y³ is CH |
| P.0405 | X¹ is chloro, X² is CH, X³ is | chlorodifluoromethyl | methyl | Y¹ is CH, Y² is |
| | bromo | | | CH, Y³ is CH |
| P.0406 | X¹ is chloro, X² is CH, X³ is | chlorodifluoromethyl | nitro | Y¹ is CH, Y² is |
| | bromo | | | CH, Y³ is CH |
| P.0407 | X¹ is chloro, X² is CH, X³ is | chlorodifluoromethyl | trifluoromethoxy | Y¹ is CH, Y² is |
| | bromo | | | CH, Y³ is CH |
| P.0408 | X¹ is chloro, X² is CH, X³ is | chlorodifluoromethyl | trifluoromethyl | Y¹ is CH, Y² is |
| | bromo | | | CH, Y³ is CH |
| P.0409 | X¹ is chloro, X² is CH, X³ is | difluoromethyl | bromo | Y¹ is CH, Y² is |
| | bromo | | | CH, Y³ is CH |
| P.0410 | X¹ is chloro, X² is CH, X³ is | difluoromethyl | chloro | Y¹ is CH, Y² is |
| | bromo | | | CH, Y³ is CH |
| P.0411 | X¹ is chloro, X² is CH, X³ is | difluoromethyl | cyano | Y¹ is CH, Y² is |
| | bromo | | | CH, Y³ is CH |
| P.0412 | X¹ is chloro, X² is CH, X³ is | difluoromethyl | cyclopropyl | Y¹ is CH, Y² is |
| | bromo | | | CH, Y³ is CH |
| P.0413 | X¹ is chloro, X² is CH, X³ is | difluoromethyl | ethyl | Y¹ is CH, Y² is |
| | bromo | | | CH, Y³ is CH |
| P.0414 | X¹ is chloro, X² is CH, X³ is | difluoromethyl | fluoro | Y¹ is CH, Y² is |
| | bromo | | | CH, Y³ is CH |
| P.0415 | X¹ is chloro, X² is CH, X³ is | difluoromethyl | hydrogen | Y¹ is CH, Y² is |
| | bromo | | | CH, Y³ is CH |
| P.0416 | X¹ is chloro, X² is CH, X³ is | difluoromethyl | methoxy | Y¹ is CH, Y² is |
| | bromo | | | CH, Y³ is CH |
| P.0417 | X¹ is chloro, X² is CH, X³ is | difluoromethyl | methyl | Y¹ is CH, Y² is |
| | bromo | | | CH, Y³ is CH |
| P.0418 | X¹ is chloro, X² is CH, X³ is | difluoromethyl | nitro | Y¹ is CH, Y² is |
| | bromo | | | CH, Y³ is CH |
| P.0419 | X¹ is chloro, X² is CH, X³ is | difluoromethyl | trifluoromethoxy | Y¹ is CH, Y² is |
| | bromo | | | CH, Y³ is CH |
| P.0420 | X¹ is chloro, X² is CH, X³ is | difluoromethyl | trifluoromethyl | Y¹ is CH, Y² is |
| | bromo | | | CH, Y³ is CH |
| P.0421 | X¹ is chloro, X² is CH, X³ is | trifluoromethyl | bromo | Y¹ is CH, Y² is |
| | bromo | | | CH, Y³ is CH |
| P.0422 | X¹ is chloro, X² is CH, X³ is | trifluoromethyl | chloro | Y¹ is CH, Y² is |
| | bromo | | | CH, Y³ is CH |
| P.0423 | X¹ is chloro, X² is CH, X³ is | trifluoromethyl | cyano | Y¹ is CH, Y² is |
| | bromo | | | CH, Y³ is CH |
| P.0424 | X¹ is chloro, X² is CH, X³ is | trifluoromethyl | cyclopropyl | Y¹ is CH, Y² is |
| | bromo | | | CH, Y³ is CH |
| P.0425 | X¹ is chloro, X² is CH, X³ is | trifluoromethyl | ethyl | Y¹ is CH, Y² is |
| | bromo | | | CH, Y³ is CH |
| P.0426 | X¹ is chloro, X² is CH, X³ is | trifluoromethyl | fluoro | Y¹ is CH, Y² is |
| | bromo | | | CH, Y³ is CH |
| P.0427 | X¹ is chloro, X² is CH, X³ is | trifluoromethyl | hydrogen | Y¹ is CH, Y² is |
| | bromo | | | CH, Y³ is CH |
| P.0428 | X¹ is chloro, X² is CH, X³ is | trifluoromethyl | methoxy | Y¹ is CH, Y² is |
| | bromo | | | CH, Y³ is CH |
| P.0429 | X¹ is chloro, X² is CH, X³ is | trifluoromethyl | methyl | Y¹ is CH, Y² is |
| | bromo | | | CH, Y³ is CH |
| P.0430 | X¹ is chloro, X² is CH, X³ is | trifluoromethyl | nitro | Y¹ is CH, Y² is |
| | bromo | | | CH, Y³ is CH |
| P.0431 | X¹ is chloro, X² is CH, X³ is | trifluoromethyl | trifluoromethoxy | Y¹ is CH, Y² is |
| | bromo | | | CH, Y³ is CH |
| P.0432 | X¹ is chloro, X² is CH, X³ is | trifluoromethyl | trifluoromethyl | Y¹ is CH, Y² is |
| | bromo | | | CH, Y³ is CH |
| P.0433 | X¹ is chloro, X² is C—Cl, X³ | chlorodifluoromethyl | bromo | Y¹ is CH, Y² is |
| | is trifluoromethyl | | | CH, Y³ is CH |
| P.0434 | X¹ is chloro, X² is C—Cl, X³ | chlorodifluoromethyl | chloro | Y¹ is CH, Y² is |
| | is trifluoromethyl | | | CH, Y³ is CH |
| P.0435 | X¹ is chloro, X² is C—Cl, X³ | chlorodifluoromethyl | cyano | Y¹ is CH, Y² is |
| | is trifluoromethyl | | | CH, Y³ is CH |
| P.0436 | X¹ is chloro, X² is C—Cl, X³ | chlorodifluoromethyl | cyclopropyl | Y¹ is CH, Y² is |
| | is trifluoromethyl | | | CH, Y³ is CH |
| P.0437 | X¹ is chloro, X² is C—Cl, X³ | chlorodifluoromethyl | ethyl | Y¹ is CH, Y² is |
| | is trifluoromethyl | | | CH, Y³ is CH |
| P.0438 | X¹ is chloro, X² is C—Cl, X³ | chlorodifluoromethyl | fluoro | Y¹ is CH, Y² is |
| | is trifluoromethyl | | | CH, Y³ is CH |
| P.0439 | X¹ is chloro, X² is C—Cl, X³ | chlorodifluoromethyl | hydrogen | Y¹ is CH, Y² is |
| | is trifluoromethyl | | | CH, Y³ is CH |
| P.0440 | X¹ is chloro, X² is C—Cl, X³ | chlorodifluoromethyl | methoxy | Y¹ is CH, Y² is |
| | is trifluoromethyl | | | CH, Y³ is CH |
| P.0441 | X¹ is chloro, X² is C—Cl, X³ | chlorodifluoromethyl | methyl | Y¹ is CH, Y² is |
| | is trifluoromethyl | | | CH, Y³ is CH |
| P.0442 | X¹ is chloro, X² is C—Cl, X³ | chlorodifluoromethyl | nitro | Y¹ is CH, Y² is |
| | is trifluoromethyl | | | CH, Y³ is CH |
| P.0443 | X¹ is chloro, X² is C—Cl, X³ | chlorodifluoromethyl | trifluoromethoxy | Y¹ is CH, Y² is |
| | is trifluoromethyl | | | CH, Y³ is CH |
| P.0444 | X¹ is chloro, X² is C—Cl, X³ | chlorodifluoromethyl | trifluoromethyl | Y¹ is CH, Y² is |
| | is trifluoromethyl | | | CH, Y³ is CH |
| P.0445 | X¹ is chloro, X² is C—Cl, X³ | difluoromethyl | bromo | Y¹ is CH, Y² is |
| | is trifluoromethyl | | | CH, Y³ is CH |
| P.0446 | X¹ is chloro, X² is C—Cl, X³ | difluoromethyl | chloro | Y¹ is CH, Y² is |
| | is trifluoromethyl | | | CH, Y³ is CH |
| P.0447 | X¹ is chloro, X² is C—Cl, X³ | difluoromethyl | cyano | Y¹ is CH, Y² is |
| | is trifluoromethyl | | | CH, Y³ is CH |
| P.0448 | X¹ is chloro, X² is C—Cl, X³ | difluoromethyl | cyclopropyl | Y¹ is CH, Y² is |
| | is trifluoromethyl | | | CH, Y³ is CH |
| P.0449 | X¹ is chloro, X² is C—Cl, X³ | difluoromethyl | ethyl | Y¹ is CH, Y² is |
| | is trifluoromethyl | | | CH, Y³ is CH |
| P.0450 | X¹ is chloro, X² is C—Cl, X³ | difluoromethyl | fluoro | Y¹ is CH, Y² is |
| | is trifluoromethyl | | | CH, Y³ is CH |
| P.0451 | X¹ is chloro, X² is C—Cl, X³ | difluoromethyl | hydrogen | Y¹ is CH, Y² is |
| | is trifluoromethyl | | | CH, Y³ is CH |
| P.0452 | X¹ is chloro, X² is C—Cl, X³ | difluoromethyl | methoxy | Y¹ is CH, Y² is |
| | is trifluoromethyl | | | CH, Y³ is CH |
| P.0453 | X¹ is chloro, X² is C—Cl, X³ | difluoromethyl | methyl | Y¹ is CH, Y² is |
| | is trifluoromethyl | | | CH, Y³ is CH |
| P.0454 | X¹ is chloro, X² is C—Cl, X³ | difluoromethyl | nitro | Y¹ is CH, Y² is |
| | is trifluoromethyl | | | CH, Y³ is CH |
| P.0455 | X¹ is chloro, X² is C—Cl, X³ | difluoromethyl | trifluoromethoxy | Y¹ is CH, Y² is |
| | is trifluoromethyl | | | CH, Y³ is CH |
| P.0456 | X¹ is chloro, X² is C—Cl, X³ | difluoromethyl | trifluoromethyl | Y¹ is CH, Y² is |
| | is trifluoromethyl | | | CH, Y³ is CH |
| P.0457 | X¹ is chloro, X² is C—Cl, X³ | trifluoromethyl | bromo | Y¹ is CH, Y² is |
| | is trifluoromethyl | | | CH, Y³ is CH |
| P.0458 | X¹ is chloro, X² is C—Cl, X³ | trifluoromethyl | chloro | Y¹ is CH, Y² is |
| | is trifluoromethyl | | | CH, Y³ is CH |
| P.0459 | X¹ is chloro, X² is C—Cl, X³ | trifluoromethyl | cyano | Y¹ is CH, Y² is |
| | is trifluoromethyl | | | CH, Y³ is CH |
| P.0460 | X¹ is chloro, X² is C—Cl, X³ | trifluoromethyl | cyclopropyl | Y¹ is CH, Y² is |
| | is trifluoromethyl | | | CH, Y³ is CH |
| P.0461 | X¹ is chloro, X² is C—Cl, X³ | trifluoromethyl | ethyl | Y¹ is CH, Y² is |
| | is trifluoromethyl | | | CH, Y³ is CH |
| P.0462 | X¹ is chloro, X² is C—Cl, X³ | trifluoromethyl | fluoro | Y¹ is CH, Y² is |
| | is trifluoromethyl | | | CH, Y³ is CH |
| P.0463 | X¹ is chloro, X² is C—Cl, X³ | trifluoromethyl | hydrogen | Y¹ is CH, Y² is |
| | is trifluoromethyl | | | CH, Y³ is CH |
| P.0464 | X¹ is chloro, X² is C—Cl, X³ | trifluoromethyl | methoxy | Y¹ is CH, Y² is |
| | is trifluoromethyl | | | CH, Y³ is CH |
| P.0465 | X¹ is chloro, X² is C—Cl, X³ | trifluoromethyl | methyl | Y¹ is CH, Y² is |
| | is trifluoromethyl | | | CH, Y³ is CH |
| P.0466 | X¹ is chloro, X² is C—Cl, X³ | trifluoromethyl | nitro | Y¹ is CH, Y² is |
| | is trifluoromethyl | | | CH, Y³ is CH |
| P.0467 | X¹ is chloro, X² is C—Cl, X³ | trifluoromethyl | trifluoromethoxy | Y¹ is CH, Y² is |
| | is trifluoromethyl | | | CH, Y³ is CH |
| P.0468 | X¹ is chloro, X² is C—Cl, X³ | trifluoromethyl | trifluoromethyl | Y¹ is CH, Y² is |
| | is trifluoromethyl | | | CH, Y³ is CH |
| P.0469 | X¹ is trifluoromethyl, X² is | chlorodifluoromethyl | bromo | Y¹ is CH, Y² is |
| | CH, X³ is trifluoromethyl | | | CH, Y³ is CH |
| P.0470 | X¹ is trifluoromethyl, X² is | chlorodifluoromethyl | chloro | Y¹ is CH, Y² is |
| | CH, X³ is trifluoromethyl | | | CH, Y³ is CH |
| P.0471 | X¹ is trifluoromethyl, X² is | chlorodifluoromethyl | cyano | Y¹ is CH, Y² is |
| | CH, X³ is trifluoromethyl | | | CH, Y³ is CH |
| P.0472 | X¹ is trifluoromethyl, X² is | chlorodifluoromethyl | cyclopropyl | Y¹ is CH, Y² is |
| | CH, X³ is trifluoromethyl | | | CH, Y³ is CH |
| P.0473 | X¹ is trifluoromethyl, X² is | chlorodifluoromethyl | ethyl | Y¹ is CH, Y² is |
| | CH, X³ is trifluoromethyl | | | CH, Y³ is CH |
| P.0474 | X¹ is trifluoromethyl, X² is | chlorodifluoromethyl | fluoro | Y¹ is CH, Y² is |
| | CH, X³ is trifluoromethyl | | | CH, Y³ is CH |
| P.0475 | X¹ is trifluoromethyl, X² is | chlorodifluoromethyl | hydrogen | Y¹ is CH, Y² is |
| | CH, X³ is trifluoromethyl | | | CH, Y³ is CH |
| P.0476 | X¹ is trifluoromethyl, X² is | chlorodifluoromethyl | methoxy | Y¹ is CH, Y² is |
| | CH, X³ is trifluoromethyl | | | CH, Y³ is CH |
| P.0477 | X¹ is trifluoromethyl, X² is | chlorodifluoromethyl | methyl | Y¹ is CH, Y² is |
| | CH, X³ is trifluoromethyl | | | CH, Y³ is CH |
| P.0478 | X¹ is trifluoromethyl, X² is | chlorodifluoromethyl | nitro | Y¹ is CH, Y² is |
| | CH, X³ is trifluoromethyl | | | CH, Y³ is CH |
| P.0479 | X¹ is trifluoromethyl, X² is | chlorodifluoromethyl | trifluoromethoxy | Y¹ is CH, Y² is |
| | CH, X³ is trifluoromethyl | | | CH, Y³ is CH |
| P.0480 | X¹ is trifluoromethyl, X² is | chlorodifluoromethyl | trifluoromethyl | Y¹ is CH, Y² is |
| | CH, X³ is trifluoromethyl | | | CH, Y³ is CH |
| P.0481 | X¹ is trifluoromethyl, X² is | difluoromethyl | bromo | Y¹ is CH, Y² is |
| | CH, X³ is trifluoromethyl | | | CH, Y³ is CH |
| P.0482 | X¹ is trifluoromethyl, X² is | difluoromethyl | chloro | Y¹ is CH, Y² is |
| | CH, X³ is trifluoromethyl | | | CH, Y³ is CH |
| P.0483 | X¹ is trifluoromethyl, X² is | difluoromethyl | cyano | Y¹ is CH, Y² is |
| | CH, X³ is trifluoromethyl | | | CH, Y³ is CH |
| P.0484 | X¹ is trifluoromethyl, X² is | difluoromethyl | cyclopropyl | Y¹ is CH, Y² is |
| | CH, X³ is trifluoromethyl | | | CH, Y³ is CH |
| P.0485 | X¹ is trifluoromethyl, X² is | difluoromethyl | ethyl | Y¹ is CH, Y² is |
| | CH, X³ is trifluoromethyl | | | CH, Y³ is CH |
| P.0486 | X¹ is trifluoromethyl, X² is | difluoromethyl | fluoro | Y¹ is CH, Y² is |
| | CH, X³ is trifluoromethyl | | | CH, Y³ is CH |
| P.0487 | X¹ is trifluoromethyl, X² is | difluoromethyl | hydrogen | Y¹ is CH, Y² is |
| | CH, X³ is trifluoromethyl | | | CH, Y³ is CH |
| P.0488 | X¹ is trifluoromethyl, X² is | difluoromethyl | methoxy | Y¹ is CH, Y² is |
| | CH, X³ is trifluoromethyl | | | CH, Y³ is CH |
| P.0489 | X¹ is trifluoromethyl, X² is | difluoromethyl | methyl | Y¹ is CH, Y² is |
| | CH, X³ is trifluoromethyl | | | CH, Y³ is CH |
| P.0490 | X¹ is trifluoromethyl, X² is | difluoromethyl | nitro | Y¹ is CH, Y² is |
| | CH, X³ is trifluoromethyl | | | CH, Y³ is CH |
| P.0491 | X¹ is trifluoromethyl, X² is | difluoromethyl | trifluoromethoxy | Y¹ is CH, Y² is |
| | CH, X³ is trifluoromethyl | | | CH, Y³ is CH |
| P.0492 | X¹ is trifluoromethyl, X² is | difluoromethyl | trifluoromethyl | Y¹ is CH, Y² is |
| | CH, X³ is trifluoromethyl | | | CH, Y³ is CH |
| P.0493 | X¹ is trifluoromethyl, X² is | trifluoromethyl | bromo | Y¹ is CH, Y² is |
| | CH, X³ is trifluoromethyl | | | CH, Y³ is CH |
| P.0494 | X¹ is trifluoromethyl, X² is | trifluoromethyl | chloro | Y¹ is CH, Y² is |
| | CH, X³ is trifluoromethyl | | | CH, Y³ is CH |
| P.0495 | X¹ is trifluoromethyl, X² is | trifluoromethyl | cyano | Y¹ is CH, Y² is |
| | CH, X³ is trifluoromethyl | | | CH, Y³ is CH |
| P.0496 | X¹ is trifluoromethyl, X² is | trifluoromethyl | cyclopropyl | Y¹ is CH, Y² is |
| | CH, X³ is trifluoromethyl | | | CH, Y³ is CH |
| P.0497 | X¹ is trifluoromethyl, X² is | trifluoromethyl | ethyl | Y¹ is CH, Y² is |
| | CH, X³ is trifluoromethyl | | | CH, Y³ is CH |
| P.0498 | X¹ is trifluoromethyl, X² is | trifluoromethyl | fluoro | Y¹ is CH, Y² is |
| | CH, X³ is trifluoromethyl | | | CH, Y³ is CH |
| P.0499 | X¹ is trifluoromethyl, X² is | trifluoromethyl | hydrogen | Y¹ is CH, Y² is |
| | CH, X³ is trifluoromethyl | | | CH, Y³ is CH |
| P.0500 | X¹ is trifluoromethyl, X² is | trifluoromethyl | methoxy | Y¹ is CH, Y² is |
| | CH, X³ is trifluoromethyl | | | CH, Y³ is CH |
| P.0501 | X¹ is trifluoromethyl, X² is | trifluoromethyl | methyl | Y¹ is CH, Y² is |
| | CH, X³ is trifluoromethyl | | | CH, Y³ is CH |
| P.0502 | X¹ is trifluoromethyl, X² is | trifluoromethyl | nitro | Y¹ is CH, Y² is |
| | CH, X³ is trifluoromethyl | | | CH, Y³ is CH |
| P.0503 | X¹ is trifluoromethyl, X² is | trifluoromethyl | trifluoromethoxy | Y¹ is CH, Y² is |
| | CH, X³ is trifluoromethyl | | | CH, Y³ is CH |
| P.0504 | X¹ is trifluoromethyl, X² is | trifluoromethyl | trifluoromethyl | Y¹ is CH, Y² is |
| | CH, X³ is trifluoromethyl | | | CH, Y³ is CH |
| P.0505 | X¹ is trifluoromethyl, X² is | chlorodifluoromethyl | bromo | Y¹ is CH, Y² is |
| | CH, X³ is trifluoromethyl | | | CH, Y³ is CH |
| P.0506 | X¹ is trifluoromethyl, X² is | chlorodifluoromethyl | chloro | Y¹ is CH, Y² is |
| | CH, X³ is trifluoromethyl | | | CH, Y³ is CH |
| P.0507 | X¹ is trifluoromethyl, X² is | chlorodifluoromethyl | cyano | Y¹ is CH, Y² is |
| | CH, X³ is trifluoromethyl | | | CH, Y³ is CH |
| P.0508 | X¹ is trifluoromethyl, X² is | chlorodifluoromethyl | cyclopropyl | Y¹ is CH, Y² is |
| | CH, X³ is trifluoromethyl | | | CH, Y³ is CH |
| P.0509 | X¹ is trifluoromethyl, X² is | chlorodifluoromethyl | ethyl | Y¹ is CH, Y² is |
| | CH, X³ is trifluoromethyl | | | CH, Y³ is CH |
| P.0510 | X¹ is trifluoromethyl, X² is | chlorodifluoromethyl | fluoro | Y¹ is CH, Y² is |
| | CH, X³ is trifluoromethyl | | | CH, Y³ is CH |
| P.0511 | X¹ is trifluoromethyl, X² is | chlorodifluoromethyl | hydrogen | Y¹ is CH, Y² is |
| | CH, X³ is trifluoromethyl | | | CH, Y³ is CH |
| P.0512 | X¹ is trifluoromethyl, X² is | chlorodifluoromethyl | methoxy | Y¹ is CH, Y² is |
| | CH, X³ is trifluoromethyl | | | CH, Y³ is CH |
| P.0513 | X¹ is trifluoromethyl, X² is | chlorodifluoromethyl | methyl | Y¹ is CH, Y² is |
| | CH, X³ is trifluoromethyl | | | CH, Y³ is CH |
| P.0514 | X¹ is trifluoromethyl, X² is | chlorodifluoromethyl | nitro | Y¹ is CH, Y² is |
| | CH, X³ is trifluoromethyl | | | CH, Y³ is CH |
| P.0515 | X¹ is trifluoromethyl, X² is | chlorodifluoromethyl | trifluoromethoxy | Y¹ is CH, Y² is |
| | CH, X³ is trifluoromethyl | | | CH, Y³ is CH |
| P.0516 | X¹ is trifluoromethyl, X² is | chlorodifluoromethyl | trifluoromethyl | Y¹ is CH, Y² is |
| | CH, X³ is trifluoromethyl | | | CH, Y³ is CH |
| P.0517 | X¹ is trifluoromethyl, X² is | difluoromethyl | bromo | Y¹ is CH, Y² is |
| | CH, X³ is trifluoromethyl | | | CH, Y³ is CH |
| P.0518 | X¹ is trifluoromethyl, X² is | difluoromethyl | chloro | Y¹ is CH, Y² is |
| | CH, X³ is trifluoromethyl | | | CH, Y³ is CH |
| P.0519 | X¹ is trifluoromethyl, X² is | difluoromethyl | cyano | Y¹ is CH, Y² is |
| | CH, X³ is trifluoromethyl | | | CH, Y³ is CH |
| P.0520 | X¹ is trifluoromethyl, X² is | difluoromethyl | cyclopropyl | Y¹ is CH, Y² is |
| | CH, X³ is trifluoromethyl | | | CH, Y³ is CH |
| P.0521 | X¹ is trifluoromethyl, X² is | difluoromethyl | ethyl | Y¹ is CH, Y² is |
| | CH, X³ is trifluoromethyl | | | CH, Y³ is CH |
| P.0522 | X¹ is trifluoromethyl, X² is | difluoromethyl | fluoro | Y¹ is CH, Y² is |
| | CH, X³ is trifluoromethyl | | | CH, Y³ is CH |
| P.0523 | X¹ is trifluoromethyl, X² is | difluoromethyl | hydrogen | Y¹ is CH, Y² is |
| | CH, X³ is trifluoromethyl | | | CH, Y³ is CH |
| P.0524 | X¹ is trifluoromethyl, X² is | difluoromethyl | methoxy | Y¹ is CH, Y² is |
| | CH, X³ is trifluoromethyl | | | CH, Y³ is CH |
| P.0525 | X¹ is trifluoromethyl, X² is | difluoromethyl | methyl | Y¹ is CH, Y² is |
| | CH, X³ is trifluoromethyl | | | CH, Y³ is CH |
| P.0526 | X¹ is trifluoromethyl, X² is | difluoromethyl | nitro | Y¹ is CH, Y² is |
| | CH, X³ is trifluoromethyl | | | CH, Y³ is CH |
| P.0527 | X¹ is trifluoromethyl, X² is | difluoromethyl | trifluoromethoxy | Y¹ is CH, Y² is |
| | CH, X³ is trifluoromethyl | | | CH, Y³ is CH |
| P.0528 | X¹ is trifluoromethyl, X² is | difluoromethyl | trifluoromethyl | Y¹ is CH, Y² is |
| | CH, X³ is trifluoromethyl | | | CH, Y³ is CH |
| P.0529 | X¹ is trifluoromethyl, X² is | trifluoromethyl | bromo | Y¹ is CH, Y² is |
| | CH, X³ is trifluoromethyl | | | CH, Y³ is CH |
| P.0530 | X¹ is trifluoromethyl, X² is | trifluoromethyl | chloro | Y¹ is CH, Y² is |
| | CH, X³ is trifluoromethyl | | | CH, Y³ is CH |
| P.0531 | X¹ is trifluoromethyl, X² is | trifluoromethyl | cyano | Y¹ is CH, Y² is |
| | CH, X³ is trifluoromethyl | | | CH, Y³ is CH |
| P.0532 | X¹ is trifluoromethyl, X² is | trifluoromethyl | cyclopropyl | Y¹ is CH, Y² is |
| | CH, X³ is trifluoromethyl | | | CH, Y³ is CH |
| P.0533 | X¹ is trifluoromethyl, X² is | trifluoromethyl | ethyl | Y¹ is CH, Y² is |
| | CH, X³ is trifluoromethyl | | | CH, Y³ is CH |
| P.0534 | X¹ is trifluoromethyl, X² is | trifluoromethyl | fluoro | Y¹ is CH, Y² is |
| | CH, X³ is trifluoromethyl | | | CH, Y³ is CH |
| P.0535 | X¹ is trifluoromethyl, X² is | trifluoromethyl | hydrogen | Y¹ is CH, Y² is |
| | CH, X³ is trifluoromethyl | | | CH, Y³ is CH |
| P.0536 | X¹ is trifluoromethyl, X² is | trifluoromethyl | methoxy | Y¹ is CH, Y² is |
| | CH, X³ is trifluoromethyl | | | CH, Y³ is CH |
| P.0537 | X¹ is trifluoromethyl, X² is | trifluoromethyl | methyl | Y¹ is CH, Y² is |
| | CH, X³ is trifluoromethyl | | | CH, Y³ is CH |
| P.0538 | X¹ is trifluoromethyl, X² is | trifluoromethyl | nitro | Y¹ is CH, Y² is |
| | CH, X³ is trifluoromethyl | | | CH, Y³ is CH |
| P.0539 | X¹ is trifluoromethyl, X² is | trifluoromethyl | trifluoromethoxy | Y¹ is CH, Y² is |
| | CH, X³ is trifluoromethyl | | | CH, Y³ is CH |
| P.0540 | X¹ is trifluoromethyl, X² is | trifluoromethyl | trifluoromethyl | Y¹ is CH, Y² is |
| | CH, X³ is trifluoromethyl | | | CH, Y³ is CH |
| P.0541 | X¹ is trifluoromethyl, X² is | chlorodifluoromethyl | bromo | Y¹ is CH, Y² is |
| | CH, X³ is hydrogen | | | CH, Y³ is CH |
| P.0542 | X¹ is trifluoromethyl, X² is | chlorodifluoromethyl | chloro | Y¹ is CH, Y² is |
| | CH, X³ is hydrogen | | | CH, Y³ is CH |
| P.0543 | X¹ is trifluoromethyl, X² is | chlorodifluoromethyl | cyano | Y¹ is CH, Y² is |
| | CH, X³ is hydrogen | | | CH, Y³ is CH |
| P.0544 | X¹ is trifluoromethyl, X² is | chlorodifluoromethyl | cyclopropyl | Y¹ is CH, Y² is |
| | CH, X³ is hydrogen | | | CH, Y³ is CH |
| P.0545 | X¹ is trifluoromethyl, X² is | chlorodifluoromethyl | ethyl | Y¹ is CH, Y² is |
| | CH, X³ is hydrogen | | | CH, Y³ is CH |
| P.0546 | X¹ is trifluoromethyl, X² is | chlorodifluoromethyl | fluoro | Y¹ is CH, Y² is |
| | CH, X³ is hydrogen | | | CH, Y³ is CH |
| P.0547 | X¹ is trifluoromethyl, X² is | chlorodifluoromethyl | hydrogen | Y¹ is CH, Y² is |
| | CH, X³ is hydrogen | | | CH, Y³ is CH |
| P.0548 | X¹ is trifluoromethyl, X² is | chlorodifluoromethyl | methoxy | Y¹ is CH, Y² is |
| | CH, X³ is hydrogen | | | CH, Y³ is CH |
| P.0549 | X¹ is trifluoromethyl, X² is | chlorodifluoromethyl | methyl | Y¹ is CH, Y² is |
| | CH, X³ is hydrogen | | | CH, Y³ is CH |
| P.0550 | X¹ is trifluoromethyl, X² is | chlorodifluoromethyl | nitro | Y¹ is CH, Y² is |
| | CH, X³ is hydrogen | | | CH, Y³ is CH |
| P.0551 | X¹ is trifluoromethyl, X² is | chlorodifluoromethyl | trifluoromethoxy | Y¹ is CH, Y² is |
| | CH, X³ is hydrogen | | | CH, Y³ is CH |
| P.0552 | X¹ is trifluoromethyl, X² is | chlorodifluoromethyl | trifluoromethyl | Y¹ is CH, Y² is |
| | CH, X³ is hydrogen | | | CH, Y³ is CH |
| P.0553 | X¹ is trifluoromethyl, X² is | difluoromethyl | bromo | Y¹ is CH, Y² is |
| | CH, X³ is hydrogen | | | CH, Y³ is CH |
| P.0554 | X¹ is trifluoromethyl, X² is | difluoromethyl | chloro | Y¹ is CH, Y² is |
| | CH, X³ is hydrogen | | | CH, Y³ is CH |
| P.0555 | X¹ is trifluoromethyl, X² is | difluoromethyl | cyano | Y¹ is CH, Y² is |
| | CH, X³ is hydrogen | | | CH, Y³ is CH |
| P.0556 | X¹ is trifluoromethyl, X² is | difluoromethyl | cyclopropyl | Y¹ is CH, Y² is |
| | CH, X³ is hydrogen | | | CH, Y³ is CH |
| P.0557 | X¹ is trifluoromethyl, X² is | difluoromethyl | ethyl | Y¹ is CH, Y² is |
| | CH, X³ is hydrogen | | | CH, Y³ is CH |
| P.0558 | X¹ is trifluoromethyl, X² is | difluoromethyl | fluoro | Y¹ is CH, Y² is |
| | CH, X³ is hydrogen | | | CH, Y³ is CH |
| P.0559 | X¹ is trifluoromethyl, X² is | difluoromethyl | hydrogen | Y¹ is CH, Y² is |
| | CH, X³ is hydrogen | | | CH, Y³ is CH |
| P.0560 | X¹ is trifluoromethyl, X² is | difluoromethyl | methoxy | Y¹ is CH, Y² is |
| | CH, X³ is hydrogen | | | CH, Y³ is CH |
| P.0561 | X¹ is trifluoromethyl, X² is | difluoromethyl | methyl | Y¹ is CH, Y² is |
| | CH, X³ is hydrogen | | | CH, Y³ is CH |
| P.0562 | X¹ is trifluoromethyl, X² is | difluoromethyl | nitro | Y¹ is CH, Y² is |
| | CH, X³ is hydrogen | | | CH, Y³ is CH |
| P.0563 | X¹ is trifluoromethyl, X² is | difluoromethyl | trifluoromethoxy | Y¹ is CH, Y² is |
| | CH, X³ is hydrogen | | | CH, Y³ is CH |
| P.0564 | X¹ is trifluoromethyl, X² is | difluoromethyl | trifluoromethyl | Y¹ is CH, Y² is |
| | CH, X³ is hydrogen | | | CH, Y³ is CH |
| P.0565 | X¹ is trifluoromethyl, X² is | trifluoromethyl | bromo | Y¹ is CH, Y² is |
| | CH, X³ is hydrogen | | | CH, Y³ is CH |
| P.0566 | X¹ is trifluoromethyl, X² is | trifluoromethyl | chloro | Y¹ is CH, Y² is |
| | CH, X³ is hydrogen | | | CH, Y³ is CH |
| P.0567 | X¹ is trifluoromethyl, X² is | trifluoromethyl | cyano | Y¹ is CH, Y² is |
| | CH, X³ is hydrogen | | | CH, Y³ is CH |
| P.0568 | X¹ is trifluoromethyl, X² is | trifluoromethyl | cyclopropyl | Y¹ is CH, Y² is |
| | CH, X³ is hydrogen | | | CH, Y³ is CH |
| P.0569 | X¹ is trifluoromethyl, X² is | trifluoromethyl | ethyl | Y¹ is CH, Y² is |
| | CH, X³ is hydrogen | | | CH, Y³ is CH |
| P.0570 | X¹ is trifluoromethyl, X² is | trifluoromethyl | fluoro | Y¹ is CH, Y² is |
| | CH, X³ is hydrogen | | | CH, Y³ is CH |
| P.0571 | X¹ is trifluoromethyl, X² is | trifluoromethyl | hydrogen | Y¹ is CH, Y² is |
| | CH, X³ is hydrogen | | | CH, Y³ is CH |
| P.0572 | X¹ is trifluoromethyl, X² is | trifluoromethyl | methoxy | Y¹ is CH, Y² is |
| | CH, X³ is hydrogen | | | CH, Y³ is CH |
| P.0573 | X¹ is trifluoromethyl, X² is | trifluoromethyl | methyl | Y¹ is CH, Y² is |
| | CH, X³ is hydrogen | | | CH, Y³ is CH |
| P.0574 | X¹ is trifluoromethyl, X² is | trifluoromethyl | nitro | Y¹ is CH, Y² is |
| | CH, X³ is hydrogen | | | CH, Y³ is CH |
| P.0575 | X¹ is trifluoromethyl, X² is | trifluoromethyl | trifluoromethoxy | Y¹ is CH, Y² is |
| | CH, X³ is hydrogen | | | CH, Y³ is CH |
| P.0576 | X¹ is trifluoromethyl, X² is | trifluoromethyl | trifluoromethyl | Y¹ is CH, Y² is |
| | CH, X³ is hydrogen | | | CH, Y³ is CH |
| P.0577 | X¹ is chloro, X² is N, X³ is | chlorodifluoromethyl | bromo | Y¹ is CH, Y² is |
| | chloro | | | CH, Y³ is CH |
| P.0578 | X¹ is chloro, X² is N, X³ is | chlorodifluoromethyl | chloro | Y¹ is CH, Y² is |
| | chloro | | | CH, Y³ is CH |
| P.0579 | X¹ is chloro, X² is N, X³ is | chlorodifluoromethyl | cyano | Y¹ is CH, Y² is |
| | chloro | | | CH, Y³ is CH |
| P.0580 | X¹ is chloro, X² is N, X³ is | chlorodifluoromethyl | cyclopropyl | Y¹ is CH, Y² is |
| | chloro | | | CH, Y³ is CH |
| P.0581 | X¹ is chloro, X² is N, X³ is | chlorodifluoromethyl | ethyl | Y¹ is CH, Y² is |
| | chloro | | | CH, Y³ is CH |
| P.0582 | X¹ is chloro, X² is N, X³ is | chlorodifluoromethyl | fluoro | Y¹ is CH, Y² is |
| | chloro | | | CH, Y³ is CH |
| P.0583 | X¹ is chloro, X² is N, X³ is | chlorodifluoromethyl | hydrogen | Y¹ is CH, Y² is |
| | chloro | | | CH, Y³ is CH |
| P.0584 | X¹ is chloro, X² is N, X³ is | chlorodifluoromethyl | methoxy | Y¹ is CH, Y² is |
| | chloro | | | CH, Y³ is CH |
| P.0585 | X¹ is chloro, X² is N, X³ is | chlorodifluoromethyl | methyl | Y¹ is CH, Y² is |
| | chloro | | | CH, Y³ is CH |
| P.0586 | X¹ is chloro, X² is N, X³ is | chlorodifluoromethyl | nitro | Y¹ is CH, Y² is |
| | chloro | | | CH, Y³ is CH |
| P.0587 | X¹ is chloro, X² is N, X³ is | chlorodifluoromethyl | trifluoromethoxy | Y¹ is CH, Y² is |
| | chloro | | | CH, Y³ is CH |
| P.0588 | X¹ is chloro, X² is N, X³ is | chlorodifluoromethyl | trifluoromethyl | Y¹ is CH, Y² is |
| | chloro | | | CH, Y³ is CH |
| P.0589 | X¹ is chloro, X² is N, X³ is | difluoromethyl | bromo | Y¹ is CH, Y² is |
| | chloro | | | CH, Y³ is CH |
| P.0590 | X¹ is chloro, X² is N, X³ is | difluoromethyl | chloro | Y¹ is CH, Y² is |
| | chloro | | | CH, Y³ is CH |
| P.0591 | X¹ is chloro, X² is N, X³ is | difluoromethyl | cyano | Y¹ is CH, Y² is |
| | chloro | | | CH, Y³ is CH |
| P.0592 | X¹ is chloro, X² is N, X³ is | difluoromethyl | cyclopropyl | Y¹ is CH, Y² is |
| | chloro | | | CH, Y³ is CH |
| P.0593 | X¹ is chloro, X² is N, X³ is | difluoromethyl | ethyl | Y¹ is CH, Y² is |
| | chloro | | | CH, Y³ is CH |
| P.0594 | X¹ is chloro, X² is N, X³ is | difluoromethyl | fluoro | Y¹ is CH, Y² is |
| | chloro | | | CH, Y³ is CH |
| P.0595 | X¹ is chloro, X² is N, X³ is | difluoromethyl | hydrogen | Y¹ is CH, Y² is |
| | chloro | | | CH, Y³ is CH |
| P.0596 | X¹ is chloro, X² is N, X³ is | difluoromethyl | methoxy | Y¹ is CH, Y² is |
| | chloro | | | CH, Y³ is CH |
| P.0597 | X¹ is chloro, X² is N, X³ is | difluoromethyl | methyl | Y¹ is CH, Y² is |
| | chloro | | | CH, Y³ is CH |
| P.0598 | X¹ is chloro, X² is N, X³ is | difluoromethyl | nitro | Y¹ is CH, Y² is |
| | chloro | | | CH, Y³ is CH |
| P.0599 | X¹ is chloro, X² is N, X³ is | difluoromethyl | trifluoromethoxy | Y¹ is CH, Y² is |
| | chloro | | | CH, Y³ is CH |
| P.0600 | X¹ is chloro, X² is N, X³ is | difluoromethyl | trifluoromethyl | Y¹ is CH, Y² is |
| | chloro | | | CH, Y³ is CH |
| P.0601 | X¹ is chloro, X² is N, X³ is | trifluoromethyl | bromo | Y¹ is CH, Y² is |
| | chloro | | | CH, Y³ is CH |
| P.0602 | X¹ is chloro, X² is N, X³ is | trifluoromethyl | chloro | Y¹ is CH, Y² is |
| | chloro | | | CH, Y³ is CH |
| P.0603 | X¹ is chloro, X² is N, X³ is | trifluoromethyl | cyano | Y¹ is CH, Y² is |
| | chloro | | | CH, Y³ is CH |
| P.0604 | X¹ is chloro, X² is N, X³ is | trifluoromethyl | cyclopropyl | Y¹ is CH, Y² is |
| | chloro | | | CH, Y³ is CH |
| P.0605 | X¹ is chloro, X² is N, X³ is | trifluoromethyl | ethyl | Y¹ is CH, Y² is |
| | chloro | | | CH, Y³ is CH |
| P.0606 | X¹ is chloro, X² is N, X³ is | trifluoromethyl | fluoro | Y¹ is CH, Y² is |
| | chloro | | | CH, Y³ is CH |
| P.0607 | X¹ is chloro, X² is N, X³ is | trifluoromethyl | hydrogen | Y¹ is CH, Y² is |
| | chloro | | | CH, Y³ is CH |
| P.0608 | X¹ is chloro, X² is N, X³ is | trifluoromethyl | methoxy | Y¹ is CH, Y² is |
| | chloro | | | CH, Y³ is CH |
| P.0609 | X¹ is chloro, X² is N, X³ is | trifluoromethyl | methyl | Y¹ is CH, Y² is |
| | chloro | | | CH, Y³ is CH |
| P.0610 | X¹ is chloro, X² is N, X³ is | trifluoromethyl | nitro | Y¹ is CH, Y² is |
| | chloro | | | CH, Y³ is CH |
| P.0611 | X¹ is chloro, X² is N, X³ is | trifluoromethyl | trifluoromethoxy | Y¹ is CH, Y² is |
| | chloro | | | CH, Y³ is CH |
| P.0612 | X¹ is chloro, X² is N, X³ is | trifluoromethyl | trifluoromethyl | Y¹ is CH, Y² is |
| | chloro | | | CH, Y³ is CH |
| P.0613 | X¹ is trifluoromethyl, X² is | chlorodifluoromethyl | bromo | Y¹ is CH, Y² is |
| | N, X³ is trifluoromethyl | | | CH, Y³ is CH |
| P.0614 | X¹ is trifluoromethyl, X² is | chlorodifluoromethyl | chloro | Y¹ is CH, Y² is |
| | N, X³ is trifluoromethyl | | | CH, Y³ is CH |
| P.0615 | X¹ is trifluoromethyl, X² is | chlorodifluoromethyl | cyano | Y¹ is CH, Y² is |
| | N, X³ is trifluoromethyl | | | CH, Y³ is CH |
| P.0616 | X¹ is trifluoromethyl, X² is | chlorodifluoromethyl | cyclopropyl | Y¹ is CH, Y² is |
| | N, X³ is trifluoromethyl | | | CH, Y³ is CH |
| P.0617 | X¹ is trifluoromethyl, X² is | chlorodifluoromethyl | ethyl | Y¹ is CH, Y² is |
| | N, X³ is trifluoromethyl | | | CH, Y³ is CH |
| P.0618 | X¹ is trifluoromethyl, X² is | chlorodifluoromethyl | fluoro | Y¹ is CH, Y² is |
| | N, X³ is trifluoromethyl | | | CH, Y³ is CH |
| P.0619 | X¹ is trifluoromethyl, X² is | chlorodifluoromethyl | hydrogen | Y¹ is CH, Y² is |
| | N, X³ is trifluoromethyl | | | CH, Y³ is CH |
| P.0620 | X¹ is trifluoromethyl, X² is | chlorodifluoromethyl | methoxy | Y¹ is CH, Y² is |
| | N, X³ is trifluoromethyl | | | CH, Y³ is CH |
| P.0621 | X¹ is trifluoromethyl, X² is | chlorodifluoromethyl | methyl | Y¹ is CH, Y² is |
| | N, X³ is trifluoromethyl | | | CH, Y³ is CH |
| P.0622 | X¹ is trifluoromethyl, X² is | chlorodifluoromethyl | nitro | Y¹ is CH, Y² is |
| | N, X³ is trifluoromethyl | | | CH, Y³ is CH |
| P.0623 | X¹ is trifluoromethyl, X² is | chlorodifluoromethyl | trifluoromethoxy | Y¹ is CH, Y² is |
| | N, X³ is trifluoromethyl | | | CH, Y³ is CH |
| P.0624 | X¹ is trifluoromethyl, X² is | chlorodifluoromethyl | trifluoromethyl | Y¹ is CH, Y² is |
| | N, X³ is trifluoromethyl | | | CH, Y³ is CH |
| P.0625 | X¹ is trifluoromethyl, X² is | difluoromethyl | bromo | Y¹ is CH, Y² is |
| | N, X³ is trifluoromethyl | | | CH, Y³ is CH |
| P.0626 | X¹ is trifluoromethyl, X² is | difluoromethyl | chloro | Y¹ is CH, Y² is |
| | N, X³ is trifluoromethyl | | | CH, Y³ is CH |
| P.0627 | X¹ is trifluoromethyl, X² is | difluoromethyl | cyano | Y¹ is CH, Y² is |
| | N, X³ is trifluoromethyl | | | CH, Y³ is CH |
| P.0628 | X¹ is trifluoromethyl, X² is | difluoromethyl | cyclopropyl | Y¹ is CH, Y² is |
| | N, X³ is trifluoromethyl | | | CH, Y³ is CH |
| P.0629 | X¹ is trifluoromethyl, X² is | difluoromethyl | ethyl | Y¹ is CH, Y² is |
| | N, X³ is trifluoromethyl | | | CH, Y³ is CH |
| P.0630 | X¹ is trifluoromethyl, X² is | difluoromethyl | fluoro | Y¹ is CH, Y² is |
| | N, X³ is trifluoromethyl | | | CH, Y³ is CH |
| P.0631 | X¹ is trifluoromethyl, X² is | difluoromethyl | hydrogen | Y¹ is CH, Y² is |
| | N, X³ is trifluoromethyl | | | CH, Y³ is CH |
| P.0632 | X¹ is trifluoromethyl, X² is | difluoromethyl | methoxy | Y¹ is CH, Y² is |
| | N, X³ is trifluoromethyl | | | CH, Y³ is CH |
| P.0633 | X¹ is trifluoromethyl, X² is | difluoromethyl | methyl | Y¹ is CH, Y² is |
| | N, X³ is trifluoromethyl | | | CH, Y³ is CH |
| P.0634 | X¹ is trifluoromethyl, X² is | difluoromethyl | nitro | Y¹ is CH, Y² is |
| | N, X³ is trifluoromethyl | | | CH, Y³ is CH |
| P.0635 | X¹ is trifluoromethyl, X² is | difluoromethyl | trifluoromethoxy | Y¹ is CH, Y² is |
| | N, X³ is trifluoromethyl | | | CH, Y³ is CH |
| P.0636 | X¹ is trifluoromethyl, X² is | difluoromethyl | trifluoromethyl | Y¹ is CH, Y² is |
| | N, X³ is trifluoromethyl | | | CH, Y³ is CH |
| P.0637 | X¹ is trifluoromethyl, X² is | trifluoromethyl | bromo | Y¹ is CH, Y² is |
| | N, X³ is trifluoromethyl | | | CH, Y³ is CH |
| P.0638 | X¹ is trifluoromethyl, X² is | trifluoromethyl | chloro | Y¹ is CH, Y² is |
| | N, X³ is trifluoromethyl | | | CH, Y³ is CH |
| P.0639 | X¹ is trifluoromethyl, X² is | trifluoromethyl | cyano | Y¹ is CH, Y² is |
| | N, X³ is trifluoromethyl | | | CH, Y³ is CH |
| P.0640 | X¹ is trifluoromethyl, X² is | trifluoromethyl | cyclopropyl | Y¹ is CH, Y² is |
| | N, X³ is trifluoromethyl | | | CH, Y³ is CH |
| P.0641 | X¹ is trifluoromethyl, X² is | trifluoromethyl | ethyl | Y¹ is CH, Y² is |
| | N, X³ is trifluoromethyl | | | CH, Y³ is CH |
| P.0642 | X¹ is trifluoromethyl, X² is | trifluoromethyl | fluoro | Y¹ is CH, Y² is |
| | N, X³ is trifluoromethyl | | | CH, Y³ is CH |
| P.0643 | X¹ is trifluoromethyl, X² is | trifluoromethyl | hydrogen | Y¹ is CH, Y² is |
| | N, X³ is trifluoromethyl | | | CH, Y³ is CH |
| P.0644 | X¹ is trifluoromethyl, X² is | trifluoromethyl | methoxy | Y¹ is CH, Y² is |
| | N, X³ is trifluoromethyl | | | CH, Y³ is CH |
| P.0645 | X¹ is trifluoromethyl, X² is | trifluoromethyl | methyl | Y¹ is CH, Y² is |
| | N, X³ is trifluoromethyl | | | CH, Y³ is CH |
| P.0646 | X¹ is trifluoromethyl, X² is | trifluoromethyl | nitro | Y¹ is CH, Y² is |
| | N, X³ is trifluoromethyl | | | CH, Y³ is CH |
| P.0647 | X¹ is trifluoromethyl, X² is | trifluoromethyl | trifluoromethoxy | Y¹ is CH, Y² is |
| | N, X³ is trifluoromethyl | | | CH, Y³ is CH |
| P.0648 | X¹ is trifluoromethyl, X² is | trifluoromethyl | trifluoromethyl | Y¹ is CH, Y² is |
| | N, X³ is trifluoromethyl | | | CH, Y³ is CH |
| P.0649 | X¹ is chloro, X² is CH, X³ is | chlorodifluoromethyl | bromo | Y¹ is N, Y² is CH, |
| | chloro | | | Y³ is CH |
| P.0650 | X¹ is chloro, X² is CH, X³ is | chlorodifluoromethyl | chloro | Y¹ is N, Y² is CH, |
| | chloro | | | Y³ is CH |
| P.0651 | X¹ is chloro, X² is CH, X³ is | chlorodifluoromethyl | cyano | Y¹ is N, Y² is CH, |
| | chloro | | | Y³ is CH |
| P.0652 | X¹ is chloro, X² is CH, X³ is | chlorodifluoromethyl | cyclopropyl | Y¹ is N, Y² is CH, |
| | chloro | | | Y³ is CH |
| P.0653 | X¹ is chloro, X² is CH, X³ is | chlorodifluoromethyl | ethyl | Y¹ is N, Y² is CH, |
| | chloro | | | Y³ is CH |
| P.0654 | X¹ is chloro, X² is CH, X³ is | chlorodifluoromethyl | fluoro | Y¹ is N, Y² is CH, |
| | chloro | | | Y³ is CH |
| P.0655 | X¹ is chloro, X² is CH, X³ is | chlorodifluoromethyl | hydrogen | Y¹ is N, Y² is CH, |
| | chloro | | | Y³ is CH |
| P.0656 | X¹ is chloro, X² is CH, X³ is | chlorodifluoromethyl | methoxy | Y¹ is N, Y² is CH, |
| | chloro | | | Y³ is CH |
| P.0657 | X¹ is chloro, X² is CH, X³ is | chlorodifluoromethyl | methyl | Y¹ is N, Y² is CH, |
| | chloro | | | Y³ is CH |
| P.0658 | X¹ is chloro, X² is CH, X³ is | chlorodifluoromethyl | nitro | Y¹ is N, Y² is CH, |
| | chloro | | | Y³ is CH |
| P.0659 | X¹ is chloro, X² is CH, X³ is | chlorodifluoromethyl | trifluoromethoxy | Y¹ is N, Y² is CH, |
| | chloro | | | Y³ is CH |
| P.0660 | X¹ is chloro, X² is CH, X³ is | chlorodifluoromethyl | trifluoromethyl | Y¹ is N, Y² is CH, |
| | chloro | | | Y³ is CH |
| P.0661 | X¹ is chloro, X² is CH, X³ is | difluoromethyl | bromo | Y¹ is N, Y² is CH, |
| | chloro | | | Y³ is CH |
| P.0662 | X¹ is chloro, X² is CH, X³ is | difluoromethyl | chloro | Y¹ is N, Y² is CH, |
| | chloro | | | Y³ is CH |
| P.0663 | X¹ is chloro, X² is CH, X³ is | difluoromethyl | cyano | Y¹ is N, Y² is CH, |
| | chloro | | | Y³ is CH |
| P.0664 | X¹ is chloro, X² is CH, X³ is | difluoromethyl | cyclopropyl | Y¹ is N, Y² is CH, |
| | chloro | | | Y³ is CH |
| P.0665 | X¹ is chloro, X² is CH, X³ is | difluoromethyl | ethyl | Y¹ is N, Y² is CH, |
| | chloro | | | Y³ is CH |
| P.0666 | X¹ is chloro, X² is CH, X³ is | difluoromethyl | fluoro | Y¹ is N, Y² is CH, |
| | chloro | | | Y³ is CH |
| P.0667 | X¹ is chloro, X² is CH, X³ is | difluoromethyl | hydrogen | Y¹ is N, Y² is CH, |
| | chloro | | | Y³ is CH |
| P.0668 | X¹ is chloro, X² is CH, X³ is | difluoromethyl | methoxy | Y¹ is N, Y² is CH, |
| | chloro | | | Y³ is CH |
| P.0669 | X¹ is chloro, X² is CH, X³ is | difluoromethyl | methyl | Y¹ is N, Y² is CH, |
| | chloro | | | Y³ is CH |
| P.0670 | X¹ is chloro, X² is CH, X³ is | difluoromethyl | nitro | Y¹ is N, Y² is CH, |
| | chloro | | | Y³ is CH |
| P.0671 | X¹ is chloro, X² is CH, X³ is | difluoromethyl | trifluoromethoxy | Y¹ is N, Y² is CH, |
| | chloro | | | Y³ is CH |
| P.0672 | X¹ is chloro, X² is CH, X³ is | difluoromethyl | trifluoromethyl | Y¹ is N, Y² is CH, |
| | chloro | | | Y³ is CH |
| P.0673 | X¹ is chloro, X² is CH, X³ is | trifluoromethyl | bromo | Y¹ is N, Y² is CH, |
| | chloro | | | Y³ is CH |
| P.0674 | X¹ is chloro, X² is CH, X³ is | trifluoromethyl | chloro | Y¹ is N, Y² is CH, |
| | chloro | | | Y³ is CH |
| P.0675 | X¹ is chloro, X² is CH, X³ is | trifluoromethyl | cyano | Y¹ is N, Y² is CH, |
| | chloro | | | Y³ is CH |
| P.0676 | X¹ is chloro, X² is CH, X³ is | trifluoromethyl | cyclopropyl | Y¹ is N, Y² is CH, |
| | chloro | | | Y³ is CH |
| P.0677 | X¹ is chloro, X² is CH, X³ is | trifluoromethyl | ethyl | Y¹ is N, Y² is CH, |
| | chloro | | | Y³ is CH |
| P.0678 | X¹ is chloro, X² is CH, X³ is | trifluoromethyl | fluoro | Y¹ is N, Y² is CH, |
| | chloro | | | Y³ is CH |
| P.0679 | X¹ is chloro, X² is CH, X³ is | trifluoromethyl | hydrogen | Y¹ is N, Y² is CH, |
| | chloro | | | Y³ is CH |
| P.0680 | X¹ is chloro, X² is CH, X³ is | trifluoromethyl | methoxy | Y¹ is N, Y² is CH, |
| | chloro | | | Y³ is CH |
| P.0681 | X¹ is chloro, X² is CH, X³ is | trifluoromethyl | methyl | Y¹ is N, Y² is CH, |
| | chloro | | | Y³ is CH |
| P.0682 | X¹ is chloro, X² is CH, X³ is | trifluoromethyl | nitro | Y¹ is N, Y² is CH, |
| | chloro | | | Y³ is CH |
| P.0683 | X¹ is chloro, X² is CH, X³ is | trifluoromethyl | trifluoromethoxy | Y¹ is N, Y² is CH, |
| | chloro | | | Y³ is CH |
| P.0684 | X¹ is chloro, X² is CH, X³ is | trifluoromethyl | trifluoromethyl | Y¹ is N, Y² is CH, |
| | chloro | | | Y³ is CH |
| P.0685 | X¹ is chloro, X² is C—F, X³ is | chlorodifluoromethyl | bromo | Y¹ is N, Y² is CH, |
| | hydrogen | | | Y³ is CH |
| P.0686 | X¹ is chloro, X² is C—F, X³ is | chlorodifluoromethyl | chloro | Y¹ is N, Y² is CH, |
| | hydrogen | | | Y³ is CH |
| P.0687 | X¹ is chloro, X² is C—F, X³ is | chlorodifluoromethyl | cyano | Y¹ is N, Y² is CH, |
| | hydrogen | | | Y³ is CH |
| P.0688 | X¹ is chloro, X² is C—F, X³ is | chlorodifluoromethyl | cyclopropyl | Y¹ is N, Y² is CH, |
| | hydrogen | | | Y³ is CH |
| P.0689 | X¹ is chloro, X² is C—F, X³ is | chlorodifluoromethyl | ethyl | Y¹ is N, Y² is CH, |
| | hydrogen | | | Y³ is CH |
| P.0690 | X¹ is chloro, X² is C—F, X³ is | chlorodifluoromethyl | fluoro | Y¹ is N, Y² is CH, |
| | hydrogen | | | Y³ is CH |
| P.0691 | X¹ is chloro, X² is C—F, X³ is | chlorodifluoromethyl | hydrogen | Y¹ is N, Y² is CH, |
| | hydrogen | | | Y³ is CH |
| P.0692 | X¹ is chloro, X² is C—F, X³ is | chlorodifluoromethyl | methoxy | Y¹ is N, Y² is CH, |
| | hydrogen | | | Y³ is CH |
| P.0693 | X¹ is chloro, X² is C—F, X³ is | chlorodifluoromethyl | methyl | Y¹ is N, Y² is CH, |
| | hydrogen | | | Y³ is CH |
| P.0694 | X¹ is chloro, X² is C—F, X³ is | chlorodifluoromethyl | nitro | Y¹ is N, Y² is CH, |
| | hydrogen | | | Y³ is CH |
| P.0695 | X¹ is chloro, X² is C—F, X³ is | chlorodifluoromethyl | trifluoromethoxy | Y¹ is N, Y² is CH, |
| | hydrogen | | | Y³ is CH |
| P.0696 | X¹ is chloro, X² is C—F, X³ is | chlorodifluoromethyl | trifluoromethyl | Y¹ is N, Y² is CH, |
| | hydrogen | | | Y³ is CH |
| P.0697 | X¹ is chloro, X² is C—F, X³ is | difluoromethyl | bromo | Y¹ is N, Y² is CH, |
| | hydrogen | | | Y³ is CH |
| P.0698 | X¹ is chloro, X² is C—F, X³ is | difluoromethyl | chloro | Y¹ is N, Y² is CH, |
| | hydrogen | | | Y³ is CH |
| P.0699 | X¹ is chloro, X² is C—F, X³ is | difluoromethyl | cyano | Y¹ is N, Y² is CH, |
| | hydrogen | | | Y³ is CH |
| P.0700 | X¹ is chloro, X² is C—F, X³ is | difluoromethyl | cyclopropyl | Y¹ is N, Y² is CH, |
| | hydrogen | | | Y³ is CH |
| P.0701 | X¹ is chloro, X² is C—F, X³ is | difluoromethyl | ethyl | Y¹ is N, Y² is CH, |
| | hydrogen | | | Y³ is CH |
| P.0702 | X¹ is chloro, X² is C—F, X³ is | difluoromethyl | fluoro | Y¹ is N, Y² is CH, |
| | hydrogen | | | Y³ is CH |
| P.0703 | X¹ is chloro, X² is C—F, X³ is | difluoromethyl | hydrogen | Y¹ is N, Y² is CH, |
| | hydrogen | | | Y³ is CH |
| P.0704 | X¹ is chloro, X² is C—F, X³ is | difluoromethyl | methoxy | Y¹ is N, Y² is CH, |
| | hydrogen | | | Y³ is CH |
| P.0705 | X¹ is chloro, X² is C—F, X³ is | difluoromethyl | methyl | Y¹ is N, Y² is CH, |
| | hydrogen | | | Y³ is CH |
| P.0706 | X¹ is chloro, X² is C—F, X³ is | difluoromethyl | nitro | Y¹ is N, Y² is CH, |
| | hydrogen | | | Y³ is CH |
| P.0707 | X¹ is chloro, X² is C—F, X³ is | difluoromethyl | trifluoromethoxy | Y¹ is N, Y² is CH, |
| | hydrogen | | | Y³ is CH |
| P.0708 | X¹ is chloro, X² is C—F, X³ is | difluoromethyl | trifluoromethyl | Y¹ is N, Y² is CH, |
| | hydrogen | | | Y³ is CH |
| P.0709 | X¹ is chloro, X² is C—F, X³ is | trifluoromethyl | bromo | Y¹ is N, Y² is CH, |
| | hydrogen | | | Y³ is CH |
| P.0710 | X¹ is chloro, X² is C—F, X³ is | trifluoromethyl | chloro | Y¹ is N, Y² is CH, |
| | hydrogen | | | Y³ is CH |
| P.0711 | X¹ is chloro, X² is C—F, X³ is | trifluoromethyl | cyano | Y¹ is N, Y² is CH, |
| | hydrogen | | | Y³ is CH |
| P.0712 | X¹ is chloro, X² is C—F, X³ is | trifluoromethyl | cyclopropyl | Y¹ is N, Y² is CH, |
| | hydrogen | | | Y³ is CH |
| P.0713 | X¹ is chloro, X² is C—F, X³ is | trifluoromethyl | ethyl | Y¹ is N, Y² is CH, |
| | hydrogen | | | Y³ is CH |
| P.0714 | X¹ is chloro, X² is C—F, X³ is | trifluoromethyl | fluoro | Y¹ is N, Y² is CH, |
| | hydrogen | | | Y³ is CH |
| P.0715 | X¹ is chloro, X² is C—F, X³ is | trifluoromethyl | hydrogen | Y¹ is N, Y² is CH, |
| | hydrogen | | | Y³ is CH |
| P.0716 | X¹ is chloro, X² is C—F, X³ is | trifluoromethyl | methoxy | Y¹ is N, Y² is CH, |
| | hydrogen | | | Y³ is CH |
| P.0717 | X¹ is chloro, X² is C—F, X³ is | trifluoromethyl | methyl | Y¹ is N, Y² is CH, |
| | hydrogen | | | Y³ is CH |
| P.0718 | X¹ is chloro, X² is C—F, X³ is | trifluoromethyl | nitro | Y¹ is N, Y² is CH, |
| | hydrogen | | | Y³ is CH |
| P.0719 | X¹ is chloro, X² is C—F, X³ is | trifluoromethyl | trifluoromethoxy | Y¹ is N, Y² is CH, |
| | hydrogen | | | Y³ is CH |
| P.0720 | X¹ is chloro, X² is C—F, X³ is | trifluoromethyl | trifluoromethyl | Y¹ is N, Y² is CH, |
| | hydrogen | | | Y³ is CH |
| P.0721 | X¹ is fluoro, X² is C—Cl, X³ | chlorodifluoromethyl | bromo | Y¹ is N, Y² is CH, |
| | is hydrogen | | | Y³ is CH |
| P.0722 | X¹ is fluoro, X² is C—Cl, X³ | chlorodifluoromethyl | chloro | Y¹ is N, Y² is CH, |
| | is hydrogen | | | Y³ is CH |
| P.0723 | X¹ is fluoro, X² is C—Cl, X³ | chlorodifluoromethyl | cyano | Y¹ is N, Y² is CH, |
| | is hydrogen | | | Y³ is CH |
| P.0724 | X¹ is fluoro, X² is C—Cl, X³ | chlorodifluoromethyl | cyclopropyl | Y¹ is N, Y² is CH, |
| | is hydrogen | | | Y³ is CH |
| P.0725 | X¹ is fluoro, X² is C—Cl, X³ | chlorodifluoromethyl | ethyl | Y¹ is N, Y² is CH, |
| | is hydrogen | | | Y³ is CH |
| P.0726 | X¹ is fluoro, X² is C—Cl, X³ | chlorodifluoromethyl | fluoro | Y¹ is N, Y² is CH, |
| | is hydrogen | | | Y³ is CH |
| P.0727 | X¹ is fluoro, X² is C—Cl, X³ | chlorodifluoromethyl | hydrogen | Y¹ is N, Y² is CH, |
| | is hydrogen | | | Y³ is CH |
| P.0728 | X¹ is fluoro, X² is C—Cl, X³ | chlorodifluoromethyl | methoxy | Y¹ is N, Y² is CH, |
| | is hydrogen | | | Y³ is CH |
| P.0729 | X¹ is fluoro, X² is C—Cl, X³ | chlorodifluoromethyl | methyl | Y¹ is N, Y² is CH, |
| | is hydrogen | | | Y³ is CH |
| P.0730 | X¹ is fluoro, X² is C—Cl, X³ | chlorodifluoromethyl | nitro | Y¹ is N, Y² is CH, |
| | is hydrogen | | | Y³ is CH |
| P.0731 | X¹ is fluoro, X² is C—Cl, X³ | chlorodifluoromethyl | trifluoromethoxy | Y¹ is N, Y² is CH, |
| | is hydrogen | | | Y³ is CH |
| P.0732 | X¹ is fluoro, X² is C—Cl, X³ | chlorodifluoromethyl | trifluoromethyl | Y¹ is N, Y² is CH, |
| | is hydrogen | | | Y³ is CH |
| P.0733 | X¹ is fluoro, X² is C—Cl, X³ | difluoromethyl | bromo | Y¹ is N, Y² is CH, |
| | is hydrogen | | | Y³ is CH |
| P.0734 | X¹ is fluoro, X² is C—Cl, X³ | difluoromethyl | chloro | Y¹ is N, Y² is CH, |
| | is hydrogen | | | Y³ is CH |
| P.0735 | X¹ is fluoro, X² is C—Cl, X³ | difluoromethyl | cyano | Y¹ is N, Y² is CH, |
| | is hydrogen | | | Y³ is CH |
| P.0736 | X¹ is fluoro, X² is C—Cl, X³ | difluoromethyl | cyclopropyl | Y¹ is N, Y² is CH, |
| | is hydrogen | | | Y³ is CH |
| P.0737 | X¹ is fluoro, X² is C—Cl, X³ | difluoromethyl | ethyl | Y¹ is N, Y² is CH, |
| | is hydrogen | | | Y³ is CH |
| P.0738 | X¹ is fluoro, X² is C—Cl, X³ | difluoromethyl | fluoro | Y¹ is N, Y² is CH, |
| | is hydrogen | | | Y³ is CH |
| P.0739 | X¹ is fluoro, X² is C—Cl, X³ | difluoromethyl | hydrogen | Y¹ is N, Y² is CH, |
| | is hydrogen | | | Y³ is CH |
| P.0740 | X¹ is fluoro, X² is C—Cl, X³ | difluoromethyl | methoxy | Y¹ is N, Y² is CH, |
| | is hydrogen | | | Y³ is CH |
| P.0741 | X¹ is fluoro, X² is C—Cl, X³ | difluoromethyl | methyl | Y¹ is N, Y² is CH, |
| | is hydrogen | | | Y³ is CH |
| P.0742 | X¹ is fluoro, X² is C—Cl, X³ | difluoromethyl | nitro | Y¹ is N, Y² is CH, |
| | is hydrogen | | | Y³ is CH |
| P.0743 | X¹ is fluoro, X² is C—Cl, X³ | difluoromethyl | trifluoromethoxy | Y¹ is N, Y² is CH, |
| | is hydrogen | | | Y³ is CH |
| P.0744 | X¹ is fluoro, X² is C—Cl, X³ | difluoromethyl | trifluoromethyl | Y¹ is N, Y² is CH, |
| | is hydrogen | | | Y³ is CH |
| P.0745 | X¹ is fluoro, X² is C—Cl, X³ | trifluoromethyl | bromo | Y¹ is N, Y² is CH, |
| | is hydrogen | | | Y³ is CH |
| P.0746 | X¹ is fluoro, X² is C—Cl, X³ | trifluoromethyl | chloro | Y¹ is N, Y² is CH, |
| | is hydrogen | | | Y³ is CH |
| P.0747 | X¹ is fluoro, X² is C—Cl, X³ | trifluoromethyl | cyano | Y¹ is N, Y² is CH, |
| | is hydrogen | | | Y³ is CH |
| P.0748 | X¹ is fluoro, X² is C—Cl, X³ | trifluoromethyl | cyclopropyl | Y¹ is N, Y² is CH, |
| | is hydrogen | | | Y³ is CH |
| P.0749 | X¹ is fluoro, X² is C—Cl, X³ | trifluoromethyl | ethyl | Y¹ is N, Y² is CH, |
| | is hydrogen | | | Y³ is CH |
| P.0750 | X¹ is fluoro, X² is C—Cl, X³ | trifluoromethyl | fluoro | Y¹ is N, Y² is CH, |
| | is hydrogen | | | Y³ is CH |
| P.0751 | X¹ is fluoro, X² is C—Cl, X³ | trifluoromethyl | hydrogen | Y¹ is N, Y² is CH, |
| | is hydrogen | | | Y³ is CH |
| P.0752 | X¹ is fluoro, X² is C—Cl, X³ | trifluoromethyl | methoxy | Y¹ is N, Y² is CH, |
| | is hydrogen | | | Y³ is CH |
| P.0753 | X¹ is fluoro, X² is C—Cl, X³ | trifluoromethyl | methyl | Y¹ is N, Y² is CH, |
| | is hydrogen | | | Y³ is CH |
| P.0754 | X¹ is fluoro, X² is C—Cl, X³ | trifluoromethyl | nitro | Y¹ is N, Y² is CH, |
| | is hydrogen | | | Y³ is CH |
| P.0755 | X¹ is fluoro, X² is C—Cl, X³ | trifluoromethyl | trifluoromethoxy | Y¹ is N, Y² is CH, |
| | is hydrogen | | | Y³ is CH |
| P.0756 | X¹ is fluoro, X² is C—Cl, X³ | trifluoromethyl | trifluoromethyl | Y¹ is N, Y² is CH, |
| | is hydrogen | | | Y³ is CH |
| P.0757 | X¹ is chloro, X² is C—Cl, X³ | chlorodifluoromethyl | bromo | Y¹ is N, Y² is CH, |
| | is hydrogen | | | Y³ is CH |
| P.0758 | X¹ is chloro, X² is C—Cl, X³ | chlorodifluoromethyl | chloro | Y¹ is N, Y² is CH, |
| | is hydrogen | | | Y³ is CH |
| P.0759 | X¹ is chloro, X² is C—Cl, X³ | chlorodifluoromethyl | cyano | Y¹ is N, Y² is CH, |
| | is hydrogen | | | Y³ is CH |
| P.0760 | X¹ is chloro, X² is C—Cl, X³ | chlorodifluoromethyl | cyclopropyl | Y¹ is N, Y² is CH, |
| | is hydrogen | | | Y³ is CH |
| P.0761 | X¹ is chloro, X² is C—Cl, X³ | chlorodifluoromethyl | ethyl | Y¹ is N, Y² is CH, |
| | is hydrogen | | | Y³ is CH |
| P.0762 | X¹ is chloro, X² is C—Cl, X³ | chlorodifluoromethyl | fluoro | Y¹ is N, Y² is CH, |
| | is hydrogen | | | Y³ is CH |
| P.0763 | X¹ is chloro, X² is C—Cl, X³ | chlorodifluoromethyl | hydrogen | Y¹ is N, Y² is CH, |
| | is hydrogen | | | Y³ is CH |
| P.0764 | X¹ is chloro, X² is C—Cl, X³ | chlorodifluoromethyl | methoxy | Y¹ is N, Y² is CH, |
| | is hydrogen | | | Y³ is CH |
| P.0765 | X¹ is chloro, X² is C—Cl, X³ | chlorodifluoromethyl | methyl | Y¹ is N, Y² is CH, |
| | is hydrogen | | | Y³ is CH |
| P.0766 | X¹ is chloro, X² is C—Cl, X³ | chlorodifluoromethyl | nitro | Y¹ is N, Y² is CH, |
| | is hydrogen | | | Y³ is CH |
| P.0767 | X¹ is chloro, X² is C—Cl, X³ | chlorodifluoromethyl | trifluoromethoxy | Y¹ is N, Y² is CH, |
| | is hydrogen | | | Y³ is CH |
| P.0768 | X¹ is chloro, X² is C—Cl, X³ | chlorodifluoromethyl | trifluoromethyl | Y¹ is N, Y² is CH, |
| | is hydrogen | | | Y³ is CH |
| P.0769 | X¹ is chloro, X² is C—Cl, X³ | difluoromethyl | bromo | Y¹ is N, Y² is CH, |
| | is hydrogen | | | Y³ is CH |
| P.0770 | X¹ is chloro, X² is C—Cl, X³ | difluoromethyl | chloro | Y¹ is N, Y² is CH, |
| | is hydrogen | | | Y³ is CH |
| P.0771 | X¹ is chloro, X² is C—Cl, X³ | difluoromethyl | cyano | Y¹ is N, Y² is CH, |
| | is hydrogen | | | Y³ is CH |
| P.0772 | X¹ is chloro, X² is C—Cl, X³ | difluoromethyl | cyclopropyl | Y¹ is N, Y² is CH, |
| | is hydrogen | | | Y³ is CH |
| P.0773 | X¹ is chloro, X² is C—Cl, X³ | difluoromethyl | ethyl | Y¹ is N, Y² is CH, |
| | is hydrogen | | | Y³ is CH |
| P.0774 | X¹ is chloro, X² is C—Cl, X³ | difluoromethyl | fluoro | Y¹ is N, Y² is CH, |
| | is hydrogen | | | Y³ is CH |
| P.0775 | X¹ is chloro, X² is C—Cl, X³ | difluoromethyl | hydrogen | Y¹ is N, Y² is CH, |
| | is hydrogen | | | Y³ is CH |
| P.0776 | X¹ is chloro, X² is C—Cl, X³ | difluoromethyl | methoxy | Y¹ is N, Y² is CH, |
| | is hydrogen | | | Y³ is CH |
| P.0777 | X¹ is chloro, X² is C—Cl, X³ | difluoromethyl | methyl | Y¹ is N, Y² is CH, |
| | is hydrogen | | | Y³ is CH |
| P.0778 | X¹ is chloro, X² is C—Cl, X³ | difluoromethyl | nitro | Y¹ is N, Y² is CH, |
| | is hydrogen | | | Y³ is CH |
| P.0779 | X¹ is chloro, X² is C—Cl, X³ | difluoromethyl | trifluoromethoxy | Y¹ is N, Y² is CH, |
| | is hydrogen | | | Y³ is CH |
| P.0780 | X¹ is chloro, X² is C—Cl, X³ | difluoromethyl | trifluoromethyl | Y¹ is N, Y² is CH, |
| | is hydrogen | | | Y³ is CH |
| P.0781 | X¹ is chloro, X² is C—Cl, X³ | trifluoromethyl | bromo | Y¹ is N, Y² is CH, |
| | is hydrogen | | | Y³ is CH |
| P.0782 | X¹ is chloro, X² is C—Cl, X³ | trifluoromethyl | chloro | Y¹ is N, Y² is CH, |
| | is hydrogen | | | Y³ is CH |
| P.0783 | X¹ is chloro, X² is C—Cl, X³ | trifluoromethyl | cyano | Y¹ is N, Y² is CH, |
| | is hydrogen | | | Y³ is CH |
| P.0784 | X¹ is chloro, X² is C—Cl, X³ | trifluoromethyl | cyclopropyl | Y¹ is N, Y² is CH, |
| | is hydrogen | | | Y³ is CH |
| P.0785 | X¹ is chloro, X² is C—Cl, X³ | trifluoromethyl | ethyl | Y¹ is N, Y² is CH, |
| | is hydrogen | | | Y³ is CH |
| P.0786 | X¹ is chloro, X² is C—Cl, X³ | trifluoromethyl | fluoro | Y¹ is N, Y² is CH, |
| | is hydrogen | | | Y³ is CH |
| P.0787 | X¹ is chloro, X² is C—Cl, X³ | trifluoromethyl | hydrogen | Y¹ is N, Y² is CH, |
| | is hydrogen | | | Y³ is CH |
| P.0788 | X¹ is chloro, X² is C—Cl, X³ | trifluoromethyl | methoxy | Y¹ is N, Y² is CH, |
| | is hydrogen | | | Y³ is CH |
| P.0789 | X¹ is chloro, X² is C—Cl, X³ | trifluoromethyl | methyl | Y¹ is N, Y² is CH, |
| | is hydrogen | | | Y³ is CH |
| P.0790 | X¹ is chloro, X² is C—Cl, X³ | trifluoromethyl | nitro | Y¹ is N, Y² is CH, |
| | is hydrogen | | | Y³ is CH |
| P.0791 | X¹ is chloro, X² is C—Cl, X³ | trifluoromethyl | trifluoromethoxy | Y¹ is N, Y² is CH, |
| | is hydrogen | | | Y³ is CH |
| P.0792 | X¹ is chloro, X² is C—Cl, X³ | trifluoromethyl | trifluoromethyl | Y¹ is N, Y² is CH, |
| | is hydrogen | | | Y³ is CH |
| P.0793 | X¹ is chloro, X² is C—Br, X³ | chlorodifluoromethyl | bromo | Y¹ is N, Y² is CH, |
| | is chloro | | | Y³ is CH |
| P.0794 | X¹ is chloro, X² is C—Br, X³ | chlorodifluoromethyl | chloro | Y¹ is N, Y² is CH, |
| | is chloro | | | Y³ is CH |
| P.0795 | X¹ is chloro, X² is C—Br, X³ | chlorodifluoromethyl | cyano | Y¹ is N, Y² is CH, |
| | is chloro | | | Y³ is CH |
| P.0796 | X¹ is chloro, X² is C—Br, X³ | chlorodifluoromethyl | cyclopropyl | Y¹ is N, Y² is CH, |
| | is chloro | | | Y³ is CH |
| P.0797 | X¹ is chloro, X² is C—Br, X³ | chlorodifluoromethyl | ethyl | Y¹ is N, Y² is CH, |
| | is chloro | | | Y³ is CH |
| P.0798 | X¹ is chloro, X² is C—Br, X³ | chlorodifluoromethyl | fluoro | Y¹ is N, Y² is CH, |
| | is chloro | | | Y³ is CH |
| P.0799 | X¹ is chloro, X² is C—Br, X³ | chlorodifluoromethyl | hydrogen | Y¹ is N, Y² is CH, |
| | is chloro | | | Y³ is CH |
| P.0800 | X¹ is chloro, X² is C—Br, X³ | chlorodifluoromethyl | methoxy | Y¹ is N, Y² is CH, |
| | is chloro | | | Y³ is CH |
| P.0801 | X¹ is chloro, X² is C—Br, X³ | chlorodifluoromethyl | methyl | Y¹ is N, Y² is CH, |
| | is chloro | | | Y³ is CH |
| P.0802 | X¹ is chloro, X² is C—Br, X³ | chlorodifluoromethyl | nitro | Y¹ is N, Y² is CH, |
| | is chloro | | | Y³ is CH |
| P.0803 | X¹ is chloro, X² is C—Br, X³ | chlorodifluoromethyl | trifluoromethoxy | Y¹ is N, Y² is CH, |
| | is chloro | | | Y³ is CH |
| P.0804 | X¹ is chloro, X² is C—Br, X³ | chlorodifluoromethyl | trifluoromethyl | Y¹ is N, Y² is CH, |
| | is chloro | | | Y³ is CH |
| P.0805 | X¹ is chloro, X² is C—Br, X³ | difluoromethyl | bromo | Y¹ is N, Y² is CH, |
| | is chloro | | | Y³ is CH |
| P.0806 | X¹ is chloro, X² is C—Br, X³ | difluoromethyl | chloro | Y¹ is N, Y² is CH, |
| | is chloro | | | Y³ is CH |
| P.0807 | X¹ is chloro, X² is C—Br, X³ | difluoromethyl | cyano | Y¹ is N, Y² is CH, |
| | is chloro | | | Y³ is CH |
| P.0808 | X¹ is chloro, X² is C—Br, X³ | difluoromethyl | cyclopropyl | Y¹ is N, Y² is CH, |
| | is chloro | | | Y³ is CH |
| P.0809 | X¹ is chloro, X² is C—Br, X³ | difluoromethyl | ethyl | Y¹ is N, Y² is CH, |
| | is chloro | | | Y³ is CH |
| P.0810 | X¹ is chloro, X² is C—Br, X³ | difluoromethyl | fluoro | Y¹ is N, Y² is CH, |
| | is chloro | | | Y³ is CH |
| P.0811 | X¹ is chloro, X² is C—Br, X³ | difluoromethyl | hydrogen | Y¹ is N, Y² is CH, |
| | is chloro | | | Y³ is CH |
| P.0812 | X¹ is chloro, X² is C—Br, X³ | difluoromethyl | methoxy | Y¹ is N, Y² is CH, |
| | is chloro | | | Y³ is CH |
| P.0813 | X¹ is chloro, X² is C—Br, X³ | difluoromethyl | methyl | Y¹ is N, Y² is CH, |
| | is chloro | | | Y³ is CH |
| P.0814 | X¹ is chloro, X² is C—Br, X³ | difluoromethyl | nitro | Y¹ is N, Y² is CH, |
| | is chloro | | | Y³ is CH |
| P.0815 | X¹ is chloro, X² is C—Br, X³ | difluoromethyl | trifluoromethoxy | Y¹ is N, Y² is CH, |
| | is chloro | | | Y³ is CH |
| P.0816 | X¹ is chloro, X² is C—Br, X³ | difluoromethyl | trifluoromethyl | Y¹ is N, Y² is CH, |
| | is chloro | | | Y³ is CH |
| P.0817 | X¹ is chloro, X² is C—Br, X³ | trifluoromethyl | bromo | Y¹ is N, Y² is CH, |
| | is chloro | | | Y³ is CH |
| P.0818 | X¹ is chloro, X² is C—Br, X³ | trifluoromethyl | chloro | Y¹ is N, Y² is CH, |
| | is chloro | | | Y³ is CH |
| P.0819 | X¹ is chloro, X² is C—Br, X³ | trifluoromethyl | cyano | Y¹ is N, Y² is CH, |
| | is chloro | | | Y³ is CH |
| P.0820 | X¹ is chloro, X² is C—Br, X³ | trifluoromethyl | cyclopropyl | Y¹ is N, Y² is CH, |
| | is chloro | | | Y³ is CH |
| P.0821 | X¹ is chloro, X² is C—Br, X³ | trifluoromethyl | ethyl | Y¹ is N, Y² is CH, |
| | is chloro | | | Y³ is CH |
| P.0822 | X¹ is chloro, X² is C—Br, X³ | trifluoromethyl | fluoro | Y¹ is N, Y² is CH, |
| | is chloro | | | Y³ is CH |
| P.0823 | X¹ is chloro, X² is C—Br, X³ | trifluoromethyl | hydrogen | Y¹ is N, Y² is CH, |
| | is chloro | | | Y³ is CH |
| P.0824 | X¹ is chloro, X² is C—Br, X³ | trifluoromethyl | methoxy | Y¹ is N, Y² is CH, |
| | is chloro | | | Y³ is CH |
| P.0825 | X¹ is chloro, X² is C—Br, X³ | trifluoromethyl | methyl | Y¹ is N, Y² is CH, |
| | is chloro | | | Y³ is CH |
| P.0826 | X¹ is chloro, X² is C—Br, X³ | trifluoromethyl | nitro | Y¹ is N, Y² is CH, |
| | is chloro | | | Y³ is CH |
| P.0827 | X¹ is chloro, X² is C—Br, X³ | trifluoromethyl | trifluoromethoxy | Y¹ is N, Y² is CH, |
| | is chloro | | | Y³ is CH |
| P.0828 | X¹ is chloro, X² is C—Br, X³ | trifluoromethyl | trifluoromethyl | Y¹ is N, Y² is CH, |
| | is chloro | | | Y³ is CH |
| P.0829 | X¹ is chloro, X² is C—F, X³ is | chlorodifluoromethyl | bromo | Y¹ is N, Y² is CH, |
| | chloro | | | Y³ is CH |
| P.0830 | X¹ is chloro, X² is C—F, X³ is | chlorodifluoromethyl | chloro | Y¹ is N, Y² is CH, |
| | chloro | | | Y³ is CH |
| P.0831 | X¹ is chloro, X² is C—F, X³ is | chlorodifluoromethyl | cyano | Y¹ is N, Y² is CH, |
| | chloro | | | Y³ is CH |
| P.0832 | X¹ is chloro, X² is C—F, X³ is | chlorodifluoromethyl | cyclopropyl | Y¹ is N, Y² is CH, |
| | chloro | | | Y³ is CH |
| P.0833 | X¹ is chloro, X² is C—F, X³ is | chlorodifluoromethyl | ethyl | Y¹ is N, Y² is CH, |
| | chloro | | | Y³ is CH |
| P.0834 | X¹ is chloro, X² is C—F, X³ is | chlorodifluoromethyl | fluoro | Y¹ is N, Y² is CH, |
| | chloro | | | Y³ is CH |
| P.0835 | X¹ is chloro, X² is C—F, X³ is | chlorodifluoromethyl | hydrogen | Y¹ is N, Y² is CH, |
| | chloro | | | Y³ is CH |
| P.0836 | X¹ is chloro, X² is C—F, X³ is | chlorodifluoromethyl | methoxy | Y¹ is N, Y² is CH, |
| | chloro | | | Y³ is CH |
| P.0837 | X¹ is chloro, X² is C—F, X³ is | chlorodifluoromethyl | methyl | Y¹ is N, Y² is CH, |
| | chloro | | | Y³ is CH |
| P.0838 | X¹ is chloro, X² is C—F, X³ is | chlorodifluoromethyl | nitro | Y¹ is N, Y² is CH, |
| | chloro | | | Y³ is CH |
| P.0839 | X¹ is chloro, X² is C—F, X³ is | chlorodifluoromethyl | trifluoromethoxy | Y¹ is N, Y² is CH, |
| | chloro | | | Y³ is CH |
| P.0840 | X¹ is chloro, X² is C—F, X³ is | chlorodifluoromethyl | trifluoromethyl | Y¹ is N, Y² is CH, |
| | chloro | | | Y³ is CH |
| P.0841 | X¹ is chloro, X² is C—F, X³ is | difluoromethyl | bromo | Y¹ is N, Y² is CH, |
| | chloro | | | Y³ is CH |
| P.0842 | X¹ is chloro, X² is C—F, X³ is | difluoromethyl | chloro | Y¹ is N, Y² is CH, |
| | chloro | | | Y³ is CH |
| P.0843 | X¹ is chloro, X² is C—F, X³ is | difluoromethyl | cyano | Y¹ is N, Y² is CH, |
| | chloro | | | Y³ is CH |
| P.0844 | X¹ is chloro, X² is C—F, X³ is | difluoromethyl | cyclopropyl | Y¹ is N, Y² is CH, |
| | chloro | | | Y³ is CH |
| P.0845 | X¹ is chloro, X² is C—F, X³ is | difluoromethyl | ethyl | Y¹ is N, Y² is CH, |
| | chloro | | | Y³ is CH |
| P.0846 | X¹ is chloro, X² is C—F, X³ is | difluoromethyl | fluoro | Y¹ is N, Y² is CH, |
| | chloro | | | Y³ is CH |
| P.0847 | X¹ is chloro, X² is C—F, X³ is | difluoromethyl | hydrogen | Y¹ is N, Y² is CH, |
| | chloro | | | Y³ is CH |
| P.0848 | X¹ is chloro, X² is C—F, X³ is | difluoromethyl | methoxy | Y¹ is N, Y² is CH, |
| | chloro | | | Y³ is CH |
| P.0849 | X¹ is chloro, X² is C—F, X³ is | difluoromethyl | methyl | Y¹ is N, Y² is CH, |
| | chloro | | | Y³ is CH |
| P.0850 | X¹ is chloro, X² is C—F, X³ is | difluoromethyl | nitro | Y¹ is N, Y² is CH, |
| | chloro | | | Y³ is CH |
| P.0851 | X¹ is chloro, X² is C—F, X³ is | difluoromethyl | trifluoromethoxy | Y¹ is N, Y² is CH, |
| | chloro | | | Y³ is CH |
| P.0852 | X¹ is chloro, X² is C—F, X³ is | difluoromethyl | trifluoromethyl | Y¹ is N, Y² is CH, |
| | chloro | | | Y³ is CH |
| P.0853 | X¹ is chloro, X² is C—F, X³ is | trifluoromethyl | bromo | Y¹ is N, Y² is CH, |
| | chloro | | | Y³ is CH |
| P.0854 | X¹ is chloro, X² is C—F, X³ is | trifluoromethyl | chloro | Y¹ is N, Y² is CH, |
| | chloro | | | Y³ is CH |
| P.0855 | X¹ is chloro, X² is C—F, X³ is | trifluoromethyl | cyano | Y¹ is N, Y² is CH, |
| | chloro | | | Y³ is CH |
| P.0856 | X¹ is chloro, X² is C—F, X³ is | trifluoromethyl | cyclopropyl | Y¹ is N, Y² is CH, |
| | chloro | | | Y³ is CH |
| P.0857 | X¹ is chloro, X² is C—F, X³ is | trifluoromethyl | ethyl | Y¹ is N, Y² is CH, |
| | chloro | | | Y³ is CH |
| P.0858 | X¹ is chloro, X² is C—F, X³ is | trifluoromethyl | fluoro | Y¹ is N, Y² is CH, |
| | chloro | | | Y³ is CH |
| P.0859 | X¹ is chloro, X² is C—F, X³ is | trifluoromethyl | hydrogen | Y¹ is N, Y² is CH, |
| | chloro | | | Y³ is CH |
| P.0860 | X¹ is chloro, X² is C—F, X³ is | trifluoromethyl | methoxy | Y¹ is N, Y² is CH, |
| | chloro | | | Y³ is CH |
| P.0861 | X¹ is chloro, X² is C—F, X³ is | trifluoromethyl | methyl | Y¹ is N, Y² is CH, |
| | chloro | | | Y³ is CH |
| P.0862 | X¹ is chloro, X² is C—F, X³ is | trifluoromethyl | nitro | Y¹ is N, Y² is CH, |
| | chloro | | | Y³ is CH |
| P.0863 | X¹ is chloro, X² is C—F, X³ is | trifluoromethyl | trifluoromethoxy | Y¹ is N, Y² is CH, |
| | chloro | | | Y³ is CH |
| P.0864 | X¹ is chloro, X² is C—F, X³ is | trifluoromethyl | trifluoromethyl | Y¹ is N, Y² is CH, |
| | chloro | | | Y³ is CH |
| P.0865 | X¹ is chloro, X² is C—Cl, X³ | chlorodifluoromethyl | bromo | Y¹ is N, Y² is CH, |
| | is chloro | | | Y³ is CH |
| P.0866 | X¹ is chloro, X² is C—Cl, X³ | chlorodifluoromethyl | chloro | Y¹ is N, Y² is CH, |
| | is chloro | | | Y³ is CH |
| P.0867 | X¹ is chloro, X² is C—Cl, X³ | chlorodifluoromethyl | cyano | Y¹ is N, Y² is CH, |
| | is chloro | | | Y³ is CH |
| P.0868 | X¹ is chloro, X² is C—Cl, X³ | chlorodifluoromethyl | cyclopropyl | Y¹ is N, Y² is CH, |
| | is chloro | | | Y³ is CH |
| P.0869 | X¹ is chloro, X² is C—Cl, X³ | chlorodifluoromethyl | ethyl | Y¹ is N, Y² is CH, |
| | is chloro | | | Y³ is CH |
| P.0870 | X¹ is chloro, X² is C—Cl, X³ | chlorodifluoromethyl | fluoro | Y¹ is N, Y² is CH, |
| | is chloro | | | Y³ is CH |
| P.0871 | X¹ is chloro, X² is C—Cl, X³ | chlorodifluoromethyl | hydrogen | Y¹ is N, Y² is CH, |
| | is chloro | | | Y³ is CH |
| P.0872 | X¹ is chloro, X² is C—Cl, X³ | chlorodifluoromethyl | methoxy | Y¹ is N, Y² is CH, |
| | is chloro | | | Y³ is CH |
| P.0873 | X¹ is chloro, X² is C—Cl, X³ | chlorodifluoromethyl | methyl | Y¹ is N, Y² is CH, |
| | is chloro | | | Y³ is CH |
| P.0874 | X¹ is chloro, X² is C—Cl, X³ | chlorodifluoromethyl | nitro | Y¹ is N, Y² is CH, |
| | is chloro | | | Y³ is CH |
| P.0875 | X¹ is chloro, X² is C—Cl, X³ | chlorodifluoromethyl | trifluoromethoxy | Y¹ is N, Y² is CH, |
| | is chloro | | | Y³ is CH |
| P.0876 | X¹ is chloro, X² is C—Cl, X³ | chlorodifluoromethyl | trifluoromethyl | Y¹ is N, Y² is CH, |
| | is chloro | | | Y³ is CH |
| P.0877 | X¹ is chloro, X² is C—Cl, X³ | difluoromethyl | bromo | Y¹ is N, Y² is CH, |
| | is chloro | | | Y³ is CH |
| P.0878 | X¹ is chloro, X² is C—Cl, X³ | difluoromethyl | chloro | Y¹ is N, Y² is CH, |
| | is chloro | | | Y³ is CH |
| P.0879 | X¹ is chloro, X² is C—Cl, X³ | difluoromethyl | cyano | Y¹ is N, Y² is CH, |
| | is chloro | | | Y³ is CH |
| P.0880 | X¹ is chloro, X² is C—Cl, X³ | difluoromethyl | cyclopropyl | Y¹ is N, Y² is CH, |
| | is chloro | | | Y³ is CH |
| P.0881 | X¹ is chloro, X² is C—Cl, X³ | difluoromethyl | ethyl | Y¹ is N, Y² is CH, |
| | is chloro | | | Y³ is CH |
| P.0882 | X¹ is chloro, X² is C—Cl, X³ | difluoromethyl | fluoro | Y¹ is N, Y² is CH, |
| | is chloro | | | Y³ is CH |
| P.0883 | X¹ is chloro, X² is C—Cl, X³ | difluoromethyl | hydrogen | Y¹ is N, Y² is CH, |
| | is chloro | | | Y³ is CH |
| P.0884 | X¹ is chloro, X² is C—Cl, X³ | difluoromethyl | methoxy | Y¹ is N, Y² is CH, |
| | is chloro | | | Y³ is CH |
| P.0885 | X¹ is chloro, X² is C—Cl, X³ | difluoromethyl | methyl | Y¹ is N, Y² is CH, |
| | is chloro | | | Y³ is CH |
| P.0886 | X¹ is chloro, X² is C—Cl, X³ | difluoromethyl | nitro | Y¹ is N, Y² is CH, |
| | is chloro | | | Y³ is CH |
| P.0887 | X¹ is chloro, X² is C—Cl, X³ | difluoromethyl | trifluoromethoxy | Y¹ is N, Y² is CH, |
| | is chloro | | | Y³ is CH |
| P.0888 | X¹ is chloro, X² is C—Cl, X³ | difluoromethyl | trifluoromethyl | Y¹ is N, Y² is CH, |
| | is chloro | | | Y³ is CH |
| P.0889 | X¹ is chloro, X² is C—Cl, X³ | trifluoromethyl | bromo | Y¹ is N, Y² is CH, |
| | is chloro | | | Y³ is CH |
| P.0890 | X¹ is chloro, X² is C—Cl, X³ | trifluoromethyl | chloro | Y¹ is N, Y² is CH, |
| | is chloro | | | Y³ is CH |
| P.0891 | X¹ is chloro, X² is C—Cl, X³ | trifluoromethyl | cyano | Y¹ is N, Y² is CH, |
| | is chloro | | | Y³ is CH |
| P.0892 | X¹ is chloro, X² is C—Cl, X³ | trifluoromethyl | cyclopropyl | Y¹ is N, Y² is CH, |
| | is chloro | | | Y³ is CH |
| P.0893 | X¹ is chloro, X² is C—Cl, X³ | trifluoromethyl | ethyl | Y¹ is N, Y² is CH, |
| | is chloro | | | Y³ is CH |
| P.0894 | X¹ is chloro, X² is C—Cl, X³ | trifluoromethyl | fluoro | Y¹ is N, Y² is CH, |
| | is chloro | | | Y³ is CH |
| P.0895 | X¹ is chloro, X² is C—Cl, X³ | trifluoromethyl | hydrogen | Y¹ is N, Y² is CH, |
| | is chloro | | | Y³ is CH |
| P.0896 | X¹ is chloro, X² is C—Cl, X³ | trifluoromethyl | methoxy | Y¹ is N, Y² is CH, |
| | is chloro | | | Y³ is CH |
| P.0897 | X¹ is chloro, X² is C—Cl, X³ | trifluoromethyl | methyl | Y¹ is N, Y² is CH, |
| | is chloro | | | Y³ is CH |
| P.0898 | X¹ is chloro, X² is C—Cl, X³ | trifluoromethyl | nitro | Y¹ is N, Y² is CH, |
| | is chloro | | | Y³ is CH |
| P.0899 | X¹ is chloro, X² is C—Cl, X³ | trifluoromethyl | trifluoromethoxy | Y¹ is N, Y² is CH, |
| | is chloro | | | Y³ is CH |
| P.0900 | X¹ is chloro, X² is C—Cl, X³ | trifluoromethyl | trifluoromethyl | Y¹ is N, Y² is CH, |
| | is chloro | | | Y³ is CH |
| P.0901 | X¹ is chloro, X² is C—I, X³ is | chlorodifluoromethyl | bromo | Y¹ is N, Y² is CH, |
| | chloro | | | Y³ is CH |
| P.0902 | X¹ is chloro, X² is C—I, X³ is | chlorodifluoromethyl | chloro | Y¹ is N, Y² is CH, |
| | chloro | | | Y³ is CH |
| P.0903 | X¹ is chloro, X² is C—I, X³ is | chlorodifluoromethyl | cyano | Y¹ is N, Y² is CH, |
| | chloro | | | Y³ is CH |
| P.0904 | X¹ is chloro, X² is C—I, X³ is | chlorodifluoromethyl | cyclopropyl | Y¹ is N, Y² is CH, |
| | chloro | | | Y³ is CH |
| P.0905 | X¹ is chloro, X² is C—I, X³ is | chlorodifluoromethyl | ethyl | Y¹ is N, Y² is CH, |
| | chloro | | | Y³ is CH |
| P.0906 | X¹ is chloro, X² is C—I, X³ is | chlorodifluoromethyl | fluoro | Y¹ is N, Y² is CH, |
| | chloro | | | Y³ is CH |
| P.0907 | X¹ is chloro, X² is C—I, X³ is | chlorodifluoromethyl | hydrogen | Y¹ is N, Y² is CH, |
| | chloro | | | Y³ is CH |
| P.0908 | X¹ is chloro, X² is C—I, X³ is | chlorodifluoromethyl | methoxy | Y¹ is N, Y² is CH, |
| | chloro | | | Y³ is CH |
| P.0909 | X¹ is chloro, X² is C—I, X³ is | chlorodifluoromethyl | methyl | Y¹ is N, Y² is CH, |
| | chloro | | | Y³ is CH |
| P.0910 | X¹ is chloro, X² is C—I, X³ is | chlorodifluoromethyl | nitro | Y¹ is N, Y² is CH, |
| | chloro | | | Y³ is CH |
| P.0911 | X¹ is chloro, X² is C—I, X³ is | chlorodifluoromethyl | trifluoromethoxy | Y¹ is N, Y² is CH, |
| | chloro | | | Y³ is CH |
| P.0912 | X¹ is chloro, X² is C—I, X³ is | chlorodifluoromethyl | trifluoromethyl | Y¹ is N, Y² is CH, |
| | chloro | | | Y³ is CH |
| P.0913 | X¹ is chloro, X² is C—I, X³ is | difluoromethyl | bromo | Y¹ is N, Y² is CH, |
| | chloro | | | Y³ is CH |
| P.0914 | X¹ is chloro, X² is C—I, X³ is | difluoromethyl | chloro | Y¹ is N, Y² is CH, |
| | chloro | | | Y³ is CH |
| P.0915 | X¹ is chloro, X² is C—I, X³ is | difluoromethyl | cyano | Y¹ is N, Y² is CH, |
| | chloro | | | Y³ is CH |
| P.0916 | X¹ is chloro, X² is C—I, X³ is | difluoromethyl | cyclopropyl | Y¹ is N, Y² is CH, |
| | chloro | | | Y³ is CH |
| P.0917 | X¹ is chloro, X² is C—I, X³ is | difluoromethyl | ethyl | Y¹ is N, Y² is CH, |
| | chloro | | | Y³ is CH |
| P.0918 | X¹ is chloro, X² is C—I, X³ is | difluoromethyl | fluoro | Y¹ is N, Y² is CH, |
| | chloro | | | Y³ is CH |
| P.0919 | X¹ is chloro, X² is C—I, X³ is | difluoromethyl | hydrogen | Y¹ is N, Y² is CH, |
| | chloro | | | Y³ is CH |
| P.0920 | X¹ is chloro, X² is C—I, X³ is | difluoromethyl | methoxy | Y¹ is N, Y² is CH, |
| | chloro | | | Y³ is CH |
| P.0921 | X¹ is chloro, X² is C—I, X³ is | difluoromethyl | methyl | Y¹ is N, Y² is CH, |
| | chloro | | | Y³ is CH |
| P.0922 | X¹ is chloro, X² is C—I, X³ is | difluoromethyl | nitro | Y¹ is N, Y² is CH, |
| | chloro | | | Y³ is CH |
| P.0923 | X¹ is chloro, X² is C—I, X³ is | difluoromethyl | trifluoromethoxy | Y¹ is N, Y² is CH, |
| | chloro | | | Y³ is CH |
| P.0924 | X¹ is chloro, X² is C—I, X³ is | difluoromethyl | trifluoromethyl | Y¹ is N, Y² is CH, |
| | chloro | | | Y³ is CH |
| P.0925 | X¹ is chloro, X² is C—I, X³ is | trifluoromethyl | bromo | Y¹ is N, Y² is CH, |
| | chloro | | | Y³ is CH |
| P.0926 | X¹ is chloro, X² is C—I, X³ is | trifluoromethyl | chloro | Y¹ is N, Y² is CH, |
| | chloro | | | Y³ is CH |
| P.0927 | X¹ is chloro, X² is C—I, X³ is | trifluoromethyl | cyano | Y¹ is N, Y² is CH, |
| | chloro | | | Y³ is CH |
| P.0928 | X¹ is chloro, X² is C—I, X³ is | trifluoromethyl | cyclopropyl | Y¹ is N, Y² is CH, |
| | chloro | | | Y³ is CH |
| P.0929 | X¹ is chloro, X² is C—I, X³ is | trifluoromethyl | ethyl | Y¹ is N, Y² is CH, |
| | chloro | | | Y³ is CH |
| P.0930 | X¹ is chloro, X² is C—I, X³ is | trifluoromethyl | fluoro | Y¹ is N, Y² is CH, |
| | chloro | | | Y³ is CH |
| P.0931 | X¹ is chloro, X² is C—I, X³ is | trifluoromethyl | hydrogen | Y¹ is N, Y² is CH, |
| | chloro | | | Y³ is CH |
| P.0932 | X¹ is chloro, X² is C—I, X³ is | trifluoromethyl | methoxy | Y¹ is N, Y² is CH, |
| | chloro | | | Y³ is CH |
| P.0933 | X¹ is chloro, X² is C—I, X³ is | trifluoromethyl | methyl | Y¹ is N, Y² is CH, |
| | chloro | | | Y³ is CH |
| P.0934 | X¹ is chloro, X² is C—I, X³ is | trifluoromethyl | nitro | Y¹ is N, Y² is CH, |
| | chloro | | | Y³ is CH |
| P.0935 | X¹ is chloro, X² is C—I, X³ is | trifluoromethyl | trifluoromethoxy | Y¹ is N, Y² is CH, |
| | chloro | | | Y³ is CH |
| P.0936 | X¹ is chloro, X² is C—I, X³ is | trifluoromethyl | trifluoromethyl | Y¹ is N, Y² is CH, |
| | chloro | | | Y³ is CH |
| P.0937 | X¹ is fluoro, X² is C—F, X³ is | chlorodifluoromethyl | bromo | Y¹ is N, Y² is CH, |
| | fluoro | | | Y³ is CH |
| P.0938 | X¹ is fluoro, X² is C—F, X³ is | chlorodifluoromethyl | chloro | Y¹ is N, Y² is CH, |
| | fluoro | | | Y³ is CH |
| P.0939 | X¹ is fluoro, X² is C—F, X³ is | chlorodifluoromethyl | cyano | Y¹ is N, Y² is CH, |
| | fluoro | | | Y³ is CH |
| P.0940 | X¹ is fluoro, X² is C—F, X³ is | chlorodifluoromethyl | cyclopropyl | Y¹ is N, Y² is CH, |
| | fluoro | | | Y³ is CH |
| P.0941 | X¹ is fluoro, X² is C—F, X³ is | chlorodifluoromethyl | ethyl | Y¹ is N, Y² is CH, |
| | fluoro | | | Y³ is CH |
| P.0942 | X¹ is fluoro, X² is C—F, X³ is | chlorodifluoromethyl | fluoro | Y¹ is N, Y² is CH, |
| | fluoro | | | Y³ is CH |
| P.0943 | X¹ is fluoro, X² is C—F, X³ is | chlorodifluoromethyl | hydrogen | Y¹ is N, Y² is CH, |
| | fluoro | | | Y³ is CH |
| P.0944 | X¹ is fluoro, X² is C—F, X³ is | chlorodifluoromethyl | methoxy | Y¹ is N, Y² is CH, |
| | fluoro | | | Y³ is CH |
| P.0945 | X¹ is fluoro, X² is C—F, X³ is | chlorodifluoromethyl | methyl | Y¹ is N, Y² is CH, |
| | fluoro | | | Y³ is CH |
| P.0946 | X¹ is fluoro, X² is C—F, X³ is | chlorodifluoromethyl | nitro | Y¹ is N, Y² is CH, |
| | fluoro | | | Y³ is CH |
| P.0947 | X¹ is fluoro, X² is C—F, X³ is | chlorodifluoromethyl | trifluoromethoxy | Y¹ is N, Y² is CH, |
| | fluoro | | | Y³ is CH |
| P.0948 | X¹ is fluoro, X² is C—F, X³ is | chlorodifluoromethyl | trifluoromethyl | Y¹ is N, Y² is CH, |
| | fluoro | | | Y³ is CH |
| P.0949 | X¹ is fluoro, X² is C—F, X³ is | difluoromethyl | bromo | Y¹ is N, Y² is CH, |
| | fluoro | | | Y³ is CH |
| P.0950 | X¹ is fluoro, X² is C—F, X³ is | difluoromethyl | chloro | Y¹ is N, Y² is CH, |
| | fluoro | | | Y³ is CH |
| P.0951 | X¹ is fluoro, X² is C—F, X³ is | difluoromethyl | cyano | Y¹ is N, Y² is CH, |
| | fluoro | | | Y³ is CH |
| P.0952 | X¹ is fluoro, X² is C—F, X³ is | difluoromethyl | cyclopropyl | Y¹ is N, Y² is CH, |
| | fluoro | | | Y³ is CH |
| P.0953 | X¹ is fluoro, X² is C—F, X³ is | difluoromethyl | ethyl | Y¹ is N, Y² is CH, |
| | fluoro | | | Y³ is CH |
| P.0954 | X¹ is fluoro, X² is C—F, X³ is | difluoromethyl | fluoro | Y¹ is N, Y² is CH, |
| | fluoro | | | Y³ is CH |
| P.0955 | X¹ is fluoro, X² is C—F, X³ is | difluoromethyl | hydrogen | Y¹ is N, Y² is CH, |
| | fluoro | | | Y³ is CH |
| P.0956 | X¹ is fluoro, X² is C—F, X³ is | difluoromethyl | methoxy | Y¹ is N, Y² is CH, |
| | fluoro | | | Y³ is CH |
| P.0957 | X¹ is fluoro, X² is C—F, X³ is | difluoromethyl | methyl | Y¹ is N, Y² is CH, |
| | fluoro | | | Y³ is CH |
| P.0958 | X¹ is fluoro, X² is C—F, X³ is | difluoromethyl | nitro | Y¹ is N, Y² is CH, |
| | fluoro | | | Y³ is CH |
| P.0959 | X¹ is fluoro, X² is C—F, X³ is | difluoromethyl | trifluoromethoxy | Y¹ is N, Y² is CH, |
| | fluoro | | | Y³ is CH |
| P.0960 | X¹ is fluoro, X² is C—F, X³ is | difluoromethyl | trifluoromethyl | Y¹ is N, Y² is CH, |
| | fluoro | | | Y³ is CH |
| P.0961 | X¹ is fluoro, X² is C—F, X³ is | trifluoromethyl | bromo | Y¹ is N, Y² is CH, |
| | fluoro | | | Y³ is CH |
| P.0962 | X¹ is fluoro, X² is C—F, X³ is | trifluoromethyl | chloro | Y¹ is N, Y² is CH, |
| | fluoro | | | Y³ is CH |
| P.0963 | X¹ is fluoro, X² is C—F, X³ is | trifluoromethyl | cyano | Y¹ is N, Y² is CH, |
| | fluoro | | | Y³ is CH |
| P.0964 | X¹ is fluoro, X² is C—F, X³ is | trifluoromethyl | cyclopropyl | Y¹ is N, Y² is CH, |
| | fluoro | | | Y³ is CH |
| P.0965 | X¹ is fluoro, X² is C—F, X³ is | trifluoromethyl | ethyl | Y¹ is N, Y² is CH, |
| | fluoro | | | Y³ is CH |
| P.0966 | X¹ is fluoro, X² is C—F, X³ is | trifluoromethyl | fluoro | Y¹ is N, Y² is CH, |
| | fluoro | | | Y³ is CH |
| P.0967 | X¹ is fluoro, X² is C—F, X³ is | trifluoromethyl | hydrogen | Y¹ is N, Y² is CH, |
| | fluoro | | | Y³ is CH |
| P.0968 | X¹ is fluoro, X² is C—F, X³ is | trifluoromethyl | methoxy | Y¹ is N, Y² is CH, |
| | fluoro | | | Y³ is CH |
| P.0969 | X¹ is fluoro, X² is C—F, X³ is | trifluoromethyl | methyl | Y¹ is N, Y² is CH, |
| | fluoro | | | Y³ is CH |
| P.0970 | X¹ is fluoro, X² is C—F, X³ is | trifluoromethyl | nitro | Y¹ is N, Y² is CH, |
| | fluoro | | | Y³ is CH |
| P.0971 | X¹ is fluoro, X² is C—F, X³ is | trifluoromethyl | trifluoromethoxy | Y¹ is N, Y² is CH, |
| | fluoro | | | Y³ is CH |
| P.0972 | X¹ is fluoro, X² is C—F, X³ is | trifluoromethyl | trifluoromethyl | Y¹ is N, Y² is CH, |
| | fluoro | | | Y³ is CH |
| P.0973 | X¹ is chloro, X² is CH, X³ is | chlorodifluoromethyl | bromo | Y¹ is N, Y² is CH, |
| | bromo | | | Y³ is CH |
| P.0974 | X¹ is chloro, X² is CH, X³ is | chlorodifluoromethyl | chloro | Y¹ is N, Y² is CH, |
| | bromo | | | Y³ is CH |
| P.0975 | X¹ is chloro, X² is CH, X³ is | chlorodifluoromethyl | cyano | Y¹ is N, Y² is CH, |
| | bromo | | | Y³ is CH |
| P.0976 | X¹ is chloro, X² is CH, X³ is | chlorodifluoromethyl | cyclopropyl | Y¹ is N, Y² is CH, |
| | bromo | | | Y³ is CH |
| P.0977 | X¹ is chloro, X² is CH, X³ is | chlorodifluoromethyl | ethyl | Y¹ is N, Y² is CH, |
| | bromo | | | Y³ is CH |
| P.0978 | X¹ is chloro, X² is CH, X³ is | chlorodifluoromethyl | fluoro | Y¹ is N, Y² is CH, |
| | bromo | | | Y³ is CH |
| P.0979 | X¹ is chloro, X² is CH, X³ is | chlorodifluoromethyl | hydrogen | Y¹ is N, Y² is CH, |
| | bromo | | | Y³ is CH |
| P.0980 | X¹ is chloro, X² is CH, X³ is | chlorodifluoromethyl | methoxy | Y¹ is N, Y² is CH, |
| | bromo | | | Y³ is CH |
| P.0981 | X¹ is chloro, X² is CH, X³ is | chlorodifluoromethyl | methyl | Y¹ is N, Y² is CH, |
| | bromo | | | Y³ is CH |
| P.0982 | X¹ is chloro, X² is CH, X³ is | chlorodifluoromethyl | nitro | Y¹ is N, Y² is CH, |
| | bromo | | | Y³ is CH |
| P.0983 | X¹ is chloro, X² is CH, X³ is | chlorodifluoromethyl | trifluoromethoxy | Y¹ is N, Y² is CH, |
| | bromo | | | Y³ is CH |
| P.0984 | X¹ is chloro, X² is CH, X³ is | chlorodifluoromethyl | trifluoromethyl | Y¹ is N, Y² is CH, |
| | bromo | | | Y³ is CH |
| P.0985 | X¹ is chloro, X² is CH, X³ is | difluoromethyl | bromo | Y¹ is N, Y² is CH, |
| | bromo | | | Y³ is CH |
| P.0986 | X¹ is chloro, X² is CH, X³ is | difluoromethyl | chloro | Y¹ is N, Y² is CH, |
| | bromo | | | Y³ is CH |
| P.0987 | X¹ is chloro, X² is CH, X³ is | difluoromethyl | cyano | Y¹ is N, Y² is CH, |
| | bromo | | | Y³ is CH |
| P.0988 | X¹ is chloro, X² is CH, X³ is | difluoromethyl | cyclopropyl | Y¹ is N, Y² is CH, |
| | bromo | | | Y³ is CH |
| P.0989 | X¹ is chloro, X² is CH, X³ is | difluoromethyl | ethyl | Y¹ is N, Y² is CH, |
| | bromo | | | Y³ is CH |
| P.0990 | X¹ is chloro, X² is CH, X³ is | difluoromethyl | fluoro | Y¹ is N, Y² is CH, |
| | bromo | | | Y³ is CH |
| P.0991 | X¹ is chloro, X² is CH, X³ is | difluoromethyl | hydrogen | Y¹ is N, Y² is CH, |
| | bromo | | | Y³ is CH |
| P.0992 | X¹ is chloro, X² is CH, X³ is | difluoromethyl | methoxy | Y¹ is N, Y² is CH, |
| | bromo | | | Y³ is CH |
| P.0993 | X¹ is chloro, X² is CH, X³ is | difluoromethyl | methyl | Y¹ is N, Y² is CH, |
| | bromo | | | Y³ is CH |
| P.0994 | X¹ is chloro, X² is CH, X³ is | difluoromethyl | nitro | Y¹ is N, Y² is CH, |
| | bromo | | | Y³ is CH |
| P.0995 | X¹ is chloro, X² is CH, X³ is | difluoromethyl | trifluoromethoxy | Y¹ is N, Y² is CH, |
| | bromo | | | Y³ is CH |
| P.0996 | X¹ is chloro, X² is CH, X³ is | difluoromethyl | trifluoromethyl | Y¹ is N, Y² is CH, |
| | bromo | | | Y³ is CH |
| P.0997 | X¹ is chloro, X² is CH, X³ is | trifluoromethyl | bromo | Y¹ is N, Y² is CH, |
| | bromo | | | Y³ is CH |
| P.0998 | X¹ is chloro, X² is CH, X³ is | trifluoromethyl | chloro | Y¹ is N, Y² is CH, |
| | bromo | | | Y³ is CH |
| P.0999 | X¹ is chloro, X² is CH, X³ is | trifluoromethyl | cyano | Y¹ is N, Y² is CH, |
| | bromo | | | Y³ is CH |
| P.1000 | X¹ is chloro, X² is CH, X³ is | trifluoromethyl | cyclopropyl | Y¹ is N, Y² is CH, |
| | bromo | | | Y³ is CH |
| P.1001 | X¹ is chloro, X² is CH, X³ is | trifluoromethyl | ethyl | Y¹ is N, Y² is CH, |
| | bromo | | | Y³ is CH |
| P.1002 | X¹ is chloro, X² is CH, X³ is | trifluoromethyl | fluoro | Y¹ is N, Y² is CH, |
| | bromo | | | Y³ is CH |
| P.1003 | X¹ is chloro, X² is CH, X³ is | trifluoromethyl | hydrogen | Y¹ is N, Y² is CH, |
| | bromo | | | Y³ is CH |
| P.1004 | X¹ is chloro, X² is CH, X³ is | trifluoromethyl | methoxy | Y¹ is N, Y² is CH, |
| | bromo | | | Y³ is CH |
| P.1005 | X¹ is chloro, X² is CH, X³ is | trifluoromethyl | methyl | Y¹ is N, Y² is CH, |
| | bromo | | | Y³ is CH |
| P.1006 | X¹ is chloro, X² is CH, X³ is | trifluoromethyl | nitro | Y¹ is N, Y² is CH, |
| | bromo | | | Y³ is CH |
| P.1007 | X¹ is chloro, X² is CH, X³ is | trifluoromethyl | trifluoromethoxy | Y¹ is N, Y² is CH, |
| | bromo | | | Y³ is CH |
| P.1008 | X¹ is chloro, X² is CH, X³ is | trifluoromethyl | trifluoromethyl | Y¹ is N, Y² is CH, |
| | bromo | | | Y³ is CH |
| P.1009 | X¹ is chloro, X² is CH, X³ is | chlorodifluoromethyl | bromo | Y¹ is N, Y² is CH, |
| | bromo | | | Y³ is CH |
| P.1010 | X¹ is chloro, X² is CH, X³ is | chlorodifluoromethyl | chloro | Y¹ is N, Y² is CH, |
| | bromo | | | Y³ is CH |
| P.1011 | X¹ is chloro, X² is CH, X³ is | chlorodifluoromethyl | cyano | Y¹ is N, Y² is CH, |
| | bromo | | | Y³ is CH |
| P.1012 | X¹ is chloro, X² is CH, X³ is | chlorodifluoromethyl | cyclopropyl | Y¹ is N, Y² is CH, |
| | bromo | | | Y³ is CH |
| P.1013 | X¹ is chloro, X² is CH, X³ is | chlorodifluoromethyl | ethyl | Y¹ is N, Y² is CH, |
| | bromo | | | Y³ is CH |
| P.1014 | X¹ is chloro, X² is CH, X³ is | chlorodifluoromethyl | fluoro | Y¹ is N, Y² is CH, |
| | bromo | | | Y³ is CH |
| P.1015 | X¹ is chloro, X² is CH, X³ is | chlorodifluoromethyl | hydrogen | Y¹ is N, Y² is CH, |
| | bromo | | | Y³ is CH |
| P.1016 | X¹ is chloro, X² is CH, X³ is | chlorodifluoromethyl | methoxy | Y¹ is N, Y² is CH, |
| | bromo | | | Y³ is CH |
| P.1017 | X¹ is chloro, X² is CH, X³ is | chlorodifluoromethyl | methyl | Y¹ is N, Y² is CH, |
| | bromo | | | Y³ is CH |
| P.1018 | X¹ is chloro, X² is CH, X³ is | chlorodifluoromethyl | nitro | Y¹ is N, Y² is CH, |
| | bromo | | | Y³ is CH |
| P.1019 | X¹ is chloro, X² is CH, X³ is | chlorodifluoromethyl | trifluoromethoxy | Y¹ is N, Y² is CH, |
| | bromo | | | Y³ is CH |
| P.1020 | X¹ is chloro, X² is CH, X³ is | chlorodifluoromethyl | trifluoromethyl | Y¹ is N, Y² is CH, |
| | bromo | | | Y³ is CH |
| P.1021 | X¹ is chloro, X² is CH, X³ is | difluoromethyl | bromo | Y¹ is N, Y² is CH, |
| | bromo | | | Y³ is CH |
| P.1022 | X¹ is chloro, X² is CH, X³ is | difluoromethyl | chloro | Y¹ is N, Y² is CH, |
| | bromo | | | Y³ is CH |
| P.1023 | X¹ is chloro, X² is CH, X³ is | difluoromethyl | cyano | Y¹ is N, Y² is CH, |
| | bromo | | | Y³ is CH |
| P.1024 | X¹ is chloro, X² is CH, X³ is | difluoromethyl | cyclopropyl | Y¹ is N, Y² is CH, |
| | bromo | | | Y³ is CH |
| P.1025 | X¹ is chloro, X² is CH, X³ is | difluoromethyl | ethyl | Y¹ is N, Y² is CH, |
| | bromo | | | Y³ is CH |
| P.1026 | X¹ is chloro, X² is CH, X³ is | difluoromethyl | fluoro | Y¹ is N, Y² is CH, |
| | bromo | | | Y³ is CH |
| P.1027 | X¹ is chloro, X² is CH, X³ is | difluoromethyl | hydrogen | Y¹ is N, Y² is CH, |
| | bromo | | | Y³ is CH |
| P.1028 | X¹ is chloro, X² is CH, X³ is | difluoromethyl | methoxy | Y¹ is N, Y² is CH, |
| | bromo | | | Y³ is CH |
| P.1029 | X¹ is chloro, X² is CH, X³ is | difluoromethyl | methyl | Y¹ is N, Y² is CH, |
| | bromo | | | Y³ is CH |
| P.1030 | X¹ is chloro, X² is CH, X³ is | difluoromethyl | nitro | Y¹ is N, Y² is CH, |
| | bromo | | | Y³ is CH |
| P.1031 | X¹ is chloro, X² is CH, X³ is | difluoromethyl | trifluoromethoxy | Y¹ is N, Y² is CH, |
| | bromo | | | Y³ is CH |
| P.1032 | X¹ is chloro, X² is CH, X³ is | difluoromethyl | trifluoromethyl | Y¹ is N, Y² is CH, |
| | bromo | | | Y³ is CH |
| P.1033 | X¹ is chloro, X² is CH, X³ is | trifluoromethyl | bromo | Y¹ is N, Y² is CH, |
| | bromo | | | Y³ is CH |
| P.1034 | X¹ is chloro, X² is CH, X³ is | trifluoromethyl | chloro | Y¹ is N, Y² is CH, |
| | bromo | | | Y³ is CH |
| P.1035 | X¹ is chloro, X² is CH, X³ is | trifluoromethyl | cyano | Y¹ is N, Y² is CH, |
| | bromo | | | Y³ is CH |
| P.1036 | X¹ is chloro, X² is CH, X³ is | trifluoromethyl | cyclopropyl | Y¹ is N, Y² is CH, |
| | bromo | | | Y³ is CH |
| P.1037 | X¹ is chloro, X² is CH, X³ is | trifluoromethyl | ethyl | Y¹ is N, Y² is CH, |
| | bromo | | | Y³ is CH |
| P.1038 | X¹ is chloro, X² is CH, X³ is | trifluoromethyl | fluoro | Y¹ is N, Y² is CH, |
| | bromo | | | Y³ is CH |
| P.1039 | X¹ is chloro, X² is CH, X³ is | trifluoromethyl | hydrogen | Y¹ is N, Y² is CH, |
| | bromo | | | Y³ is CH |
| P.1040 | X¹ is chloro, X² is CH, X³ is | trifluoromethyl | methoxy | Y¹ is N, Y² is CH, |
| | bromo | | | Y³ is CH |
| P.1041 | X¹ is chloro, X² is CH, X³ is | trifluoromethyl | methyl | Y¹ is N, Y² is CH, |
| | bromo | | | Y³ is CH |
| P.1042 | X¹ is chloro, X² is CH, X³ is | trifluoromethyl | nitro | Y¹ is N, Y² is CH, |
| | bromo | | | Y³ is CH |
| P.1043 | X¹ is chloro, X² is CH, X³ is | trifluoromethyl | trifluoromethoxy | Y¹ is N, Y² is CH, |
| | bromo | | | Y³ is CH |
| P.1044 | X¹ is chloro, X² is CH, X³ is | trifluoromethyl | trifluoromethyl | Y¹ is N, Y² is CH, |
| | bromo | | | Y³ is CH |
| P.1045 | X¹ is chloro, X² is CH, X³ is | chlorodifluoromethyl | bromo | Y¹ is N, Y² is CH, |
| | bromo | | | Y³ is CH |
| P.1046 | X¹ is chloro, X² is CH, X³ is | chlorodifluoromethyl | chloro | Y¹ is N, Y² is CH, |
| | bromo | | | Y³ is CH |
| P.1047 | X¹ is chloro, X² is CH, X³ is | chlorodifluoromethyl | cyano | Y¹ is N, Y² is CH, |
| | bromo | | | Y³ is CH |
| P.1048 | X¹ is chloro, X² is CH, X³ is | chlorodifluoromethyl | cyclopropyl | Y¹ is N, Y² is CH, |
| | bromo | | | Y³ is CH |
| P.1049 | X¹ is chloro, X² is CH, X³ is | chlorodifluoromethyl | ethyl | Y¹ is N, Y² is CH, |
| | bromo | | | Y³ is CH |
| P.1050 | X¹ is chloro, X² is CH, X³ is | chlorodifluoromethyl | fluoro | Y¹ is N, Y² is CH, |
| | bromo | | | Y³ is CH |
| P.1051 | X¹ is chloro, X² is CH, X³ is | chlorodifluoromethyl | hydrogen | Y¹ is N, Y² is CH, |
| | bromo | | | Y³ is CH |
| P.1052 | X¹ is chloro, X² is CH, X³ is | chlorodifluoromethyl | methoxy | Y¹ is N, Y² is CH, |
| | bromo | | | Y³ is CH |
| P.1053 | X¹ is chloro, X² is CH, X³ is | chlorodifluoromethyl | methyl | Y¹ is N, Y² is CH, |
| | bromo | | | Y³ is CH |
| P.1054 | X¹ is chloro, X² is CH, X³ is | chlorodifluoromethyl | nitro | Y¹ is N, Y² is CH, |
| | bromo | | | Y³ is CH |
| P.1055 | X¹ is chloro, X² is CH, X³ is | chlorodifluoromethyl | trifluoromethoxy | Y¹ is N, Y² is CH, |
| | bromo | | | Y³ is CH |
| P.1056 | X¹ is chloro, X² is CH, X³ is | chlorodifluoromethyl | trifluoromethyl | Y¹ is N, Y² is CH, |
| | bromo | | | Y³ is CH |
| P.1057 | X¹ is chloro, X² is CH, X³ is | difluoromethyl | bromo | Y¹ is N, Y² is CH, |
| | bromo | | | Y³ is CH |
| P.1058 | X¹ is chloro, X² is CH, X³ is | difluoromethyl | chloro | Y¹ is N, Y² is CH, |
| | bromo | | | Y³ is CH |
| P.1059 | X¹ is chloro, X² is CH, X³ is | difluoromethyl | cyano | Y¹ is N, Y² is CH, |
| | bromo | | | Y³ is CH |
| P.1060 | X¹ is chloro, X² is CH, X³ is | difluoromethyl | cyclopropyl | Y¹ is N, Y² is CH, |
| | bromo | | | Y³ is CH |
| P.1061 | X¹ is chloro, X² is CH, X³ is | difluoromethyl | ethyl | Y¹ is N, Y² is CH, |
| | bromo | | | Y³ is CH |
| P.1062 | X¹ is chloro, X² is CH, X³ is | difluoromethyl | fluoro | Y¹ is N, Y² is CH, |
| | bromo | | | Y³ is CH |
| P.1063 | X¹ is chloro, X² is CH, X³ is | difluoromethyl | hydrogen | Y¹ is N, Y² is CH, |
| | bromo | | | Y³ is CH |
| P.1064 | X¹ is chloro, X² is CH, X³ is | difluoromethyl | methoxy | Y¹ is N, Y² is CH, |
| | bromo | | | Y³ is CH |
| P.1065 | X¹ is chloro, X² is CH, X³ is | difluoromethyl | methyl | Y¹ is N, Y² is CH, |
| | bromo | | | Y³ is CH |
| P.1066 | X¹ is chloro, X² is CH, X³ is | difluoromethyl | nitro | Y¹ is N, Y² is CH, |
| | bromo | | | Y³ is CH |
| P.1067 | X¹ is chloro, X² is CH, X³ is | difluoromethyl | trifluoromethoxy | Y¹ is N, Y² is CH, |
| | bromo | | | Y³ is CH |
| P.1068 | X¹ is chloro, X² is CH, X³ is | difluoromethyl | trifluoromethyl | Y¹ is N, Y² is CH, |
| | bromo | | | Y³ is CH |
| P.1069 | X¹ is chloro, X² is CH, X³ is | trifluoromethyl | bromo | Y¹ is N, Y² is CH, |
| | bromo | | | Y³ is CH |
| P.1070 | X¹ is chloro, X² is CH, X³ is | trifluoromethyl | chloro | Y¹ is N, Y² is CH, |
| | bromo | | | Y³ is CH |
| P.1071 | X¹ is chloro, X² is CH, X³ is | trifluoromethyl | cyano | Y¹ is N, Y² is CH, |
| | bromo | | | Y³ is CH |
| P.1072 | X¹ is chloro, X² is CH, X³ is | trifluoromethyl | cyclopropyl | Y¹ is N, Y² is CH, |
| | bromo | | | Y³ is CH |
| P.1073 | X¹ is chloro, X² is CH, X³ is | trifluoromethyl | ethyl | Y¹ is N, Y² is CH, |
| | bromo | | | Y³ is CH |
| P.1074 | X¹ is chloro, X² is CH, X³ is | trifluoromethyl | fluoro | Y¹ is N, Y² is CH, |
| | bromo | | | Y³ is CH |
| P.1075 | X¹ is chloro, X² is CH, X³ is | trifluoromethyl | hydrogen | Y¹ is N, Y² is CH, |
| | bromo | | | Y³ is CH |
| P.1076 | X¹ is chloro, X² is CH, X³ is | trifluoromethyl | methoxy | Y¹ is N, Y² is CH, |
| | bromo | | | Y³ is CH |
| P.1077 | X¹ is chloro, X² is CH, X³ is | trifluoromethyl | methyl | Y¹ is N, Y² is CH, |
| | bromo | | | Y³ is CH |
| P.1078 | X¹ is chloro, X² is CH, X³ is | trifluoromethyl | nitro | Y¹ is N, Y² is CH, |
| | bromo | | | Y³ is CH |
| P.1079 | X¹ is chloro, X² is CH, X³ is | trifluoromethyl | trifluoromethoxy | Y¹ is N, Y² is CH, |
| | bromo | | | Y³ is CH |
| P.1080 | X¹ is chloro, X² is CH, X³ is | trifluoromethyl | trifluoromethyl | Y¹ is N, Y² is CH, |
| | bromo | | | Y³ is CH |
| P.1081 | X¹ is chloro, X² is C—Cl, X³ | chlorodifluoromethyl | bromo | Y¹ is N, Y² is CH, |
| | is trifluoromethyl | | | Y³ is CH |
| P.1082 | X¹ is chloro, X² is C—Cl, X³ | chlorodifluoromethyl | chloro | Y¹ is N, Y² is CH, |
| | is trifluoromethyl | | | Y³ is CH |
| P.1083 | X¹ is chloro, X² is C—Cl, X³ | chlorodifluoromethyl | cyano | Y¹ is N, Y² is CH, |
| | is trifluoromethyl | | | Y³ is CH |
| P.1084 | X¹ is chloro, X² is C—Cl, X³ | chlorodifluoromethyl | cyclopropyl | Y¹ is N, Y² is CH, |
| | is trifluoromethyl | | | Y³ is CH |
| P.1085 | X¹ is chloro, X² is C—Cl, X³ | chlorodifluoromethyl | ethyl | Y¹ is N, Y² is CH, |
| | is trifluoromethyl | | | Y³ is CH |
| P.1086 | X¹ is chloro, X² is C—Cl, X³ | chlorodifluoromethyl | fluoro | Y¹ is N, Y² is CH, |
| | is trifluoromethyl | | | Y³ is CH |
| P.1087 | X¹ is chloro, X² is C—Cl, X³ | chlorodifluoromethyl | hydrogen | Y¹ is N, Y² is CH, |
| | is trifluoromethyl | | | Y³ is CH |
| P.1088 | X¹ is chloro, X² is C—Cl, X³ | chlorodifluoromethyl | methoxy | Y¹ is N, Y² is CH, |
| | is trifluoromethyl | | | Y³ is CH |
| P.1089 | X¹ is chloro, X² is C—Cl, X³ | chlorodifluoromethyl | methyl | Y¹ is N, Y² is CH, |
| | is trifluoromethyl | | | Y³ is CH |
| P.1090 | X¹ is chloro, X² is C—Cl, X³ | chlorodifluoromethyl | nitro | Y¹ is N, Y² is CH, |
| | is trifluoromethyl | | | Y³ is CH |
| P.1091 | X¹ is chloro, X² is C—Cl, X³ | chlorodifluoromethyl | trifluoromethoxy | Y¹ is N, Y² is CH, |
| | is trifluoromethyl | | | Y³ is CH |
| P.1092 | X¹ is chloro, X² is C—Cl, X³ | chlorodifluoromethyl | trifluoromethyl | Y¹ is N, Y² is CH, |
| | is trifluoromethyl | | | Y³ is CH |
| P.1093 | X¹ is chloro, X² is C—Cl, X³ | difluoromethyl | bromo | Y¹ is N, Y² is CH, |
| | is trifluoromethyl | | | Y³ is CH |
| P.1094 | X¹ is chloro, X² is C—Cl, X³ | difluoromethyl | chloro | Y¹ is N, Y² is CH, |
| | is trifluoromethyl | | | Y³ is CH |
| P.1095 | X¹ is chloro, X² is C—Cl, X³ | difluoromethyl | cyano | Y¹ is N, Y² is CH, |
| | is trifluoromethyl | | | Y³ is CH |
| P.1096 | X¹ is chloro, X² is C—Cl, X³ | difluoromethyl | cyclopropyl | Y¹ is N, Y² is CH, |
| | is trifluoromethyl | | | Y³ is CH |
| P.1097 | X¹ is chloro, X² is C—Cl, X³ | difluoromethyl | ethyl | Y¹ is N, Y² is CH, |
| | is trifluoromethyl | | | Y³ is CH |
| P.1098 | X¹ is chloro, X² is C—Cl, X³ | difluoromethyl | fluoro | Y¹ is N, Y² is CH, |
| | is trifluoromethyl | | | Y³ is CH |
| P.1099 | X¹ is chloro, X² is C—Cl, X³ | difluoromethyl | hydrogen | Y¹ is N, Y² is CH, |
| | is trifluoromethyl | | | Y³ is CH |
| P.1100 | X¹ is chloro, X² is C—Cl, X³ | difluoromethyl | methoxy | Y¹ is N, Y² is CH, |
| | is trifluoromethyl | | | Y³ is CH |
| P.1101 | X¹ is chloro, X² is C—Cl, X³ | difluoromethyl | methyl | Y¹ is N, Y² is CH, |
| | is trifluoromethyl | | | Y³ is CH |
| P.1102 | X¹ is chloro, X² is C—Cl, X³ | difluoromethyl | nitro | Y¹ is N, Y² is CH, |
| | is trifluoromethyl | | | Y³ is CH |
| P.1103 | X¹ is chloro, X² is C—Cl, X³ | difluoromethyl | trifluoromethoxy | Y¹ is N, Y² is CH, |
| | is trifluoromethyl | | | Y³ is CH |
| P.1104 | X¹ is chloro, X² is C—Cl, X³ | difluoromethyl | trifluoromethyl | Y¹ is N, Y² is CH, |
| | is trifluoromethyl | | | Y³ is CH |
| P.1105 | X¹ is chloro, X² is C—Cl, X³ | trifluoromethyl | bromo | Y¹ is N, Y² is CH, |
| | is trifluoromethyl | | | Y³ is CH |
| P.1106 | X¹ is chloro, X² is C—Cl, X³ | trifluoromethyl | chloro | Y¹ is N, Y² is CH, |
| | is trifluoromethyl | | | Y³ is CH |
| P.1107 | X¹ is chloro, X² is C—Cl, X³ | trifluoromethyl | cyano | Y¹ is N, Y² is CH, |
| | is trifluoromethyl | | | Y³ is CH |
| P.1108 | X¹ is chloro, X² is C—Cl, X³ | trifluoromethyl | cyclopropyl | Y¹ is N, Y² is CH, |
| | is trifluoromethyl | | | Y³ is CH |
| P.1109 | X¹ is chloro, X² is C—Cl, X³ | trifluoromethyl | ethyl | Y¹ is N, Y² is CH, |
| | is trifluoromethyl | | | Y³ is CH |
| P.1110 | X¹ is chloro, X² is C—Cl, X³ | trifluoromethyl | fluoro | Y¹ is N, Y² is CH, |
| | is trifluoromethyl | | | Y³ is CH |
| P.1111 | X¹ is chloro, X² is C—Cl, X³ | trifluoromethyl | hydrogen | Y¹ is N, Y² is CH, |
| | is trifluoromethyl | | | Y³ is CH |
| P.1112 | X¹ is chloro, X² is C—Cl, X³ | trifluoromethyl | methoxy | Y¹ is N, Y² is CH, |
| | is trifluoromethyl | | | Y³ is CH |
| P.1113 | X¹ is chloro, X² is C—Cl, X³ | trifluoromethyl | methyl | Y¹ is N, Y² is CH, |
| | is trifluoromethyl | | | Y³ is CH |
| P.1114 | X¹ is chloro, X² is C—Cl, X³ | trifluoromethyl | nitro | Y¹ is N, Y² is CH, |
| | is trifluoromethyl | | | Y³ is CH |
| P.1115 | X¹ is chloro, X² is C—Cl, X³ | trifluoromethyl | trifluoromethoxy | Y¹ is N, Y² is CH, |
| | is trifluoromethyl | | | Y³ is CH |
| P.1116 | X¹ is chloro, X² is C—Cl, X³ | trifluoromethyl | trifluoromethyl | Y¹ is N, Y² is CH, |
| | is trifluoromethyl | | | Y³ is CH |
| P.1117 | X¹ is trifluoromethyl, X² is | chlorodifluoromethyl | bromo | Y¹ is N, Y² is CH, |
| | CH, X³ is trifluoromethyl | | | Y³ is CH |
| P.1118 | X¹ is trifluoromethyl, X² is | chlorodifluoromethyl | chloro | Y¹ is N, Y² is CH, |
| | CH, X³ is trifluoromethyl | | | Y³ is CH |
| P.1119 | X¹ is trifluoromethyl, X² is | chlorodifluoromethyl | cyano | Y¹ is N, Y² is CH, |
| | CH, X³ is trifluoromethyl | | | Y³ is CH |
| P.1120 | X¹ is trifluoromethyl, X² is | chlorodifluoromethyl | cyclopropyl | Y¹ is N, Y² is CH, |
| | CH, X³ is trifluoromethyl | | | Y³ is CH |
| P.1121 | X¹ is trifluoromethyl, X² is | chlorodifluoromethyl | ethyl | Y¹ is N, Y² is CH, |
| | CH, X³ is trifluoromethyl | | | Y³ is CH |
| P.1122 | X¹ is trifluoromethyl, X² is | chlorodifluoromethyl | fluoro | Y¹ is N, Y² is CH, |
| | CH, X³ is trifluoromethyl | | | Y³ is CH |
| P.1123 | X¹ is trifluoromethyl, X² is | chlorodifluoromethyl | hydrogen | Y¹ is N, Y² is CH, |
| | CH, X³ is trifluoromethyl | | | Y³ is CH |
| P.1124 | X¹ is trifluoromethyl, X² is | chlorodifluoromethyl | methoxy | Y¹ is N, Y² is CH, |
| | CH, X³ is trifluoromethyl | | | Y³ is CH |
| P.1125 | X¹ is trifluoromethyl, X² is | chlorodifluoromethyl | methyl | Y¹ is N, Y² is CH, |
| | CH, X³ is trifluoromethyl | | | Y³ is CH |
| P.1126 | X¹ is trifluoromethyl, X² is | chlorodifluoromethyl | nitro | Y¹ is N, Y² is CH, |
| | CH, X³ is trifluoromethyl | | | Y³ is CH |
| P.1127 | X¹ is trifluoromethyl, X² is | chlorodifluoromethyl | trifluoromethoxy | Y¹ is N, Y² is CH, |
| | CH, X³ is trifluoromethyl | | | Y³ is CH |
| P.1128 | X¹ is trifluoromethyl, X² is | chlorodifluoromethyl | trifluoromethyl | Y¹ is N, Y² is CH, |
| | CH, X³ is trifluoromethyl | | | Y³ is CH |
| P.1129 | X¹ is trifluoromethyl, X² is | difluoromethyl | bromo | Y¹ is N, Y² is CH, |
| | CH, X³ is trifluoromethyl | | | Y³ is CH |
| P.1130 | X¹ is trifluoromethyl, X² is | difluoromethyl | chloro | Y¹ is N, Y² is CH, |
| | CH, X³ is trifluoromethyl | | | Y³ is CH |
| P.1131 | X¹ is trifluoromethyl, X² is | difluoromethyl | cyano | Y¹ is N, Y² is CH, |
| | CH, X³ is trifluoromethyl | | | Y³ is CH |
| P.1132 | X¹ is trifluoromethyl, X² is | difluoromethyl | cyclopropyl | Y¹ is N, Y² is CH, |
| | CH, X³ is trifluoromethyl | | | Y³ is CH |
| P.1133 | X¹ is trifluoromethyl, X² is | difluoromethyl | ethyl | Y¹ is N, Y² is CH, |
| | CH, X³ is trifluoromethyl | | | Y³ is CH |
| P.1134 | X¹ is trifluoromethyl, X² is | difluoromethyl | fluoro | Y¹ is N, Y² is CH, |
| | CH, X³ is trifluoromethyl | | | Y³ is CH |
| P.1135 | X¹ is trifluoromethyl, X² is | difluoromethyl | hydrogen | Y¹ is N, Y² is CH, |
| | CH, X³ is trifluoromethyl | | | Y³ is CH |
| P.1136 | X¹ is trifluoromethyl, X² is | difluoromethyl | methoxy | Y¹ is N, Y² is CH, |
| | CH, X³ is trifluoromethyl | | | Y³ is CH |
| P.1137 | X¹ is trifluoromethyl, X² is | difluoromethyl | methyl | Y¹ is N, Y² is CH, |
| | CH, X³ is trifluoromethyl | | | Y³ is CH |
| P.1138 | X¹ is trifluoromethyl, X² is | difluoromethyl | nitro | Y¹ is N, Y² is CH, |
| | CH, X³ is trifluoromethyl | | | Y³ is CH |
| P.1139 | X¹ is trifluoromethyl, X² is | difluoromethyl | trifluoromethoxy | Y¹ is N, Y² is CH, |
| | CH, X³ is trifluoromethyl | | | Y³ is CH |
| P.1140 | X¹ is trifluoromethyl, X² is | difluoromethyl | trifluoromethyl | Y¹ is N, Y² is CH, |
| | CH, X³ is trifluoromethyl | | | Y³ is CH |
| P.1141 | X¹ is trifluoromethyl, X² is | trifluoromethyl | bromo | Y¹ is N, Y² is CH, |
| | CH, X³ is trifluoromethyl | | | Y³ is CH |
| P.1142 | X¹ is trifluoromethyl, X² is | trifluoromethyl | chloro | Y¹ is N, Y² is CH, |
| | CH, X³ is trifluoromethyl | | | Y³ is CH |
| P.1143 | X¹ is trifluoromethyl, X² is | trifluoromethyl | cyano | Y¹ is N, Y² is CH, |
| | CH, X³ is trifluoromethyl | | | Y³ is CH |
| P.1144 | X¹ is trifluoromethyl, X² is | trifluoromethyl | cyclopropyl | Y¹ is N, Y² is CH, |
| | CH, X³ is trifluoromethyl | | | Y³ is CH |
| P.1145 | X¹ is trifluoromethyl, X² is | trifluoromethyl | ethyl | Y¹ is N, Y² is CH, |
| | CH, X³ is trifluoromethyl | | | Y³ is CH |
| P.1146 | X¹ is trifluoromethyl, X² is | trifluoromethyl | fluoro | Y¹ is N, Y² is CH, |
| | CH, X³ is trifluoromethyl | | | Y³ is CH |
| P.1147 | X¹ is trifluoromethyl, X² is | trifluoromethyl | hydrogen | Y¹ is N, Y² is CH, |
| | CH, X³ is trifluoromethyl | | | Y³ is CH |
| P.1148 | X¹ is trifluoromethyl, X² is | trifluoromethyl | methoxy | Y¹ is N, Y² is CH, |
| | CH, X³ is trifluoromethyl | | | Y³ is CH |
| P.1149 | X¹ is trifluoromethyl, X² is | trifluoromethyl | methyl | Y¹ is N, Y² is CH, |
| | CH, X³ is trifluoromethyl | | | Y³ is CH |
| P.1150 | X¹ is trifluoromethyl, X² is | trifluoromethyl | nitro | Y¹ is N, Y² is CH, |
| | CH, X³ is trifluoromethyl | | | Y³ is CH |
| P.1151 | X¹ is trifluoromethyl, X² is | trifluoromethyl | trifluoromethoxy | Y¹ is N, Y² is CH, |
| | CH, X³ is trifluoromethyl | | | Y³ is CH |
| P.1152 | X¹ is trifluoromethyl, X² is | trifluoromethyl | trifluoromethyl | Y¹ is N, Y² is CH, |
| | CH, X³ is trifluoromethyl | | | Y³ is CH |
| P.1153 | X¹ is trifluoromethyl, X² is | chlorodifluoromethyl | bromo | Y¹ is N, Y² is CH, |
| | CH, X³ is trifluoromethyl | | | Y³ is CH |
| P.1154 | X¹ is trifluoromethyl, X² is | chlorodifluoromethyl | chloro | Y¹ is N, Y² is CH, |
| | CH, X³ is trifluoromethyl | | | Y³ is CH |
| P.1155 | X¹ is trifluoromethyl, X² is | chlorodifluoromethyl | cyano | Y¹ is N, Y² is CH, |
| | CH, X³ is trifluoromethyl | | | Y³ is CH |
| P.1156 | X¹ is trifluoromethyl, X² is | chlorodifluoromethyl | cyclopropyl | Y¹ is N, Y² is CH, |
| | CH, X³ is trifluoromethyl | | | Y³ is CH |
| P.1157 | X¹ is trifluoromethyl, X² is | chlorodifluoromethyl | ethyl | Y¹ is N, Y² is CH, |
| | CH, X³ is trifluoromethyl | | | Y³ is CH |
| P.1158 | X¹ is trifluoromethyl, X² is | chlorodifluoromethyl | fluoro | Y¹ is N, Y² is CH, |
| | CH, X³ is trifluoromethyl | | | Y³ is CH |
| P.1159 | X¹ is trifluoromethyl, X² is | chlorodifluoromethyl | hydrogen | Y¹ is N, Y² is CH, |
| | CH, X³ is trifluoromethyl | | | Y³ is CH |
| P.1160 | X¹ is trifluoromethyl, X² is | chlorodifluoromethyl | methoxy | Y¹ is N, Y² is CH, |
| | CH, X³ is trifluoromethyl | | | Y³ is CH |
| P.1161 | X¹ is trifluoromethyl, X² is | chlorodifluoromethyl | methyl | Y¹ is N, Y² is CH, |
| | CH, X³ is trifluoromethyl | | | Y³ is CH |
| P.1162 | X¹ is trifluoromethyl, X² is | chlorodifluoromethyl | nitro | Y¹ is N, Y² is CH, |
| | CH, X³ is trifluoromethyl | | | Y³ is CH |
| P.1163 | X¹ is trifluoromethyl, X² is | chlorodifluoromethyl | trifluoromethoxy | Y¹ is N, Y² is CH, |
| | CH, X³ is trifluoromethyl | | | Y³ is CH |
| P.1164 | X¹ is trifluoromethyl, X² is | chlorodifluoromethyl | trifluoromethyl | Y¹ is N, Y² is CH, |
| | CH, X³ is trifluoromethyl | | | Y³ is CH |
| P.1165 | X¹ is trifluoromethyl, X² is | difluoromethyl | bromo | Y¹ is N, Y² is CH, |
| | CH, X³ is trifluoromethyl | | | Y³ is CH |
| P.1166 | X¹ is trifluoromethyl, X² is | difluoromethyl | chloro | Y¹ is N, Y² is CH, |
| | CH, X³ is trifluoromethyl | | | Y³ is CH |
| P.1167 | X¹ is trifluoromethyl, X² is | difluoromethyl | cyano | Y¹ is N, Y² is CH, |
| | CH, X³ is trifluoromethyl | | | Y³ is CH |
| P.1168 | X¹ is trifluoromethyl, X² is | difluoromethyl | cyclopropyl | Y¹ is N, Y² is CH, |
| | CH, X³ is trifluoromethyl | | | Y³ is CH |
| P.1169 | X¹ is trifluoromethyl, X² is | difluoromethyl | ethyl | Y¹ is N, Y² is CH, |
| | CH, X³ is trifluoromethyl | | | Y³ is CH |
| P.1170 | X¹ is trifluoromethyl, X² is | difluoromethyl | fluoro | Y¹ is N, Y² is CH, |
| | CH, X³ is trifluoromethyl | | | Y³ is CH |
| P.1171 | X¹ is trifluoromethyl, X² is | difluoromethyl | hydrogen | Y¹ is N, Y² is CH, |
| | CH, X³ is trifluoromethyl | | | Y³ is CH |
| P.1172 | X¹ is trifluoromethyl, X² is | difluoromethyl | methoxy | Y¹ is N, Y² is CH, |
| | CH, X³ is trifluoromethyl | | | Y³ is CH |
| P.1173 | X¹ is trifluoromethyl, X² is | difluoromethyl | methyl | Y¹ is N, Y² is CH, |
| | CH, X³ is trifluoromethyl | | | Y³ is CH |
| P.1174 | X¹ is trifluoromethyl, X² is | difluoromethyl | nitro | Y¹ is N, Y² is CH, |
| | CH, X³ is trifluoromethyl | | | Y³ is CH |
| P.1175 | X¹ is trifluoromethyl, X² is | difluoromethyl | trifluoromethoxy | Y¹ is N, Y² is CH, |
| | CH, X³ is trifluoromethyl | | | Y³ is CH |
| P.1176 | X¹ is trifluoromethyl, X² is | difluoromethyl | trifluoromethyl | Y¹ is N, Y² is CH, |
| | CH, X³ is trifluoromethyl | | | Y³ is CH |
| P.1177 | X¹ is trifluoromethyl, X² is | trifluoromethyl | bromo | Y¹ is N, Y² is CH, |
| | CH, X³ is trifluoromethyl | | | Y³ is CH |
| P.1178 | X¹ is trifluoromethyl, X² is | trifluoromethyl | chloro | Y¹ is N, Y² is CH, |
| | CH, X³ is trifluoromethyl | | | Y³ is CH |
| P.1179 | X¹ is trifluoromethyl, X² is | trifluoromethyl | cyano | Y¹ is N, Y² is CH, |
| | CH, X³ is trifluoromethyl | | | Y³ is CH |
| P.1180 | X¹ is trifluoromethyl, X² is | trifluoromethyl | cyclopropyl | Y¹ is N, Y² is CH, |
| | CH, X³ is trifluoromethyl | | | Y³ is CH |
| P.1181 | X¹ is trifluoromethyl, X² is | trifluoromethyl | ethyl | Y¹ is N, Y² is CH, |
| | CH, X³ is trifluoromethyl | | | Y³ is CH |
| P.1182 | X¹ is trifluoromethyl, X² is | trifluoromethyl | fluoro | Y¹ is N, Y² is CH, |
| | CH, X³ is trifluoromethyl | | | Y³ is CH |
| P.1183 | X¹ is trifluoromethyl, X² is | trifluoromethyl | hydrogen | Y¹ is N, Y² is CH, |
| | CH, X³ is trifluoromethyl | | | Y³ is CH |
| P.1184 | X¹ is trifluoromethyl, X² is | trifluoromethyl | methoxy | Y¹ is N, Y² is CH, |
| | CH, X³ is trifluoromethyl | | | Y³ is CH |
| P.1185 | X¹ is trifluoromethyl, X² is | trifluoromethyl | methyl | Y¹ is N, Y² is CH, |
| | CH, X³ is trifluoromethyl | | | Y³ is CH |
| P.1186 | X¹ is trifluoromethyl, X² is | trifluoromethyl | nitro | Y¹ is N, Y² is CH, |
| | CH, X³ is trifluoromethyl | | | Y³ is CH |
| P.1187 | X¹ is trifluoromethyl, X² is | trifluoromethyl | trifluoromethoxy | Y¹ is N, Y² is CH, |
| | CH, X³ is trifluoromethyl | | | Y³ is CH |
| P.1188 | X¹ is trifluoromethyl, X² is | trifluoromethyl | trifluoromethyl | Y¹ is N, Y² is CH, |
| | CH, X³ is trifluoromethyl | | | Y³ is CH |
| P.1189 | X¹ is trifluoromethyl, X² is | chlorodifluoromethyl | bromo | Y¹ is N, Y² is CH, |
| | CH, X³ is hydrogen | | | Y³ is CH |
| P.1190 | X¹ is trifluoromethyl, X² is | chlorodifluoromethyl | chloro | Y¹ is N, Y² is CH, |
| | CH, X³ is hydrogen | | | Y³ is CH |
| P.1191 | X¹ is trifluoromethyl, X² is | chlorodifluoromethyl | cyano | Y¹ is N, Y² is CH, |
| | CH, X³ is hydrogen | | | Y³ is CH |
| P.1192 | X¹ is trifluoromethyl, X² is | chlorodifluoromethyl | cyclopropyl | Y¹ is N, Y² is CH, |
| | CH, X³ is hydrogen | | | Y³ is CH |
| P.1193 | X¹ is trifluoromethyl, X² is | chlorodifluoromethyl | ethyl | Y¹ is N, Y² is CH, |
| | CH, X³ is hydrogen | | | Y³ is CH |
| P.1194 | X¹ is trifluoromethyl, X² is | chlorodifluoromethyl | fluoro | Y¹ is N, Y² is CH, |
| | CH, X³ is hydrogen | | | Y³ is CH |
| P.1195 | X¹ is trifluoromethyl, X² is | chlorodifluoromethyl | hydrogen | Y¹ is N, Y² is CH, |
| | CH, X³ is hydrogen | | | Y³ is CH |
| P.1196 | X¹ is trifluoromethyl, X² is | chlorodifluoromethyl | methoxy | Y¹ is N, Y² is CH, |
| | CH, X³ is hydrogen | | | Y³ is CH |
| P.1197 | X¹ is trifluoromethyl, X² is | chlorodifluoromethyl | methyl | Y¹ is N, Y² is CH, |
| | CH, X³ is hydrogen | | | Y³ is CH |
| P.1198 | X¹ is trifluoromethyl, X² is | chlorodifluoromethyl | nitro | Y¹ is N, Y² is CH, |
| | CH, X³ is hydrogen | | | Y³ is CH |
| P.1199 | X¹ is trifluoromethyl, X² is | chlorodifluoromethyl | trifluoromethoxy | Y¹ is N, Y² is CH, |
| | CH, X³ is hydrogen | | | Y³ is CH |
| P.1200 | X¹ is trifluoromethyl, X² is | chlorodifluoromethyl | trifluoromethyl | Y¹ is N, Y² is CH, |
| | CH, X³ is hydrogen | | | Y³ is CH |
| P.1201 | X¹ is trifluoromethyl, X² is | difluoromethyl | bromo | Y¹ is N, Y² is CH, |
| | CH, X³ is hydrogen | | | Y³ is CH |
| P.1202 | X¹ is trifluoromethyl, X² is | difluoromethyl | chloro | Y¹ is N, Y² is CH, |
| | CH, X³ is hydrogen | | | Y³ is CH |
| P.1203 | X¹ is trifluoromethyl, X² is | difluoromethyl | cyano | Y¹ is N, Y² is CH, |
| | CH, X³ is hydrogen | | | Y³ is CH |
| P.1204 | X¹ is trifluoromethyl, X² is | difluoromethyl | cyclopropyl | Y¹ is N, Y² is CH, |
| | CH, X³ is hydrogen | | | Y³ is CH |
| P.1205 | X¹ is trifluoromethyl, X² is | difluoromethyl | ethyl | Y¹ is N, Y² is CH, |
| | CH, X³ is hydrogen | | | Y³ is CH |
| P.1206 | X¹ is trifluoromethyl, X² is | difluoromethyl | fluoro | Y¹ is N, Y² is CH, |
| | CH, X³ is hydrogen | | | Y³ is CH |
| P.1207 | X¹ is trifluoromethyl, X² is | difluoromethyl | hydrogen | Y¹ is N, Y² is CH, |
| | CH, X³ is hydrogen | | | Y³ is CH |
| P.1208 | X¹ is trifluoromethyl, X² is | difluoromethyl | methoxy | Y¹ is N, Y² is CH, |
| | CH, X³ is hydrogen | | | Y³ is CH |
| P.1209 | X¹ is trifluoromethyl, X² is | difluoromethyl | methyl | Y¹ is N, Y² is CH, |
| | CH, X³ is hydrogen | | | Y³ is CH |
| P.1210 | X¹ is trifluoromethyl, X² is | difluoromethyl | nitro | Y¹ is N, Y² is CH, |
| | CH, X³ is hydrogen | | | Y³ is CH |
| P.1211 | X¹ is trifluoromethyl, X² is | difluoromethyl | trifluoromethoxy | Y¹ is N, Y² is CH, |
| | CH, X³ is hydrogen | | | Y³ is CH |
| P.1212 | X¹ is trifluoromethyl, X² is | difluoromethyl | trifluoromethyl | Y¹ is N, Y² is CH, |
| | CH, X³ is hydrogen | | | Y³ is CH |
| P.1213 | X¹ is trifluoromethyl, X² is | trifluoromethyl | bromo | Y¹ is N, Y² is CH, |
| | CH, X³ is hydrogen | | | Y³ is CH |
| P.1214 | X¹ is trifluoromethyl, X² is | trifluoromethyl | chloro | Y¹ is N, Y² is CH, |
| | CH, X³ is hydrogen | | | Y³ is CH |
| P.1215 | X¹ is trifluoromethyl, X² is | trifluoromethyl | cyano | Y¹ is N, Y² is CH, |
| | CH, X³ is hydrogen | | | Y³ is CH |
| P.1216 | X¹ is trifluoromethyl, X² is | trifluoromethyl | cyclopropyl | Y¹ is N, Y² is CH, |
| | CH, X³ is hydrogen | | | Y³ is CH |
| P.1217 | X¹ is trifluoromethyl, X² is | trifluoromethyl | ethyl | Y¹ is N, Y² is CH, |
| | CH, X³ is hydrogen | | | Y³ is CH |
| P.1218 | X¹ is trifluoromethyl, X² is | trifluoromethyl | fluoro | Y¹ is N, Y² is CH, |
| | CH, X³ is hydrogen | | | Y³ is CH |
| P.1219 | X¹ is trifluoromethyl, X² is | trifluoromethyl | hydrogen | Y¹ is N, Y² is CH, |
| | CH, X³ is hydrogen | | | Y³ is CH |
| P.1220 | X¹ is trifluoromethyl, X² is | trifluoromethyl | methoxy | Y¹ is N, Y² is CH, |
| | CH, X³ is hydrogen | | | Y³ is CH |
| P.1221 | X¹ is trifluoromethyl, X² is | trifluoromethyl | methyl | Y¹ is N, Y² is CH, |
| | CH, X³ is hydrogen | | | Y³ is CH |
| P.1222 | X¹ is trifluoromethyl, X² is | trifluoromethyl | nitro | Y¹ is N, Y² is CH, |
| | CH, X³ is hydrogen | | | Y³ is CH |
| P.1223 | X¹ is trifluoromethyl, X² is | trifluoromethyl | trifluoromethoxy | Y¹ is N, Y² is CH, |
| | CH, X³ is hydrogen | | | Y³ is CH |
| P.1224 | X¹ is trifluoromethyl, X² is | trifluoromethyl | trifluoromethyl | Y¹ is N, Y² is CH, |
| | CH, X³ is hydrogen | | | Y³ is CH |
| P.1225 | X¹ is chloro, X² is N, X³ is | chlorodifluoromethyl | bromo | Y¹ is N, Y² is CH, |
| | chloro | | | Y³ is CH |
| P.1226 | X¹ is chloro, X² is N, X³ is | chlorodifluoromethyl | chloro | Y¹ is N, Y² is CH, |
| | chloro | | | Y³ is CH |
| P.1227 | X¹ is chloro, X² is N, X³ is | chlorodifluoromethyl | cyano | Y¹ is N, Y² is CH, |
| | chloro | | | Y³ is CH |
| P.1228 | X¹ is chloro, X² is N, X³ is | chlorodifluoromethyl | cyclopropyl | Y¹ is N, Y² is CH, |
| | chloro | | | Y³ is CH |
| P.1229 | X¹ is chloro, X² is N, X³ is | chlorodifluoromethyl | ethyl | Y¹ is N, Y² is CH, |
| | chloro | | | Y³ is CH |
| P.1230 | X¹ is chloro, X² is N, X³ is | chlorodifluoromethyl | fluoro | Y¹ is N, Y² is CH, |
| | chloro | | | Y³ is CH |
| P.1231 | X¹ is chloro, X² is N, X³ is | chlorodifluoromethyl | hydrogen | Y¹ is N, Y² is CH, |
| | chloro | | | Y³ is CH |
| P.1232 | X¹ is chloro, X² is N, X³ is | chlorodifluoromethyl | methoxy | Y¹ is N, Y² is CH, |
| | chloro | | | Y³ is CH |
| P.1233 | X¹ is chloro, X² is N, X³ is | chlorodifluoromethyl | methyl | Y¹ is N, Y² is CH, |
| | chloro | | | Y³ is CH |
| P.1234 | X¹ is chloro, X² is N, X³ is | chlorodifluoromethyl | nitro | Y¹ is N, Y² is CH, |
| | chloro | | | Y³ is CH |
| P.1235 | X¹ is chloro, X² is N, X³ is | chlorodifluoromethyl | trifluoromethoxy | Y¹ is N, Y² is CH, |
| | chloro | | | Y³ is CH |
| P.1236 | X¹ is chloro, X² is N, X³ is | chlorodifluoromethyl | trifluoromethyl | Y¹ is N, Y² is CH, |
| | chloro | | | Y³ is CH |
| P.1237 | X¹ is chloro, X² is N, X³ is | difluoromethyl | bromo | Y¹ is N, Y² is CH, |
| | chloro | | | Y³ is CH |
| P.1238 | X¹ is chloro, X² is N, X³ is | difluoromethyl | chloro | Y¹ is N, Y² is CH, |
| | chloro | | | Y³ is CH |
| P.1239 | X¹ is chloro, X² is N, X³ is | difluoromethyl | cyano | Y¹ is N, Y² is CH, |
| | chloro | | | Y³ is CH |
| P.1240 | X¹ is chloro, X² is N, X³ is | difluoromethyl | cyclopropyl | Y¹ is N, Y² is CH, |
| | chloro | | | Y³ is CH |
| P.1241 | X¹ is chloro, X² is N, X³ is | difluoromethyl | ethyl | Y¹ is N, Y² is CH, |
| | chloro | | | Y³ is CH |
| P.1242 | X¹ is chloro, X² is N, X³ is | difluoromethyl | fluoro | Y¹ is N, Y² is CH, |
| | chloro | | | Y³ is CH |
| P.1243 | X¹ is chloro, X² is N, X³ is | difluoromethyl | hydrogen | Y¹ is N, Y² is CH, |
| | chloro | | | Y³ is CH |
| P.1244 | X¹ is chloro, X² is N, X³ is | difluoromethyl | methoxy | Y¹ is N, Y² is CH, |
| | chloro | | | Y³ is CH |
| P.1245 | X¹ is chloro, X² is N, X³ is | difluoromethyl | methyl | Y¹ is N, Y² is CH, |
| | chloro | | | Y³ is CH |
| P.1246 | X¹ is chloro, X² is N, X³ is | difluoromethyl | nitro | Y¹ is N, Y² is CH, |
| | chloro | | | Y³ is CH |
| P.1247 | X¹ is chloro, X² is N, X³ is | difluoromethyl | trifluoromethoxy | Y¹ is N, Y² is CH, |
| | chloro | | | Y³ is CH |
| P.1248 | X¹ is chloro, X² is N, X³ is | difluoromethyl | trifluoromethyl | Y¹ is N, Y² is CH, |
| | chloro | | | Y³ is CH |
| P.1249 | X¹ is chloro, X² is N, X³ is | trifluoromethyl | bromo | Y¹ is N, Y² is CH, |
| | chloro | | | Y³ is CH |
| P.1250 | X¹ is chloro, X² is N, X³ is | trifluoromethyl | chloro | Y¹ is N, Y² is CH, |
| | chloro | | | Y³ is CH |
| P.1251 | X¹ is chloro, X² is N, X³ is | trifluoromethyl | cyano | Y¹ is N, Y² is CH, |
| | chloro | | | Y³ is CH |
| P.1252 | X¹ is chloro, X² is N, X³ is | trifluoromethyl | cyclopropyl | Y¹ is N, Y² is CH, |
| | chloro | | | Y³ is CH |
| P.1253 | X¹ is chloro, X² is N, X³ is | trifluoromethyl | ethyl | Y¹ is N, Y² is CH, |
| | chloro | | | Y³ is CH |
| P.1254 | X¹ is chloro, X² is N, X³ is | trifluoromethyl | fluoro | Y¹ is N, Y² is CH, |
| | chloro | | | Y³ is CH |
| P.1255 | X¹ is chloro, X² is N, X³ is | trifluoromethyl | hydrogen | Y¹ is N, Y² is CH, |
| | chloro | | | Y³ is CH |
| P.1256 | X¹ is chloro, X² is N, X³ is | trifluoromethyl | methoxy | Y¹ is N, Y² is CH, |
| | chloro | | | Y³ is CH |
| P.1257 | X¹ is chloro, X² is N, X³ is | trifluoromethyl | methyl | Y¹ is N, Y² is CH, |
| | chloro | | | Y³ is CH |
| P.1258 | X¹ is chloro, X² is N, X³ is | trifluoromethyl | nitro | Y¹ is N, Y² is CH, |
| | chloro | | | Y³ is CH |
| P.1259 | X¹ is chloro, X² is N, X³ is | trifluoromethyl | trifluoromethoxy | Y¹ is N, Y² is CH, |
| | chloro | | | Y³ is CH |
| P.1260 | X¹ is chloro, X² is N, X³ is | trifluoromethyl | trifluoromethyl | Y¹ is N, Y² is CH, |
| | chloro | | | Y³ is CH |
| P.1261 | X¹ is trifluoromethyl, X² is | chlorodifluoromethyl | bromo | Y¹ is N, Y² is CH, |
| | N, X³ is trifluoromethyl | | | Y³ is CH |
| P.1262 | X¹ is trifluoromethyl, X² is | chlorodifluoromethyl | chloro | Y¹ is N, Y² is CH, |
| | N, X³ is trifluoromethyl | | | Y³ is CH |
| P.1263 | X¹ is trifluoromethyl, X² is | chlorodifluoromethyl | cyano | Y¹ is N, Y² is CH, |
| | N, X³ is trifluoromethyl | | | Y³ is CH |
| P.1264 | X¹ is trifluoromethyl, X² is | chlorodifluoromethyl | cyclopropyl | Y¹ is N, Y² is CH, |
| | N, X³ is trifluoromethyl | | | Y³ is CH |
| P.1265 | X¹ is trifluoromethyl, X² is | chlorodifluoromethyl | ethyl | Y¹ is N, Y² is CH, |
| | N, X³ is trifluoromethyl | | | Y³ is CH |
| P.1266 | X¹ is trifluoromethyl, X² is | chlorodifluoromethyl | fluoro | Y¹ is N, Y² is CH, |
| | N, X³ is trifluoromethyl | | | Y³ is CH |
| P.1267 | X¹ is trifluoromethyl, X² is | chlorodifluoromethyl | hydrogen | Y¹ is N, Y² is CH, |
| | N, X³ is trifluoromethyl | | | Y³ is CH |
| P.1268 | X¹ is trifluoromethyl, X² is | chlorodifluoromethyl | methoxy | Y¹ is N, Y² is CH, |
| | N, X³ is trifluoromethyl | | | Y³ is CH |
| P.1269 | X¹ is trifluoromethyl, X² is | chlorodifluoromethyl | methyl | Y¹ is N, Y² is CH, |
| | N, X³ is trifluoromethyl | | | Y³ is CH |
| P.1270 | X¹ is trifluoromethyl, X² is | chlorodifluoromethyl | nitro | Y¹ is N, Y² is CH, |
| | N, X³ is trifluoromethyl | | | Y³ is CH |
| P.1271 | X¹ is trifluoromethyl, X² is | chlorodifluoromethyl | trifluoromethoxy | Y¹ is N, Y² is CH, |
| | N, X³ is trifluoromethyl | | | Y³ is CH |
| P.1272 | X¹ is trifluoromethyl, X² is | chlorodifluoromethyl | trifluoromethyl | Y¹ is N, Y² is CH, |
| | N, X³ is trifluoromethyl | | | Y³ is CH |
| P.1273 | X¹ is trifluoromethyl, X² is | difluoromethyl | bromo | Y¹ is N, Y² is CH, |
| | N, X³ is trifluoromethyl | | | Y³ is CH |
| P.1274 | X¹ is trifluoromethyl, X² is | difluoromethyl | chloro | Y¹ is N, Y² is CH, |
| | N, X³ is trifluoromethyl | | | Y³ is CH |
| P.1275 | X¹ is trifluoromethyl, X² is | difluoromethyl | cyano | Y¹ is N, Y² is CH, |
| | N, X³ is trifluoromethyl | | | Y³ is CH |
| P.1276 | X¹ is trifluoromethyl, X² is | difluoromethyl | cyclopropyl | Y¹ is N, Y² is CH, |
| | N, X³ is trifluoromethyl | | | Y³ is CH |
| P.1277 | X¹ is trifluoromethyl, X² is | difluoromethyl | ethyl | Y¹ is N, Y² is CH, |
| | N, X³ is trifluoromethyl | | | Y³ is CH |
| P.1278 | X¹ is trifluoromethyl, X² is | difluoromethyl | fluoro | Y¹ is N, Y² is CH, |
| | N, X³ is trifluoromethyl | | | Y³ is CH |
| P.1279 | X¹ is trifluoromethyl, X² is | difluoromethyl | hydrogen | Y¹ is N, Y² is CH, |
| | N, X³ is trifluoromethyl | | | Y³ is CH |
| P.1280 | X¹ is trifluoromethyl, X² is | difluoromethyl | methoxy | Y¹ is N, Y² is CH, |
| | N, X³ is trifluoromethyl | | | Y³ is CH |
| P.1281 | X¹ is trifluoromethyl, X² is | difluoromethyl | methyl | Y¹ is N, Y² is CH, |
| | N, X³ is trifluoromethyl | | | Y³ is CH |
| P.1282 | X¹ is trifluoromethyl, X² is | difluoromethyl | nitro | Y¹ is N, Y² is CH, |
| | N, X³ is trifluoromethyl | | | Y³ is CH |
| P.1283 | X¹ is trifluoromethyl, X² is | difluoromethyl | trifluoromethoxy | Y¹ is N, Y² is CH, |
| | N, X³ is trifluoromethyl | | | Y³ is CH |
| P.1284 | X¹ is trifluoromethyl, X² is | difluoromethyl | trifluoromethyl | Y¹ is N, Y² is CH, |
| | N, X³ is trifluoromethyl | | | Y³ is CH |
| P.1285 | X¹ is trifluoromethyl, X² is | trifluoromethyl | bromo | Y¹ is N, Y² is CH, |
| | N, X³ is trifluoromethyl | | | Y³ is CH |
| P.1286 | X¹ is trifluoromethyl, X² is | trifluoromethyl | chloro | Y¹ is N, Y² is CH, |
| | N, X³ is trifluoromethyl | | | Y³ is CH |
| P.1287 | X¹ is trifluoromethyl, X² is | trifluoromethyl | cyano | Y¹ is N, Y² is CH, |
| | N, X³ is trifluoromethyl | | | Y³ is CH |
| P.1288 | X¹ is trifluoromethyl, X² is | trifluoromethyl | cyclopropyl | Y¹ is N, Y² is CH, |
| | N, X³ is trifluoromethyl | | | Y³ is CH |
| P.1289 | X¹ is trifluoromethyl, X² is | trifluoromethyl | ethyl | Y¹ is N, Y² is CH, |
| | N, X³ is trifluoromethyl | | | Y³ is CH |
| P.1290 | X¹ is trifluoromethyl, X² is | trifluoromethyl | fluoro | Y¹ is N, Y² is CH, |
| | N, X³ is trifluoromethyl | | | Y³ is CH |
| P.1291 | X¹ is trifluoromethyl, X² is | trifluoromethyl | hydrogen | Y¹ is N, Y² is CH, |
| | N, X³ is trifluoromethyl | | | Y³ is CH |
| P.1292 | X¹ is trifluoromethyl, X² is | trifluoromethyl | methoxy | Y¹ is N, Y² is CH, |
| | N, X³ is trifluoromethyl | | | Y³ is CH |
| P.1293 | X¹ is trifluoromethyl, X² is | trifluoromethyl | methyl | Y¹ is N, Y² is CH, |
| | N, X³ is trifluoromethyl | | | Y³ is CH |
| P.1294 | X¹ is trifluoromethyl, X² is | trifluoromethyl | nitro | Y¹ is N, Y² is CH, |
| | N, X³ is trifluoromethyl | | | Y³ is CH |
| P.1295 | X¹ is trifluoromethyl, X² is | trifluoromethyl | trifluoromethoxy | Y¹ is N, Y² is CH, |
| | N, X³ is trifluoromethyl | | | Y³ is CH |
| P.1296 | X¹ is trifluoromethyl, X² is | trifluoromethyl | trifluoromethyl | Y¹ is N, Y² is CH, |
| | N, X³ is trifluoromethyl | | | Y³ is CH |
| P.1297 | X¹ is chloro, X² is CH, X³ is | chlorodifluoromethyl | bromo | Y¹ is N, Y² is N, |
| | chloro | | | Y³ is CH |
| P.1298 | X¹ is chloro, X² is CH, X³ is | chlorodifluoromethyl | chloro | Y¹ is N, Y² is N, |
| | chloro | | | Y³ is CH |
| P.1299 | X¹ is chloro, X² is CH, X³ is | chlorodifluoromethyl | cyano | Y¹ is N, Y² is N, |
| | chloro | | | Y³ is CH |
| P.1300 | X¹ is chloro, X² is CH, X³ is | chlorodifluoromethyl | cyclopropyl | Y¹ is N, Y² is N, |
| | chloro | | | Y³ is CH |
| P.1301 | X¹ is chloro, X² is CH, X³ is | chlorodifluoromethyl | ethyl | Y¹ is N, Y² is N, |
| | chloro | | | Y³ is CH |
| P.1302 | X¹ is chloro, X² is CH, X³ is | chlorodifluoromethyl | fluoro | Y¹ is N, Y² is N, |
| | chloro | | | Y³ is CH |
| P.1303 | X¹ is chloro, X² is CH, X³ is | chlorodifluoromethyl | hydrogen | Y¹ is N, Y² is N, |
| | chloro | | | Y³ is CH |
| P.1304 | X¹ is chloro, X² is CH, X³ is | chlorodifluoromethyl | methoxy | Y¹ is N, Y² is N, |
| | chloro | | | Y³ is CH |
| P.1305 | X¹ is chloro, X² is CH, X³ is | chlorodifluoromethyl | methyl | Y¹ is N, Y² is N, |
| | chloro | | | Y³ is CH |
| P.1306 | X¹ is chloro, X² is CH, X³ is | chlorodifluoromethyl | nitro | Y¹ is N, Y² is N, |
| | chloro | | | Y³ is CH |
| P.1307 | X¹ is chloro, X² is CH, X³ is | chlorodifluoromethyl | trifluoromethoxy | Y¹ is N, Y² is N, |
| | chloro | | | Y³ is CH |
| P.1308 | X¹ is chloro, X² is CH, X³ is | chlorodifluoromethyl | trifluoromethyl | Y¹ is N, Y² is N, |
| | chloro | | | Y³ is CH |
| P.1309 | X¹ is chloro, X² is CH, X³ is | difluoromethyl | bromo | Y¹ is N, Y² is N, |
| | chloro | | | Y³ is CH |
| P.1310 | X¹ is chloro, X² is CH, X³ is | difluoromethyl | chloro | Y¹ is N, Y² is N, |
| | chloro | | | Y³ is CH |
| P.1311 | X¹ is chloro, X² is CH, X³ is | difluoromethyl | cyano | Y¹ is N, Y² is N, |
| | chloro | | | Y³ is CH |
| P.1312 | X¹ is chloro, X² is CH, X³ is | difluoromethyl | cyclopropyl | Y¹ is N, Y² is N, |
| | chloro | | | Y³ is CH |
| P.1313 | X¹ is chloro, X² is CH, X³ is | difluoromethyl | ethyl | Y¹ is N, Y² is N, |
| | chloro | | | Y³ is CH |
| P.1314 | X¹ is chloro, X² is CH, X³ is | difluoromethyl | fluoro | Y¹ is N, Y² is N, |
| | chloro | | | Y³ is CH |
| P.1315 | X¹ is chloro, X² is CH, X³ is | difluoromethyl | hydrogen | Y¹ is N, Y² is N, |
| | chloro | | | Y³ is CH |
| P.1316 | X¹ is chloro, X² is CH, X³ is | difluoromethyl | methoxy | Y¹ is N, Y² is N, |
| | chloro | | | Y³ is CH |
| P.1317 | X¹ is chloro, X² is CH, X³ is | difluoromethyl | methyl | Y¹ is N, Y² is N, |
| | chloro | | | Y³ is CH |
| P.1318 | X¹ is chloro, X² is CH, X³ is | difluoromethyl | nitro | Y¹ is N, Y² is N, |
| | chloro | | | Y³ is CH |
| P.1319 | X¹ is chloro, X² is CH, X³ is | difluoromethyl | trifluoromethoxy | Y¹ is N, Y² is N, |
| | chloro | | | Y³ is CH |
| P.1320 | X¹ is chloro, X² is CH, X³ is | difluoromethyl | trifluoromethyl | Y¹ is N, Y² is N, |
| | chloro | | | Y³ is CH |
| P.1321 | X¹ is chloro, X² is CH, X³ is | trifluoromethyl | bromo | Y¹ is N, Y² is N, |
| | chloro | | | Y³ is CH |
| P.1322 | X¹ is chloro, X² is CH, X³ is | trifluoromethyl | chloro | Y¹ is N, Y² is N, |
| | chloro | | | Y³ is CH |
| P.1323 | X¹ is chloro, X² is CH, X³ is | trifluoromethyl | cyano | Y¹ is N, Y² is N, |
| | chloro | | | Y³ is CH |
| P.1324 | X¹ is chloro, X² is CH, X³ is | trifluoromethyl | cyclopropyl | Y¹ is N, Y² is N, |
| | chloro | | | Y³ is CH |
| P.1325 | X¹ is chloro, X² is CH, X³ is | trifluoromethyl | ethyl | Y¹ is N, Y² is N, |
| | chloro | | | Y³ is CH |
| P.1326 | X¹ is chloro, X² is CH, X³ is | trifluoromethyl | fluoro | Y¹ is N, Y² is N, |
| | chloro | | | Y³ is CH |
| P.1327 | X¹ is chloro, X² is CH, X³ is | trifluoromethyl | hydrogen | Y¹ is N, Y² is N, |
| | chloro | | | Y³ is CH |
| P.1328 | X¹ is chloro, X² is CH, X³ is | trifluoromethyl | methoxy | Y¹ is N, Y² is N, |
| | chloro | | | Y³ is CH |
| P.1329 | X¹ is chloro, X² is CH, X³ is | trifluoromethyl | methyl | Y¹ is N, Y² is N, |
| | chloro | | | Y³ is CH |
| P.1330 | X¹ is chloro, X² is CH, X³ is | trifluoromethyl | nitro | Y¹ is N, Y² is N, |
| | chloro | | | Y³ is CH |
| P.1331 | X¹ is chloro, X² is CH, X³ is | trifluoromethyl | trifluoromethoxy | Y¹ is N, Y² is N, |
| | chloro | | | Y³ is CH |
| P.1332 | X¹ is chloro, X² is CH, X³ is | trifluoromethyl | trifluoromethyl | Y¹ is N, Y² is N, |
| | chloro | | | Y³ is CH |
| P.1333 | X¹ is chloro, X² is C—F, X³ is | chlorodifluoromethyl | bromo | Y¹ is N, Y² is N, |
| | hydrogen | | | Y³ is CH |
| P.1334 | X¹ is chloro, X² is C—F, X³ is | chlorodifluoromethyl | chloro | Y¹ is N, Y² is N, |
| | hydrogen | | | Y³ is CH |
| P.1335 | X¹ is chloro, X² is C—F, X³ is | chlorodifluoromethyl | cyano | Y¹ is N, Y² is N, |
| | hydrogen | | | Y³ is CH |
| P.1336 | X¹ is chloro, X² is C—F, X³ is | chlorodifluoromethyl | cyclopropyl | Y¹ is N, Y² is N, |
| | hydrogen | | | Y³ is CH |
| P.1337 | X¹ is chloro, X² is C—F, X³ is | chlorodifluoromethyl | ethyl | Y¹ is N, Y² is N, |
| | hydrogen | | | Y³ is CH |
| P.1338 | X¹ is chloro, X² is C—F, X³ is | chlorodifluoromethyl | fluoro | Y¹ is N, Y² is N, |
| | hydrogen | | | Y³ is CH |
| P.1339 | X¹ is chloro, X² is C—F, X³ is | chlorodifluoromethyl | hydrogen | Y¹ is N, Y² is N, |
| | hydrogen | | | Y³ is CH |
| P.1340 | X¹ is chloro, X² is C—F, X³ is | chlorodifluoromethyl | methoxy | Y¹ is N, Y² is N, |
| | hydrogen | | | Y³ is CH |
| P.1341 | X¹ is chloro, X² is C—F, X³ is | chlorodifluoromethyl | methyl | Y¹ is N, Y² is N, |
| | hydrogen | | | Y³ is CH |
| P.1342 | X¹ is chloro, X² is C—F, X³ is | chlorodifluoromethyl | nitro | Y¹ is N, Y² is N, |
| | hydrogen | | | Y³ is CH |
| P.1343 | X¹ is chloro, X² is C—F, X³ is | chlorodifluoromethyl | trifluoromethoxy | Y¹ is N, Y² is N, |
| | hydrogen | | | Y³ is CH |
| P.1344 | X¹ is chloro, X² is C—F, X³ is | chlorodifluoromethyl | trifluoromethyl | Y¹ is N, Y² is N, |
| | hydrogen | | | Y³ is CH |
| P.1345 | X¹ is chloro, X² is C—F, X³ is | difluoromethyl | bromo | Y¹ is N, Y² is N, |
| | hydrogen | | | Y³ is CH |
| P.1346 | X¹ is chloro, X² is C—F, X³ is | difluoromethyl | chloro | Y¹ is N, Y² is N, |
| | hydrogen | | | Y³ is CH |
| P.1347 | X¹ is chloro, X² is C—F, X³ is | difluoromethyl | cyano | Y¹ is N, Y² is N, |
| | hydrogen | | | Y³ is CH |
| P.1348 | X¹ is chloro, X² is C—F, X³ is | difluoromethyl | cyclopropyl | Y¹ is N, Y² is N, |
| | hydrogen | | | Y³ is CH |
| P.1349 | X¹ is chloro, X² is C—F, X³ is | difluoromethyl | ethyl | Y¹ is N, Y² is N, |
| | hydrogen | | | Y³ is CH |
| P.1350 | X¹ is chloro, X² is C—F, X³ is | difluoromethyl | fluoro | Y¹ is N, Y² is N, |
| | hydrogen | | | Y³ is CH |
| P.1351 | X¹ is chloro, X² is C—F, X³ is | difluoromethyl | hydrogen | Y¹ is N, Y² is N, |
| | hydrogen | | | Y³ is CH |
| P.1352 | X¹ is chloro, X² is C—F, X³ is | difluoromethyl | methoxy | Y¹ is N, Y² is N, |
| | hydrogen | | | Y³ is CH |
| P.1353 | X¹ is chloro, X² is C—F, X³ is | difluoromethyl | methyl | Y¹ is N, Y² is N, |
| | hydrogen | | | Y³ is CH |
| P.1354 | X¹ is chloro, X² is C—F, X³ is | difluoromethyl | nitro | Y¹ is N, Y² is N, |
| | hydrogen | | | Y³ is CH |
| P.1355 | X¹ is chloro, X² is C—F, X³ is | difluoromethyl | trifluoromethoxy | Y¹ is N, Y² is N, |
| | hydrogen | | | Y³ is CH |
| P.1356 | X¹ is chloro, X² is C—F, X³ is | difluoromethyl | trifluoromethyl | Y¹ is N, Y² is N, |
| | hydrogen | | | Y³ is CH |
| P.1357 | X¹ is chloro, X² is C—F, X³ is | trifluoromethyl | bromo | Y¹ is N, Y² is N, |
| | hydrogen | | | Y³ is CH |
| P.1358 | X¹ is chloro, X² is C—F, X³ is | trifluoromethyl | chloro | Y¹ is N, Y² is N, |
| | hydrogen | | | Y³ is CH |
| P.1359 | X¹ is chloro, X² is C—F, X³ is | trifluoromethyl | cyano | Y¹ is N, Y² is N, |
| | hydrogen | | | Y³ is CH |
| P.1360 | X¹ is chloro, X² is C—F, X³ is | trifluoromethyl | cyclopropyl | Y¹ is N, Y² is N, |
| | hydrogen | | | Y³ is CH |
| P.1361 | X¹ is chloro, X² is C—F, X³ is | trifluoromethyl | ethyl | Y¹ is N, Y² is N, |
| | hydrogen | | | Y³ is CH |
| P.1362 | X¹ is chloro, X² is C—F, X³ is | trifluoromethyl | fluoro | Y¹ is N, Y² is N, |
| | hydrogen | | | Y³ is CH |
| P.1363 | X¹ is chloro, X² is C—F, X³ is | trifluoromethyl | hydrogen | Y¹ is N, Y² is N, |
| | hydrogen | | | Y³ is CH |
| P.1364 | X¹ is chloro, X² is C—F, X³ is | trifluoromethyl | methoxy | Y¹ is N, Y² is N, |
| | hydrogen | | | Y³ is CH |
| P.1365 | X¹ is chloro, X² is C—F, X³ is | trifluoromethyl | methyl | Y¹ is N, Y² is N, |
| | hydrogen | | | Y³ is CH |
| P.1366 | X¹ is chloro, X² is C—F, X³ is | trifluoromethyl | nitro | Y¹ is N, Y² is N, |
| | hydrogen | | | Y³ is CH |
| P.1367 | X¹ is chloro, X² is C—F, X³ is | trifluoromethyl | trifluoromethoxy | Y¹ is N, Y² is N, |
| | hydrogen | | | Y³ is CH |
| P.1368 | X¹ is chloro, X² is C—F, X³ is | trifluoromethyl | trifluoromethyl | Y¹ is N, Y² is N, |
| | hydrogen | | | Y³ is CH |
| P.1369 | X¹ is fluoro, X² is C—Cl, X³ | chlorodifluoromethyl | bromo | Y¹ is N, Y² is N, |
| | is hydrogen | | | Y³ is CH |
| P.1370 | X¹ is fluoro, X² is C—Cl, X³ | chlorodifluoromethyl | chloro | Y¹ is N, Y² is N, |
| | is hydrogen | | | Y³ is CH |
| P.1371 | X¹ is fluoro, X² is C—Cl, X³ | chlorodifluoromethyl | cyano | Y¹ is N, Y² is N, |
| | is hydrogen | | | Y³ is CH |
| P.1372 | X¹ is fluoro, X² is C—Cl, X³ | chlorodifluoromethyl | cyclopropyl | Y¹ is N, Y² is N, |
| | is hydrogen | | | Y³ is CH |
| P.1373 | X¹ is fluoro, X² is C—Cl, X³ | chlorodifluoromethyl | ethyl | Y¹ is N, Y² is N, |
| | is hydrogen | | | Y³ is CH |
| P.1374 | X¹ is fluoro, X² is C—Cl, X³ | chlorodifluoromethyl | fluoro | Y¹ is N, Y² is N, |
| | is hydrogen | | | Y³ is CH |
| P.1375 | X¹ is fluoro, X² is C—Cl, X³ | chlorodifluoromethyl | hydrogen | Y¹ is N, Y² is N, |
| | is hydrogen | | | Y³ is CH |
| P.1376 | X¹ is fluoro, X² is C—Cl, X³ | chlorodifluoromethyl | methoxy | Y¹ is N, Y² is N, |
| | is hydrogen | | | Y³ is CH |
| P.1377 | X¹ is fluoro, X² is C—Cl, X³ | chlorodifluoromethyl | methyl | Y¹ is N, Y² is N, |
| | is hydrogen | | | Y³ is CH |
| P.1378 | X¹ is fluoro, X² is C—Cl, X³ | chlorodifluoromethyl | nitro | Y¹ is N, Y² is N, |
| | is hydrogen | | | Y³ is CH |
| P.1379 | X¹ is fluoro, X² is C—Cl, X³ | chlorodifluoromethyl | trifluoromethoxy | Y¹ is N, Y² is N, |
| | is hydrogen | | | Y³ is CH |
| P.1380 | X¹ is fluoro, X² is C—Cl, X³ | chlorodifluoromethyl | trifluoromethyl | Y¹ is N, Y² is N, |
| | is hydrogen | | | Y³ is CH |
| P.1381 | X¹ is fluoro, X² is C—Cl, X³ | difluoromethyl | bromo | Y¹ is N, Y² is N, |
| | is hydrogen | | | Y³ is CH |
| P.1382 | X¹ is fluoro, X² is C—Cl, X³ | difluoromethyl | chloro | Y¹ is N, Y² is N, |
| | is hydrogen | | | Y³ is CH |
| P.1383 | X¹ is fluoro, X² is C—Cl, X³ | difluoromethyl | cyano | Y¹ is N, Y² is N, |
| | is hydrogen | | | Y³ is CH |
| P.1384 | X¹ is fluoro, X² is C—Cl, X³ | difluoromethyl | cyclopropyl | Y¹ is N, Y² is N, |
| | is hydrogen | | | Y³ is CH |
| P.1385 | X¹ is fluoro, X² is C—Cl, X³ | difluoromethyl | ethyl | Y¹ is N, Y² is N, |
| | is hydrogen | | | Y³ is CH |
| P.1386 | X¹ is fluoro, X² is C—Cl, X³ | difluoromethyl | fluoro | Y¹ is N, Y² is N, |
| | is hydrogen | | | Y³ is CH |
| P.1387 | X¹ is fluoro, X² is C—Cl, X³ | difluoromethyl | hydrogen | Y¹ is N, Y² is N, |
| | is hydrogen | | | Y³ is CH |
| P.1388 | X¹ is fluoro, X² is C—Cl, X³ | difluoromethyl | methoxy | Y¹ is N, Y² is N, |
| | is hydrogen | | | Y³ is CH |
| P.1389 | X¹ is fluoro, X² is C—Cl, X³ | difluoromethyl | methyl | Y¹ is N, Y² is N, |
| | is hydrogen | | | Y³ is CH |
| P.1390 | X¹ is fluoro, X² is C—Cl, X³ | difluoromethyl | nitro | Y¹ is N, Y² is N, |
| | is hydrogen | | | Y³ is CH |
| P.1391 | X¹ is fluoro, X² is C—Cl, X³ | difluoromethyl | trifluoromethoxy | Y¹ is N, Y² is N, |
| | is hydrogen | | | Y³ is CH |
| P.1392 | X¹ is fluoro, X² is C—Cl, X³ | difluoromethyl | trifluoromethyl | Y¹ is N, Y² is N, |
| | is hydrogen | | | Y³ is CH |
| P.1393 | X¹ is fluoro, X² is C—Cl, X³ | trifluoromethyl | bromo | Y¹ is N, Y² is N, |
| | is hydrogen | | | Y³ is CH |
| P.1394 | X¹ is fluoro, X² is C—Cl, X³ | trifluoromethyl | chloro | Y¹ is N, Y² is N, |
| | is hydrogen | | | Y³ is CH |
| P.1395 | X¹ is fluoro, X² is C—Cl, X³ | trifluoromethyl | cyano | Y¹ is N, Y² is N, |
| | is hydrogen | | | Y³ is CH |
| P.1396 | X¹ is fluoro, X² is C—Cl, X³ | trifluoromethyl | cyclopropyl | Y¹ is N, Y² is N, |
| | is hydrogen | | | Y³ is CH |
| P.1397 | X¹ is fluoro, X² is C—Cl, X³ | trifluoromethyl | ethyl | Y¹ is N, Y² is N, |
| | is hydrogen | | | Y³ is CH |
| P.1398 | X¹ is fluoro, X² is C—Cl, X³ | trifluoromethyl | fluoro | Y¹ is N, Y² is N, |
| | is hydrogen | | | Y³ is CH |
| P.1399 | X¹ is fluoro, X² is C—Cl, X³ | trifluoromethyl | hydrogen | Y¹ is N, Y² is N, |
| | is hydrogen | | | Y³ is CH |
| P.1400 | X¹ is fluoro, X² is C—Cl, X³ | trifluoromethyl | methoxy | Y¹ is N, Y² is N, |
| | is hydrogen | | | Y³ is CH |
| P.1401 | X¹ is fluoro, X² is C—Cl, X³ | trifluoromethyl | methyl | Y¹ is N, Y² is N, |
| | is hydrogen | | | Y³ is CH |
| P.1402 | X¹ is fluoro, X² is C—Cl, X³ | trifluoromethyl | nitro | Y¹ is N, Y² is N, |
| | is hydrogen | | | Y³ is CH |
| P.1403 | X¹ is fluoro, X² is C—Cl, X³ | trifluoromethyl | trifluoromethoxy | Y¹ is N, Y² is N, |
| | is hydrogen | | | Y³ is CH |
| P.1404 | X¹ is fluoro, X² is C—Cl, X³ | trifluoromethyl | trifluoromethyl | Y¹ is N, Y² is N, |
| | is hydrogen | | | Y³ is CH |
| P.1405 | X¹ is chloro, X² is C—Cl, X³ | chlorodifluoromethyl | bromo | Y¹ is N, Y² is N, |
| | is hydrogen | | | Y³ is CH |
| P.1406 | X¹ is chloro, X² is C—Cl, X³ | chlorodifluoromethyl | chloro | Y¹ is N, Y² is N, |
| | is hydrogen | | | Y³ is CH |
| P.1407 | X¹ is chloro, X² is C—Cl, X³ | chlorodifluoromethyl | cyano | Y¹ is N, Y² is N, |
| | is hydrogen | | | Y³ is CH |
| P.1408 | X¹ is chloro, X² is C—Cl, X³ | chlorodifluoromethyl | cyclopropyl | Y¹ is N, Y² is N, |
| | is hydrogen | | | Y³ is CH |
| P.1409 | X¹ is chloro, X² is C—Cl, X³ | chlorodifluoromethyl | ethyl | Y¹ is N, Y² is N, |
| | is hydrogen | | | Y³ is CH |
| P.1410 | X¹ is chloro, X² is C—Cl, X³ | chlorodifluoromethyl | fluoro | Y¹ is N, Y² is N, |
| | is hydrogen | | | Y³ is CH |
| P.1411 | X¹ is chloro, X² is C—Cl, X³ | chlorodifluoromethyl | hydrogen | Y¹ is N, Y² is N, |
| | is hydrogen | | | Y³ is CH |
| P.1412 | X¹ is chloro, X² is C—Cl, X³ | chlorodifluoromethyl | methoxy | Y¹ is N, Y² is N, |
| | is hydrogen | | | Y³ is CH |
| P.1413 | X¹ is chloro, X² is C—Cl, X³ | chlorodifluoromethyl | methyl | Y¹ is N, Y² is N, |
| | is hydrogen | | | Y³ is CH |
| P.1414 | X¹ is chloro, X² is C—Cl, X³ | chlorodifluoromethyl | nitro | Y¹ is N, Y² is N, |
| | is hydrogen | | | Y³ is CH |
| P.1415 | X¹ is chloro, X² is C—Cl, X³ | chlorodifluoromethyl | trifluoromethoxy | Y¹ is N, Y² is N, |
| | is hydrogen | | | Y³ is CH |
| P.1416 | X¹ is chloro, X² is C—Cl, X³ | chlorodifluoromethyl | trifluoromethyl | Y¹ is N, Y² is N, |
| | is hydrogen | | | Y³ is CH |
| P.1417 | X¹ is chloro, X² is C—Cl, X³ | difluoromethyl | bromo | Y¹ is N, Y² is N, |
| | is hydrogen | | | Y³ is CH |
| P.1418 | X¹ is chloro, X² is C—Cl, X³ | difluoromethyl | chloro | Y¹ is N, Y² is N, |
| | is hydrogen | | | Y³ is CH |
| P.1419 | X¹ is chloro, X² is C—Cl, X³ | difluoromethyl | cyano | Y¹ is N, Y² is N, |
| | is hydrogen | | | Y³ is CH |
| P.1420 | X¹ is chloro, X² is C—Cl, X³ | difluoromethyl | cyclopropyl | Y¹ is N, Y² is N, |
| | is hydrogen | | | Y³ is CH |
| P.1421 | X¹ is chloro, X² is C—Cl, X³ | difluoromethyl | ethyl | Y¹ is N, Y² is N, |
| | is hydrogen | | | Y³ is CH |
| P.1422 | X¹ is chloro, X² is C—Cl, X³ | difluoromethyl | fluoro | Y¹ is N, Y² is N, |
| | is hydrogen | | | Y³ is CH |
| P.1423 | X¹ is chloro, X² is C—Cl, X³ | difluoromethyl | hydrogen | Y¹ is N, Y² is N, |
| | is hydrogen | | | Y³ is CH |
| P.1424 | X¹ is chloro, X² is C—Cl, X³ | difluoromethyl | methoxy | Y¹ is N, Y² is N, |
| | is hydrogen | | | Y³ is CH |
| P.1425 | X¹ is chloro, X² is C—Cl, X³ | difluoromethyl | methyl | Y¹ is N, Y² is N, |
| | is hydrogen | | | Y³ is CH |
| P.1426 | X¹ is chloro, X² is C—Cl, X³ | difluoromethyl | nitro | Y¹ is N, Y² is N, |
| | is hydrogen | | | Y³ is CH |
| P.1427 | X¹ is chloro, X² is C—Cl, X³ | difluoromethyl | trifluoromethoxy | Y¹ is N, Y² is N, |
| | is hydrogen | | | Y³ is CH |
| P.1428 | X¹ is chloro, X² is C—Cl, X³ | difluoromethyl | trifluoromethyl | Y¹ is N, Y² is N, |
| | is hydrogen | | | Y³ is CH |
| P.1429 | X¹ is chloro, X² is C—Cl, X³ | trifluoromethyl | bromo | Y¹ is N, Y² is N, |
| | is hydrogen | | | Y³ is CH |
| P.1430 | X¹ is chloro, X² is C—Cl, X³ | trifluoromethyl | chloro | Y¹ is N, Y² is N, |
| | is hydrogen | | | Y³ is CH |
| P.1431 | X¹ is chloro, X² is C—Cl, X³ | trifluoromethyl | cyano | Y¹ is N, Y² is N, |
| | is hydrogen | | | Y³ is CH |
| P.1432 | X¹ is chloro, X² is C—Cl, X³ | trifluoromethyl | cyclopropyl | Y¹ is N, Y² is N, |
| | is hydrogen | | | Y³ is CH |
| P.1433 | X¹ is chloro, X² is C—Cl, X³ | trifluoromethyl | ethyl | Y¹ is N, Y² is N, |
| | is hydrogen | | | Y³ is CH |
| P.1434 | X¹ is chloro, X² is C—Cl, X³ | trifluoromethyl | fluoro | Y¹ is N, Y² is N, |
| | is hydrogen | | | Y³ is CH |
| P.1435 | X¹ is chloro, X² is C—Cl, X³ | trifluoromethyl | hydrogen | Y¹ is N, Y² is N, |
| | is hydrogen | | | Y³ is CH |
| P.1436 | X¹ is chloro, X² is C—Cl, X³ | trifluoromethyl | methoxy | Y¹ is N, Y² is N, |
| | is hydrogen | | | Y³ is CH |
| P.1437 | X¹ is chloro, X² is C—Cl, X³ | trifluoromethyl | methyl | Y¹ is N, Y² is N, |
| | is hydrogen | | | Y³ is CH |
| P.1438 | X¹ is chloro, X² is C—Cl, X³ | trifluoromethyl | nitro | Y¹ is N, Y² is N, |
| | is hydrogen | | | Y³ is CH |
| P.1439 | X¹ is chloro, X² is C—Cl, X³ | trifluoromethyl | trifluoromethoxy | Y¹ is N, Y² is N, |
| | is hydrogen | | | Y³ is CH |
| P.1440 | X¹ is chloro, X² is C—Cl, X³ | trifluoromethyl | trifluoromethyl | Y¹ is N, Y² is N, |
| | is hydrogen | | | Y³ is CH |
| P.1441 | X¹ is chloro, X² is C—Br, X³ | chlorodifluoromethyl | bromo | Y¹ is N, Y² is N, |
| | is chloro | | | Y³ is CH |
| P.1442 | X¹ is chloro, X² is C—Br, X³ | chlorodifluoromethyl | chloro | Y¹ is N, Y² is N, |
| | is chloro | | | Y³ is CH |
| P.1443 | X¹ is chloro, X² is C—Br, X³ | chlorodifluoromethyl | cyano | Y¹ is N, Y² is N, |
| | is chloro | | | Y³ is CH |
| P.1444 | X¹ is chloro, X² is C—Br, X³ | chlorodifluoromethyl | cyclopropyl | Y¹ is N, Y² is N, |
| | is chloro | | | Y³ is CH |
| P.1445 | X¹ is chloro, X² is C—Br, X³ | chlorodifluoromethyl | ethyl | Y¹ is N, Y² is N, |
| | is chloro | | | Y³ is CH |
| P.1446 | X¹ is chloro, X² is C—Br, X³ | chlorodifluoromethyl | fluoro | Y¹ is N, Y² is N, |
| | is chloro | | | Y³ is CH |
| P.1447 | X¹ is chloro, X² is C—Br, X³ | chlorodifluoromethyl | hydrogen | Y¹ is N, Y² is N, |
| | is chloro | | | Y³ is CH |
| P.1448 | X¹ is chloro, X² is C—Br, X³ | chlorodifluoromethyl | methoxy | Y¹ is N, Y² is N, |
| | is chloro | | | Y³ is CH |
| P.1449 | X¹ is chloro, X² is C—Br, X³ | chlorodifluoromethyl | methyl | Y¹ is N, Y² is N, |
| | is chloro | | | Y³ is CH |
| P.1450 | X¹ is chloro, X² is C—Br, X³ | chlorodifluoromethyl | nitro | Y¹ is N, Y² is N, |
| | is chloro | | | Y³ is CH |
| P.1451 | X¹ is chloro, X² is C—Br, X³ | chlorodifluoromethyl | trifluoromethoxy | Y¹ is N, Y² is N, |
| | is chloro | | | Y³ is CH |
| P.1452 | X¹ is chloro, X² is C—Br, X³ | chlorodifluoromethyl | trifluoromethyl | Y¹ is N, Y² is N, |
| | is chloro | | | Y³ is CH |
| P.1453 | X¹ is chloro, X² is C—Br, X³ | difluoromethyl | bromo | Y¹ is N, Y² is N, |
| | is chloro | | | Y³ is CH |
| P.1454 | X¹ is chloro, X² is C—Br, X³ | difluoromethyl | chloro | Y¹ is N, Y² is N, |
| | is chloro | | | Y³ is CH |
| P.1455 | X¹ is chloro, X² is C—Br, X³ | difluoromethyl | cyano | Y¹ is N, Y² is N, |
| | is chloro | | | Y³ is CH |
| P.1456 | X¹ is chloro, X² is C—Br, X³ | difluoromethyl | cyclopropyl | Y¹ is N, Y² is N, |
| | is chloro | | | Y³ is CH |
| P.1457 | X¹ is chloro, X² is C—Br, X³ | difluoromethyl | ethyl | Y¹ is N, Y² is N, |
| | is chloro | | | Y³ is CH |
| P.1458 | X¹ is chloro, X² is C—Br, X³ | difluoromethyl | fluoro | Y¹ is N, Y² is N, |
| | is chloro | | | Y³ is CH |
| P.1459 | X¹ is chloro, X² is C—Br, X³ | difluoromethyl | hydrogen | Y¹ is N, Y² is N, |
| | is chloro | | | Y³ is CH |
| P.1460 | X¹ is chloro, X² is C—Br, X³ | difluoromethyl | methoxy | Y¹ is N, Y² is N, |
| | is chloro | | | Y³ is CH |
| P.1461 | X¹ is chloro, X² is C—Br, X³ | difluoromethyl | methyl | Y¹ is N, Y² is N, |
| | is chloro | | | Y³ is CH |
| P.1462 | X¹ is chloro, X² is C—Br, X³ | difluoromethyl | nitro | Y¹ is N, Y² is N, |
| | is chloro | | | Y³ is CH |
| P.1463 | X¹ is chloro, X² is C—Br, X³ | difluoromethyl | trifluoromethoxy | Y¹ is N, Y² is N, |
| | is chloro | | | Y³ is CH |
| P.1464 | X¹ is chloro, X² is C—Br, X³ | difluoromethyl | trifluoromethyl | Y¹ is N, Y² is N, |
| | is chloro | | | Y³ is CH |
| P.1465 | X¹ is chloro, X² is C—Br, X³ | trifluoromethyl | bromo | Y¹ is N, Y² is N, |
| | is chloro | | | Y³ is CH |
| P.1466 | X¹ is chloro, X² is C—Br, X³ | trifluoromethyl | chloro | Y¹ is N, Y² is N, |
| | is chloro | | | Y³ is CH |
| P.1467 | X¹ is chloro, X² is C—Br, X³ | trifluoromethyl | cyano | Y¹ is N, Y² is N, |
| | is chloro | | | Y³ is CH |
| P.1468 | X¹ is chloro, X² is C—Br, X³ | trifluoromethyl | cyclopropyl | Y¹ is N, Y² is N, |
| | is chloro | | | Y³ is CH |
| P.1469 | X¹ is chloro, X² is C—Br, X³ | trifluoromethyl | ethyl | Y¹ is N, Y² is N, |
| | is chloro | | | Y³ is CH |
| P.1470 | X¹ is chloro, X² is C—Br, X³ | trifluoromethyl | fluoro | Y¹ is N, Y² is N, |
| | is chloro | | | Y³ is CH |
| P.1471 | X¹ is chloro, X² is C—Br, X³ | trifluoromethyl | hydrogen | Y¹ is N, Y² is N, |
| | is chloro | | | Y³ is CH |
| P.1472 | X¹ is chloro, X² is C—Br, X³ | trifluoromethyl | methoxy | Y¹ is N, Y² is N, |
| | is chloro | | | Y³ is CH |
| P.1473 | X¹ is chloro, X² is C—Br, X³ | trifluoromethyl | methyl | Y¹ is N, Y² is N, |
| | is chloro | | | Y³ is CH |
| P.1474 | X¹ is chloro, X² is C—Br, X³ | trifluoromethyl | nitro | Y¹ is N, Y² is N, |
| | is chloro | | | Y³ is CH |
| P.1475 | X¹ is chloro, X² is C—Br, X³ | trifluoromethyl | trifluoromethoxy | Y¹ is N, Y² is N, |
| | is chloro | | | Y³ is CH |
| P.1476 | X¹ is chloro, X² is C—Br, X³ | trifluoromethyl | trifluoromethyl | Y¹ is N, Y² is N, |
| | is chloro | | | Y³ is CH |
| P.1477 | X¹ is chloro, X² is C—F, X³ is | chlorodifluoromethyl | bromo | Y¹ is N, Y² is N, |
| | chloro | | | Y³ is CH |
| P.1478 | X¹ is chloro, X² is C—F, X³ is | chlorodifluoromethyl | chloro | Y¹ is N, Y² is N, |
| | chloro | | | Y³ is CH |
| P.1479 | X¹ is chloro, X² is C—F, X³ is | chlorodifluoromethyl | cyano | Y¹ is N, Y² is N, |
| | chloro | | | Y³ is CH |
| P.1480 | X¹ is chloro, X² is C—F, X³ is | chlorodifluoromethyl | cyclopropyl | Y¹ is N, Y² is N, |
| | chloro | | | Y³ is CH |
| P.1481 | X¹ is chloro, X² is C—F, X³ is | chlorodifluoromethyl | ethyl | Y¹ is N, Y² is N, |
| | chloro | | | Y³ is CH |
| P.1482 | X¹ is chloro, X² is C—F, X³ is | chlorodifluoromethyl | fluoro | Y¹ is N, Y² is N, |
| | chloro | | | Y³ is CH |
| P.1483 | X¹ is chloro, X² is C—F, X³ is | chlorodifluoromethyl | hydrogen | Y¹ is N, Y² is N, |
| | chloro | | | Y³ is CH |
| P.1484 | X¹ is chloro, X² is C—F, X³ is | chlorodifluoromethyl | methoxy | Y¹ is N, Y² is N, |
| | chloro | | | Y³ is CH |
| P.1485 | X¹ is chloro, X² is C—F, X³ is | chlorodifluoromethyl | methyl | Y¹ is N, Y² is N, |
| | chloro | | | Y³ is CH |
| P.1486 | X¹ is chloro, X² is C—F, X³ is | chlorodifluoromethyl | nitro | Y¹ is N, Y² is N, |
| | chloro | | | Y³ is CH |
| P.1487 | X¹ is chloro, X² is C—F, X³ is | chlorodifluoromethyl | trifluoromethoxy | Y¹ is N, Y² is N, |
| | chloro | | | Y³ is CH |
| P.1488 | X¹ is chloro, X² is C—F, X³ is | chlorodifluoromethyl | trifluoromethyl | Y¹ is N, Y² is N, |
| | chloro | | | Y³ is CH |
| P.1489 | X¹ is chloro, X² is C—F, X³ is | difluoromethyl | bromo | Y¹ is N, Y² is N, |
| | chloro | | | Y³ is CH |
| P.1490 | X¹ is chloro, X² is C—F, X³ is | difluoromethyl | chloro | Y¹ is N, Y² is N, |
| | chloro | | | Y³ is CH |
| P.1491 | X¹ is chloro, X² is C—F, X³ is | difluoromethyl | cyano | Y¹ is N, Y² is N, |
| | chloro | | | Y³ is CH |
| P.1492 | X¹ is chloro, X² is C—F, X³ is | difluoromethyl | cyclopropyl | Y¹ is N, Y² is N, |
| | chloro | | | Y³ is CH |
| P.1493 | X¹ is chloro, X² is C—F, X³ is | difluoromethyl | ethyl | Y¹ is N, Y² is N, |
| | chloro | | | Y³ is CH |
| P.1494 | X¹ is chloro, X² is C—F, X³ is | difluoromethyl | fluoro | Y¹ is N, Y² is N, |
| | chloro | | | Y³ is CH |
| P.1495 | X¹ is chloro, X² is C—F, X³ is | difluoromethyl | hydrogen | Y¹ is N, Y² is N, |
| | chloro | | | Y³ is CH |
| P.1496 | X¹ is chloro, X² is C—F, X³ is | difluoromethyl | methoxy | Y¹ is N, Y² is N, |
| | chloro | | | Y³ is CH |
| P.1497 | X¹ is chloro, X² is C—F, X³ is | difluoromethyl | methyl | Y¹ is N, Y² is N, |
| | chloro | | | Y³ is CH |
| P.1498 | X¹ is chloro, X² is C—F, X³ is | difluoromethyl | nitro | Y¹ is N, Y² is N, |
| | chloro | | | Y³ is CH |
| P.1499 | X¹ is chloro, X² is C—F, X³ is | difluoromethyl | trifluoromethoxy | Y¹ is N, Y² is N, |
| | chloro | | | Y³ is CH |
| P.1500 | X¹ is chloro, X² is C—F, X³ is | difluoromethyl | trifluoromethyl | Y¹ is N, Y² is N, |
| | chloro | | | Y³ is CH |
| P.1501 | X¹ is chloro, X² is C—F, X³ is | trifluoromethyl | bromo | Y¹ is N, Y² is N, |
| | chloro | | | Y³ is CH |
| P.1502 | X¹ is chloro, X² is C—F, X³ is | trifluoromethyl | chloro | Y¹ is N, Y² is N, |
| | chloro | | | Y³ is CH |
| P.1503 | X¹ is chloro, X² is C—F, X³ is | trifluoromethyl | cyano | Y¹ is N, Y² is N, |
| | chloro | | | Y³ is CH |
| P.1504 | X¹ is chloro, X² is C—F, X³ is | trifluoromethyl | cyclopropyl | Y¹ is N, Y² is N, |
| | chloro | | | Y³ is CH |
| P.1505 | X¹ is chloro, X² is C—F, X³ is | trifluoromethyl | ethyl | Y¹ is N, Y² is N, |
| | chloro | | | Y³ is CH |
| P.1506 | X¹ is chloro, X² is C—F, X³ is | trifluoromethyl | fluoro | Y¹ is N, Y² is N, |
| | chloro | | | Y³ is CH |
| P.1507 | X¹ is chloro, X² is C—F, X³ is | trifluoromethyl | hydrogen | Y¹ is N, Y² is N, |
| | chloro | | | Y³ is CH |
| P.1508 | X¹ is chloro, X² is C—F, X³ is | trifluoromethyl | methoxy | Y¹ is N, Y² is N, |
| | chloro | | | Y³ is CH |
| P.1509 | X¹ is chloro, X² is C—F, X³ is | trifluoromethyl | methyl | Y¹ is N, Y² is N, |
| | chloro | | | Y³ is CH |
| P.1510 | X¹ is chloro, X² is C—F, X³ is | trifluoromethyl | nitro | Y¹ is N, Y² is N, |
| | chloro | | | Y³ is CH |
| P.1511 | X¹ is chloro, X² is C—F, X³ is | trifluoromethyl | trifluoromethoxy | Y¹ is N, Y² is N, |
| | chloro | | | Y³ is CH |
| P.1512 | X¹ is chloro, X² is C—F, X³ is | trifluoromethyl | trifluoromethyl | Y¹ is N, Y² is N, |
| | chloro | | | Y³ is CH |
| P.1513 | X¹ is chloro, X² is C—Cl, X³ | chlorodifluoromethyl | bromo | Y¹ is N, Y² is N, |
| | is chloro | | | Y³ is CH |
| P.1514 | X¹ is chloro, X² is C—Cl, X³ | chlorodifluoromethyl | chloro | Y¹ is N, Y² is N, |
| | is chloro | | | Y³ is CH |
| P.1515 | X¹ is chloro, X² is C—Cl, X³ | chlorodifluoromethyl | cyano | Y¹ is N, Y² is N, |
| | is chloro | | | Y³ is CH |
| P.1516 | X¹ is chloro, X² is C—Cl, X³ | chlorodifluoromethyl | cyclopropyl | Y¹ is N, Y² is N, |
| | is chloro | | | Y³ is CH |
| P.1517 | X¹ is chloro, X² is C—Cl, X³ | chlorodifluoromethyl | ethyl | Y¹ is N, Y² is N, |
| | is chloro | | | Y³ is CH |
| P.1518 | X¹ is chloro, X² is C—Cl, X³ | chlorodifluoromethyl | fluoro | Y¹ is N, Y² is N, |
| | is chloro | | | Y³ is CH |
| P.1519 | X¹ is chloro, X² is C—Cl, X³ | chlorodifluoromethyl | hydrogen | Y¹ is N, Y² is N, |
| | is chloro | | | Y³ is CH |
| P.1520 | X¹ is chloro, X² is C—Cl, X³ | chlorodifluoromethyl | methoxy | Y¹ is N, Y² is N, |
| | is chloro | | | Y³ is CH |
| P.1521 | X¹ is chloro, X² is C—Cl, X³ | chlorodifluoromethyl | methyl | Y¹ is N, Y² is N, |
| | is chloro | | | Y³ is CH |
| P.1522 | X¹ is chloro, X² is C—Cl, X³ | chlorodifluoromethyl | nitro | Y¹ is N, Y² is N, |
| | is chloro | | | Y³ is CH |
| P.1523 | X¹ is chloro, X² is C—Cl, X³ | chlorodifluoromethyl | trifluoromethoxy | Y¹ is N, Y² is N, |
| | is chloro | | | Y³ is CH |
| P.1524 | X¹ is chloro, X² is C—Cl, X³ | chlorodifluoromethyl | trifluoromethyl | Y¹ is N, Y² is N, |
| | is chloro | | | Y³ is CH |
| P.1525 | X¹ is chloro, X² is C—Cl, X³ | difluoromethyl | bromo | Y¹ is N, Y² is N, |
| | is chloro | | | Y³ is CH |
| P.1526 | X¹ is chloro, X² is C—Cl, X³ | difluoromethyl | chloro | Y¹ is N, Y² is N, |
| | is chloro | | | Y³ is CH |
| P.1527 | X¹ is chloro, X² is C—Cl, X³ | difluoromethyl | cyano | Y¹ is N, Y² is N, |
| | is chloro | | | Y³ is CH |
| P.1528 | X¹ is chloro, X² is C—Cl, X³ | difluoromethyl | cyclopropyl | Y¹ is N, Y² is N, |
| | is chloro | | | Y³ is CH |
| P.1529 | X¹ is chloro, X² is C—Cl, X³ | difluoromethyl | ethyl | Y¹ is N, Y² is N, |
| | is chloro | | | Y³ is CH |
| P.1530 | X¹ is chloro, X² is C—Cl, X³ | difluoromethyl | fluoro | Y¹ is N, Y² is N, |
| | is chloro | | | Y³ is CH |
| P.1531 | X¹ is chloro, X² is C—Cl, X³ | difluoromethyl | hydrogen | Y¹ is N, Y² is N, |
| | is chloro | | | Y³ is CH |
| P.1532 | X¹ is chloro, X² is C—Cl, X³ | difluoromethyl | methoxy | Y¹ is N, Y² is N, |
| | is chloro | | | Y³ is CH |
| P.1533 | X¹ is chloro, X² is C—Cl, X³ | difluoromethyl | methyl | Y¹ is N, Y² is N, |
| | is chloro | | | Y³ is CH |
| P.1534 | X¹ is chloro, X² is C—Cl, X³ | difluoromethyl | nitro | Y¹ is N, Y² is N, |
| | is chloro | | | Y³ is CH |
| P.1535 | X¹ is chloro, X² is C—Cl, X³ | difluoromethyl | trifluoromethoxy | Y¹ is N, Y² is N, |
| | is chloro | | | Y³ is CH |
| P.1536 | X¹ is chloro, X² is C—Cl, X³ | difluoromethyl | trifluoromethyl | Y¹ is N, Y² is N, |
| | is chloro | | | Y³ is CH |
| P.1537 | X¹ is chloro, X² is C—Cl, X³ | trifluoromethyl | bromo | Y¹ is N, Y² is N, |
| | is chloro | | | Y³ is CH |
| P.1538 | X¹ is chloro, X² is C—Cl, X³ | trifluoromethyl | chloro | Y¹ is N, Y² is N, |
| | is chloro | | | Y³ is CH |
| P.1539 | X¹ is chloro, X² is C—Cl, X³ | trifluoromethyl | cyano | Y¹ is N, Y² is N, |
| | is chloro | | | Y³ is CH |
| P.1540 | X¹ is chloro, X² is C—Cl, X³ | trifluoromethyl | cyclopropyl | Y¹ is N, Y² is N, |
| | is chloro | | | Y³ is CH |
| P.1541 | X¹ is chloro, X² is C—Cl, X³ | trifluoromethyl | ethyl | Y¹ is N, Y² is N, |
| | is chloro | | | Y³ is CH |
| P.1542 | X¹ is chloro, X² is C—Cl, X³ | trifluoromethyl | fluoro | Y¹ is N, Y² is N, |
| | is chloro | | | Y³ is CH |
| P.1543 | X¹ is chloro, X² is C—Cl, X³ | trifluoromethyl | hydrogen | Y¹ is N, Y² is N, |
| | is chloro | | | Y³ is CH |
| P.1544 | X¹ is chloro, X² is C—Cl, X³ | trifluoromethyl | methoxy | Y¹ is N, Y² is N, |
| | is chloro | | | Y³ is CH |
| P.1545 | X¹ is chloro, X² is C—Cl, X³ | trifluoromethyl | methyl | Y¹ is N, Y² is N, |
| | is chloro | | | Y³ is CH |
| P.1546 | X¹ is chloro, X² is C—Cl, X³ | trifluoromethyl | nitro | Y¹ is N, Y² is N, |
| | is chloro | | | Y³ is CH |
| P.1547 | X¹ is chloro, X² is C—Cl, X³ | trifluoromethyl | trifluoromethoxy | Y¹ is N, Y² is N, |
| | is chloro | | | Y³ is CH |
| P.1548 | X¹ is chloro, X² is C—Cl, X³ | trifluoromethyl | trifluoromethyl | Y¹ is N, Y² is N, |
| | is chloro | | | Y³ is CH |
| P.1549 | X¹ is chloro, X² is C—I, X³ is | chlorodifluoromethyl | bromo | Y¹ is N, Y² is N, |
| | chloro | | | Y³ is CH |
| P.1550 | X¹ is chloro, X² is C—I, X³ is | chlorodifluoromethyl | chloro | Y¹ is N, Y² is N, |
| | chloro | | | Y³ is CH |
| P.1551 | X¹ is chloro, X² is C—I, X³ is | chlorodifluoromethyl | cyano | Y¹ is N, Y² is N, |
| | chloro | | | Y³ is CH |
| P.1552 | X¹ is chloro, X² is C—I, X³ is | chlorodifluoromethyl | cyclopropyl | Y¹ is N, Y² is N, |
| | chloro | | | Y³ is CH |
| P.1553 | X¹ is chloro, X² is C—I, X³ is | chlorodifluoromethyl | ethyl | Y¹ is N, Y² is N, |
| | chloro | | | Y³ is CH |
| P.1554 | X¹ is chloro, X² is C—I, X³ is | chlorodifluoromethyl | fluoro | Y¹ is N, Y² is N, |
| | chloro | | | Y³ is CH |
| P.1555 | X¹ is chloro, X² is C—I, X³ is | chlorodifluoromethyl | hydrogen | Y¹ is N, Y² is N, |
| | chloro | | | Y³ is CH |
| P.1556 | X¹ is chloro, X² is C—I, X³ is | chlorodifluoromethyl | methoxy | Y¹ is N, Y² is N, |
| | chloro | | | Y³ is CH |
| P.1557 | X¹ is chloro, X² is C—I, X³ is | chlorodifluoromethyl | methyl | Y¹ is N, Y² is N, |
| | chloro | | | Y³ is CH |
| P.1558 | X¹ is chloro, X² is C—I, X³ is | chlorodifluoromethyl | nitro | Y¹ is N, Y² is N, |
| | chloro | | | Y³ is CH |
| P.1559 | X¹ is chloro, X² is C—I, X³ is | chlorodifluoromethyl | trifluoromethoxy | Y¹ is N, Y² is N, |
| | chloro | | | Y³ is CH |
| P.1560 | X¹ is chloro, X² is C—I, X³ is | chlorodifluoromethyl | trifluoromethyl | Y¹ is N, Y² is N, |
| | chloro | | | Y³ is CH |
| P.1561 | X¹ is chloro, X² is C—I, X³ is | difluoromethyl | bromo | Y¹ is N, Y² is N, |
| | chloro | | | Y³ is CH |
| P.1562 | X¹ is chloro, X² is C—I, X³ is | difluoromethyl | chloro | Y¹ is N, Y² is N, |
| | chloro | | | Y³ is CH |
| P.1563 | X¹ is chloro, X² is C—I, X³ is | difluoromethyl | cyano | Y¹ is N, Y² is N, |
| | chloro | | | Y³ is CH |
| P.1564 | X¹ is chloro, X² is C—I, X³ is | difluoromethyl | cyclopropyl | Y¹ is N, Y² is N, |
| | chloro | | | Y³ is CH |
| P.1565 | X¹ is chloro, X² is C—I, X³ is | difluoromethyl | ethyl | Y¹ is N, Y² is N, |
| | chloro | | | Y³ is CH |
| P.1566 | X¹ is chloro, X² is C—I, X³ is | difluoromethyl | fluoro | Y¹ is N, Y² is N, |
| | chloro | | | Y³ is CH |
| P.1567 | X¹ is chloro, X² is C—I, X³ is | difluoromethyl | hydrogen | Y¹ is N, Y² is N, |
| | chloro | | | Y³ is CH |
| P.1568 | X¹ is chloro, X² is C—I, X³ is | difluoromethyl | methoxy | Y¹ is N, Y² is N, |
| | chloro | | | Y³ is CH |
| P.1569 | X¹ is chloro, X² is C—I, X³ is | difluoromethyl | methyl | Y¹ is N, Y² is N, |
| | chloro | | | Y³ is CH |
| P.1570 | X¹ is chloro, X² is C—I, X³ is | difluoromethyl | nitro | Y¹ is N, Y² is N, |
| | chloro | | | Y³ is CH |
| P.1571 | X¹ is chloro, X² is C—I, X³ is | difluoromethyl | trifluoromethoxy | Y¹ is N, Y² is N, |
| | chloro | | | Y³ is CH |
| P.1572 | X¹ is chloro, X² is C—I, X³ is | difluoromethyl | trifluoromethyl | Y¹ is N, Y² is N, |
| | chloro | | | Y³ is CH |
| P.1573 | X¹ is chloro, X² is C—I, X³ is | trifluoromethyl | bromo | Y¹ is N, Y² is N, |
| | chloro | | | Y³ is CH |
| P.1574 | X¹ is chloro, X² is C—I, X³ is | trifluoromethyl | chloro | Y¹ is N, Y² is N, |
| | chloro | | | Y³ is CH |
| P.1575 | X¹ is chloro, X² is C—I, X³ is | trifluoromethyl | cyano | Y¹ is N, Y² is N, |
| | chloro | | | Y³ is CH |
| P.1576 | X¹ is chloro, X² is C—I, X³ is | trifluoromethyl | cyclopropyl | Y¹ is N, Y² is N, |
| | chloro | | | Y³ is CH |
| P.1577 | X¹ is chloro, X² is C—I, X³ is | trifluoromethyl | ethyl | Y¹ is N, Y² is N, |
| | chloro | | | Y³ is CH |
| P.1578 | X¹ is chloro, X² is C—I, X³ is | trifluoromethyl | fluoro | Y¹ is N, Y² is N, |
| | chloro | | | Y³ is CH |
| P.1579 | X¹ is chloro, X² is C—I, X³ is | trifluoromethyl | hydrogen | Y¹ is N, Y² is N, |
| | chloro | | | Y³ is CH |
| P.1580 | X¹ is chloro, X² is C—I, X³ is | trifluoromethyl | methoxy | Y¹ is N, Y² is N, |
| | chloro | | | Y³ is CH |
| P.1581 | X¹ is chloro, X² is C—I, X³ is | trifluoromethyl | methyl | Y¹ is N, Y² is N, |
| | chloro | | | Y³ is CH |
| P.1582 | X¹ is chloro, X² is C—I, X³ is | trifluoromethyl | nitro | Y¹ is N, Y² is N, |
| | chloro | | | Y³ is CH |
| P.1583 | X¹ is chloro, X² is C—I, X³ is | trifluoromethyl | trifluoromethoxy | Y¹ is N, Y² is N, |
| | chloro | | | Y³ is CH |
| P.1584 | X¹ is chloro, X² is C—I, X³ is | trifluoromethyl | trifluoromethyl | Y¹ is N, Y² is N, |
| | chloro | | | Y³ is CH |
| P.1585 | X¹ is fluoro, X² is C—F, X³ is | chlorodifluoromethyl | bromo | Y¹ is N, Y² is N, |
| | fluoro | | | Y³ is CH |
| P.1586 | X¹ is fluoro, X² is C—F, X³ is | chlorodifluoromethyl | chloro | Y¹ is N, Y² is N, |
| | fluoro | | | Y³ is CH |
| P.1587 | X¹ is fluoro, X² is C—F, X³ is | chlorodifluoromethyl | cyano | Y¹ is N, Y² is N, |
| | fluoro | | | Y³ is CH |
| P.1588 | X¹ is fluoro, X² is C—F, X³ is | chlorodifluoromethyl | cyclopropyl | Y¹ is N, Y² is N, |
| | fluoro | | | Y³ is CH |
| P.1589 | X¹ is fluoro, X² is C—F, X³ is | chlorodifluoromethyl | ethyl | Y¹ is N, Y² is N, |
| | fluoro | | | Y³ is CH |
| P.1590 | X¹ is fluoro, X² is C—F, X³ is | chlorodifluoromethyl | fluoro | Y¹ is N, Y² is N, |
| | fluoro | | | Y³ is CH |
| P.1591 | X¹ is fluoro, X² is C—F, X³ is | chlorodifluoromethyl | hydrogen | Y¹ is N, Y² is N, |
| | fluoro | | | Y³ is CH |
| P.1592 | X¹ is fluoro, X² is C—F, X³ is | chlorodifluoromethyl | methoxy | Y¹ is N, Y² is N, |
| | fluoro | | | Y³ is CH |
| P.1593 | X¹ is fluoro, X² is C—F, X³ is | chlorodifluoromethyl | methyl | Y¹ is N, Y² is N, |
| | fluoro | | | Y³ is CH |
| P.1594 | X¹ is fluoro, X² is C—F, X³ is | chlorodifluoromethyl | nitro | Y¹ is N, Y² is N, |
| | fluoro | | | Y³ is CH |
| P.1595 | X¹ is fluoro, X² is C—F, X³ is | chlorodifluoromethyl | trifluoromethoxy | Y¹ is N, Y² is N, |
| | fluoro | | | Y³ is CH |
| P.1596 | X¹ is fluoro, X² is C—F, X³ is | chlorodifluoromethyl | trifluoromethyl | Y¹ is N, Y² is N, |
| | fluoro | | | Y³ is CH |
| P.1597 | X¹ is fluoro, X² is C—F, X³ is | difluoromethyl | bromo | Y¹ is N, Y² is N, |
| | fluoro | | | Y³ is CH |
| P.1598 | X¹ is fluoro, X² is C—F, X³ is | difluoromethyl | chloro | Y¹ is N, Y² is N, |
| | fluoro | | | Y³ is CH |
| P.1599 | X¹ is fluoro, X² is C—F, X³ is | difluoromethyl | cyano | Y¹ is N, Y² is N, |
| | fluoro | | | Y³ is CH |
| P.1600 | X¹ is fluoro, X² is C—F, X³ is | difluoromethyl | cyclopropyl | Y¹ is N, Y² is N, |
| | fluoro | | | Y³ is CH |
| P.1601 | X¹ is fluoro, X² is C—F, X³ is | difluoromethyl | ethyl | Y¹ is N, Y² is N, |
| | fluoro | | | Y³ is CH |
| P.1602 | X¹ is fluoro, X² is C—F, X³ is | difluoromethyl | fluoro | Y¹ is N, Y² is N, |
| | fluoro | | | Y³ is CH |
| P.1603 | X¹ is fluoro, X² is C—F, X³ is | difluoromethyl | hydrogen | Y¹ is N, Y² is N, |
| | fluoro | | | Y³ is CH |
| P.1604 | X¹ is fluoro, X² is C—F, X³ is | difluoromethyl | methoxy | Y¹ is N, Y² is N, |
| | fluoro | | | Y³ is CH |
| P.1605 | X¹ is fluoro, X² is C—F, X³ is | difluoromethyl | methyl | Y¹ is N, Y² is N, |
| | fluoro | | | Y³ is CH |
| P.1606 | X¹ is fluoro, X² is C—F, X³ is | difluoromethyl | nitro | Y¹ is N, Y² is N, |
| | fluoro | | | Y³ is CH |
| P.1607 | X¹ is fluoro, X² is C—F, X³ is | difluoromethyl | trifluoromethoxy | Y¹ is N, Y² is N, |
| | fluoro | | | Y³ is CH |
| P.1608 | X¹ is fluoro, X² is C—F, X³ is | difluoromethyl | trifluoromethyl | Y¹ is N, Y² is N, |
| | fluoro | | | Y³ is CH |
| P.1609 | X¹ is fluoro, X² is C—F, X³ is | trifluoromethyl | bromo | Y¹ is N, Y² is N, |
| | fluoro | | | Y³ is CH |
| P.1610 | X¹ is fluoro, X² is C—F, X³ is | trifluoromethyl | chloro | Y¹ is N, Y² is N, |
| | fluoro | | | Y³ is CH |
| P.1611 | X¹ is fluoro, X² is C—F, X³ is | trifluoromethyl | cyano | Y¹ is N, Y² is N, |
| | fluoro | | | Y³ is CH |
| P.1612 | X¹ is fluoro, X² is C—F, X³ is | trifluoromethyl | cyclopropyl | Y¹ is N, Y² is N, |
| | fluoro | | | Y³ is CH |
| P.1613 | X¹ is fluoro, X² is C—F, X³ is | trifluoromethyl | ethyl | Y¹ is N, Y² is N, |
| | fluoro | | | Y³ is CH |
| P.1614 | X¹ is fluoro, X² is C—F, X³ is | trifluoromethyl | fluoro | Y¹ is N, Y² is N, |
| | fluoro | | | Y³ is CH |
| P.1615 | X¹ is fluoro, X² is C—F, X³ is | trifluoromethyl | hydrogen | Y¹ is N, Y² is N, |
| | fluoro | | | Y³ is CH |
| P.1616 | X¹ is fluoro, X² is C—F, X³ is | trifluoromethyl | methoxy | Y¹ is N, Y² is N, |
| | fluoro | | | Y³ is CH |
| P.1617 | X¹ is fluoro, X² is C—F, X³ is | trifluoromethyl | methyl | Y¹ is N, Y² is N, |
| | fluoro | | | Y³ is CH |
| P.1618 | X¹ is fluoro, X² is C—F, X³ is | trifluoromethyl | nitro | Y¹ is N, Y² is N, |
| | fluoro | | | Y³ is CH |
| P.1619 | X¹ is fluoro, X² is C—F, X³ is | trifluoromethyl | trifluoromethoxy | Y¹ is N, Y² is N, |
| | fluoro | | | Y³ is CH |
| P.1620 | X¹ is fluoro, X² is C—F, X³ is | trifluoromethyl | trifluoromethyl | Y¹ is N, Y² is N, |
| | fluoro | | | Y³ is CH |
| P.1621 | X¹ is chloro, X² is CH, X³ is | chlorodifluoromethyl | bromo | Y¹ is N, Y² is N, |
| | bromo | | | Y³ is CH |
| P.1622 | X¹ is chloro, X² is CH, X³ is | chlorodifluoromethyl | chloro | Y¹ is N, Y² is N, |
| | bromo | | | Y³ is CH |
| P.1623 | X¹ is chloro, X² is CH, X³ is | chlorodifluoromethyl | cyano | Y¹ is N, Y² is N, |
| | bromo | | | Y³ is CH |
| P.1624 | X¹ is chloro, X² is CH, X³ is | chlorodifluoromethyl | cyclopropyl | Y¹ is N, Y² is N, |
| | bromo | | | Y³ is CH |
| P.1625 | X¹ is chloro, X² is CH, X³ is | chlorodifluoromethyl | ethyl | Y¹ is N, Y² is N, |
| | bromo | | | Y³ is CH |
| P.1626 | X¹ is chloro, X² is CH, X³ is | chlorodifluoromethyl | fluoro | Y¹ is N, Y² is N, |
| | bromo | | | Y³ is CH |
| P.1627 | X¹ is chloro, X² is CH, X³ is | chlorodifluoromethyl | hydrogen | Y¹ is N, Y² is N, |
| | bromo | | | Y³ is CH |
| P.1628 | X¹ is chloro, X² is CH, X³ is | chlorodifluoromethyl | methoxy | Y¹ is N, Y² is N, |
| | bromo | | | Y³ is CH |
| P.1629 | X¹ is chloro, X² is CH, X³ is | chlorodifluoromethyl | methyl | Y¹ is N, Y² is N, |
| | bromo | | | Y³ is CH |
| P.1630 | X¹ is chloro, X² is CH, X³ is | chlorodifluoromethyl | nitro | Y¹ is N, Y² is N, |
| | bromo | | | Y³ is CH |
| P.1631 | X¹ is chloro, X² is CH, X³ is | chlorodifluoromethyl | trifluoromethoxy | Y¹ is N, Y² is N, |
| | bromo | | | Y³ is CH |
| P.1632 | X¹ is chloro, X² is CH, X³ is | chlorodifluoromethyl | trifluoromethyl | Y¹ is N, Y² is N, |
| | bromo | | | Y³ is CH |
| P.1633 | X¹ is chloro, X² is CH, X³ is | difluoromethyl | bromo | Y¹ is N, Y² is N, |
| | bromo | | | Y³ is CH |
| P.1634 | X¹ is chloro, X² is CH, X³ is | difluoromethyl | chloro | Y¹ is N, Y² is N, |
| | bromo | | | Y³ is CH |
| P.1635 | X¹ is chloro, X² is CH, X³ is | difluoromethyl | cyano | Y¹ is N, Y² is N, |
| | bromo | | | Y³ is CH |
| P.1636 | X¹ is chloro, X² is CH, X³ is | difluoromethyl | cyclopropyl | Y¹ is N, Y² is N, |
| | bromo | | | Y³ is CH |
| P.1637 | X¹ is chloro, X² is CH, X³ is | difluoromethyl | ethyl | Y¹ is N, Y² is N, |
| | bromo | | | Y³ is CH |
| P.1638 | X¹ is chloro, X² is CH, X³ is | difluoromethyl | fluoro | Y¹ is N, Y² is N, |
| | bromo | | | Y³ is CH |
| P.1639 | X¹ is chloro, X² is CH, X³ is | difluoromethyl | hydrogen | Y¹ is N, Y² is N, |
| | bromo | | | Y³ is CH |
| P.1640 | X¹ is chloro, X² is CH, X³ is | difluoromethyl | methoxy | Y¹ is N, Y² is N, |
| | bromo | | | Y³ is CH |
| P.1641 | X¹ is chloro, X² is CH, X³ is | difluoromethyl | methyl | Y¹ is N, Y² is N, |
| | bromo | | | Y³ is CH |
| P.1642 | X¹ is chloro, X² is CH, X³ is | difluoromethyl | nitro | Y¹ is N, Y² is N, |
| | bromo | | | Y³ is CH |
| P.1643 | X¹ is chloro, X² is CH, X³ is | difluoromethyl | trifluoromethoxy | Y¹ is N, Y² is N, |
| | bromo | | | Y³ is CH |
| P.1644 | X¹ is chloro, X² is CH, X³ is | difluoromethyl | trifluoromethyl | Y¹ is N, Y² is N, |
| | bromo | | | Y³ is CH |
| P.1645 | X¹ is chloro, X² is CH, X³ is | trifluoromethyl | bromo | Y¹ is N, Y² is N, |
| | bromo | | | Y³ is CH |
| P.1646 | X¹ is chloro, X² is CH, X³ is | trifluoromethyl | chloro | Y¹ is N, Y² is N, |
| | bromo | | | Y³ is CH |
| P.1647 | X¹ is chloro, X² is CH, X³ is | trifluoromethyl | cyano | Y¹ is N, Y² is N, |
| | bromo | | | Y³ is CH |
| P.1648 | X¹ is chloro, X² is CH, X³ is | trifluoromethyl | cyclopropyl | Y¹ is N, Y² is N, |
| | bromo | | | Y³ is CH |
| P.1649 | X¹ is chloro, X² is CH, X³ is | trifluoromethyl | ethyl | Y¹ is N, Y² is N, |
| | bromo | | | Y³ is CH |
| P.1650 | X¹ is chloro, X² is CH, X³ is | trifluoromethyl | fluoro | Y¹ is N, Y² is N, |
| | bromo | | | Y³ is CH |
| P.1651 | X¹ is chloro, X² is CH, X³ is | trifluoromethyl | hydrogen | Y¹ is N, Y² is N, |
| | bromo | | | Y³ is CH |
| P.1652 | X¹ is chloro, X² is CH, X³ is | trifluoromethyl | methoxy | Y¹ is N, Y² is N, |
| | bromo | | | Y³ is CH |
| P.1653 | X¹ is chloro, X² is CH, X³ is | trifluoromethyl | methyl | Y¹ is N, Y² is N, |
| | bromo | | | Y³ is CH |
| P.1654 | X¹ is chloro, X² is CH, X³ is | trifluoromethyl | nitro | Y¹ is N, Y² is N, |
| | bromo | | | Y³ is CH |
| P.1655 | X¹ is chloro, X² is CH, X³ is | trifluoromethyl | trifluoromethoxy | Y¹ is N, Y² is N, |
| | bromo | | | Y³ is CH |
| P.1656 | X¹ is chloro, X² is CH, X³ is | trifluoromethyl | trifluoromethyl | Y¹ is N, Y² is N, |
| | bromo | | | Y³ is CH |
| P.1657 | X¹ is chloro, X² is CH, X³ is | chlorodifluoromethyl | bromo | Y¹ is N, Y² is N, |
| | bromo | | | Y³ is CH |
| P.1658 | X¹ is chloro, X² is CH, X³ is | chlorodifluoromethyl | chloro | Y¹ is N, Y² is N, |
| | bromo | | | Y³ is CH |
| P.1659 | X¹ is chloro, X² is CH, X³ is | chlorodifluoromethyl | cyano | Y¹ is N, Y² is N, |
| | bromo | | | Y³ is CH |
| P.1660 | X¹ is chloro, X² is CH, X³ is | chlorodifluoromethyl | cyclopropyl | Y¹ is N, Y² is N, |
| | bromo | | | Y³ is CH |
| P.1661 | X¹ is chloro, X² is CH, X³ is | chlorodifluoromethyl | ethyl | Y¹ is N, Y² is N, |
| | bromo | | | Y³ is CH |
| P.1662 | X¹ is chloro, X² is CH, X³ is | chlorodifluoromethyl | fluoro | Y¹ is N, Y² is N, |
| | bromo | | | Y³ is CH |
| P.1663 | X¹ is chloro, X² is CH, X³ is | chlorodifluoromethyl | hydrogen | Y¹ is N, Y² is N, |
| | bromo | | | Y³ is CH |
| P.1664 | X¹ is chloro, X² is CH, X³ is | chlorodifluoromethyl | methoxy | Y¹ is N, Y² is N, |
| | bromo | | | Y³ is CH |
| P.1665 | X¹ is chloro, X² is CH, X³ is | chlorodifluoromethyl | methyl | Y¹ is N, Y² is N, |
| | bromo | | | Y³ is CH |
| P.1666 | X¹ is chloro, X² is CH, X³ is | chlorodifluoromethyl | nitro | Y¹ is N, Y² is N, |
| | bromo | | | Y³ is CH |
| P.1667 | X¹ is chloro, X² is CH, X³ is | chlorodifluoromethyl | trifluoromethoxy | Y¹ is N, Y² is N, |
| | bromo | | | Y³ is CH |
| P.1668 | X¹ is chloro, X² is CH, X³ is | chlorodifluoromethyl | trifluoromethyl | Y¹ is N, Y² is N, |
| | bromo | | | Y³ is CH |
| P.1669 | X¹ is chloro, X² is CH, X³ is | difluoromethyl | bromo | Y¹ is N, Y² is N, |
| | bromo | | | Y³ is CH |
| P.1670 | X¹ is chloro, X² is CH, X³ is | difluoromethyl | chloro | Y¹ is N, Y² is N, |
| | bromo | | | Y³ is CH |
| P.1671 | X¹ is chloro, X² is CH, X³ is | difluoromethyl | cyano | Y¹ is N, Y² is N, |
| | bromo | | | Y³ is CH |
| P.1672 | X¹ is chloro, X² is CH, X³ is | difluoromethyl | cyclopropyl | Y¹ is N, Y² is N, |
| | bromo | | | Y³ is CH |
| P.1673 | X¹ is chloro, X² is CH, X³ is | difluoromethyl | ethyl | Y¹ is N, Y² is N, |
| | bromo | | | Y³ is CH |
| P.1674 | X¹ is chloro, X² is CH, X³ is | difluoromethyl | fluoro | Y¹ is N, Y² is N, |
| | bromo | | | Y³ is CH |
| P.1675 | X¹ is chloro, X² is CH, X³ is | difluoromethyl | hydrogen | Y¹ is N, Y² is N, |
| | bromo | | | Y³ is CH |
| P.1676 | X¹ is chloro, X² is CH, X³ is | difluoromethyl | methoxy | Y¹ is N, Y² is N, |
| | bromo | | | Y³ is CH |
| P.1677 | X¹ is chloro, X² is CH, X³ is | difluoromethyl | methyl | Y¹ is N, Y² is N, |
| | bromo | | | Y³ is CH |
| P.1678 | X¹ is chloro, X² is CH, X³ is | difluoromethyl | nitro | Y¹ is N, Y² is N, |
| | bromo | | | Y³ is CH |
| P.1679 | X¹ is chloro, X² is CH, X³ is | difluoromethyl | trifluoromethoxy | Y¹ is N, Y² is N, |
| | bromo | | | Y³ is CH |
| P.1680 | X¹ is chloro, X² is CH, X³ is | difluoromethyl | trifluoromethyl | Y¹ is N, Y² is N, |
| | bromo | | | Y³ is CH |
| P.1681 | X¹ is chloro, X² is CH, X³ is | trifluoromethyl | bromo | Y¹ is N, Y² is N, |
| | bromo | | | Y³ is CH |
| P.1682 | X¹ is chloro, X² is CH, X³ is | trifluoromethyl | chloro | Y¹ is N, Y² is N, |
| | bromo | | | Y³ is CH |
| P.1683 | X¹ is chloro, X² is CH, X³ is | trifluoromethyl | cyano | Y¹ is N, Y² is N, |
| | bromo | | | Y³ is CH |
| P.1684 | X¹ is chloro, X² is CH, X³ is | trifluoromethyl | cyclopropyl | Y¹ is N, Y² is N, |
| | bromo | | | Y³ is CH |
| P.1685 | X¹ is chloro, X² is CH, X³ is | trifluoromethyl | ethyl | Y¹ is N, Y² is N, |
| | bromo | | | Y³ is CH |
| P.1686 | X¹ is chloro, X² is CH, X³ is | trifluoromethyl | fluoro | Y¹ is N, Y² is N, |
| | bromo | | | Y³ is CH |
| P.1687 | X¹ is chloro, X² is CH, X³ is | trifluoromethyl | hydrogen | Y¹ is N, Y² is N, |
| | bromo | | | Y³ is CH |
| P.1688 | X¹ is chloro, X² is CH, X³ is | trifluoromethyl | methoxy | Y¹ is N, Y² is N, |
| | bromo | | | Y³ is CH |
| P.1689 | X¹ is chloro, X² is CH, X³ is | trifluoromethyl | methyl | Y¹ is N, Y² is N, |
| | bromo | | | Y³ is CH |
| P.1690 | X¹ is chloro, X² is CH, X³ is | trifluoromethyl | nitro | Y¹ is N, Y² is N, |
| | bromo | | | Y³ is CH |
| P.1691 | X¹ is chloro, X² is CH, X³ is | trifluoromethyl | trifluoromethoxy | Y¹ is N, Y² is N, |
| | bromo | | | Y³ is CH |
| P.1692 | X¹ is chloro, X² is CH, X³ is | trifluoromethyl | trifluoromethyl | Y¹ is N, Y² is N, |
| | bromo | | | Y³ is CH |
| P.1693 | X¹ is chloro, X² is CH, X³ is | chlorodifluoromethyl | bromo | Y¹ is N, Y² is N, |
| | bromo | | | Y³ is CH |
| P.1694 | X¹ is chloro, X² is CH, X³ is | chlorodifluoromethyl | chloro | Y¹ is N, Y² is N, |
| | bromo | | | Y³ is CH |
| P.1695 | X¹ is chloro, X² is CH, X³ is | chlorodifluoromethyl | cyano | Y¹ is N, Y² is N, |
| | bromo | | | Y³ is CH |
| P.1696 | X¹ is chloro, X² is CH, X³ is | chlorodifluoromethyl | cyclopropyl | Y¹ is N, Y² is N, |
| | bromo | | | Y³ is CH |
| P.1697 | X¹ is chloro, X² is CH, X³ is | chlorodifluoromethyl | ethyl | Y¹ is N, Y² is N, |
| | bromo | | | Y³ is CH |
| P.1698 | X¹ is chloro, X² is CH, X³ is | chlorodifluoromethyl | fluoro | Y¹ is N, Y² is N, |
| | bromo | | | Y³ is CH |
| P.1699 | X¹ is chloro, X² is CH, X³ is | chlorodifluoromethyl | hydrogen | Y¹ is N, Y² is N, |
| | bromo | | | Y³ is CH |
| P.1700 | X¹ is chloro, X² is CH, X³ is | chlorodifluoromethyl | methoxy | Y¹ is N, Y² is N, |
| | bromo | | | Y³ is CH |
| P.1701 | X¹ is chloro, X² is CH, X³ is | chlorodifluoromethyl | methyl | Y¹ is N, Y² is N, |
| | bromo | | | Y³ is CH |
| P.1702 | X¹ is chloro, X² is CH, X³ is | chlorodifluoromethyl | nitro | Y¹ is N, Y² is N, |
| | bromo | | | Y³ is CH |
| P.1703 | X¹ is chloro, X² is CH, X³ is | chlorodifluoromethyl | trifluoromethoxy | Y¹ is N, Y² is N, |
| | bromo | | | Y³ is CH |
| P.1704 | X¹ is chloro, X² is CH, X³ is | chlorodifluoromethyl | trifluoromethyl | Y¹ is N, Y² is N, |
| | bromo | | | Y³ is CH |
| P.1705 | X¹ is chloro, X² is CH, X³ is | difluoromethyl | bromo | Y¹ is N, Y² is N, |
| | bromo | | | Y³ is CH |
| P.1706 | X¹ is chloro, X² is CH, X³ is | difluoromethyl | chloro | Y¹ is N, Y² is N, |
| | bromo | | | Y³ is CH |
| P.1707 | X¹ is chloro, X² is CH, X³ is | difluoromethyl | cyano | Y¹ is N, Y² is N, |
| | bromo | | | Y³ is CH |
| P.1708 | X¹ is chloro, X² is CH, X³ is | difluoromethyl | cyclopropyl | Y¹ is N, Y² is N, |
| | bromo | | | Y³ is CH |
| P.1709 | X¹ is chloro, X² is CH, X³ is | difluoromethyl | ethyl | Y¹ is N, Y² is N, |
| | bromo | | | Y³ is CH |
| P.1710 | X¹ is chloro, X² is CH, X³ is | difluoromethyl | fluoro | Y¹ is N, Y² is N, |
| | bromo | | | Y³ is CH |
| P.1711 | X¹ is chloro, X² is CH, X³ is | difluoromethyl | hydrogen | Y¹ is N, Y² is N, |
| | bromo | | | Y³ is CH |
| P.1712 | X¹ is chloro, X² is CH, X³ is | difluoromethyl | methoxy | Y¹ is N, Y² is N, |
| | bromo | | | Y³ is CH |
| P.1713 | X¹ is chloro, X² is CH, X³ is | difluoromethyl | methyl | Y¹ is N, Y² is N, |
| | bromo | | | Y³ is CH |
| P.1714 | X¹ is chloro, X² is CH, X³ is | difluoromethyl | nitro | Y¹ is N, Y² is N, |
| | bromo | | | Y³ is CH |
| P.1715 | X¹ is chloro, X² is CH, X³ is | difluoromethyl | trifluoromethoxy | Y¹ is N, Y² is N, |
| | bromo | | | Y³ is CH |
| P.1716 | X¹ is chloro, X² is CH, X³ is | difluoromethyl | trifluoromethyl | Y¹ is N, Y² is N, |
| | bromo | | | Y³ is CH |
| P.1717 | X¹ is chloro, X² is CH, X³ is | trifluoromethyl | bromo | Y¹ is N, Y² is N, |
| | bromo | | | Y³ is CH |
| P.1718 | X¹ is chloro, X² is CH, X³ is | trifluoromethyl | chloro | Y¹ is N, Y² is N, |
| | bromo | | | Y³ is CH |
| P.1719 | X¹ is chloro, X² is CH, X³ is | trifluoromethyl | cyano | Y¹ is N, Y² is N, |
| | bromo | | | Y³ is CH |
| P.1720 | X¹ is chloro, X² is CH, X³ is | trifluoromethyl | cyclopropyl | Y¹ is N, Y² is N, |
| | bromo | | | Y³ is CH |
| P.1721 | X¹ is chloro, X² is CH, X³ is | trifluoromethyl | ethyl | Y¹ is N, Y² is N, |
| | bromo | | | Y³ is CH |
| P.1722 | X¹ is chloro, X² is CH, X³ is | trifluoromethyl | fluoro | Y¹ is N, Y² is N, |
| | bromo | | | Y³ is CH |
| P.1723 | X¹ is chloro, X² is CH, X³ is | trifluoromethyl | hydrogen | Y¹ is N, Y² is N, |
| | bromo | | | Y³ is CH |
| P.1724 | X¹ is chloro, X² is CH, X³ is | trifluoromethyl | methoxy | Y¹ is N, Y² is N, |
| | bromo | | | Y³ is CH |
| P.1725 | X¹ is chloro, X² is CH, X³ is | trifluoromethyl | methyl | Y¹ is N, Y² is N, |
| | bromo | | | Y³ is CH |
| P.1726 | X¹ is chloro, X² is CH, X³ is | trifluoromethyl | nitro | Y¹ is N, Y² is N, |
| | bromo | | | Y³ is CH |
| P.1727 | X¹ is chloro, X² is CH, X³ is | trifluoromethyl | trifluoromethoxy | Y¹ is N, Y² is N, |
| | bromo | | | Y³ is CH |
| P.1728 | X¹ is chloro, X² is CH, X³ is | trifluoromethyl | trifluoromethyl | Y¹ is N, Y² is N, |
| | bromo | | | Y³ is CH |
| P.1729 | X¹ is chloro, X² is C—Cl, X³ | chlorodifluoromethyl | bromo | Y¹ is N, Y² is N, |
| | is trifluoromethyl | | | Y³ is CH |
| P.1730 | X¹ is chloro, X² is C—Cl, X³ | chlorodifluoromethyl | chloro | Y¹ is N, Y² is N, |
| | is trifluoromethyl | | | Y³ is CH |
| P.1731 | X¹ is chloro, X² is C—Cl, X³ | chlorodifluoromethyl | cyano | Y¹ is N, Y² is N, |
| | is trifluoromethyl | | | Y³ is CH |
| P.1732 | X¹ is chloro, X² is C—Cl, X³ | chlorodifluoromethyl | cyclopropyl | Y¹ is N, Y² is N, |
| | is trifluoromethyl | | | Y³ is CH |
| P.1733 | X¹ is chloro, X² is C—Cl, X³ | chlorodifluoromethyl | ethyl | Y¹ is N, Y² is N, |
| | is trifluoromethyl | | | Y³ is CH |
| P.1734 | X¹ is chloro, X² is C—Cl, X³ | chlorodifluoromethyl | fluoro | Y¹ is N, Y² is N, |
| | is trifluoromethyl | | | Y³ is CH |
| P.1735 | X¹ is chloro, X² is C—Cl, X³ | chlorodifluoromethyl | hydrogen | Y¹ is N, Y² is N, |
| | is trifluoromethyl | | | Y³ is CH |
| P.1736 | X¹ is chloro, X² is C—Cl, X³ | chlorodifluoromethyl | methoxy | Y¹ is N, Y² is N, |
| | is trifluoromethyl | | | Y³ is CH |
| P.1737 | X¹ is chloro, X² is C—Cl, X³ | chlorodifluoromethyl | methyl | Y¹ is N, Y² is N, |
| | is trifluoromethyl | | | Y³ is CH |
| P.1738 | X¹ is chloro, X² is C—Cl, X³ | chlorodifluoromethyl | nitro | Y¹ is N, Y² is N, |
| | is trifluoromethyl | | | Y³ is CH |
| P.1739 | X¹ is chloro, X² is C—Cl, X³ | chlorodifluoromethyl | trifluoromethoxy | Y¹ is N, Y² is N, |
| | is trifluoromethyl | | | Y³ is CH |
| P.1740 | X¹ is chloro, X² is C—Cl, X³ | chlorodifluoromethyl | trifluoromethyl | Y¹ is N, Y² is N, |
| | is trifluoromethyl | | | Y³ is CH |
| P.1741 | X¹ is chloro, X² is C—Cl, X³ | difluoromethyl | bromo | Y¹ is N, Y² is N, |
| | is trifluoromethyl | | | Y³ is CH |
| P.1742 | X¹ is chloro, X² is C—Cl, X³ | difluoromethyl | chloro | Y¹ is N, Y² is N, |
| | is trifluoromethyl | | | Y³ is CH |
| P.1743 | X¹ is chloro, X² is C—Cl, X³ | difluoromethyl | cyano | Y¹ is N, Y² is N, |
| | is trifluoromethyl | | | Y³ is CH |
| P.1744 | X¹ is chloro, X² is C—Cl, X³ | difluoromethyl | cyclopropyl | Y¹ is N, Y² is N, |
| | is trifluoromethyl | | | Y³ is CH |
| P.1745 | X¹ is chloro, X² is C—Cl, X³ | difluoromethyl | ethyl | Y¹ is N, Y² is N, |
| | is trifluoromethyl | | | Y³ is CH |
| P.1746 | X¹ is chloro, X² is C—Cl, X³ | difluoromethyl | fluoro | Y¹ is N, Y² is N, |
| | is trifluoromethyl | | | Y³ is CH |
| P.1747 | X¹ is chloro, X² is C—Cl, X³ | difluoromethyl | hydrogen | Y¹ is N, Y² is N, |
| | is trifluoromethyl | | | Y³ is CH |
| P.1748 | X¹ is chloro, X² is C—Cl, X³ | difluoromethyl | methoxy | Y¹ is N, Y² is N, |
| | is trifluoromethyl | | | Y³ is CH |
| P.1749 | X¹ is chloro, X² is C—Cl, X³ | difluoromethyl | methyl | Y¹ is N, Y² is N, |
| | is trifluoromethyl | | | Y³ is CH |
| P.1750 | X¹ is chloro, X² is C—Cl, X³ | difluoromethyl | nitro | Y¹ is N, Y² is N, |
| | is trifluoromethyl | | | Y³ is CH |
| P.1751 | X¹ is chloro, X² is C—Cl, X³ | difluoromethyl | trifluoromethoxy | Y¹ is N, Y² is N, |
| | is trifluoromethyl | | | Y³ is CH |
| P.1752 | X¹ is chloro, X² is C—Cl, X³ | difluoromethyl | trifluoromethyl | Y¹ is N, Y² is N, |
| | is trifluoromethyl | | | Y³ is CH |
| P.1753 | X¹ is chloro, X² is C—Cl, X³ | trifluoromethyl | bromo | Y¹ is N, Y² is N, |
| | is trifluoromethyl | | | Y³ is CH |
| P.1754 | X¹ is chloro, X² is C—Cl, X³ | trifluoromethyl | chloro | Y¹ is N, Y² is N, |
| | is trifluoromethyl | | | Y³ is CH |
| P.1755 | X¹ is chloro, X² is C—Cl, X³ | trifluoromethyl | cyano | Y¹ is N, Y² is N, |
| | is trifluoromethyl | | | Y³ is CH |
| P.1756 | X¹ is chloro, X² is C—Cl, X³ | trifluoromethyl | cyclopropyl | Y¹ is N, Y² is N, |
| | is trifluoromethyl | | | Y³ is CH |
| P.1757 | X¹ is chloro, X² is C—Cl, X³ | trifluoromethyl | ethyl | Y¹ is N, Y² is N, |
| | is trifluoromethyl | | | Y³ is CH |
| P.1758 | X¹ is chloro, X² is C—Cl, X³ | trifluoromethyl | fluoro | Y¹ is N, Y² is N, |
| | is trifluoromethyl | | | Y³ is CH |
| P.1759 | X¹ is chloro, X² is C—Cl, X³ | trifluoromethyl | hydrogen | Y¹ is N, Y² is N, |
| | is trifluoromethyl | | | Y³ is CH |
| P.1760 | X¹ is chloro, X² is C—Cl, X³ | trifluoromethyl | methoxy | Y¹ is N, Y² is N, |
| | is trifluoromethyl | | | Y³ is CH |
| P.1761 | X¹ is chloro, X² is C—Cl, X³ | trifluoromethyl | methyl | Y¹ is N, Y² is N, |
| | is trifluoromethyl | | | Y³ is CH |
| P.1762 | X¹ is chloro, X² is C—Cl, X³ | trifluoromethyl | nitro | Y¹ is N, Y² is N, |
| | is trifluoromethyl | | | Y³ is CH |
| P.1763 | X¹ is chloro, X² is C—Cl, X³ | trifluoromethyl | trifluoromethoxy | Y¹ is N, Y² is N, |
| | is trifluoromethyl | | | Y³ is CH |
| P.1764 | X¹ is chloro, X² is C—Cl, X³ | trifluoromethyl | trifluoromethyl | Y¹ is N, Y² is N, |
| | is trifluoromethyl | | | Y³ is CH |
| P.1765 | X¹ is trifluoromethyl, X² is | chlorodifluoromethyl | bromo | Y¹ is N, Y² is N, |
| | CH, X³ is trifluoromethyl | | | Y³ is CH |
| P.1766 | X¹ is trifluoromethyl, X² is | chlorodifluoromethyl | chloro | Y¹ is N, Y² is N, |
| | CH, X³ is trifluoromethyl | | | Y³ is CH |
| P.1767 | X¹ is trifluoromethyl, X² is | chlorodifluoromethyl | cyano | Y¹ is N, Y² is N, |
| | CH, X³ is trifluoromethyl | | | Y³ is CH |
| P.1768 | X¹ is trifluoromethyl, X² is | chlorodifluoromethyl | cyclopropyl | Y¹ is N, Y² is N, |
| | CH, X³ is trifluoromethyl | | | Y³ is CH |
| P.1769 | X¹ is trifluoromethyl, X² is | chlorodifluoromethyl | ethyl | Y¹ is N, Y² is N, |
| | CH, X³ is trifluoromethyl | | | Y³ is CH |
| P.1770 | X¹ is trifluoromethyl, X² is | chlorodifluoromethyl | fluoro | Y¹ is N, Y² is N, |
| | CH, X³ is trifluoromethyl | | | Y³ is CH |
| P.1771 | X¹ is trifluoromethyl, X² is | chlorodifluoromethyl | hydrogen | Y¹ is N, Y² is N, |
| | CH, X³ is trifluoromethyl | | | Y³ is CH |
| P.1772 | X¹ is trifluoromethyl, X² is | chlorodifluoromethyl | methoxy | Y¹ is N, Y² is N, |
| | CH, X³ is trifluoromethyl | | | Y³ is CH |
| P.1773 | X¹ is trifluoromethyl, X² is | chlorodifluoromethyl | methyl | Y¹ is N, Y² is N, |
| | CH, X³ is trifluoromethyl | | | Y³ is CH |
| P.1774 | X¹ is trifluoromethyl, X² is | chlorodifluoromethyl | nitro | Y¹ is N, Y² is N, |
| | CH, X³ is trifluoromethyl | | | Y³ is CH |
| P.1775 | X¹ is trifluoromethyl, X² is | chlorodifluoromethyl | trifluoromethoxy | Y¹ is N, Y² is N, |
| | CH, X³ is trifluoromethyl | | | Y³ is CH |
| P.1776 | X¹ is trifluoromethyl, X² is | chlorodifluoromethyl | trifluoromethyl | Y¹ is N, Y² is N, |
| | CH, X³ is trifluoromethyl | | | Y³ is CH |
| P.1777 | X¹ is trifluoromethyl, X² is | difluoromethyl | bromo | Y¹ is N, Y² is N, |
| | CH, X³ is trifluoromethyl | | | Y³ is CH |
| P.1778 | X¹ is trifluoromethyl, X² is | difluoromethyl | chloro | Y¹ is N, Y² is N, |
| | CH, X³ is trifluoromethyl | | | Y³ is CH |
| P.1779 | X¹ is trifluoromethyl, X² is | difluoromethyl | cyano | Y¹ is N, Y² is N, |
| | CH, X³ is trifluoromethyl | | | Y³ is CH |
| P.1780 | X¹ is trifluoromethyl, X² is | difluoromethyl | cyclopropyl | Y¹ is N, Y² is N, |
| | CH, X³ is trifluoromethyl | | | Y³ is CH |
| P.1781 | X¹ is trifluoromethyl, X² is | difluoromethyl | ethyl | Y¹ is N, Y² is N, |
| | CH, X³ is trifluoromethyl | | | Y³ is CH |
| P.1782 | X¹ is trifluoromethyl, X² is | difluoromethyl | fluoro | Y¹ is N, Y² is N, |
| | CH, X³ is trifluoromethyl | | | Y³ is CH |
| P.1783 | X¹ is trifluoromethyl, X² is | difluoromethyl | hydrogen | Y¹ is N, Y² is N, |
| | CH, X³ is trifluoromethyl | | | Y³ is CH |
| P.1784 | X¹ is trifluoromethyl, X² is | difluoromethyl | methoxy | Y¹ is N, Y² is N, |
| | CH, X³ is trifluoromethyl | | | Y³ is CH |
| P.1785 | X¹ is trifluoromethyl, X² is | difluoromethyl | methyl | Y¹ is N, Y² is N, |
| | CH, X³ is trifluoromethyl | | | Y³ is CH |
| P.1786 | X¹ is trifluoromethyl, X² is | difluoromethyl | nitro | Y¹ is N, Y² is N, |
| | CH, X³ is trifluoromethyl | | | Y³ is CH |
| P.1787 | X¹ is trifluoromethyl, X² is | difluoromethyl | trifluoromethoxy | Y¹ is N, Y² is N, |
| | CH, X³ is trifluoromethyl | | | Y³ is CH |
| P.1788 | X¹ is trifluoromethyl, X² is | difluoromethyl | trifluoromethyl | Y¹ is N, Y² is N, |
| | CH, X³ is trifluoromethyl | | | Y³ is CH |
| P.1789 | X¹ is trifluoromethyl, X² is | trifluoromethyl | bromo | Y¹ is N, Y² is N, |
| | CH, X³ is trifluoromethyl | | | Y³ is CH |
| P.1790 | X¹ is trifluoromethyl, X² is | trifluoromethyl | chloro | Y¹ is N, Y² is N, |
| | CH, X³ is trifluoromethyl | | | Y³ is CH |
| P.1791 | X¹ is trifluoromethyl, X² is | trifluoromethyl | cyano | Y¹ is N, Y² is N, |
| | CH, X³ is trifluoromethyl | | | Y³ is CH |
| P.1792 | X¹ is trifluoromethyl, X² is | trifluoromethyl | cyclopropyl | Y¹ is N, Y² is N, |
| | CH, X³ is trifluoromethyl | | | Y³ is CH |
| P.1793 | X¹ is trifluoromethyl, X² is | trifluoromethyl | ethyl | Y¹ is N, Y² is N, |
| | CH, X³ is trifluoromethyl | | | Y³ is CH |
| P.1794 | X¹ is trifluoromethyl, X² is | trifluoromethyl | fluoro | Y¹ is N, Y² is N, |
| | CH, X³ is trifluoromethyl | | | Y³ is CH |
| P.1795 | X¹ is trifluoromethyl, X² is | trifluoromethyl | hydrogen | Y¹ is N, Y² is N, |
| | CH, X³ is trifluoromethyl | | | Y³ is CH |
| P.1796 | X¹ is trifluoromethyl, X² is | trifluoromethyl | methoxy | Y¹ is N, Y² is N, |
| | CH, X³ is trifluoromethyl | | | Y³ is CH |
| P.1797 | X¹ is trifluoromethyl, X² is | trifluoromethyl | methyl | Y¹ is N, Y² is N, |
| | CH, X³ is trifluoromethyl | | | Y³ is CH |
| P.1798 | X¹ is trifluoromethyl, X² is | trifluoromethyl | nitro | Y¹ is N, Y² is N, |
| | CH, X³ is trifluoromethyl | | | Y³ is CH |
| P.1799 | X¹ is trifluoromethyl, X² is | trifluoromethyl | trifluoromethoxy | Y¹ is N, Y² is N, |
| | CH, X³ is trifluoromethyl | | | Y³ is CH |
| P.1800 | X¹ is trifluoromethyl, X² is | trifluoromethyl | trifluoromethyl | Y¹ is N, Y² is N, |
| | CH, X³ is trifluoromethyl | | | Y³ is CH |
| P.1801 | X¹ is trifluoromethyl, X² is | chlorodifluoromethyl | bromo | Y¹ is N, Y² is N, |
| | CH, X³ is trifluoromethyl | | | Y³ is CH |
| P.1802 | X¹ is trifluoromethyl, X² is | chlorodifluoromethyl | chloro | Y¹ is N, Y² is N, |
| | CH, X³ is trifluoromethyl | | | Y³ is CH |
| P.1803 | X¹ is trifluoromethyl, X² is | chlorodifluoromethyl | cyano | Y¹ is N, Y² is N, |
| | CH, X³ is trifluoromethyl | | | Y³ is CH |
| P.1804 | X¹ is trifluoromethyl, X² is | chlorodifluoromethyl | cyclopropyl | Y¹ is N, Y² is N, |
| | CH, X³ is trifluoromethyl | | | Y³ is CH |
| P.1805 | X¹ is trifluoromethyl, X² is | chlorodifluoromethyl | ethyl | Y¹ is N, Y² is N, |
| | CH, X³ is trifluoromethyl | | | Y³ is CH |
| P.1806 | X¹ is trifluoromethyl, X² is | chlorodifluoromethyl | fluoro | Y¹ is N, Y² is N, |
| | CH, X³ is trifluoromethyl | | | Y³ is CH |
| P.1807 | X¹ is trifluoromethyl, X² is | chlorodifluoromethyl | hydrogen | Y¹ is N, Y² is N, |
| | CH, X³ is trifluoromethyl | | | Y³ is CH |
| P.1808 | X¹ is trifluoromethyl, X² is | chlorodifluoromethyl | methoxy | Y¹ is N, Y² is N, |
| | CH, X³ is trifluoromethyl | | | Y³ is CH |
| P.1809 | X¹ is trifluoromethyl, X² is | chlorodifluoromethyl | methyl | Y¹ is N, Y² is N, |
| | CH, X³ is trifluoromethyl | | | Y³ is CH |
| P.1810 | X¹ is trifluoromethyl, X² is | chlorodifluoromethyl | nitro | Y¹ is N, Y² is N, |
| | CH, X³ is trifluoromethyl | | | Y³ is CH |
| P.1811 | X¹ is trifluoromethyl, X² is | chlorodifluoromethyl | trifluoromethoxy | Y¹ is N, Y² is N, |
| | CH, X³ is trifluoromethyl | | | Y³ is CH |
| P.1812 | X¹ is trifluoromethyl, X² is | chlorodifluoromethyl | trifluoromethyl | Y¹ is N, Y² is N, |
| | CH, X³ is trifluoromethyl | | | Y³ is CH |
| P.1813 | X¹ is trifluoromethyl, X² is | difluoromethyl | bromo | Y¹ is N, Y² is N, |
| | CH, X³ is trifluoromethyl | | | Y³ is CH |
| P.1814 | X¹ is trifluoromethyl, X² is | difluoromethyl | chloro | Y¹ is N, Y² is N, |
| | CH, X³ is trifluoromethyl | | | Y³ is CH |
| P.1815 | X¹ is trifluoromethyl, X² is | difluoromethyl | cyano | Y¹ is N, Y² is N, |
| | CH, X³ is trifluoromethyl | | | Y³ is CH |
| P.1816 | X¹ is trifluoromethyl, X² is | difluoromethyl | cyclopropyl | Y¹ is N, Y² is N, |
| | CH, X³ is trifluoromethyl | | | Y³ is CH |
| P.1817 | X¹ is trifluoromethyl, X² is | difluoromethyl | ethyl | Y¹ is N, Y² is N, |
| | CH, X³ is trifluoromethyl | | | Y³ is CH |
| P.1818 | X¹ is trifluoromethyl, X² is | difluoromethyl | fluoro | Y¹ is N, Y² is N, |
| | CH, X³ is trifluoromethyl | | | Y³ is CH |
| P.1819 | X¹ is trifluoromethyl, X² is | difluoromethyl | hydrogen | Y¹ is N, Y² is N, |
| | CH, X³ is trifluoromethyl | | | Y³ is CH |
| P.1820 | X¹ is trifluoromethyl, X² is | difluoromethyl | methoxy | Y¹ is N, Y² is N, |
| | CH, X³ is trifluoromethyl | | | Y³ is CH |
| P.1821 | X¹ is trifluoromethyl, X² is | difluoromethyl | methyl | Y¹ is N, Y² is N, |
| | CH, X³ is trifluoromethyl | | | Y³ is CH |
| P.1822 | X¹ is trifluoromethyl, X² is | difluoromethyl | nitro | Y¹ is N, Y² is N, |
| | CH, X³ is trifluoromethyl | | | Y³ is CH |
| P.1823 | X¹ is trifluoromethyl, X² is | difluoromethyl | trifluoromethoxy | Y¹ is N, Y² is N, |
| | CH, X³ is trifluoromethyl | | | Y³ is CH |
| P.1824 | X¹ is trifluoromethyl, X² is | difluoromethyl | trifluoromethyl | Y¹ is N, Y² is N, |
| | CH, X³ is trifluoromethyl | | | Y³ is CH |
| P.1825 | X¹ is trifluoromethyl, X² is | trifluoromethyl | bromo | Y¹ is N, Y² is N, |
| | CH, X³ is trifluoromethyl | | | Y³ is CH |
| P.1826 | X¹ is trifluoromethyl, X² is | trifluoromethyl | chloro | Y¹ is N, Y² is N, |
| | CH, X³ is trifluoromethyl | | | Y³ is CH |
| P.1827 | X¹ is trifluoromethyl, X² is | trifluoromethyl | cyano | Y¹ is N, Y² is N, |
| | CH, X³ is trifluoromethyl | | | Y³ is CH |
| P.1828 | X¹ is trifluoromethyl, X² is | trifluoromethyl | cyclopropyl | Y¹ is N, Y² is N, |
| | CH, X³ is trifluoromethyl | | | Y³ is CH |
| P.1829 | X¹ is trifluoromethyl, X² is | trifluoromethyl | ethyl | Y¹ is N, Y² is N, |
| | CH, X³ is trifluoromethyl | | | Y³ is CH |
| P.1830 | X¹ is trifluoromethyl, X² is | trifluoromethyl | fluoro | Y¹ is N, Y² is N, |
| | CH, X³ is trifluoromethyl | | | Y³ is CH |
| P.1831 | X¹ is trifluoromethyl, X² is | trifluoromethyl | hydrogen | Y¹ is N, Y² is N, |
| | CH, X³ is trifluoromethyl | | | Y³ is CH |
| P.1832 | X¹ is trifluoromethyl, X² is | trifluoromethyl | methoxy | Y¹ is N, Y² is N, |
| | CH, X³ is trifluoromethyl | | | Y³ is CH |
| P.1833 | X¹ is trifluoromethyl, X² is | trifluoromethyl | methyl | Y¹ is N, Y² is N, |
| | CH, X³ is trifluoromethyl | | | Y³ is CH |
| P.1834 | X¹ is trifluoromethyl, X² is | trifluoromethyl | nitro | Y¹ is N, Y² is N, |
| | CH, X³ is trifluoromethyl | | | Y³ is CH |
| P.1835 | X¹ is trifluoromethyl, X² is | trifluoromethyl | trifluoromethoxy | Y¹ is N, Y² is N, |
| | CH, X³ is trifluoromethyl | | | Y³ is CH |
| P.1836 | X¹ is trifluoromethyl, X² is | trifluoromethyl | trifluoromethyl | Y¹ is N, Y² is N, |
| | CH, X³ is trifluoromethyl | | | Y³ is CH |
| P.1837 | X¹ is trifluoromethyl, X² is | chlorodifluoromethyl | bromo | Y¹ is N, Y² is N, |
| | CH, X³ is hydrogen | | | Y³ is CH |
| P.1838 | X¹ is trifluoromethyl, X² is | chlorodifluoromethyl | chloro | Y¹ is N, Y² is N, |
| | CH, X³ is hydrogen | | | Y³ is CH |
| P.1839 | X¹ is trifluoromethyl, X² is | chlorodifluoromethyl | cyano | Y¹ is N, Y² is N, |
| | CH, X³ is hydrogen | | | Y³ is CH |
| P.1840 | X¹ is trifluoromethyl, X² is | chlorodifluoromethyl | cyclopropyl | Y¹ is N, Y² is N, |
| | CH, X³ is hydrogen | | | Y³ is CH |
| P.1841 | X¹ is trifluoromethyl, X² is | chlorodifluoromethyl | ethyl | Y¹ is N, Y² is N, |
| | CH, X³ is hydrogen | | | Y³ is CH |
| P.1842 | X¹ is trifluoromethyl, X² is | chlorodifluoromethyl | fluoro | Y¹ is N, Y² is N, |
| | CH, X³ is hydrogen | | | Y³ is CH |
| P.1843 | X¹ is trifluoromethyl, X² is | chlorodifluoromethyl | hydrogen | Y¹ is N, Y² is N, |
| | CH, X³ is hydrogen | | | Y³ is CH |
| P.1844 | X¹ is trifluoromethyl, X² is | chlorodifluoromethyl | methoxy | Y¹ is N, Y² is N, |
| | CH, X³ is hydrogen | | | Y³ is CH |
| P.1845 | X¹ is trifluoromethyl, X² is | chlorodifluoromethyl | methyl | Y¹ is N, Y² is N, |
| | CH, X³ is hydrogen | | | Y³ is CH |
| P.1846 | X¹ is trifluoromethyl, X² is | chlorodifluoromethyl | nitro | Y¹ is N, Y² is N, |
| | CH, X³ is hydrogen | | | Y³ is CH |
| P.1847 | X¹ is trifluoromethyl, X² is | chlorodifluoromethyl | trifluoromethoxy | Y¹ is N, Y² is N, |
| | CH, X³ is hydrogen | | | Y³ is CH |
| P.1848 | X¹ is trifluoromethyl, X² is | chlorodifluoromethyl | trifluoromethyl | Y¹ is N, Y² is N, |
| | CH, X³ is hydrogen | | | Y³ is CH |
| P.1849 | X¹ is trifluoromethyl, X² is | difluoromethyl | bromo | Y¹ is N, Y² is N, |
| | CH, X³ is hydrogen | | | Y³ is CH |
| P.1850 | X¹ is trifluoromethyl, X² is | difluoromethyl | chloro | Y¹ is N, Y² is N, |
| | CH, X³ is hydrogen | | | Y³ is CH |
| P.1851 | X¹ is trifluoromethyl, X² is | difluoromethyl | cyano | Y¹ is N, Y² is N, |
| | CH, X³ is hydrogen | | | Y³ is CH |
| P.1852 | X¹ is trifluoromethyl, X² is | difluoromethyl | cyclopropyl | Y¹ is N, Y² is N, |
| | CH, X³ is hydrogen | | | Y³ is CH |
| P.1853 | X¹ is trifluoromethyl, X² is | difluoromethyl | ethyl | Y¹ is N, Y² is N, |
| | CH, X³ is hydrogen | | | Y³ is CH |
| P.1854 | X¹ is trifluoromethyl, X² is | difluoromethyl | fluoro | Y¹ is N, Y² is N, |
| | CH, X³ is hydrogen | | | Y³ is CH |
| P.1855 | X¹ is trifluoromethyl, X² is | difluoromethyl | hydrogen | Y¹ is N, Y² is N, |
| | CH, X³ is hydrogen | | | Y³ is CH |
| P.1856 | X¹ is trifluoromethyl, X² is | difluoromethyl | methoxy | Y¹ is N, Y² is N, |
| | CH, X³ is hydrogen | | | Y³ is CH |
| P.1857 | X¹ is trifluoromethyl, X² is | difluoromethyl | methyl | Y¹ is N, Y² is N, |
| | CH, X³ is hydrogen | | | Y³ is CH |
| P.1858 | X¹ is trifluoromethyl, X² is | difluoromethyl | nitro | Y¹ is N, Y² is N, |
| | CH, X³ is hydrogen | | | Y³ is CH |
| P.1859 | X¹ is trifluoromethyl, X² is | difluoromethyl | trifluoromethoxy | Y¹ is N, Y² is N, |
| | CH, X³ is hydrogen | | | Y³ is CH |
| P.1860 | X¹ is trifluoromethyl, X² is | difluoromethyl | trifluoromethyl | Y¹ is N, Y² is N, |
| | CH, X³ is hydrogen | | | Y³ is CH |
| P.1861 | X¹ is trifluoromethyl, X² is | trifluoromethyl | bromo | Y¹ is N, Y² is N, |
| | CH, X³ is hydrogen | | | Y³ is CH |
| P.1862 | X¹ is trifluoromethyl, X² is | trifluoromethyl | chloro | Y¹ is N, Y² is N, |
| | CH, X³ is hydrogen | | | Y³ is CH |
| P.1863 | X¹ is trifluoromethyl, X² is | trifluoromethyl | cyano | Y¹ is N, Y² is N, |
| | CH, X³ is hydrogen | | | Y³ is CH |
| P.1864 | X¹ is trifluoromethyl, X² is | trifluoromethyl | cyclopropyl | Y¹ is N, Y² is N, |
| | CH, X³ is hydrogen | | | Y³ is CH |
| P.1865 | X¹ is trifluoromethyl, X² is | trifluoromethyl | ethyl | Y¹ is N, Y² is N, |
| | CH, X³ is hydrogen | | | Y³ is CH |
| P.1866 | X¹ is trifluoromethyl, X² is | trifluoromethyl | fluoro | Y¹ is N, Y² is N, |
| | CH, X³ is hydrogen | | | Y³ is CH |
| P.1867 | X¹ is trifluoromethyl, X² is | trifluoromethyl | hydrogen | Y¹ is N, Y² is N, |
| | CH, X³ is hydrogen | | | Y³ is CH |
| P.1868 | X¹ is trifluoromethyl, X² is | trifluoromethyl | methoxy | Y¹ is N, Y² is N, |
| | CH, X³ is hydrogen | | | Y³ is CH |
| P.1869 | X¹ is trifluoromethyl, X² is | trifluoromethyl | methyl | Y¹ is N, Y² is N, |
| | CH, X³ is hydrogen | | | Y³ is CH |
| P.1870 | X¹ is trifluoromethyl, X² is | trifluoromethyl | nitro | Y¹ is N, Y² is N, |
| | CH, X³ is hydrogen | | | Y³ is CH |
| P.1871 | X¹ is trifluoromethyl, X² is | trifluoromethyl | trifluoromethoxy | Y¹ is N, Y² is N, |
| | CH, X³ is hydrogen | | | Y³ is CH |
| P.1872 | X¹ is trifluoromethyl, X² is | trifluoromethyl | trifluoromethyl | Y¹ is N, Y² is N, |
| | CH, X³ is hydrogen | | | Y³ is CH |
| P.1873 | X¹ is chloro, X² is N, X³ is | chlorodifluoromethyl | bromo | Y¹ is N, Y² is N, |
| | chloro | | | Y³ is CH |
| P.1874 | X¹ is chloro, X² is N, X³ is | chlorodifluoromethyl | chloro | Y¹ is N, Y² is N, |
| | chloro | | | Y³ is CH |
| P.1875 | X¹ is chloro, X² is N, X³ is | chlorodifluoromethyl | cyano | Y¹ is N, Y² is N, |
| | chloro | | | Y³ is CH |
| P.1876 | X¹ is chloro, X² is N, X³ is | chlorodifluoromethyl | cyclopropyl | Y¹ is N, Y² is N, |
| | chloro | | | Y³ is CH |
| P.1877 | X¹ is chloro, X² is N, X³ is | chlorodifluoromethyl | ethyl | Y¹ is N, Y² is N, |
| | chloro | | | Y³ is CH |
| P.1878 | X¹ is chloro, X² is N, X³ is | chlorodifluoromethyl | fluoro | Y¹ is N, Y² is N, |
| | chloro | | | Y³ is CH |
| P.1879 | X¹ is chloro, X² is N, X³ is | chlorodifluoromethyl | hydrogen | Y¹ is N, Y² is N, |
| | chloro | | | Y³ is CH |
| P.1880 | X¹ is chloro, X² is N, X³ is | chlorodifluoromethyl | methoxy | Y¹ is N, Y² is N, |
| | chloro | | | Y³ is CH |
| P.1881 | X¹ is chloro, X² is N, X³ is | chlorodifluoromethyl | methyl | Y¹ is N, Y² is N, |
| | chloro | | | Y³ is CH |
| P.1882 | X¹ is chloro, X² is N, X³ is | chlorodifluoromethyl | nitro | Y¹ is N, Y² is N, |
| | chloro | | | Y³ is CH |
| P.1883 | X¹ is chloro, X² is N, X³ is | chlorodifluoromethyl | trifluoromethoxy | Y¹ is N, Y² is N, |
| | chloro | | | Y³ is CH |
| P.1884 | X¹ is chloro, X² is N, X³ is | chlorodifluoromethyl | trifluoromethyl | Y¹ is N, Y² is N, |
| | chloro | | | Y³ is CH |
| P.1885 | X¹ is chloro, X² is N, X³ is | difluoromethyl | bromo | Y¹ is N, Y² is N, |
| | chloro | | | Y³ is CH |
| P.1886 | X¹ is chloro, X² is N, X³ is | difluoromethyl | chloro | Y¹ is N, Y² is N, |
| | chloro | | | Y³ is CH |
| P.1887 | X¹ is chloro, X² is N, X³ is | difluoromethyl | cyano | Y¹ is N, Y² is N, |
| | chloro | | | Y³ is CH |
| P.1888 | X¹ is chloro, X² is N, X³ is | difluoromethyl | cyclopropyl | Y¹ is N, Y² is N, |
| | chloro | | | Y³ is CH |
| P.1889 | X¹ is chloro, X² is N, X³ is | difluoromethyl | ethyl | Y¹ is N, Y² is N, |
| | chloro | | | Y³ is CH |
| P.1890 | X¹ is chloro, X² is N, X³ is | difluoromethyl | fluoro | Y¹ is N, Y² is N, |
| | chloro | | | Y³ is CH |
| P.1891 | X¹ is chloro, X² is N, X³ is | difluoromethyl | hydrogen | Y¹ is N, Y² is N, |
| | chloro | | | Y³ is CH |
| P.1892 | X¹ is chloro, X² is N, X³ is | difluoromethyl | methoxy | Y¹ is N, Y² is N, |
| | chloro | | | Y³ is CH |
| P.1893 | X¹ is chloro, X² is N, X³ is | difluoromethyl | methyl | Y¹ is N, Y² is N, |
| | chloro | | | Y³ is CH |
| P.1894 | X¹ is chloro, X² is N, X³ is | difluoromethyl | nitro | Y¹ is N, Y² is N, |
| | chloro | | | Y³ is CH |
| P.1895 | X¹ is chloro, X² is N, X³ is | difluoromethyl | trifluoromethoxy | Y¹ is N, Y² is N, |
| | chloro | | | Y³ is CH |
| P.1896 | X¹ is chloro, X² is N, X³ is | difluoromethyl | trifluoromethyl | Y¹ is N, Y² is N, |
| | chloro | | | Y³ is CH |
| P.1897 | X¹ is chloro, X² is N, X³ is | trifluoromethyl | bromo | Y¹ is N, Y² is N, |
| | chloro | | | Y³ is CH |
| P.1898 | X¹ is chloro, X² is N, X³ is | trifluoromethyl | chloro | Y¹ is N, Y² is N, |
| | chloro | | | Y³ is CH |
| P.1899 | X¹ is chloro, X² is N, X³ is | trifluoromethyl | cyano | Y¹ is N, Y² is N, |
| | chloro | | | Y³ is CH |
| P.1900 | X¹ is chloro, X² is N, X³ is | trifluoromethyl | cyclopropyl | Y¹ is N, Y² is N, |
| | chloro | | | Y³ is CH |
| P.1901 | X¹ is chloro, X² is N, X³ is | trifluoromethyl | ethyl | Y¹ is N, Y² is N, |
| | chloro | | | Y³ is CH |
| P.1902 | X¹ is chloro, X² is N, X³ is | trifluoromethyl | fluoro | Y¹ is N, Y² is N, |
| | chloro | | | Y³ is CH |
| P.1903 | X¹ is chloro, X² is N, X³ is | trifluoromethyl | hydrogen | Y¹ is N, Y² is N, |
| | chloro | | | Y³ is CH |
| P.1904 | X¹ is chloro, X² is N, X³ is | trifluoromethyl | methoxy | Y¹ is N, Y² is N, |
| | chloro | | | Y³ is CH |
| P.1905 | X¹ is chloro, X² is N, X³ is | trifluoromethyl | methyl | Y¹ is N, Y² is N, |
| | chloro | | | Y³ is CH |
| P.1906 | X¹ is chloro, X² is N, X³ is | trifluoromethyl | nitro | Y¹ is N, Y² is N, |
| | chloro | | | Y³ is CH |
| P.1907 | X¹ is chloro, X² is N, X³ is | trifluoromethyl | trifluoromethoxy | Y¹ is N, Y² is N, |
| | chloro | | | Y³ is CH |
| P.1908 | X¹ is chloro, X² is N, X³ is | trifluoromethyl | trifluoromethyl | Y¹ is N, Y² is N, |
| | chloro | | | Y³ is CH |
| P.1909 | X¹ is trifluoromethyl, X² is | chlorodifluoromethyl | bromo | Y¹ is N, Y² is N, |
| | N, X³ is trifluoromethyl | | | Y³ is CH |
| P.1910 | X¹ is trifluoromethyl, X² is | chlorodifluoromethyl | chloro | Y¹ is N, Y² is N, |
| | N, X³ is trifluoromethyl | | | Y³ is CH |
| P.1911 | X¹ is trifluoromethyl, X² is | chlorodifluoromethyl | cyano | Y¹ is N, Y² is N, |
| | N, X³ is trifluoromethyl | | | Y³ is CH |
| P.1912 | X¹ is trifluoromethyl, X² is | chlorodifluoromethyl | cyclopropyl | Y¹ is N, Y² is N, |
| | N, X³ is trifluoromethyl | | | Y³ is CH |
| P.1913 | X¹ is trifluoromethyl, X² is | chlorodifluoromethyl | ethyl | Y¹ is N, Y² is N, |
| | N, X³ is trifluoromethyl | | | Y³ is CH |
| P.1914 | X¹ is trifluoromethyl, X² is | chlorodifluoromethyl | fluoro | Y¹ is N, Y² is N, |
| | N, X³ is trifluoromethyl | | | Y³ is CH |
| P.1915 | X¹ is trifluoromethyl, X² is | chlorodifluoromethyl | hydrogen | Y¹ is N, Y² is N, |
| | N, X³ is trifluoromethyl | | | Y³ is CH |
| P.1916 | X¹ is trifluoromethyl, X² is | chlorodifluoromethyl | methoxy | Y¹ is N, Y² is N, |
| | N, X³ is trifluoromethyl | | | Y³ is CH |
| P.1917 | X¹ is trifluoromethyl, X² is | chlorodifluoromethyl | methyl | Y¹ is N, Y² is N, |
| | N, X³ is trifluoromethyl | | | Y³ is CH |
| P.1918 | X¹ is trifluoromethyl, X² is | chlorodifluoromethyl | nitro | Y¹ is N, Y² is N, |
| | N, X³ is trifluoromethyl | | | Y³ is CH |
| P.1919 | X¹ is trifluoromethyl, X² is | chlorodifluoromethyl | trifluoromethoxy | Y¹ is N, Y² is N, |
| | N, X³ is trifluoromethyl | | | Y³ is CH |
| P.1920 | X¹ is trifluoromethyl, X² is | chlorodifluoromethyl | trifluoromethyl | Y¹ is N, Y² is N, |
| | N, X³ is trifluoromethyl | | | Y³ is CH |
| P.1921 | X¹ is trifluoromethyl, X² is | difluoromethyl | bromo | Y¹ is N, Y² is N, |
| | N, X³ is trifluoromethyl | | | Y³ is CH |
| P.1922 | X¹ is trifluoromethyl, X² is | difluoromethyl | chloro | Y¹ is N, Y² is N, |
| | N, X³ is trifluoromethyl | | | Y³ is CH |
| P.1923 | X¹ is trifluoromethyl, X² is | difluoromethyl | cyano | Y¹ is N, Y² is N, |
| | N, X³ is trifluoromethyl | | | Y³ is CH |
| P.1924 | X¹ is trifluoromethyl, X² is | difluoromethyl | cyclopropyl | Y¹ is N, Y² is N, |
| | N, X³ is trifluoromethyl | | | Y³ is CH |
| P.1925 | X¹ is trifluoromethyl, X² is | difluoromethyl | ethyl | Y¹ is N, Y² is N, |
| | N, X³ is trifluoromethyl | | | Y³ is CH |
| P.1926 | X¹ is trifluoromethyl, X² is | difluoromethyl | fluoro | Y¹ is N, Y² is N, |
| | N, X³ is trifluoromethyl | | | Y³ is CH |
| P.1927 | X¹ is trifluoromethyl, X² is | difluoromethyl | hydrogen | Y¹ is N, Y² is N, |
| | N, X³ is trifluoromethyl | | | Y³ is CH |
| P.1928 | X¹ is trifluoromethyl, X² is | difluoromethyl | methoxy | Y¹ is N, Y² is N, |
| | N, X³ is trifluoromethyl | | | Y³ is CH |
| P.1929 | X¹ is trifluoromethyl, X² is | difluoromethyl | methyl | Y¹ is N, Y² is N, |
| | N, X³ is trifluoromethyl | | | Y³ is CH |
| P.1930 | X¹ is trifluoromethyl, X² is | difluoromethyl | nitro | Y¹ is N, Y² is N, |
| | N, X³ is trifluoromethyl | | | Y³ is CH |
| P.1931 | X¹ is trifluoromethyl, X² is | difluoromethyl | trifluoromethoxy | Y¹ is N, Y² is N, |
| | N, X³ is trifluoromethyl | | | Y³ is CH |
| P.1932 | X¹ is trifluoromethyl, X² is | difluoromethyl | trifluoromethyl | Y¹ is N, Y² is N, |
| | N, X³ is trifluoromethyl | | | Y³ is CH |
| P.1933 | X¹ is trifluoromethyl, X² is | trifluoromethyl | bromo | Y¹ is N, Y² is N, |
| | N, X³ is trifluoromethyl | | | Y³ is CH |
| P.1934 | X¹ is trifluoromethyl, X² is | trifluoromethyl | chloro | Y¹ is N, Y² is N, |
| | N, X³ is trifluoromethyl | | | Y³ is CH |
| P.1935 | X¹ is trifluoromethyl, X² is | trifluoromethyl | cyano | Y¹ is N, Y² is N, |
| | N, X³ is trifluoromethyl | | | Y³ is CH |
| P.1936 | X¹ is trifluoromethyl, X² is | trifluoromethyl | cyclopropyl | Y¹ is N, Y² is N, |
| | N, X³ is trifluoromethyl | | | Y³ is CH |
| P.1937 | X¹ is trifluoromethyl, X² is | trifluoromethyl | ethyl | Y¹ is N, Y² is N, |
| | N, X³ is trifluoromethyl | | | Y³ is CH |
| P.1938 | X¹ is trifluoromethyl, X² is | trifluoromethyl | fluoro | Y¹ is N, Y² is N, |
| | N, X³ is trifluoromethyl | | | Y³ is CH |
| P.1939 | X¹ is trifluoromethyl, X² is | trifluoromethyl | hydrogen | Y¹ is N, Y² is N, |
| | N, X³ is trifluoromethyl | | | Y³ is CH |
| P.1940 | X¹ is trifluoromethyl, X² is | trifluoromethyl | methoxy | Y¹ is N, Y² is N, |
| | N, X³ is trifluoromethyl | | | Y³ is CH |
| P.1941 | X¹ is trifluoromethyl, X² is | trifluoromethyl | methyl | Y¹ is N, Y² is N, |
| | N, X³ is trifluoromethyl | | | Y³ is CH |
| P.1942 | X¹ is trifluoromethyl, X² is | trifluoromethyl | nitro | Y¹ is N, Y² is N, |
| | N, X³ is trifluoromethyl | | | Y³ is CH |
| P.1943 | X¹ is trifluoromethyl, X² is | trifluoromethyl | trifluoromethoxy | Y¹ is N, Y² is N, |
| | N, X³ is trifluoromethyl | | | Y³ is CH |
| P.1944 | X¹ is trifluoromethyl, X² is | trifluoromethyl | trifluoromethyl | Y¹ is N, Y² is N, |
| | N, X³ is trifluoromethyl | | | Y³ is CH |
| P.1945 | X¹ is chloro, X² is CH, X³ is | chlorodifluoromethyl | bromo | Y¹ is CH, Y² is N, |
| | chloro | | | Y³ is CH |
| P.1946 | X¹ is chloro, X² is CH, X³ is | chlorodifluoromethyl | chloro | Y¹ is CH, Y² is N, |
| | chloro | | | Y³ is CH |
| P.1947 | X¹ is chloro, X² is CH, X³ is | chlorodifluoromethyl | cyano | Y¹ is CH, Y² is N, |
| | chloro | | | Y³ is CH |
| P.1948 | X¹ is chloro, X² is CH, X³ is | chlorodifluoromethyl | cyclopropyl | Y¹ is CH, Y² is N, |
| | chloro | | | Y³ is CH |
| P.1949 | X¹ is chloro, X² is CH, X³ is | chlorodifluoromethyl | ethyl | Y¹ is CH, Y² is N, |
| | chloro | | | Y³ is CH |
| P.1950 | X¹ is chloro, X² is CH, X³ is | chlorodifluoromethyl | fluoro | Y¹ is CH, Y² is N, |
| | chloro | | | Y³ is CH |
| P.1951 | X¹ is chloro, X² is CH, X³ is | chlorodifluoromethyl | hydrogen | Y¹ is CH, Y² is N, |
| | chloro | | | Y³ is CH |
| P.1952 | X¹ is chloro, X² is CH, X³ is | chlorodifluoromethyl | methoxy | Y¹ is CH, Y² is N, |
| | chloro | | | Y³ is CH |
| P.1953 | X¹ is chloro, X² is CH, X³ is | chlorodifluoromethyl | methyl | Y¹ is CH, Y² is N, |
| | chloro | | | Y³ is CH |
| P.1954 | X¹ is chloro, X² is CH, X³ is | chlorodifluoromethyl | nitro | Y¹ is CH, Y² is N, |
| | chloro | | | Y³ is CH |
| P.1955 | X¹ is chloro, X² is CH, X³ is | chlorodifluoromethyl | trifluoromethoxy | Y¹ is CH, Y² is N, |
| | chloro | | | Y³ is CH |
| P.1956 | X¹ is chloro, X² is CH, X³ is | chlorodifluoromethyl | trifluoromethyl | Y¹ is CH, Y² is N, |
| | chloro | | | Y³ is CH |
| P.1957 | X¹ is chloro, X² is CH, X³ is | difluoromethyl | bromo | Y¹ is CH, Y² is N, |
| | chloro | | | Y³ is CH |
| P.1958 | X¹ is chloro, X² is CH, X³ is | difluoromethyl | chloro | Y¹ is CH, Y² is N, |
| | chloro | | | Y³ is CH |
| P.1959 | X¹ is chloro, X² is CH, X³ is | difluoromethyl | cyano | Y¹ is CH, Y² is N, |
| | chloro | | | Y³ is CH |
| P.1960 | X¹ is chloro, X² is CH, X³ is | difluoromethyl | cyclopropyl | Y¹ is CH, Y² is N, |
| | chloro | | | Y³ is CH |
| P.1961 | X¹ is chloro, X² is CH, X³ is | difluoromethyl | ethyl | Y¹ is CH, Y² is N, |
| | chloro | | | Y³ is CH |
| P.1962 | X¹ is chloro, X² is CH, X³ is | difluoromethyl | fluoro | Y¹ is CH, Y² is N, |
| | chloro | | | Y³ is CH |
| P.1963 | X¹ is chloro, X² is CH, X³ is | difluoromethyl | hydrogen | Y¹ is CH, Y² is N, |
| | chloro | | | Y³ is CH |
| P.1964 | X¹ is chloro, X² is CH, X³ is | difluoromethyl | methoxy | Y¹ is CH, Y² is N, |
| | chloro | | | Y³ is CH |
| P.1965 | X¹ is chloro, X² is CH, X³ is | difluoromethyl | methyl | Y¹ is CH, Y² is N, |
| | chloro | | | Y³ is CH |
| P.1966 | X¹ is chloro, X² is CH, X³ is | difluoromethyl | nitro | Y¹ is CH, Y² is N, |
| | chloro | | | Y³ is CH |
| P.1967 | X¹ is chloro, X² is CH, X³ is | difluoromethyl | trifluoromethoxy | Y¹ is CH, Y² is N, |
| | chloro | | | Y³ is CH |
| P.1968 | X¹ is chloro, X² is CH, X³ is | difluoromethyl | trifluoromethyl | Y¹ is CH, Y² is N, |
| | chloro | | | Y³ is CH |
| P.1969 | X¹ is chloro, X² is CH, X³ is | trifluoromethyl | bromo | Y¹ is CH, Y² is N, |
| | chloro | | | Y³ is CH |
| P.1970 | X¹ is chloro, X² is CH, X³ is | trifluoromethyl | chloro | Y¹ is CH, Y² is N, |
| | chloro | | | Y³ is CH |
| P.1971 | X¹ is chloro, X² is CH, X³ is | trifluoromethyl | cyano | Y¹ is CH, Y² is N, |
| | chloro | | | Y³ is CH |
| P.1972 | X¹ is chloro, X² is CH, X³ is | trifluoromethyl | cyclopropyl | Y¹ is CH, Y² is N, |
| | chloro | | | Y³ is CH |
| P.1973 | X¹ is chloro, X² is CH, X³ is | trifluoromethyl | ethyl | Y¹ is CH, Y² is N, |
| | chloro | | | Y³ is CH |
| P.1974 | X¹ is chloro, X² is CH, X³ is | trifluoromethyl | fluoro | Y¹ is CH, Y² is N, |
| | chloro | | | Y³ is CH |
| P.1975 | X¹ is chloro, X² is CH, X³ is | trifluoromethyl | hydrogen | Y¹ is CH, Y² is N, |
| | chloro | | | Y³ is CH |
| P.1976 | X¹ is chloro, X² is CH, X³ is | trifluoromethyl | methoxy | Y¹ is CH, Y² is N, |
| | chloro | | | Y³ is CH |
| P.1977 | X¹ is chloro, X² is CH, X³ is | trifluoromethyl | methyl | Y¹ is CH, Y² is N, |
| | chloro | | | Y³ is CH |
| P.1978 | X¹ is chloro, X² is CH, X³ is | trifluoromethyl | nitro | Y¹ is CH, Y² is N, |
| | chloro | | | Y³ is CH |
| P.1979 | X¹ is chloro, X² is CH, X³ is | trifluoromethyl | trifluoromethoxy | Y¹ is CH, Y² is N, |
| | chloro | | | Y³ is CH |
| P.1980 | X¹ is chloro, X² is CH, X³ is | trifluoromethyl | trifluoromethyl | Y¹ is CH, Y² is N, |
| | chloro | | | Y³ is CH |
| P.1981 | X¹ is chloro, X² is C—F, X³ is | chlorodifluoromethyl | bromo | Y¹ is CH, Y² is N, |
| | hydrogen | | | Y³ is CH |
| P.1982 | X¹ is chloro, X² is C—F, X³ is | chlorodifluoromethyl | chloro | Y¹ is CH, Y² is N, |
| | hydrogen | | | Y³ is CH |
| P.1983 | X¹ is chloro, X² is C—F, X³ is | chlorodifluoromethyl | cyano | Y¹ is CH, Y² is N, |
| | hydrogen | | | Y³ is CH |
| P.1984 | X¹ is chloro, X² is C—F, X³ is | chlorodifluoromethyl | cyclopropyl | Y¹ is CH, Y² is N, |
| | hydrogen | | | Y³ is CH |
| P.1985 | X¹ is chloro, X² is C—F, X³ is | chlorodifluoromethyl | ethyl | Y¹ is CH, Y² is N, |
| | hydrogen | | | Y³ is CH |
| P.1986 | X¹ is chloro, X² is C—F, X³ is | chlorodifluoromethyl | fluoro | Y¹ is CH, Y² is N, |
| | hydrogen | | | Y³ is CH |
| P.1987 | X¹ is chloro, X² is C—F, X³ is | chlorodifluoromethyl | hydrogen | Y¹ is CH, Y² is N, |
| | hydrogen | | | Y³ is CH |
| P.1988 | X¹ is chloro, X² is C—F, X³ is | chlorodifluoromethyl | methoxy | Y¹ is CH, Y² is N, |
| | hydrogen | | | Y³ is CH |
| P.1989 | X¹ is chloro, X² is C—F, X³ is | chlorodifluoromethyl | methyl | Y¹ is CH, Y² is N, |
| | hydrogen | | | Y³ is CH |
| P.1990 | X¹ is chloro, X² is C—F, X³ is | chlorodifluoromethyl | nitro | Y¹ is CH, Y² is N, |
| | hydrogen | | | Y³ is CH |
| P.1991 | X¹ is chloro, X² is C—F, X³ is | chlorodifluoromethyl | trifluoromethoxy | Y¹ is CH, Y² is N, |
| | hydrogen | | | Y³ is CH |
| P.1992 | X¹ is chloro, X² is C—F, X³ is | chlorodifluoromethyl | trifluoromethyl | Y¹ is CH, Y² is N, |
| | hydrogen | | | Y³ is CH |
| P.1993 | X¹ is chloro, X² is C—F, X³ is | difluoromethyl | bromo | Y¹ is CH, Y² is N, |
| | hydrogen | | | Y³ is CH |
| P.1994 | X¹ is chloro, X² is C—F, X³ is | difluoromethyl | chloro | Y¹ is CH, Y² is N, |
| | hydrogen | | | Y³ is CH |
| P.1995 | X¹ is chloro, X² is C—F, X³ is | difluoromethyl | cyano | Y¹ is CH, Y² is N, |
| | hydrogen | | | Y³ is CH |
| P.1996 | X¹ is chloro, X² is C—F, X³ is | difluoromethyl | cyclopropyl | Y¹ is CH, Y² is N, |
| | hydrogen | | | Y³ is CH |
| P.1997 | X¹ is chloro, X² is C—F, X³ is | difluoromethyl | ethyl | Y¹ is CH, Y² is N, |
| | hydrogen | | | Y³ is CH |
| P.1998 | X¹ is chloro, X² is C—F, X³ is | difluoromethyl | fluoro | Y¹ is CH, Y² is N, |
| | hydrogen | | | Y³ is CH |
| P.1999 | X¹ is chloro, X² is C—F, X³ is | difluoromethyl | hydrogen | Y¹ is CH, Y² is N, |
| | hydrogen | | | Y³ is CH |
| P.2000 | X¹ is chloro, X² is C—F, X³ is | difluoromethyl | methoxy | Y¹ is CH, Y² is N, |
| | hydrogen | | | Y³ is CH |
| P.2001 | X¹ is chloro, X² is C—F, X³ is | difluoromethyl | methyl | Y¹ is CH, Y² is N, |
| | hydrogen | | | Y³ is CH |
| P.2002 | X¹ is chloro, X² is C—F, X³ is | difluoromethyl | nitro | Y¹ is CH, Y² is N, |
| | hydrogen | | | Y³ is CH |
| P.2003 | X¹ is chloro, X² is C—F, X³ is | difluoromethyl | trifluoromethoxy | Y¹ is CH, Y² is N, |
| | hydrogen | | | Y³ is CH |
| P.2004 | X¹ is chloro, X² is C—F, X³ is | difluoromethyl | trifluoromethyl | Y¹ is CH, Y² is N, |
| | hydrogen | | | Y³ is CH |
| P.2005 | X¹ is chloro, X² is C—F, X³ is | trifluoromethyl | bromo | Y¹ is CH, Y² is N, |
| | hydrogen | | | Y³ is CH |
| P.2006 | X¹ is chloro, X² is C—F, X³ is | trifluoromethyl | chloro | Y¹ is CH, Y² is N, |
| | hydrogen | | | Y³ is CH |
| P.2007 | X¹ is chloro, X² is C—F, X³ is | trifluoromethyl | cyano | Y¹ is CH, Y² is N, |
| | hydrogen | | | Y³ is CH |
| P.2008 | X¹ is chloro, X² is C—F, X³ is | trifluoromethyl | cyclopropyl | Y¹ is CH, Y² is N, |
| | hydrogen | | | Y³ is CH |
| P.2009 | X¹ is chloro, X² is C—F, X³ is | trifluoromethyl | ethyl | Y¹ is CH, Y² is N, |
| | hydrogen | | | Y³ is CH |
| P.2010 | X¹ is chloro, X² is C—F, X³ is | trifluoromethyl | fluoro | Y¹ is CH, Y² is N, |
| | hydrogen | | | Y³ is CH |
| P.2011 | X¹ is chloro, X² is C—F, X³ is | trifluoromethyl | hydrogen | Y¹ is CH, Y² is N, |
| | hydrogen | | | Y³ is CH |
| P.2012 | X¹ is chloro, X² is C—F, X³ is | trifluoromethyl | methoxy | Y¹ is CH, Y² is N, |
| | hydrogen | | | Y³ is CH |
| P.2013 | X¹ is chloro, X² is C—F, X³ is | trifluoromethyl | methyl | Y¹ is CH, Y² is N, |
| | hydrogen | | | Y³ is CH |
| P.2014 | X¹ is chloro, X² is C—F, X³ is | trifluoromethyl | nitro | Y¹ is CH, Y² is N, |
| | hydrogen | | | Y³ is CH |
| P.2015 | X¹ is chloro, X² is C—F, X³ is | trifluoromethyl | trifluoromethoxy | Y¹ is CH, Y² is N, |
| | hydrogen | | | Y³ is CH |
| P.2016 | X¹ is chloro, X² is C—F, X³ is | trifluoromethyl | trifluoromethyl | Y¹ is CH, Y² is N, |
| | hydrogen | | | Y³ is CH |
| P.2017 | X¹ is fluoro, X² is C—Cl, X³ | chlorodifluoromethyl | bromo | Y¹ is CH, Y² is N, |
| | is hydrogen | | | Y³ is CH |
| P.2018 | X¹ is fluoro, X² is C—Cl, X³ | chlorodifluoromethyl | chloro | Y¹ is CH, Y² is N, |
| | is hydrogen | | | Y³ is CH |
| P.2019 | X¹ is fluoro, X² is C—Cl, X³ | chlorodifluoromethyl | cyano | Y¹ is CH, Y² is N, |
| | is hydrogen | | | Y³ is CH |
| P.2020 | X¹ is fluoro, X² is C—Cl, X³ | chlorodifluoromethyl | cyclopropyl | Y¹ is CH, Y² is N, |
| | is hydrogen | | | Y³ is CH |
| P.2021 | X¹ is fluoro, X² is C—Cl, X³ | chlorodifluoromethyl | ethyl | Y¹ is CH, Y² is N, |
| | is hydrogen | | | Y³ is CH |
| P.2022 | X¹ is fluoro, X² is C—Cl, X³ | chlorodifluoromethyl | fluoro | Y¹ is CH, Y² is N, |
| | is hydrogen | | | Y³ is CH |
| P.2023 | X¹ is fluoro, X² is C—Cl, X³ | chlorodifluoromethyl | hydrogen | Y¹ is CH, Y² is N, |
| | is hydrogen | | | Y³ is CH |
| P.2024 | X¹ is fluoro, X² is C—Cl, X³ | chlorodifluoromethyl | methoxy | Y¹ is CH, Y² is N, |
| | is hydrogen | | | Y³ is CH |
| P.2025 | X¹ is fluoro, X² is C—Cl, X³ | chlorodifluoromethyl | methyl | Y¹ is CH, Y² is N, |
| | is hydrogen | | | Y³ is CH |
| P.2026 | X¹ is fluoro, X² is C—Cl, X³ | chlorodifluoromethyl | nitro | Y¹ is CH, Y² is N, |
| | is hydrogen | | | Y³ is CH |
| P.2027 | X¹ is fluoro, X² is C—Cl, X³ | chlorodifluoromethyl | trifluoromethoxy | Y¹ is CH, Y² is N, |
| | is hydrogen | | | Y³ is CH |
| P.2028 | X¹ is fluoro, X² is C—Cl, X³ | chlorodifluoromethyl | trifluoromethyl | Y¹ is CH, Y² is N, |
| | is hydrogen | | | Y³ is CH |
| P.2029 | X¹ is fluoro, X² is C—Cl, X³ | difluoromethyl | bromo | Y¹ is CH, Y² is N, |
| | is hydrogen | | | Y³ is CH |
| P.2030 | X¹ is fluoro, X² is C—Cl, X³ | difluoromethyl | chloro | Y¹ is CH, Y² is N, |
| | is hydrogen | | | Y³ is CH |
| P.2031 | X¹ is fluoro, X² is C—Cl, X³ | difluoromethyl | cyano | Y¹ is CH, Y² is N, |
| | is hydrogen | | | Y³ is CH |
| P.2032 | X¹ is fluoro, X² is C—Cl, X³ | difluoromethyl | cyclopropyl | Y¹ is CH, Y² is N, |
| | is hydrogen | | | Y³ is CH |
| P.2033 | X¹ is fluoro, X² is C—Cl, X³ | difluoromethyl | ethyl | Y¹ is CH, Y² is N, |
| | is hydrogen | | | Y³ is CH |
| P.2034 | X¹ is fluoro, X² is C—Cl, X³ | difluoromethyl | fluoro | Y¹ is CH, Y² is N, |
| | is hydrogen | | | Y³ is CH |
| P.2035 | X¹ is fluoro, X² is C—Cl, X³ | difluoromethyl | hydrogen | Y¹ is CH, Y² is N, |
| | is hydrogen | | | Y³ is CH |
| P.2036 | X¹ is fluoro, X² is C—Cl, X³ | difluoromethyl | methoxy | Y¹ is CH, Y² is N, |
| | is hydrogen | | | Y³ is CH |
| P.2037 | X¹ is fluoro, X² is C—Cl, X³ | difluoromethyl | methyl | Y¹ is CH, Y² is N, |
| | is hydrogen | | | Y³ is CH |
| P.2038 | X¹ is fluoro, X² is C—Cl, X³ | difluoromethyl | nitro | Y¹ is CH, Y² is N, |
| | is hydrogen | | | Y³ is CH |
| P.2039 | X¹ is fluoro, X² is C—Cl, X³ | difluoromethyl | trifluoromethoxy | Y¹ is CH, Y² is N, |
| | is hydrogen | | | Y³ is CH |
| P.2040 | X¹ is fluoro, X² is C—Cl, X³ | difluoromethyl | trifluoromethyl | Y¹ is CH, Y² is N, |
| | is hydrogen | | | Y³ is CH |
| P.2041 | X¹ is fluoro, X² is C—Cl, X³ | trifluoromethyl | bromo | Y¹ is CH, Y² is N, |
| | is hydrogen | | | Y³ is CH |
| P.2042 | X¹ is fluoro, X² is C—Cl, X³ | trifluoromethyl | chloro | Y¹ is CH, Y² is N, |
| | is hydrogen | | | Y³ is CH |
| P.2043 | X¹ is fluoro, X² is C—Cl, X³ | trifluoromethyl | cyano | Y¹ is CH, Y² is N, |
| | is hydrogen | | | Y³ is CH |
| P.2044 | X¹ is fluoro, X² is C—Cl, X³ | trifluoromethyl | cyclopropyl | Y¹ is CH, Y² is N, |
| | is hydrogen | | | Y³ is CH |
| P.2045 | X¹ is fluoro, X² is C—Cl, X³ | trifluoromethyl | ethyl | Y¹ is CH, Y² is N, |
| | is hydrogen | | | Y³ is CH |
| P.2046 | X¹ is fluoro, X² is C—Cl, X³ | trifluoromethyl | fluoro | Y¹ is CH, Y² is N, |
| | is hydrogen | | | Y³ is CH |
| P.2047 | X¹ is fluoro, X² is C—Cl, X³ | trifluoromethyl | hydrogen | Y¹ is CH, Y² is N, |
| | is hydrogen | | | Y³ is CH |
| P.2048 | X¹ is fluoro, X² is C—Cl, X³ | trifluoromethyl | methoxy | Y¹ is CH, Y² is N, |
| | is hydrogen | | | Y³ is CH |
| P.2049 | X¹ is fluoro, X² is C—Cl, X³ | trifluoromethyl | methyl | Y¹ is CH, Y² is N, |
| | is hydrogen | | | Y³ is CH |
| P.2050 | X¹ is fluoro, X² is C—Cl, X³ | trifluoromethyl | nitro | Y¹ is CH, Y² is N, |
| | is hydrogen | | | Y³ is CH |
| P.2051 | X¹ is fluoro, X² is C—Cl, X³ | trifluoromethyl | trifluoromethoxy | Y¹ is CH, Y² is N, |
| | is hydrogen | | | Y³ is CH |
| P.2052 | X¹ is fluoro, X² is C—Cl, X³ | trifluoromethyl | trifluoromethyl | Y¹ is CH, Y² is N, |
| | is hydrogen | | | Y³ is CH |
| P.2053 | X¹ is chloro, X² is C—Cl, X³ | chlorodifluoromethyl | bromo | Y¹ is CH, Y² is N, |
| | is hydrogen | | | Y³ is CH |
| P.2054 | X¹ is chloro, X² is C—Cl, X³ | chlorodifluoromethyl | chloro | Y¹ is CH, Y² is N, |
| | is hydrogen | | | Y³ is CH |
| P.2055 | X¹ is chloro, X² is C—Cl, X³ | chlorodifluoromethyl | cyano | Y¹ is CH, Y² is N, |
| | is hydrogen | | | Y³ is CH |
| P.2056 | X¹ is chloro, X² is C—Cl, X³ | chlorodifluoromethyl | cyclopropyl | Y¹ is CH, Y² is N, |
| | is hydrogen | | | Y³ is CH |
| P.2057 | X¹ is chloro, X² is C—Cl, X³ | chlorodifluoromethyl | ethyl | Y¹ is CH, Y² is N, |
| | is hydrogen | | | Y³ is CH |
| P.2058 | X¹ is chloro, X² is C—Cl, X³ | chlorodifluoromethyl | fluoro | Y¹ is CH, Y² is N, |
| | is hydrogen | | | Y³ is CH |
| P.2059 | X¹ is chloro, X² is C—Cl, X³ | chlorodifluoromethyl | hydrogen | Y¹ is CH, Y² is N, |
| | is hydrogen | | | Y³ is CH |
| P.2060 | X¹ is chloro, X² is C—Cl, X³ | chlorodifluoromethyl | methoxy | Y¹ is CH, Y² is N, |
| | is hydrogen | | | Y³ is CH |
| P.2061 | X¹ is chloro, X² is C—Cl, X³ | chlorodifluoromethyl | methyl | Y¹ is CH, Y² is N, |
| | is hydrogen | | | Y³ is CH |
| P.2062 | X¹ is chloro, X² is C—Cl, X³ | chlorodifluoromethyl | nitro | Y¹ is CH, Y² is N, |
| | is hydrogen | | | Y³ is CH |
| P.2063 | X¹ is chloro, X² is C—Cl, X³ | chlorodifluoromethyl | trifluoromethoxy | Y¹ is CH, Y² is N, |
| | is hydrogen | | | Y³ is CH |
| P.2064 | X¹ is chloro, X² is C—Cl, X³ | chlorodifluoromethyl | trifluoromethyl | Y¹ is CH, Y² is N, |
| | is hydrogen | | | Y³ is CH |
| P.2065 | X¹ is chloro, X² is C—Cl, X³ | difluoromethyl | bromo | Y¹ is CH, Y² is N, |
| | is hydrogen | | | Y³ is CH |
| P.2066 | X¹ is chloro, X² is C—Cl, X³ | difluoromethyl | chloro | Y¹ is CH, Y² is N, |
| | is hydrogen | | | Y³ is CH |
| P.2067 | X¹ is chloro, X² is C—Cl, X³ | difluoromethyl | cyano | Y¹ is CH, Y² is N, |
| | is hydrogen | | | Y³ is CH |
| P.2068 | X¹ is chloro, X² is C—Cl, X³ | difluoromethyl | cyclopropyl | Y¹ is CH, Y² is N, |
| | is hydrogen | | | Y³ is CH |
| P.2069 | X¹ is chloro, X² is C—Cl, X³ | difluoromethyl | ethyl | Y¹ is CH, Y² is N, |
| | is hydrogen | | | Y³ is CH |
| P.2070 | X¹ is chloro, X² is C—Cl, X³ | difluoromethyl | fluoro | Y¹ is CH, Y² is N, |
| | is hydrogen | | | Y³ is CH |
| P.2071 | X¹ is chloro, X² is C—Cl, X³ | difluoromethyl | hydrogen | Y¹ is CH, Y² is N, |
| | is hydrogen | | | Y³ is CH |
| P.2072 | X¹ is chloro, X² is C—Cl, X³ | difluoromethyl | methoxy | Y¹ is CH, Y² is N, |
| | is hydrogen | | | Y³ is CH |
| P.2073 | X¹ is chloro, X² is C—Cl, X³ | difluoromethyl | methyl | Y¹ is CH, Y² is N, |
| | is hydrogen | | | Y³ is CH |
| P.2074 | X¹ is chloro, X² is C—Cl, X³ | difluoromethyl | nitro | Y¹ is CH, Y² is N, |
| | is hydrogen | | | Y³ is CH |
| P.2075 | X¹ is chloro, X² is C—Cl, X³ | difluoromethyl | trifluoromethoxy | Y¹ is CH, Y² is N, |
| | is hydrogen | | | Y³ is CH |
| P.2076 | X¹ is chloro, X² is C—Cl, X³ | difluoromethyl | trifluoromethyl | Y¹ is CH, Y² is N, |
| | is hydrogen | | | Y³ is CH |
| P.2077 | X¹ is chloro, X² is C—Cl, X³ | trifluoromethyl | bromo | Y¹ is CH, Y² is N, |
| | is hydrogen | | | Y³ is CH |
| P.2078 | X¹ is chloro, X² is C—Cl, X³ | trifluoromethyl | chloro | Y¹ is CH, Y² is N, |
| | is hydrogen | | | Y³ is CH |
| P.2079 | X¹ is chloro, X² is C—Cl, X³ | trifluoromethyl | cyano | Y¹ is CH, Y² is N, |
| | is hydrogen | | | Y³ is CH |
| P.2080 | X¹ is chloro, X² is C—Cl, X³ | trifluoromethyl | cyclopropyl | Y¹ is CH, Y² is N, |
| | is hydrogen | | | Y³ is CH |
| P.2081 | X¹ is chloro, X² is C—Cl, X³ | trifluoromethyl | ethyl | Y¹ is CH, Y² is N, |
| | is hydrogen | | | Y³ is CH |
| P.2082 | X¹ is chloro, X² is C—Cl, X³ | trifluoromethyl | fluoro | Y¹ is CH, Y² is N, |
| | is hydrogen | | | Y³ is CH |
| P.2083 | X¹ is chloro, X² is C—Cl, X³ | trifluoromethyl | hydrogen | Y¹ is CH, Y² is N, |
| | is hydrogen | | | Y³ is CH |
| P.2084 | X¹ is chloro, X² is C—Cl, X³ | trifluoromethyl | methoxy | Y¹ is CH, Y² is N, |
| | is hydrogen | | | Y³ is CH |
| P.2085 | X¹ is chloro, X² is C—Cl, X³ | trifluoromethyl | methyl | Y¹ is CH, Y² is N, |
| | is hydrogen | | | Y³ is CH |
| P.2086 | X¹ is chloro, X² is C—Cl, X³ | trifluoromethyl | nitro | Y¹ is CH, Y² is N, |
| | is hydrogen | | | Y³ is CH |
| P.2087 | X¹ is chloro, X² is C—Cl, X³ | trifluoromethyl | trifluoromethoxy | Y¹ is CH, Y² is N, |
| | is hydrogen | | | Y³ is CH |
| P.2088 | X¹ is chloro, X² is C—Cl, X³ | trifluoromethyl | trifluoromethyl | Y¹ is CH, Y² is N, |
| | is hydrogen | | | Y³ is CH |
| P.2089 | X¹ is chloro, X² is C—Br, X³ | chlorodifluoromethyl | bromo | Y¹ is CH, Y² is N, |
| | is chloro | | | Y³ is CH |
| P.2090 | X¹ is chloro, X² is C—Br, X³ | chlorodifluoromethyl | chloro | Y¹ is CH, Y² is N, |
| | is chloro | | | Y³ is CH |
| P.2091 | X¹ is chloro, X² is C—Br, X³ | chlorodifluoromethyl | cyano | Y¹ is CH, Y² is N, |
| | is chloro | | | Y³ is CH |
| P.2092 | X¹ is chloro, X² is C—Br, X³ | chlorodifluoromethyl | cyclopropyl | Y¹ is CH, Y² is N, |
| | is chloro | | | Y³ is CH |
| P.2093 | X¹ is chloro, X² is C—Br, X³ | chlorodifluoromethyl | ethyl | Y¹ is CH, Y² is N, |
| | is chloro | | | Y³ is CH |
| P.2094 | X¹ is chloro, X² is C—Br, X³ | chlorodifluoromethyl | fluoro | Y¹ is CH, Y² is N, |
| | is chloro | | | Y³ is CH |
| P.2095 | X¹ is chloro, X² is C—Br, X³ | chlorodifluoromethyl | hydrogen | Y¹ is CH, Y² is N, |
| | is chloro | | | Y³ is CH |
| P.2096 | X¹ is chloro, X² is C—Br, X³ | chlorodifluoromethyl | methoxy | Y¹ is CH, Y² is N, |
| | is chloro | | | Y³ is CH |
| P.2097 | X¹ is chloro, X² is C—Br, X³ | chlorodifluoromethyl | methyl | Y¹ is CH, Y² is N, |
| | is chloro | | | Y³ is CH |
| P.2098 | X¹ is chloro, X² is C—Br, X³ | chlorodifluoromethyl | nitro | Y¹ is CH, Y² is N, |
| | is chloro | | | Y³ is CH |
| P.2099 | X¹ is chloro, X² is C—Br, X³ | chlorodifluoromethyl | trifluoromethoxy | Y¹ is CH, Y² is N, |
| | is chloro | | | Y³ is CH |
| P.2100 | X¹ is chloro, X² is C—Br, X³ | chlorodifluoromethyl | trifluoromethyl | Y¹ is CH, Y² is N, |
| | is chloro | | | Y³ is CH |
| P.2101 | X¹ is chloro, X² is C—Br, X³ | difluoromethyl | bromo | Y¹ is CH, Y² is N, |
| | is chloro | | | Y³ is CH |
| P.2102 | X¹ is chloro, X² is C—Br, X³ | difluoromethyl | chloro | Y¹ is CH, Y² is N, |
| | is chloro | | | Y³ is CH |
| P.2103 | X¹ is chloro, X² is C—Br, X³ | difluoromethyl | cyano | Y¹ is CH, Y² is N, |
| | is chloro | | | Y³ is CH |
| P.2104 | X¹ is chloro, X² is C—Br, X³ | difluoromethyl | cyclopropyl | Y¹ is CH, Y² is N, |
| | is chloro | | | Y³ is CH |
| P.2105 | X¹ is chloro, X² is C—Br, X³ | difluoromethyl | ethyl | Y¹ is CH, Y² is N, |
| | is chloro | | | Y³ is CH |
| P.2106 | X¹ is chloro, X² is C—Br, X³ | difluoromethyl | fluoro | Y¹ is CH, Y² is N, |
| | is chloro | | | Y³ is CH |
| P.2107 | X¹ is chloro, X² is C—Br, X³ | difluoromethyl | hydrogen | Y¹ is CH, Y² is N, |
| | is chloro | | | Y³ is CH |
| P.2108 | X¹ is chloro, X² is C—Br, X³ | difluoromethyl | methoxy | Y¹ is CH, Y² is N, |
| | is chloro | | | Y³ is CH |
| P.2109 | X¹ is chloro, X² is C—Br, X³ | difluoromethyl | methyl | Y¹ is CH, Y² is N, |
| | is chloro | | | Y³ is CH |
| P.2110 | X¹ is chloro, X² is C—Br, X³ | difluoromethyl | nitro | Y¹ is CH, Y² is N, |
| | is chloro | | | Y³ is CH |
| P.2111 | X¹ is chloro, X² is C—Br, X³ | difluoromethyl | trifluoromethoxy | Y¹ is CH, Y² is N, |
| | is chloro | | | Y³ is CH |
| P.2112 | X¹ is chloro, X² is C—Br, X³ | difluoromethyl | trifluoromethyl | Y¹ is CH, Y² is N, |
| | is chloro | | | Y³ is CH |
| P.2113 | X¹ is chloro, X² is C—Br, X³ | trifluoromethyl | bromo | Y¹ is CH, Y² is N, |
| | is chloro | | | Y³ is CH |
| P.2114 | X¹ is chloro, X² is C—Br, X³ | trifluoromethyl | chloro | Y¹ is CH, Y² is N, |
| | is chloro | | | Y³ is CH |
| P.2115 | X¹ is chloro, X² is C—Br, X³ | trifluoromethyl | cyano | Y¹ is CH, Y² is N, |
| | is chloro | | | Y³ is CH |
| P.2116 | X¹ is chloro, X² is C—Br, X³ | trifluoromethyl | cyclopropyl | Y¹ is CH, Y² is N, |
| | is chloro | | | Y³ is CH |
| P.2117 | X¹ is chloro, X² is C—Br, X³ | trifluoromethyl | ethyl | Y¹ is CH, Y² is N, |
| | is chloro | | | Y³ is CH |
| P.2118 | X¹ is chloro, X² is C—Br, X³ | trifluoromethyl | fluoro | Y¹ is CH, Y² is N, |
| | is chloro | | | Y³ is CH |
| P.2119 | X¹ is chloro, X² is C—Br, X³ | trifluoromethyl | hydrogen | Y¹ is CH, Y² is N, |
| | is chloro | | | Y³ is CH |
| P.2120 | X¹ is chloro, X² is C—Br, X³ | trifluoromethyl | methoxy | Y¹ is CH, Y² is N, |
| | is chloro | | | Y³ is CH |
| P.2121 | X¹ is chloro, X² is C—Br, X³ | trifluoromethyl | methyl | Y¹ is CH, Y² is N, |
| | is chloro | | | Y³ is CH |
| P.2122 | X¹ is chloro, X² is C—Br, X³ | trifluoromethyl | nitro | Y¹ is CH, Y² is N, |
| | is chloro | | | Y³ is CH |
| P.2123 | X¹ is chloro, X² is C—Br, X³ | trifluoromethyl | trifluoromethoxy | Y¹ is CH, Y² is N, |
| | is chloro | | | Y³ is CH |
| P.2124 | X¹ is chloro, X² is C—Br, X³ | trifluoromethyl | trifluoromethyl | Y¹ is CH, Y² is N, |
| | is chloro | | | Y³ is CH |
| P.2125 | X¹ is chloro, X² is C—F, X³ is | chlorodifluoromethyl | bromo | Y¹ is CH, Y² is N, |
| | chloro | | | Y³ is CH |
| P.2126 | X¹ is chloro, X² is C—F, X³ is | chlorodifluoromethyl | chloro | Y¹ is CH, Y² is N, |
| | chloro | | | Y³ is CH |
| P.2127 | X¹ is chloro, X² is C—F, X³ is | chlorodifluoromethyl | cyano | Y¹ is CH, Y² is N, |
| | chloro | | | Y³ is CH |
| P.2128 | X¹ is chloro, X² is C—F, X³ is | chlorodifluoromethyl | cyclopropyl | Y¹ is CH, Y² is N, |
| | chloro | | | Y³ is CH |
| P.2129 | X¹ is chloro, X² is C—F, X³ is | chlorodifluoromethyl | ethyl | Y¹ is CH, Y² is N, |
| | chloro | | | Y³ is CH |
| P.2130 | X¹ is chloro, X² is C—F, X³ is | chlorodifluoromethyl | fluoro | Y¹ is CH, Y² is N, |
| | chloro | | | Y³ is CH |
| P.2131 | X¹ is chloro, X² is C—F, X³ is | chlorodifluoromethyl | hydrogen | Y¹ is CH, Y² is N, |
| | chloro | | | Y³ is CH |
| P.2132 | X¹ is chloro, X² is C—F, X³ is | chlorodifluoromethyl | methoxy | Y¹ is CH, Y² is N, |
| | chloro | | | Y³ is CH |
| P.2133 | X¹ is chloro, X² is C—F, X³ is | chlorodifluoromethyl | methyl | Y¹ is CH, Y² is N, |
| | chloro | | | Y³ is CH |
| P.2134 | X¹ is chloro, X² is C—F, X³ is | chlorodifluoromethyl | nitro | Y¹ is CH, Y² is N, |
| | chloro | | | Y³ is CH |
| P.2135 | X¹ is chloro, X² is C—F, X³ is | chlorodifluoromethyl | trifluoromethoxy | Y¹ is CH, Y² is N, |
| | chloro | | | Y³ is CH |
| P.2136 | X¹ is chloro, X² is C—F, X³ is | chlorodifluoromethyl | trifluoromethyl | Y¹ is CH, Y² is N, |
| | chloro | | | Y³ is CH |
| P.2137 | X¹ is chloro, X² is C—F, X³ is | difluoromethyl | bromo | Y¹ is CH, Y² is N, |
| | chloro | | | Y³ is CH |
| P.2138 | X¹ is chloro, X² is C—F, X³ is | difluoromethyl | chloro | Y¹ is CH, Y² is N, |
| | chloro | | | Y³ is CH |
| P.2139 | X¹ is chloro, X² is C—F, X³ is | difluoromethyl | cyano | Y¹ is CH, Y² is N, |
| | chloro | | | Y³ is CH |
| P.2140 | X¹ is chloro, X² is C—F, X³ is | difluoromethyl | cyclopropyl | Y¹ is CH, Y² is N, |
| | chloro | | | Y³ is CH |
| P.2141 | X¹ is chloro, X² is C—F, X³ is | difluoromethyl | ethyl | Y¹ is CH, Y² is N, |
| | chloro | | | Y³ is CH |
| P.2142 | X¹ is chloro, X² is C—F, X³ is | difluoromethyl | fluoro | Y¹ is CH, Y² is N, |
| | chloro | | | Y³ is CH |
| P.2143 | X¹ is chloro, X² is C—F, X³ is | difluoromethyl | hydrogen | Y¹ is CH, Y² is N, |
| | chloro | | | Y³ is CH |
| P.2144 | X¹ is chloro, X² is C—F, X³ is | difluoromethyl | methoxy | Y¹ is CH, Y² is N, |
| | chloro | | | Y³ is CH |
| P.2145 | X¹ is chloro, X² is C—F, X³ is | difluoromethyl | methyl | Y¹ is CH, Y² is N, |
| | chloro | | | Y³ is CH |
| P.2146 | X¹ is chloro, X² is C—F, X³ is | difluoromethyl | nitro | Y¹ is CH, Y² is N, |
| | chloro | | | Y³ is CH |
| P.2147 | X¹ is chloro, X² is C—F, X³ is | difluoromethyl | trifluoromethoxy | Y¹ is CH, Y² is N, |
| | chloro | | | Y³ is CH |
| P.2148 | X¹ is chloro, X² is C—F, X³ is | difluoromethyl | trifluoromethyl | Y¹ is CH, Y² is N, |
| | chloro | | | Y³ is CH |
| P.2149 | X¹ is chloro, X² is C—F, X³ is | trifluoromethyl | bromo | Y¹ is CH, Y² is N, |
| | chloro | | | Y³ is CH |
| P.2150 | X¹ is chloro, X² is C—F, X³ is | trifluoromethyl | chloro | Y¹ is CH, Y² is N, |
| | chloro | | | Y³ is CH |
| P.2151 | X¹ is chloro, X² is C—F, X³ is | trifluoromethyl | cyano | Y¹ is CH, Y² is N, |
| | chloro | | | Y³ is CH |
| P.2152 | X¹ is chloro, X² is C—F, X³ is | trifluoromethyl | cyclopropyl | Y¹ is CH, Y² is N, |
| | chloro | | | Y³ is CH |
| P.2153 | X¹ is chloro, X² is C—F, X³ is | trifluoromethyl | ethyl | Y¹ is CH, Y² is N, |
| | chloro | | | Y³ is CH |
| P.2154 | X¹ is chloro, X² is C—F, X³ is | trifluoromethyl | fluoro | Y¹ is CH, Y² is N, |
| | chloro | | | Y³ is CH |
| P.2155 | X¹ is chloro, X² is C—F, X³ is | trifluoromethyl | hydrogen | Y¹ is CH, Y² is N, |
| | chloro | | | Y³ is CH |
| P.2156 | X¹ is chloro, X² is C—F, X³ is | trifluoromethyl | methoxy | Y¹ is CH, Y² is N, |
| | chloro | | | Y³ is CH |
| P.2157 | X¹ is chloro, X² is C—F, X³ is | trifluoromethyl | methyl | Y¹ is CH, Y² is N, |
| | chloro | | | Y³ is CH |
| P.2158 | X¹ is chloro, X² is C—F, X³ is | trifluoromethyl | nitro | Y¹ is CH, Y² is N, |
| | chloro | | | Y³ is CH |
| P.2159 | X¹ is chloro, X² is C—F, X³ is | trifluoromethyl | trifluoromethoxy | Y¹ is CH, Y² is N, |
| | chloro | | | Y³ is CH |
| P.2160 | X¹ is chloro, X² is C—F, X³ is | trifluoromethyl | trifluoromethyl | Y¹ is CH, Y² is N, |
| | chloro | | | Y³ is CH |
| P.2161 | X¹ is chloro, X² is C—Cl, X³ | chlorodifluoromethyl | bromo | Y¹ is CH, Y² is N, |
| | is chloro | | | Y³ is CH |
| P.2162 | X¹ is chloro, X² is C—Cl, X³ | chlorodifluoromethyl | chloro | Y¹ is CH, Y² is N, |
| | is chloro | | | Y³ is CH |
| P.2163 | X¹ is chloro, X² is C—Cl, X³ | chlorodifluoromethyl | cyano | Y¹ is CH, Y² is N, |
| | is chloro | | | Y³ is CH |
| P.2164 | X¹ is chloro, X² is C—Cl, X³ | chlorodifluoromethyl | cyclopropyl | Y¹ is CH, Y² is N, |
| | is chloro | | | Y³ is CH |
| P.2165 | X¹ is chloro, X² is C—Cl, X³ | chlorodifluoromethyl | ethyl | Y¹ is CH, Y² is N, |
| | is chloro | | | Y³ is CH |
| P.2166 | X¹ is chloro, X² is C—Cl, X³ | chlorodifluoromethyl | fluoro | Y¹ is CH, Y² is N, |
| | is chloro | | | Y³ is CH |
| P.2167 | X¹ is chloro, X² is C—Cl, X³ | chlorodifluoromethyl | hydrogen | Y¹ is CH, Y² is N, |
| | is chloro | | | Y³ is CH |
| P.2168 | X¹ is chloro, X² is C—Cl, X³ | chlorodifluoromethyl | methoxy | Y¹ is CH, Y² is N, |
| | is chloro | | | Y³ is CH |
| P.2169 | X¹ is chloro, X² is C—Cl, X³ | chlorodifluoromethyl | methyl | Y¹ is CH, Y² is N, |
| | is chloro | | | Y³ is CH |
| P.2170 | X¹ is chloro, X² is C—Cl, X³ | chlorodifluoromethyl | nitro | Y¹ is CH, Y² is N, |
| | is chloro | | | Y³ is CH |
| P.2171 | X¹ is chloro, X² is C—Cl, X³ | chlorodifluoromethyl | trifluoromethoxy | Y¹ is CH, Y² is N, |
| | is chloro | | | Y³ is CH |
| P.2172 | X¹ is chloro, X² is C—Cl, X³ | chlorodifluoromethyl | trifluoromethyl | Y¹ is CH, Y² is N, |
| | is chloro | | | Y³ is CH |
| P.2173 | X¹ is chloro, X² is C—Cl, X³ | difluoromethyl | bromo | Y¹ is CH, Y² is N, |
| | is chloro | | | Y³ is CH |
| P.2174 | X¹ is chloro, X² is C—Cl, X³ | difluoromethyl | chloro | Y¹ is CH, Y² is N, |
| | is chloro | | | Y³ is CH |
| P.2175 | X¹ is chloro, X² is C—Cl, X³ | difluoromethyl | cyano | Y¹ is CH, Y² is N, |
| | is chloro | | | Y³ is CH |
| P.2176 | X¹ is chloro, X² is C—Cl, X³ | difluoromethyl | cyclopropyl | Y¹ is CH, Y² is N, |
| | is chloro | | | Y³ is CH |
| P.2177 | X¹ is chloro, X² is C—Cl, X³ | difluoromethyl | ethyl | Y¹ is CH, Y² is N, |
| | is chloro | | | Y³ is CH |
| P.2178 | X¹ is chloro, X² is C—Cl, X³ | difluoromethyl | fluoro | Y¹ is CH, Y² is N, |
| | is chloro | | | Y³ is CH |
| P.2179 | X¹ is chloro, X² is C—Cl, X³ | difluoromethyl | hydrogen | Y¹ is CH, Y² is N, |
| | is chloro | | | Y³ is CH |
| P.2180 | X¹ is chloro, X² is C—Cl, X³ | difluoromethyl | methoxy | Y¹ is CH, Y² is N, |
| | is chloro | | | Y³ is CH |
| P.2181 | X¹ is chloro, X² is C—Cl, X³ | difluoromethyl | methyl | Y¹ is CH, Y² is N, |
| | is chloro | | | Y³ is CH |
| P.2182 | X¹ is chloro, X² is C—Cl, X³ | difluoromethyl | nitro | Y¹ is CH, Y² is N, |
| | is chloro | | | Y³ is CH |
| P.2183 | X¹ is chloro, X² is C—Cl, X³ | difluoromethyl | trifluoromethoxy | Y¹ is CH, Y² is N, |
| | is chloro | | | Y³ is CH |
| P.2184 | X¹ is chloro, X² is C—Cl, X³ | difluoromethyl | trifluoromethyl | Y¹ is CH, Y² is N, |
| | is chloro | | | Y³ is CH |
| P.2185 | X¹ is chloro, X² is C—Cl, X³ | trifluoromethyl | bromo | Y¹ is CH, Y² is N, |
| | is chloro | | | Y³ is CH |
| P.2186 | X¹ is chloro, X² is C—Cl, X³ | trifluoromethyl | chloro | Y¹ is CH, Y² is N, |
| | is chloro | | | Y³ is CH |
| P.2187 | X¹ is chloro, X² is C—Cl, X³ | trifluoromethyl | cyano | Y¹ is CH, Y² is N, |
| | is chloro | | | Y³ is CH |
| P.2188 | X¹ is chloro, X² is C—Cl, X³ | trifluoromethyl | cyclopropyl | Y¹ is CH, Y² is N, |
| | is chloro | | | Y³ is CH |
| P.2189 | X¹ is chloro, X² is C—Cl, X³ | trifluoromethyl | ethyl | Y¹ is CH, Y² is N, |
| | is chloro | | | Y³ is CH |
| P.2190 | X¹ is chloro, X² is C—Cl, X³ | trifluoromethyl | fluoro | Y¹ is CH, Y² is N, |
| | is chloro | | | Y³ is CH |
| P.2191 | X¹ is chloro, X² is C—Cl, X³ | trifluoromethyl | hydrogen | Y¹ is CH, Y² is N, |
| | is chloro | | | Y³ is CH |
| P.2192 | X¹ is chloro, X² is C—Cl, X³ | trifluoromethyl | methoxy | Y¹ is CH, Y² is N, |
| | is chloro | | | Y³ is CH |
| P.2193 | X¹ is chloro, X² is C—Cl, X³ | trifluoromethyl | methyl | Y¹ is CH, Y² is N, |
| | is chloro | | | Y³ is CH |
| P.2194 | X¹ is chloro, X² is C—Cl, X³ | trifluoromethyl | nitro | Y¹ is CH, Y² is N, |
| | is chloro | | | Y³ is CH |
| P.2195 | X¹ is chloro, X² is C—Cl, X³ | trifluoromethyl | trifluoromethoxy | Y¹ is CH, Y² is N, |
| | is chloro | | | Y³ is CH |
| P.2196 | X¹ is chloro, X² is C—Cl, X³ | trifluoromethyl | trifluoromethyl | Y¹ is CH, Y² is N, |
| | is chloro | | | Y³ is CH |
| P.2197 | X¹ is chloro, X² is C—I, X³ is | chlorodifluoromethyl | bromo | Y¹ is CH, Y² is N, |
| | chloro | | | Y³ is CH |
| P.2198 | X¹ is chloro, X² is C—I, X³ is | chlorodifluoromethyl | chloro | Y¹ is CH, Y² is N, |
| | chloro | | | Y³ is CH |
| P.2199 | X¹ is chloro, X² is C—I, X³ is | chlorodifluoromethyl | cyano | Y¹ is CH, Y² is N, |
| | chloro | | | Y³ is CH |
| P.2200 | X¹ is chloro, X² is C—I, X³ is | chlorodifluoromethyl | cyclopropyl | Y¹ is CH, Y² is N, |
| | chloro | | | Y³ is CH |
| P.2201 | X¹ is chloro, X² is C—I, X³ is | chlorodifluoromethyl | ethyl | Y¹ is CH, Y² is N, |
| | chloro | | | Y³ is CH |
| P.2202 | X¹ is chloro, X² is C—I, X³ is | chlorodifluoromethyl | fluoro | Y¹ is CH, Y² is N, |
| | chloro | | | Y³ is CH |
| P.2203 | X¹ is chloro, X² is C—I, X³ is | chlorodifluoromethyl | hydrogen | Y¹ is CH, Y² is N, |
| | chloro | | | Y³ is CH |
| P.2204 | X¹ is chloro, X² is C—I, X³ is | chlorodifluoromethyl | methoxy | Y¹ is CH, Y² is N, |
| | chloro | | | Y³ is CH |
| P.2205 | X¹ is chloro, X² is C—I, X³ is | chlorodifluoromethyl | methyl | Y¹ is CH, Y² is N, |
| | chloro | | | Y³ is CH |
| P.2206 | X¹ is chloro, X² is C—I, X³ is | chlorodifluoromethyl | nitro | Y¹ is CH, Y² is N, |
| | chloro | | | Y³ is CH |
| P.2207 | X¹ is chloro, X² is C—I, X³ is | chlorodifluoromethyl | trifluoromethoxy | Y¹ is CH, Y² is N, |
| | chloro | | | Y³ is CH |
| P.2208 | X¹ is chloro, X² is C—I, X³ is | chlorodifluoromethyl | trifluoromethyl | Y¹ is CH, Y² is N, |
| | chloro | | | Y³ is CH |
| P.2209 | X¹ is chloro, X² is C—I, X³ is | difluoromethyl | bromo | Y¹ is CH, Y² is N, |
| | chloro | | | Y³ is CH |
| P.2210 | X¹ is chloro, X² is C—I, X³ is | difluoromethyl | chloro | Y¹ is CH, Y² is N, |
| | chloro | | | Y³ is CH |
| P.2211 | X¹ is chloro, X² is C—I, X³ is | difluoromethyl | cyano | Y¹ is CH, Y² is N, |
| | chloro | | | Y³ is CH |
| P.2212 | X¹ is chloro, X² is C—I, X³ is | difluoromethyl | cyclopropyl | Y¹ is CH, Y² is N, |
| | chloro | | | Y³ is CH |
| P.2213 | X¹ is chloro, X² is C—I, X³ is | difluoromethyl | ethyl | Y¹ is CH, Y² is N, |
| | chloro | | | Y³ is CH |
| P.2214 | X¹ is chloro, X² is C—I, X³ is | difluoromethyl | fluoro | Y¹ is CH, Y² is N, |
| | chloro | | | Y³ is CH |
| P.2215 | X¹ is chloro, X² is C—I, X³ is | difluoromethyl | hydrogen | Y¹ is CH, Y² is N, |
| | chloro | | | Y³ is CH |
| P.2216 | X¹ is chloro, X² is C—I, X³ is | difluoromethyl | methoxy | Y¹ is CH, Y² is N, |
| | chloro | | | Y³ is CH |
| P.2217 | X¹ is chloro, X² is C—I, X³ is | difluoromethyl | methyl | Y¹ is CH, Y² is N, |
| | chloro | | | Y³ is CH |
| P.2218 | X¹ is chloro, X² is C—I, X³ is | difluoromethyl | nitro | Y¹ is CH, Y² is N, |
| | chloro | | | Y³ is CH |
| P.2219 | X¹ is chloro, X² is C—I, X³ is | difluoromethyl | trifluoromethoxy | Y¹ is CH, Y² is N, |
| | chloro | | | Y³ is CH |
| P.2220 | X¹ is chloro, X² is C—I, X³ is | difluoromethyl | trifluoromethyl | Y¹ is CH, Y² is N, |
| | chloro | | | Y³ is CH |
| P.2221 | X¹ is chloro, X² is C—I, X³ is | trifluoromethyl | bromo | Y¹ is CH, Y² is N, |
| | chloro | | | Y³ is CH |
| P.2222 | X¹ is chloro, X² is C—I, X³ is | trifluoromethyl | chloro | Y¹ is CH, Y² is N, |
| | chloro | | | Y³ is CH |
| P.2223 | X¹ is chloro, X² is C—I, X³ is | trifluoromethyl | cyano | Y¹ is CH, Y² is N, |
| | chloro | | | Y³ is CH |
| P.2224 | X¹ is chloro, X² is C—I, X³ is | trifluoromethyl | cyclopropyl | Y¹ is CH, Y² is N, |
| | chloro | | | Y³ is CH |
| P.2225 | X¹ is chloro, X² is C—I, X³ is | trifluoromethyl | ethyl | Y¹ is CH, Y² is N, |
| | chloro | | | Y³ is CH |
| P.2226 | X¹ is chloro, X² is C—I, X³ is | trifluoromethyl | fluoro | Y¹ is CH, Y² is N, |
| | chloro | | | Y³ is CH |
| P.2227 | X¹ is chloro, X² is C—I, X³ is | trifluoromethyl | hydrogen | Y¹ is CH, Y² is N, |
| | chloro | | | Y³ is CH |
| P.2228 | X¹ is chloro, X² is C—I, X³ is | trifluoromethyl | methoxy | Y¹ is CH, Y² is N, |
| | chloro | | | Y³ is CH |
| P.2229 | X¹ is chloro, X² is C—I, X³ is | trifluoromethyl | methyl | Y¹ is CH, Y² is N, |
| | chloro | | | Y³ is CH |
| P.2230 | X¹ is chloro, X² is C—I, X³ is | trifluoromethyl | nitro | Y¹ is CH, Y² is N, |
| | chloro | | | Y³ is CH |
| P.2231 | X¹ is chloro, X² is C—I, X³ is | trifluoromethyl | trifluoromethoxy | Y¹ is CH, Y² is N, |
| | chloro | | | Y³ is CH |
| P.2232 | X¹ is chloro, X² is C—I, X³ is | trifluoromethyl | trifluoromethyl | Y¹ is CH, Y² is N, |
| | chloro | | | Y³ is CH |
| P.2233 | X¹ is fluoro, X² is C—F, X³ is | chlorodifluoromethyl | bromo | Y¹ is CH, Y² is N, |
| | fluoro | | | Y³ is CH |
| P.2234 | X¹ is fluoro, X² is C—F, X³ is | chlorodifluoromethyl | chloro | Y¹ is CH, Y² is N, |
| | fluoro | | | Y³ is CH |
| P.2235 | X¹ is fluoro, X² is C—F, X³ is | chlorodifluoromethyl | cyano | Y¹ is CH, Y² is N, |
| | fluoro | | | Y³ is CH |
| P.2236 | X¹ is fluoro, X² is C—F, X³ is | chlorodifluoromethyl | cyclopropyl | Y¹ is CH, Y² is N, |
| | fluoro | | | Y³ is CH |
| P.2237 | X¹ is fluoro, X² is C—F, X³ is | chlorodifluoromethyl | ethyl | Y¹ is CH, Y² is N, |
| | fluoro | | | Y³ is CH |
| P.2238 | X¹ is fluoro, X² is C—F, X³ is | chlorodifluoromethyl | fluoro | Y¹ is CH, Y² is N, |
| | fluoro | | | Y³ is CH |
| P.2239 | X¹ is fluoro, X² is C—F, X³ is | chlorodifluoromethyl | hydrogen | Y¹ is CH, Y² is N, |
| | fluoro | | | Y³ is CH |
| P.2240 | X¹ is fluoro, X² is C—F, X³ is | chlorodifluoromethyl | methoxy | Y¹ is CH, Y² is N, |
| | fluoro | | | Y³ is CH |
| P.2241 | X¹ is fluoro, X² is C—F, X³ is | chlorodifluoromethyl | methyl | Y¹ is CH, Y² is N, |
| | fluoro | | | Y³ is CH |
| P.2242 | X¹ is fluoro, X² is C—F, X³ is | chlorodifluoromethyl | nitro | Y¹ is CH, Y² is N, |
| | fluoro | | | Y³ is CH |
| P.2243 | X¹ is fluoro, X² is C—F, X³ is | chlorodifluoromethyl | trifluoromethoxy | Y¹ is CH, Y² is N, |
| | fluoro | | | Y³ is CH |
| P.2244 | X¹ is fluoro, X² is C—F, X³ is | chlorodifluoromethyl | trifluoromethyl | Y¹ is CH, Y² is N, |
| | fluoro | | | Y³ is CH |
| P.2245 | X¹ is fluoro, X² is C—F, X³ is | difluoromethyl | bromo | Y¹ is CH, Y² is N, |
| | fluoro | | | Y³ is CH |
| P.2246 | X¹ is fluoro, X² is C—F, X³ is | difluoromethyl | chloro | Y¹ is CH, Y² is N, |
| | fluoro | | | Y³ is CH |
| P.2247 | X¹ is fluoro, X² is C—F, X³ is | difluoromethyl | cyano | Y¹ is CH, Y² is N, |
| | fluoro | | | Y³ is CH |
| P.2248 | X¹ is fluoro, X² is C—F, X³ is | difluoromethyl | cyclopropyl | Y¹ is CH, Y² is N, |
| | fluoro | | | Y³ is CH |
| P.2249 | X¹ is fluoro, X² is C—F, X³ is | difluoromethyl | ethyl | Y¹ is CH, Y² is N, |
| | fluoro | | | Y³ is CH |
| P.2250 | X¹ is fluoro, X² is C—F, X³ is | difluoromethyl | fluoro | Y¹ is CH, Y² is N, |
| | fluoro | | | Y³ is CH |
| P.2251 | X¹ is fluoro, X² is C—F, X³ is | difluoromethyl | hydrogen | Y¹ is CH, Y² is N, |
| | fluoro | | | Y³ is CH |
| P.2252 | X¹ is fluoro, X² is C—F, X³ is | difluoromethyl | methoxy | Y¹ is CH, Y² is N, |
| | fluoro | | | Y³ is CH |
| P.2253 | X¹ is fluoro, X² is C—F, X³ is | difluoromethyl | methyl | Y¹ is CH, Y² is N, |
| | fluoro | | | Y³ is CH |
| P.2254 | X¹ is fluoro, X² is C—F, X³ is | difluoromethyl | nitro | Y¹ is CH, Y² is N, |
| | fluoro | | | Y³ is CH |
| P.2255 | X¹ is fluoro, X² is C—F, X³ is | difluoromethyl | trifluoromethoxy | Y¹ is CH, Y² is N, |
| | fluoro | | | Y³ is CH |
| P.2256 | X¹ is fluoro, X² is C—F, X³ is | difluoromethyl | trifluoromethyl | Y¹ is CH, Y² is N, |
| | fluoro | | | Y³ is CH |
| P.2257 | X¹ is fluoro, X² is C—F, X³ is | trifluoromethyl | bromo | Y¹ is CH, Y² is N, |
| | fluoro | | | Y³ is CH |
| P.2258 | X¹ is fluoro, X² is C—F, X³ is | trifluoromethyl | chloro | Y¹ is CH, Y² is N, |
| | fluoro | | | Y³ is CH |
| P.2259 | X¹ is fluoro, X² is C—F, X³ is | trifluoromethyl | cyano | Y¹ is CH, Y² is N, |
| | fluoro | | | Y³ is CH |
| P.2260 | X¹ is fluoro, X² is C—F, X³ is | trifluoromethyl | cyclopropyl | Y¹ is CH, Y² is N, |
| | fluoro | | | Y³ is CH |
| P.2261 | X¹ is fluoro, X² is C—F, X³ is | trifluoromethyl | ethyl | Y¹ is CH, Y² is N, |
| | fluoro | | | Y³ is CH |
| P.2262 | X¹ is fluoro, X² is C—F, X³ is | trifluoromethyl | fluoro | Y¹ is CH, Y² is N, |
| | fluoro | | | Y³ is CH |
| P.2263 | X¹ is fluoro, X² is C—F, X³ is | trifluoromethyl | hydrogen | Y¹ is CH, Y² is N, |
| | fluoro | | | Y³ is CH |
| P.2264 | X¹ is fluoro, X² is C—F, X³ is | trifluoromethyl | methoxy | Y¹ is CH, Y² is N, |
| | fluoro | | | Y³ is CH |
| P.2265 | X¹ is fluoro, X² is C—F, X³ is | trifluoromethyl | methyl | Y¹ is CH, Y² is N, |
| | fluoro | | | Y³ is CH |
| P.2266 | X¹ is fluoro, X² is C—F, X³ is | trifluoromethyl | nitro | Y¹ is CH, Y² is N, |
| | fluoro | | | Y³ is CH |
| P.2267 | X¹ is fluoro, X² is C—F, X³ is | trifluoromethyl | trifluoromethoxy | Y¹ is CH, Y² is N, |
| | fluoro | | | Y³ is CH |
| P.2268 | X¹ is fluoro, X² is C—F, X³ is | trifluoromethyl | trifluoromethyl | Y¹ is CH, Y² is N, |
| | fluoro | | | Y³ is CH |
| P.2269 | X¹ is chloro, X² is CH, X³ is | chlorodifluoromethyl | bromo | Y¹ is CH, Y² is N, |
| | bromo | | | Y³ is CH |
| P.2270 | X¹ is chloro, X² is CH, X³ is | chlorodifluoromethyl | chloro | Y¹ is CH, Y² is N, |
| | bromo | | | Y³ is CH |
| P.2271 | X¹ is chloro, X² is CH, X³ is | chlorodifluoromethyl | cyano | Y¹ is CH, Y² is N, |
| | bromo | | | Y³ is CH |
| P.2272 | X¹ is chloro, X² is CH, X³ is | chlorodifluoromethyl | cyclopropyl | Y¹ is CH, Y² is N, |
| | bromo | | | Y³ is CH |
| P.2273 | X¹ is chloro, X² is CH, X³ is | chlorodifluoromethyl | ethyl | Y¹ is CH, Y² is N, |
| | bromo | | | Y³ is CH |
| P.2274 | X¹ is chloro, X² is CH, X³ is | chlorodifluoromethyl | fluoro | Y¹ is CH, Y² is N, |
| | bromo | | | Y³ is CH |
| P.2275 | X¹ is chloro, X² is CH, X³ is | chlorodifluoromethyl | hydrogen | Y¹ is CH, Y² is N, |
| | bromo | | | Y³ is CH |
| P.2276 | X¹ is chloro, X² is CH, X³ is | chlorodifluoromethyl | methoxy | Y¹ is CH, Y² is N, |
| | bromo | | | Y³ is CH |
| P.2277 | X¹ is chloro, X² is CH, X³ is | chlorodifluoromethyl | methyl | Y¹ is CH, Y² is N, |
| | bromo | | | Y³ is CH |
| P.2278 | X¹ is chloro, X² is CH, X³ is | chlorodifluoromethyl | nitro | Y¹ is CH, Y² is N, |
| | bromo | | | Y³ is CH |
| P.2279 | X¹ is chloro, X² is CH, X³ is | chlorodifluoromethyl | trifluoromethoxy | Y¹ is CH, Y² is N, |
| | bromo | | | Y³ is CH |
| P.2280 | X¹ is chloro, X² is CH, X³ is | chlorodifluoromethyl | trifluoromethyl | Y¹ is CH, Y² is N, |
| | bromo | | | Y³ is CH |
| P.2281 | X¹ is chloro, X² is CH, X³ is | difluoromethyl | bromo | Y¹ is CH, Y² is N, |
| | bromo | | | Y³ is CH |
| P.2282 | X¹ is chloro, X² is CH, X³ is | difluoromethyl | chloro | Y¹ is CH, Y² is N, |
| | bromo | | | Y³ is CH |
| P.2283 | X¹ is chloro, X² is CH, X³ is | difluoromethyl | cyano | Y¹ is CH, Y² is N, |
| | bromo | | | Y³ is CH |
| P.2284 | X¹ is chloro, X² is CH, X³ is | difluoromethyl | cyclopropyl | Y¹ is CH, Y² is N, |
| | bromo | | | Y³ is CH |
| P.2285 | X¹ is chloro, X² is CH, X³ is | difluoromethyl | ethyl | Y¹ is CH, Y² is N, |
| | bromo | | | Y³ is CH |
| P.2286 | X¹ is chloro, X² is CH, X³ is | difluoromethyl | fluoro | Y¹ is CH, Y² is N, |
| | bromo | | | Y³ is CH |
| P.2287 | X¹ is chloro, X² is CH, X³ is | difluoromethyl | hydrogen | Y¹ is CH, Y² is N, |
| | bromo | | | Y³ is CH |
| P.2288 | X¹ is chloro, X² is CH, X³ is | difluoromethyl | methoxy | Y¹ is CH, Y² is N, |
| | bromo | | | Y³ is CH |
| P.2289 | X¹ is chloro, X² is CH, X³ is | difluoromethyl | methyl | Y¹ is CH, Y² is N, |
| | bromo | | | Y³ is CH |
| P.2290 | X¹ is chloro, X² is CH, X³ is | difluoromethyl | nitro | Y¹ is CH, Y² is N, |
| | bromo | | | Y³ is CH |
| P.2291 | X¹ is chloro, X² is CH, X³ is | difluoromethyl | trifluoromethoxy | Y¹ is CH, Y² is N, |
| | bromo | | | Y³ is CH |
| P.2292 | X¹ is chloro, X² is CH, X³ is | difluoromethyl | trifluoromethyl | Y¹ is CH, Y² is N, |
| | bromo | | | Y³ is CH |
| P.2293 | X¹ is chloro, X² is CH, X³ is | trifluoromethyl | bromo | Y¹ is CH, Y² is N, |
| | bromo | | | Y³ is CH |
| P.2294 | X¹ is chloro, X² is CH, X³ is | trifluoromethyl | chloro | Y¹ is CH, Y² is N, |
| | bromo | | | Y³ is CH |
| P.2295 | X¹ is chloro, X² is CH, X³ is | trifluoromethyl | cyano | Y¹ is CH, Y² is N, |
| | bromo | | | Y³ is CH |
| P.2296 | X¹ is chloro, X² is CH, X³ is | trifluoromethyl | cyclopropyl | Y¹ is CH, Y² is N, |
| | bromo | | | Y³ is CH |
| P.2297 | X¹ is chloro, X² is CH, X³ is | trifluoromethyl | ethyl | Y¹ is CH, Y² is N, |
| | bromo | | | Y³ is CH |
| P.2298 | X¹ is chloro, X² is CH, X³ is | trifluoromethyl | fluoro | Y¹ is CH, Y² is N, |
| | bromo | | | Y³ is CH |
| P.2299 | X¹ is chloro, X² is CH, X³ is | trifluoromethyl | hydrogen | Y¹ is CH, Y² is N, |
| | bromo | | | Y³ is CH |
| P.2300 | X¹ is chloro, X² is CH, X³ is | trifluoromethyl | methoxy | Y¹ is CH, Y² is N, |
| | bromo | | | Y³ is CH |
| P.2301 | X¹ is chloro, X² is CH, X³ is | trifluoromethyl | methyl | Y¹ is CH, Y² is N, |
| | bromo | | | Y³ is CH |
| P.2302 | X¹ is chloro, X² is CH, X³ is | trifluoromethyl | nitro | Y¹ is CH, Y² is N, |
| | bromo | | | Y³ is CH |
| P.2303 | X¹ is chloro, X² is CH, X³ is | trifluoromethyl | trifluoromethoxy | Y¹ is CH, Y² is N, |
| | bromo | | | Y³ is CH |
| P.2304 | X¹ is chloro, X² is CH, X³ is | trifluoromethyl | trifluoromethyl | Y¹ is CH, Y² is N, |
| | bromo | | | Y³ is CH |
| P.2305 | X¹ is chloro, X² is CH, X³ is | chlorodifluoromethyl | bromo | Y¹ is CH, Y² is N, |
| | bromo | | | Y³ is CH |
| P.2306 | X¹ is chloro, X² is CH, X³ is | chlorodifluoromethyl | chloro | Y¹ is CH, Y² is N, |
| | bromo | | | Y³ is CH |
| P.2307 | X¹ is chloro, X² is CH, X³ is | chlorodifluoromethyl | cyano | Y¹ is CH, Y² is N, |
| | bromo | | | Y³ is CH |
| P.2308 | X¹ is chloro, X² is CH, X³ is | chlorodifluoromethyl | cyclopropyl | Y¹ is CH, Y² is N, |
| | bromo | | | Y³ is CH |
| P.2309 | X¹ is chloro, X² is CH, X³ is | chlorodifluoromethyl | ethyl | Y¹ is CH, Y² is N, |
| | bromo | | | Y³ is CH |
| P.2310 | X¹ is chloro, X² is CH, X³ is | chlorodifluoromethyl | fluoro | Y¹ is CH, Y² is N, |
| | bromo | | | Y³ is CH |
| P.2311 | X¹ is chloro, X² is CH, X³ is | chlorodifluoromethyl | hydrogen | Y¹ is CH, Y² is N, |
| | bromo | | | Y³ is CH |
| P.2312 | X¹ is chloro, X² is CH, X³ is | chlorodifluoromethyl | methoxy | Y¹ is CH, Y² is N, |
| | bromo | | | Y³ is CH |
| P.2313 | X¹ is chloro, X² is CH, X³ is | chlorodifluoromethyl | methyl | Y¹ is CH, Y² is N, |
| | bromo | | | Y³ is CH |
| P.2314 | X¹ is chloro, X² is CH, X³ is | chlorodifluoromethyl | nitro | Y¹ is CH, Y² is N, |
| | bromo | | | Y³ is CH |
| P.2315 | X¹ is chloro, X² is CH, X³ is | chlorodifluoromethyl | trifluoromethoxy | Y¹ is CH, Y² is N, |
| | bromo | | | Y³ is CH |
| P.2316 | X¹ is chloro, X² is CH, X³ is | chlorodifluoromethyl | trifluoromethyl | Y¹ is CH, Y² is N, |
| | bromo | | | Y³ is CH |
| P.2317 | X¹ is chloro, X² is CH, X³ is | difluoromethyl | bromo | Y¹ is CH, Y² is N, |
| | bromo | | | Y³ is CH |
| P.2318 | X¹ is chloro, X² is CH, X³ is | difluoromethyl | chloro | Y¹ is CH, Y² is N, |
| | bromo | | | Y³ is CH |
| P.2319 | X¹ is chloro, X² is CH, X³ is | difluoromethyl | cyano | Y¹ is CH, Y² is N, |
| | bromo | | | Y³ is CH |
| P.2320 | X¹ is chloro, X² is CH, X³ is | difluoromethyl | cyclopropyl | Y¹ is CH, Y² is N, |
| | bromo | | | Y³ is CH |
| P.2321 | X¹ is chloro, X² is CH, X³ is | difluoromethyl | ethyl | Y¹ is CH, Y² is N, |
| | bromo | | | Y³ is CH |
| P.2322 | X¹ is chloro, X² is CH, X³ is | difluoromethyl | fluoro | Y¹ is CH, Y² is N, |
| | bromo | | | Y³ is CH |
| P.2323 | X¹ is chloro, X² is CH, X³ is | difluoromethyl | hydrogen | Y¹ is CH, Y² is N, |
| | bromo | | | Y³ is CH |
| P.2324 | X¹ is chloro, X² is CH, X³ is | difluoromethyl | methoxy | Y¹ is CH, Y² is N, |
| | bromo | | | Y³ is CH |
| P.2325 | X¹ is chloro, X² is CH, X³ is | difluoromethyl | methyl | Y¹ is CH, Y² is N, |
| | bromo | | | Y³ is CH |
| P.2326 | X¹ is chloro, X² is CH, X³ is | difluoromethyl | nitro | Y¹ is CH, Y² is N, |
| | bromo | | | Y³ is CH |
| P.2327 | X¹ is chloro, X² is CH, X³ is | difluoromethyl | trifluoromethoxy | Y¹ is CH, Y² is N, |
| | bromo | | | Y³ is CH |
| P.2328 | X¹ is chloro, X² is CH, X³ is | difluoromethyl | trifluoromethyl | Y¹ is CH, Y² is N, |
| | bromo | | | Y³ is CH |
| P.2329 | X¹ is chloro, X² is CH, X³ is | trifluoromethyl | bromo | Y¹ is CH, Y² is N, |
| | bromo | | | Y³ is CH |
| P.2330 | X¹ is chloro, X² is CH, X³ is | trifluoromethyl | chloro | Y¹ is CH, Y² is N, |
| | bromo | | | Y³ is CH |
| P.2331 | X¹ is chloro, X² is CH, X³ is | trifluoromethyl | cyano | Y¹ is CH, Y² is N, |
| | bromo | | | Y³ is CH |
| P.2332 | X¹ is chloro, X² is CH, X³ is | trifluoromethyl | cyclopropyl | Y¹ is CH, Y² is N, |
| | bromo | | | Y³ is CH |
| P.2333 | X¹ is chloro, X² is CH, X³ is | trifluoromethyl | ethyl | Y¹ is CH, Y² is N, |
| | bromo | | | Y³ is CH |
| P.2334 | X¹ is chloro, X² is CH, X³ is | trifluoromethyl | fluoro | Y¹ is CH, Y² is N, |
| | bromo | | | Y³ is CH |
| P.2335 | X¹ is chloro, X² is CH, X³ is | trifluoromethyl | hydrogen | Y¹ is CH, Y² is N, |
| | bromo | | | Y³ is CH |
| P.2336 | X¹ is chloro, X² is CH, X³ is | trifluoromethyl | methoxy | Y¹ is CH, Y² is N, |
| | bromo | | | Y³ is CH |
| P.2337 | X¹ is chloro, X² is CH, X³ is | trifluoromethyl | methyl | Y¹ is CH, Y² is N, |
| | bromo | | | Y³ is CH |
| P.2338 | X¹ is chloro, X² is CH, X³ is | trifluoromethyl | nitro | Y¹ is CH, Y² is N, |
| | bromo | | | Y³ is CH |
| P.2339 | X¹ is chloro, X² is CH, X³ is | trifluoromethyl | trifluoromethoxy | Y¹ is CH, Y² is N, |
| | bromo | | | Y³ is CH |
| P.2340 | X¹ is chloro, X² is CH, X³ is | trifluoromethyl | trifluoromethyl | Y¹ is CH, Y² is N, |
| | bromo | | | Y³ is CH |
| P.2341 | X¹ is chloro, X² is CH, X³ is | chlorodifluoromethyl | bromo | Y¹ is CH, Y² is N, |
| | bromo | | | Y³ is CH |
| P.2342 | X¹ is chloro, X² is CH, X³ is | chlorodifluoromethyl | chloro | Y¹ is CH, Y² is N, |
| | bromo | | | Y³ is CH |
| P.2343 | X¹ is chloro, X² is CH, X³ is | chlorodifluoromethyl | cyano | Y¹ is CH, Y² is N, |
| | bromo | | | Y³ is CH |
| P.2344 | X¹ is chloro, X² is CH, X³ is | chlorodifluoromethyl | cyclopropyl | Y¹ is CH, Y² is N, |
| | bromo | | | Y³ is CH |
| P.2345 | X¹ is chloro, X² is CH, X³ is | chlorodifluoromethyl | ethyl | Y¹ is CH, Y² is N, |
| | bromo | | | Y³ is CH |
| P.2346 | X¹ is chloro, X² is CH, X³ is | chlorodifluoromethyl | fluoro | Y¹ is CH, Y² is N, |
| | bromo | | | Y³ is CH |
| P.2347 | X¹ is chloro, X² is CH, X³ is | chlorodifluoromethyl | hydrogen | Y¹ is CH, Y² is N, |
| | bromo | | | Y³ is CH |
| P.2348 | X¹ is chloro, X² is CH, X³ is | chlorodifluoromethyl | methoxy | Y¹ is CH, Y² is N, |
| | bromo | | | Y³ is CH |
| P.2349 | X¹ is chloro, X² is CH, X³ is | chlorodifluoromethyl | methyl | Y¹ is CH, Y² is N, |
| | bromo | | | Y³ is CH |
| P.2350 | X¹ is chloro, X² is CH, X³ is | chlorodifluoromethyl | nitro | Y¹ is CH, Y² is N, |
| | bromo | | | Y³ is CH |
| P.2351 | X¹ is chloro, X² is CH, X³ is | chlorodifluoromethyl | trifluoromethoxy | Y¹ is CH, Y² is N, |
| | bromo | | | Y³ is CH |
| P.2352 | X¹ is chloro, X² is CH, X³ is | chlorodifluoromethyl | trifluoromethyl | Y¹ is CH, Y² is N, |
| | bromo | | | Y³ is CH |
| P.2353 | X¹ is chloro, X² is CH, X³ is | difluoromethyl | bromo | Y¹ is CH, Y² is N, |
| | bromo | | | Y³ is CH |
| P.2354 | X¹ is chloro, X² is CH, X³ is | difluoromethyl | chloro | Y¹ is CH, Y² is N, |
| | bromo | | | Y³ is CH |
| P.2355 | X¹ is chloro, X² is CH, X³ is | difluoromethyl | cyano | Y¹ is CH, Y² is N, |
| | bromo | | | Y³ is CH |
| P.2356 | X¹ is chloro, X² is CH, X³ is | difluoromethyl | cyclopropyl | Y¹ is CH, Y² is N, |
| | bromo | | | Y³ is CH |
| P.2357 | X¹ is chloro, X² is CH, X³ is | difluoromethyl | ethyl | Y¹ is CH, Y² is N, |
| | bromo | | | Y³ is CH |
| P.2358 | X¹ is chloro, X² is CH, X³ is | difluoromethyl | fluoro | Y¹ is CH, Y² is N, |
| | bromo | | | Y³ is CH |
| P.2359 | X¹ is chloro, X² is CH, X³ is | difluoromethyl | hydrogen | Y¹ is CH, Y² is N, |
| | bromo | | | Y³ is CH |
| P.2360 | X¹ is chloro, X² is CH, X³ is | difluoromethyl | methoxy | Y¹ is CH, Y² is N, |
| | bromo | | | Y³ is CH |
| P.2361 | X¹ is chloro, X² is CH, X³ is | difluoromethyl | methyl | Y¹ is CH, Y² is N, |
| | bromo | | | Y³ is CH |
| P.2362 | X¹ is chloro, X² is CH, X³ is | difluoromethyl | nitro | Y¹ is CH, Y² is N, |
| | bromo | | | Y³ is CH |
| P.2363 | X¹ is chloro, X² is CH, X³ is | difluoromethyl | trifluoromethoxy | Y¹ is CH, Y² is N, |
| | bromo | | | Y³ is CH |
| P.2364 | X¹ is chloro, X² is CH, X³ is | difluoromethyl | trifluoromethyl | Y¹ is CH, Y² is N, |
| | bromo | | | Y³ is CH |
| P.2365 | X¹ is chloro, X² is CH, X³ is | trifluoromethyl | bromo | Y¹ is CH, Y² is N, |
| | bromo | | | Y³ is CH |
| P.2366 | X¹ is chloro, X² is CH, X³ is | trifluoromethyl | chloro | Y¹ is CH, Y² is N, |
| | bromo | | | Y³ is CH |
| P.2367 | X¹ is chloro, X² is CH, X³ is | trifluoromethyl | cyano | Y¹ is CH, Y² is N, |
| | bromo | | | Y³ is CH |
| P.2368 | X¹ is chloro, X² is CH, X³ is | trifluoromethyl | cyclopropyl | Y¹ is CH, Y² is N, |
| | bromo | | | Y³ is CH |
| P.2369 | X¹ is chloro, X² is CH, X³ is | trifluoromethyl | ethyl | Y¹ is CH, Y² is N, |
| | bromo | | | Y³ is CH |
| P.2370 | X¹ is chloro, X² is CH, X³ is | trifluoromethyl | fluoro | Y¹ is CH, Y² is N, |
| | bromo | | | Y³ is CH |
| P.2371 | X¹ is chloro, X² is CH, X³ is | trifluoromethyl | hydrogen | Y¹ is CH, Y² is N, |
| | bromo | | | Y³ is CH |
| P.2372 | X¹ is chloro, X² is CH, X³ is | trifluoromethyl | methoxy | Y¹ is CH, Y² is N, |
| | bromo | | | Y³ is CH |
| P.2373 | X¹ is chloro, X² is CH, X³ is | trifluoromethyl | methyl | Y¹ is CH, Y² is N, |
| | bromo | | | Y³ is CH |
| P.2374 | X¹ is chloro, X² is CH, X³ is | trifluoromethyl | nitro | Y¹ is CH, Y² is N, |
| | bromo | | | Y³ is CH |
| P.2375 | X¹ is chloro, X² is CH, X³ is | trifluoromethyl | trifluoromethoxy | Y¹ is CH, Y² is N, |
| | bromo | | | Y³ is CH |
| P.2376 | X¹ is chloro, X² is CH, X³ is | trifluoromethyl | trifluoromethyl | Y¹ is CH, Y² is N, |
| | bromo | | | Y³ is CH |
| P.2377 | X¹ is chloro, X² is C—Cl, X³ | chlorodifluoromethyl | bromo | Y¹ is CH, Y² is N, |
| | is trifluoromethyl | | | Y³ is CH |
| P.2378 | X¹ is chloro, X² is C—Cl, X³ | chlorodifluoromethyl | chloro | Y¹ is CH, Y² is N, |
| | is trifluoromethyl | | | Y³ is CH |
| P.2379 | X¹ is chloro, X² is C—Cl, X³ | chlorodifluoromethyl | cyano | Y¹ is CH, Y² is N, |
| | is trifluoromethyl | | | Y³ is CH |
| P.2380 | X¹ is chloro, X² is C—Cl, X³ | chlorodifluoromethyl | cyclopropyl | Y¹ is CH, Y² is N, |
| | is trifluoromethyl | | | Y³ is CH |
| P.2381 | X¹ is chloro, X² is C—Cl, X³ | chlorodifluoromethyl | ethyl | Y¹ is CH, Y² is N, |
| | is trifluoromethyl | | | Y³ is CH |
| P.2382 | X¹ is chloro, X² is C—Cl, X³ | chlorodifluoromethyl | fluoro | Y¹ is CH, Y² is N, |
| | is trifluoromethyl | | | Y³ is CH |
| P.2383 | X¹ is chloro, X² is C—Cl, X³ | chlorodifluoromethyl | hydrogen | Y¹ is CH, Y² is N, |
| | is trifluoromethyl | | | Y³ is CH |
| P.2384 | X¹ is chloro, X² is C—Cl, X³ | chlorodifluoromethyl | methoxy | Y¹ is CH, Y² is N, |
| | is trifluoromethyl | | | Y³ is CH |
| P.2385 | X¹ is chloro, X² is C—Cl, X³ | chlorodifluoromethyl | methyl | Y¹ is CH, Y² is N, |
| | is trifluoromethyl | | | Y³ is CH |
| P.2386 | X¹ is chloro, X² is C—Cl, X³ | chlorodifluoromethyl | nitro | Y¹ is CH, Y² is N, |
| | is trifluoromethyl | | | Y³ is CH |
| P.2387 | X¹ is chloro, X² is C—Cl, X³ | chlorodifluoromethyl | trifluoromethoxy | Y¹ is CH, Y² is N, |
| | is trifluoromethyl | | | Y³ is CH |
| P.2388 | X¹ is chloro, X² is C—Cl, X³ | chlorodifluoromethyl | trifluoromethyl | Y¹ is CH, Y² is N, |
| | is trifluoromethyl | | | Y³ is CH |
| P.2389 | X¹ is chloro, X² is C—Cl, X³ | difluoromethyl | bromo | Y¹ is CH, Y² is N, |
| | is trifluoromethyl | | | Y³ is CH |
| P.2390 | X¹ is chloro, X² is C—Cl, X³ | difluoromethyl | chloro | Y¹ is CH, Y² is N, |
| | is trifluoromethyl | | | Y³ is CH |
| P.2391 | X¹ is chloro, X² is C—Cl, X³ | difluoromethyl | cyano | Y¹ is CH, Y² is N, |
| | is trifluoromethyl | | | Y³ is CH |
| P.2392 | X¹ is chloro, X² is C—Cl, X³ | difluoromethyl | cyclopropyl | Y¹ is CH, Y² is N, |
| | is trifluoromethyl | | | Y³ is CH |
| P.2393 | X¹ is chloro, X² is C—Cl, X³ | difluoromethyl | ethyl | Y¹ is CH, Y² is N, |
| | is trifluoromethyl | | | Y³ is CH |
| P.2394 | X¹ is chloro, X² is C—Cl, X³ | difluoromethyl | fluoro | Y¹ is CH, Y² is N, |
| | is trifluoromethyl | | | Y³ is CH |
| P.2395 | X¹ is chloro, X² is C—Cl, X³ | difluoromethyl | hydrogen | Y¹ is CH, Y² is N, |
| | is trifluoromethyl | | | Y³ is CH |
| P.2396 | X¹ is chloro, X² is C—Cl, X³ | difluoromethyl | methoxy | Y¹ is CH, Y² is N, |
| | is trifluoromethyl | | | Y³ is CH |
| P.2397 | X¹ is chloro, X² is C—Cl, X³ | difluoromethyl | methyl | Y¹ is CH, Y² is N, |
| | is trifluoromethyl | | | Y³ is CH |
| P.2398 | X¹ is chloro, X² is C—Cl, X³ | difluoromethyl | nitro | Y¹ is CH, Y² is N, |
| | is trifluoromethyl | | | Y³ is CH |
| P.2399 | X¹ is chloro, X² is C—Cl, X³ | difluoromethyl | trifluoromethoxy | Y¹ is CH, Y² is N, |
| | is trifluoromethyl | | | Y³ is CH |
| P.2400 | X¹ is chloro, X² is C—Cl, X³ | difluoromethyl | trifluoromethyl | Y¹ is CH, Y² is N, |
| | is trifluoromethyl | | | Y³ is CH |
| P.2401 | X¹ is chloro, X² is C—Cl, X³ | trifluoromethyl | bromo | Y¹ is CH, Y² is N, |
| | is trifluoromethyl | | | Y³ is CH |
| P.2402 | X¹ is chloro, X² is C—Cl, X³ | trifluoromethyl | chloro | Y¹ is CH, Y² is N, |
| | is trifluoromethyl | | | Y³ is CH |
| P.2403 | X¹ is chloro, X² is C—Cl, X³ | trifluoromethyl | cyano | Y¹ is CH, Y² is N, |
| | is trifluoromethyl | | | Y³ is CH |
| P.2404 | X¹ is chloro, X² is C—Cl, X³ | trifluoromethyl | cyclopropyl | Y¹ is CH, Y² is N, |
| | is trifluoromethyl | | | Y³ is CH |
| P.2405 | X¹ is chloro, X² is C—Cl, X³ | trifluoromethyl | ethyl | Y¹ is CH, Y² is N, |
| | is trifluoromethyl | | | Y³ is CH |
| P.2406 | X¹ is chloro, X² is C—Cl, X³ | trifluoromethyl | fluoro | Y¹ is CH, Y² is N, |
| | is trifluoromethyl | | | Y³ is CH |
| P.2407 | X¹ is chloro, X² is C—Cl, X³ | trifluoromethyl | hydrogen | Y¹ is CH, Y² is N, |
| | is trifluoromethyl | | | Y³ is CH |
| P.2408 | X¹ is chloro, X² is C—Cl, X³ | trifluoromethyl | methoxy | Y¹ is CH, Y² is N, |
| | is trifluoromethyl | | | Y³ is CH |
| P.2409 | X¹ is chloro, X² is C—Cl, X³ | trifluoromethyl | methyl | Y¹ is CH, Y² is N, |
| | is trifluoromethyl | | | Y³ is CH |
| P.2410 | X¹ is chloro, X² is C—Cl, X³ | trifluoromethyl | nitro | Y¹ is CH, Y² is N, |
| | is trifluoromethyl | | | Y³ is CH |
| P.2411 | X¹ is chloro, X² is C—Cl, X³ | trifluoromethyl | trifluoromethoxy | Y¹ is CH, Y² is N, |
| | is trifluoromethyl | | | Y³ is CH |
| P.2412 | X¹ is chloro, X² is C—Cl, X³ | trifluoromethyl | trifluoromethyl | Y¹ is CH, Y² is N, |
| | is trifluoromethyl | | | Y³ is CH |
| P.2413 | X¹ is trifluoromethyl, X² is | chlorodifluoromethyl | bromo | Y¹ is CH, Y² is N, |
| | CH, X³ is trifluoromethyl | | | Y³ is CH |
| P.2414 | X¹ is trifluoromethyl, X² is | chlorodifluoromethyl | chloro | Y¹ is CH, Y² is N, |
| | CH, X³ is trifluoromethyl | | | Y³ is CH |
| P.2415 | X¹ is trifluoromethyl, X² is | chlorodifluoromethyl | cyano | Y¹ is CH, Y² is N, |
| | CH, X³ is trifluoromethyl | | | Y³ is CH |
| P.2416 | X¹ is trifluoromethyl, X² is | chlorodifluoromethyl | cyclopropyl | Y¹ is CH, Y² is N, |
| | CH, X³ is trifluoromethyl | | | Y³ is CH |
| P.2417 | X¹ is trifluoromethyl, X² is | chlorodifluoromethyl | ethyl | Y¹ is CH, Y² is N, |
| | CH, X³ is trifluoromethyl | | | Y³ is CH |
| P.2418 | X¹ is trifluoromethyl, X² is | chlorodifluoromethyl | fluoro | Y¹ is CH, Y² is N, |
| | CH, X³ is trifluoromethyl | | | Y³ is CH |
| P.2419 | X¹ is trifluoromethyl, X² is | chlorodifluoromethyl | hydrogen | Y¹ is CH, Y² is N, |
| | CH, X³ is trifluoromethyl | | | Y³ is CH |
| P.2420 | X¹ is trifluoromethyl, X² is | chlorodifluoromethyl | methoxy | Y¹ is CH, Y² is N, |
| | CH, X³ is trifluoromethyl | | | Y³ is CH |
| P.2421 | X¹ is trifluoromethyl, X² is | chlorodifluoromethyl | methyl | Y¹ is CH, Y² is N, |
| | CH, X³ is trifluoromethyl | | | Y³ is CH |
| P.2422 | X¹ is trifluoromethyl, X² is | chlorodifluoromethyl | nitro | Y¹ is CH, Y² is N, |
| | CH, X³ is trifluoromethyl | | | Y³ is CH |
| P.2423 | X¹ is trifluoromethyl, X² is | chlorodifluoromethyl | trifluoromethoxy | Y¹ is CH, Y² is N, |
| | CH, X³ is trifluoromethyl | | | Y³ is CH |
| P.2424 | X¹ is trifluoromethyl, X² is | chlorodifluoromethyl | trifluoromethyl | Y¹ is CH, Y² is N, |
| | CH, X³ is trifluoromethyl | | | Y³ is CH |
| P.2425 | X¹ is trifluoromethyl, X² is | difluoromethyl | bromo | Y¹ is CH, Y² is N, |
| | CH, X³ is trifluoromethyl | | | Y³ is CH |
| P.2426 | X¹ is trifluoromethyl, X² is | difluoromethyl | chloro | Y¹ is CH, Y² is N, |
| | CH, X³ is trifluoromethyl | | | Y³ is CH |
| P.2427 | X¹ is trifluoromethyl, X² is | difluoromethyl | cyano | Y¹ is CH, Y² is N, |
| | CH, X³ is trifluoromethyl | | | Y³ is CH |
| P.2428 | X¹ is trifluoromethyl, X² is | difluoromethyl | cyclopropyl | Y¹ is CH, Y² is N, |
| | CH, X³ is trifluoromethyl | | | Y³ is CH |
| P.2429 | X¹ is trifluoromethyl, X² is | difluoromethyl | ethyl | Y¹ is CH, Y² is N, |
| | CH, X³ is trifluoromethyl | | | Y³ is CH |
| P.2430 | X¹ is trifluoromethyl, X² is | difluoromethyl | fluoro | Y¹ is CH, Y² is N, |
| | CH, X³ is trifluoromethyl | | | Y³ is CH |
| P.2431 | X¹ is trifluoromethyl, X² is | difluoromethyl | hydrogen | Y¹ is CH, Y² is N, |
| | CH, X³ is trifluoromethyl | | | Y³ is CH |
| P.2432 | X¹ is trifluoromethyl, X² is | difluoromethyl | methoxy | Y¹ is CH, Y² is N, |
| | CH, X³ is trifluoromethyl | | | Y³ is CH |
| P.2433 | X¹ is trifluoromethyl, X² is | difluoromethyl | methyl | Y¹ is CH, Y² is N, |
| | CH, X³ is trifluoromethyl | | | Y³ is CH |
| P.2434 | X¹ is trifluoromethyl, X² is | difluoromethyl | nitro | Y¹ is CH, Y² is N, |
| | CH, X³ is trifluoromethyl | | | Y³ is CH |
| P.2435 | X¹ is trifluoromethyl, X² is | difluoromethyl | trifluoromethoxy | Y¹ is CH, Y² is N, |
| | CH, X³ is trifluoromethyl | | | Y³ is CH |
| P.2436 | X¹ is trifluoromethyl, X² is | difluoromethyl | trifluoromethyl | Y¹ is CH, Y² is N, |
| | CH, X³ is trifluoromethyl | | | Y³ is CH |
| P.2437 | X¹ is trifluoromethyl, X² is | trifluoromethyl | bromo | Y¹ is CH, Y² is N, |
| | CH, X³ is trifluoromethyl | | | Y³ is CH |
| P.2438 | X¹ is trifluoromethyl, X² is | trifluoromethyl | chloro | Y¹ is CH, Y² is N, |
| | CH, X³ is trifluoromethyl | | | Y³ is CH |
| P.2439 | X¹ is trifluoromethyl, X² is | trifluoromethyl | cyano | Y¹ is CH, Y² is N, |
| | CH, X³ is trifluoromethyl | | | Y³ is CH |
| P.2440 | X¹ is trifluoromethyl, X² is | trifluoromethyl | cyclopropyl | Y¹ is CH, Y² is N, |
| | CH, X³ is trifluoromethyl | | | Y³ is CH |
| P.2441 | X¹ is trifluoromethyl, X² is | trifluoromethyl | ethyl | Y¹ is CH, Y² is N, |
| | CH, X³ is trifluoromethyl | | | Y³ is CH |
| P.2442 | X¹ is trifluoromethyl, X² is | trifluoromethyl | fluoro | Y¹ is CH, Y² is N, |
| | CH, X³ is trifluoromethyl | | | Y³ is CH |
| P.2443 | X¹ is trifluoromethyl, X² is | trifluoromethyl | hydrogen | Y¹ is CH, Y² is N, |
| | CH, X³ is trifluoromethyl | | | Y³ is CH |
| P.2444 | X¹ is trifluoromethyl, X² is | trifluoromethyl | methoxy | Y¹ is CH, Y² is N, |
| | CH, X³ is trifluoromethyl | | | Y³ is CH |
| P.2445 | X¹ is trifluoromethyl, X² is | trifluoromethyl | methyl | Y¹ is CH, Y² is N, |
| | CH, X³ is trifluoromethyl | | | Y³ is CH |
| P.2446 | X¹ is trifluoromethyl, X² is | trifluoromethyl | nitro | Y¹ is CH, Y² is N, |
| | CH, X³ is trifluoromethyl | | | Y³ is CH |
| P.2447 | X¹ is trifluoromethyl, X² is | trifluoromethyl | trifluoromethoxy | Y¹ is CH, Y² is N, |
| | CH, X³ is trifluoromethyl | | | Y³ is CH |
| P.2448 | X¹ is trifluoromethyl, X² is | trifluoromethyl | trifluoromethyl | Y¹ is CH, Y² is N, |
| | CH, X³ is trifluoromethyl | | | Y³ is CH |
| P.2449 | X¹ is trifluoromethyl, X² is | chlorodifluoromethyl | bromo | Y¹ is CH, Y² is N, |
| | CH, X³ is trifluoromethyl | | | Y³ is CH |
| P.2450 | X¹ is trifluoromethyl, X² is | chlorodifluoromethyl | chloro | Y¹ is CH, Y² is N, |
| | CH, X³ is trifluoromethyl | | | Y³ is CH |
| P.2451 | X¹ is trifluoromethyl, X² is | chlorodifluoromethyl | cyano | Y¹ is CH, Y² is N, |
| | CH, X³ is trifluoromethyl | | | Y³ is CH |
| P.2452 | X¹ is trifluoromethyl, X² is | chlorodifluoromethyl | cyclopropyl | Y¹ is CH, Y² is N, |
| | CH, X³ is trifluoromethyl | | | Y³ is CH |
| P.2453 | X¹ is trifluoromethyl, X² is | chlorodifluoromethyl | ethyl | Y¹ is CH, Y² is N, |
| | CH, X³ is trifluoromethyl | | | Y³ is CH |
| P.2454 | X¹ is trifluoromethyl, X² is | chlorodifluoromethyl | fluoro | Y¹ is CH, Y² is N, |
| | CH, X³ is trifluoromethyl | | | Y³ is CH |
| P.2455 | X¹ is trifluoromethyl, X² is | chlorodifluoromethyl | hydrogen | Y¹ is CH, Y² is N, |
| | CH, X³ is trifluoromethyl | | | Y³ is CH |
| P.2456 | X¹ is trifluoromethyl, X² is | chlorodifluoromethyl | methoxy | Y¹ is CH, Y² is N, |
| | CH, X³ is trifluoromethyl | | | Y³ is CH |
| P.2457 | X¹ is trifluoromethyl, X² is | chlorodifluoromethyl | methyl | Y¹ is CH, Y² is N, |
| | CH, X³ is trifluoromethyl | | | Y³ is CH |
| P.2458 | X¹ is trifluoromethyl, X² is | chlorodifluoromethyl | nitro | Y¹ is CH, Y² is N, |
| | CH, X³ is trifluoromethyl | | | Y³ is CH |
| P.2459 | X¹ is trifluoromethyl, X² is | chlorodifluoromethyl | trifluoromethoxy | Y¹ is CH, Y² is N, |
| | CH, X³ is trifluoromethyl | | | Y³ is CH |
| P.2460 | X¹ is trifluoromethyl, X² is | chlorodifluoromethyl | trifluoromethyl | Y¹ is CH, Y² is N, |
| | CH, X³ is trifluoromethyl | | | Y³ is CH |
| P.2461 | X¹ is trifluoromethyl, X² is | difluoromethyl | bromo | Y¹ is CH, Y² is N, |
| | CH, X³ is trifluoromethyl | | | Y³ is CH |
| P.2462 | X¹ is trifluoromethyl, X² is | difluoromethyl | chloro | Y¹ is CH, Y² is N, |
| | CH, X³ is trifluoromethyl | | | Y³ is CH |
| P.2463 | X¹ is trifluoromethyl, X² is | difluoromethyl | cyano | Y¹ is CH, Y² is N, |
| | CH, X³ is trifluoromethyl | | | Y³ is CH |
| P.2464 | X¹ is trifluoromethyl, X² is | difluoromethyl | cyclopropyl | Y¹ is CH, Y² is N, |
| | CH, X³ is trifluoromethyl | | | Y³ is CH |
| P.2465 | X¹ is trifluoromethyl, X² is | difluoromethyl | ethyl | Y¹ is CH, Y² is N, |
| | CH, X³ is trifluoromethyl | | | Y³ is CH |
| P.2466 | X¹ is trifluoromethyl, X² is | difluoromethyl | fluoro | Y¹ is CH, Y² is N, |
| | CH, X³ is trifluoromethyl | | | Y³ is CH |
| P.2467 | X¹ is trifluoromethyl, X² is | difluoromethyl | hydrogen | Y¹ is CH, Y² is N, |
| | CH, X³ is trifluoromethyl | | | Y³ is CH |
| P.2468 | X¹ is trifluoromethyl, X² is | difluoromethyl | methoxy | Y¹ is CH, Y² is N, |
| | CH, X³ is trifluoromethyl | | | Y³ is CH |
| P.2469 | X¹ is trifluoromethyl, X² is | difluoromethyl | methyl | Y¹ is CH, Y² is N, |
| | CH, X³ is trifluoromethyl | | | Y³ is CH |
| P.2470 | X¹ is trifluoromethyl, X² is | difluoromethyl | nitro | Y¹ is CH, Y² is N, |
| | CH, X³ is trifluoromethyl | | | Y³ is CH |
| P.2471 | X¹ is trifluoromethyl, X² is | difluoromethyl | trifluoromethoxy | Y¹ is CH, Y² is N, |
| | CH, X³ is trifluoromethyl | | | Y³ is CH |
| P.2472 | X¹ is trifluoromethyl, X² is | difluoromethyl | trifluoromethyl | Y¹ is CH, Y² is N, |
| | CH, X³ is trifluoromethyl | | | Y³ is CH |
| P.2473 | X¹ is trifluoromethyl, X² is | trifluoromethyl | bromo | Y¹ is CH, Y² is N, |
| | CH, X³ is trifluoromethyl | | | Y³ is CH |
| P.2474 | X¹ is trifluoromethyl, X² is | trifluoromethyl | chloro | Y¹ is CH, Y² is N, |
| | CH, X³ is trifluoromethyl | | | Y³ is CH |
| P.2475 | X¹ is trifluoromethyl, X² is | trifluoromethyl | cyano | Y¹ is CH, Y² is N, |
| | CH, X³ is trifluoromethyl | | | Y³ is CH |
| P.2476 | X¹ is trifluoromethyl, X² is | trifluoromethyl | cyclopropyl | Y¹ is CH, Y² is N, |
| | CH, X³ is trifluoromethyl | | | Y³ is CH |
| P.2477 | X¹ is trifluoromethyl, X² is | trifluoromethyl | ethyl | Y¹ is CH, Y² is N, |
| | CH, X³ is trifluoromethyl | | | Y³ is CH |
| P.2478 | X¹ is trifluoromethyl, X² is | trifluoromethyl | fluoro | Y¹ is CH, Y² is N, |
| | CH, X³ is trifluoromethyl | | | Y³ is CH |
| P.2479 | X¹ is trifluoromethyl, X² is | trifluoromethyl | hydrogen | Y¹ is CH, Y² is N, |
| | CH, X³ is trifluoromethyl | | | Y³ is CH |
| P.2480 | X¹ is trifluoromethyl, X² is | trifluoromethyl | methoxy | Y¹ is CH, Y² is N, |
| | CH, X³ is trifluoromethyl | | | Y³ is CH |
| P.2481 | X¹ is trifluoromethyl, X² is | trifluoromethyl | methyl | Y¹ is CH, Y² is N, |
| | CH, X³ is trifluoromethyl | | | Y³ is CH |
| P.2482 | X¹ is trifluoromethyl, X² is | trifluoromethyl | nitro | Y¹ is CH, Y² is N, |
| | CH, X³ is trifluoromethyl | | | Y³ is CH |
| P.2483 | X¹ is trifluoromethyl, X² is | trifluoromethyl | trifluoromethoxy | Y¹ is CH, Y² is N, |
| | CH, X³ is trifluoromethyl | | | Y³ is CH |
| P.2484 | X¹ is trifluoromethyl, X² is | trifluoromethyl | trifluoromethyl | Y¹ is CH, Y² is N, |
| | CH, X³ is trifluoromethyl | | | Y³ is CH |
| P.2485 | X¹ is trifluoromethyl, X² is | chlorodifluoromethyl | bromo | Y¹ is CH, Y² is N, |
| | CH, X³ is hydrogen | | | Y³ is CH |
| P.2486 | X¹ is trifluoromethyl, X² is | chlorodifluoromethyl | chloro | Y¹ is CH, Y² is N, |
| | CH, X³ is hydrogen | | | Y³ is CH |
| P.2487 | X¹ is trifluoromethyl, X² is | chlorodifluoromethyl | cyano | Y¹ is CH, Y² is N, |
| | CH, X³ is hydrogen | | | Y³ is CH |
| P.2488 | X¹ is trifluoromethyl, X² is | chlorodifluoromethyl | cyclopropyl | Y¹ is CH, Y² is N, |
| | CH, X³ is hydrogen | | | Y³ is CH |
| P.2489 | X¹ is trifluoromethyl, X² is | chlorodifluoromethyl | ethyl | Y¹ is CH, Y² is N, |
| | CH, X³ is hydrogen | | | Y³ is CH |
| P.2490 | X¹ is trifluoromethyl, X² is | chlorodifluoromethyl | fluoro | Y¹ is CH, Y² is N, |
| | CH, X³ is hydrogen | | | Y³ is CH |
| P.2491 | X¹ is trifluoromethyl, X² is | chlorodifluoromethyl | hydrogen | Y¹ is CH, Y² is N, |
| | CH, X³ is hydrogen | | | Y³ is CH |
| P.2492 | X¹ is trifluoromethyl, X² is | chlorodifluoromethyl | methoxy | Y¹ is CH, Y² is N, |
| | CH, X³ is hydrogen | | | Y³ is CH |
| P.2493 | X¹ is trifluoromethyl, X² is | chlorodifluoromethyl | methyl | Y¹ is CH, Y² is N, |
| | CH, X³ is hydrogen | | | Y³ is CH |
| P.2494 | X¹ is trifluoromethyl, X² is | chlorodifluoromethyl | nitro | Y¹ is CH, Y² is N, |
| | CH, X³ is hydrogen | | | Y³ is CH |
| P.2495 | X¹ is trifluoromethyl, X² is | chlorodifluoromethyl | trifluoromethoxy | Y¹ is CH, Y² is N, |
| | CH, X³ is hydrogen | | | Y³ is CH |
| P.2496 | X¹ is trifluoromethyl, X² is | chlorodifluoromethyl | trifluoromethyl | Y¹ is CH, Y² is N, |
| | CH, X³ is hydrogen | | | Y³ is CH |
| P.2497 | X¹ is trifluoromethyl, X² is | difluoromethyl | bromo | Y¹ is CH, Y² is N, |
| | CH, X³ is hydrogen | | | Y³ is CH |
| P.2498 | X¹ is trifluoromethyl, X² is | difluoromethyl | chloro | Y¹ is CH, Y² is N, |
| | CH, X³ is hydrogen | | | Y³ is CH |
| P.2499 | X¹ is trifluoromethyl, X² is | difluoromethyl | cyano | Y¹ is CH, Y² is N, |
| | CH, X³ is hydrogen | | | Y³ is CH |
| P.2500 | X¹ is trifluoromethyl, X² is | difluoromethyl | cyclopropyl | Y¹ is CH, Y² is N, |
| | CH, X³ is hydrogen | | | Y³ is CH |
| P.2501 | X¹ is trifluoromethyl, X² is | difluoromethyl | ethyl | Y¹ is CH, Y² is N, |
| | CH, X³ is hydrogen | | | Y³ is CH |
| P.2502 | X¹ is trifluoromethyl, X² is | difluoromethyl | fluoro | Y¹ is CH, Y² is N, |
| | CH, X³ is hydrogen | | | Y³ is CH |
| P.2503 | X¹ is trifluoromethyl, X² is | difluoromethyl | hydrogen | Y¹ is CH, Y² is N, |
| | CH, X³ is hydrogen | | | Y³ is CH |
| P.2504 | X¹ is trifluoromethyl, X² is | difluoromethyl | methoxy | Y¹ is CH, Y² is N, |
| | CH, X³ is hydrogen | | | Y³ is CH |
| P.2505 | X¹ is trifluoromethyl, X² is | difluoromethyl | methyl | Y¹ is CH, Y² is N, |
| | CH, X³ is hydrogen | | | Y³ is CH |
| P.2506 | X¹ is trifluoromethyl, X² is | difluoromethyl | nitro | Y¹ is CH, Y² is N, |
| | CH, X³ is hydrogen | | | Y³ is CH |
| P.2507 | X¹ is trifluoromethyl, X² is | difluoromethyl | trifluoromethoxy | Y¹ is CH, Y² is N, |
| | CH, X³ is hydrogen | | | Y³ is CH |
| P.2508 | X¹ is trifluoromethyl, X² is | difluoromethyl | trifluoromethyl | Y¹ is CH, Y² is N, |
| | CH, X³ is hydrogen | | | Y³ is CH |
| P.2509 | X¹ is trifluoromethyl, X² is | trifluoromethyl | bromo | Y¹ is CH, Y² is N, |
| | CH, X³ is hydrogen | | | Y³ is CH |
| P.2510 | X¹ is trifluoromethyl, X² is | trifluoromethyl | chloro | Y¹ is CH, Y² is N, |
| | CH, X³ is hydrogen | | | Y³ is CH |
| P.2511 | X¹ is trifluoromethyl, X² is | trifluoromethyl | cyano | Y¹ is CH, Y² is N, |
| | CH, X³ is hydrogen | | | Y³ is CH |
| P.2512 | X¹ is trifluoromethyl, X² is | trifluoromethyl | cyclopropyl | Y¹ is CH, Y² is N, |
| | CH, X³ is hydrogen | | | Y³ is CH |
| P.2513 | X¹ is trifluoromethyl, X² is | trifluoromethyl | ethyl | Y¹ is CH, Y² is N, |
| | CH, X³ is hydrogen | | | Y³ is CH |
| P.2514 | X¹ is trifluoromethyl, X² is | trifluoromethyl | fluoro | Y¹ is CH, Y² is N, |
| | CH, X³ is hydrogen | | | Y³ is CH |
| P.2515 | X¹ is trifluoromethyl, X² is | trifluoromethyl | hydrogen | Y¹ is CH, Y² is N, |
| | CH, X³ is hydrogen | | | Y³ is CH |
| P.2516 | X¹ is trifluoromethyl, X² is | trifluoromethyl | methoxy | Y¹ is CH, Y² is N, |
| | CH, X³ is hydrogen | | | Y³ is CH |
| P.2517 | X¹ is trifluoromethyl, X² is | trifluoromethyl | methyl | Y¹ is CH, Y² is N, |
| | CH, X³ is hydrogen | | | Y³ is CH |
| P.2518 | X¹ is trifluoromethyl, X² is | trifluoromethyl | nitro | Y¹ is CH, Y² is N, |
| | CH, X³ is hydrogen | | | Y³ is CH |
| P.2519 | X¹ is trifluoromethyl, X² is | trifluoromethyl | trifluoromethoxy | Y¹ is CH, Y² is N, |
| | CH, X³ is hydrogen | | | Y³ is CH |
| P.2520 | X¹ is trifluoromethyl, X² is | trifluoromethyl | trifluoromethyl | Y¹ is CH, Y² is N, |
| | CH, X³ is hydrogen | | | Y³ is CH |
| P.2521 | X¹ is chloro, X² is N, X³ is | chlorodifluoromethyl | bromo | Y¹ is CH, Y² is N, |
| | chloro | | | Y³ is CH |
| P.2522 | X¹ is chloro, X² is N, X³ is | chlorodifluoromethyl | chloro | Y¹ is CH, Y² is N, |
| | chloro | | | Y³ is CH |
| P.2523 | X¹ is chloro, X² is N, X³ is | chlorodifluoromethyl | cyano | Y¹ is CH, Y² is N, |
| | chloro | | | Y³ is CH |
| P.2524 | X¹ is chloro, X² is N, X³ is | chlorodifluoromethyl | cyclopropyl | Y¹ is CH, Y² is N, |
| | chloro | | | Y³ is CH |
| P.2525 | X¹ is chloro, X² is N, X³ is | chlorodifluoromethyl | ethyl | Y¹ is CH, Y² is N, |
| | chloro | | | Y³ is CH |
| P.2526 | X¹ is chloro, X² is N, X³ is | chlorodifluoromethyl | fluoro | Y¹ is CH, Y² is N, |
| | chloro | | | Y³ is CH |
| P.2527 | X¹ is chloro, X² is N, X³ is | chlorodifluoromethyl | hydrogen | Y¹ is CH, Y² is N, |
| | chloro | | | Y³ is CH |
| P.2528 | X¹ is chloro, X² is N, X³ is | chlorodifluoromethyl | methoxy | Y¹ is CH, Y² is N, |
| | chloro | | | Y³ is CH |
| P.2529 | X¹ is chloro, X² is N, X³ is | chlorodifluoromethyl | methyl | Y¹ is CH, Y² is N, |
| | chloro | | | Y³ is CH |
| P.2530 | X¹ is chloro, X² is N, X³ is | chlorodifluoromethyl | nitro | Y¹ is CH, Y² is N, |
| | chloro | | | Y³ is CH |
| P.2531 | X¹ is chloro, X² is N, X³ is | chlorodifluoromethyl | trifluoromethoxy | Y¹ is CH, Y² is N, |
| | chloro | | | Y³ is CH |
| P.2532 | X¹ is chloro, X² is N, X³ is | chlorodifluoromethyl | trifluoromethyl | Y¹ is CH, Y² is N, |
| | chloro | | | Y³ is CH |
| P.2533 | X¹ is chloro, X² is N, X³ is | difluoromethyl | bromo | Y¹ is CH, Y² is N, |
| | chloro | | | Y³ is CH |
| P.2534 | X¹ is chloro, X² is N, X³ is | difluoromethyl | chloro | Y¹ is CH, Y² is N, |
| | chloro | | | Y³ is CH |
| P.2535 | X¹ is chloro, X² is N, X³ is | difluoromethyl | cyano | Y¹ is CH, Y² is N, |
| | chloro | | | Y³ is CH |
| P.2536 | X¹ is chloro, X² is N, X³ is | difluoromethyl | cyclopropyl | Y¹ is CH, Y² is N, |
| | chloro | | | Y³ is CH |
| P.2537 | X¹ is chloro, X² is N, X³ is | difluoromethyl | ethyl | Y¹ is CH, Y² is N, |
| | chloro | | | Y³ is CH |
| P.2538 | X¹ is chloro, X² is N, X³ is | difluoromethyl | fluoro | Y¹ is CH, Y² is N, |
| | chloro | | | Y³ is CH |
| P.2539 | X¹ is chloro, X² is N, X³ is | difluoromethyl | hydrogen | Y¹ is CH, Y² is N, |
| | chloro | | | Y³ is CH |
| P.2540 | X¹ is chloro, X² is N, X³ is | difluoromethyl | methoxy | Y¹ is CH, Y² is N, |
| | chloro | | | Y³ is CH |
| P.2541 | X¹ is chloro, X² is N, X³ is | difluoromethyl | methyl | Y¹ is CH, Y² is N, |
| | chloro | | | Y³ is CH |
| P.2542 | X¹ is chloro, X² is N, X³ is | difluoromethyl | nitro | Y¹ is CH, Y² is N, |
| | chloro | | | Y³ is CH |
| P.2543 | X¹ is chloro, X² is N, X³ is | difluoromethyl | trifluoromethoxy | Y¹ is CH, Y² is N, |
| | chloro | | | Y³ is CH |
| P.2544 | X¹ is chloro, X² is N, X³ is | difluoromethyl | trifluoromethyl | Y¹ is CH, Y² is N, |
| | chloro | | | Y³ is CH |
| P.2545 | X¹ is chloro, X² is N, X³ is | trifluoromethyl | bromo | Y¹ is CH, Y² is N, |
| | chloro | | | Y³ is CH |
| P.2546 | X¹ is chloro, X² is N, X³ is | trifluoromethyl | chloro | Y¹ is CH, Y² is N, |
| | chloro | | | Y³ is CH |
| P.2547 | X¹ is chloro, X² is N, X³ is | trifluoromethyl | cyano | Y¹ is CH, Y² is N, |
| | chloro | | | Y³ is CH |
| P.2548 | X¹ is chloro, X² is N, X³ is | trifluoromethyl | cyclopropyl | Y¹ is CH, Y² is N, |
| | chloro | | | Y³ is CH |
| P.2549 | X¹ is chloro, X² is N, X³ is | trifluoromethyl | ethyl | Y¹ is CH, Y² is N, |
| | chloro | | | Y³ is CH |
| P.2550 | X¹ is chloro, X² is N, X³ is | trifluoromethyl | fluoro | Y¹ is CH, Y² is N, |
| | chloro | | | Y³ is CH |
| P.2551 | X¹ is chloro, X² is N, X³ is | trifluoromethyl | hydrogen | Y¹ is CH, Y² is N, |
| | chloro | | | Y³ is CH |
| P.2552 | X¹ is chloro, X² is N, X³ is | trifluoromethyl | methoxy | Y¹ is CH, Y² is N, |
| | chloro | | | Y³ is CH |
| P.2553 | X¹ is chloro, X² is N, X³ is | trifluoromethyl | methyl | Y¹ is CH, Y² is N, |
| | chloro | | | Y³ is CH |
| P.2554 | X¹ is chloro, X² is N, X³ is | trifluoromethyl | nitro | Y¹ is CH, Y² is N, |
| | chloro | | | Y³ is CH |
| P.2555 | X¹ is chloro, X² is N, X³ is | trifluoromethyl | trifluoromethoxy | Y¹ is CH, Y² is N, |
| | chloro | | | Y³ is CH |
| P.2556 | X¹ is chloro, X² is N, X³ is | trifluoromethyl | trifluoromethyl | Y¹ is CH, Y² is N, |
| | chloro | | | Y³ is CH |
| P.2557 | X¹ is trifluoromethyl, X² is | chlorodifluoromethyl | bromo | Y¹ is CH, Y² is N, |
| | N, X³ is trifluoromethyl | | | Y³ is CH |
| P.2558 | X¹ is trifluoromethyl, X² is | chlorodifluoromethyl | chloro | Y¹ is CH, Y² is N, |
| | N, X³ is trifluoromethyl | | | Y³ is CH |
| P.2559 | X¹ is trifluoromethyl, X² is | chlorodifluoromethyl | cyano | Y¹ is CH, Y² is N, |
| | N, X³ is trifluoromethyl | | | Y³ is CH |
| P.2560 | X¹ is trifluoromethyl, X² is | chlorodifluoromethyl | cyclopropyl | Y¹ is CH, Y² is N, |
| | N, X³ is trifluoromethyl | | | Y³ is CH |
| P.2561 | X¹ is trifluoromethyl, X² is | chlorodifluoromethyl | ethyl | Y¹ is CH, Y² is N, |
| | N, X³ is trifluoromethyl | | | Y³ is CH |
| P.2562 | X¹ is trifluoromethyl, X² is | chlorodifluoromethyl | fluoro | Y¹ is CH, Y² is N, |
| | N, X³ is trifluoromethyl | | | Y³ is CH |
| P.2563 | X¹ is trifluoromethyl, X² is | chlorodifluoromethyl | hydrogen | Y¹ is CH, Y² is N, |
| | N, X³ is trifluoromethyl | | | Y³ is CH |
| P.2564 | X¹ is trifluoromethyl, X² is | chlorodifluoromethyl | methoxy | Y¹ is CH, Y² is N, |
| | N, X³ is trifluoromethyl | | | Y³ is CH |
| P.2565 | X¹ is trifluoromethyl, X² is | chlorodifluoromethyl | methyl | Y¹ is CH, Y² is N, |
| | N, X³ is trifluoromethyl | | | Y³ is CH |
| P.2566 | X¹ is trifluoromethyl, X² is | chlorodifluoromethyl | nitro | Y¹ is CH, Y² is N, |
| | N, X³ is trifluoromethyl | | | Y³ is CH |
| P.2567 | X¹ is trifluoromethyl, X² is | chlorodifluoromethyl | trifluoromethoxy | Y¹ is CH, Y² is N, |
| | N, X³ is trifluoromethyl | | | Y³ is CH |
| P.2568 | X¹ is trifluoromethyl, X² is | chlorodifluoromethyl | trifluoromethyl | Y¹ is CH, Y² is N, |
| | N, X³ is trifluoromethyl | | | Y³ is CH |
| P.2569 | X¹ is trifluoromethyl, X² is | difluoromethyl | bromo | Y¹ is CH, Y² is N, |
| | N, X³ is trifluoromethyl | | | Y³ is CH |
| P.2570 | X¹ is trifluoromethyl, X² is | difluoromethyl | chloro | Y¹ is CH, Y² is N, |
| | N, X³ is trifluoromethyl | | | Y³ is CH |
| P.2571 | X¹ is trifluoromethyl, X² is | difluoromethyl | cyano | Y¹ is CH, Y² is N, |
| | N, X³ is trifluoromethyl | | | Y³ is CH |
| P.2572 | X¹ is trifluoromethyl, X² is | difluoromethyl | cyclopropyl | Y¹ is CH, Y² is N, |
| | N, X³ is trifluoromethyl | | | Y³ is CH |
| P.2573 | X¹ is trifluoromethyl, X² is | difluoromethyl | ethyl | Y¹ is CH, Y² is N, |
| | N, X³ is trifluoromethyl | | | Y³ is CH |
| P.2574 | X¹ is trifluoromethyl, X² is | difluoromethyl | fluoro | Y¹ is CH, Y² is N, |
| | N, X³ is trifluoromethyl | | | Y³ is CH |
| P.2575 | X¹ is trifluoromethyl, X² is | difluoromethyl | hydrogen | Y¹ is CH, Y² is N, |
| | N, X³ is trifluoromethyl | | | Y³ is CH |
| P.2576 | X¹ is trifluoromethyl, X² is | difluoromethyl | methoxy | Y¹ is CH, Y² is N, |
| | N, X³ is trifluoromethyl | | | Y³ is CH |
| P.2577 | X¹ is trifluoromethyl, X² is | difluoromethyl | methyl | Y¹ is CH, Y² is N, |
| | N, X³ is trifluoromethyl | | | Y³ is CH |
| P.2578 | X¹ is trifluoromethyl, X² is | difluoromethyl | nitro | Y¹ is CH, Y² is N, |
| | N, X³ is trifluoromethyl | | | Y³ is CH |
| P.2579 | X¹ is trifluoromethyl, X² is | difluoromethyl | trifluoromethoxy | Y¹ is CH, Y² is N, |
| | N, X³ is trifluoromethyl | | | Y³ is CH |
| P.2580 | X¹ is trifluoromethyl, X² is | difluoromethyl | trifluoromethyl | Y¹ is CH, Y² is N, |
| | N, X³ is trifluoromethyl | | | Y³ is CH |
| P.2581 | X¹ is trifluoromethyl, X² is | trifluoromethyl | bromo | Y¹ is CH, Y² is N, |
| | N, X³ is trifluoromethyl | | | Y³ is CH |
| P.2582 | X¹ is trifluoromethyl, X² is | trifluoromethyl | chloro | Y¹ is CH, Y² is N, |
| | N, X³ is trifluoromethyl | | | Y³ is CH |
| P.2583 | X¹ is trifluoromethyl, X² is | trifluoromethyl | cyano | Y¹ is CH, Y² is N, |
| | N, X³ is trifluoromethyl | | | Y³ is CH |
| P.2584 | X¹ is trifluoromethyl, X² is | trifluoromethyl | cyclopropyl | Y¹ is CH, Y² is N, |
| | N, X³ is trifluoromethyl | | | Y³ is CH |
| P.2585 | X¹ is trifluoromethyl, X² is | trifluoromethyl | ethyl | Y¹ is CH, Y² is N, |
| | N, X³ is trifluoromethyl | | | Y³ is CH |
| P.2586 | X¹ is trifluoromethyl, X² is | trifluoromethyl | fluoro | Y¹ is CH, Y² is N, |
| | N, X³ is trifluoromethyl | | | Y³ is CH |
| P.2587 | X¹ is trifluoromethyl, X² is | trifluoromethyl | hydrogen | Y¹ is CH, Y² is N, |
| | N, X³ is trifluoromethyl | | | Y³ is CH |
| P.2588 | X¹ is trifluoromethyl, X² is | trifluoromethyl | methoxy | Y¹ is CH, Y² is N, |
| | N, X³ is trifluoromethyl | | | Y³ is CH |
| P.2589 | X¹ is trifluoromethyl, X² is | trifluoromethyl | methyl | Y¹ is CH, Y² is N, |
| | N, X³ is trifluoromethyl | | | Y³ is CH |
| P.2590 | X¹ is trifluoromethyl, X² is | trifluoromethyl | nitro | Y¹ is CH, Y² is N, |
| | N, X³ is trifluoromethyl | | | Y³ is CH |
| P.2591 | X¹ is trifluoromethyl, X² is | trifluoromethyl | trifluoromethoxy | Y¹ is CH, Y² is N, |
| | N, X³ is trifluoromethyl | | | Y³ is CH |
| P.2592 | X¹ is trifluoromethyl, X² is | trifluoromethyl | trifluoromethyl | Y¹ is CH, Y² is N, |
| | N, X³ is trifluoromethyl | | | Y³ is CH |
| P.2593 | X¹ is chloro, X² is CH, X³ is | chlorodifluoromethyl | bromo | Y¹ is CH, Y² is |
| | chloro | | | CH, Y³ is N |
| P.2594 | X¹ is chloro, X² is CH, X³ is | chlorodifluoromethyl | chloro | Y¹ is CH, Y² is |
| | chloro | | | CH, Y³ is N |
| P.2595 | X¹ is chloro, X² is CH, X³ is | chlorodifluoromethyl | cyano | Y¹ is CH, Y² is |
| | chloro | | | CH, Y³ is N |
| P.2596 | X¹ is chloro, X² is CH, X³ is | chlorodifluoromethyl | cyclopropyl | Y¹ is CH, Y² is |
| | chloro | | | CH, Y³ is N |
| P.2597 | X¹ is chloro, X² is CH, X³ is | chlorodifluoromethyl | ethyl | Y¹ is CH, Y² is |
| | chloro | | | CH, Y³ is N |
| P.2598 | X¹ is chloro, X² is CH, X³ is | chlorodifluoromethyl | fluoro | Y¹ is CH, Y² is |
| | chloro | | | CH, Y³ is N |
| P.2599 | X¹ is chloro, X² is CH, X³ is | chlorodifluoromethyl | hydrogen | Y¹ is CH, Y² is |
| | chloro | | | CH, Y³ is N |
| P.2600 | X¹ is chloro, X² is CH, X³ is | chlorodifluoromethyl | methoxy | Y¹ is CH, Y² is |
| | chloro | | | CH, Y³ is N |
| P.2601 | X¹ is chloro, X² is CH, X³ is | chlorodifluoromethyl | methyl | Y¹ is CH, Y² is |
| | chloro | | | CH, Y³ is N |
| P.2602 | X¹ is chloro, X² is CH, X³ is | chlorodifluoromethyl | nitro | Y¹ is CH, Y² is |
| | chloro | | | CH, Y³ is N |
| P.2603 | X¹ is chloro, X² is CH, X³ is | chlorodifluoromethyl | trifluoromethoxy | Y¹ is CH, Y² is |
| | chloro | | | CH, Y³ is N |
| P.2604 | X¹ is chloro, X² is CH, X³ is | chlorodifluoromethyl | trifluoromethyl | Y¹ is CH, Y² is |
| | chloro | | | CH, Y³ is N |
| P.2605 | X¹ is chloro, X² is CH, X³ is | difluoromethyl | bromo | Y¹ is CH, Y² is |
| | chloro | | | CH, Y³ is N |
| P.2606 | X¹ is chloro, X² is CH, X³ is | difluoromethyl | chloro | Y¹ is CH, Y² is |
| | chloro | | | CH, Y³ is N |
| P.2607 | X¹ is chloro, X² is CH, X³ is | difluoromethyl | cyano | Y¹ is CH, Y² is |
| | chloro | | | CH, Y³ is N |
| P.2608 | X¹ is chloro, X² is CH, X³ is | difluoromethyl | cyclopropyl | Y¹ is CH, Y² is |
| | chloro | | | CH, Y³ is N |
| P.2609 | X¹ is chloro, X² is CH, X³ is | difluoromethyl | ethyl | Y¹ is CH, Y² is |
| | chloro | | | CH, Y³ is N |
| P.2610 | X¹ is chloro, X² is CH, X³ is | difluoromethyl | fluoro | Y¹ is CH, Y² is |
| | chloro | | | CH, Y³ is N |
| P.2611 | X¹ is chloro, X² is CH, X³ is | difluoromethyl | hydrogen | Y¹ is CH, Y² is |
| | chloro | | | CH, Y³ is N |
| P.2612 | X¹ is chloro, X² is CH, X³ is | difluoromethyl | methoxy | Y¹ is CH, Y² is |
| | chloro | | | CH, Y³ is N |
| P.2613 | X¹ is chloro, X² is CH, X³ is | difluoromethyl | methyl | Y¹ is CH, Y² is |
| | chloro | | | CH, Y³ is N |
| P.2614 | X¹ is chloro, X² is CH, X³ is | difluoromethyl | nitro | Y¹ is CH, Y² is |
| | chloro | | | CH, Y³ is N |
| P.2615 | X¹ is chloro, X² is CH, X³ is | difluoromethyl | trifluoromethoxy | Y¹ is CH, Y² is |
| | chloro | | | CH, Y³ is N |
| P.2616 | X¹ is chloro, X² is CH, X³ is | difluoromethyl | trifluoromethyl | Y¹ is CH, Y² is |
| | chloro | | | CH, Y³ is N |
| P.2617 | X¹ is chloro, X² is CH, X³ is | trifluoromethyl | bromo | Y¹ is CH, Y² is |
| | chloro | | | CH, Y³ is N |
| P.2618 | X¹ is chloro, X² is CH, X³ is | trifluoromethyl | chloro | Y¹ is CH, Y² is |
| | chloro | | | CH, Y³ is N |
| P.2619 | X¹ is chloro, X² is CH, X³ is | trifluoromethyl | cyano | Y¹ is CH, Y² is |
| | chloro | | | CH, Y³ is N |
| P.2620 | X¹ is chloro, X² is CH, X³ is | trifluoromethyl | cyclopropyl | Y¹ is CH, Y² is |
| | chloro | | | CH, Y³ is N |
| P.2621 | X¹ is chloro, X² is CH, X³ is | trifluoromethyl | ethyl | Y¹ is CH, Y² is |
| | chloro | | | CH, Y³ is N |
| P.2622 | X¹ is chloro, X² is CH, X³ is | trifluoromethyl | fluoro | Y¹ is CH, Y² is |
| | chloro | | | CH, Y³ is N |
| P.2623 | X¹ is chloro, X² is CH, X³ is | trifluoromethyl | hydrogen | Y¹ is CH, Y² is |
| | chloro | | | CH, Y³ is N |
| P.2624 | X¹ is chloro, X² is CH, X³ is | trifluoromethyl | methoxy | Y¹ is CH, Y² is |
| | chloro | | | CH, Y³ is N |
| P.2625 | X¹ is chloro, X² is CH, X³ is | trifluoromethyl | methyl | Y¹ is CH, Y² is |
| | chloro | | | CH, Y³ is N |
| P.2626 | X¹ is chloro, X² is CH, X³ is | trifluoromethyl | nitro | Y¹ is CH, Y² is |
| | chloro | | | CH, Y³ is N |
| P.2627 | X¹ is chloro, X² is CH, X³ is | trifluoromethyl | trifluoromethoxy | Y¹ is CH, Y² is |
| | chloro | | | CH, Y³ is N |
| P.2628 | X¹ is chloro, X² is CH, X³ is | trifluoromethyl | trifluoromethyl | Y¹ is CH, Y² is |
| | chloro | | | CH, Y³ is N |
| P.2629 | X¹ is chloro, X² is C—F, X³ is | chlorodifluoromethyl | bromo | Y¹ is CH, Y² is |
| | hydrogen | | | CH, Y³ is N |
| P.2630 | X¹ is chloro, X² is C—F, X³ is | chlorodifluoromethyl | chloro | Y¹ is CH, Y² is |
| | hydrogen | | | CH, Y³ is N |
| P.2631 | X¹ is chloro, X² is C—F, X³ is | chlorodifluoromethyl | cyano | Y¹ is CH, Y² is |
| | hydrogen | | | CH, Y³ is N |
| P.2632 | X¹ is chloro, X² is C—F, X³ is | chlorodifluoromethyl | cyclopropyl | Y¹ is CH, Y² is |
| | hydrogen | | | CH, Y³ is N |
| P.2633 | X¹ is chloro, X² is C—F, X³ is | chlorodifluoromethyl | ethyl | Y¹ is CH, Y² is |
| | hydrogen | | | CH, Y³ is N |
| P.2634 | X¹ is chloro, X² is C—F, X³ is | chlorodifluoromethyl | fluoro | Y¹ is CH, Y² is |
| | hydrogen | | | CH, Y³ is N |
| P.2635 | X¹ is chloro, X² is C—F, X³ is | chlorodifluoromethyl | hydrogen | Y¹ is CH, Y² is |
| | hydrogen | | | CH, Y³ is N |
| P.2636 | X¹ is chloro, X² is C—F, X³ is | chlorodifluoromethyl | methoxy | Y¹ is CH, Y² is |
| | hydrogen | | | CH, Y³ is N |
| P.2637 | X¹ is chloro, X² is C—F, X³ is | chlorodifluoromethyl | methyl | Y¹ is CH, Y² is |
| | hydrogen | | | CH, Y³ is N |
| P.2638 | X¹ is chloro, X² is C—F, X³ is | chlorodifluoromethyl | nitro | Y¹ is CH, Y² is |
| | hydrogen | | | CH, Y³ is N |
| P.2639 | X¹ is chloro, X² is C—F, X³ is | chlorodifluoromethyl | trifluoromethoxy | Y¹ is CH, Y² is |
| | hydrogen | | | CH, Y³ is N |
| P.2640 | X¹ is chloro, X² is C—F, X³ is | chlorodifluoromethyl | trifluoromethyl | Y¹ is CH, Y² is |
| | hydrogen | | | CH, Y³ is N |
| P.2641 | X¹ is chloro, X² is C—F, X³ is | difluoromethyl | bromo | Y¹ is CH, Y² is |
| | hydrogen | | | CH, Y³ is N |
| P.2642 | X¹ is chloro, X² is C—F, X³ is | difluoromethyl | chloro | Y¹ is CH, Y² is |
| | hydrogen | | | CH, Y³ is N |
| P.2643 | X¹ is chloro, X² is C—F, X³ is | difluoromethyl | cyano | Y¹ is CH, Y² is |
| | hydrogen | | | CH, Y³ is N |
| P.2644 | X¹ is chloro, X² is C—F, X³ is | difluoromethyl | cyclopropyl | Y¹ is CH, Y² is |
| | hydrogen | | | CH, Y³ is N |
| P.2645 | X¹ is chloro, X² is C—F, X³ is | difluoromethyl | ethyl | Y¹ is CH, Y² is |
| | hydrogen | | | CH, Y³ is N |
| P.2646 | X¹ is chloro, X² is C—F, X³ is | difluoromethyl | fluoro | Y¹ is CH, Y² is |
| | hydrogen | | | CH, Y³ is N |
| P.2647 | X¹ is chloro, X² is C—F, X³ is | difluoromethyl | hydrogen | Y¹ is CH, Y² is |
| | hydrogen | | | CH, Y³ is N |
| P.2648 | X¹ is chloro, X² is C—F, X³ is | difluoromethyl | methoxy | Y¹ is CH, Y² is |
| | hydrogen | | | CH, Y³ is N |
| P.2649 | X¹ is chloro, X² is C—F, X³ is | difluoromethyl | methyl | Y¹ is CH, Y² is |
| | hydrogen | | | CH, Y³ is N |
| P.2650 | X¹ is chloro, X² is C—F, X³ is | difluoromethyl | nitro | Y¹ is CH, Y² is |
| | hydrogen | | | CH, Y³ is N |
| P.2651 | X¹ is chloro, X² is C—F, X³ is | difluoromethyl | trifluoromethoxy | Y¹ is CH, Y² is |
| | hydrogen | | | CH, Y³ is N |
| P.2652 | X¹ is chloro, X² is C—F, X³ is | difluoromethyl | trifluoromethyl | Y¹ is CH, Y² is |
| | hydrogen | | | CH, Y³ is N |
| P.2653 | X¹ is chloro, X² is C—F, X³ is | trifluoromethyl | bromo | Y¹ is CH, Y² is |
| | hydrogen | | | CH, Y³ is N |
| P.2654 | X¹ is chloro, X² is C—F, X³ is | trifluoromethyl | chloro | Y¹ is CH, Y² is |
| | hydrogen | | | CH, Y³ is N |
| P.2655 | X¹ is chloro, X² is C—F, X³ is | trifluoromethyl | cyano | Y¹ is CH, Y² is |
| | hydrogen | | | CH, Y³ is N |
| P.2656 | X¹ is chloro, X² is C—F, X³ is | trifluoromethyl | cyclopropyl | Y¹ is CH, Y² is |
| | hydrogen | | | CH, Y³ is N |
| P.2657 | X¹ is chloro, X² is C—F, X³ is | trifluoromethyl | ethyl | Y¹ is CH, Y² is |
| | hydrogen | | | CH, Y³ is N |
| P.2658 | X¹ is chloro, X² is C—F, X³ is | trifluoromethyl | fluoro | Y¹ is CH, Y² is |
| | hydrogen | | | CH, Y³ is N |
| P.2659 | X¹ is chloro, X² is C—F, X³ is | trifluoromethyl | hydrogen | Y¹ is CH, Y² is |
| | hydrogen | | | CH, Y³ is N |
| P.2660 | X¹ is chloro, X² is C—F, X³ is | trifluoromethyl | methoxy | Y¹ is CH, Y² is |
| | hydrogen | | | CH, Y³ is N |
| P.2661 | X¹ is chloro, X² is C—F, X³ is | trifluoromethyl | methyl | Y¹ is CH, Y² is |
| | hydrogen | | | CH, Y³ is N |
| P.2662 | X¹ is chloro, X² is C—F, X³ is | trifluoromethyl | nitro | Y¹ is CH, Y² is |
| | hydrogen | | | CH, Y³ is N |
| P.2663 | X¹ is chloro, X² is C—F, X³ is | trifluoromethyl | trifluoromethoxy | Y¹ is CH, Y² is |
| | hydrogen | | | CH, Y³ is N |
| P.2664 | X¹ is chloro, X² is C—F, X³ is | trifluoromethyl | trifluoromethyl | Y¹ is CH, Y² is |
| | hydrogen | | | CH, Y³ is N |
| P.2665 | X¹ is fluoro, X² is C—Cl, X³ | chlorodifluoromethyl | bromo | Y¹ is CH, Y² is |
| | is hydrogen | | | CH, Y³ is N |
| P.2666 | X¹ is fluoro, X² is C—Cl, X³ | chlorodifluoromethyl | chloro | Y¹ is CH, Y² is |
| | is hydrogen | | | CH, Y³ is N |
| P.2667 | X¹ is fluoro, X² is C—Cl, X³ | chlorodifluoromethyl | cyano | Y¹ is CH, Y² is |
| | is hydrogen | | | CH, Y³ is N |
| P.2668 | X¹ is fluoro, X² is C—Cl, X³ | chlorodifluoromethyl | cyclopropyl | Y¹ is CH, Y² is |
| | is hydrogen | | | CH, Y³ is N |
| P.2669 | X¹ is fluoro, X² is C—Cl, X³ | chlorodifluoromethyl | ethyl | Y¹ is CH, Y² is |
| | is hydrogen | | | CH, Y³ is N |
| P.2670 | X¹ is fluoro, X² is C—Cl, X³ | chlorodifluoromethyl | fluoro | Y¹ is CH, Y² is |
| | is hydrogen | | | CH, Y³ is N |
| P.2671 | X¹ is fluoro, X² is C—Cl, X³ | chlorodifluoromethyl | hydrogen | Y¹ is CH, Y² is |
| | is hydrogen | | | CH, Y³ is N |
| P.2672 | X¹ is fluoro, X² is C—Cl, X³ | chlorodifluoromethyl | methoxy | Y¹ is CH, Y² is |
| | is hydrogen | | | CH, Y³ is N |
| P.2673 | X¹ is fluoro, X² is C—Cl, X³ | chlorodifluoromethyl | methyl | Y¹ is CH, Y² is |
| | is hydrogen | | | CH, Y³ is N |
| P.2674 | X¹ is fluoro, X² is C—Cl, X³ | chlorodifluoromethyl | nitro | Y¹ is CH, Y² is |
| | is hydrogen | | | CH, Y³ is N |
| P.2675 | X¹ is fluoro, X² is C—Cl, X³ | chlorodifluoromethyl | trifluoromethoxy | Y¹ is CH, Y² is |
| | is hydrogen | | | CH, Y³ is N |
| P.2676 | X¹ is fluoro, X² is C—Cl, X³ | chlorodifluoromethyl | trifluoromethyl | Y¹ is CH, Y² is |
| | is hydrogen | | | CH, Y³ is N |
| P.2677 | X¹ is fluoro, X² is C—Cl, X³ | difluoromethyl | bromo | Y¹ is CH, Y² is |
| | is hydrogen | | | CH, Y³ is N |
| P.2678 | X¹ is fluoro, X² is C—Cl, X³ | difluoromethyl | chloro | Y¹ is CH, Y² is |
| | is hydrogen | | | CH, Y³ is N |
| P.2679 | X¹ is fluoro, X² is C—Cl, X³ | difluoromethyl | cyano | Y¹ is CH, Y² is |
| | is hydrogen | | | CH, Y³ is N |
| P.2680 | X¹ is fluoro, X² is C—Cl, X³ | difluoromethyl | cyclopropyl | Y¹ is CH, Y² is |
| | is hydrogen | | | CH, Y³ is N |
| P.2681 | X¹ is fluoro, X² is C—Cl, X³ | difluoromethyl | ethyl | Y¹ is CH, Y² is |
| | is hydrogen | | | CH, Y³ is N |
| P.2682 | X¹ is fluoro, X² is C—Cl, X³ | difluoromethyl | fluoro | Y¹ is CH, Y² is |
| | is hydrogen | | | CH, Y³ is N |
| P.2683 | X¹ is fluoro, X² is C—Cl, X³ | difluoromethyl | hydrogen | Y¹ is CH, Y² is |
| | is hydrogen | | | CH, Y³ is N |
| P.2684 | X¹ is fluoro, X² is C—Cl, X³ | difluoromethyl | methoxy | Y¹ is CH, Y² is |
| | is hydrogen | | | CH, Y³ is N |
| P.2685 | X¹ is fluoro, X² is C—Cl, X³ | difluoromethyl | methyl | Y¹ is CH, Y² is |
| | is hydrogen | | | CH, Y³ is N |
| P.2686 | X¹ is fluoro, X² is C—Cl, X³ | difluoromethyl | nitro | Y¹ is CH, Y² is |
| | is hydrogen | | | CH, Y³ is N |
| P.2687 | X¹ is fluoro, X² is C—Cl, X³ | difluoromethyl | trifluoromethoxy | Y¹ is CH, Y² is |
| | is hydrogen | | | CH, Y³ is N |
| P.2688 | X¹ is fluoro, X² is C—Cl, X³ | difluoromethyl | trifluoromethyl | Y¹ is CH, Y² is |
| | is hydrogen | | | CH, Y³ is N |
| P.2689 | X¹ is fluoro, X² is C—Cl, X³ | trifluoromethyl | bromo | Y¹ is CH, Y² is |
| | is hydrogen | | | CH, Y³ is N |
| P.2690 | X¹ is fluoro, X² is C—Cl, X³ | trifluoromethyl | chloro | Y¹ is CH, Y² is |
| | is hydrogen | | | CH, Y³ is N |
| P.2691 | X¹ is fluoro, X² is C—Cl, X³ | trifluoromethyl | cyano | Y¹ is CH, Y² is |
| | is hydrogen | | | CH, Y³ is N |
| P.2692 | X¹ is fluoro, X² is C—Cl, X³ | trifluoromethyl | cyclopropyl | Y¹ is CH, Y² is |
| | is hydrogen | | | CH, Y³ is N |
| P.2693 | X¹ is fluoro, X² is C—Cl, X³ | trifluoromethyl | ethyl | Y¹ is CH, Y² is |
| | is hydrogen | | | CH, Y³ is N |
| P.2694 | X¹ is fluoro, X² is C—Cl, X³ | trifluoromethyl | fluoro | Y¹ is CH, Y² is |
| | is hydrogen | | | CH, Y³ is N |
| P.2695 | X¹ is fluoro, X² is C—Cl, X³ | trifluoromethyl | hydrogen | Y¹ is CH, Y² is |
| | is hydrogen | | | CH, Y³ is N |
| P.2696 | X¹ is fluoro, X² is C—Cl, X³ | trifluoromethyl | methoxy | Y¹ is CH, Y² is |
| | is hydrogen | | | CH, Y³ is N |
| P.2697 | X¹ is fluoro, X² is C—Cl, X³ | trifluoromethyl | methyl | Y¹ is CH, Y² is |
| | is hydrogen | | | CH, Y³ is N |
| P.2698 | X¹ is fluoro, X² is C—Cl, X³ | trifluoromethyl | nitro | Y¹ is CH, Y² is |
| | is hydrogen | | | CH, Y³ is N |
| P.2699 | X¹ is fluoro, X² is C—Cl, X³ | trifluoromethyl | trifluoromethoxy | Y¹ is CH, Y² is |
| | is hydrogen | | | CH, Y³ is N |
| P.2700 | X¹ is fluoro, X² is C—Cl, X³ | trifluoromethyl | trifluoromethyl | Y¹ is CH, Y² is |
| | is hydrogen | | | CH, Y³ is N |
| P.2701 | X¹ is chloro, X² is C—Cl, X³ | chlorodifluoromethyl | bromo | Y¹ is CH, Y² is |
| | is hydrogen | | | CH, Y³ is N |
| P.2702 | X¹ is chloro, X² is C—Cl, X³ | chlorodifluoromethyl | chloro | Y¹ is CH, Y² is |
| | is hydrogen | | | CH, Y³ is N |
| P.2703 | X¹ is chloro, X² is C—Cl, X³ | chlorodifluoromethyl | cyano | Y¹ is CH, Y² is |
| | is hydrogen | | | CH, Y³ is N |
| P.2704 | X¹ is chloro, X² is C—Cl, X³ | chlorodifluoromethyl | cyclopropyl | Y¹ is CH, Y² is |
| | is hydrogen | | | CH, Y³ is N |
| P.2705 | X¹ is chloro, X² is C—Cl, X³ | chlorodifluoromethyl | ethyl | Y¹ is CH, Y² is |
| | is hydrogen | | | CH, Y³ is N |
| P.2706 | X¹ is chloro, X² is C—Cl, X³ | chlorodifluoromethyl | fluoro | Y¹ is CH, Y² is |
| | is hydrogen | | | CH, Y³ is N |
| P.2707 | X¹ is chloro, X² is C—Cl, X³ | chlorodifluoromethyl | hydrogen | Y¹ is CH, Y² is |
| | is hydrogen | | | CH, Y³ is N |
| P.2708 | X¹ is chloro, X² is C—Cl, X³ | chlorodifluoromethyl | methoxy | Y¹ is CH, Y² is |
| | is hydrogen | | | CH, Y³ is N |
| P.2709 | X¹ is chloro, X² is C—Cl, X³ | chlorodifluoromethyl | methyl | Y¹ is CH, Y² is |
| | is hydrogen | | | CH, Y³ is N |
| P.2710 | X¹ is chloro, X² is C—Cl, X³ | chlorodifluoromethyl | nitro | Y¹ is CH, Y² is |
| | is hydrogen | | | CH, Y³ is N |
| P.2711 | X¹ is chloro, X² is C—Cl, X³ | chlorodifluoromethyl | trifluoromethoxy | Y¹ is CH, Y² is |
| | is hydrogen | | | CH, Y³ is N |
| P.2712 | X¹ is chloro, X² is C—Cl, X³ | chlorodifluoromethyl | trifluoromethyl | Y¹ is CH, Y² is |
| | is hydrogen | | | CH, Y³ is N |
| P.2713 | X¹ is chloro, X² is C—Cl, X³ | difluoromethyl | bromo | Y¹ is CH, Y² is |
| | is hydrogen | | | CH, Y³ is N |
| P.2714 | X¹ is chloro, X² is C—Cl, X³ | difluoromethyl | chloro | Y¹ is CH, Y² is |
| | is hydrogen | | | CH, Y³ is N |
| P.2715 | X¹ is chloro, X² is C—Cl, X³ | difluoromethyl | cyano | Y¹ is CH, Y² is |
| | is hydrogen | | | CH, Y³ is N |
| P.2716 | X¹ is chloro, X² is C—Cl, X³ | difluoromethyl | cyclopropyl | Y¹ is CH, Y² is |
| | is hydrogen | | | CH, Y³ is N |
| P.2717 | X¹ is chloro, X² is C—Cl, X³ | difluoromethyl | ethyl | Y¹ is CH, Y² is |
| | is hydrogen | | | CH, Y³ is N |
| P.2718 | X¹ is chloro, X² is C—Cl, X³ | difluoromethyl | fluoro | Y¹ is CH, Y² is |
| | is hydrogen | | | CH, Y³ is N |
| P.2719 | X¹ is chloro, X² is C—Cl, X³ | difluoromethyl | hydrogen | Y¹ is CH, Y² is |
| | is hydrogen | | | CH, Y³ is N |
| P.2720 | X¹ is chloro, X² is C—Cl, X³ | difluoromethyl | methoxy | Y¹ is CH, Y² is |
| | is hydrogen | | | CH, Y³ is N |
| P.2721 | X¹ is chloro, X² is C—Cl, X³ | difluoromethyl | methyl | Y¹ is CH, Y² is |
| | is hydrogen | | | CH, Y³ is N |
| P.2722 | X¹ is chloro, X² is C—Cl, X³ | difluoromethyl | nitro | Y¹ is CH, Y² is |
| | is hydrogen | | | CH, Y³ is N |
| P.2723 | X¹ is chloro, X² is C—Cl, X³ | difluoromethyl | trifluoromethoxy | Y¹ is CH, Y² is |
| | is hydrogen | | | CH, Y³ is N |
| P.2724 | X¹ is chloro, X² is C—Cl, X³ | difluoromethyl | trifluoromethyl | Y¹ is CH, Y² is |
| | is hydrogen | | | CH, Y³ is N |
| P.2725 | X¹ is chloro, X² is C—Cl, X³ | trifluoromethyl | bromo | Y¹ is CH, Y² is |
| | is hydrogen | | | CH, Y³ is N |
| P.2726 | X¹ is chloro, X² is C—Cl, X³ | trifluoromethyl | chloro | Y¹ is CH, Y² is |
| | is hydrogen | | | CH, Y³ is N |
| P.2727 | X¹ is chloro, X² is C—Cl, X³ | trifluoromethyl | cyano | Y¹ is CH, Y² is |
| | is hydrogen | | | CH, Y³ is N |
| P.2728 | X¹ is chloro, X² is C—Cl, X³ | trifluoromethyl | cyclopropyl | Y¹ is CH, Y² is |
| | is hydrogen | | | CH, Y³ is N |
| P.2729 | X¹ is chloro, X² is C—Cl, X³ | trifluoromethyl | ethyl | Y¹ is CH, Y² is |
| | is hydrogen | | | CH, Y³ is N |
| P.2730 | X¹ is chloro, X² is C—Cl, X³ | trifluoromethyl | fluoro | Y¹ is CH, Y² is |
| | is hydrogen | | | CH, Y³ is N |
| P.2731 | X¹ is chloro, X² is C—Cl, X³ | trifluoromethyl | hydrogen | Y¹ is CH, Y² is |
| | is hydrogen | | | CH, Y³ is N |
| P.2732 | X¹ is chloro, X² is C—Cl, X³ | trifluoromethyl | methoxy | Y¹ is CH, Y² is |
| | is hydrogen | | | CH, Y³ is N |
| P.2733 | X¹ is chloro, X² is C—Cl, X³ | trifluoromethyl | methyl | Y¹ is CH, Y² is |
| | is hydrogen | | | CH, Y³ is N |
| P.2734 | X¹ is chloro, X² is C—Cl, X³ | trifluoromethyl | nitro | Y¹ is CH, Y² is |
| | is hydrogen | | | CH, Y³ is N |
| P.2735 | X¹ is chloro, X² is C—Cl, X³ | trifluoromethyl | trifluoromethoxy | Y¹ is CH, Y² is |
| | is hydrogen | | | CH, Y³ is N |
| P.2736 | X¹ is chloro, X² is C—Cl, X³ | trifluoromethyl | trifluoromethyl | Y¹ is CH, Y² is |
| | is hydrogen | | | CH, Y³ is N |
| P.2737 | X¹ is chloro, X² is C—Br, X³ | chlorodifluoromethyl | bromo | Y¹ is CH, Y² is |
| | is chloro | | | CH, Y³ is N |
| P.2738 | X¹ is chloro, X² is C—Br, X³ | chlorodifluoromethyl | chloro | Y¹ is CH, Y² is |
| | is chloro | | | CH, Y³ is N |
| P.2739 | X¹ is chloro, X² is C—Br, X³ | chlorodifluoromethyl | cyano | Y¹ is CH, Y² is |
| | is chloro | | | CH, Y³ is N |
| P.2740 | X¹ is chloro, X² is C—Br, X³ | chlorodifluoromethyl | cyclopropyl | Y¹ is CH, Y² is |
| | is chloro | | | CH, Y³ is N |
| P.2741 | X¹ is chloro, X² is C—Br, X³ | chlorodifluoromethyl | ethyl | Y¹ is CH, Y² is |
| | is chloro | | | CH, Y³ is N |
| P.2742 | X¹ is chloro, X² is C—Br, X³ | chlorodifluoromethyl | fluoro | Y¹ is CH, Y² is |
| | is chloro | | | CH, Y³ is N |
| P.2743 | X¹ is chloro, X² is C—Br, X³ | chlorodifluoromethyl | hydrogen | Y¹ is CH, Y² is |
| | is chloro | | | CH, Y³ is N |
| P.2744 | X¹ is chloro, X² is C—Br, X³ | chlorodifluoromethyl | methoxy | Y¹ is CH, Y² is |
| | is chloro | | | CH, Y³ is N |
| P.2745 | X¹ is chloro, X² is C—Br, X³ | chlorodifluoromethyl | methyl | Y¹ is CH, Y² is |
| | is chloro | | | CH, Y³ is N |
| P.2746 | X¹ is chloro, X² is C—Br, X³ | chlorodifluoromethyl | nitro | Y¹ is CH, Y² is |
| | is chloro | | | CH, Y³ is N |
| P.2747 | X¹ is chloro, X² is C—Br, X³ | chlorodifluoromethyl | trifluoromethoxy | Y¹ is CH, Y² is |
| | is chloro | | | CH, Y³ is N |
| P.2748 | X¹ is chloro, X² is C—Br, X³ | chlorodifluoromethyl | trifluoromethyl | Y¹ is CH, Y² is |
| | is chloro | | | CH, Y³ is N |
| P.2749 | X¹ is chloro, X² is C—Br, X³ | difluoromethyl | bromo | Y¹ is CH, Y² is |
| | is chloro | | | CH, Y³ is N |
| P.2750 | X¹ is chloro, X² is C—Br, X³ | difluoromethyl | chloro | Y¹ is CH, Y² is |
| | is chloro | | | CH, Y³ is N |
| P.2751 | X¹ is chloro, X² is C—Br, X³ | difluoromethyl | cyano | Y¹ is CH, Y² is |
| | is chloro | | | CH, Y³ is N |
| P.2752 | X¹ is chloro, X² is C—Br, X³ | difluoromethyl | cyclopropyl | Y¹ is CH, Y² is |
| | is chloro | | | CH, Y³ is N |
| P.2753 | X¹ is chloro, X² is C—Br, X³ | difluoromethyl | ethyl | Y¹ is CH, Y² is |
| | is chloro | | | CH, Y³ is N |
| P.2754 | X¹ is chloro, X² is C—Br, X³ | difluoromethyl | fluoro | Y¹ is CH, Y² is |
| | is chloro | | | CH, Y³ is N |
| P.2755 | X¹ is chloro, X² is C—Br, X³ | difluoromethyl | hydrogen | Y¹ is CH, Y² is |
| | is chloro | | | CH, Y³ is N |
| P.2756 | X¹ is chloro, X² is C—Br, X³ | difluoromethyl | methoxy | Y¹ is CH, Y² is |
| | is chloro | | | CH, Y³ is N |
| P.2757 | X¹ is chloro, X² is C—Br, X³ | difluoromethyl | methyl | Y¹ is CH, Y² is |
| | is chloro | | | CH, Y³ is N |
| P.2758 | X¹ is chloro, X² is C—Br, X³ | difluoromethyl | nitro | Y¹ is CH, Y² is |
| | is chloro | | | CH, Y³ is N |
| P.2759 | X¹ is chloro, X² is C—Br, X³ | difluoromethyl | trifluoromethoxy | Y¹ is CH, Y² is |
| | is chloro | | | CH, Y³ is N |
| P.2760 | X¹ is chloro, X² is C—Br, X³ | difluoromethyl | trifluoromethyl | Y¹ is CH, Y² is |
| | is chloro | | | CH, Y³ is N |
| P.2761 | X¹ is chloro, X² is C—Br, X³ | trifluoromethyl | bromo | Y¹ is CH, Y² is |
| | is chloro | | | CH, Y³ is N |
| P.2762 | X¹ is chloro, X² is C—Br, X³ | trifluoromethyl | chloro | Y¹ is CH, Y² is |
| | is chloro | | | CH, Y³ is N |
| P.2763 | X¹ is chloro, X² is C—Br, X³ | trifluoromethyl | cyano | Y¹ is CH, Y² is |
| | is chloro | | | CH, Y³ is N |
| P.2764 | X¹ is chloro, X² is C—Br, X³ | trifluoromethyl | cyclopropyl | Y¹ is CH, Y² is |
| | is chloro | | | CH, Y³ is N |
| P.2765 | X¹ is chloro, X² is C—Br, X³ | trifluoromethyl | ethyl | Y¹ is CH, Y² is |
| | is chloro | | | CH, Y³ is N |
| P.2766 | X¹ is chloro, X² is C—Br, X³ | trifluoromethyl | fluoro | Y¹ is CH, Y² is |
| | is chloro | | | CH, Y³ is N |
| P.2767 | X¹ is chloro, X² is C—Br, X³ | trifluoromethyl | hydrogen | Y¹ is CH, Y² is |
| | is chloro | | | CH, Y³ is N |
| P.2768 | X¹ is chloro, X² is C—Br, X³ | trifluoromethyl | methoxy | Y¹ is CH, Y² is |
| | is chloro | | | CH, Y³ is N |
| P.2769 | X¹ is chloro, X² is C—Br, X³ | trifluoromethyl | methyl | Y¹ is CH, Y² is |
| | is chloro | | | CH, Y³ is N |
| P.2770 | X¹ is chloro, X² is C—Br, X³ | trifluoromethyl | nitro | Y¹ is CH, Y² is |
| | is chloro | | | CH, Y³ is N |
| P.2771 | X¹ is chloro, X² is C—Br, X³ | trifluoromethyl | trifluoromethoxy | Y¹ is CH, Y² is |
| | is chloro | | | CH, Y³ is N |
| P.2772 | X¹ is chloro, X² is C—Br, X³ | trifluoromethyl | trifluoromethyl | Y¹ is CH, Y² is |
| | is chloro | | | CH, Y³ is N |
| P.2773 | X¹ is chloro, X² is C—F, X³ is | chlorodifluoromethyl | bromo | Y¹ is CH, Y² is |
| | chloro | | | CH, Y³ is N |
| P.2774 | X¹ is chloro, X² is C—F, X³ is | chlorodifluoromethyl | chloro | Y¹ is CH, Y² is |
| | chloro | | | CH, Y³ is N |
| P.2775 | X¹ is chloro, X² is C—F, X³ is | chlorodifluoromethyl | cyano | Y¹ is CH, Y² is |
| | chloro | | | CH, Y³ is N |
| P.2776 | X¹ is chloro, X² is C—F, X³ is | chlorodifluoromethyl | cyclopropyl | Y¹ is CH, Y² is |
| | chloro | | | CH, Y³ is N |
| P.2777 | X¹ is chloro, X² is C—F, X³ is | chlorodifluoromethyl | ethyl | Y¹ is CH, Y² is |
| | chloro | | | CH, Y³ is N |
| P.2778 | X¹ is chloro, X² is C—F, X³ is | chlorodifluoromethyl | fluoro | Y¹ is CH, Y² is |
| | chloro | | | CH, Y³ is N |
| P.2779 | X¹ is chloro, X² is C—F, X³ is | chlorodifluoromethyl | hydrogen | Y¹ is CH, Y² is |
| | chloro | | | CH, Y³ is N |
| P.2780 | X¹ is chloro, X² is C—F, X³ is | chlorodifluoromethyl | methoxy | Y¹ is CH, Y² is |
| | chloro | | | CH, Y³ is N |
| P.2781 | X¹ is chloro, X² is C—F, X³ is | chlorodifluoromethyl | methyl | Y¹ is CH, Y² is |
| | chloro | | | CH, Y³ is N |
| P.2782 | X¹ is chloro, X² is C—F, X³ is | chlorodifluoromethyl | nitro | Y¹ is CH, Y² is |
| | chloro | | | CH, Y³ is N |
| P.2783 | X¹ is chloro, X² is C—F, X³ is | chlorodifluoromethyl | trifluoromethoxy | Y¹ is CH, Y² is |
| | chloro | | | CH, Y³ is N |
| P.2784 | X¹ is chloro, X² is C—F, X³ is | chlorodifluoromethyl | trifluoromethyl | Y¹ is CH, Y² is |
| | chloro | | | CH, Y³ is N |
| P.2785 | X¹ is chloro, X² is C—F, X³ is | difluoromethyl | bromo | Y¹ is CH, Y² is |
| | chloro | | | CH, Y³ is N |
| P.2786 | X¹ is chloro, X² is C—F, X³ is | difluoromethyl | chloro | Y¹ is CH, Y² is |
| | chloro | | | CH, Y³ is N |
| P.2787 | X¹ is chloro, X² is C—F, X³ is | difluoromethyl | cyano | Y¹ is CH, Y² is |
| | chloro | | | CH, Y³ is N |
| P.2788 | X¹ is chloro, X² is C—F, X³ is | difluoromethyl | cyclopropyl | Y¹ is CH, Y² is |
| | chloro | | | CH, Y³ is N |
| P.2789 | X¹ is chloro, X² is C—F, X³ is | difluoromethyl | ethyl | Y¹ is CH, Y² is |
| | chloro | | | CH, Y³ is N |
| P.2790 | X¹ is chloro, X² is C—F, X³ is | difluoromethyl | fluoro | Y¹ is CH, Y² is |
| | chloro | | | CH, Y³ is N |
| P.2791 | X¹ is chloro, X² is C—F, X³ is | difluoromethyl | hydrogen | Y¹ is CH, Y² is |
| | chloro | | | CH, Y³ is N |
| P.2792 | X¹ is chloro, X² is C—F, X³ is | difluoromethyl | methoxy | Y¹ is CH, Y² is |
| | chloro | | | CH, Y³ is N |
| P.2793 | X¹ is chloro, X² is C—F, X³ is | difluoromethyl | methyl | Y¹ is CH, Y² is |
| | chloro | | | CH, Y³ is N |
| P.2794 | X¹ is chloro, X² is C—F, X³ is | difluoromethyl | nitro | Y¹ is CH, Y² is |
| | chloro | | | CH, Y³ is N |
| P.2795 | X¹ is chloro, X² is C—F, X³ is | difluoromethyl | trifluoromethoxy | Y¹ is CH, Y² is |
| | chloro | | | CH, Y³ is N |
| P.2796 | X¹ is chloro, X² is C—F, X³ is | difluoromethyl | trifluoromethyl | Y¹ is CH, Y² is |
| | chloro | | | CH, Y³ is N |
| P.2797 | X¹ is chloro, X² is C—F, X³ is | trifluoromethyl | bromo | Y¹ is CH, Y² is |
| | chloro | | | CH, Y³ is N |
| P.2798 | X¹ is chloro, X² is C—F, X³ is | trifluoromethyl | chloro | Y¹ is CH, Y² is |
| | chloro | | | CH, Y³ is N |
| P.2799 | X¹ is chloro, X² is C—F, X³ is | trifluoromethyl | cyano | Y¹ is CH, Y² is |
| | chloro | | | CH, Y³ is N |
| P.2800 | X¹ is chloro, X² is C—F, X³ is | trifluoromethyl | cyclopropyl | Y¹ is CH, Y² is |
| | chloro | | | CH, Y³ is N |
| P.2801 | X¹ is chloro, X² is C—F, X³ is | trifluoromethyl | ethyl | Y¹ is CH, Y² is |
| | chloro | | | CH, Y³ is N |
| P.2802 | X¹ is chloro, X² is C—F, X³ is | trifluoromethyl | fluoro | Y¹ is CH, Y² is |
| | chloro | | | CH, Y³ is N |
| P.2803 | X¹ is chloro, X² is C—F, X³ is | trifluoromethyl | hydrogen | Y¹ is CH, Y² is |
| | chloro | | | CH, Y³ is N |
| P.2804 | X¹ is chloro, X² is C—F, X³ is | trifluoromethyl | methoxy | Y¹ is CH, Y² is |
| | chloro | | | CH, Y³ is N |
| P.2805 | X¹ is chloro, X² is C—F, X³ is | trifluoromethyl | methyl | Y¹ is CH, Y² is |
| | chloro | | | CH, Y³ is N |
| P.2806 | X¹ is chloro, X² is C—F, X³ is | trifluoromethyl | nitro | Y¹ is CH, Y² is |
| | chloro | | | CH, Y³ is N |
| P.2807 | X¹ is chloro, X² is C—F, X³ is | trifluoromethyl | trifluoromethoxy | Y¹ is CH, Y² is |
| | chloro | | | CH, Y³ is N |
| P.2808 | X¹ is chloro, X² is C—F, X³ is | trifluoromethyl | trifluoromethyl | Y¹ is CH, Y² is |
| | chloro | | | CH, Y³ is N |
| P.2809 | X¹ is chloro, X² is C—Cl, X³ | chlorodifluoromethyl | bromo | Y¹ is CH, Y² is |
| | is chloro | | | CH, Y³ is N |
| P.2810 | X¹ is chloro, X² is C—Cl, X³ | chlorodifluoromethyl | chloro | Y¹ is CH, Y² is |
| | is chloro | | | CH, Y³ is N |
| P.2811 | X¹ is chloro, X² is C—Cl, X³ | chlorodifluoromethyl | cyano | Y¹ is CH, Y² is |
| | is chloro | | | CH, Y³ is N |
| P.2812 | X¹ is chloro, X² is C—Cl, X³ | chlorodifluoromethyl | cyclopropyl | Y¹ is CH, Y² is |
| | is chloro | | | CH, Y³ is N |
| P.2813 | X¹ is chloro, X² is C—Cl, X³ | chlorodifluoromethyl | ethyl | Y¹ is CH, Y² is |
| | is chloro | | | CH, Y³ is N |
| P.2814 | X¹ is chloro, X² is C—Cl, X³ | chlorodifluoromethyl | fluoro | Y¹ is CH, Y² is |
| | is chloro | | | CH, Y³ is N |
| P.2815 | X¹ is chloro, X² is C—Cl, X³ | chlorodifluoromethyl | hydrogen | Y¹ is CH, Y² is |
| | is chloro | | | CH, Y³ is N |
| P.2816 | X¹ is chloro, X² is C—Cl, X³ | chlorodifluoromethyl | methoxy | Y¹ is CH, Y² is |
| | is chloro | | | CH, Y³ is N |
| P.2817 | X¹ is chloro, X² is C—Cl, X³ | chlorodifluoromethyl | methyl | Y¹ is CH, Y² is |
| | is chloro | | | CH, Y³ is N |
| P.2818 | X¹ is chloro, X² is C—Cl, X³ | chlorodifluoromethyl | nitro | Y¹ is CH, Y² is |
| | is chloro | | | CH, Y³ is N |
| P.2819 | X¹ is chloro, X² is C—Cl, X³ | chlorodifluoromethyl | trifluoromethoxy | Y¹ is CH, Y² is |
| | is chloro | | | CH, Y³ is N |
| P.2820 | X¹ is chloro, X² is C—Cl, X³ | chlorodifluoromethyl | trifluoromethyl | Y¹ is CH, Y² is |
| | is chloro | | | CH, Y³ is N |
| P.2821 | X¹ is chloro, X² is C—Cl, X³ | difluoromethyl | bromo | Y¹ is CH, Y² is |
| | is chloro | | | CH, Y³ is N |
| P.2822 | X¹ is chloro, X² is C—Cl, X³ | difluoromethyl | chloro | Y¹ is CH, Y² is |
| | is chloro | | | CH, Y³ is N |
| P.2823 | X¹ is chloro, X² is C—Cl, X³ | difluoromethyl | cyano | Y¹ is CH, Y² is |
| | is chloro | | | CH, Y³ is N |
| P.2824 | X¹ is chloro, X² is C—Cl, X³ | difluoromethyl | cyclopropyl | Y¹ is CH, Y² is |
| | is chloro | | | CH, Y³ is N |
| P.2825 | X¹ is chloro, X² is C—Cl, X³ | difluoromethyl | ethyl | Y¹ is CH, Y² is |
| | is chloro | | | CH, Y³ is N |
| P.2826 | X¹ is chloro, X² is C—Cl, X³ | difluoromethyl | fluoro | Y¹ is CH, Y² is |
| | is chloro | | | CH, Y³ is N |
| P.2827 | X¹ is chloro, X² is C—Cl, X³ | difluoromethyl | hydrogen | Y¹ is CH, Y² is |
| | is chloro | | | CH, Y³ is N |
| P.2828 | X¹ is chloro, X² is C—Cl, X³ | difluoromethyl | methoxy | Y¹ is CH, Y² is |
| | is chloro | | | CH, Y³ is N |
| P.2829 | X¹ is chloro, X² is C—Cl, X³ | difluoromethyl | methyl | Y¹ is CH, Y² is |
| | is chloro | | | CH, Y³ is N |
| P.2830 | X¹ is chloro, X² is C—Cl, X³ | difluoromethyl | nitro | Y¹ is CH, Y² is |
| | is chloro | | | CH, Y³ is N |
| P.2831 | X¹ is chloro, X² is C—Cl, X³ | difluoromethyl | trifluoromethoxy | Y¹ is CH, Y² is |
| | is chloro | | | CH, Y³ is N |
| P.2832 | X¹ is chloro, X² is C—Cl, X³ | difluoromethyl | trifluoromethyl | Y¹ is CH, Y² is |
| | is chloro | | | CH, Y³ is N |
| P.2833 | X¹ is chloro, X² is C—Cl, X³ | trifluoromethyl | bromo | Y¹ is CH, Y² is |
| | is chloro | | | CH, Y³ is N |
| P.2834 | X¹ is chloro, X² is C—Cl, X³ | trifluoromethyl | chloro | Y¹ is CH, Y² is |
| | is chloro | | | CH, Y³ is N |
| P.2835 | X¹ is chloro, X² is C—Cl, X³ | trifluoromethyl | cyano | Y¹ is CH, Y² is |
| | is chloro | | | CH, Y³ is N |
| P.2836 | X¹ is chloro, X² is C—Cl, X³ | trifluoromethyl | cyclopropyl | Y¹ is CH, Y² is |
| | is chloro | | | CH, Y³ is N |
| P.2837 | X¹ is chloro, X² is C—Cl, X³ | trifluoromethyl | ethyl | Y¹ is CH, Y² is |
| | is chloro | | | CH, Y³ is N |
| P.2838 | X¹ is chloro, X² is C—Cl, X³ | trifluoromethyl | fluoro | Y¹ is CH, Y² is |
| | is chloro | | | CH, Y³ is N |
| P.2839 | X¹ is chloro, X² is C—Cl, X³ | trifluoromethyl | hydrogen | Y¹ is CH, Y² is |
| | is chloro | | | CH, Y³ is N |
| P.2840 | X¹ is chloro, X² is C—Cl, X³ | trifluoromethyl | methoxy | Y¹ is CH, Y² is |
| | is chloro | | | CH, Y³ is N |
| P.2841 | X¹ is chloro, X² is C—Cl, X³ | trifluoromethyl | methyl | Y¹ is CH, Y² is |
| | is chloro | | | CH, Y³ is N |
| P.2842 | X¹ is chloro, X² is C—Cl, X³ | trifluoromethyl | nitro | Y¹ is CH, Y² is |
| | is chloro | | | CH, Y³ is N |
| P.2843 | X¹ is chloro, X² is C—Cl, X³ | trifluoromethyl | trifluoromethoxy | Y¹ is CH, Y² is |
| | is chloro | | | CH, Y³ is N |
| P.2844 | X¹ is chloro, X² is C—Cl, X³ | trifluoromethyl | trifluoromethyl | Y¹ is CH, Y² is |
| | is chloro | | | CH, Y³ is N |
| P.2845 | X¹ is chloro, X² is C—I, X³ is | chlorodifluoromethyl | bromo | Y¹ is CH, Y² is |
| | chloro | | | CH, Y³ is N |
| P.2846 | X¹ is chloro, X² is C—I, X³ is | chlorodifluoromethyl | chloro | Y¹ is CH, Y² is |
| | chloro | | | CH, Y³ is N |
| P.2847 | X¹ is chloro, X² is C—I, X³ is | chlorodifluoromethyl | cyano | Y¹ is CH, Y² is |
| | chloro | | | CH, Y³ is N |
| P.2848 | X¹ is chloro, X² is C—I, X³ is | chlorodifluoromethyl | cyclopropyl | Y¹ is CH, Y² is |
| | chloro | | | CH, Y³ is N |
| P.2849 | X¹ is chloro, X² is C—I, X³ is | chlorodifluoromethyl | ethyl | Y¹ is CH, Y² is |
| | chloro | | | CH, Y³ is N |
| P.2850 | X¹ is chloro, X² is C—I, X³ is | chlorodifluoromethyl | fluoro | Y¹ is CH, Y² is |
| | chloro | | | CH, Y³ is N |
| P.2851 | X¹ is chloro, X² is C—I, X³ is | chlorodifluoromethyl | hydrogen | Y¹ is CH, Y² is |
| | chloro | | | CH, Y³ is N |
| P.2852 | X¹ is chloro, X² is C—I, X³ is | chlorodifluoromethyl | methoxy | Y¹ is CH, Y² is |
| | chloro | | | CH, Y³ is N |
| P.2853 | X¹ is chloro, X² is C—I, X³ is | chlorodifluoromethyl | methyl | Y¹ is CH, Y² is |
| | chloro | | | CH, Y³ is N |
| P.2854 | X¹ is chloro, X² is C—I, X³ is | chlorodifluoromethyl | nitro | Y¹ is CH, Y² is |
| | chloro | | | CH, Y³ is N |
| P.2855 | X¹ is chloro, X² is C—I, X³ is | chlorodifluoromethyl | trifluoromethoxy | Y¹ is CH, Y² is |
| | chloro | | | CH, Y³ is N |
| P.2856 | X¹ is chloro, X² is C—I, X³ is | chlorodifluoromethyl | trifluoromethyl | Y¹ is CH, Y² is |
| | chloro | | | CH, Y³ is N |
| P.2857 | X¹ is chloro, X² is C—I, X³ is | difluoromethyl | bromo | Y¹ is CH, Y² is |
| | chloro | | | CH, Y³ is N |
| P.2858 | X¹ is chloro, X² is C—I, X³ is | difluoromethyl | chloro | Y¹ is CH, Y² is |
| | chloro | | | CH, Y³ is N |
| P.2859 | X¹ is chloro, X² is C—I, X³ is | difluoromethyl | cyano | Y¹ is CH, Y² is |
| | chloro | | | CH, Y³ is N |
| P.2860 | X¹ is chloro, X² is C—I, X³ is | difluoromethyl | cyclopropyl | Y¹ is CH, Y² is |
| | chloro | | | CH, Y³ is N |
| P.2861 | X¹ is chloro, X² is C—I, X³ is | difluoromethyl | ethyl | Y¹ is CH, Y² is |
| | chloro | | | CH, Y³ is N |
| P.2862 | X¹ is chloro, X² is C—I, X³ is | difluoromethyl | fluoro | Y¹ is CH, Y² is |
| | chloro | | | CH, Y³ is N |
| P.2863 | X¹ is chloro, X² is C—I, X³ is | difluoromethyl | hydrogen | Y¹ is CH, Y² is |
| | chloro | | | CH, Y³ is N |
| P.2864 | X¹ is chloro, X² is C—I, X³ is | difluoromethyl | methoxy | Y¹ is CH, Y² is |
| | chloro | | | CH, Y³ is N |
| P.2865 | X¹ is chloro, X² is C—I, X³ is | difluoromethyl | methyl | Y¹ is CH, Y² is |
| | chloro | | | CH, Y³ is N |
| P.2866 | X¹ is chloro, X² is C—I, X³ is | difluoromethyl | nitro | Y¹ is CH, Y² is |
| | chloro | | | CH, Y³ is N |
| P.2867 | X¹ is chloro, X² is C—I, X³ is | difluoromethyl | trifluoromethoxy | Y¹ is CH, Y² is |
| | chloro | | | CH, Y³ is N |
| P.2868 | X¹ is chloro, X² is C—I, X³ is | difluoromethyl | trifluoromethyl | Y¹ is CH, Y² is |
| | chloro | | | CH, Y³ is N |
| P.2869 | X¹ is chloro, X² is C—I, X³ is | trifluoromethyl | bromo | Y¹ is CH, Y² is |
| | chloro | | | CH, Y³ is N |
| P.2870 | X¹ is chloro, X² is C—I, X³ is | trifluoromethyl | chloro | Y¹ is CH, Y² is |
| | chloro | | | CH, Y³ is N |
| P.2871 | X¹ is chloro, X² is C—I, X³ is | trifluoromethyl | cyano | Y¹ is CH, Y² is |
| | chloro | | | CH, Y³ is N |
| P.2872 | X¹ is chloro, X² is C—I, X³ is | trifluoromethyl | cyclopropyl | Y¹ is CH, Y² is |
| | chloro | | | CH, Y³ is N |
| P.2873 | X¹ is chloro, X² is C—I, X³ is | trifluoromethyl | ethyl | Y¹ is CH, Y² is |
| | chloro | | | CH, Y³ is N |
| P.2874 | X¹ is chloro, X² is C—I, X³ is | trifluoromethyl | fluoro | Y¹ is CH, Y² is |
| | chloro | | | CH, Y³ is N |
| P.2875 | X¹ is chloro, X² is C—I, X³ is | trifluoromethyl | hydrogen | Y¹ is CH, Y² is |
| | chloro | | | CH, Y³ is N |
| P.2876 | X¹ is chloro, X² is C—I, X³ is | trifluoromethyl | methoxy | Y¹ is CH, Y² is |
| | chloro | | | CH, Y³ is N |
| P.2877 | X¹ is chloro, X² is C—I, X³ is | trifluoromethyl | methyl | Y¹ is CH, Y² is |
| | chloro | | | CH, Y³ is N |
| P.2878 | X¹ is chloro, X² is C—I, X³ is | trifluoromethyl | nitro | Y¹ is CH, Y² is |
| | chloro | | | CH, Y³ is N |
| P.2879 | X¹ is chloro, X² is C—I, X³ is | trifluoromethyl | trifluoromethoxy | Y¹ is CH, Y² is |
| | chloro | | | CH, Y³ is N |
| P.2880 | X¹ is chloro, X² is C—I, X³ is | trifluoromethyl | trifluoromethyl | Y¹ is CH, Y² is |
| | chloro | | | CH, Y³ is N |
| P.2881 | X¹ is fluoro, X² is C—F, X³ is | chlorodifluoromethyl | bromo | Y¹ is CH, Y² is |
| | fluoro | | | CH, Y³ is N |
| P.2882 | X¹ is fluoro, X² is C—F, X³ is | chlorodifluoromethyl | chloro | Y¹ is CH, Y² is |
| | fluoro | | | CH, Y³ is N |
| P.2883 | X¹ is fluoro, X² is C—F, X³ is | chlorodifluoromethyl | cyano | Y¹ is CH, Y² is |
| | fluoro | | | CH, Y³ is N |
| P.2884 | X¹ is fluoro, X² is C—F, X³ is | chlorodifluoromethyl | cyclopropyl | Y¹ is CH, Y² is |
| | fluoro | | | CH, Y³ is N |
| P.2885 | X¹ is fluoro, X² is C—F, X³ is | chlorodifluoromethyl | ethyl | Y¹ is CH, Y² is |
| | fluoro | | | CH, Y³ is N |
| P.2886 | X¹ is fluoro, X² is C—F, X³ is | chlorodifluoromethyl | fluoro | Y¹ is CH, Y² is |
| | fluoro | | | CH, Y³ is N |
| P.2887 | X¹ is fluoro, X² is C—F, X³ is | chlorodifluoromethyl | hydrogen | Y¹ is CH, Y² is |
| | fluoro | | | CH, Y³ is N |
| P.2888 | X¹ is fluoro, X² is C—F, X³ is | chlorodifluoromethyl | methoxy | Y¹ is CH, Y² is |
| | fluoro | | | CH, Y³ is N |
| P.2889 | X¹ is fluoro, X² is C—F, X³ is | chlorodifluoromethyl | methyl | Y¹ is CH, Y² is |
| | fluoro | | | CH, Y³ is N |
| P.2890 | X¹ is fluoro, X² is C—F, X³ is | chlorodifluoromethyl | nitro | Y¹ is CH, Y² is |
| | fluoro | | | CH, Y³ is N |
| P.2891 | X¹ is fluoro, X² is C—F, X³ is | chlorodifluoromethyl | trifluoromethoxy | Y¹ is CH, Y² is |
| | fluoro | | | CH, Y³ is N |
| P.2892 | X¹ is fluoro, X² is C—F, X³ is | chlorodifluoromethyl | trifluoromethyl | Y¹ is CH, Y² is |
| | fluoro | | | CH, Y³ is N |
| P.2893 | X¹ is fluoro, X² is C—F, X³ is | difluoromethyl | bromo | Y¹ is CH, Y² is |
| | fluoro | | | CH, Y³ is N |
| P.2894 | X¹ is fluoro, X² is C—F, X³ is | difluoromethyl | chloro | Y¹ is CH, Y² is |
| | fluoro | | | CH, Y³ is N |
| P.2895 | X¹ is fluoro, X² is C—F, X³ is | difluoromethyl | cyano | Y¹ is CH, Y² is |
| | fluoro | | | CH, Y³ is N |
| P.2896 | X¹ is fluoro, X² is C—F, X³ is | difluoromethyl | cyclopropyl | Y¹ is CH, Y² is |
| | fluoro | | | CH, Y³ is N |
| P.2897 | X¹ is fluoro, X² is C—F, X³ is | difluoromethyl | ethyl | Y¹ is CH, Y² is |
| | fluoro | | | CH, Y³ is N |
| P.2898 | X¹ is fluoro, X² is C—F, X³ is | difluoromethyl | fluoro | Y¹ is CH, Y² is |
| | fluoro | | | CH, Y³ is N |
| P.2899 | X¹ is fluoro, X² is C—F, X³ is | difluoromethyl | hydrogen | Y¹ is CH, Y² is |
| | fluoro | | | CH, Y³ is N |
| P.2900 | X¹ is fluoro, X² is C—F, X³ is | difluoromethyl | methoxy | Y¹ is CH, Y² is |
| | fluoro | | | CH, Y³ is N |
| P.2901 | X¹ is fluoro, X² is C—F, X³ is | difluoromethyl | methyl | Y¹ is CH, Y² is |
| | fluoro | | | CH, Y³ is N |
| P.2902 | X¹ is fluoro, X² is C—F, X³ is | difluoromethyl | nitro | Y¹ is CH, Y² is |
| | fluoro | | | CH, Y³ is N |
| P.2903 | X¹ is fluoro, X² is C—F, X³ is | difluoromethyl | trifluoromethoxy | Y¹ is CH, Y² is |
| | fluoro | | | CH, Y³ is N |
| P.2904 | X¹ is fluoro, X² is C—F, X³ is | difluoromethyl | trifluoromethyl | Y¹ is CH, Y² is |
| | fluoro | | | CH, Y³ is N |
| P.2905 | X¹ is fluoro, X² is C—F, X³ is | trifluoromethyl | bromo | Y¹ is CH, Y² is |
| | fluoro | | | CH, Y³ is N |
| P.2906 | X¹ is fluoro, X² is C—F, X³ is | trifluoromethyl | chloro | Y¹ is CH, Y² is |
| | fluoro | | | CH, Y³ is N |
| P.2907 | X¹ is fluoro, X² is C—F, X³ is | trifluoromethyl | cyano | Y¹ is CH, Y² is |
| | fluoro | | | CH, Y³ is N |
| P.2908 | X¹ is fluoro, X² is C—F, X³ is | trifluoromethyl | cyclopropyl | Y¹ is CH, Y² is |
| | fluoro | | | CH, Y³ is N |
| P.2909 | X¹ is fluoro, X² is C—F, X³ is | trifluoromethyl | ethyl | Y¹ is CH, Y² is |
| | fluoro | | | CH, Y³ is N |
| P.2910 | X¹ is fluoro, X² is C—F, X³ is | trifluoromethyl | fluoro | Y¹ is CH, Y² is |
| | fluoro | | | CH, Y³ is N |
| P.2911 | X¹ is fluoro, X² is C—F, X³ is | trifluoromethyl | hydrogen | Y¹ is CH, Y² is |
| | fluoro | | | CH, Y³ is N |
| P.2912 | X¹ is fluoro, X² is C—F, X³ is | trifluoromethyl | methoxy | Y¹ is CH, Y² is |
| | fluoro | | | CH, Y³ is N |
| P.2913 | X¹ is fluoro, X² is C—F, X³ is | trifluoromethyl | methyl | Y¹ is CH, Y² is |
| | fluoro | | | CH, Y³ is N |
| P.2914 | X¹ is fluoro, X² is C—F, X³ is | trifluoromethyl | nitro | Y¹ is CH, Y² is |
| | fluoro | | | CH, Y³ is N |
| P.2915 | X¹ is fluoro, X² is C—F, X³ is | trifluoromethyl | trifluoromethoxy | Y¹ is CH, Y² is |
| | fluoro | | | CH, Y³ is N |
| P.2916 | X¹ is fluoro, X² is C—F, X³ is | trifluoromethyl | trifluoromethyl | Y¹ is CH, Y² is |
| | fluoro | | | CH, Y³ is N |
| P.2917 | X¹ is chloro, X² is CH, X³ is | chlorodifluoromethyl | bromo | Y¹ is CH, Y² is |
| | bromo | | | CH, Y³ is N |
| P.2918 | X¹ is chloro, X² is CH, X³ is | chlorodifluoromethyl | chloro | Y¹ is CH, Y² is |
| | bromo | | | CH, Y³ is N |
| P.2919 | X¹ is chloro, X² is CH, X³ is | chlorodifluoromethyl | cyano | Y¹ is CH, Y² is |
| | bromo | | | CH, Y³ is N |
| P.2920 | X¹ is chloro, X² is CH, X³ is | chlorodifluoromethyl | cyclopropyl | Y¹ is CH, Y² is |
| | bromo | | | CH, Y³ is N |
| P.2921 | X¹ is chloro, X² is CH, X³ is | chlorodifluoromethyl | ethyl | Y¹ is CH, Y² is |
| | bromo | | | CH, Y³ is N |
| P.2922 | X¹ is chloro, X² is CH, X³ is | chlorodifluoromethyl | fluoro | Y¹ is CH, Y² is |
| | bromo | | | CH, Y³ is N |
| P.2923 | X¹ is chloro, X² is CH, X³ is | chlorodifluoromethyl | hydrogen | Y¹ is CH, Y² is |
| | bromo | | | CH, Y³ is N |
| P.2924 | X¹ is chloro, X² is CH, X³ is | chlorodifluoromethyl | methoxy | Y¹ is CH, Y² is |
| | bromo | | | CH, Y³ is N |
| P.2925 | X¹ is chloro, X² is CH, X³ is | chlorodifluoromethyl | methyl | Y¹ is CH, Y² is |
| | bromo | | | CH, Y³ is N |
| P.2926 | X¹ is chloro, X² is CH, X³ is | chlorodifluoromethyl | nitro | Y¹ is CH, Y² is |
| | bromo | | | CH, Y³ is N |
| P.2927 | X¹ is chloro, X² is CH, X³ is | chlorodifluoromethyl | trifluoromethoxy | Y¹ is CH, Y² is |
| | bromo | | | CH, Y³ is N |
| P.2928 | X¹ is chloro, X² is CH, X³ is | chlorodifluoromethyl | trifluoromethyl | Y¹ is CH, Y² is |
| | bromo | | | CH, Y³ is N |
| P.2929 | X¹ is chloro, X² is CH, X³ is | difluoromethyl | bromo | Y¹ is CH, Y² is |
| | bromo | | | CH, Y³ is N |
| P.2930 | X¹ is chloro, X² is CH, X³ is | difluoromethyl | chloro | Y¹ is CH, Y² is |
| | bromo | | | CH, Y³ is N |
| P.2931 | X¹ is chloro, X² is CH, X³ is | difluoromethyl | cyano | Y¹ is CH, Y² is |
| | bromo | | | CH, Y³ is N |
| P.2932 | X¹ is chloro, X² is CH, X³ is | difluoromethyl | cyclopropyl | Y¹ is CH, Y² is |
| | bromo | | | CH, Y³ is N |
| P.2933 | X¹ is chloro, X² is CH, X³ is | difluoromethyl | ethyl | Y¹ is CH, Y² is |
| | bromo | | | CH, Y³ is N |
| P.2934 | X¹ is chloro, X² is CH, X³ is | difluoromethyl | fluoro | Y¹ is CH, Y² is |
| | bromo | | | CH, Y³ is N |
| P.2935 | X¹ is chloro, X² is CH, X³ is | difluoromethyl | hydrogen | Y¹ is CH, Y² is |
| | bromo | | | CH, Y³ is N |
| P.2936 | X¹ is chloro, X² is CH, X³ is | difluoromethyl | methoxy | Y¹ is CH, Y² is |
| | bromo | | | CH, Y³ is N |
| P.2937 | X¹ is chloro, X² is CH, X³ is | difluoromethyl | methyl | Y¹ is CH, Y² is |
| | bromo | | | CH, Y³ is N |
| P.2938 | X¹ is chloro, X² is CH, X³ is | difluoromethyl | nitro | Y¹ is CH, Y² is |
| | bromo | | | CH, Y³ is N |
| P.2939 | X¹ is chloro, X² is CH, X³ is | difluoromethyl | trifluoromethoxy | Y¹ is CH, Y² is |
| | bromo | | | CH, Y³ is N |
| P.2940 | X¹ is chloro, X² is CH, X³ is | difluoromethyl | trifluoromethyl | Y¹ is CH, Y² is |
| | bromo | | | CH, Y³ is N |
| P.2941 | X¹ is chloro, X² is CH, X³ is | trifluoromethyl | bromo | Y¹ is CH, Y² is |
| | bromo | | | CH, Y³ is N |
| P.2942 | X¹ is chloro, X² is CH, X³ is | trifluoromethyl | chloro | Y¹ is CH, Y² is |
| | bromo | | | CH, Y³ is N |
| P.2943 | X¹ is chloro, X² is CH, X³ is | trifluoromethyl | cyano | Y¹ is CH, Y² is |
| | bromo | | | CH, Y³ is N |
| P.2944 | X¹ is chloro, X² is CH, X³ is | trifluoromethyl | cyclopropyl | Y¹ is CH, Y² is |
| | bromo | | | CH, Y³ is N |
| P.2945 | X¹ is chloro, X² is CH, X³ is | trifluoromethyl | ethyl | Y¹ is CH, Y² is |
| | bromo | | | CH, Y³ is N |
| P.2946 | X¹ is chloro, X² is CH, X³ is | trifluoromethyl | fluoro | Y¹ is CH, Y² is |
| | bromo | | | CH, Y³ is N |
| P.2947 | X¹ is chloro, X² is CH, X³ is | trifluoromethyl | hydrogen | Y¹ is CH, Y² is |
| | bromo | | | CH, Y³ is N |
| P.2948 | X¹ is chloro, X² is CH, X³ is | trifluoromethyl | methoxy | Y¹ is CH, Y² is |
| | bromo | | | CH, Y³ is N |
| P.2949 | X¹ is chloro, X² is CH, X³ is | trifluoromethyl | methyl | Y¹ is CH, Y² is |
| | bromo | | | CH, Y³ is N |
| P.2950 | X¹ is chloro, X² is CH, X³ is | trifluoromethyl | nitro | Y¹ is CH, Y² is |
| | bromo | | | CH, Y³ is N |
| P.2951 | X¹ is chloro, X² is CH, X³ is | trifluoromethyl | trifluoromethoxy | Y¹ is CH, Y² is |
| | bromo | | | CH, Y³ is N |
| P.2952 | X¹ is chloro, X² is CH, X³ is | trifluoromethyl | trifluoromethyl | Y¹ is CH, Y² is |
| | bromo | | | CH, Y³ is N |
| P.2953 | X¹ is chloro, X² is CH, X³ is | chlorodifluoromethyl | bromo | Y¹ is CH, Y² is |
| | bromo | | | CH, Y³ is N |
| P.2954 | X¹ is chloro, X² is CH, X³ is | chlorodifluoromethyl | chloro | Y¹ is CH, Y² is |
| | bromo | | | CH, Y³ is N |
| P.2955 | X¹ is chloro, X² is CH, X³ is | chlorodifluoromethyl | cyano | Y¹ is CH, Y² is |
| | bromo | | | CH, Y³ is N |
| P.2956 | X¹ is chloro, X² is CH, X³ is | chlorodifluoromethyl | cyclopropyl | Y¹ is CH, Y² is |
| | bromo | | | CH, Y³ is N |
| P.2957 | X¹ is chloro, X² is CH, X³ is | chlorodifluoromethyl | ethyl | Y¹ is CH, Y² is |
| | bromo | | | CH, Y³ is N |
| P.2958 | X¹ is chloro, X² is CH, X³ is | chlorodifluoromethyl | fluoro | Y¹ is CH, Y² is |
| | bromo | | | CH, Y³ is N |
| P.2959 | X¹ is chloro, X² is CH, X³ is | chlorodifluoromethyl | hydrogen | Y¹ is CH, Y² is |
| | bromo | | | CH, Y³ is N |
| P.2960 | X¹ is chloro, X² is CH, X³ is | chlorodifluoromethyl | methoxy | Y¹ is CH, Y² is |
| | bromo | | | CH, Y³ is N |
| P.2961 | X¹ is chloro, X² is CH, X³ is | chlorodifluoromethyl | methyl | Y¹ is CH, Y² is |
| | bromo | | | CH, Y³ is N |
| P.2962 | X¹ is chloro, X² is CH, X³ is | chlorodifluoromethyl | nitro | Y¹ is CH, Y² is |
| | bromo | | | CH, Y³ is N |
| P.2963 | X¹ is chloro, X² is CH, X³ is | chlorodifluoromethyl | trifluoromethoxy | Y¹ is CH, Y² is |
| | bromo | | | CH, Y³ is N |
| P.2964 | X¹ is chloro, X² is CH, X³ is | chlorodifluoromethyl | trifluoromethyl | Y¹ is CH, Y² is |
| | bromo | | | CH, Y³ is N |
| P.2965 | X¹ is chloro, X² is CH, X³ is | difluoromethyl | bromo | Y¹ is CH, Y² is |
| | bromo | | | CH, Y³ is N |
| P.2966 | X¹ is chloro, X² is CH, X³ is | difluoromethyl | chloro | Y¹ is CH, Y² is |
| | bromo | | | CH, Y³ is N |
| P.2967 | X¹ is chloro, X² is CH, X³ is | difluoromethyl | cyano | Y¹ is CH, Y² is |
| | bromo | | | CH, Y³ is N |
| P.2968 | X¹ is chloro, X² is CH, X³ is | difluoromethyl | cyclopropyl | Y¹ is CH, Y² is |
| | bromo | | | CH, Y³ is N |
| P.2969 | X¹ is chloro, X² is CH, X³ is | difluoromethyl | ethyl | Y¹ is CH, Y² is |
| | bromo | | | CH, Y³ is N |
| P.2970 | X¹ is chloro, X² is CH, X³ is | difluoromethyl | fluoro | Y¹ is CH, Y² is |
| | bromo | | | CH, Y³ is N |
| P.2971 | X¹ is chloro, X² is CH, X³ is | difluoromethyl | hydrogen | Y¹ is CH, Y² is |
| | bromo | | | CH, Y³ is N |
| P.2972 | X¹ is chloro, X² is CH, X³ is | difluoromethyl | methoxy | Y¹ is CH, Y² is |
| | bromo | | | CH, Y³ is N |
| P.2973 | X¹ is chloro, X² is CH, X³ is | difluoromethyl | methyl | Y¹ is CH, Y² is |
| | bromo | | | CH, Y³ is N |
| P.2974 | X¹ is chloro, X² is CH, X³ is | difluoromethyl | nitro | Y¹ is CH, Y² is |
| | bromo | | | CH, Y³ is N |
| P.2975 | X¹ is chloro, X² is CH, X³ is | difluoromethyl | trifluoromethoxy | Y¹ is CH, Y² is |
| | bromo | | | CH, Y³ is N |
| P.2976 | X¹ is chloro, X² is CH, X³ is | difluoromethyl | trifluoromethyl | Y¹ is CH, Y² is |
| | bromo | | | CH, Y³ is N |
| P.2977 | X¹ is chloro, X² is CH, X³ is | trifluoromethyl | bromo | Y¹ is CH, Y² is |
| | bromo | | | CH, Y³ is N |
| P.2978 | X¹ is chloro, X² is CH, X³ is | trifluoromethyl | chloro | Y¹ is CH, Y² is |
| | bromo | | | CH, Y³ is N |
| P.2979 | X¹ is chloro, X² is CH, X³ is | trifluoromethyl | cyano | Y¹ is CH, Y² is |
| | bromo | | | CH, Y³ is N |
| P.2980 | X¹ is chloro, X² is CH, X³ is | trifluoromethyl | cyclopropyl | Y¹ is CH, Y² is |
| | bromo | | | CH, Y³ is N |
| P.2981 | X¹ is chloro, X² is CH, X³ is | trifluoromethyl | ethyl | Y¹ is CH, Y² is |
| | bromo | | | CH, Y³ is N |
| P.2982 | X¹ is chloro, X² is CH, X³ is | trifluoromethyl | fluoro | Y¹ is CH, Y² is |
| | bromo | | | CH, Y³ is N |
| P.2983 | X¹ is chloro, X² is CH, X³ is | trifluoromethyl | hydrogen | Y¹ is CH, Y² is |
| | bromo | | | CH, Y³ is N |
| P.2984 | X¹ is chloro, X² is CH, X³ is | trifluoromethyl | methoxy | Y¹ is CH, Y² is |
| | bromo | | | CH, Y³ is N |
| P.2985 | X¹ is chloro, X² is CH, X³ is | trifluoromethyl | methyl | Y¹ is CH, Y² is |
| | bromo | | | CH, Y³ is N |
| P.2986 | X¹ is chloro, X² is CH, X³ is | trifluoromethyl | nitro | Y¹ is CH, Y² is |
| | bromo | | | CH, Y³ is N |
| P.2987 | X¹ is chloro, X² is CH, X³ is | trifluoromethyl | trifluoromethoxy | Y¹ is CH, Y² is |
| | bromo | | | CH, Y³ is N |
| P.2988 | X¹ is chloro, X² is CH, X³ is | trifluoromethyl | trifluoromethyl | Y¹ is CH, Y² is |
| | bromo | | | CH, Y³ is N |
| P.2989 | X¹ is chloro, X² is CH, X³ is | chlorodifluoromethyl | bromo | Y¹ is CH, Y² is |
| | bromo | | | CH, Y³ is N |
| P.2990 | X¹ is chloro, X² is CH, X³ is | chlorodifluoromethyl | chloro | Y¹ is CH, Y² is |
| | bromo | | | CH, Y³ is N |
| P.2991 | X¹ is chloro, X² is CH, X³ is | chlorodifluoromethyl | cyano | Y¹ is CH, Y² is |
| | bromo | | | CH, Y³ is N |
| P.2992 | X¹ is chloro, X² is CH, X³ is | chlorodifluoromethyl | cyclopropyl | Y¹ is CH, Y² is |
| | bromo | | | CH, Y³ is N |
| P.2993 | X¹ is chloro, X² is CH, X³ is | chlorodifluoromethyl | ethyl | Y¹ is CH, Y² is |
| | bromo | | | CH, Y³ is N |
| P.2994 | X¹ is chloro, X² is CH, X³ is | chlorodifluoromethyl | fluoro | Y¹ is CH, Y² is |
| | bromo | | | CH, Y³ is N |
| P.2995 | X¹ is chloro, X² is CH, X³ is | chlorodifluoromethyl | hydrogen | Y¹ is CH, Y² is |
| | bromo | | | CH, Y³ is N |
| P.2996 | X¹ is chloro, X² is CH, X³ is | chlorodifluoromethyl | methoxy | Y¹ is CH, Y² is |
| | bromo | | | CH, Y³ is N |
| P.2997 | X¹ is chloro, X² is CH, X³ is | chlorodifluoromethyl | methyl | Y¹ is CH, Y² is |
| | bromo | | | CH, Y³ is N |
| P.2998 | X¹ is chloro, X² is CH, X³ is | chlorodifluoromethyl | nitro | Y¹ is CH, Y² is |
| | bromo | | | CH, Y³ is N |
| P.2999 | X¹ is chloro, X² is CH, X³ is | chlorodifluoromethyl | trifluoromethoxy | Y¹ is CH, Y² is |
| | bromo | | | CH, Y³ is N |
| P.3000 | X¹ is chloro, X² is CH, X³ is | chlorodifluoromethyl | trifluoromethyl | Y¹ is CH, Y² is |
| | bromo | | | CH, Y³ is N |
| P.3001 | X¹ is chloro, X² is CH, X³ is | difluoromethyl | bromo | Y¹ is CH, Y² is |
| | bromo | | | CH, Y³ is N |
| P.3002 | X¹ is chloro, X² is CH, X³ is | difluoromethyl | chloro | Y¹ is CH, Y² is |
| | bromo | | | CH, Y³ is N |
| P.3003 | X¹ is chloro, X² is CH, X³ is | difluoromethyl | cyano | Y¹ is CH, Y² is |
| | bromo | | | CH, Y³ is N |
| P.3004 | X¹ is chloro, X² is CH, X³ is | difluoromethyl | cyclopropyl | Y¹ is CH, Y² is |
| | bromo | | | CH, Y³ is N |
| P.3005 | X¹ is chloro, X² is CH, X³ is | difluoromethyl | ethyl | Y¹ is CH, Y² is |
| | bromo | | | CH, Y³ is N |
| P.3006 | X¹ is chloro, X² is CH, X³ is | difluoromethyl | fluoro | Y¹ is CH, Y² is |
| | bromo | | | CH, Y³ is N |
| P.3007 | X¹ is chloro, X² is CH, X³ is | difluoromethyl | hydrogen | Y¹ is CH, Y² is |
| | bromo | | | CH, Y³ is N |
| P.3008 | X¹ is chloro, X² is CH, X³ is | difluoromethyl | methoxy | Y¹ is CH, Y² is |
| | bromo | | | CH, Y³ is N |
| P.3009 | X¹ is chloro, X² is CH, X³ is | difluoromethyl | methyl | Y¹ is CH, Y² is |
| | bromo | | | CH, Y³ is N |
| P.3010 | X¹ is chloro, X² is CH, X³ is | difluoromethyl | nitro | Y¹ is CH, Y² is |
| | bromo | | | CH, Y³ is N |
| P.3011 | X¹ is chloro, X² is CH, X³ is | difluoromethyl | trifluoromethoxy | Y¹ is CH, Y² is |
| | bromo | | | CH, Y³ is N |
| P.3012 | X¹ is chloro, X² is CH, X³ is | difluoromethyl | trifluoromethyl | Y¹ is CH, Y² is |
| | bromo | | | CH, Y³ is N |
| P.3013 | X¹ is chloro, X² is CH, X³ is | trifluoromethyl | bromo | Y¹ is CH, Y² is |
| | bromo | | | CH, Y³ is N |
| P.3014 | X¹ is chloro, X² is CH, X³ is | trifluoromethyl | chloro | Y¹ is CH, Y² is |
| | bromo | | | CH, Y³ is N |
| P.3015 | X¹ is chloro, X² is CH, X³ is | trifluoromethyl | cyano | Y¹ is CH, Y² is |
| | bromo | | | CH, Y³ is N |
| P.3016 | X¹ is chloro, X² is CH, X³ is | trifluoromethyl | cyclopropyl | Y¹ is CH, Y² is |
| | bromo | | | CH, Y³ is N |
| P.3017 | X¹ is chloro, X² is CH, X³ is | trifluoromethyl | ethyl | Y¹ is CH, Y² is |
| | bromo | | | CH, Y³ is N |
| P.3018 | X¹ is chloro, X² is CH, X³ is | trifluoromethyl | fluoro | Y¹ is CH, Y² is |
| | bromo | | | CH, Y³ is N |
| P.3019 | X¹ is chloro, X² is CH, X³ is | trifluoromethyl | hydrogen | Y¹ is CH, Y² is |
| | bromo | | | CH, Y³ is N |
| P.3020 | X¹ is chloro, X² is CH, X³ is | trifluoromethyl | methoxy | Y¹ is CH, Y² is |
| | bromo | | | CH, Y³ is N |
| P.3021 | X¹ is chloro, X² is CH, X³ is | trifluoromethyl | methyl | Y¹ is CH, Y² is |
| | bromo | | | CH, Y³ is N |
| P.3022 | X¹ is chloro, X² is CH, X³ is | trifluoromethyl | nitro | Y¹ is CH, Y² is |
| | bromo | | | CH, Y³ is N |
| P.3023 | X¹ is chloro, X² is CH, X³ is | trifluoromethyl | trifluoromethoxy | Y¹ is CH, Y² is |
| | bromo | | | CH, Y³ is N |
| P.3024 | X¹ is chloro, X² is CH, X³ is | trifluoromethyl | trifluoromethyl | Y¹ is CH, Y² is |
| | bromo | | | CH, Y³ is N |
| P.3025 | X¹ is chloro, X² is C—Cl, X³ | chlorodifluoromethyl | bromo | Y¹ is CH, Y² is |
| | is trifluoromethyl | | | CH, Y³ is N |
| P.3026 | X¹ is chloro, X² is C—Cl, X³ | chlorodifluoromethyl | chloro | Y¹ is CH, Y² is |
| | is trifluoromethyl | | | CH, Y³ is N |
| P.3027 | X¹ is chloro, X² is C—Cl, X³ | chlorodifluoromethyl | cyano | Y¹ is CH, Y² is |
| | is trifluoromethyl | | | CH, Y³ is N |
| P.3028 | X¹ is chloro, X² is C—Cl, X³ | chlorodifluoromethyl | cyclopropyl | Y¹ is CH, Y² is |
| | is trifluoromethyl | | | CH, Y³ is N |
| P.3029 | X¹ is chloro, X² is C—Cl, X³ | chlorodifluoromethyl | ethyl | Y¹ is CH, Y² is |
| | is trifluoromethyl | | | CH, Y³ is N |
| P.3030 | X¹ is chloro, X² is C—Cl, X³ | chlorodifluoromethyl | fluoro | Y¹ is CH, Y² is |
| | is trifluoromethyl | | | CH, Y³ is N |
| P.3031 | X¹ is chloro, X² is C—Cl, X³ | chlorodifluoromethyl | hydrogen | Y¹ is CH, Y² is |
| | is trifluoromethyl | | | CH, Y³ is N |
| P.3032 | X¹ is chloro, X² is C—Cl, X³ | chlorodifluoromethyl | methoxy | Y¹ is CH, Y² is |
| | is trifluoromethyl | | | CH, Y³ is N |
| P.3033 | X¹ is chloro, X² is C—Cl, X³ | chlorodifluoromethyl | methyl | Y¹ is CH, Y² is |
| | is trifluoromethyl | | | CH, Y³ is N |
| P.3034 | X¹ is chloro, X² is C—Cl, X³ | chlorodifluoromethyl | nitro | Y¹ is CH, Y² is |
| | is trifluoromethyl | | | CH, Y³ is N |
| P.3035 | X¹ is chloro, X² is C—Cl, X³ | chlorodifluoromethyl | trifluoromethoxy | Y¹ is CH, Y² is |
| | is trifluoromethyl | | | CH, Y³ is N |
| P.3036 | X¹ is chloro, X² is C—Cl, X³ | chlorodifluoromethyl | trifluoromethyl | Y¹ is CH, Y² is |
| | is trifluoromethyl | | | CH, Y³ is N |
| P.3037 | X¹ is chloro, X² is C—Cl, X³ | difluoromethyl | bromo | Y¹ is CH, Y² is |
| | is trifluoromethyl | | | CH, Y³ is N |
| P.3038 | X¹ is chloro, X² is C—Cl, X³ | difluoromethyl | chloro | Y¹ is CH, Y² is |
| | is trifluoromethyl | | | CH, Y³ is N |
| P.3039 | X¹ is chloro, X² is C—Cl, X³ | difluoromethyl | cyano | Y¹ is CH, Y² is |
| | is trifluoromethyl | | | CH, Y³ is N |
| P.3040 | X¹ is chloro, X² is C—Cl, X³ | difluoromethyl | cyclopropyl | Y¹ is CH, Y² is |
| | is trifluoromethyl | | | CH, Y³ is N |
| P.3041 | X¹ is chloro, X² is C—Cl, X³ | difluoromethyl | ethyl | Y¹ is CH, Y² is |
| | is trifluoromethyl | | | CH, Y³ is N |
| P.3042 | X¹ is chloro, X² is C—Cl, X³ | difluoromethyl | fluoro | Y¹ is CH, Y² is |
| | is trifluoromethyl | | | CH, Y³ is N |
| P.3043 | X¹ is chloro, X² is C—Cl, X³ | difluoromethyl | hydrogen | Y¹ is CH, Y² is |
| | is trifluoromethyl | | | CH, Y³ is N |
| P.3044 | X¹ is chloro, X² is C—Cl, X³ | difluoromethyl | methoxy | Y¹ is CH, Y² is |
| | is trifluoromethyl | | | CH, Y³ is N |
| P.3045 | X¹ is chloro, X² is C—Cl, X³ | difluoromethyl | methyl | Y¹ is CH, Y² is |
| | is trifluoromethyl | | | CH, Y³ is N |
| P.3046 | X¹ is chloro, X² is C—Cl, X³ | difluoromethyl | nitro | Y¹ is CH, Y² is |
| | is trifluoromethyl | | | CH, Y³ is N |
| P.3047 | X¹ is chloro, X² is C—Cl, X³ | difluoromethyl | trifluoromethoxy | Y¹ is CH, Y² is |
| | is trifluoromethyl | | | CH, Y³ is N |
| P.3048 | X¹ is chloro, X² is C—Cl, X³ | difluoromethyl | trifluoromethyl | Y¹ is CH, Y² is |
| | is trifluoromethyl | | | CH, Y³ is N |
| P.3049 | X¹ is chloro, X² is C—Cl, X³ | trifluoromethyl | bromo | Y¹ is CH, Y² is |
| | is trifluoromethyl | | | CH, Y³ is N |
| P.3050 | X¹ is chloro, X² is C—Cl, X³ | trifluoromethyl | chloro | Y¹ is CH, Y² is |
| | is trifluoromethyl | | | CH, Y³ is N |
| P.3051 | X¹ is chloro, X² is C—Cl, X³ | trifluoromethyl | cyano | Y¹ is CH, Y² is |
| | is trifluoromethyl | | | CH, Y³ is N |
| P.3052 | X¹ is chloro, X² is C—Cl, X³ | trifluoromethyl | cyclopropyl | Y¹ is CH, Y² is |
| | is trifluoromethyl | | | CH, Y³ is N |
| P.3053 | X¹ is chloro, X² is C—Cl, X³ | trifluoromethyl | ethyl | Y¹ is CH, Y² is |
| | is trifluoromethyl | | | CH, Y³ is N |
| P.3054 | X¹ is chloro, X² is C—Cl, X³ | trifluoromethyl | fluoro | Y¹ is CH, Y² is |
| | is trifluoromethyl | | | CH, Y³ is N |
| P.3055 | X¹ is chloro, X² is C—Cl, X³ | trifluoromethyl | hydrogen | Y¹ is CH, Y² is |
| | is trifluoromethyl | | | CH, Y³ is N |
| P.3056 | X¹ is chloro, X² is C—Cl, X³ | trifluoromethyl | methoxy | Y¹ is CH, Y² is |
| | is trifluoromethyl | | | CH, Y³ is N |
| P.3057 | X¹ is chloro, X² is C—Cl, X³ | trifluoromethyl | methyl | Y¹ is CH, Y² is |
| | is trifluoromethyl | | | CH, Y³ is N |
| P.3058 | X¹ is chloro, X² is C—Cl, X³ | trifluoromethyl | nitro | Y¹ is CH, Y² is |
| | is trifluoromethyl | | | CH, Y³ is N |
| P.3059 | X¹ is chloro, X² is C—Cl, X³ | trifluoromethyl | trifluoromethoxy | Y¹ is CH, Y² is |
| | is trifluoromethyl | | | CH, Y³ is N |
| P.3060 | X¹ is chloro, X² is C—Cl, X³ | trifluoromethyl | trifluoromethyl | Y¹ is CH, Y² is |
| | is trifluoromethyl | | | CH, Y³ is N |
| P.3061 | X¹ is trifluoromethyl, X² is | chlorodifluoromethyl | bromo | Y¹ is CH, Y² is |
| | CH, X³ is trifluoromethyl | | | CH, Y³ is N |
| P.3062 | X¹ is trifluoromethyl, X² is | chlorodifluoromethyl | chloro | Y¹ is CH, Y² is |
| | CH, X³ is trifluoromethyl | | | CH, Y³ is N |
| P.3063 | X¹ is trifluoromethyl, X² is | chlorodifluoromethyl | cyano | Y¹ is CH, Y² is |
| | CH, X³ is trifluoromethyl | | | CH, Y³ is N |
| P.3064 | X¹ is trifluoromethyl, X² is | chlorodifluoromethyl | cyclopropyl | Y¹ is CH, Y² is |
| | CH, X³ is trifluoromethyl | | | CH, Y³ is N |
| P.3065 | X¹ is trifluoromethyl, X² is | chlorodifluoromethyl | ethyl | Y¹ is CH, Y² is |
| | CH, X³ is trifluoromethyl | | | CH, Y³ is N |
| P.3066 | X¹ is trifluoromethyl, X² is | chlorodifluoromethyl | fluoro | Y¹ is CH, Y² is |
| | CH, X³ is trifluoromethyl | | | CH, Y³ is N |
| P.3067 | X¹ is trifluoromethyl, X² is | chlorodifluoromethyl | hydrogen | Y¹ is CH, Y² is |
| | CH, X³ is trifluoromethyl | | | CH, Y³ is N |
| P.3068 | X¹ is trifluoromethyl, X² is | chlorodifluoromethyl | methoxy | Y¹ is CH, Y² is |
| | CH, X³ is trifluoromethyl | | | CH, Y³ is N |
| P.3069 | X¹ is trifluoromethyl, X² is | chlorodifluoromethyl | methyl | Y¹ is CH, Y² is |
| | CH, X³ is trifluoromethyl | | | CH, Y³ is N |
| P.3070 | X¹ is trifluoromethyl, X² is | chlorodifluoromethyl | nitro | Y¹ is CH, Y² is |
| | CH, X³ is trifluoromethyl | | | CH, Y³ is N |
| P.3071 | X¹ is trifluoromethyl, X² is | chlorodifluoromethyl | trifluoromethoxy | Y¹ is CH, Y² is |
| | CH, X³ is trifluoromethyl | | | CH, Y³ is N |
| P.3072 | X¹ is trifluoromethyl, X² is | chlorodifluoromethyl | trifluoromethyl | Y¹ is CH, Y² is |
| | CH, X³ is trifluoromethyl | | | CH, Y³ is N |
| P.3073 | X¹ is trifluoromethyl, X² is | difluoromethyl | bromo | Y¹ is CH, Y² is |
| | CH, X³ is trifluoromethyl | | | CH, Y³ is N |
| P.3074 | X¹ is trifluoromethyl, X² is | difluoromethyl | chloro | Y¹ is CH, Y² is |
| | CH, X³ is trifluoromethyl | | | CH, Y³ is N |
| P.3075 | X¹ is trifluoromethyl, X² is | difluoromethyl | cyano | Y¹ is CH, Y² is |
| | CH, X³ is trifluoromethyl | | | CH, Y³ is N |
| P.3076 | X¹ is trifluoromethyl, X² is | difluoromethyl | cyclopropyl | Y¹ is CH, Y² is |
| | CH, X³ is trifluoromethyl | | | CH, Y³ is N |
| P.3077 | X¹ is trifluoromethyl, X² is | difluoromethyl | ethyl | Y¹ is CH, Y² is |
| | CH, X³ is trifluoromethyl | | | CH, Y³ is N |
| P.3078 | X¹ is trifluoromethyl, X² is | difluoromethyl | fluoro | Y¹ is CH, Y² is |
| | CH, X³ is trifluoromethyl | | | CH, Y³ is N |
| P.3079 | X¹ is trifluoromethyl, X² is | difluoromethyl | hydrogen | Y¹ is CH, Y² is |
| | CH, X³ is trifluoromethyl | | | CH, Y³ is N |
| P.3080 | X¹ is trifluoromethyl, X² is | difluoromethyl | methoxy | Y¹ is CH, Y² is |
| | CH, X³ is trifluoromethyl | | | CH, Y³ is N |
| P.3081 | X¹ is trifluoromethyl, X² is | difluoromethyl | methyl | Y¹ is CH, Y² is |
| | CH, X³ is trifluoromethyl | | | CH, Y³ is N |
| P.3082 | X¹ is trifluoromethyl, X² is | difluoromethyl | nitro | Y¹ is CH, Y² is |
| | CH, X³ is trifluoromethyl | | | CH, Y³ is N |
| P.3083 | X¹ is trifluoromethyl, X² is | difluoromethyl | trifluoromethoxy | Y¹ is CH, Y² is |
| | CH, X³ is trifluoromethyl | | | CH, Y³ is N |
| P.3084 | X¹ is trifluoromethyl, X² is | difluoromethyl | trifluoromethyl | Y¹ is CH, Y² is |
| | CH, X³ is trifluoromethyl | | | CH, Y³ is N |
| P.3085 | X¹ is trifluoromethyl, X² is | trifluoromethyl | bromo | Y¹ is CH, Y² is |
| | CH, X³ is trifluoromethyl | | | CH, Y³ is N |
| P.3086 | X¹ is trifluoromethyl, X² is | trifluoromethyl | chloro | Y¹ is CH, Y² is |
| | CH, X³ is trifluoromethyl | | | CH, Y³ is N |
| P.3087 | X¹ is trifluoromethyl, X² is | trifluoromethyl | cyano | Y¹ is CH, Y² is |
| | CH, X³ is trifluoromethyl | | | CH, Y³ is N |
| P.3088 | X¹ is trifluoromethyl, X² is | trifluoromethyl | cyclopropyl | Y¹ is CH, Y² is |
| | CH, X³ is trifluoromethyl | | | CH, Y³ is N |
| P.3089 | X¹ is trifluoromethyl, X² is | trifluoromethyl | ethyl | Y¹ is CH, Y² is |
| | CH, X³ is trifluoromethyl | | | CH, Y³ is N |
| P.3090 | X¹ is trifluoromethyl, X² is | trifluoromethyl | fluoro | Y¹ is CH, Y² is |
| | CH, X³ is trifluoromethyl | | | CH, Y³ is N |
| P.3091 | X¹ is trifluoromethyl, X² is | trifluoromethyl | hydrogen | Y¹ is CH, Y² is |
| | CH, X³ is trifluoromethyl | | | CH, Y³ is N |
| P.3092 | X¹ is trifluoromethyl, X² is | trifluoromethyl | methoxy | Y¹ is CH, Y² is |
| | CH, X³ is trifluoromethyl | | | CH, Y³ is N |
| P.3093 | X¹ is trifluoromethyl, X² is | trifluoromethyl | methyl | Y¹ is CH, Y² is |
| | CH, X³ is trifluoromethyl | | | CH, Y³ is N |
| P.3094 | X¹ is trifluoromethyl, X² is | trifluoromethyl | nitro | Y¹ is CH, Y² is |
| | CH, X³ is trifluoromethyl | | | CH, Y³ is N |
| P.3095 | X¹ is trifluoromethyl, X² is | trifluoromethyl | trifluoromethoxy | Y¹ is CH, Y² is |
| | CH, X³ is trifluoromethyl | | | CH, Y³ is N |
| P.3096 | X¹ is trifluoromethyl, X² is | trifluoromethyl | trifluoromethyl | Y¹ is CH, Y² is |
| | CH, X³ is trifluoromethyl | | | CH, Y³ is N |
| P.3097 | X¹ is trifluoromethyl, X² is | chlorodifluoromethyl | bromo | Y¹ is CH, Y² is |
| | CH, X³ is trifluoromethyl | | | CH, Y³ is N |
| P.3098 | X¹ is trifluoromethyl, X² is | chlorodifluoromethyl | chloro | Y¹ is CH, Y² is |
| | CH, X³ is trifluoromethyl | | | CH, Y³ is N |
| P.3099 | X¹ is trifluoromethyl, X² is | chlorodifluoromethyl | cyano | Y¹ is CH, Y² is |
| | CH, X³ is trifluoromethyl | | | CH, Y³ is N |
| P.3100 | X¹ is trifluoromethyl, X² is | chlorodifluoromethyl | cyclopropyl | Y¹ is CH, Y² is |
| | CH, X³ is trifluoromethyl | | | CH, Y³ is N |
| P.3101 | X¹ is trifluoromethyl, X² is | chlorodifluoromethyl | ethyl | Y¹ is CH, Y² is |
| | CH, X³ is trifluoromethyl | | | CH, Y³ is N |
| P.3102 | X¹ is trifluoromethyl, X² is | chlorodifluoromethyl | fluoro | Y¹ is CH, Y² is |
| | CH, X³ is trifluoromethyl | | | CH, Y³ is N |
| P.3103 | X¹ is trifluoromethyl, X² is | chlorodifluoromethyl | hydrogen | Y¹ is CH, Y² is |
| | CH, X³ is trifluoromethyl | | | CH, Y³ is N |
| P.3104 | X¹ is trifluoromethyl, X² is | chlorodifluoromethyl | methoxy | Y¹ is CH, Y² is |
| | CH, X³ is trifluoromethyl | | | CH, Y³ is N |
| P.3105 | X¹ is trifluoromethyl, X² is | chlorodifluoromethyl | methyl | Y¹ is CH, Y² is |
| | CH, X³ is trifluoromethyl | | | CH, Y³ is N |
| P.3106 | X¹ is trifluoromethyl, X² is | chlorodifluoromethyl | nitro | Y¹ is CH, Y² is |
| | CH, X³ is trifluoromethyl | | | CH, Y³ is N |
| P.3107 | X¹ is trifluoromethyl, X² is | chlorodifluoromethyl | trifluoromethoxy | Y¹ is CH, Y² is |
| | CH, X³ is trifluoromethyl | | | CH, Y³ is N |
| P.3108 | X¹ is trifluoromethyl, X² is | chlorodifluoromethyl | trifluoromethyl | Y¹ is CH, Y² is |
| | CH, X³ is trifluoromethyl | | | CH, Y³ is N |
| P.3109 | X¹ is trifluoromethyl, X² is | difluoromethyl | bromo | Y¹ is CH, Y² is |
| | CH, X³ is trifluoromethyl | | | CH, Y³ is N |
| P.3110 | X¹ is trifluoromethyl, X² is | difluoromethyl | chloro | Y¹ is CH, Y² is |
| | CH, X³ is trifluoromethyl | | | CH, Y³ is N |
| P.3111 | X¹ is trifluoromethyl, X² is | difluoromethyl | cyano | Y¹ is CH, Y² is |
| | CH, X³ is trifluoromethyl | | | CH, Y³ is N |
| P.3112 | X¹ is trifluoromethyl, X² is | difluoromethyl | cyclopropyl | Y¹ is CH, Y² is |
| | CH, X³ is trifluoromethyl | | | CH, Y³ is N |
| P.3113 | X¹ is trifluoromethyl, X² is | difluoromethyl | ethyl | Y¹ is CH, Y² is |
| | CH, X³ is trifluoromethyl | | | CH, Y³ is N |
| P.3114 | X¹ is trifluoromethyl, X² is | difluoromethyl | fluoro | Y¹ is CH, Y² is |
| | CH, X³ is trifluoromethyl | | | CH, Y³ is N |
| P.3115 | X¹ is trifluoromethyl, X² is | difluoromethyl | hydrogen | Y¹ is CH, Y² is |
| | CH, X³ is trifluoromethyl | | | CH, Y³ is N |
| P.3116 | X¹ is trifluoromethyl, X² is | difluoromethyl | methoxy | Y¹ is CH, Y² is |
| | CH, X³ is trifluoromethyl | | | CH, Y³ is N |
| P.3117 | X¹ is trifluoromethyl, X² is | difluoromethyl | methyl | Y¹ is CH, Y² is |
| | CH, X³ is trifluoromethyl | | | CH, Y³ is N |
| P.3118 | X¹ is trifluoromethyl, X² is | difluoromethyl | nitro | Y¹ is CH, Y² is |
| | CH, X³ is trifluoromethyl | | | CH, Y³ is N |
| P.3119 | X¹ is trifluoromethyl, X² is | difluoromethyl | trifluoromethoxy | Y¹ is CH, Y² is |
| | CH, X³ is trifluoromethyl | | | CH, Y³ is N |
| P.3120 | X¹ is trifluoromethyl, X² is | difluoromethyl | trifluoromethyl | Y¹ is CH, Y² is |
| | CH, X³ is trifluoromethyl | | | CH, Y³ is N |
| P.3121 | X¹ is trifluoromethyl, X² is | trifluoromethyl | bromo | Y¹ is CH, Y² is |
| | CH, X³ is trifluoromethyl | | | CH, Y³ is N |
| P.3122 | X¹ is trifluoromethyl, X² is | trifluoromethyl | chloro | Y¹ is CH, Y² is |
| | CH, X³ is trifluoromethyl | | | CH, Y³ is N |
| P.3123 | X¹ is trifluoromethyl, X² is | trifluoromethyl | cyano | Y¹ is CH, Y² is |
| | CH, X³ is trifluoromethyl | | | CH, Y³ is N |
| P.3124 | X¹ is trifluoromethyl, X² is | trifluoromethyl | cyclopropyl | Y¹ is CH, Y² is |
| | CH, X³ is trifluoromethyl | | | CH, Y³ is N |
| P.3125 | X¹ is trifluoromethyl, X² is | trifluoromethyl | ethyl | Y¹ is CH, Y² is |
| | CH, X³ is trifluoromethyl | | | CH, Y³ is N |
| P.3126 | X¹ is trifluoromethyl, X² is | trifluoromethyl | fluoro | Y¹ is CH, Y² is |
| | CH, X³ is trifluoromethyl | | | CH, Y³ is N |
| P.3127 | X¹ is trifluoromethyl, X² is | trifluoromethyl | hydrogen | Y¹ is CH, Y² is |
| | CH, X³ is trifluoromethyl | | | CH, Y³ is N |
| P.3128 | X¹ is trifluoromethyl, X² is | trifluoromethyl | methoxy | Y¹ is CH, Y² is |
| | CH, X³ is trifluoromethyl | | | CH, Y³ is N |
| P.3129 | X¹ is trifluoromethyl, X² is | trifluoromethyl | methyl | Y¹ is CH, Y² is |
| | CH, X³ is trifluoromethyl | | | CH, Y³ is N |
| P.3130 | X¹ is trifluoromethyl, X² is | trifluoromethyl | nitro | Y¹ is CH, Y² is |
| | CH, X³ is trifluoromethyl | | | CH, Y³ is N |
| P.3131 | X¹ is trifluoromethyl, X² is | trifluoromethyl | trifluoromethoxy | Y¹ is CH, Y² is |
| | CH, X³ is trifluoromethyl | | | CH, Y³ is N |
| P.3132 | X¹ is trifluoromethyl, X² is | trifluoromethyl | trifluoromethyl | Y¹ is CH, Y² is |
| | CH, X³ is trifluoromethyl | | | CH, Y³ is N |
| P.3133 | X¹ is trifluoromethyl, X² is | chlorodifluoromethyl | bromo | Y¹ is CH, Y² is |
| | CH, X³ is hydrogen | | | CH, Y³ is N |
| P.3134 | X¹ is trifluoromethyl, X² is | chlorodifluoromethyl | chloro | Y¹ is CH, Y² is |
| | CH, X³ is hydrogen | | | CH, Y³ is N |
| P.3135 | X¹ is trifluoromethyl, X² is | chlorodifluoromethyl | cyano | Y¹ is CH, Y² is |
| | CH, X³ is hydrogen | | | CH, Y³ is N |
| P.3136 | X¹ is trifluoromethyl, X² is | chlorodifluoromethyl | cyclopropyl | Y¹ is CH, Y² is |
| | CH, X³ is hydrogen | | | CH, Y³ is N |
| P.3137 | X¹ is trifluoromethyl, X² is | chlorodifluoromethyl | ethyl | Y¹ is CH, Y² is |
| | CH, X³ is hydrogen | | | CH, Y³ is N |
| P.3138 | X¹ is trifluoromethyl, X² is | chlorodifluoromethyl | fluoro | Y¹ is CH, Y² is |
| | CH, X³ is hydrogen | | | CH, Y³ is N |
| P.3139 | X¹ is trifluoromethyl, X² is | chlorodifluoromethyl | hydrogen | Y¹ is CH, Y² is |
| | CH, X³ is hydrogen | | | CH, Y³ is N |
| P.3140 | X¹ is trifluoromethyl, X² is | chlorodifluoromethyl | methoxy | Y¹ is CH, Y² is |
| | CH, X³ is hydrogen | | | CH, Y³ is N |
| P.3141 | X¹ is trifluoromethyl, X² is | chlorodifluoromethyl | methyl | Y¹ is CH, Y² is |
| | CH, X³ is hydrogen | | | CH, Y³ is N |
| P.3142 | X¹ is trifluoromethyl, X² is | chlorodifluoromethyl | nitro | Y¹ is CH, Y² is |
| | CH, X³ is hydrogen | | | CH, Y³ is N |
| P.3143 | X¹ is trifluoromethyl, X² is | chlorodifluoromethyl | trifluoromethoxy | Y¹ is CH, Y² is |
| | CH, X³ is hydrogen | | | CH, Y³ is N |
| P.3144 | X¹ is trifluoromethyl, X² is | chlorodifluoromethyl | trifluoromethyl | Y¹ is CH, Y² is |
| | CH, X³ is hydrogen | | | CH, Y³ is N |
| P.3145 | X¹ is trifluoromethyl, X² is | difluoromethyl | bromo | Y¹ is CH, Y² is |
| | CH, X³ is hydrogen | | | CH, Y³ is N |
| P.3146 | X¹ is trifluoromethyl, X² is | difluoromethyl | chloro | Y¹ is CH, Y² is |
| | CH, X³ is hydrogen | | | CH, Y³ is N |
| P.3147 | X¹ is trifluoromethyl, X² is | difluoromethyl | cyano | Y¹ is CH, Y² is |
| | CH, X³ is hydrogen | | | CH, Y³ is N |
| P.3148 | X¹ is trifluoromethyl, X² is | difluoromethyl | cyclopropyl | Y¹ is CH, Y² is |
| | CH, X³ is hydrogen | | | CH, Y³ is N |
| P.3149 | X¹ is trifluoromethyl, X² is | difluoromethyl | ethyl | Y¹ is CH, Y² is |
| | CH, X³ is hydrogen | | | CH, Y³ is N |
| P.3150 | X¹ is trifluoromethyl, X² is | difluoromethyl | fluoro | Y¹ is CH, Y² is |
| | CH, X³ is hydrogen | | | CH, Y³ is N |
| P.3151 | X¹ is trifluoromethyl, X² is | difluoromethyl | hydrogen | Y¹ is CH, Y² is |
| | CH, X³ is hydrogen | | | CH, Y³ is N |
| P.3152 | X¹ is trifluoromethyl, X² is | difluoromethyl | methoxy | Y¹ is CH, Y² is |
| | CH, X³ is hydrogen | | | CH, Y³ is N |
| P.3153 | X¹ is trifluoromethyl, X² is | difluoromethyl | methyl | Y¹ is CH, Y² is |
| | CH, X³ is hydrogen | | | CH, Y³ is N |
| P.3154 | X¹ is trifluoromethyl, X² is | difluoromethyl | nitro | Y¹ is CH, Y² is |
| | CH, X³ is hydrogen | | | CH, Y³ is N |
| P.3155 | X¹ is trifluoromethyl, X² is | difluoromethyl | trifluoromethoxy | Y¹ is CH, Y² is |
| | CH, X³ is hydrogen | | | CH, Y³ is N |
| P.3156 | X¹ is trifluoromethyl, X² is | difluoromethyl | trifluoromethyl | Y¹ is CH, Y² is |
| | CH, X³ is hydrogen | | | CH, Y³ is N |
| P.3157 | X¹ is trifluoromethyl, X² is | trifluoromethyl | bromo | Y¹ is CH, Y² is |
| | CH, X³ is hydrogen | | | CH, Y³ is N |
| P.3158 | X¹ is trifluoromethyl, X² is | trifluoromethyl | chloro | Y¹ is CH, Y² is |
| | CH, X³ is hydrogen | | | CH, Y³ is N |
| P.3159 | X¹ is trifluoromethyl, X² is | trifluoromethyl | cyano | Y¹ is CH, Y² is |
| | CH, X³ is hydrogen | | | CH, Y³ is N |
| P.3160 | X¹ is trifluoromethyl, X² is | trifluoromethyl | cyclopropyl | Y¹ is CH, Y² is |
| | CH, X³ is hydrogen | | | CH, Y³ is N |
| P.3161 | X¹ is trifluoromethyl, X² is | trifluoromethyl | ethyl | Y¹ is CH, Y² is |
| | CH, X³ is hydrogen | | | CH, Y³ is N |
| P.3162 | X¹ is trifluoromethyl, X² is | trifluoromethyl | fluoro | Y¹ is CH, Y² is |
| | CH, X³ is hydrogen | | | CH, Y³ is N |
| P.3163 | X¹ is trifluoromethyl, X² is | trifluoromethyl | hydrogen | Y¹ is CH, Y² is |
| | CH, X³ is hydrogen | | | CH, Y³ is N |
| P.3164 | X¹ is trifluoromethyl, X² is | trifluoromethyl | methoxy | Y¹ is CH, Y² is |
| | CH, X³ is hydrogen | | | CH, Y³ is N |
| P.3165 | X¹ is trifluoromethyl, X² is | trifluoromethyl | methyl | Y¹ is CH, Y² is |
| | CH, X³ is hydrogen | | | CH, Y³ is N |
| P.3166 | X¹ is trifluoromethyl, X² is | trifluoromethyl | nitro | Y¹ is CH, Y² is |
| | CH, X³ is hydrogen | | | CH, Y³ is N |
| P.3167 | X¹ is trifluoromethyl, X² is | trifluoromethyl | trifluoromethoxy | Y¹ is CH, Y² is |
| | CH, X³ is hydrogen | | | CH, Y³ is N |
| P.3168 | X¹ is trifluoromethyl, X² is | trifluoromethyl | trifluoromethyl | Y¹ is CH, Y² is |
| | CH, X³ is hydrogen | | | CH, Y³ is N |
| P.3169 | X¹ is chloro, X² is N, X³ is | chlorodifluoromethyl | bromo | Y¹ is CH, Y² is |
| | chloro | | | CH, Y³ is N |
| P.3170 | X¹ is chloro, X² is N, X³ is | chlorodifluoromethyl | chloro | Y¹ is CH, Y² is |
| | chloro | | | CH, Y³ is N |
| P.3171 | X¹ is chloro, X² is N, X³ is | chlorodifluoromethyl | cyano | Y¹ is CH, Y² is |
| | chloro | | | CH, Y³ is N |
| P.3172 | X¹ is chloro, X² is N, X³ is | chlorodifluoromethyl | cyclopropyl | Y¹ is CH, Y² is |
| | chloro | | | CH, Y³ is N |
| P.3173 | X¹ is chloro, X² is N, X³ is | chlorodifluoromethyl | ethyl | Y¹ is CH, Y² is |
| | chloro | | | CH, Y³ is N |
| P.3174 | X¹ is chloro, X² is N, X³ is | chlorodifluoromethyl | fluoro | Y¹ is CH, Y² is |
| | chloro | | | CH, Y³ is N |
| P.3175 | X¹ is chloro, X² is N, X³ is | chlorodifluoromethyl | hydrogen | Y¹ is CH, Y² is |
| | chloro | | | CH, Y³ is N |
| P.3176 | X¹ is chloro, X² is N, X³ is | chlorodifluoromethyl | methoxy | Y¹ is CH, Y² is |
| | chloro | | | CH, Y³ is N |
| P.3177 | X¹ is chloro, X² is N, X³ is | chlorodifluoromethyl | methyl | Y¹ is CH, Y² is |
| | chloro | | | CH, Y³ is N |
| P.3178 | X¹ is chloro, X² is N, X³ is | chlorodifluoromethyl | nitro | Y¹ is CH, Y² is |
| | chloro | | | CH, Y³ is N |
| P.3179 | X¹ is chloro, X² is N, X³ is | chlorodifluoromethyl | trifluoromethoxy | Y¹ is CH, Y² is |
| | chloro | | | CH, Y³ is N |
| P.3180 | X¹ is chloro, X² is N, X³ is | chlorodifluoromethyl | trifluoromethyl | Y¹ is CH, Y² is |
| | chloro | | | CH, Y³ is N |
| P.3181 | X¹ is chloro, X² is N, X³ is | difluoromethyl | bromo | Y¹ is CH, Y² is |
| | chloro | | | CH, Y³ is N |
| P.3182 | X¹ is chloro, X² is N, X³ is | difluoromethyl | chloro | Y¹ is CH, Y² is |
| | chloro | | | CH, Y³ is N |
| P.3183 | X¹ is chloro, X² is N, X³ is | difluoromethyl | cyano | Y¹ is CH, Y² is |
| | chloro | | | CH, Y³ is N |
| P.3184 | X¹ is chloro, X² is N, X³ is | difluoromethyl | cyclopropyl | Y¹ is CH, Y² is |
| | chloro | | | CH, Y³ is N |
| P.3185 | X¹ is chloro, X² is N, X³ is | difluoromethyl | ethyl | Y¹ is CH, Y² is |
| | chloro | | | CH, Y³ is N |
| P.3186 | X¹ is chloro, X² is N, X³ is | difluoromethyl | fluoro | Y¹ is CH, Y² is |
| | chloro | | | CH, Y³ is N |
| P.3187 | X¹ is chloro, X² is N, X³ is | difluoromethyl | hydrogen | Y¹ is CH, Y² is |
| | chloro | | | CH, Y³ is N |
| P.3188 | X¹ is chloro, X² is N, X³ is | difluoromethyl | methoxy | Y¹ is CH, Y² is |
| | chloro | | | CH, Y³ is N |
| P.3189 | X¹ is chloro, X² is N, X³ is | difluoromethyl | methyl | Y¹ is CH, Y² is |
| | chloro | | | CH, Y³ is N |
| P.3190 | X¹ is chloro, X² is N, X³ is | difluoromethyl | nitro | Y¹ is CH, Y² is |
| | chloro | | | CH, Y³ is N |
| P.3191 | X¹ is chloro, X² is N, X³ is | difluoromethyl | trifluoromethoxy | Y¹ is CH, Y² is |
| | chloro | | | CH, Y³ is N |
| P.3192 | X¹ is chloro, X² is N, X³ is | difluoromethyl | trifluoromethyl | Y¹ is CH, Y² is |
| | chloro | | | CH, Y³ is N |
| P.3193 | X¹ is chloro, X² is N, X³ is | trifluoromethyl | bromo | Y¹ is CH, Y² is |
| | chloro | | | CH, Y³ is N |
| P.3194 | X¹ is chloro, X² is N, X³ is | trifluoromethyl | chloro | Y¹ is CH, Y² is |
| | chloro | | | CH, Y³ is N |
| P.3195 | X¹ is chloro, X² is N, X³ is | trifluoromethyl | cyano | Y¹ is CH, Y² is |
| | chloro | | | CH, Y³ is N |
| P.3196 | X¹ is chloro, X² is N, X³ is | trifluoromethyl | cyclopropyl | Y¹ is CH, Y² is |
| | chloro | | | CH, Y³ is N |
| P.3197 | X¹ is chloro, X² is N, X³ is | trifluoromethyl | ethyl | Y¹ is CH, Y² is |
| | chloro | | | CH, Y³ is N |
| P.3198 | X¹ is chloro, X² is N, X³ is | trifluoromethyl | fluoro | Y¹ is CH, Y² is |
| | chloro | | | CH, Y³ is N |
| P.3199 | X¹ is chloro, X² is N, X³ is | trifluoromethyl | hydrogen | Y¹ is CH, Y² is |
| | chloro | | | CH, Y³ is N |
| P.3200 | X¹ is chloro, X² is N, X³ is | trifluoromethyl | methoxy | Y¹ is CH, Y² is |
| | chloro | | | CH, Y³ is N |
| P.3201 | X¹ is chloro, X² is N, X³ is | trifluoromethyl | methyl | Y¹ is CH, Y² is |
| | chloro | | | CH, Y³ is N |
| P.3202 | X¹ is chloro, X² is N, X³ is | trifluoromethyl | nitro | Y¹ is CH, Y² is |
| | chloro | | | CH, Y³ is N |
| P.3203 | X¹ is chloro, X² is N, X³ is | trifluoromethyl | trifluoromethoxy | Y¹ is CH, Y² is |
| | chloro | | | CH, Y³ is N |
| P.3204 | X¹ is chloro, X² is N, X³ is | trifluoromethyl | trifluoromethyl | Y¹ is CH, Y² is |
| | chloro | | | CH, Y³ is N |
| P.3205 | X¹ is trifluoromethyl, X² is | chlorodifluoromethyl | bromo | Y¹ is CH, Y² is |
| | N, X³ is trifluoromethyl | | | CH, Y³ is N |
| P.3206 | X¹ is trifluoromethyl, X² is | chlorodifluoromethyl | chloro | Y¹ is CH, Y² is |
| | N, X³ is trifluoromethyl | | | CH, Y³ is N |
| P.3207 | X¹ is trifluoromethyl, X² is | chlorodifluoromethyl | cyano | Y¹ is CH, Y² is |
| | N, X³ is trifluoromethyl | | | CH, Y³ is N |
| P.3208 | X¹ is trifluoromethyl, X² is | chlorodifluoromethyl | cyclopropyl | Y¹ is CH, Y² is |
| | N, X³ is trifluoromethyl | | | CH, Y³ is N |
| P.3209 | X¹ is trifluoromethyl, X² is | chlorodifluoromethyl | ethyl | Y¹ is CH, Y² is |
| | N, X³ is trifluoromethyl | | | CH, Y³ is N |
| P.3210 | X¹ is trifluoromethyl, X² is | chlorodifluoromethyl | fluoro | Y¹ is CH, Y² is |
| | N, X³ is trifluoromethyl | | | CH, Y³ is N |
| P.3211 | X¹ is trifluoromethyl, X² is | chlorodifluoromethyl | hydrogen | Y¹ is CH, Y² is |
| | N, X³ is trifluoromethyl | | | CH, Y³ is N |
| P.3212 | X¹ is trifluoromethyl, X² is | chlorodifluoromethyl | methoxy | Y¹ is CH, Y² is |
| | N, X³ is trifluoromethyl | | | CH, Y³ is N |
| P.3213 | X¹ is trifluoromethyl, X² is | chlorodifluoromethyl | methyl | Y¹ is CH, Y² is |
| | N, X³ is trifluoromethyl | | | CH, Y³ is N |
| P.3214 | X¹ is trifluoromethyl, X² is | chlorodifluoromethyl | nitro | Y¹ is CH, Y² is |
| | N, X³ is trifluoromethyl | | | CH, Y³ is N |
| P.3215 | X¹ is trifluoromethyl, X² is | chlorodifluoromethyl | trifluoromethoxy | Y¹ is CH, Y² is |
| | N, X³ is trifluoromethyl | | | CH, Y³ is N |
| P.3216 | X¹ is trifluoromethyl, X² is | chlorodifluoromethyl | trifluoromethyl | Y¹ is CH, Y² is |
| | N, X³ is trifluoromethyl | | | CH, Y³ is N |
| P.3217 | X¹ is trifluoromethyl, X² is | difluoromethyl | bromo | Y¹ is CH, Y² is |
| | N, X³ is trifluoromethyl | | | CH, Y³ is N |
| P.3218 | X¹ is trifluoromethyl, X² is | difluoromethyl | chloro | Y¹ is CH, Y² is |
| | N, X³ is trifluoromethyl | | | CH, Y³ is N |
| P.3219 | X¹ is trifluoromethyl, X² is | difluoromethyl | cyano | Y¹ is CH, Y² is |
| | N, X³ is trifluoromethyl | | | CH, Y³ is N |
| P.3220 | X¹ is trifluoromethyl, X² is | difluoromethyl | cyclopropyl | Y¹ is CH, Y² is |
| | N, X³ is trifluoromethyl | | | CH, Y³ is N |
| P.3221 | X¹ is trifluoromethyl, X² is | difluoromethyl | ethyl | Y¹ is CH, Y² is |
| | N, X³ is trifluoromethyl | | | CH, Y³ is N |
| P.3222 | X¹ is trifluoromethyl, X² is | difluoromethyl | fluoro | Y¹ is CH, Y² is |
| | N, X³ is trifluoromethyl | | | CH, Y³ is N |
| P.3223 | X¹ is trifluoromethyl, X² is | difluoromethyl | hydrogen | Y¹ is CH, Y² is |
| | N, X³ is trifluoromethyl | | | CH, Y³ is N |
| P.3224 | X¹ is trifluoromethyl, X² is | difluoromethyl | methoxy | Y¹ is CH, Y² is |
| | N, X³ is trifluoromethyl | | | CH, Y³ is N |
| P.3225 | X¹ is trifluoromethyl, X² is | difluoromethyl | methyl | Y¹ is CH, Y² is |
| | N, X³ is trifluoromethyl | | | CH, Y³ is N |
| P.3226 | X¹ is trifluoromethyl, X² is | difluoromethyl | nitro | Y¹ is CH, Y² is |
| | N, X³ is trifluoromethyl | | | CH, Y³ is N |
| P.3227 | X¹ is trifluoromethyl, X² is | difluoromethyl | trifluoromethoxy | Y¹ is CH, Y² is |
| | N, X³ is trifluoromethyl | | | CH, Y³ is N |
| P.3228 | X¹ is trifluoromethyl, X² is | difluoromethyl | trifluoromethyl | Y¹ is CH, Y² is |
| | N, X³ is trifluoromethyl | | | CH, Y³ is N |
| P.3229 | X¹ is trifluoromethyl, X² is | trifluoromethyl | bromo | Y¹ is CH, Y² is |
| | N, X³ is trifluoromethyl | | | CH, Y³ is N |
| P.3230 | X¹ is trifluoromethyl, X² is | trifluoromethyl | chloro | Y¹ is CH, Y² is |
| | N, X³ is trifluoromethyl | | | CH, Y³ is N |
| P.3231 | X¹ is trifluoromethyl, X² is | trifluoromethyl | cyano | Y¹ is CH, Y² is |
| | N, X³ is trifluoromethyl | | | CH, Y³ is N |
| P.3232 | X¹ is trifluoromethyl, X² is | trifluoromethyl | cyclopropyl | Y¹ is CH, Y² is |
| | N, X³ is trifluoromethyl | | | CH, Y³ is N |
| P.3233 | X¹ is trifluoromethyl, X² is | trifluoromethyl | ethyl | Y¹ is CH, Y² is |
| | N, X³ is trifluoromethyl | | | CH, Y³ is N |
| P.3234 | X¹ is trifluoromethyl, X² is | trifluoromethyl | fluoro | Y¹ is CH, Y² is |
| | N, X³ is trifluoromethyl | | | CH, Y³ is N |
| P.3235 | X¹ is trifluoromethyl, X² is | trifluoromethyl | hydrogen | Y¹ is CH, Y² is |
| | N, X³ is trifluoromethyl | | | CH, Y³ is N |
| P.3236 | X¹ is trifluoromethyl, X² is | trifluoromethyl | methoxy | Y¹ is CH, Y² is |
| | N, X³ is trifluoromethyl | | | CH, Y³ is N |
| P.3237 | X¹ is trifluoromethyl, X² is | trifluoromethyl | methyl | Y¹ is CH, Y² is |
| | N, X³ is trifluoromethyl | | | CH, Y³ is N |
| P.3238 | X¹ is trifluoromethyl, X² is | trifluoromethyl | nitro | Y¹ is CH, Y² is |
| | N, X³ is trifluoromethyl | | | CH, Y³ is N |
| P.3239 | X¹ is trifluoromethyl, X² is | trifluoromethyl | trifluoromethoxy | Y¹ is CH, Y² is |
| | N, X³ is trifluoromethyl | | | CH, Y³ is N |
| P.3240 | X¹ is trifluoromethyl, X² is | trifluoromethyl | trifluoromethyl | Y¹ is CH, Y² is |
| | N, X³ is trifluoromethyl | | | CH, Y³ is N |

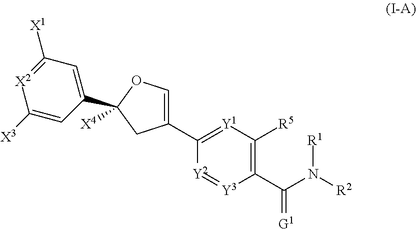

### Table 1P:

Table 1P provides 3240 compounds of formula (I-A) wherein G¹ is oxygen, R¹ is hydrogen, R² is G.001, X¹, X², X³, Y¹, Y², Y³, X⁴ and R⁵ have the values listed in the table P.

### Table 2P:

Table 2P provides 3240 compounds of formula (I-A) wherein G¹ is oxygen, R¹ is hydrogen, R² is G.002, X¹, X², X³, Y¹, Y², Y³, X⁴ and R⁵ have the values listed in the table P.

### Table 3P:

Table 3P provides 3240 compounds of formula (I-A) wherein G¹ is oxygen, R¹ is hydrogen, R² is G.003, X¹, X², X³, Y¹, Y², Y³, X⁴ and R⁵ have the values listed in the table P.

### Table 4P:

Table 4P provides 3240 compounds of formula (I-A) wherein G¹ is oxygen, R¹ is hydrogen, R² is G.004, X¹, X², X³, Y¹, Y², Y³, X⁴ and R⁵ have the values listed in the table P.

### Table 5P:

Table 5P provides 3240 compounds of formula (I-A) wherein G¹ is oxygen, R¹ is hydrogen, R² is G.005, X¹, X², X³, Y¹, Y², Y³, X⁴ and R⁵ have the values listed in the table P.

### Table 6P:

Table 6P provides 3240 compounds of formula (I-A) wherein G¹ is oxygen, R¹ is hydrogen, R² is G.006, X¹, X², X³, Y¹, Y², Y³, X⁴ and R⁵ have the values listed in the table P.

### Table 7P:

Table 7P provides 3240 compounds of formula (I-A) wherein G¹ is oxygen, R¹ is hydrogen, R² is G.007, X¹, X², X³, Y¹, Y², Y³, X⁴ and R⁵ have the values listed in the table P.

### Table 8P:

Table 8P provides 3240 compounds of formula (I-A) wherein G¹ is oxygen, R¹ is hydrogen, R² is G.008, X¹, X², X³, Y¹, Y², Y³, X⁴ and R⁵ have the values listed in the table P.

### Table 9P:

Table 9P provides 3240 compounds of formula (I-A) wherein G¹ is oxygen, R¹ is hydrogen, R² is G.009, X¹, X², X³, Y¹, Y², Y³, X⁴ and R⁵ have the values listed in the table P.

### Table 10P:

Table 10P provides 3240 compounds of formula (I-A) wherein G¹ is oxygen, R¹ is hydrogen, R² is G.010, X¹, X², X³, Y¹, Y², Y³, X⁴ and R⁵ have the values listed in the table P.

### Table 11P:

Table 11P provides 3240 compounds of formula (I-A) wherein G¹ is oxygen, R¹ is hydrogen, R² is G.011, X¹, X², X³, Y¹, Y², Y³, X⁴ and R⁵ have the values listed in the table P.

### Table 12P:

Table 12P provides 3240 compounds of formula (I-A) wherein G¹ is oxygen, R¹ is hydrogen, R² is G.012, X¹, X², X³, Y¹, Y², Y³, X⁴ and R⁵ have the values listed in the table P.

### Table 13P:

Table 13P provides 3240 compounds of formula (I-A) wherein G¹ is oxygen, R¹ is hydrogen, R² is G.013, X¹, X², X³, Y¹, Y², Y³, X⁴ and R⁵ have the values listed in the table P.

### Table 14P:

Table 14P provides 3240 compounds of formula (I-A) wherein G¹ is oxygen, R¹ is hydrogen, R² is G.014, X¹, X², X³, Y¹, Y², Y³, X⁴ and R⁵ have the values listed in the table P.

### Table 15P:

Table 15P provides 3240 compounds of formula (I-A) wherein G¹ is oxygen, R¹ is hydrogen, R² is G.015, X¹, X², X³, Y¹, Y², Y³, X⁴ and R⁵ have the values listed in the table P.

### Table 16P:

Table 16P provides 3240 compounds of formula (I-A) wherein G¹ is oxygen, R¹ is hydrogen, R² is G.016, X¹, X², X³, Y¹, Y², Y³, X⁴ and R⁵ have the values listed in the table P.

### Table 17P:

Table 17P provides 3240 compounds of formula (I-A) wherein G¹ is oxygen, R¹ is hydrogen, R² is G.017, X¹, X², X³, Y¹, Y², Y³, X⁴ and R⁵ have the values listed in the table P.

### Table 18P:

Table 18P provides 3240 compounds of formula (I-A) wherein G¹ is oxygen, R¹ is hydrogen, R² is G.018, X¹, X², X³, Y¹, Y², Y³, X⁴ and R⁵ have the values listed in the table P.

### Table 19P:

Table 19P provides 3240 compounds of formula (I-A) wherein G¹ is oxygen, R¹ is hydrogen, R² is G.019, X¹, X², X³, Y¹, Y², Y³, X⁴ and R⁵ have the values listed in the table P.

### Table 20P:

Table 20P provides 3240 compounds of formula (I-A) wherein G¹ is oxygen, R¹ is hydrogen, R² is G.020, X¹, X², X³, Y¹, Y², Y³, X⁴ and R⁵ have the values listed in the table P.

### Table 21P:

Table 21P provides 3240 compounds of formula (I-A) wherein G¹ is oxygen, R¹ is hydrogen, R² is G.021, X¹, X², X³, Y¹, Y², Y³, X⁴ and R⁵ have the values listed in the table P.

### Table 22P:

Table 22P provides 3240 compounds of formula (I-A) wherein G¹ is oxygen, R¹ is hydrogen, R² is G.022, X¹, X², X³, Y¹, Y², Y³, X⁴ and R⁵ have the values listed in the table P.

### Table 23P:

Table 23P provides 3240 compounds of formula (I-A) wherein G¹ is oxygen, R¹ is hydrogen, R² is G.023, X¹, X², X³, Y¹, Y², Y³, X⁴ and R⁵ have the values listed in the table P.

### Table 24P:

Table 24P provides 3240 compounds of formula (I-A) wherein G¹ is oxygen, R¹ is hydrogen, R² is G.024, X¹, X², X³, Y¹, Y², Y³, X⁴ and R⁵ have the values listed in the table P.

### Table 25P:

Table 25P provides 3240 compounds of formula (I-A) wherein G¹ is oxygen, R¹ is hydrogen, R² is G.025, X¹, X², X³, Y¹, Y², Y³, X⁴ and R⁵ have the values listed in the table P.

### Table 26P:

Table 26P provides 3240 compounds of formula (I-A) wherein G¹ is oxygen, R¹ is hydrogen, R² is G.026, X¹, X², X³, Y¹, Y², Y³, X⁴ and R⁵ have the values listed in the table P.

### Table 27P:

Table 27P provides 3240 compounds of formula (I-A) wherein G¹ is oxygen, R¹ is hydrogen, R² is G.027, X¹, X², X³, Y¹, Y², Y³, X⁴ and R⁵ have the values listed in the table P.

### Table 28P:

Table 28P provides 3240 compounds of formula (I-A) wherein G¹ is oxygen, R¹ is hydrogen, R² is G.028, X¹, X², X³, Y¹, Y², Y³, X⁴ and R⁵ have the values listed in the table P.

### Table 29P:

Table 29P provides 3240 compounds of formula (I-A) wherein G¹ is oxygen, R¹ is hydrogen, R² is G.029, X¹, X², X³, Y¹, Y², Y³, X⁴ and R⁵ have the values listed in the table P.

### Table 30P:

Table 30P provides 3240 compounds of formula (I-A) wherein G¹ is oxygen, R¹ is hydrogen, R² is G.030, X¹, X², X³, Y¹, Y², Y³, X⁴ and R⁵ have the values listed in the table P.

### Table 31P:

Table 31P provides 3240 compounds of formula (I-A) wherein G¹ is oxygen, R¹ is hydrogen, R² is G.031, X¹, X², X³, Y¹, Y², Y³, X⁴ and R⁵ have the values listed in the table P.

### Table 32P:

Table 32P provides 3240 compounds of formula (I-A) wherein G¹ is oxygen, R¹ is hydrogen, R² is G.032, X¹, X², X³, Y¹, Y², Y³, X⁴ and R⁵ have the values listed in the table P.

### Table 33P:

Table 33P provides 3240 compounds of formula (I-A) wherein G¹ is oxygen, R¹ is hydrogen, R² is G.033, X¹, X², X³, Y¹, Y², Y³, X⁴ and R⁵ have the values listed in the table P.

### Table 34P:

Table 34P provides 3240 compounds of formula (I-A) wherein G¹ is oxygen, R¹ is hydrogen, R² is G.034, X¹, X², X³, Y¹, Y², Y³, X⁴ and R⁵ have the values listed in the table P.

### Table 35P:

Table 35P provides 3240 compounds of formula (I-A) wherein G¹ is oxygen, R¹ is hydrogen, R² is G.035, X¹, X², X³, Y¹, Y², Y³, X⁴ and R⁵ have the values listed in the table P.

### Table 36P:

Table 36P provides 3240 compounds of formula (I-A) wherein G¹ is oxygen, R¹ is hydrogen, R² is G.036, X¹, X², X³, Y¹, Y², Y³, X⁴ and R⁵ have the values listed in the table P.

### Table 37P:

Table 37P provides 3240 compounds of formula (I-A) wherein G¹ is oxygen, R¹ is hydrogen, R² is G.037, X¹, X², X³, Y¹, Y², Y³, X⁴ and R⁵ have the values listed in the table P.

### Table 38P:

Table 38P provides 3240 compounds of formula (I-A) wherein G¹ is oxygen, R¹ is hydrogen, R² is G.038, X¹, X², X³, Y¹, Y², Y³, X⁴ and R⁵ have the values listed in the table P.

### Table 39P:

Table 39P provides 3240 compounds of formula (I-A) wherein G¹ is oxygen, R¹ is hydrogen, R² is G.039, X¹, X², X³, Y¹, Y², Y³, X⁴ and R⁵ have the values listed in the table P.

### Table 40P:

Table 40P provides 3240 compounds of formula (I-A) wherein G¹ is oxygen, R¹ is hydrogen, R² is G.040, X¹, X², X³, Y¹, Y², Y³, X⁴ and R⁵ have the values listed in the table P.

### Table 41P:

Table 41P provides 3240 compounds of formula (I-A) wherein G¹ is oxygen, R¹ is hydrogen, R² is G.041, X¹, X², X³, Y¹, Y², Y³, X⁴ and R⁵ have the values listed in the table P.

### Table 42P:

Table 42P provides 3240 compounds of formula (I-A) wherein G¹ is oxygen, R¹ is hydrogen, R² is G.042, X¹, X², X³, Y¹, Y², Y³, X⁴ and R⁵ have the values listed in the table P.

### Table 43P:

Table 43P provides 3240 compounds of formula (I-A) wherein G¹ is oxygen, R¹ is hydrogen, R² is G.043, X¹, X², X³, Y¹, Y², Y³, X⁴ and R⁵ have the values listed in the table P.

### Table 44P:

Table 44P provides 3240 compounds of formula (I-A) wherein G¹ is oxygen, R¹ is hydrogen, R² is G.044, X¹, X², X³, Y¹, Y², Y³, X⁴ and R⁵ have the values listed in the table P.

### Table 45P:

Table 45P provides 3240 compounds of formula (I-A) wherein G¹ is oxygen, R¹ is hydrogen, R² is G.045, X¹, X², X³, Y¹, Y², Y³, X⁴ and R⁵ have the values listed in the table P.

### Table 46P:

Table 46P provides 3240 compounds of formula (I-A) wherein G¹ is oxygen, R¹ is hydrogen, R² is G.046, X¹, X², X³, Y¹, Y², Y³, X⁴ and R⁵ have the values listed in the table P.

### Table 47P:

Table 47P provides 3240 compounds of formula (I-A) wherein G¹ is oxygen, R¹ is hydrogen, R² is G.047, X¹, X², X³, Y¹, Y², Y³, X⁴ and R⁵ have the values listed in the table P.

### Table 48P:

Table 48P provides 3240 compounds of formula (I-A) wherein G¹ is oxygen, R¹ is hydrogen, R² is G.048, X¹, X², X³, Y¹, Y², Y³, X⁴ and R⁵ have the values listed in the table P.

### Table 49P:

Table 49P provides 3240 compounds of formula (I-A) wherein G¹ is oxygen, R¹ is hydrogen, R² is G.049, X¹, X², X³, Y¹, Y², Y³, X⁴ and R⁵ have the values listed in the table P.

### Table 50P:

Table 50P provides 3240 compounds of formula (I-A) wherein G¹ is oxygen, R¹ is hydrogen, R² is G.050, X¹, X², X³, Y¹, Y², Y³, X⁴ and R⁵ have the values listed in the table P.

### Table 51P:

Table 51P provides 3240 compounds of formula (I-A) wherein G¹ is oxygen, R¹ is hydrogen, R² is G.051, X¹, X², X³, Y¹, Y², Y³, X⁴ and R⁵ have the values listed in the table P.

### Table 52P:

Table 52P provides 3240 compounds of formula (I-A) wherein G¹ is oxygen, R¹ is hydrogen, R² is G.052, X¹, X², X³, Y¹, Y², Y³, X⁴ and R⁵ have the values listed in the table P.

### Table 53P:

Table 53P provides 3240 compounds of formula (I-A) wherein G¹ is oxygen, R¹ is hydrogen, R² is G.053, X¹, X², X³, Y¹, Y², Y³, X⁴ and R⁵ have the values listed in the table P.

### Table 54P:

Table 54P provides 3240 compounds of formula (I-A) wherein G¹ is oxygen, R¹ is hydrogen, R² is G.054, X¹, X², X³, Y¹, Y², Y³, X⁴ and R⁵ have the values listed in the table P.

### Table 55P:

Table 55P provides 3240 compounds of formula (I-A) wherein G¹ is oxygen, R¹ is hydrogen, R² is G.055, X¹, X², X³, Y¹, Y², Y³, X⁴ and R⁵ have the values listed in the table P.

### Table 56P:

Table 56P provides 3240 compounds of formula (I-A) wherein G¹ is oxygen, R¹ is hydrogen, R² is G.056, X¹, X², X³, Y¹, Y², Y³, X⁴ and R⁵ have the values listed in the table P.

### Table 57P:

Table 57P provides 3240 compounds of formula (I-A) wherein G¹ is oxygen, R¹ is hydrogen, R² is G.057, X¹, X², X³, Y¹, Y², Y³, X⁴ and R⁵ have the values listed in the table P.

### Table 58P:

Table 58P provides 3240 compounds of formula (I-A) wherein G¹ is oxygen, R¹ is hydrogen, R² is G.058, X¹, X², X³, Y¹, Y², Y³, X⁴ and R⁵ have the values listed in the table P.

### Table 59P:

Table 59P provides 3240 compounds of formula (I-A) wherein G¹ is oxygen, R¹ is hydrogen, R² is G.059, X¹, X², X³, Y¹, Y², Y³, X⁴ and R⁵ have the values listed in the table P.

### Table 60P:

Table 60P provides 3240 compounds of formula (I-A) wherein G¹ is oxygen, R¹ is hydrogen, R² is G.060, X¹, X², X³, Y¹, Y², Y³, X⁴ and R⁵ have the values listed in the table P.

### Table 61P:

Table 61P provides 3240 compounds of formula (I-A) wherein G¹ is oxygen, R¹ is hydrogen, R² is G.061, X¹, X², X³, Y¹, Y², Y³, X⁴ and R⁵ have the values listed in the table P.

### Table 62P:

Table 62P provides 3240 compounds of formula (I-A) wherein G¹ is oxygen, R¹ is hydrogen, R² is G.062, X¹, X², X³, Y¹, Y², Y³, X⁴ and R⁵ have the values listed in the table P.

### Table 63P:

Table 63P provides 3240 compounds of formula (I-A) wherein G¹ is oxygen, R¹ is hydrogen, R² is G.063, X¹, X², X³, Y¹, Y², Y³, X⁴ and R⁵ have the values listed in the table P.

### Table 64P:

Table 64P provides 3240 compounds of formula (I-A) wherein G¹ is oxygen, R¹ is hydrogen, R² is G.064, X¹, X², X³, Y¹, Y², Y³, X⁴ and R⁵ have the values listed in the table P.

### Table 65P:

Table 65P provides 3240 compounds of formula (I-A) wherein G¹ is oxygen, R¹ is hydrogen, R² is G.065, X¹, X², X³, Y¹, Y², Y³, X⁴ and R⁵ have the values listed in the table P.

### Table 66P:

Table 66P provides 3240 compounds of formula (I-A) wherein G¹ is oxygen, R¹ is hydrogen, R² is G.066, X¹, X², X³, Y¹, Y², Y³, X⁴ and R⁵ have the values listed in the table P.

### Table 67P:

Table 67P provides 3240 compounds of formula (I-A) wherein G¹ is oxygen, R¹ is hydrogen, R² is G.067, X¹, X², X³, Y¹, Y², Y³, X⁴ and R⁵ have the values listed in the table P.

### Table 68P:

Table 68P provides 3240 compounds of formula (I-A) wherein G¹ is oxygen, R¹ is hydrogen, R² is G.068, X¹, X², X³, Y¹, Y², Y³, X⁴ and R⁵ have the values listed in the table P.

### Table 69P:

Table 69P provides 3240 compounds of formula (I-A) wherein G¹ is oxygen, R¹ is hydrogen, R² is G.069, X¹, X², X³, Y¹, Y², Y³, X⁴ and R⁵ have the values listed in the table P.

### Table 70P:

Table 70P provides 3240 compounds of formula (I-A) wherein G¹ is oxygen, R¹ is hydrogen, R² is G.070, X¹, X², X³, Y¹, Y², Y³, X⁴ and R⁵ have the values listed in the table P.

### Table 71P:

Table 71P provides 3240 compounds of formula (I-A) wherein G¹ is oxygen, R¹ is hydrogen, R² is G.071, X¹, X², X³, Y¹, Y², Y³, X⁴ and R⁵ have the values listed in the table P.

### Table 72P:

Table 72P provides 3240 compounds of formula (I-A) wherein G¹ is oxygen, R¹ is hydrogen, R² is G.072, X¹, X², X³, Y¹, Y², Y³, X⁴ and R⁵ have the values listed in the table P.

### Table 73P:

Table 73P provides 3240 compounds of formula (I-A) wherein G¹ is oxygen, R¹ is hydrogen, R² is G.073, X¹, X², X³, Y¹, Y², Y³, X⁴ and R⁵ have the values listed in the table P.

### Table 74P:

Table 74P provides 3240 compounds of formula (I-A) wherein G¹ is oxygen, R¹ is hydrogen, R² is G.074, X¹, X², X³, Y¹, Y², Y³, X⁴ and R⁵ have the values listed in the table P.

### Table 75P:

Table 75P provides 3240 compounds of formula (I-A) wherein G¹ is oxygen, R¹ is hydrogen, R² is G.075, X¹, X², X³, Y¹, Y², Y³, X⁴ and R⁵ have the values listed in the table P.

### Table 76P:

Table 76P provides 3240 compounds of formula (I-A) wherein G¹ is oxygen, R¹ is hydrogen, R² is G.076, X¹, X², X³, Y¹, Y², Y³, X⁴ and R⁵ have the values listed in the table P.

### Table 77P:

Table 77P provides 3240 compounds of formula (I-A) wherein G¹ is oxygen, R¹ is hydrogen, R² is G.077, X¹, X², X³, Y¹, Y², Y³, X⁴ and R⁵ have the values listed in the table P.

### Table 78P:

Table 78P provides 3240 compounds of formula (I-A) wherein G¹ is oxygen, R¹ is hydrogen, R² is G.078, X¹, X², X³, Y¹, Y², Y³, X⁴ and R⁵ have the values listed in the table P.

### Table 79P:

Table 79P provides 3240 compounds of formula (I-A) wherein G¹ is oxygen, R¹ is hydrogen, R² is G.079, X¹, X², X³, Y¹, Y², Y³, X⁴ and R⁵ have the values listed in the table P.

### Table 80P:

Table 80P provides 3240 compounds of formula (I-A) wherein G¹ is oxygen, R¹ is hydrogen, R² is G.080, X¹, X², X³, Y¹, Y², Y³, X⁴ and R⁵ have the values listed in the table P.

### Table 81P:

Table 81P provides 3240 compounds of formula (I-A) wherein G¹ is oxygen, R¹ is hydrogen, R² is G.081, X¹, X², X³, Y¹, Y², Y³, X⁴ and R⁵ have the values listed in the table P.

### Table 82P:

Table 82P provides 3240 compounds of formula (I-A) wherein G¹ is oxygen, R¹ is hydrogen, R² is G.082, X¹, X², X³, Y¹, Y², Y³, X⁴ and R⁵ have the values listed in the table P.

### Table 83P:

Table 83P provides 3240 compounds of formula (I-A) wherein G¹ is oxygen, R¹ is hydrogen, R² is G.083, X¹, X², X³, Y¹, Y², Y³, X⁴ and R⁵ have the values listed in the table P.

### Table 84P:

Table 84P provides 3240 compounds of formula (I-A) wherein G¹ is oxygen, R¹ is hydrogen, R² is G.084, X¹, X², X³, Y¹, Y², Y³, X⁴ and R⁵ have the values listed in the table P.

### Table 85P:

Table 85P provides 3240 compounds of formula (I-A) wherein G¹ is oxygen, R¹ is hydrogen, R² is G.085, X¹, X², X³, Y¹, Y², Y³, X⁴ and R⁵ have the values listed in the table P.

### Table 86P:

Table 86P provides 3240 compounds of formula (I-A) wherein G¹ is oxygen, R¹ is hydrogen, R² is G.086, X¹, X², X³, Y¹, Y², Y³, X⁴ and R⁵ have the values listed in the table P.

### Table 87P:

Table 87P provides 3240 compounds of formula (I-A) wherein G¹ is oxygen, R¹ is hydrogen, R² is G.087, X¹, X², X³, Y¹, Y², Y³, X⁴ and R⁵ have the values listed in the table P.

### Table 88P:

Table 88P provides 3240 compounds of formula (I-A) wherein G¹ is oxygen, R¹ is hydrogen, R² is G.088, X¹, X², X³, Y¹, Y², Y³, X⁴ and R⁵ have the values listed in the table P.

### Table 89P:

Table 89P provides 3240 compounds of formula (I-A) wherein G¹ is oxygen, R¹ is hydrogen, R² is G.089, X¹, X², X³, Y¹, Y², Y³, X⁴ and R⁵ have the values listed in the table P.

### Table 90P:

Table 90P provides 3240 compounds of formula (I-A) wherein G¹ is oxygen, R¹ is hydrogen, R² is G.090, X¹, X², X³, Y¹, Y², Y³, X⁴ and R⁵ have the values listed in the table P.

### Table 91P

Table 91P provides 3240 compounds of formula (I-A) wherein G¹ is oxygen, R¹ is hydrogen, R² is G.091, X¹, X², X³, Y¹, Y², Y³, X⁴ and R⁵ have the values listed in the table P.

### Table 92P

Table 92P provides 3240 compounds of formula (I-A) wherein G¹ is oxygen, R¹ is hydrogen, R² is G.092, X¹, X², X³, Y¹, Y², Y³, X⁴ and R⁵ have the values listed in the table P.

### Table 93P

Table 93P provides 3240 compounds of formula (I-A) wherein G¹ is oxygen, R¹ is hydrogen, R² is G.093, X¹, X², X³, Y¹, Y², Y³, X⁴ and R⁵ have the values listed in the table P.

### Table 94P

Table 94P provides 3240 compounds of formula (I-A) wherein G¹ is oxygen, R¹ is hydrogen, R² is G.094, X¹, X², X³, Y¹, Y², Y³, X⁴ and R⁵ have the values listed in the table P.

### Table 95P

Table 95P provides 3240 compounds of formula (I-A) wherein G¹ is oxygen, R¹ is hydrogen, R² is G.095, X¹, X², X³, Y¹, Y², Y³, X⁴ and R⁵ have the values listed in the table P.

### Table 96P

Table 96P provides 3240 compounds of formula (I-A) wherein G¹ is oxygen, R¹ is hydrogen, R² is G.096, X¹, X², X³, Y¹, Y², Y³, X⁴ and R⁵ have the values listed in the table P.

### Table 97P

Table 97P provides 3240 compounds of formula (I-A) wherein G¹ is oxygen, R¹ is hydrogen, R² is G.097, X¹, X², X³, Y¹, Y², Y³, X⁴ and R⁵ have the values listed in the table P.

### Table 98P

Table 98P provides 3240 compounds of formula (I-A) wherein G¹ is oxygen, R¹ is hydrogen, R² is G.098, X¹, X², X³, Y¹, Y², Y³, X⁴ and R⁵ have the values listed in the table P.

### Table 99P

Table 99P provides 3240 compounds of formula (I-A) wherein G¹ is oxygen, R¹ is hydrogen, R² is G.099, X¹, X², X³, Y¹, Y², Y³, X⁴ and R⁵ have the values listed in the table P.

### Table 100P

Table 100P provides 3240 compounds of formula (I-A) wherein G¹ is oxygen, R¹ is hydrogen, R² is G.100, X¹, X², X³, Y¹, Y², Y³, X⁴ and R⁵ have the values listed in the table P.

### Table 101P

Table 101P provides 3240 compounds of formula (I-A) wherein G¹ is oxygen, R¹ is hydrogen, R² is G.101, X¹, X², X³, Y¹, Y², Y³, X⁴ and R⁵ have the values listed in the table P.

### Table 102P

Table 102P provides 3240 compounds of formula (I-A) wherein G¹ is oxygen, R¹ is hydrogen, R² is G.102, X¹, X², X³, Y¹, Y², Y³, X⁴ and R⁵ have the values listed in the table P.

### Table 103P

Table 103P provides 3240 compounds of formula (I-A) wherein G¹ is oxygen, R¹ is hydrogen, R² is G.103, X¹, X², X³, Y¹, Y², Y³, X⁴ and R⁵ have the values listed in the table P.

### Table 104P

Table 104P provides 3240 compounds of formula (I-A) wherein G¹ is oxygen, R¹ is hydrogen, R² is G.104, X¹, X², X³, Y¹, Y², Y³, X⁴ and R⁵ have the values listed in the table P.

### Table 105P

Table 105P provides 3240 compounds of formula (I-A) wherein G¹ is oxygen, R¹ is hydrogen, R² is G.105, X¹, X², X³, Y¹, Y², Y³, X⁴ and R⁵ have the values listed in the table P.

### Table 106P

Table 106P provides 3240 compounds of formula (I-A) wherein G¹ is oxygen, R¹ is hydrogen, R² is G.106, X¹, X², X³, Y¹, Y², Y³, X⁴ and R⁵ have the values listed in the table P.

### Table 107P

Table 107P provides 3240 compounds of formula (I-A) wherein G¹ is oxygen, R¹ is hydrogen, R² is G.107, X¹, X², X³, Y¹, Y², Y³, X⁴ and R⁵ have the values listed in the table P.

### Table 108P

Table 108P provides 3240 compounds of formula (I-A) wherein G¹ is oxygen, R¹ is hydrogen, R² is G.108, X¹, X², X³, Y¹, Y², Y³, X⁴ and R⁵ have the values listed in the table P.

### Table 109P

Table 109P provides 3240 compounds of formula (I-A) wherein G¹ is oxygen, R¹ is hydrogen, R² is G.109, X¹, X², X³, Y¹, Y², Y³, X⁴ and R⁵ have the values listed in the table P.

### Table 110P

Table 110P provides 3240 compounds of formula (I-A) wherein G¹ is oxygen, R¹ is hydrogen, R² is G.110, X¹, X², X³, Y¹, Y², Y³, X⁴ and R⁵ have the values listed in the table P.

### Table 111P:

Table 111P provides 3240 compounds of formula (I-A) wherein G¹ is oxygen, R¹ is hydrogen, R² is G.111, X¹, X², X³, Y¹, Y², Y³, X⁴ and R⁵ have the values listed in the table P.

### Table 112 P

Table 112P provides 3240 compounds of formula (I-A) wherein G¹ is oxygen, R¹ is hydrogen, R² is G.112, X¹, X², X³, Y¹, Y², Y³, X⁴ and R⁵ have the values listed in the table P.

### Table 113 P

Table 113P provides 3240 compounds of formula (I-A) wherein G¹ is oxygen, R¹ is hydrogen, R² is G.113, X¹, X², X³, Y¹, Y², Y³, X⁴ and R⁵ have the values listed in the table P.

### Table 114 P

Table 114P provides 3240 compounds of formula (I-A) wherein G¹ is oxygen, R¹ is hydrogen, R² is G.114, X¹, X², X³, Y¹, Y², Y³, X⁴ and R⁵ have the values listed in the table P.

### Table 115 P

Table 115P provides 3240 compounds of formula (I-A) wherein G¹ is oxygen, R¹ is hydrogen, R² is G.115, X¹, X², X³, Y¹, Y², Y³, X⁴ and R⁵ have the values listed in the table P.

### Table 116 P

Table 116P provides 3240 compounds of formula (I-A) wherein G¹ is oxygen, R¹ is hydrogen, R² is G.116, X¹, X², X³, Y¹, Y², Y³, X⁴ and R⁵ have the values listed in the table P.

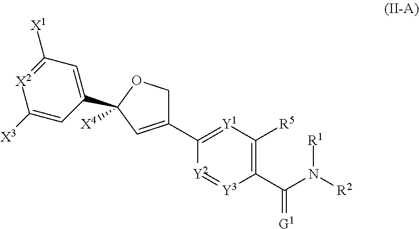

### Table 117 P

Table 117P provides 3240 compounds of formula (II-A) wherein G¹ is oxygen, R¹ is hydrogen, R² is G.001, X¹, X², X³, Y¹, Y², Y³, X⁴ and R⁵ have the values listed in the table P.

### Table 118 P

Table 118P provides 3240 compounds of formula (II-A) wherein G¹ is oxygen, R¹ is hydrogen, R² is G.002, X¹, X², X³, Y¹, Y², Y³, X⁴ and R⁵ have the values listed in the table P.

### Table 119 P

Table 119P provides 3240 compounds of formula (II-A) wherein G¹ is oxygen, R¹ is hydrogen, R² is G.003, X¹, X², X³, Y¹, Y², Y³, X⁴ and R⁵ have the values listed in the table P.

### Table 120 P

Table 120P provides 3240 compounds of formula (II-A) wherein G¹ is oxygen, R¹ is hydrogen, R² is G.004, X¹, X², X³, Y¹, Y², Y³, X⁴ and R⁵ have the values listed in the table P.

### Table 121 P

Table 121P provides 3240 compounds of formula (II-A) wherein G¹ is oxygen, R¹ is hydrogen, R² is G.005, X¹, X², X³, Y¹, Y², Y³, X⁴ and R⁵ have the values listed in the table P.

### Table 122 P

Table 122P provides 3240 compounds of formula (II-A) wherein G¹ is oxygen, R¹ is hydrogen, R² is G.006, X¹, X², X³, Y¹, Y², Y³, X⁴ and R⁵ have the values listed in the table P.

### Table 123 P

Table 123P provides 3240 compounds of formula (II-A) wherein G¹ is oxygen, R¹ is hydrogen, R² is G.007, X¹, X², X³, Y¹, Y², Y³, X⁴ and R⁵ have the values listed in the table P.

### Table 124 P

Table 124P provides 3240 compounds of formula (II-A) wherein G¹ is oxygen, R¹ is hydrogen, R² is G.008, X¹, X², X³, Y¹, Y², Y³, X⁴ and R⁵ have the values listed in the table P.

### Table 125 P

Table 125P provides 3240 compounds of formula (II-A) wherein G¹ is oxygen, R¹ is hydrogen, R² is G.009, X¹, X², X³, Y¹, Y², Y³, X⁴ and R⁵ have the values listed in the table P.

### Table 126 P

Table 126P provides 3240 compounds of formula (II-A) wherein G¹ is oxygen, R¹ is hydrogen, R² is G.010, X¹, X², X³, Y¹, Y², Y³, X⁴ and R⁵ have the values listed in the table P.

### Table 127 P

Table 127P provides 3240 compounds of formula (II-A) wherein G¹ is oxygen, R¹ is hydrogen, R² is G.011, X¹, X², X³, Y¹, Y², Y³, X⁴ and R⁵ have the values listed in the table P.

### Table 128 P

Table 128P provides 3240 compounds of formula (II-A) wherein G¹ is oxygen, R¹ is hydrogen, R² is G.012, X¹, X², X³, Y¹, Y², Y³, X⁴ and R⁵ have the values listed in the table P.

### Table 129 P

Table 129P provides 3240 compounds of formula (II-A) wherein G¹ is oxygen, R¹ is hydrogen, R² is G.013, X¹, X², X³, Y¹, Y², Y³, X⁴ and R⁵ have the values listed in the table P.

### Table 130 P

Table 130P provides 3240 compounds of formula (II-A) wherein G¹ is oxygen, R¹ is hydrogen, R² is G.014, X¹, X², X³, Y¹, Y², Y³, X⁴ and R⁵ have the values listed in the table P.

### Table 131 P

Table 131P provides 3240 compounds of formula (II-A) wherein G¹ is oxygen, R¹ is hydrogen, R² is G.015, X¹, X², X³, Y¹, Y², Y³, X⁴ and R⁵ have the values listed in the table P.

### Table 132 P

Table 132P provides 3240 compounds of formula (II-A) wherein G¹ is oxygen, R¹ is hydrogen, R² is G.016, X¹, X², X³, Y¹, Y², Y³, X⁴ and R⁵ have the values listed in the table P.

### Table 133 P

Table 133P provides 3240 compounds of formula (II-A) wherein G¹ is oxygen, R¹ is hydrogen, R² is G.017, X¹, X², X³, Y¹, Y², Y³, X⁴ and R⁵ have the values listed in the table P.

### Table 134 P

Table 134P provides 3240 compounds of formula (II-A) wherein G¹ is oxygen, R¹ is hydrogen, R² is G.018, X¹, X², X³, Y¹, Y², Y³, X⁴ and R⁵ have the values listed in the table P.

### Table 135 P

Table 135P provides 3240 compounds of formula (II-A) wherein G¹ is oxygen, R¹ is hydrogen, R² is G.019, X¹, X², X³, Y¹, Y², Y³, X⁴ and R⁵ have the values listed in the table P.

### Table 136 P

Table 136P provides 3240 compounds of formula (II-A) wherein G¹ is oxygen, R¹ is hydrogen, R² is G.020, X¹, X², X³, Y¹, Y², Y³, X⁴ and R⁵ have the values listed in the table P.

### Table 137 P

Table 137P provides 3240 compounds of formula (II-A) wherein G¹ is oxygen, R¹ is hydrogen, R² is G.021, X¹, X², X³, Y¹, Y², Y³, X⁴ and R⁵ have the values listed in the table P.

### Table 138 P

Table 138P provides 3240 compounds of formula (II-A) wherein G¹ is oxygen, R¹ is hydrogen, R² is G.022, X¹, X², X³, Y¹, Y², Y³, X⁴ and R⁵ have the values listed in the table P.

### Table 139 P

Table 139P provides 3240 compounds of formula (II-A) wherein G¹ is oxygen, R¹ is hydrogen, R² is G.023, X¹, X², X³, Y¹, Y², Y³, X⁴ and R⁵ have the values listed in the table P.

### Table 140 P

Table 140P provides 3240 compounds of formula (II-A) wherein G¹ is oxygen, R¹ is hydrogen, R² is G.024, X¹, X², X³, Y¹, Y², Y³, X⁴ and R⁵ have the values listed in the table P.

### Table 141 P

Table 141P provides 3240 compounds of formula (II-A) wherein G¹ is oxygen, R¹ is hydrogen, R² is G.025, X¹, X², X³, Y¹, Y², Y³, X⁴ and R⁵ have the values listed in the table P.

### Table 142 P

Table 142P provides 3240 compounds of formula (II-A) wherein G¹ is oxygen, R¹ is hydrogen, R² is G.026, X¹, X², X³, Y¹, Y², Y³, X⁴ and R⁵ have the values listed in the table P.

### Table 143 P

Table 143P provides 3240 compounds of formula (II-A) wherein G¹ is oxygen, R¹ is hydrogen, R² is G.027, X¹, X², X³, Y¹, Y², Y³, X⁴ and R⁵ have the values listed in the table P.

### Table 144 P

Table 144P provides 3240 compounds of formula (II-A) wherein G¹ is oxygen, R¹ is hydrogen, R² is G.028, X¹, X², X³, Y¹, Y², Y³, X⁴ and R⁵ have the values listed in the table P.

### Table 145 P

Table 145P provides 3240 compounds of formula (II-A) wherein G¹ is oxygen, R¹ is hydrogen, R² is G.029, X¹, X², X³, Y¹, Y², Y³, X⁴ and R⁵ have the values listed in the table P.

### Table 146 P

Table 146P provides 3240 compounds of formula (II-A) wherein G¹ is oxygen, R¹ is hydrogen, R² is G.030, X¹, X², X³, Y¹, Y², Y³, X⁴ and R⁵ have the values listed in the table P.

### Table 147 P

Table 147P provides 3240 compounds of formula (II-A) wherein G¹ is oxygen, R¹ is hydrogen, R² is G.031, X¹, X², X³, Y¹, Y², Y³, X⁴ and R⁵ have the values listed in the table P.

### Table 148 P

Table 148P provides 3240 compounds of formula (II-A) wherein G¹ is oxygen, R¹ is hydrogen, R² is G.032, X¹, X², X³, Y¹, Y², Y³, X⁴ and R⁵ have the values listed in the table P.

### Table 149 P

Table 149P provides 3240 compounds of formula (II-A) wherein G¹ is oxygen, R¹ is hydrogen, R² is G.033, X¹, X², X³, Y¹, Y², Y³, X⁴ and R⁵ have the values listed in the table P.

### Table 150 P

Table 150P provides 3240 compounds of formula (II-A) wherein G¹ is oxygen, R¹ is hydrogen, R² is G.034, X¹, X², X³, Y¹, Y², Y³, X⁴ and R⁵ have the values listed in the table P.

### Table 151 P

Table 151P provides 3240 compounds of formula (II-A) wherein G¹ is oxygen, R¹ is hydrogen, R² is G.035, X¹, X², X³, Y¹, Y², Y³, X⁴ and R⁵ have the values listed in the table P.

### Table 152 P

Table 152P provides 3240 compounds of formula (II-A) wherein G¹ is oxygen, R¹ is hydrogen, R² is G.036, X¹, X², X³, Y¹, Y², Y³, X⁴ and R⁵ have the values listed in the table P.

### Table 153 P

Table 153P provides 3240 compounds of formula (II-A) wherein G¹ is oxygen, R¹ is hydrogen, R² is G.037, X¹, X², X³, Y¹, Y², Y³, X⁴ and R⁵ have the values listed in the table P.

### Table 154 P

Table 154P provides 3240 compounds of formula (II-A) wherein G¹ is oxygen, R¹ is hydrogen, R² is G.038, X¹, X², X³, Y¹, Y², Y³, X⁴ and R⁵ have the values listed in the table P.

### Table 155 P

Table 155P provides 3240 compounds of formula (II-A) wherein G¹ is oxygen, R¹ is hydrogen, R² is G.039, X¹, X², X³, Y¹, Y², Y³, X⁴ and R⁵ have the values listed in the table P.

### Table 156 P

Table 156P provides 3240 compounds of formula (II-A) wherein G¹ is oxygen, R¹ is hydrogen, R² is G.040, X¹, X², X³, Y¹, Y², Y³, X⁴ and R⁵ have the values listed in the table P.

### Table 157 P

Table 157P provides 3240 compounds of formula (II-A) wherein G¹ is oxygen, R¹ is hydrogen, R² is G.041, X¹, X², X³, Y¹, Y², Y³, X⁴ and R⁵ have the values listed in the table P.

### Table 158 P

Table 158P provides 3240 compounds of formula (II-A) wherein G¹ is oxygen, R¹ is hydrogen, R² is G.042, X¹, X², X³, Y¹, Y², Y³, X⁴ and R⁵ have the values listed in the table P.

### Table 159 P

Table 159P provides 3240 compounds of formula (II-A) wherein G¹ is oxygen, R¹ is hydrogen, R² is G.043, X¹, X², X³, Y¹, Y², Y³, X⁴ and R⁵ have the values listed in the table P.

### Table 160 P

Table 160P provides 3240 compounds of formula (II-A) wherein G¹ is oxygen, R¹ is hydrogen, R² is G.044, X¹, X², X³, Y¹, Y², Y³, X⁴ and R⁵ have the values listed in the table P.

### Table 161 P

Table 161P provides 3240 compounds of formula (II-A) wherein G¹ is oxygen, R¹ is hydrogen, R² is G.045, X¹, X², X³, Y¹, Y², Y³, X⁴ and R⁵ have the values listed in the table P.

### Table 162 P

Table 162P provides 3240 compounds of formula (II-A) wherein G¹ is oxygen, R¹ is hydrogen, R² is G.046, X¹, X², X³, Y¹, Y², Y³, X⁴ and R⁵ have the values listed in the table P.

### Table 163 P

Table 163P provides 3240 compounds of formula (II-A) wherein G¹ is oxygen, R¹ is hydrogen, R² is G.047, X¹, X², X³, Y¹, Y², Y³, X⁴ and R⁵ have the values listed in the table P.

### Table 164 P

Table 164P provides 3240 compounds of formula (II-A) wherein G¹ is oxygen, R¹ is hydrogen, R² is G.048, X¹, X², X³, Y¹, Y², Y³, X⁴ and R⁵ have the values listed in the table P.

### Table 165 P

Table 165P provides 3240 compounds of formula (II-A) wherein G¹ is oxygen, R¹ is hydrogen, R² is G.049, X¹, X², X³, Y¹, Y², Y³, X⁴ and R⁵ have the values listed in the table P.

### Table 166 P

Table 166P provides 3240 compounds of formula (II-A) wherein G¹ is oxygen, R¹ is hydrogen, R² is G.050, X¹, X², X³, Y¹, Y², Y³, X⁴ and R⁵ have the values listed in the table P.

### Table 167 P

Table 167P provides 3240 compounds of formula (II-A) wherein G¹ is oxygen, R¹ is hydrogen, R² is G.051, X¹, X², X³, Y¹, Y², Y³, X⁴ and R⁵ have the values listed in the table P.

### Table 168 P

Table 168P provides 3240 compounds of formula (II-A) wherein G¹ is oxygen, R¹ is hydrogen, R² is G.052, X¹, X², X³, Y¹, Y², Y³, X⁴ and R⁵ have the values listed in the table P.

### Table 169 P

Table 169P provides 3240 compounds of formula (II-A) wherein G¹ is oxygen, R¹ is hydrogen, R² is G.053, X¹, X², X³, Y¹, Y², Y³, X⁴ and R⁵ have the values listed in the table P.

### Table 170 P

Table 170P provides 3240 compounds of formula (II-A) wherein G¹ is oxygen, R¹ is hydrogen, R² is G.054, X¹, X², X³, Y¹, Y², Y³, X⁴ and R⁵ have the values listed in the table P.

### Table 171 P

Table 171P provides 3240 compounds of formula (II-A) wherein G¹ is oxygen, R¹ is hydrogen, R² is G.055, X¹, X², X³, Y¹, Y², Y³, X⁴ and R⁵ have the values listed in the table P.

### Table 172 P

Table 172P provides 3240 compounds of formula (II-A) wherein G¹ is oxygen, R¹ is hydrogen, R² is G.056, X¹, X², X³, Y¹, Y², Y³, X⁴ and R⁵ have the values listed in the table P.

### Table 173 P

Table 173P provides 3240 compounds of formula (II-A) wherein G¹ is oxygen, R¹ is hydrogen, R² is G.057, X¹, X², X³, Y¹, Y², Y³, X⁴ and R⁵ have the values listed in the table P.

### Table 174 P

Table 174P provides 3240 compounds of formula (II-A) wherein G¹ is oxygen, R¹ is hydrogen, R² is G.058, X¹, X², X³, Y¹, Y², Y³, X⁴ and R⁵ have the values listed in the table P.

### Table 175 P

Table 175P provides 3240 compounds of formula (II-A) wherein G¹ is oxygen, R¹ is hydrogen, R² is G.059, X¹, X², X³, Y¹, Y², Y³, X⁴ and R⁵ have the values listed in the table P.

### Table 176 P

Table 176P provides 3240 compounds of formula (II-A) wherein G¹ is oxygen, R¹ is hydrogen, R² is G.060, X¹, X², X³, Y¹, Y², Y³, X⁴ and R⁵ have the values listed in the table P.

### Table 177 P

Table 177P provides 3240 compounds of formula (II-A) wherein G¹ is oxygen, R¹ is hydrogen, R² is G.061, X¹, X², X³, Y¹, Y², Y³, X⁴ and R⁵ have the values listed in the table P.

### Table 178 P

Table 178P provides 3240 compounds of formula (II-A) wherein G¹ is oxygen, R¹ is hydrogen, R² is G.062, X¹, X², X³, Y¹, Y², Y³, X⁴ and R⁵ have the values listed in the table P.

### Table 179 P

Table 179P provides 3240 compounds of formula (II-A) wherein G¹ is oxygen, R¹ is hydrogen, R² is G.063, X¹, X², X³, Y¹, Y², Y³, X⁴ and R⁵ have the values listed in the table P.

### Table 180 P

Table 180P provides 3240 compounds of formula (II-A) wherein G¹ is oxygen, R¹ is hydrogen, R² is G.064, X¹, X², X³, Y¹, Y², Y³, X⁴ and R⁵ have the values listed in the table P.

### Table 181 P

Table 181P provides 3240 compounds of formula (II-A) wherein G¹ is oxygen, R¹ is hydrogen, R² is G.065, X¹, X², X³, Y¹, Y², Y³, X⁴ and R⁵ have the values listed in the table P.

### Table 182 P

Table 182P provides 3240 compounds of formula (II-A) wherein G¹ is oxygen, R¹ is hydrogen, R² is G.066, X¹, X², X³, Y¹, Y², Y³, X⁴ and R⁵ have the values listed in the table P.

### Table 183 P

Table 183P provides 3240 compounds of formula (II-A) wherein G¹ is oxygen, R¹ is hydrogen, R² is G.067, X¹, X², X³, Y¹, Y², Y³, X⁴ and R⁵ have the values listed in the table P.

### Table 184 P

Table 184P provides 3240 compounds of formula (II-A) wherein G¹ is oxygen, R¹ is hydrogen, R² is G.068, X¹, X², X³, Y¹, Y², Y³, X⁴ and R⁵ have the values listed in the table P.

### Table 185 P

Table 185P provides 3240 compounds of formula (II-A) wherein G¹ is oxygen, R¹ is hydrogen, R² is G.069, X¹, X², X³, Y¹, Y², Y³, X⁴ and R⁵ have the values listed in the table P.

### Table 186 P

Table 186P provides 3240 compounds of formula (II-A) wherein G¹ is oxygen, R¹ is hydrogen, R² is G.070, X¹, X², X³, Y¹, Y², Y³, X⁴ and R⁵ have the values listed in the table P.

### Table 187 P

Table 187P provides 3240 compounds of formula (II-A) wherein G¹ is oxygen, R¹ is hydrogen, R² is G.071, X¹, X², X³, Y¹, Y², Y³, X⁴ and R⁵ have the values listed in the table P.

### Table 188 P

Table 188P provides 3240 compounds of formula (II-A) wherein G¹ is oxygen, R¹ is hydrogen, R² is G.072, X¹, X², X³, Y¹, Y², Y³, X⁴ and R⁵ have the values listed in the table P.

### Table 189 P

Table 189P provides 3240 compounds of formula (II-A) wherein G¹ is oxygen, R¹ is hydrogen, R² is G.073, X¹, X², X³, Y¹, Y², Y³, X⁴ and R⁵ have the values listed in the table P.

### Table 190 P

Table 190P provides 3240 compounds of formula (II-A) wherein G¹ is oxygen, R¹ is hydrogen, R² is G.074, X¹, X², X³, Y¹, Y², Y³, X⁴ and R⁵ have the values listed in the table P.

### Table 191 P

Table 191P provides 3240 compounds of formula (II-A) wherein G¹ is oxygen, R¹ is hydrogen, R² is G.075, X¹, X², X³, Y¹, Y², Y³, X⁴ and R⁵ have the values listed in the table P.

### Table 192 P

Table 192P provides 3240 compounds of formula (II-A) wherein G¹ is oxygen, R¹ is hydrogen, R² is G.076, X¹, X², X³, Y¹, Y², Y³, X⁴ and R⁵ have the values listed in the table P.

### Table 193 P

Table 193P provides 3240 compounds of formula (II-A) wherein G¹ is oxygen, R¹ is hydrogen, R² is G.077, X¹, X², X³, Y¹, Y², Y³, X⁴ and R⁵ have the values listed in the table P.

### Table 194 P

Table 194P provides 3240 compounds of formula (II-A) wherein G¹ is oxygen, R¹ is hydrogen, R² is G.078, X¹, X², X³, Y¹, Y², Y³, X⁴ and R⁵ have the values listed in the table P.

### Table 195 P

Table 195P provides 3240 compounds of formula (II-A) wherein G¹ is oxygen, R¹ is hydrogen, R² is G.079, X¹, X², X³, Y¹, Y², Y³, X⁴ and R⁵ have the values listed in the table P.

### Table 196 P

Table 196P provides 3240 compounds of formula (II-A) wherein G¹ is oxygen, R¹ is hydrogen, R² is G.080, X¹, X², X³, Y¹, Y², Y³, X⁴ and R⁵ have the values listed in the table P.

### Table 197 P

Table 197P provides 3240 compounds of formula (II-A) wherein G¹ is oxygen, R¹ is hydrogen, R² is G.081, X¹, X², X³, Y¹, Y², Y³, X⁴ and R⁵ have the values listed in the table P.

### Table 198 P

Table 198P provides 3240 compounds of formula (II-A) wherein G¹ is oxygen, R¹ is hydrogen, R² is G.082, X¹, X², X³, Y¹, Y², Y³, X⁴ and R⁵ have the values listed in the table P.

### Table 199 P

Table 199P provides 3240 compounds of formula (II-A) wherein G¹ is oxygen, R¹ is hydrogen, R² is G.083, X¹, X², X³, Y¹, Y², Y³, X⁴ and R⁵ have the values listed in the table P.

### Table 200 P

Table 200P provides 3240 compounds of formula (II-A) wherein G¹ is oxygen, R¹ is hydrogen, R² is G.084, X¹, X², X³, Y¹, Y², Y³, X⁴ and R⁵ have the values listed in the table P.

### Table 201 P

Table 201P provides 3240 compounds of formula (II-A) wherein G¹ is oxygen, R¹ is hydrogen, R² is G.085, X¹, X², X³, Y¹, Y², Y³, X⁴ and R⁵ have the values listed in the table P.

### Table 202 P

Table 202P provides 3240 compounds of formula (II-A) wherein G¹ is oxygen, R¹ is hydrogen, R² is G.086, X¹, X², X³, Y¹, Y², Y³, X⁴ and R⁵ have the values listed in the table P.

### Table 203 P

Table 203P provides 3240 compounds of formula (II-A) wherein G¹ is oxygen, R¹ is hydrogen R² is G.087, X¹, X², X³, Y¹, Y², Y³, X⁴ and R⁵ have the values listed in the table P.

### Table 204 P

Table 204P provides 3240 compounds of formula (II-A) wherein G¹ is oxygen, R¹ is hydrogen, R² is G.088, X¹, X², X³, Y¹, Y², Y³, X⁴ and R⁵ have the values listed in the table P.

### Table 205 P

Table 205P provides 3240 compounds of formula (II-A) wherein G¹ is oxygen, R¹ is hydrogen, R² is G.089, X¹, X², X³, Y¹, Y², Y³, X⁴ and R⁵ have the values listed in the table P.

### Table 206 P

Table 206P provides 3240 compounds of formula (II-A) wherein G¹ is oxygen, R¹ is hydrogen R² is G.090, X¹, X², X³, Y¹, Y², Y³, X⁴ and R⁵ have the values listed in the table P.

### Table 207 P

Table 207P provides 3240 compounds of formula (II-A) wherein G¹ is oxygen, R¹ is hydrogen, R² is G.091, X¹, X², X³, Y¹, Y², Y³, X⁴ and R⁵ have the values listed in the table P.

### Table 208 P

Table 208P provides 3240 compounds of formula (II-A) wherein G¹ is oxygen, R¹ is hydrogen, R² is G.092, X¹, X², X³, Y¹, Y², Y³, X⁴ and R⁵ have the values listed in the table P.

### Table 209 P

Table 209P provides 3240 compounds of formula (II-A) wherein G¹ is oxygen, R¹ is hydrogen, R² is G.093, X¹, X², X³, Y¹, Y², Y³, X⁴ and R⁵ have the values listed in the table P.

### Table 210 P

Table 210P provides 3240 compounds of formula (II-A) wherein G¹ is oxygen, R¹ is hydrogen, R² is G.094, X¹, X², X³, Y¹, Y², Y³, X⁴ and R⁵ have the values listed in the table P.

### Table 211 P

Table 211P provides 3240 compounds of formula (II-A) wherein G¹ is oxygen, R¹ is hydrogen, R² is G.095, X¹, X², X³, Y¹, Y², Y³, X⁴ and R⁵ have the values listed in the table P.

### Table 212 P

Table 212P provides 3240 compounds of formula (II-A) wherein G¹ is oxygen, R¹ is hydrogen, R² is G.096, X¹, X², X³, Y¹, Y², Y³, X⁴ and R⁵ have the values listed in the table P.

### Table 213 P

Table 213P provides 3240 compounds of formula (II-A) wherein G¹ is oxygen, R¹ is hydrogen, R² is G.097, X¹, X², X³, Y¹, Y², Y³, X⁴ and R⁵ have the values listed in the table P.

### Table 214 P

Table 214P provides 3240 compounds of formula (II-A) wherein G¹ is oxygen, R¹ is hydrogen, R² is G.098, X¹, X², X³, Y¹, Y², Y³, X⁴ and R⁵ have the values listed in the table P.

### Table 215 P

Table 215P provides 3240 compounds of formula (II-A) wherein G¹ is oxygen, R¹ is hydrogen, R² is G.099, X¹, X², X³, Y¹, Y², Y³, X⁴ and R⁵ have the values listed in the table P.

### Table 216 P

Table 216P provides 3240 compounds of formula (II-A) wherein G¹ is oxygen, R¹ is hydrogen, R² is G.100, X¹, X², X³, Y¹, Y², Y³, X⁴ and R⁵ have the values listed in the table P.

### Table 217 P

Table 217P provides 3240 compounds of formula (II-A) wherein G¹ is oxygen, R¹ is hydrogen, R² is G.101, X¹, X², X³, Y¹, Y², Y³, X⁴ and R⁵ have the values listed in the table P.

### Table 218 P

Table 218P provides 3240 compounds of formula (II-A) wherein G¹ is oxygen, R¹ is hydrogen, R² is G.102, X¹, X², X³, Y¹, Y², Y³, X⁴ and R⁵ have the values listed in the table P.

### Table 219 P

Table 219P provides 3240 compounds of formula (II-A) wherein G¹ is oxygen, R¹ is hydrogen, R² is G.103, X¹, X², X³, Y¹, Y², Y³, X⁴ and R⁵ have the values listed in the table P.

### Table 220 P

Table 220P provides 3240 compounds of formula (II-A) wherein G¹ is oxygen, R¹ is hydrogen, R² is G.104, X¹, X², X³, Y¹, Y², Y³, X⁴ and R⁵ have the values listed in the table P.

### Table 221 P

Table 221P provides 3240 compounds of formula (II-A) wherein G¹ is oxygen, R¹ is hydrogen, R² is G.105, X¹, X², X³, Y¹, Y², Y³, X⁴ and R⁵ have the values listed in the table P.

### Table 222 P

Table 222P provides 3240 compounds of formula (II-A) wherein G¹ is oxygen, R¹ is hydrogen, R² is G.106, X¹, X², X³, Y¹, Y², Y³, X⁴ and R⁵ have the values listed in the table P.

### Table 223 P

Table 223P provides 3240 compounds of formula (II-A) wherein G¹ is oxygen, R¹ is hydrogen, R² is G.107, X¹, X², X³, Y¹, Y², Y³, X⁴ and R⁵ have the values listed in the table P.

### Table 224 P

Table 224P provides 3240 compounds of formula (II-A) wherein G¹ is oxygen, R¹ is hydrogen, R² is G.108, X¹, X², X³, Y¹, Y², Y³, X⁴ and R⁵ have the values listed in the table P.

### Table 225 P

Table 225P provides 3240 compounds of formula (II-A) wherein G¹ is oxygen, R¹ is hydrogen, R² is G.109, X¹, X², X³, Y¹, Y², Y³, X⁴ and R⁵ have the values listed in the table P.

### Table 226 P

Table 226P provides 3240 compounds of formula (II-A) wherein G¹ is oxygen, R¹ is hydrogen, R² is G.110, X¹, X², X³, Y¹, Y², Y³, X⁴ and R⁵ have the values listed in the table P.

### Table 227 P

Table 227P provides 3240 compounds of formula (II-A) wherein G¹ is oxygen, R¹ is hydrogen, R² is G.111, X¹, X², X³, Y¹, Y², Y³, X⁴ and R⁵ have the values listed in the table P.

### Table 228 P

Table 228P provides 3240 compounds of formula (II-A) wherein G¹ is oxygen, R¹ is hydrogen, R² is G.112, X¹, X², X³, Y¹, Y², Y³, X⁴ and R⁵ have the values listed in the table P.

### Table 229 P

Table 229P provides 3240 compounds of formula (II-A) wherein G¹ is oxygen, R¹ is hydrogen, R² is G.113, X¹, X², X³, Y¹, Y², Y³, X⁴ and R⁵ have the values listed in the table P.

### Table 230 P

Table 230P provides 3240 compounds of formula (II-A) wherein G¹ is oxygen, R¹ is hydrogen, R² is G.114, X¹, X², X³, Y¹, Y², Y³, X⁴ and R⁵ have the values listed in the table P.

### Table 231 P

Table 231P provides 3240 compounds of formula (II-A) wherein G¹ is oxygen, R¹ is hydrogen, R² is G.115, X¹, X², X³, Y¹, Y², Y³, X⁴ and R⁵ have the values listed in the table P.

### Table 232 P

Table 232P provides 3240 compounds of formula (II-A) wherein G¹ is oxygen, R¹ is hydrogen, R² is G.116, X¹, X², X³, Y¹, Y², Y³, X⁴ and R⁵ have the values listed in the table P.

TABLE Q

| | X1, X2, X3 | X4 |
|---|---|---|
| Q.001 | X¹ is chloro, X² is CH, X³ is chloro | chlorodifluoromethyl |
| Q.002 | X¹ is chloro, X² is CH, X³ is chloro | difluoromethyl |
| Q.003 | X¹ is chloro, X² is CH, X³ is chloro | trifluoromethyl |
| Q.004 | X¹ is chloro, X² is C—F, X³ is hydrogen | chlorodifluoromethyl |
| Q.005 | X¹ is chloro, X² is C—F, X³ is hydrogen | difluoromethyl |
| Q.006 | X¹ is chloro, X² is C—F, X³ is hydrogen | trifluoromethyl |
| Q.007 | X¹ is fluoro, X² is C—Cl, X³ is hydrogen | chlorodifluoromethyl |
| Q.008 | X¹ is fluoro, X² is C—Cl, X³ is hydrogen | difluoromethyl |
| Q.009 | X¹ is fluoro, X² is C—Cl, X³ is hydrogen | trifluoromethyl |
| Q.010 | X¹ is chloro, X² is C—Cl, X³ is hydrogen | chlorodifluoromethyl |
| Q.011 | X¹ is chloro, X² is C—Cl, X³ is hydrogen | difluoromethyl |
| Q.012 | X¹ is chloro, X² is C—Cl, X³ is hydrogen | trifluoromethyl |
| Q.013 | X¹ is chloro, X² is C—Br, X³ is chloro | chlorodifluoromethyl |
| Q.014 | X¹ is chloro, X² is C—Br, X³ is chloro | difluoromethyl |
| Q.015 | X¹ is chloro, X² is C—Br, X³ is chloro | trifluoromethyl |
| Q.016 | X¹ is chloro, X² is C—F, X³ is chloro | chlorodifluoromethyl |
| Q.017 | X¹ is chloro, X² is C—F, X³ is chloro | difluoromethyl |
| Q.018 | X¹ is chloro, X² is C—F, X³ is chloro | trifluoromethyl |
| Q.019 | X¹ is chloro, X² is C—Cl, X³ is chloro | chlorodifluoromethyl |
| Q.020 | X¹ is chloro, X² is C—Cl, X³ is chloro | difluoromethyl |
| Q.021 | X¹ is chloro, X² is C—Cl, X³ is chloro | trifluoromethyl |
| Q.022 | X¹ is chloro, X² is C—I, X³ is chloro | chlorodifluoromethyl |
| Q.023 | X¹ is chloro, X² is C—I, X³ is chloro | difluoromethyl |
| Q.024 | X¹ is chloro, X² is C—I, X³ is chloro | trifluoromethyl |
| Q.025 | X¹ is fluoro, X² is C—F, X³ is fluoro | chlorodifluoromethyl |
| Q.026 | X¹ is fluoro, X² is C—F, X³ is fluoro | difluoromethyl |
| Q.027 | X¹ is fluoro, X² is C—F, X³ is fluoro | trifluoromethyl |
| Q.028 | X¹ is chloro, X² is CH, X³ is bromo | chlorodifluoromethyl |
| Q.029 | X¹ is chloro, X² is CH, X³ is bromo | difluoromethyl |
| Q.030 | X¹ is chloro, X² is CH, X³ is bromo | trifluoromethyl |
| Q.031 | X¹ is chloro, X² is CH, X³ is bromo | chlorodifluoromethyl |
| Q.032 | X¹ is chloro, X² is CH, X³ is bromo | difluoromethyl |
| Q.033 | X¹ is chloro, X² is CH, X³ is bromo | trifluoromethyl |
| Q.034 | X¹ is chloro, X² is CH, X³ is bromo | chlorodifluoromethyl |
| Q.035 | X¹ is chloro, X² is CH, X³ is bromo | difluoromethyl |
| Q.036 | X¹ is chloro, X² is CH, X³ is bromo | trifluoromethyl |
| Q.037 | X¹ is chloro, X² is C—Cl, X³ is | chlorodifluoromethyl |
| | trifluoromethyl | |
| Q.038 | X¹ is chloro, X² is C—Cl, X³ is | difluoromethyl |
| | trifluoromethyl | |
| Q.039 | X¹ is chloro, X² is C—Cl, X³ is | trifluoromethyl |
| | trifluoromethyl | |
| Q.040 | X¹ is trifluoromethyl, X² is CH, X³ is | chlorodifluoromethyl |
| | trifluoromethyl | |
| Q.041 | X¹ is trifluoromethyl, X² is CH, X³ is | difluoromethyl |
| | trifluoromethyl | |
| Q.042 | X¹ is trifluoromethyl, X² is CH, X³ is | trifluoromethyl |
| | trifluoromethyl | |
| Q.043 | X¹ is trifluoromethyl, X² is CH, X³ is | chlorodifluoromethyl |
| | trifluoromethyl | |
| Q.044 | X¹ is trifluoromethyl, X² is CH, X³ is | difluoromethyl |
| | trifluoromethyl | |
| Q.045 | X¹ is trifluoromethyl, X² is CH, X³ is | trifluoromethyl |
| | trifluoromethyl | |
| Q.046 | X¹ is trifluoromethyl, X² is CH, X³ is | chlorodifluoromethyl |
| | hydrogen | |
| Q.047 | X¹ is trifluoromethyl, X² is CH, X³ is | difluoromethyl |
| | hydrogen | |
| Q.048 | X¹ is trifluoromethyl, X² is CH, X³ is | trifluoromethyl |
| | hydrogen | |
| Q.049 | X¹ is chloro, X² is N, X³ is chloro | chlorodifluoromethyl |
| Q.050 | X¹ is chloro, X² is N, X³ is chloro | difluoromethyl |
| Q.051 | X¹ is chloro, X² is N, X³ is chloro | trifluoromethyl |
| Q.052 | X¹ is trifluoromethyl, X² is N, X³ is | chlorodifluoromethyl |
| | trifluoromethyl | |
| Q.053 | X¹ is trifluoromethyl, X² is N, X³ is | difluoromethyl |
| | trifluoromethyl | |
| Q.054 | X¹ is trifluoromethyl, X² is N, X³ is | trifluoromethyl |
| | trifluoromethyl | |

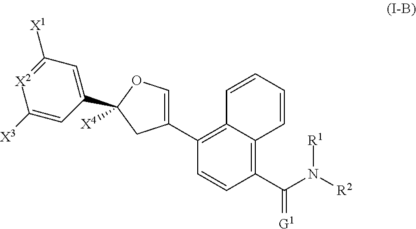

### Table 1Q:

Table 1Q provides 54 compounds of formula (I-B) wherein G¹ is oxygen, R¹ is hydrogen, R² is G.001, X¹, X², X³ and X4 have the values listed in the table Q.

### Table 2Q:

Table 2 Q provides 54 compounds of formula (I-B) wherein G¹ is oxygen, R¹ is hydrogen, R² is G.002, X¹, X², X³ and X4 have the values listed in the table Q.

### Table 3Q:

Table 3 Q provides 54 compounds of formula (I-B) wherein G¹ is oxygen, R¹ is hydrogen, R² is G.003, X¹, X², X³ and X4 have the values listed in the table Q.

### Table 4Q:

Table 4 Q provides 54 compounds of formula (I-B) wherein G¹ is oxygen, R¹ is hydrogen, R² is G.004, X¹, X², X³ and X4 have the values listed in the table Q.

### Table 5Q:

Table 5 Q provides 54 compounds of formula (I-B) wherein G¹ is oxygen, R¹ is hydrogen, R² is G.005, X¹, X², X³ and X4 have the values listed in the table Q.

### Table 6Q:

Table 6 Q provides 54 compounds of formula (I-B) wherein G¹ is oxygen, R¹ is hydrogen, R² is G.006, X¹, X², X³ and X4 have the values listed in the table Q.

### Table 7Q:

Table 7 Q provides 54 compounds of formula (I-B) wherein G¹ is oxygen, R¹ is hydrogen, R² is G.007, X¹, X², X³ and X4 have the values listed in the table Q.

### Table 8Q:

Table 8 Q provides 54 compounds of formula (I-B) wherein G¹ is oxygen, R¹ is hydrogen, R² is G.008, X¹, X², X³ and X4 have the values listed in the table Q.

### Table 9Q:

Table 9 Q provides 54 compounds of formula (I-B) wherein G¹ is oxygen, R¹ is hydrogen, R² is G.009, X¹, X², X³ and X4 have the values listed in the table Q.

### Table 10Q:

Table 10 Q provides 54 compounds of formula (I-B) wherein G¹ is oxygen, R¹ is hydrogen, R² is G.010, X¹, X², X³ and X4 have the values listed in the table Q.

### Table 11Q:

Table 11 Q provides 54 compounds of formula (I-B) wherein G¹ is oxygen, R¹ is hydrogen, R² is G.011, X¹, X², X³ and X4 have the values listed in the table Q.

### Table 12Q:

Table 12 Q provides 54 compounds of formula (I-B) wherein G¹ is oxygen, R¹ is hydrogen, R² is G.012, X¹, X², X³ and X4 have the values listed in the table Q.

### Table 13Q:

Table 13 Q provides 54 compounds of formula (I-B) wherein G¹ is oxygen, R¹ is hydrogen, R² is G.013, X¹, X², X³ and X4 have the values listed in the table Q.

### Table 14Q:

Table 14 Q provides 54 compounds of formula (I-B) wherein G¹ is oxygen, R¹ is hydrogen, R² is G.014, X¹, X², X³ and X4 have the values listed in the table Q.

### Table 15Q:

Table 15 Q provides 54 compounds of formula (I-B) wherein G¹ is oxygen, R¹ is hydrogen, R² is G.015, X¹, X², X³ and X4 have the values listed in the table Q.

### Table 16Q:

Table 16 Q provides 54 compounds of formula (I-B) wherein G¹ is oxygen, R¹ is hydrogen, R² is G.016, X¹, X², X³ and X4 have the values listed in the table Q.

### Table 17Q:

Table 17 Q provides 54 compounds of formula (I-B) wherein G¹ is oxygen, R¹ is hydrogen, R² is G.017, X¹, X², X³ and X4 have the values listed in the table Q.

### Table 18Q:

Table 18 Q provides 54 compounds of formula (I-B) wherein G¹ is oxygen, R¹ is hydrogen, R² is G.018, X¹, X², X³ and X4 have the values listed in the table Q.

### Table 19Q:

Table 19 Q provides 54 compounds of formula (I-B) wherein G¹ is oxygen, R¹ is hydrogen, R² is G.019, X¹, X², X³ and X4 have the values listed in the table Q.

### Table 20Q

Table 20 Q provides 54 compounds of formula (I-B) wherein G¹ is oxygen, R¹ is hydrogen, R² is G.020, X¹, X², X³ and X4 have the values listed in the table Q.

### Table 21Q:

Table 21 Q provides 54 compounds of formula (I-B) wherein G¹ is oxygen, R¹ is hydrogen, R² is G.021, X¹, X², X³ and X4 have the values listed in the table Q.

### Table 22Q:

Table 22 Q provides 54 compounds of formula (I-B) wherein G¹ is oxygen, R¹ is hydrogen, R² is G.022, X¹, X², X³ and X4 have the values listed in the table Q.

### Table 23Q:

Table 23 Q provides 54 compounds of formula (I-B) wherein G¹ is oxygen, R¹ is hydrogen, R² is G.023, X¹, X², X³ and X4 have the values listed in the table Q.

### Table 24Q:

Table 24 Q provides 54 compounds of formula (I-B) wherein G¹ is oxygen, R¹ is hydrogen, R² is G.024, X¹, X², X³ and X4 have the values listed in the table Q.

### Table 25Q:

Table 25 Q provides 54 compounds of formula (I-B) wherein G¹ is oxygen, R¹ is hydrogen, R² is G.025, X¹, X², X³ and X4 have the values listed in the table Q.

### Table 26Q:

Table 26 Q provides 54 compounds of formula (I-B) wherein G¹ is oxygen, R¹ is hydrogen, R² is G.026, X¹, X², X³ and X4 have the values listed in the table Q.

### Table 27Q:

Table 27 Q provides 54 compounds of formula (I-B) wherein G¹ is oxygen, R¹ is hydrogen, R² is G.027, X¹, X², X³ and X4 have the values listed in the table Q.

### Table 28Q:

Table 28 Q provides 54 compounds of formula (I-B) wherein G¹ is oxygen, R¹ is hydrogen, R² is G.028, X¹, X², X³ and X4 have the values listed in the table Q.

### Table 29Q:

Table 29 Q provides 54 compounds of formula (I-B) wherein G¹ is oxygen, R¹ is hydrogen, R² is G.029, X¹, X², X³ and X4 have the values listed in the table Q.

### Table 30Q:

Table 30 Q provides 54 compounds of formula (I-B) wherein G¹ is oxygen, R¹ is hydrogen, R² is G.030, X¹, X², X³ and X4 have the values listed in the table Q.

### Table 31Q:

Table 31 Q provides 54 compounds of formula (I-B) wherein G¹ is oxygen, R¹ is hydrogen, R² is G.031, X¹, X², X³ and X4 have the values listed in the table Q.

### Table 32Q:

Table 32 Q provides 54 compounds of formula (I-B) wherein G¹ is oxygen, R¹ is hydrogen, R² is G.032, X¹, X², X³ and X4 have the values listed in the table Q.

### Table 33Q:

Table 33 Q provides 54 compounds of formula (I-B) wherein G¹ is oxygen, R¹ is hydrogen, R² is G.033, X¹, X², X³ and X4 have the values listed in the table Q.

### Table 34Q:

Table 34 Q provides 54 compounds of formula (I-B) wherein G¹ is oxygen, R¹ is hydrogen, R² is G.034, X¹, X², X³ and X4 have the values listed in the table Q.

### Table 35Q:

Table 35 Q provides 54 compounds of formula (I-B) wherein G¹ is oxygen, R¹ is hydrogen, R² is G.035, X¹, X², X³ and X4 have the values listed in the table Q.

### Table 36Q:

Table 36 Q provides 54 compounds of formula (I-B) wherein G¹ is oxygen, R¹ is hydrogen, R² is G.036, X¹, X², X³ and X4 have the values listed in the table Q.

### Table 37Q:

Table 37 Q provides 54 compounds of formula (I-B) wherein G¹ is oxygen, R¹ is hydrogen, R² is G.037, X¹, X², X³ and X4 have the values listed in the table Q.

### Table 38Q:

Table 38 Q provides 54 compounds of formula (I-B) wherein G¹ is oxygen, R¹ is hydrogen, R² is G.038, X¹, X², X³ and X4 have the values listed in the table Q.

### Table 39Q:

Table 39 Q provides 54 compounds of formula (I-B) wherein G¹ is oxygen, R¹ is hydrogen, R² is G.039, X¹, X², X³ and X4 have the values listed in the table Q.

### Table 40Q:

Table 40 Q provides 54 compounds of formula (I-B) wherein G¹ is oxygen, R¹ is hydrogen, R² is G.040, X¹, X², X³ and X4 have the values listed in the table Q.

### Table 41Q:

Table 41 Q provides 54 compounds of formula (I-B) wherein G¹ is oxygen, R¹ is hydrogen, R² is G.041, X¹, X², X³ and X4 have the values listed in the table Q.

### Table 42Q:

Table 42 Q provides 54 compounds of formula (I-B) wherein G¹ is oxygen, R¹ is hydrogen, R² is G.042, X¹, X², X³ and X4 have the values listed in the table Q.

### Table 43Q:

Table 43 Q provides 54 compounds of formula (I-B) wherein G¹ is oxygen, R¹ is hydrogen, R² is G.043, X¹, X², X³ and X4 have the values listed in the table Q.

### Table 44Q:

Table 44 Q provides 54 compounds of formula (I-B) wherein G¹ is oxygen, R¹ is hydrogen, R² is G.044, X¹, X², X³ and X4 have the values listed in the table Q.

### Table 45Q:

Table 45 Q provides 54 compounds of formula (I-B) wherein G¹ is oxygen, R¹ is hydrogen, R² is G.045, X¹, X², X³ and X4 have the values listed in the table Q.

### Table 46Q:

Table 46 Q provides 54 compounds of formula (I-B) wherein G¹ is oxygen, R¹ is hydrogen, R² is G.046, X¹, X², X³ and X4 have the values listed in the table Q.

### Table 47Q:

Table 47 Q provides 54 compounds of formula (I-B) wherein G¹ is oxygen, R¹ is hydrogen, R² is G.047, X¹, X², X³ and X4 have the values listed in the table Q.

### Table 48Q:

Table 48 Q provides 54 compounds of formula (I-B) wherein G¹ is oxygen, R¹ is hydrogen, R² is G.048, X¹, X², X³ and X4 have the values listed in the table Q.

### Table 49Q:

Table 49 Q provides 54 compounds of formula (I-B) wherein G¹ is oxygen, R¹ is hydrogen, R² is G.049, X¹, X², X³ and X4 have the values listed in the table Q.

### Table 50Q:

Table 50 Q provides 54 compounds of formula (I-B) wherein G¹ is oxygen, R¹ is hydrogen, R² is G.050, X¹, X², X³ and X4 have the values listed in the table Q.

### Table 51Q:

Table 51 Q provides 54 compounds of formula (I-B) wherein G¹ is oxygen, R¹ is hydrogen, R² is G.051, X¹, X², X³ and X4 have the values listed in the table Q.

### Table 52Q:

Table 52 Q provides 54 compounds of formula (I-B) wherein G¹ is oxygen, R¹ is hydrogen, R² is G.052, X¹, X², X³ and X4 have the values listed in the table Q.

### Table 53Q:

Table 53 Q provides 54 compounds of formula (I-B) wherein G¹ is oxygen, R¹ is hydrogen, R² is G.053, X¹, X², X³ and X4 have the values listed in the table Q.

### Table 54Q:

Table 54 Q provides 54 compounds of formula (I-B) wherein G¹ is oxygen, R¹ is hydrogen, R² is G.054, X¹, X², X³ and X4 have the values listed in the table Q.

### Table 55Q:

Table 55 Q provides 54 compounds of formula (I-B) wherein G¹ is oxygen, R¹ is hydrogen, R² is G.055, X¹, X², X³ and X4 have the values listed in the table Q.

### Table 56Q:

Table 56 Q provides 54 compounds of formula (I-B) wherein G¹ is oxygen, R¹ is hydrogen, R² is G.056, X¹, X², X³ and X4 have the values listed in the table Q.

### Table 57Q:

Table 57 Q provides 54 compounds of formula (I-B) wherein G¹ is oxygen, R¹ is hydrogen, R² is G.057, X¹, X², X³ and X4 have the values listed in the table Q.

### Table 58Q:

Table 58 Q provides 54 compounds of formula (I-B) wherein G¹ is oxygen, R¹ is hydrogen, R² is G.058, X¹, X², X³ and X4 have the values listed in the table Q.

### Table 59Q:

Table 59 Q provides 54 compounds of formula (I-B) wherein G¹ is oxygen, R¹ is hydrogen, R² is G.059, X¹, X², X³ and X4 have the values listed in the table Q.

### Table 600:

Table 60 Q provides 54 compounds of formula (I-B) wherein G¹ is oxygen, R¹ is hydrogen, R² is G.060, X¹, X², X³ and X4 have the values listed in the table Q.

### Table 61Q:

Table 61 Q provides 54 compounds of formula (I-B) wherein G¹ is oxygen, R¹ is hydrogen, R² is G.061, X¹, X², X³ and X4 have the values listed in the table Q.

### Table 62Q:

Table 62 Q provides 54 compounds of formula (I-B) wherein G¹ is oxygen, R¹ is hydrogen, R² is G.062, X¹, X², X³ and X4 have the values listed in the table Q.

### Table 63Q:

Table 63 Q provides 54 compounds of formula (I-B) wherein G¹ is oxygen, R¹ is hydrogen, R² is G.063, X¹, X², X³ and X4 have the values listed in the table Q.

### Table 64Q:

Table 64 Q provides 54 compounds of formula (I-B) wherein G¹ is oxygen, R¹ is hydrogen, R² is G.064, X¹, X², X³ and X4 have the values listed in the table Q.

### Table 65Q:

Table 65 Q provides 54 compounds of formula (I-B) wherein G¹ is oxygen, R¹ is hydrogen, R² is G.065, X¹, X², X³ and X4 have the values listed in the table Q.

### Table 66Q:

Table 66 Q provides 54 compounds of formula (I-B) wherein G¹ is oxygen, R¹ is hydrogen, R² is G.066, X¹, X², X³ and X4 have the values listed in the table Q.

### Table 67Q:

Table 67 Q provides 54 compounds of formula (I-B) wherein G¹ is oxygen, R¹ is hydrogen, R² is G.067, X¹, X², X³ and X4 have the values listed in the table Q.

### Table 68Q:

Table 68 Q provides 54 compounds of formula (I-B) wherein G¹ is oxygen, R¹ is hydrogen, R² is G.068, X¹, X², X³ and X4 have the values listed in the table Q.

### Table 69Q:

Table 69 Q provides 54 compounds of formula (I-B) wherein G¹ is oxygen, R¹ is hydrogen, R² is G.069, X¹, X², X³ and X4 have the values listed in the table Q.

### Table 70Q:

Table 70 Q provides 54 compounds of formula (I-B) wherein G¹ is oxygen, R¹ is hydrogen, R² is G.070, X¹, X², X³ and X4 have the values listed in the table Q.

### Table 71Q:

Table 71 Q provides 54 compounds of formula (I-B) wherein G¹ is oxygen, R¹ is hydrogen, R² is G.071, X¹, X², X³ and X4 have the values listed in the table Q.

### Table 72Q:

Table 72 Q provides 54 compounds of formula (I-B) wherein G¹ is oxygen, R¹ is hydrogen, R² is G.072, X¹, X², X³ and X4 have the values listed in the table Q.

### Table 73Q:

Table 73 Q provides 54 compounds of formula (I-B) wherein G¹ is oxygen, R¹ is hydrogen, R² is G.073, X¹, X², X³ and X4 have the values listed in the table Q.

### Table 74Q:

Table 74 Q provides 54 compounds of formula (I-B) wherein G¹ is oxygen, R¹ is hydrogen, R² is G.074, X¹, X², X³ and X4 have the values listed in the table Q.

### Table 75Q:

Table 75 Q provides 54 compounds of formula (I-B) wherein G¹ is oxygen, R¹ is hydrogen, R² is G.075, X¹, X², X³ and X4 have the values listed in the table Q.

### Table 76Q:

Table 76 Q provides 54 compounds of formula (I-B) wherein G¹ is oxygen, R¹ is hydrogen, R² is G.076, X¹, X², X³ and X4 have the values listed in the table Q.

### Table 77Q:

Table 77 Q provides 54 compounds of formula (I-B) wherein G¹ is oxygen, R¹ is hydrogen, R² is G.077, X¹, X², X³ and X4 have the values listed in the table Q.

### Table 78Q:

Table 78 Q provides 54 compounds of formula (I-B) wherein G¹ is oxygen, R¹ is hydrogen, R² is G.078, X¹, X², X³ and X4 have the values listed in the table Q.

### Table 79Q:

Table 79 Q provides 54 compounds of formula (I-B) wherein G¹ is oxygen, R¹ is hydrogen, R² is G.079, X¹, X², X³ and X4 have the values listed in the table Q.

### Table 80Q:

Table 80 Q provides 54 compounds of formula (I-B) wherein G¹ is oxygen, R¹ is hydrogen, R² is G.080, X¹, X², X³ and X4 have the values listed in the table Q.

### Table 81Q:

Table 81 Q provides 54 compounds of formula (I-B) wherein G¹ is oxygen, R¹ is hydrogen, R² is G.081, X¹, X², X³ and X4 have the values listed in the table Q.

### Table 82Q:

Table 82 Q provides 54 compounds of formula (I-B) wherein G¹ is oxygen, R¹ is hydrogen, R² is G.082, X¹, X², X³ and X4 have the values listed in the table Q.

### Table 83Q:

Table 83 Q provides 54 compounds of formula (I-B) wherein G¹ is oxygen, R¹ is hydrogen, R² is G.083, X¹, X², X³ and X4 have the values listed in the table Q.

### Table 84Q:

Table 84 Q provides 54 compounds of formula (I-B) wherein G¹ is oxygen, R¹ is hydrogen, R² is G.084, X¹, X², X³ and X4 have the values listed in the table Q.

### Table 85Q:

Table 85 Q provides 54 compounds of formula (I-B) wherein G¹ is oxygen, R¹ is hydrogen, R² is G.085, X¹, X², X³ and X4 have the values listed in the table Q.

### Table 86Q:

Table 86 Q provides 54 compounds of formula (I-B) wherein G¹ is oxygen, R¹ is hydrogen, R² is G.086, X¹, X², X³ and X4 have the values listed in the table Q.

### Table 87Q:

Table 87 Q provides 54 compounds of formula (I-B) wherein G¹ is oxygen, R¹ is hydrogen, R² is G.087, X¹, X², X³ and X4 have the values listed in the table Q.

### Table 88Q:

Table 88 Q provides 54 compounds of formula (I-B) wherein G¹ is oxygen, R¹ is hydrogen, R² is G.088, X¹, X², X³ and X4 have the values listed in the table Q.

### Table 89Q:

Table 89 Q provides 54 compounds of formula (I-B) wherein G¹ is oxygen, R¹ is hydrogen, R² is G.089, X¹, X², X³ and X4 have the values listed in the table Q.

### Table 90Q:

Table 90 Q provides 54 compounds of formula (I-B) wherein G¹ is oxygen, R¹ is hydrogen, R² is G.090, X¹, X², X³ and X4 have the values listed in the table Q.

### Table 91Q:

Table 91 Q provides 54 compounds of formula (I-B) wherein G¹ is oxygen, R¹ is hydrogen, R² is G.091, X¹, X², X³ and X4 have the values listed in the table Q.

### Table 92 Q:

Table 92 Q provides 54 compounds of formula (I-B) wherein G¹ is oxygen, R¹ is hydrogen, R² is G.092, X¹, X², X³ and X4 have the values listed in the table Q.

### Table 93 Q:

Table 93 Q provides 54 compounds of formula (I-B) wherein G¹ is oxygen, R¹ is hydrogen, R² is G.093, X¹, X², X³ and X4 have the values listed in the table Q.

### Table 94

Table 94 Q provides 54 compounds of formula (I-B) wherein G¹ is oxygen, R¹ is hydrogen, R² is G.094, X¹, X², X³ and X4 have the values listed in the table Q.

### Table 95 Q:

Table 95 Q provides 54 compounds of formula (I-B) wherein G¹ is oxygen, R¹ is hydrogen, R² is G.095, X¹, X², X³ and X4 have the values listed in the table Q.

### Table 96 Q:

Table 96 Q provides 54 compounds of formula (I-B) wherein G¹ is oxygen, R¹ is hydrogen, R² is G.096, X¹, X², X³ and X4 have the values listed in the table Q.

### Table 97 Q:

Table 97 Q provides 54 compounds of formula (I-B) wherein G¹ is oxygen, R¹ is hydrogen, R² is G.097, X¹, X², X³ and X4 have the values listed in the table Q.

### Table 98 Q:

Table 98 Q provides 54 compounds of formula (I-B) wherein G¹ is oxygen, R¹ is hydrogen, R² is G.098, X¹, X², X³ and X4 have the values listed in the table Q.

### Table 99 Q:

Table 99 Q provides 54 compounds of formula (I-B) wherein G¹ is oxygen, R¹ is hydrogen, R² is G.099, X¹, X², X³ and X4 have the values listed in the table Q.

### Table 100 Q:

Table 100 Q provides 54 compounds of formula (I-B) wherein G¹ is oxygen, R¹ is hydrogen, R² is G.100, X¹, X², X³ and X4 have the values listed in the table Q.

### Table 101 Q:

Table 101 Q provides 54 compounds of formula (I-B) wherein G¹ is oxygen, R¹ is hydrogen, R² is G.101, X¹, X², X³ and X4 have the values listed in the table Q.

### Table 102 Q:

Table 102 Q provides 54 compounds of formula (I-B) wherein G¹ is oxygen, R¹ is hydrogen, R² is G.102, X¹, X², X³ and X4 have the values listed in the table Q.

### Table 103 Q:

Table 103 Q provides 54 compounds of formula (I-B) wherein G¹ is oxygen, R¹ is hydrogen, R² is G.103, X¹, X², X³ and X4 have the values listed in the table Q.

### Table 104 Q:

Table 104 Q provides 54 compounds of formula (I-B) wherein G¹ is oxygen, R¹ is hydrogen, R² is G.104, X¹, X², X³ and X4 have the values listed in the table Q.

### Table 105 Q:

Table 105 Q provides 54 compounds of formula (I-B) wherein G¹ is oxygen, R¹ is hydrogen, R² is G.105, X¹, X², X³ and X4 have the values listed in the table Q.

### Table 106 Q:

Table 106 Q provides 54 compounds of formula (I-B) wherein G¹ is oxygen, R¹ is hydrogen, R² is G.106, X¹, X², X³ and X4 have the values listed in the table Q.

### Table 107 Q:

Table 107 Q provides 54 compounds of formula (I-B) wherein G¹ is oxygen, R¹ is hydrogen, R² is G.107, X¹, X², X³ and X4 have the values listed in the table Q.

### Table 108 Q:

Table 108 Q provides 54 compounds of formula (I-B) wherein G¹ is oxygen, R¹ is hydrogen, R² is G.108, X¹, X², X³ and X4 have the values listed in the table Q.

### Table 109 Q:

Table 109 Q provides 54 compounds of formula (I-B) wherein G¹ is oxygen, R¹ is hydrogen, R² is G.109, X¹, X², X³ and X4 have the values listed in the table Q.

### Table 110 Q:

Table 110 Q provides 54 compounds of formula (I-B) wherein G¹ is oxygen, R¹ is hydrogen, R² is G.110, X¹, X², X³ and X4 have the values listed in the table Q.

### Table 111Q:

Table 111 Q provides 54 compounds of formula (I-B) wherein G¹ is oxygen, R¹ is hydrogen, R² is G.111, X¹, X², X³ and X4 have the values listed in the table Q.

### Table 112Q

Table 112 Q provides 54 compounds of formula (I-B) wherein G¹ is oxygen, R¹ is hydrogen, R² is G.112, X¹, X², X³ and X4 have the values listed in the table Q.

### Table 113 Q:

Table 113 Q provides 54 compounds of formula (I-B) wherein G¹ is oxygen, R¹ is hydrogen, R² is G.113, X¹, X², X³ and X4 have the values listed in the table Q.

### Table 114 Q:

Table 114 Q provides 54 compounds of formula (I-B) wherein G¹ is oxygen, R¹ is hydrogen, R² is G.114, X¹, X², X³ and X4 have the values listed in the table Q.

### Table 115 Q:

Table 115 Q provides 54 compounds of formula (I-B) wherein G¹ is oxygen, R¹ is hydrogen, R² is G.115, X¹, X², X³ and X4 have the values listed in the table Q.

### Table 116 Q:

Table 116 Q provides 54 compounds of formula (I-B) wherein G¹ is oxygen, R¹ is hydrogen, R² is G.116, X¹, X², X³ and X4 have the values listed in the table 0.

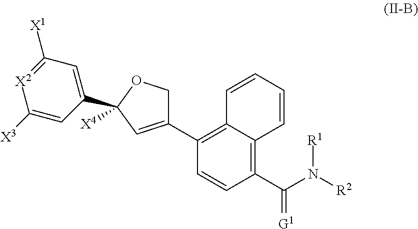

### Table 117 Q:

Table 117 Q provides 54 compounds of formula (II-B) wherein G¹ is oxygen, R¹ is hydrogen, R² is G.001, X¹, X², X³ and X4 have the values listed in the table Q.

### Table 118 Q:

Table 118 Q provides 54 compounds of formula (II-B) wherein G¹ is oxygen, R¹ is hydrogen, R² is G.002, X¹, X², X³ and X4 have the values listed in the table Q.

### Table 119 Q:

Table 119 Q provides 54 compounds of formula (II-B) wherein G¹ is oxygen, R¹ is hydrogen, R² is G.003, X¹, X², X³ and X4 have the values listed in the table Q.

### Table 120 Q:

Table 120 Q provides 54 compounds of formula (II-B) wherein G¹ is oxygen, R¹ is hydrogen, R² is G.004, X¹, X², X³ and X4 have the values listed in the table Q.

### Table 121 Q:

Table 121 Q provides 54 compounds of formula (II-B) wherein G¹ is oxygen, R¹ is hydrogen, R² is G.005, X¹, X², X³ and X4 have the values listed in the table Q.

### Table 122 Q:

Table 122 Q provides 54 compounds of formula (II-B) wherein G¹ is oxygen, R¹ is hydrogen, R² is G.006, X¹, X², X³ and X4 have the values listed in the table Q.

### Table 123 Q:

Table 123 Q provides 54 compounds of formula (II-B) wherein G¹ is oxygen, R¹ is hydrogen, R² is G.007, X¹, X², X³ and X4 have the values listed in the table Q.

### Table 124 Q:

Table 124 Q provides 54 compounds of formula (II-B) wherein G¹ is oxygen, R¹ is hydrogen, R² is G.008, X¹, X², X³ and X4 have the values listed in the table Q.

### Table 125 Q:

Table 125 Q provides 54 compounds of formula (II-B) wherein G¹ is oxygen, R¹ is hydrogen, R² is G.009, X¹, X², X³ and X4 have the values listed in the table Q.

### Table 126 Q:

Table 126 Q provides 54 compounds of formula (II-B) wherein G¹ is oxygen, R¹ is hydrogen, R² is G.010, X¹, X², X³ and X4 have the values listed in the table Q.

### Table 127 Q:

Table 127 Q provides 54 compounds of formula (II-B) wherein G¹ is oxygen, R¹ is hydrogen, R² is G.011, X¹, X², X³ and X4 have the values listed in the table Q.

### Table 128 Q:

Table 128 Q provides 54 compounds of formula (II-B) wherein G¹ is oxygen, R¹ is hydrogen, R² is G.012, X¹, X², X³ and X4 have the values listed in the table Q.

### Table 129 Q:

Table 129 Q provides 54 compounds of formula (II-B) wherein G¹ is oxygen, R¹ is hydrogen, R² is G.013, X¹, X², X³ and X4 have the values listed in the table Q.

### Table 130 Q:

Table 130 Q provides 54 compounds of formula (II-B) wherein G¹ is oxygen, R¹ is hydrogen, R² is G.014, X¹, X², X³ and X4 have the values listed in the table Q.

### Table 131 Q:

Table 131 Q provides 54 compounds of formula (II-B) wherein G¹ is oxygen, R¹ is hydrogen, R² is G.015, X¹, X², X³ and X4 have the values listed in the table Q.

### Table 132 Q:

Table 132 Q provides 54 compounds of formula (II-B) wherein G¹ is oxygen, R¹ is hydrogen, R² is G.016, X¹, X², X³ and X4 have the values listed in the table Q.

### Table 133 Q:

Table 133 Q provides 54 compounds of formula (II-B) wherein G¹ is oxygen, R¹ is hydrogen, R² is G.017, X¹, X², X³ and X4 have the values listed in the table Q.

### Table 134 Q:

Table 134 Q provides 54 compounds of formula (II-B) wherein G¹ is oxygen, R¹ is hydrogen, R² is G.018, X¹, X², X³ and X4 have the values listed in the table Q.

### Table 135 Q:

Table 135 Q provides 54 compounds of formula (II-B) wherein G¹ is oxygen, R¹ is hydrogen, R² is G.019, X¹, X², X³ and X4 have the values listed in the table Q.

### Table 136 Q:

Table 136 Q provides 54 compounds of formula (II-B) wherein G¹ is oxygen, R¹ is hydrogen, R² is G.020, X¹, X², X³ and X4 have the values listed in the table Q.

### Table 137 Q:

Table 137 Q provides 54 compounds of formula (II-B) wherein G¹ is oxygen, R¹ is hydrogen, R² is G.021, X¹, X², X³ and X4 have the values listed in the table Q.

### Table 138 Q:

Table 138 Q provides 54 compounds of formula (II-B) wherein G¹ is oxygen, R¹ is hydrogen, R² is G.022, X¹, X², X³ and X4 have the values listed in the table Q.

### Table 139 Q:

Table 139 Q provides 54 compounds of formula (II-B) wherein G¹ is oxygen, R¹ is hydrogen, R² is G.023, X¹, X², X³ and X4 have the values listed in the table Q.

### Table 140 Q:

Table 140 Q provides 54 compounds of formula (II-B) wherein G¹ is oxygen, R¹ is hydrogen, R² is G.024, X¹, X², X³ and X4 have the values listed in the table Q.

### Table 141 Q:

Table 141 Q provides 54 compounds of formula (II-B) wherein G¹ is oxygen, R¹ is hydrogen, R² is G.025, X¹, X², X³ and X4 have the values listed in the table Q.

### Table 142 Q:

Table 142 Q provides 54 compounds of formula (II-B) wherein G¹ is oxygen, R¹ is hydrogen, R² is G.026, X¹, X², X³ and X4 have the values listed in the table Q.

### Table 143 Q:

Table 143 Q provides 54 compounds of formula (II-B) wherein G¹ is oxygen, R¹ is hydrogen, R² is G.027, X¹, X², X³ and X4 have the values listed in the table Q.

### Table 144 Q:

Table 144 Q provides 54 compounds of formula (II-B) wherein G¹ is oxygen, R¹ is hydrogen, R² is G.028, X¹, X², X³ and X4 have the values listed in the table Q.

### Table 145 Q:

Table 145 Q provides 54 compounds of formula (II-B) wherein G¹ is oxygen, R¹ is hydrogen, R² is G.029, X¹, X², X³ and X4 have the values listed in the table Q.

### Table 146 Q:

Table 146 Q provides 54 compounds of formula (II-B) wherein G¹ is oxygen, R¹ is hydrogen, R² is G.030, X¹, X², X³ and X4 have the values listed in the table Q.

### Table 147 Q:

Table 147 Q provides 54 compounds of formula (II-B) wherein G¹ is oxygen, R¹ is hydrogen, R² is G.031, X¹, X², X³ and X4 have the values listed in the table Q.

### Table 148 Q:

Table 148 Q provides 54 compounds of formula (II-B) wherein G¹ is oxygen, R¹ is hydrogen, R² is G.032, X¹, X², X³ and X4 have the values listed in the table Q.

### Table 149 Q:

Table 149 Q provides 54 compounds of formula (II-B) wherein G¹ is oxygen, R¹ is hydrogen, R² is G.033, X¹, X², X³ and X4 have the values listed in the table Q.

### Table 150 Q:

Table 150 Q provides 54 compounds of formula (II-B) wherein G¹ is oxygen, R¹ is hydrogen, R² is G.034, X¹, X², X³ and X4 have the values listed in the table Q.

### Table 151 Q:

Table 151 Q provides 54 compounds of formula (II-B) wherein G¹ is oxygen, R¹ is hydrogen, R² is G.035, X¹, X², X³ and X4 have the values listed in the table Q.

### Table 152 Q:

Table 152 Q provides 54 compounds of formula (II-B) wherein G¹ is oxygen, R¹ is hydrogen, R² is G.036, X¹, X², X³ and X4 have the values listed in the table Q.

### Table 153 Q:

Table 153 Q provides 54 compounds of formula (II-B) wherein G¹ is oxygen, R¹ is hydrogen, R² is G.037, X¹, X², X³ and X4 have the values listed in the table Q.

### Table 154 Q:

Table 154 Q provides 54 compounds of formula (II-B) wherein G¹ is oxygen, R¹ is hydrogen, R² is G.038, X¹, X², X³ and X4 have the values listed in the table Q.

### Table 155 Q:

Table 155 Q provides 54 compounds of formula (II-B) wherein G¹ is oxygen, R¹ is hydrogen, R² is G.039, X¹, X², X³ and X4 have the values listed in the table Q.

### Table 156 Q:

Table 156 Q provides 54 compounds of formula (II-B) wherein G¹ is oxygen, R¹ is hydrogen, R² is G.040, X¹, X², X³ and X4 have the values listed in the table Q.

### Table 157 Q:

Table 157 Q provides 54 compounds of formula (II-B) wherein G¹ is oxygen, R¹ is hydrogen, R² is G.041, X¹, X², X³ and X4 have the values listed in the table Q.

### Table 158 Q:

Table 158 Q provides 54 compounds of formula (II-B) wherein G¹ is oxygen, R¹ is hydrogen, R² is G.042, X¹, X², X³ and X4 have the values listed in the table Q.

### Table 159 Q:

Table 159 Q provides 54 compounds of formula (II-B) wherein G¹ is oxygen, R¹ is hydrogen, R² is G.043, X¹, X², X³ and X4 have the values listed in the table Q.

### Table 160 Q:

Table 160 Q provides 54 compounds of formula (II-B) wherein G¹ is oxygen, R¹ is hydrogen, R² is G.044, X¹, X², X³ and X4 have the values listed in the table Q.

### Table 161 Q:

Table 161 Q provides 54 compounds of formula (II-B) wherein G¹ is oxygen, R¹ is hydrogen, R² is G.045, X¹, X², X³ and X4 have the values listed in the table Q.

### Table 162 Q:

Table 162 Q provides 54 compounds of formula (II-B) wherein G¹ is oxygen, R¹ is hydrogen, R² is G.046, X¹, X², X³ and X4 have the values listed in the table Q.

### Table 163 Q:

Table 163 Q provides 54 compounds of formula (II-B) wherein G¹ is oxygen, R¹ is hydrogen, R² is G.047, X¹, X², X³ and X4 have the values listed in the table Q.

### Table 164 Q:

Table 164 Q provides 54 compounds of formula (II-B) wherein G¹ is oxygen, R¹ is hydrogen, R² is G.048, X¹, X², X³ and X4 have the values listed in the table Q.

### Table 165 Q:

Table 165 Q provides 54 compounds of formula (II-B) wherein G¹ is oxygen, R¹ is hydrogen, R² is G.049, X¹, X², X³ and X4 have the values listed in the table Q.

### Table 166 Q:

Table 166 Q provides 54 compounds of formula (II-B) wherein G¹ is oxygen, R¹ is hydrogen, R² is G.050, X¹, X², X³ and X4 have the values listed in the table Q.

### Table 167 Q:

Table 167 Q provides 54 compounds of formula (II-B) wherein G¹ is oxygen, R¹ is hydrogen, R² is G.051, X¹, X², X³ and X4 have the values listed in the table Q.

### Table 168 Q:

Table 168 Q provides 54 compounds of formula (II-B) wherein G¹ is oxygen, R¹ is hydrogen, R² is G.052, X¹, X², X³ and X4 have the values listed in the table Q.

### Table 169 Q:

Table 169 Q provides 54 compounds of formula (II-B) wherein G¹ is oxygen, R¹ is hydrogen, R² is G.053, X¹, X², X³ and X4 have the values listed in the table Q.

### Table 170 Q:

Table 170 Q provides 54 compounds of formula (II-B) wherein G¹ is oxygen, R¹ is hydrogen, R² is G.054, X¹, X², X³ and X4 have the values listed in the table Q.

### Table 171 Q:

Table 171 Q provides 54 compounds of formula (II-B) wherein G¹ is oxygen, R¹ is hydrogen, R² is G.055, X¹, X², X³ and X4 have the values listed in the table Q.

### Table 172 Q:

Table 172 Q provides 54 compounds of formula (II-B) wherein G¹ is oxygen, R¹ is hydrogen, R² is G.056, X¹, X², X³ and X4 have the values listed in the table Q.

### Table 173 Q:

Table 173 Q provides 54 compounds of formula (II-B) wherein G¹ is oxygen, R¹ is hydrogen, R² is G.057, X¹, X², X³ and X4 have the values listed in the table Q.

### Table 174 Q:

Table 174 Q provides 54 compounds of formula (II-B) wherein G¹ is oxygen, R¹ is hydrogen, R² is G.058, X¹, X², X³ and X4 have the values listed in the table Q.

### Table 175 Q:

Table 175 Q provides 54 compounds of formula (II-B) wherein G¹ is oxygen, R¹ is hydrogen, R² is G.059, X¹, X², X³ and X4 have the values listed in the table Q.

### Table 176 Q:

Table 176 Q provides 54 compounds of formula (II-B) wherein G¹ is oxygen, R¹ is hydrogen, R² is G.060, X¹, X², X³ and X4 have the values listed in the table Q.

### Table 177 Q:

Table 177 Q provides 54 compounds of formula (II-B) wherein G¹ is oxygen, R¹ is hydrogen, R² is G.061, X¹, X², X³ and X4 have the values listed in the table Q.

### Table 178 Q:

Table 178 Q provides 54 compounds of formula (II-B) wherein G¹ is oxygen, R¹ is hydrogen, R² is G.062, X¹, X², X³ and X4 have the values listed in the table Q.

### Table 179 Q:

Table 179 Q provides 54 compounds of formula (II-B) wherein G¹ is oxygen, R¹ is hydrogen, R² is G.063, X¹, X², X³ and X4 have the values listed in the table Q.

### Table 180 Q:

Table 180 Q provides 54 compounds of formula (II-B) wherein G¹ is oxygen, R¹ is hydrogen, R² is G.064, X¹, X², X³ and X4 have the values listed in the table Q.

### Table 181 Q:

Table 181 Q provides 54 compounds of formula (II-B) wherein G¹ is oxygen, R¹ is hydrogen, R² is G.065, X¹, X², X³ and X4 have the values listed in the table Q.

### Table 182 Q:

Table 182 Q provides 54 compounds of formula (II-B) wherein G¹ is oxygen, R¹ is hydrogen, R² is G.066, X¹, X², X³ and X4 have the values listed in the table Q.

### Table 183 Q:

Table 183 Q provides 54 compounds of formula (II-B) wherein G¹ is oxygen, R¹ is hydrogen, R² is G.067, X¹, X², X³ and X4 have the values listed in the table Q.

### Table 184 Q:

Table 184 Q provides 54 compounds of formula (II-B) wherein G¹ is oxygen, R¹ is hydrogen, R² is G.068, X¹, X², X³ and X4 have the values listed in the table Q.

### Table 185 Q:

Table 185 Q provides 54 compounds of formula (II-B) wherein G¹ is oxygen, R¹ is hydrogen, R² is G.069, X¹, X², X³ and X4 have the values listed in the table Q.

### Table 186 Q:

Table 186 Q provides 54 compounds of formula (II-B) wherein G¹ is oxygen, R¹ is hydrogen, R² is G.070, X¹, X², X³ and X4 have the values listed in the table Q.

### Table 187 Q:

Table 187 Q provides 54 compounds of formula (II-B) wherein G¹ is oxygen, R¹ is hydrogen, R² is G.071, X¹, X², X³ and X4 have the values listed in the table Q.

### Table 188 Q:

Table 188 Q provides 54 compounds of formula (II-B) wherein G¹ is oxygen, R¹ is hydrogen, R² is G.072, X¹, X², X³ and X4 have the values listed in the table Q.

### Table 189 Q:

Table 189 Q provides 54 compounds of formula (II-B) wherein G¹ is oxygen, R¹ is hydrogen, R² is G.073, X¹, X², X³ and X4 have the values listed in the table Q.

### Table 190 Q:

Table 190 Q provides 54 compounds of formula (II-B) wherein G¹ is oxygen, R¹ is hydrogen, R² is G.074, X¹, X², X³ and X4 have the values listed in the table Q.

### Table 191 Q:

Table 191 Q provides 54 compounds of formula (II-B) wherein G¹ is oxygen, R¹ is hydrogen, R² is G.075, X¹, X², X³ and X4 have the values listed in the table Q.

### Table 192 Q:

Table 192 Q provides 54 compounds of formula (II-B) wherein G¹ is oxygen, R¹ is hydrogen, R² is G.076, X¹, X², X³ and X4 have the values listed in the table Q.

### Table 193 Q:

Table 193 Q provides 54 compounds of formula (II-B) wherein G¹ is oxygen, R¹ is hydrogen, R² is G.077, X¹, X², X³ and X4 have the values listed in the table Q.

### Table 194 Q:

Table 194 Q provides 54 compounds of formula (II-B) wherein G¹ is oxygen, R¹ is hydrogen, R² is G.078, X¹, X², X³ and X4 have the values listed in the table Q.

### Table 195 Q:

Table 195 Q provides 54 compounds of formula (II-B) wherein G¹ is oxygen, R¹ is hydrogen, R² is G.079, X¹, X², X³ and X4 have the values listed in the table Q.

### Table 196 Q:

Table 196 Q provides 54 compounds of formula (II-B) wherein G¹ is oxygen, R¹ is hydrogen, R² is G.080, X¹, X², X³ and X4 have the values listed in the table Q.

### Table 197 Q:

Table 197 Q provides 54 compounds of formula (II-B) wherein G¹ is oxygen, R¹ is hydrogen, R² is G.081, X¹, X², X³ and X4 have the values listed in the table Q.

### Table 198 Q:

Table 198 Q provides 54 compounds of formula (II-B) wherein G¹ is oxygen, R¹ is hydrogen, R² is G.082, X¹, X², X³ and X4 have the values listed in the table Q.

### Table 199 Q:

Table 199 Q provides 54 compounds of formula (II-B) wherein G¹ is oxygen, R¹ is hydrogen, R² is G.083, X¹, X², X³ and X4 have the values listed in the table Q.

### Table 200 Q:

Table 200 Q provides 54 compounds of formula (II-B) wherein G¹ is oxygen, R¹ is hydrogen, R² is G.084, X¹, X², X³ and X4 have the values listed in the table Q.

### Table 201 Q:

Table 201 Q provides 54 compounds of formula (II-B) wherein G¹ is oxygen, R¹ is hydrogen, R² is G.085, X¹, X², X³ and X4 have the values listed in the table Q.

### Table 202 Q:

Table 202 Q provides 54 compounds of formula (II-B) wherein G¹ is oxygen, R¹ is hydrogen, R² is G.086, X¹, X², X³ and X4 have the values listed in the table Q.

### Table 203 Q:

Table 203 Q provides 54 compounds of formula (II-B) wherein G¹ is oxygen, R¹ is hydrogen R² is G.087, X¹, X², X³ and X4 have the values listed in the table Q.

### Table 204 Q:

Table 204 Q provides 54 compounds of formula (II-B) wherein G¹ is oxygen, R¹ is hydrogen, R² is G.088, X¹, X², X³ and X4 have the values listed in the table Q.

### Table 205 Q:

Table 205 Q provides 54 compounds of formula (II-B) wherein G¹ is oxygen, R¹ is hydrogen, R² is G.089, X¹, X², X³ and X4 have the values listed in the table Q.

### Table 206 Q:

Table 206 Q provides 54 compounds of formula (II-B) wherein G¹ is oxygen, R¹ is hydrogen R² is G.090, X¹, X², X³ and X4 have the values listed in the table Q.

### Table 207 Q:

Table 207 Q provides 54 compounds of formula (II-B) wherein G¹ is oxygen, R¹ is hydrogen, R² is G.091, X¹, X², X³ and X4 have the values listed in the table Q.

### Table 208 Q:

Table 208 Q provides 54 compounds of formula (II-B) wherein G¹ is oxygen, R¹ is hydrogen, R² is G.092, X¹, X², X³ and X4 have the values listed in the table Q.

### Table 209 Q:

Table 209 Q provides 54 compounds of formula (II-B) wherein G¹ is oxygen, R¹ is hydrogen, R² is G.093, X¹, X², X³ and X4 have the values listed in the table Q.

### Table 210 Q:

Table 210 Q provides 54 compounds of formula (II-B) wherein G¹ is oxygen, R¹ is hydrogen, R² is G.094, X¹, X², X³ and X4 have the values listed in the table Q.

### Table 211 Q:

Table 211 Q provides 54 compounds of formula (II-B) wherein G¹ is oxygen, R¹ is hydrogen, R² is G.095, X¹, X², X³ and X4 have the values listed in the table Q.

### Table 212 Q:

Table 212 Q provides 54 compounds of formula (II-B) wherein G¹ is oxygen, R¹ is hydrogen, R² is G.096, X¹, X², X³ and X4 have the values listed in the table Q.

### Table 213 Q:

Table 213 Q provides 54 compounds of formula (II-B) wherein G¹ is oxygen, R¹ is hydrogen, R² is G.097, X¹, X², X³ and X4 have the values listed in the table Q.

### Table 214 Q:

Table 214 Q provides 54 compounds of formula (II-B) wherein G¹ is oxygen, R¹ is hydrogen, R² is G.098, X¹, X², X³ and X4 have the values listed in the table Q.

### Table 215 Q:

Table 215 Q provides 54 compounds of formula (II-B) wherein G¹ is oxygen, R¹ is hydrogen, R² is G.099, X¹, X², X³ and X4 have the values listed in the table Q.

### Table 216 Q:

Table 216 Q provides 54 compounds of formula (II-B) wherein G¹ is oxygen, R¹ is hydrogen, R² is G.100, X¹, X², X³ and X4 have the values listed in the table Q.

### Table 217 Q:

Table 217 Q provides 54 compounds of formula (II-B) wherein G¹ is oxygen, R¹ is hydrogen, R² is G.101, X¹, X², X³ and X4 have the values listed in the table Q.

### Table 218 Q:

Table 218 Q provides 54 compounds of formula (II-B) wherein G¹ is oxygen, R¹ is hydrogen, R² is G.102, X¹, X², X³ and X4 have the values listed in the table Q.

### Table 219 Q:

Table 219 Q provides 54 compounds of formula (II-B) wherein G¹ is oxygen, R¹ is hydrogen, R² is G.103, X¹, X², X³ and X4 have the values listed in the table Q.

### Table 220 Q:

Table 220 Q provides 54 compounds of formula (II-B) wherein G¹ is oxygen, R¹ is hydrogen, R² is G.104, X¹, X², X³ and X4 have the values listed in the table Q.

### Table 221 Q:

Table 221 Q provides 54 compounds of formula (II-B) wherein G¹ is oxygen, R¹ is hydrogen, R² is G.105, X¹, X², X³ and X4 have the values listed in the table Q.

### Table 222 Q:

Table 222 Q provides 54 compounds of formula (II-B) wherein G¹ is oxygen, R¹ is hydrogen, R² is G.106, X¹, X², X³ and X4 have the values listed in the table Q.

### Table 223 Q:

Table 223 Q provides 54 compounds of formula (II-B) wherein G¹ is oxygen, R¹ is hydrogen, R² is G.107, X¹, X², X³ and X4 have the values listed in the table Q.

### Table 224 Q:

Table 224 Q provides 54 compounds of formula (II-B) wherein G¹ is oxygen, R¹ is hydrogen, R² is G.108, X¹, X², X³ and X4 have the values listed in the table Q.

### Table 225 Q:

Table 225 Q provides 54 compounds of formula (II-B) wherein G¹ is oxygen, R¹ is hydrogen, R² is G.109, X¹, X², X³ and X4 have the values listed in the table Q.

### Table 226 Q:

Table 226 Q provides 54 compounds of formula (II-B) wherein G¹ is oxygen, R¹ is hydrogen, R² is G.110, X¹, X², X³ and X4 have the values listed in the table Q.

### Table 227 Q:

Table 227 Q provides 54 compounds of formula (II-B) wherein G¹ is oxygen, R¹ is hydrogen, R² is G.111, X¹, X², X³ and X4 have the values listed in the table Q.

### Table 228 Q:

Table 228 Q provides 54 compounds of formula (II-B) wherein G¹ is oxygen, R¹ is hydrogen, R² is G.112, X¹, X², X³ and X4 have the values listed in the table Q.

### Table 229 Q:

Table 229 Q provides 54 compounds of formula (II-B) wherein G¹ is oxygen, R¹ is hydrogen, R² is G.113, X¹, X², X³ and X4 have the values listed in the table Q.

### Table 230 Q:

Table 230 Q provides 54 compounds of formula (II-B) wherein G¹ is oxygen, R¹ is hydrogen, R² is G.114, X¹, X², X³ and X4 have the values listed in the table Q.

### Table 231 Q:

Table 231 Q provides 54 compounds of formula (II-B) wherein G¹ is oxygen, R¹ is hydrogen, R² is G.115, X¹, X², X³ and X4 have the values listed in the table Q.

### Table 232 Q:

Table 232 Q provides 54 compounds of formula (II-B) wherein G¹ is oxygen, R¹ is hydrogen, R² is G.116, X¹, X², X³ and X4 have the values listed in the table Q.

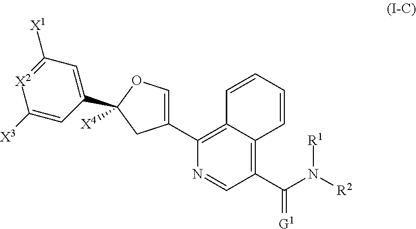

### Table 233Q:

Table 233 provides 54 compounds of formula (I-C) wherein G¹ is oxygen, R¹ is hydrogen, R² is G.001, X¹, X², X³ and X4 have the values listed in the table Q.

### Table 234 Q:

Table 234Q provides 54 compounds of formula (I-C) wherein G¹ is oxygen, R¹ is hydrogen, R² is G.002, X¹, X², X³ and X4 have the values listed in the table Q.

### Table 235 Q:

Table 235Q provides 54 compounds of formula (I-C) wherein G¹ is oxygen, R¹ is hydrogen, R² is G.003, X¹, X², X³ and X4 have the values listed in the table Q.

### Table 236 Q:

Table 236Q provides 54 compounds of formula (I-C) wherein G¹ is oxygen, R¹ is hydrogen, R² is G.004, X¹, X², X³ and X4 have the values listed in the table Q.

### Table 237 Q:

Table 237Q provides 54 compounds of formula (I-C) wherein G¹ is oxygen, R¹ is hydrogen, R² is G.005, X¹, X², X³ and X4 have the values listed in the table Q.

### Table 238 Q:

Table 238Q provides 54 compounds of formula (I-C) wherein G¹ is oxygen, R¹ is hydrogen, R² is G.006, X¹, X², X³ and X4 have the values listed in the table Q.

### Table 239 Q:

Table 239Q provides 54 compounds of formula (I-C) wherein G¹ is oxygen, R¹ is hydrogen, R² is G.007, X¹, X², X³ and X4 have the values listed in the table Q.

### Table 240 Q:

Table 240Q provides 54 compounds of formula (I-C) wherein G¹ is oxygen, R¹ is hydrogen, R² is G.008, X¹, X², X³ and X4 have the values listed in the table Q.

### Table 241 Q:

Table 241Q provides 54 compounds of formula (I-C) wherein G¹ is oxygen, R¹ is hydrogen, R² is G.009, X¹, X², X³ and X4 have the values listed in the table Q.

### Table 242 Q:

Table 242Q provides 54 compounds of formula (I-C) wherein G¹ is oxygen, R¹ is hydrogen, R² is G.010, X¹, X², X³ and X4 have the values listed in the table Q.

### Table 243 Q:

Table 243Q provides 54 compounds of formula (I-C) wherein G¹ is oxygen, R¹ is hydrogen, R² is G.011, X¹, X², X³ and X4 have the values listed in the table Q.

### Table 244 Q:

Table 244Q provides 54 compounds of formula (I-C) wherein G¹ is oxygen, R¹ is hydrogen, R² is G.012, X¹, X², X³ and X4 have the values listed in the table Q.

### Table 245 Q:

Table 245Q provides 54 compounds of formula (I-C) wherein G¹ is oxygen, R¹ is hydrogen, R² is G.013, X¹, X², X³ and X4 have the values listed in the table Q.

### Table 246 Q:

Table 246Q provides 54 compounds of formula (I-C) wherein G¹ is oxygen, R¹ is hydrogen, R² is G.014, X¹, X², X³ and X4 have the values listed in the table Q.

### Table 247 Q:

Table 247Q provides 54 compounds of formula (I-C) wherein G¹ is oxygen, R¹ is hydrogen, R² is G.015, X¹, X², X³ and X4 have the values listed in the table Q.

### Table 248 Q:

Table 248Q provides 54 compounds of formula (I-C) wherein G¹ is oxygen, R¹ is hydrogen, R² is G.016, X¹, X², X³ and X4 have the values listed in the table Q.

### Table 249 Q:

Table 249Q provides 54 compounds of formula (I-C) wherein G¹ is oxygen, R¹ is hydrogen, R² is G.017, X¹, X², X³ and X4 have the values listed in the table Q.

### Table 250 Q:

Table 250Q provides 54 compounds of formula (I-C) wherein G¹ is oxygen, R¹ is hydrogen, R² is G.018, X¹, X², X³ and X4 have the values listed in the table Q.

### Table 251 Q:

Table 251Q provides 54 compounds of formula (I-C) wherein G¹ is oxygen, R¹ is hydrogen, R² is G.019, X¹, X², X³ and X4 have the values listed in the table Q.

### Table 252 Q:

Table 252Q provides 54 compounds of formula (I-C) wherein G¹ is oxygen, R¹ is hydrogen, R² is G.020, X¹, X², X³ and X4 have the values listed in the table Q.

### Table 253 Q:

Table 253Q provides 54 compounds of formula (I-C) wherein G¹ is oxygen, R¹ is hydrogen, R² is G.021, X¹, X², X³ and X4 have the values listed in the table Q.

### Table 254Q:

Table 254Q provides 54 compounds of formula (I-C) wherein G¹ is oxygen, R¹ is hydrogen, R² is G.022, X¹, X², X³ and X4 have the values listed in the table Q.

### Table 255 Q:

Table 255Q provides 54 compounds of formula (I-C) wherein G¹ is oxygen, R¹ is hydrogen, R² is G.023, X¹, X², X³ and X4 have the values listed in the table Q.

### Table 256 Q:

Table 256Q provides 54 compounds of formula (I-C) wherein G¹ is oxygen, R¹ is hydrogen, R² is G.024, X¹, X², X³ and X4 have the values listed in the table Q.

### Table 257 Q:

Table 257Q provides 54 compounds of formula (I-C) wherein G¹ is oxygen, R¹ is hydrogen, R² is G.025, X¹, X², X³ and X4 have the values listed in the table Q.

### Table 258 Q:

Table 258Q provides 54 compounds of formula (I-C) wherein G¹ is oxygen, R¹ is hydrogen, R² is G.026, X¹, X², X³ and X4 have the values listed in the table Q.

### Table 259 Q:

Table 259Q provides 54 compounds of formula (I-C) wherein G¹ is oxygen, R¹ is hydrogen, R² is G.027, X¹, X², X³ and X4 have the values listed in the table Q.

### Table 260 Q:

Table 260Q provides 54 compounds of formula (I-C) wherein G¹ is oxygen, R¹ is hydrogen, R² is G.028, X¹, X², X³ and X4 have the values listed in the table Q.

### Table 261 Q:

Table 261Q provides 54 compounds of formula (I-C) wherein G¹ is oxygen, R¹ is hydrogen, R² is G.029, X¹, X², X³ and X4 have the values listed in the table Q.

### Table 262 Q:

Table 262Q provides 54 compounds of formula (I-C) wherein G¹ is oxygen, R¹ is hydrogen, R² is G.030, X¹, X², X³ and X4 have the values listed in the table Q.

### Table 263 Q:

Table 263Q provides 54 compounds of formula (I-C) wherein G¹ is oxygen, R¹ is hydrogen, R² is G.031, X¹, X², X³ and X4 have the values listed in the table Q.

### Table 264 Q:

Table 264Q provides 54 compounds of formula (I-C) wherein G¹ is oxygen, R¹ is hydrogen, R² is G.032, X¹, X², X³ and X4 have the values listed in the table Q.

### Table 265 Q:

Table 265Q provides 54 compounds of formula (I-C) wherein G¹ is oxygen, R¹ is hydrogen, R² is G.033, X¹, X², X³ and X4 have the values listed in the table Q.

### Table 266 Q:

Table 266Q provides 54 compounds of formula (I-C) wherein G¹ is oxygen, R¹ is hydrogen, R² is G.034, X¹, X², X³ and X4 have the values listed in the table Q.

### Table 267 Q:

Table 267Q provides 54 compounds of formula (I-C) wherein G¹ is oxygen, R¹ is hydrogen, R² is G.035, X¹, X², X³ and X4 have the values listed in the table Q.

### Table 268 Q:

Table 268Q provides 54 compounds of formula (I-C) wherein G¹ is oxygen, R¹ is hydrogen, R² is G.036, X¹, X², X³ and X4 have the values listed in the table Q.

### Table 269 Q:

Table 269Q provides 54 compounds of formula (I-C) wherein G¹ is oxygen, R¹ is hydrogen, R² is G.037, X¹, X², X³ and X4 have the values listed in the table Q.

### Table 270 Q:

Table 270Q provides 54 compounds of formula (I-C) wherein G¹ is oxygen, R¹ is hydrogen, R² is G.038, X¹, X², X³ and X4 have the values listed in the table Q.

### Table 271 Q:

Table 271Q provides 54 compounds of formula (I-C) wherein G¹ is oxygen, R¹ is hydrogen, R² is G.039, X¹, X², X³ and X4 have the values listed in the table Q.

### Table 272 Q:

Table 272Q provides 54 compounds of formula (I-C) wherein G¹ is oxygen, R¹ is hydrogen, R² is G.040, X¹, X², X³ and X4 have the values listed in the table Q.

### Table 273 Q:

Table 273Q provides 54 compounds of formula (I-C) wherein G¹ is oxygen, R¹ is hydrogen, R² is G.041, X¹, X², X³ and X4 have the values listed in the table Q.

### Table 274 Q:

Table 274Q provides 54 compounds of formula (I-C) wherein G¹ is oxygen, R¹ is hydrogen, R² is G.042, X¹, X², X³ and X4 have the values listed in the table Q.

### Table 275 Q:

Table 275Q provides 54 compounds of formula (I-C) wherein G¹ is oxygen, R¹ is hydrogen, R² is G.043, X¹, X², X³ and X4 have the values listed in the table Q.

### Table 276 Q:

Table 276Q provides 54 compounds of formula (I-C) wherein G¹ is oxygen, R¹ is hydrogen, R² is G.044, X¹, X², X³ and X4 have the values listed in the table Q.

### Table 277 Q:

Table 277Q provides 54 compounds of formula (I-C) wherein G¹ is oxygen, R¹ is hydrogen, R² is G.045, X¹, X², X³ and X4 have the values listed in the table Q.

### Table 278 Q:

Table 278Q provides 54 compounds of formula (I-C) wherein G¹ is oxygen, R¹ is hydrogen, R² is G.046, X¹, X², X³ and X4 have the values listed in the table Q.

### Table 279 Q:

Table 279Q provides 54 compounds of formula (I-C) wherein G¹ is oxygen, R¹ is hydrogen, R² is G.047, X¹, X², X³ and X4 have the values listed in the table Q.

### Table 280 Q:

Table 280Q provides 54 compounds of formula (I-C) wherein G¹ is oxygen, R¹ is hydrogen, R² is G.048, X¹, X², X³ and X4 have the values listed in the table Q.

### Table 281 Q:

Table 281Q provides 54 compounds of formula (I-C) wherein G¹ is oxygen, R¹ is hydrogen, R² is G.049, X¹, X², X³ and X4 have the values listed in the table Q.

### Table 282 Q:

Table 282Q provides 54 compounds of formula (I-C) wherein G¹ is oxygen, R¹ is hydrogen, R² is G.050, X¹, X², X³ and X4 have the values listed in the table Q.

### Table 283 Q:

Table 283Q provides 54 compounds of formula (I-C) wherein G¹ is oxygen, R¹ is hydrogen, R² is G.051, X¹, X², X³ and X4 have the values listed in the table Q.

### Table 284 Q:

Table 284Q provides 54 compounds of formula (I-C) wherein G¹ is oxygen, R¹ is hydrogen, R² is G.052, X¹, X², X³ and X4 have the values listed in the table Q.

### Table 285 Q:

Table 285Q provides 54 compounds of formula (I-C) wherein G¹ is oxygen, R¹ is hydrogen, R² is G.053, X¹, X², X³ and X4 have the values listed in the table Q.

### Table 286 Q:

Table 286Q provides 54 compounds of formula (I-C) wherein G¹ is oxygen, R¹ is hydrogen, R² is G.054, X¹, X², X³ and X4 have the values listed in the table Q.

### Table 287 Q:

Table 287Q provides 54 compounds of formula (I-C) wherein G¹ is oxygen, R¹ is hydrogen, R² is G.055, X¹, X², X³ and X4 have the values listed in the table Q.

### Table 288 Q:

Table 288Q provides 54 compounds of formula (I-C) wherein G¹ is oxygen, R¹ is hydrogen, R² is G.056, X¹, X², X³ and X4 have the values listed in the table Q.

### Table 289 Q:

Table 289Q provides 54 compounds of formula (I-C) wherein G¹ is oxygen, R¹ is hydrogen, R² is G.057, X¹, X², X³ and X4 have the values listed in the table Q.

### Table 290 Q:

Table 290Q provides 54 compounds of formula (I-C) wherein G¹ is oxygen, R¹ is hydrogen, R² is G.058, X¹, X², X³ and X4 have the values listed in the table Q.

### Table 291 Q:

Table 291Q provides 54 compounds of formula (I-C) wherein G¹ is oxygen, R¹ is hydrogen, R² is G.059, X¹, X², X³ and X4 have the values listed in the table Q.

### Table 292 Q:

Table 292Q provides 54 compounds of formula (I-C) wherein G¹ is oxygen, R¹ is hydrogen, R² is G.060, X¹, X², X³ and X4 have the values listed in the table Q.

### Table 293 Q:

Table 293Q provides 54 compounds of formula (I-C) wherein G¹ is oxygen, R¹ is hydrogen, R² is G.061, X¹, X², X³ and X4 have the values listed in the table Q.

### Table 294 Q:

Table 294Q provides 54 compounds of formula (I-C) wherein G¹ is oxygen, R¹ is hydrogen, R² is G.062, X¹, X², X³ and X4 have the values listed in the table Q.

### Table 295 Q:

Table 295Q provides 54 compounds of formula (I-C) wherein G¹ is oxygen, R¹ is hydrogen, R² is G.063, X¹, X², X³ and X4 have the values listed in the table Q.

### Table 296 Q:

Table 296Q provides 54 compounds of formula (I-C) wherein G¹ is oxygen, R¹ is hydrogen, R² is G.064, X¹, X², X³ and X4 have the values listed in the table Q.

### Table 297 Q:

Table 297Q provides 54 compounds of formula (I-C) wherein G¹ is oxygen, R¹ is hydrogen, R² is G.065, X¹, X², X³ and X4 have the values listed in the table Q.

### Table 298 Q:

Table 298Q provides 54 compounds of formula (I-C) wherein G¹ is oxygen, R¹ is hydrogen, R² is G.066, X¹, X², X³ and X4 have the values listed in the table Q.

### Table 299 Q:

Table 299Q provides 54 compounds of formula (I-C) wherein G¹ is oxygen, R¹ is hydrogen, R² is G.067, X¹, X², X³ and X4 have the values listed in the table Q.

### Table 300 Q:

Table 300Q provides 54 compounds of formula (I-C) wherein G¹ is oxygen, R¹ is hydrogen, R² is G.068, X¹, X², X³ and X4 have the values listed in the table Q.
Table 301Q provides 54 compounds of formula (I-C) wherein G¹ is oxygen, R¹ is hydrogen, R² is G.069, X¹, X², X³ and X4 have the values listed in the table Q.

### Table 302 Q:

Table 302Q provides 54 compounds of formula (I-C) wherein G¹ is oxygen, R¹ is hydrogen, R² is G.070, X¹, X², X³ and X4 have the values listed in the table Q.

### Table 303 Q:

Table 303Q provides 54 compounds of formula (I-C) wherein G¹ is oxygen, R¹ is hydrogen, R² is G.071, X¹, X², X³ and X4 have the values listed in the table Q.

### Table 304 Q:

Table 304Q provides 54 compounds of formula (I-C) wherein G¹ is oxygen, R¹ is hydrogen, R² is G.072, X¹, X², X³ and X4 have the values listed in the table Q.

### Table 305 Q:

Table 305Q provides 54 compounds of formula (I-C) wherein G¹ is oxygen, R¹ is hydrogen, R² is G.073, X¹, X², X³ and X4 have the values listed in the table Q.

### Table 306 Q:

Table 306Q provides 54 compounds of formula (I-C) wherein G¹ is oxygen, R¹ is hydrogen, R² is G.074, X¹, X², X³ and X4 have the values listed in the table Q.

### Table 307 Q:

Table 307Q provides 54 compounds of formula (I-C) wherein G¹ is oxygen, R¹ is hydrogen, R² is G.075, X¹, X², X³ and X4 have the values listed in the table Q.

### Table 308 Q:

Table 308Q provides 54 compounds of formula (I-C) wherein G¹ is oxygen, R¹ is hydrogen, R² is G.076, X¹, X², X³ and X4 have the values listed in the table Q.

### Table 309 Q:

Table 309Q provides 54 compounds of formula (I-C) wherein G¹ is oxygen, R¹ is hydrogen, R² is G.077, X¹, X², X³ and X4 have the values listed in the table Q.

### Table 310 Q:

Table 310Q provides 54 compounds of formula (I-C) wherein G¹ is oxygen, R¹ is hydrogen, R² is G.078, X¹, X², X³ and X4 have the values listed in the table Q.

### Table 311 Q:

Table 311Q provides 54 compounds of formula (I-C) wherein G¹ is oxygen, R¹ is hydrogen, R² is G.079, X¹, X², X³ and X4 have the values listed in the table Q.

### Table 312 Q:

Table 312Q provides 54 compounds of formula (I-C) wherein G¹ is oxygen, R¹ is hydrogen, R² is G.080, X¹, X², X³ and X4 have the values listed in the table Q.

### Table 313 Q:

Table 313Q provides 54 compounds of formula (I-C) wherein G¹ is oxygen, R¹ is hydrogen, R² is G.081, X¹, X², X³ and X4 have the values listed in the table Q.

### Table 314 Q:

Table 314Q provides 54 compounds of formula (I-C) wherein G¹ is oxygen, R¹ is hydrogen, R² is G.082, X¹, X², X³ and X4 have the values listed in the table Q.

### Table 315 Q:

Table 315Q provides 54 compounds of formula (I-C) wherein G¹ is oxygen, R¹ is hydrogen, R² is G.083, X¹, X², X³ and X4 have the values listed in the table Q.

### Table 316 Q:

Table 316Q provides 54 compounds of formula (I-C) wherein G¹ is oxygen, R¹ is hydrogen, R² is G.084, X¹, X², X³ and X4 have the values listed in the table Q.

### Table 317 Q:

Table 317Q provides 54 compounds of formula (I-C) wherein G¹ is oxygen, R¹ is hydrogen, R² is G.085, X¹, X², X³ and X4 have the values listed in the table Q.

### Table 318 Q:

Table 318Q provides 54 compounds of formula (I-C) wherein G¹ is oxygen, R¹ is hydrogen, R² is G.086, X¹, X², X³ and X4 have the values listed in the table Q.

### Table 319 Q:

Table 319Q provides 54 compounds of formula (I-C) wherein G¹ is oxygen, R¹ is hydrogen, R² is G.087, X¹, X², X³ and X4 have the values listed in the table Q.

### Table 320 Q:

Table 320Q provides 54 compounds of formula (I-C) wherein G¹ is oxygen, R¹ is hydrogen, R² is G.088, X¹, X², X³ and X4 have the values listed in the table Q.

### Table 321 Q:

Table 312Q provides 54 compounds of formula (I-C) wherein G¹ is oxygen, R¹ is hydrogen, R² is G.089, X¹, X², X³ and X4 have the values listed in the table Q.

### Table 322 Q:

Table 322Q provides 54 compounds of formula (I-C) wherein G¹ is oxygen, R¹ is hydrogen, R² is G.090, X¹, X², X³ and X4 have the values listed in the table Q.

### Table 323 Q:

Table 323Q provides 54 compounds of formula (I-C) wherein G¹ is oxygen, R¹ is hydrogen, R² is G.091, X¹, X², X³ and X4 have the values listed in the table Q.

### Table 324 Q:

Table 324Q provides 54 compounds of formula (I-C) wherein G¹ is oxygen, R¹ is hydrogen, R² is G.092, X¹, X², X³ and X4 have the values listed in the table Q.

### Table 325 Q:

Table 325Q provides 54 compounds of formula (I-C) wherein G¹ is oxygen, R¹ is hydrogen, R² is G.093, X¹, X², X³ and X4 have the values listed in the table Q.

### Table 326 Q:

Table 326Q provides 54 compounds of formula (I-C) wherein G¹ is oxygen, R¹ is hydrogen, R² is G.094, X¹, X², X³ and X4 have the values listed in the table Q.

### Table 327 Q:

Table 327Q provides 54 compounds of formula (I-C) wherein G¹ is oxygen, R¹ is hydrogen, R² is G.095, X¹, X², X³ and X4 have the values listed in the table Q.

### Table 328 Q:

Table 328Q provides 54 compounds of formula (I-C) wherein G¹ is oxygen, R¹ is hydrogen, R² is G.096, X¹, X², X³ and X4 have the values listed in the table Q.

### Table 329 Q:

Table 329Q provides 54 compounds of formula (I-C) wherein G¹ is oxygen, R¹ is hydrogen, R² is G.097, X¹, X², X³ and X4 have the values listed in the table Q.

### Table 330 Q:

Table 330Q provides 54 compounds of formula (I-C) wherein G¹ is oxygen, R¹ is hydrogen, R² is G.098, X¹, X², X³ and X4 have the values listed in the table Q.

### Table 331 Q:

Table 331Q provides 54 compounds of formula (I-C) wherein G¹ is oxygen, R¹ is hydrogen, R² is G.099, X¹, X², X³ and X4 have the values listed in the table Q.

### Table 332 Q:

Table 332Q provides 54 compounds of formula (I-C) wherein G¹ is oxygen, R¹ is hydrogen, R² is G.100, X¹, X², X³ and X4 have the values listed in the table Q.

### Table 333 Q:

Table 333Q provides 54 compounds of formula (I-C) wherein G¹ is oxygen, R¹ is hydrogen, R² is G.101, X¹, X², X³ and X4 have the values listed in the table Q.

### Table 334 Q:

Table 334Q provides 54 compounds of formula (I-C) wherein G¹ is oxygen, R¹ is hydrogen, R² is G.102, X¹, X², X³ and X4 have the values listed in the table Q.

### Table 335 Q:

Table 335Q provides 54 compounds of formula (I-C) wherein G¹ is oxygen, R¹ is hydrogen, R² is G.103, X¹, X², X³ and X4 have the values listed in the table Q.

### Table 336 Q:

Table 336Q provides 54 compounds of formula (I-C) wherein G¹ is oxygen, R¹ is hydrogen, R² is G.104, X¹, X², X³ and X4 have the values listed in the table Q.

### Table 337 Q:

Table 337Q provides 54 compounds of formula (I-C) wherein G¹ is oxygen, R¹ is hydrogen, R² is G.105, X¹, X², X³ and X4 have the values listed in the table Q.

### Table 338 Q:

Table 338Q provides 54 compounds of formula (I-C) wherein G¹ is oxygen, R¹ is hydrogen, R² is G.106, X¹, X², X³ and X4 have the values listed in the table Q.

### Table 339 Q:

Table 339Q provides 54 compounds of formula (I-C) wherein G¹ is oxygen, R¹ is hydrogen, R² is G.107, X¹, X², X³ and X4 have the values listed in the table Q.

### Table 340 Q:

Table 340Q provides 54 compounds of formula (I-C) wherein G¹ is oxygen, R¹ is hydrogen, R² is G.108, X¹, X², X³ and X4 have the values listed in the table Q.

### Table 341 Q:

Table 341Q provides 54 compounds of formula (I-C) wherein G¹ is oxygen, R¹ is hydrogen, R² is G.109, X¹, X², X³ and X4 have the values listed in the table Q.

### Table 342 Q:

Table 342Q provides 54 compounds of formula (I-C) wherein G¹ is oxygen, R¹ is hydrogen, R² is G.110, X¹, X², X³ and X4 have the values listed in the table Q.

### Table 343 Q:

Table 343Q provides 54 compounds of formula (I-C) wherein G¹ is oxygen, R¹ is hydrogen, R² is G.111, X¹, X², X³ and X4 have the values listed in the table Q.

### Table 344 Q:

Table 344Q provides 54 compounds of formula (I-C) wherein G¹ is oxygen, R¹ is hydrogen, R² is G.112, X¹, X², X³ and X4 have the values listed in the table Q.

### Table 345 Q:

Table 345Q provides 54 compounds of formula (I-C) wherein G¹ is oxygen, R¹ is hydrogen, R² is G.113, X¹, X², X³ and X4 have the values listed in the table Q.

### Table 346 Q:

Table 346Q provides 54 compounds of formula (I-C) wherein G¹ is oxygen, R¹ is hydrogen, R² is G.114, X¹, X², X³ and X4 have the values listed in the table Q.

### Table 347 Q:

Table 347Q provides 54 compounds of formula (I-C) wherein G¹ is oxygen, R¹ is hydrogen, R² is G.115, X¹, X², X³ and X4 have the values listed in the table Q.

### Table 348 Q:

Table 348Q provides 54 compounds of formula (I-C) wherein G¹ is oxygen, R¹ is hydrogen, R² is G.116, X¹, X², X³ and X4 have the values listed in the table Q.

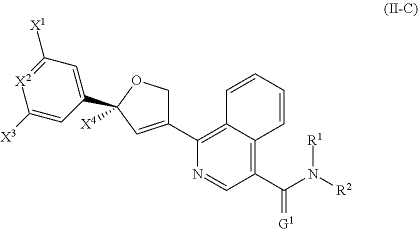

### Table 349 Q:

Table 349Q provides 54 compounds of formula (II-C) wherein G¹ is oxygen, R¹ is hydrogen, R² is G.001, X¹, X², X³ and X4 have the values listed in the table Q.

### Table 350 Q:

Table 350Q provides 54 compounds of formula (II-C) wherein G¹ is oxygen, R¹ is hydrogen, R² is G.002, X¹, X², X³ and X4 have the values listed in the table Q.

### Table 351 Q:

Table 351Q provides 54 compounds of formula (II-C) wherein G¹ is oxygen, R¹ is hydrogen, R² is G.003, X¹, X², X³ and X4 have the values listed in the table Q.

### Table 352 Q:

Table 352Q provides 54 compounds of formula (II-C) wherein G¹ is oxygen, R¹ is hydrogen, R² is G.004, X¹, X², X³ and X4 have the values listed in the table Q.

### Table 353 Q:

Table 353Q provides 54 compounds of formula (II-C) wherein G¹ is oxygen, R¹ is hydrogen, R² is G.005, X¹, X², X³ and X4 have the values listed in the table Q.

### Table 354 Q:

Table 354Q provides 54 compounds of formula (II-C) wherein G¹ is oxygen, R¹ is hydrogen, R² is G.006, X¹, X², X³ and X4 have the values listed in the table Q.

### Table 355 Q:

Table 355Q provides 54 compounds of formula (II-C) wherein G¹ is oxygen, R¹ is hydrogen, R² is G.007, X¹, X², X³ and X4 have the values listed in the table Q.

### Table 356 Q:

Table 356Q provides 54 compounds of formula (II-C) wherein G¹ is oxygen, R¹ is hydrogen, R² is G.008, X¹, X², X³ and X4 have the values listed in the table Q.

### Table 357 Q:

Table 357Q provides 54 compounds of formula (II-C) wherein G¹ is oxygen, R¹ is hydrogen, R² is G.009, X¹, X², X³ and X4 have the values listed in the table Q.

### Table 358 Q:

Table 358Q provides 54 compounds of formula (II-C) wherein G¹ is oxygen, R¹ is hydrogen, R² is G.010, X¹, X², X³ and X4 have the values listed in the table Q.

### Table 359 Q:

Table 359Q provides 54 compounds of formula (II-C) wherein G¹ is oxygen, R¹ is hydrogen, R² is G.011, X¹, X², X³ and X4 have the values listed in the table Q.

### Table 360 Q:

Table 360Q provides 54 compounds of formula (II-C) wherein G¹ is oxygen, R¹ is hydrogen, R² is G.012, X¹, X², X³ and X4 have the values listed in the table Q.

### Table 361 Q:

Table 361Q provides 54 compounds of formula (II-C) wherein G¹ is oxygen, R¹ is hydrogen, R² is G.013, X¹, X², X³ and X4 have the values listed in the table Q.

### Table 362 Q:

Table 362Q provides 54 compounds of formula (II-C) wherein G¹ is oxygen, R¹ is hydrogen, R² is G.014, X¹, X², X³ and X4 have the values listed in the table Q.

### Table 363 Q:

Table 363Q provides 54 compounds of formula (II-C) wherein G¹ is oxygen, R¹ is hydrogen, R² is G.015, X¹, X², X³ and X4 have the values listed in the table Q.

### Table 364 Q:

Table 364Q provides 54 compounds of formula (II-C) wherein G¹ is oxygen, R¹ is hydrogen, R² is G.016, X¹, X², X³ and X4 have the values listed in the table Q.

### Table 365 Q:

Table 365Q provides 54 compounds of formula (II-C) wherein G¹ is oxygen, R¹ is hydrogen, R² is G.017, X¹, X², X³ and X4 have the values listed in the table Q.

### Table 366 Q:

Table 366Q provides 54 compounds of formula (II-C) wherein G¹ is oxygen, R¹ is hydrogen, R² is G.018, X¹, X², X³ and X4 have the values listed in the table Q.

### Table 367 Q:

Table 367Q provides 54 compounds of formula (II-C) wherein G¹ is oxygen, R¹ is hydrogen, R² is G.019, X¹, X², X³ and X4 have the values listed in the table Q.

### Table 368 Q:

Table 368Q provides 54 compounds of formula (II-C) wherein G¹ is oxygen, R¹ is hydrogen, R² is G.020, X¹, X², X³ and X4 have the values listed in the table Q.

### Table 369 Q:

Table 369Q provides 54 compounds of formula (II-C) wherein G¹ is oxygen, R¹ is hydrogen, R² is G.021, X¹, X², X³ and X4 have the values listed in the table Q.

### Table 370 Q:

Table 370Q provides 54 compounds of formula (II-C) wherein G¹ is oxygen, R¹ is hydrogen, R² is G.022, X¹, X², X³ and X4 have the values listed in the table Q.

### Table 371 Q:

Table 371Q provides 54 compounds of formula (II-C) wherein G¹ is oxygen, R¹ is hydrogen, R² is G.023, X¹, X², X³ and X4 have the values listed in the table Q.

### Table 372 Q:

Table 372Q provides 54 compounds of formula (II-C) wherein G¹ is oxygen, R¹ is hydrogen, R² is G.024, X¹, X², X³ and X4 have the values listed in the table Q.

### Table 373 Q:

Table 373Q provides 54 compounds of formula (II-C) wherein G¹ is oxygen, R¹ is hydrogen, R² is G.025, X¹, X², X³ and X4 have the values listed in the table Q.

### Table 374 Q:

Table 374Q provides 54 compounds of formula (II-C) wherein G¹ is oxygen, R¹ is hydrogen, R² is G.026, X¹, X², X³ and X4 have the values listed in the table Q.

### Table 375 Q:

Table 375Q provides 54 compounds of formula (II-C) wherein G¹ is oxygen, R¹ is hydrogen, R² is G.027, X¹, X², X³ and X4 have the values listed in the table Q.

### Table 376 Q:

Table 376Q provides 54 compounds of formula (II-C) wherein G¹ is oxygen, R¹ is hydrogen, R² is G.028, X¹, X², X³ and X4 have the values listed in the table Q.

### Table 377 Q:

Table 377Q provides 54 compounds of formula (II-C) wherein G¹ is oxygen, R¹ is hydrogen, R² is G.029, X¹, X², X³ and X4 have the values listed in the table Q.

### Table 378 Q:

Table 378Q provides 54 compounds of formula (II-C) wherein G¹ is oxygen, R¹ is hydrogen, R² is G.030, X¹, X², X³ and X4 have the values listed in the table Q.

### Table 379 Q:

Table 379Q provides 54 compounds of formula (II-C) wherein G¹ is oxygen, R¹ is hydrogen, R² is G.031, X¹, X², X³ and X4 have the values listed in the table Q.

### Table 380 Q:

Table 380Q provides 54 compounds of formula (II-C) wherein G¹ is oxygen, R¹ is hydrogen, R² is G.032, X¹, X², X³ and X4 have the values listed in the table Q.

### Table 381 Q:

Table 381Q provides 54 compounds of formula (II-C) wherein G¹ is oxygen, R¹ is hydrogen, R² is G.033, X¹, X², X³ and X4 have the values listed in the table Q.

### Table 382 Q:

Table 382Q provides 54 compounds of formula (II-C) wherein G¹ is oxygen, R¹ is hydrogen, R² is G.034, X¹, X², X³ and X4 have the values listed in the table Q.

### Table 383 Q:

Table 383Q provides 54 compounds of formula (II-C) wherein G¹ is oxygen, R¹ is hydrogen, R² is G.035, X¹, X², X³ and X4 have the values listed in the table Q.

### Table 384 Q:

Table 384Q provides 54 compounds of formula (II-C) wherein G¹ is oxygen, R¹ is hydrogen, R² is G.036, X¹, X², X³ and X4 have the values listed in the table Q.

### Table 385 Q:

Table 385Q provides 54 compounds of formula (II-C) wherein G¹ is oxygen, R¹ is hydrogen, R² is G.037, X¹, X², X³ and X4 have the values listed in the table Q.

### Table 386 Q:

Table 386Q provides 54 compounds of formula (II-C) wherein G¹ is oxygen, R¹ is hydrogen, R² is G.038, X¹, X², X³ and X4 have the values listed in the table Q.

### Table 387 Q:

Table 387Q provides 54 compounds of formula (II-C) wherein G¹ is oxygen, R¹ is hydrogen, R² is G.039, X¹, X², X³ and X4 have the values listed in the table Q.

### Table 388 Q:

Table 388Q provides 54 compounds of formula (II-C) wherein G¹ is oxygen, R¹ is hydrogen, R² is G.040, X¹, X², X³ and X4 have the values listed in the table Q.

### Table 389 Q:

Table 389Q provides 54 compounds of formula (II-C) wherein G¹ is oxygen, R¹ is hydrogen, R² is G.041, X¹, X², X³ and X4 have the values listed in the table Q.

### Table 390 Q:

Table 390Q provides 54 compounds of formula (II-C) wherein G¹ is oxygen, R¹ is hydrogen, R² is G.042, X¹, X², X³ and X4 have the values listed in the table Q.

### Table 391 Q:

Table 391Q provides 54 compounds of formula (II-C) wherein G¹ is oxygen, R¹ is hydrogen, R² is G.043, X¹, X², X³ and X4 have the values listed in the table Q.

### Table 392 Q:

Table 392Q provides 54 compounds of formula (II-C) wherein G¹ is oxygen, R¹ is hydrogen, R² is G.044, X¹, X², X³ and X4 have the values listed in the table Q.

### Table 393 Q:

Table 393Q provides 54 compounds of formula (II-C) wherein G¹ is oxygen, R¹ is hydrogen, R² is G.045, X¹, X², X³ and X4 have the values listed in the table Q.

### Table 394 Q:

Table 394Q provides 54 compounds of formula (II-C) wherein G¹ is oxygen, R¹ is hydrogen, R² is G.046, X¹, X², X³ and X4 have the values listed in the table Q.

### Table 395 Q:

Table 395Q provides 54 compounds of formula (II-C) wherein G¹ is oxygen, R¹ is hydrogen, R² is G.047, X¹, X², X³ and X4 have the values listed in the table Q.

### Table 396 Q:

Table 396Q provides 54 compounds of formula (II-C) wherein G¹ is oxygen, R¹ is hydrogen, R² is G.048, X¹, X², X³ and X4 have the values listed in the table Q.

### Table 397 Q:

Table 397Q provides 54 compounds of formula (II-C) wherein G¹ is oxygen, R¹ is hydrogen, R² is G.049, X¹, X², X³ and X4 have the values listed in the table Q.

### Table 398 Q:

Table 398Q provides 54 compounds of formula (II-C) wherein G¹ is oxygen, R¹ is hydrogen, R² is G.050, X¹, X², X³ and X4 have the values listed in the table Q.

### Table 399 Q:

Table 399Q provides 54 compounds of formula (II-C) wherein G¹ is oxygen, R¹ is hydrogen, R² is G.051, X¹, X², X³ and X4 have the values listed in the table Q.

### Table 400 Q:

Table 400Q provides 54 compounds of formula (II-C) wherein G¹ is oxygen, R¹ is hydrogen, R² is G.052, X¹, X², X³ and X4 have the values listed in the table Q.

### Table 401 Q:

Table 401Q provides 54 compounds of formula (II-C) wherein G¹ is oxygen, R¹ is hydrogen, R² is G.053, X¹, X², X³ and X4 have the values listed in the table Q.

### Table 402 Q:

Table 402Q provides 54 compounds of formula (II-C) wherein G¹ is oxygen, R¹ is hydrogen, R² is G.054, X¹, X², X³ and X4 have the values listed in the table Q.

### Table 403 Q:

Table 403Q provides 54 compounds of formula (II-C) wherein G¹ is oxygen, R¹ is hydrogen, R² is G.055, X¹, X², X³ and X4 have the values listed in the table Q.

### Table 404 Q:

Table 404Q provides 54 compounds of formula (II-C) wherein G¹ is oxygen, R¹ is hydrogen, R² is G.056, X¹, X², X³ and X4 have the values listed in the table Q.

### Table 405 Q:

Table 405Q provides 54 compounds of formula (II-C) wherein G¹ is oxygen, R¹ is hydrogen, R² is G.057, X¹, X², X³ and X4 have the values listed in the table Q.

### Table 406 Q:

Table 406Q provides 54 compounds of formula (II-C) wherein G¹ is oxygen, R¹ is hydrogen, R² is G.058, X¹, X², X³ and X4 have the values listed in the table Q.

### Table 407 Q:

Table 407Q provides 54 compounds of formula (II-C) wherein G¹ is oxygen, R¹ is hydrogen, R² is G.059, X¹, X², X³ and X4 have the values listed in the table Q.

### Table 408 Q:

Table 408Q provides 54 compounds of formula (II-C) wherein G¹ is oxygen, R¹ is hydrogen, R² is G.060, X¹, X², X³ and X4 have the values listed in the table Q.

### Table 409 Q:

Table 409Q provides 54 compounds of formula (II-C) wherein G¹ is oxygen, R¹ is hydrogen, R² is G.061, X¹, X², X³ and X4 have the values listed in the table Q.

### Table 410 Q:

Table 410Q provides 54 compounds of formula (II-C) wherein G¹ is oxygen, R¹ is hydrogen, R² is G.062, X¹, X², X³ and X4 have the values listed in the table Q.

### Table 411 Q:

Table 411Q provides 54 compounds of formula (II-C) wherein G¹ is oxygen, R¹ is hydrogen, R² is G.063, X¹, X², X³ and X4 have the values listed in the table Q.

### Table 412 Q:

Table 412Q provides 54 compounds of formula (II-C) wherein G¹ is oxygen, R¹ is hydrogen, R² is G.064, X¹, X², X³ and X4 have the values listed in the table Q.

### Table 413 Q:

Table 413Q provides 54 compounds of formula (II-C) wherein G¹ is oxygen, R¹ is hydrogen, R² is G.065, X¹, X², X³ and X4 have the values listed in the table Q.

### Table 414 Q:

Table 414Q provides 54 compounds of formula (II-C) wherein G¹ is oxygen, R¹ is hydrogen, R² is G.066, X¹, X², X³ and X4 have the values listed in the table Q.

### Table 415 Q:

Table 415Q provides 54 compounds of formula (II-C) wherein G¹ is oxygen, R¹ is hydrogen, R² is G.067, X¹, X², X³ and X4 have the values listed in the table Q.

### Table 416 Q:

Table 416Q provides 54 compounds of formula (II-C) wherein G¹ is oxygen, R¹ is hydrogen, R² is G.068, X¹, X², X³ and X4 have the values listed in the table Q.

### Table 417 Q:

Table 417Q provides 54 compounds of formula (II-C) wherein G¹ is oxygen, R¹ is hydrogen, R² is G.069, X¹, X², X³ and X4 have the values listed in the table Q.

### Table 418 Q:

Table 418Q provides 54 compounds of formula (II-C) wherein G¹ is oxygen, R¹ is hydrogen, R² is G.070, X¹, X², X³ and X4 have the values listed in the table Q.

### Table 419 Q:

Table 419Q provides 54 compounds of formula (II-C) wherein G¹ is oxygen, R¹ is hydrogen, R² is G.071, X¹, X², X³ and X4 have the values listed in the table Q.

### Table 420 Q:

Table 420Q provides 54 compounds of formula (II-C) wherein G¹ is oxygen, R¹ is hydrogen, R² is G.072, X¹, X², X³ and X4 have the values listed in the table Q.

### Table 421 Q:

Table 421Q provides 54 compounds of formula (II-C) wherein G¹ is oxygen, R¹ is hydrogen, R² is G.073, X¹, X², X³ and X4 have the values listed in the table Q.

### Table 422 Q:

Table 422Q provides 54 compounds of formula (II-C) wherein G¹ is oxygen, R¹ is hydrogen, R² is G.074, X¹, X², X³ and X4 have the values listed in the table Q.

### Table 423 Q:

Table 423Q provides 54 compounds of formula (II-C) wherein G¹ is oxygen, R¹ is hydrogen, R² is G.075, X¹, X², X³ and X4 have the values listed in the table Q.

### Table 424 Q:

Table 424Q provides 54 compounds of formula (II-C) wherein G¹ is oxygen, R¹ is hydrogen, R² is G.076, X¹, X², X³ and X4 have the values listed in the table Q.

### Table 425 Q:

Table 425Q provides 54 compounds of formula (II-C) wherein G¹ is oxygen, R¹ is hydrogen, R² is G.077, X¹, X², X³ and X4 have the values listed in the table Q.

### Table 426 Q:

Table 426Q provides 54 compounds of formula (II-C) wherein G¹ is oxygen, R¹ is hydrogen, R² is G.078, X¹, X², X³ and X4 have the values listed in the table Q.

### Table 427 Q:

Table 427Q provides 54 compounds of formula (II-C) wherein G¹ is oxygen, R¹ is hydrogen, R² is G.079, X¹, X², X³ and X4 have the values listed in the table Q.

### Table 428 Q:

Table 428Q provides 54 compounds of formula (II-C) wherein G¹ is oxygen, R¹ is hydrogen, R² is G.080, X¹, X², X³ and X4 have the values listed in the table Q.

### Table 429 Q:

Table 429Q provides 54 compounds of formula (II-C) wherein G¹ is oxygen, R¹ is hydrogen, R² is G.081, X¹, X², X³ and X4 have the values listed in the table Q.

### Table 430 Q:

Table 430Q provides 54 compounds of formula (II-C) wherein G¹ is oxygen, R¹ is hydrogen, R² is G.082, X¹, X², X³ and X4 have the values listed in the table Q.

### Table 431 Q:

Table 431Q provides 54 compounds of formula (II-C) wherein G¹ is oxygen, R¹ is hydrogen, R² is G.083, X¹, X², X³ and X4 have the values listed in the table Q.

### Table 432 Q:

Table 432Q provides 54 compounds of formula (II-C) wherein G¹ is oxygen, R¹ is hydrogen, R² is G.084, X¹, X², X³ and X4 have the values listed in the table Q.

### Table 433 Q:

Table 433Q provides 54 compounds of formula (II-C) wherein G¹ is oxygen, R¹ is hydrogen, R² is G.085, X¹, X², X³ and X4 have the values listed in the table Q.

### Table 434 Q:

Table 434Q provides 54 compounds of formula (II-C) wherein G¹ is oxygen, R¹ is hydrogen, R² is G.086, X¹, X², X³ and X4 have the values listed in the table Q.

### Table 435 Q:

Table 435Q provides 54 compounds of formula (II-C) wherein G¹ is oxygen, R¹ is hydrogen, R² is G.087, X¹, X², X³ and X4 have the values listed in the table Q.

### Table 436 Q:

Table 436Q provides 54 compounds of formula (II-C) wherein G¹ is oxygen, R¹ is hydrogen, R² is G.088, X¹, X², X³ and X4 have the values listed in the table Q.

### Table 437 Q:

Table 437Q provides 54 compounds of formula (II-C) wherein G¹ is oxygen, R¹ is hydrogen, R² is G.089, X¹, X², X³ and X4 have the values listed in the table Q.

### Table 438 Q:

Table 438Q provides 54 compounds of formula (II-C) wherein G¹ is oxygen, R¹ is hydrogen, R² is G.090, X¹, X², X³ and X4 have the values listed in the table Q.

### Table 439 Q:

Table 439Q provides 54 compounds of formula (II-C) wherein G¹ is oxygen, R¹ is hydrogen, R² is G.091, X¹, X², X³ and X4 have the values listed in the table Q.

### Table 440 Q:

Table 440Q provides 54 compounds of formula (II-C) wherein G¹ is oxygen, R¹ is hydrogen, R² is G.092, X¹, X², X³ and X4 have the values listed in the table Q.

### Table 441 Q:

Table 441Q provides 54 compounds of formula (II-C) wherein G¹ is oxygen, R¹ is hydrogen, R² is G.093, X¹, X², X³ and X4 have the values listed in the table Q.

### Table 442 Q:

Table 442Q provides 54 compounds of formula (II-C) wherein G¹ is oxygen, R¹ is hydrogen, R² is G.094, X¹, X², X³ and X4 have the values listed in the table Q.

### Table 443 Q:

Table 443Q provides 54 compounds of formula (II-C) wherein G¹ is oxygen, R¹ is hydrogen, R² is G.095, X¹, X², X³ and X4 have the values listed in the table Q.

### Table 444 Q:

Table 444Q provides 54 compounds of formula (II-C) wherein G¹ is oxygen, R¹ is hydrogen, R² is G.096, X¹, X², X³ and X4 have the values listed in the table Q.

### Table 445 Q:

Table 445Q provides 54 compounds of formula (II-C) wherein G¹ is oxygen, R¹ is hydrogen, R² is G.097, X¹, X², X³ and X4 have the values listed in the table Q.

### Table 446 Q:

Table 446Q provides 54 compounds of formula (II-C) wherein G¹ is oxygen, R¹ is hydrogen, R² is G.098, X¹, X², X³ and X4 have the values listed in the table Q.

### Table 447 Q:

Table 447Q provides 54 compounds of formula (II-C) wherein G¹ is oxygen, R¹ is hydrogen, R² is G.099, X¹, X², X³ and X4 have the values listed in the table Q.

### Table 448 Q:

Table 448Q provides 54 compounds of formula (II-C) wherein G¹ is oxygen, R¹ is hydrogen, R² is G.100, X¹, X², X³ and X4 have the values listed in the table Q.

### Table 449 Q:

Table 449Q provides 54 compounds of formula (II-C) wherein G¹ is oxygen, R¹ is hydrogen, R² is G.101, X¹, X², X³ and X4 have the values listed in the table Q.

### Table 450 Q:

Table 450Q provides 54 compounds of formula (II-C) wherein G¹ is oxygen, R¹ is hydrogen, R² is G.102, X¹, X², X³ and X4 have the values listed in the table Q.

### Table 451 Q:

Table 451Q provides 54 compounds of formula (II-C) wherein G¹ is oxygen, R¹ is hydrogen, R² is G.103, X¹, X², X³ and X4 have the values listed in the table Q.

### Table 452 Q:

Table 452Q provides 54 compounds of formula (II-C) wherein G¹ is oxygen, R¹ is hydrogen, R² is G.104, X¹, X², X³ and X4 have the values listed in the table Q.

### Table 453 Q:

Table 453Q provides 54 compounds of formula (II-C) wherein G¹ is oxygen, R¹ is hydrogen, R² is G.105, X¹, X², X³ and X4 have the values listed in the table Q.

### Table 454 Q:

Table 4543Q provides 54 compounds of formula (II-C) wherein G¹ is oxygen, R¹ is hydrogen, R² is G.106, X¹, X², X³ and X4 have the values listed in the table Q.

### Table 455 Q:

Table 455Q provides 54 compounds of formula (II-C) wherein G¹ is oxygen, R¹ is hydrogen, R² is G.107, X¹, X², X³ and X4 have the values listed in the table Q.

### Table 456 Q:

Table 456Q provides 54 compounds of formula (II-C) wherein G¹ is oxygen, R¹ is hydrogen, R² is G.108, X¹, X², X³ and X4 have the values listed in the table Q.

### Table 457 Q:

Table 457Q provides 54 compounds of formula (II-C) wherein G¹ is oxygen, R¹ is hydrogen, R² is G.109, X¹, X², X³ and X4 have the values listed in the table Q.

### Table 458 Q:

Table 458Q provides 54 compounds of formula (II-C) wherein G¹ is oxygen, R¹ is hydrogen, R² is G.110, X¹, X², X³ and X4 have the values listed in the table Q.

### Table 459 Q:

Table 459Q provides 54 compounds of formula (II-C) wherein G¹ is oxygen, R¹ is hydrogen, R² is G.111, X¹, X², X³ and X4 have the values listed in the table Q.

### Table 460 Q:

Table 460Q provides 54 compounds of formula (II-C) wherein G¹ is oxygen, R¹ is hydrogen, R² is G.112, X¹, X², X³ and X4 have the values listed in the table Q.

### Table 461 Q:

Table 461Q provides 54 compounds of formula (II-C) wherein G¹ is oxygen, R¹ is hydrogen, R² is G.113, X¹, X², X³ and X4 have the values listed in the table Q.

### Table 462 Q:

Table 462Q provides 54 compounds of formula (II-C) wherein G¹ is oxygen, R¹ is hydrogen, R² is G.114, X¹, X², X³ and X4 have the values listed in the table Q.

### Table 463 Q:

Table 463Q provides 54 compounds of formula (II-C) wherein G¹ is oxygen, R¹ is hydrogen, R² is G.115, X¹, X², X³ and X4 have the values listed in the table Q.

### Table 464 Q:

Table 464Q provides 54 compounds of formula (II-C) wherein G¹ is oxygen, R¹ is hydrogen, R² is G.116, X¹, X², X³ and X4 have the values listed in the table Q.

| Het | Chemical structure |
|---|---|
| Het.01 | 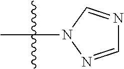 [SMILES: *n1cncn1] |
| Het.02 | 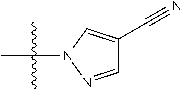 [SMILES: *n1cc(C#N)cn1] |
| Het.03 | 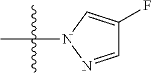 [SMILES: *n1cc(F)cn1] |
| Het.04 | 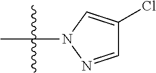 [SMILES: *n1cc(Cl)cn1] |

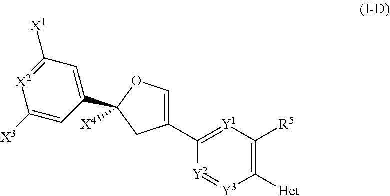

### Table 233P

Table 233P provides 3240 compounds of formula (I-D) wherein Het is Het.01, X¹, X², X³, Y¹, Y², Y³, X⁴ and R⁵ have the values listed in the table P.

### Table 234P

Table 234P provides 3240 compounds of formula (I-D) wherein Het is Het.02, X¹, X², X³, Y¹, Y², Y³, X⁴ and R⁵ have the values listed in the table P.

### Table 235 P

Table 235P provides 3240 compounds of formula (I-D) wherein Het is Het.03, X¹, X², X³, Y¹, Y², Y³, X⁴ and R⁵ have the values listed in the table P.

### Table 236 P

Table 236P provides 3240 compounds of formula (I-D) wherein Het is Het.04, X¹, X², X³, Y¹, Y², Y³, X⁴ and R⁵ have the values listed in the table P.

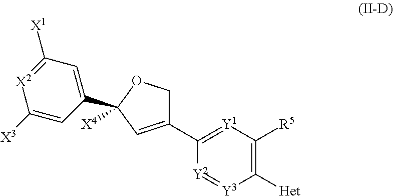

### Table 237 P

Table 237P provides 3240 compounds of formula (II-D) wherein Het is Het.01, X¹, X², X³, Y¹, Y², Y³, X⁴ and R⁵ have the values listed in the table P.

### Table 238 P

Table 238P provides 3240 compounds of formula (II-D) wherein Het is Het.02, X¹, X², X³, Y¹, Y², Y³, X⁴ and R⁵ have the values listed in the table P.

### Table 239 P

Table 239P provides 3240 compounds of formula (II-D) wherein Het is Het.03, X¹, X², X³, Y¹, Y², Y³, X⁴ and R⁵ have the values listed in the table P.

### Table 240 P

Table 240P provides 3240 compounds of formula (II-D) wherein Het is Het.04, X¹, X², X³, Y¹, Y², Y³, X⁴ and R⁵ have the values listed in the table P.

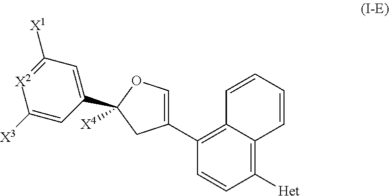

### Table 465Q

Table 465Q provides 54 compounds of formula (I-E) wherein G¹ is oxygen, R¹ is hydrogen, Het is Het.01, X¹, X², X³ and X4 have the values listed in the table Q.

### Table 466Q

Table 466Q provides 54 compounds of formula (I-E) wherein G¹ is oxygen, R¹ is hydrogen, Het is Het.02, X¹, X², X³ and X4 have the values listed in the table Q.

### Table 467Q

Table 467Q provides 54 compounds of formula (I-E) wherein G¹ is oxygen, R¹ is hydrogen, Het is Het.03, X¹, X², X³ and X4 have the values listed in the table Q.

### Table 468Q

Table 468Q provides 54 compounds of formula (I-E) wherein G¹ is oxygen, R¹ is hydrogen, Het is Het.04, X¹, X², X³ and X4 have the values listed in the table Q.

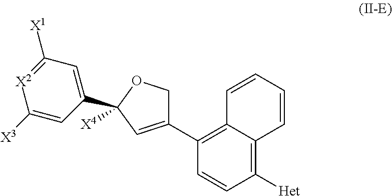

### Table 469Q

Table 469Q provides 54 compounds of formula (II-E) wherein G¹ is oxygen, R¹ is hydrogen, Het is Het.01, X¹, X², X³ and X4 have the values listed in the table Q.

### Table 470Q

Table 470Q provides 54 compounds of formula (II-E) wherein G¹ is oxygen, R¹ is hydrogen, Het is Het.02, X¹, X², X³ and X4 have the values listed in the table Q.

### Table 471Q

Table 471Q provides 54 compounds of formula (II-E) wherein G¹ is oxygen, R¹ is hydrogen, Het is Het.03, X¹, X², X³ and X4 have the values listed in the table Q.

### Table 472Q

Table 472Q provides 54 compounds of formula (II-E) wherein G¹ is oxygen, R¹ is hydrogen, Het is Het.04, X¹, X², X³ and X4 have the values listed in the table Q.

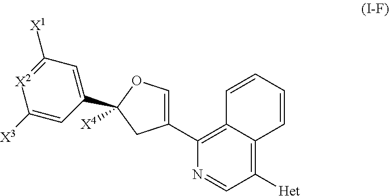

### Table 473Q

Table 473Q provides 54 compounds of formula (I-F) wherein G¹ is oxygen, R¹ is hydrogen, Het is Het.01, X¹, X², X³ and X4 have the values listed in the table Q.

### Table 474Q

Table 474Q provides 54 compounds of formula (I-F) wherein G¹ is oxygen, R¹ is hydrogen, Het is Het.02, X¹, X², X³ and X4 have the values listed in the table Q.

### Table 475Q

Table 475Q provides 54 compounds of formula (I-F) wherein G¹ is oxygen, R¹ is hydrogen, Het is Het.03, X¹, X², X³ and X4 have the values listed in the table Q.

### Table 476Q

Table 476Q provides 54 compounds of formula (I-F) wherein G¹ is oxygen, R¹ is hydrogen, Het is Het.04, X¹, X², X³ and X4 have the values listed in the table Q.

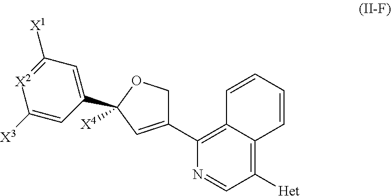

### Table 477Q

Table 477Q provides 54 compounds of formula (II-F) wherein G¹ is oxygen, R¹ is hydrogen, Het is Het.01, X¹, X², X³ and X4 have the values listed in the table Q.

### Table 478Q

Table 478Q provides 54 compounds of formula (II-F) wherein G¹ is oxygen, R¹ is hydrogen, Het is Het.02, X¹, X², X³ and X4 have the values listed in the table Q.

### Table 479Q

Table 479Q provides 54 compounds of formula (II-F) wherein G¹ is oxygen, R¹ is hydrogen, Het is Het.03, X¹, X², X³ and X4 have the values listed in the table Q.

### Table 480Q

Table 480Q provides 54 compounds of formula (II-F) wherein G¹ is oxygen, R¹ is hydrogen, Het is Het.04, X¹, X², X³ and X4 have the values listed in the table Q.

In one embodiment the invention provides a compound selected from Tables 1P to 116P, 1Q to 116Q, 233Q to 348Q, 233P to 236P 465Q to 468Q, 473Q to 476Q for use in controlling and/or preventing insects of the family Curculionidae, preferably in for use in controlling and/or preventing Anthonomus grandis.

Additional examples of insects from the family of Curculionidae are Anthonomus corvulus, Anthonomus elutus, Anthonomus elongatus, Anthonomus eugenii, Anthonomus consors, Anthonomus haematopus, Anthonomus lecontei, Anthonomus molochinus, Anthonomus morticinus, Anthonomus musculus, Anthonomus nigrinus, Anthonomus phyllocola, Anthonomus pictus, Anthonomus pomorum, Anthonomus quadrigibbus, Anthonomus rectirostris, Anthonomus rubi, Anthonomus santacruzi, Anthonomus signatus, Anthonomus subfasciatus, and Anthonomus tenebrosus.

In one embodiment the invention provides a compound selected from Tables 1P to 116P, 1Q to 116Q, 233Q to 348Q, 233P to 236P, 465Q to 468Q, 473Q to 476Q for use against Anthonomus grandis in cotton.

In one embodiment the invention provides a compound selected from Tables 1P to 116P, 1Q to 116Q, 233Q to 348Q, 233P to 236P, 465Q to 468Q, 473Q to 476Q for use in controlling and/or preventing soil pests.

In one embodiment the invention provides a compound selected from Tables 1P to 116P, 1Q to 116Q, 233Q to 348Q, 233P to 236P, 465Q to 468Q, 473Q to 476Q for use in controlling and/or preventing corn rootworm, in particular for use against corn root worm from the genus Diabrotica.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Diabrotica virgifera.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Diabrotica barberi.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Diabrotica undecimpunctata howardi.

In one embodiment the invention provides a compound selected from Tables 1P to 116P, 1Q to 116Q, 233Q to 348Q, 233P to 236P, 465Q to 468Q, 473Q to 476Q for use in controlling and/or preventing wireworms, in particular Agriotes spp.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Agriotes spp. in cereals, potato or corn.

Additional examples of Agriotes spp. include Agriotes lineatus, Agriotes obscurus, Agriotes brevis, Agriotes gurgistanus, Agriotes sputator, Agriotes ustulatus, Ctenicera destructor, and Limonius californicus.

In one embodiment the invention provides a compound selected from Tables 1P to 116P, 1Q to 116Q, 233Q to 348Q, 233P to 236P, 465Q to 468Q, 473Q to 476Q for use in controlling and/or preventing grubs, in particular white grubs.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Phyllophaga spp., particularly on corn, soybean or cotton.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Diloboderus spp. particularly on corn, soybean or cotton.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Popillia japonica, particularly on corn, soybean or cotton.

Additional examples of white grubs include Phyllophaga anxia, Phyllophaga crinite, Phyllophaga subnitida, Diloboderus abderus.

In one embodiment the invention provides a compound selected from Tables 1P to 116P, 1Q to 116Q, 233Q to 348Q, 233P to 236P, 465Q to 468Q, 473Q to 476Q for use in controlling and/or preventing termites, e.g. on sugarcane.

Examples of termites include Reticulitermes, Coptotermes, Macrotermes, Microtermes, Globitermes. Specific of subterranean termites include Reticulitermes flavipes, Reticulitermes hesperus, Reticulitermes verginicus, Reticulitermes hageni, Reticulitermes speratus, Reticulitermes lucifugus, Heterotermes aureus, Coptotermes formosanus, Coptotermes acinaciformis, Coptotermes curvignathus, Nasutitermes exitiosus, Nasutitermes walkeri, Mastotermes darwiniensis, Schedorhinotermes spp, Macrotermes bellicosus, Macrotermes spp., Globitermes sulphureus, Odontotermes spp. Specific examples of dry wood termites include Incisitermes minor, Marginitermes hubbardi, Cryptotermes brevis, Kalotermes flavicollis. Additional examples of termites include procornitermes spp. and procornitermes araujoi

In one embodiment the invention provides a compound selected from Tables 1P to 116P, 1Q to 116Q, 233Q to 348Q, 233P to 236P, 465Q to 468Q, 473Q to 476Q for use in controlling and/or preventing subterraneous stinkbugs, e.g. Scaptocoris spp.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Scaptocoris castaneus, in particular on cereals, soybean or corn.

In one embodiment the invention provides a compound selected from Tables 1P to 116P, 1Q to 116Q, 233Q to 348Q, 233P to 236P, 465Q to 468Q, 473Q to 476Q for use in controlling and/or preventing cutworms, e.g. agrotis spp.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Agrotis ipsilon, particularly on cereals, canola, soybean or corn.

In one embodiment the invention provides a compound selected from Tables 1P to 116P, 1Q to 116Q, 233Q to 348Q, 233P to 236P, 465Q to 468Q, 473Q to 476Q for use in controlling and/or preventing millipedes, e.g. Julus spp.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Julus spp., particularly on cereals, canola, soybean & corn.

In one embodiment the invention provides a compound selected from Tables 1P to 116P, 1Q to 116Q, 233Q to 348Q, 233P to 236P, 465Q to 468Q, 473Q to 476Q for use in controlling and/or preventing broca gigante, e.g. Telchin licus, particularly on sugarcane.

In one embodiment the invention provides a compound selected from Tables 1P to 116P, 1Q to 116Q, 233Q to 348Q, 233P to 236P, 465Q to 468Q, 473Q to 476Q for use in controlling and/or preventing whitefly.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Bemisia tabaci, particularly on vegetables, cotton, soybean, or potatoes.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Trialeurodes vaporariorum, particularly on vegetables, cotton, soybean, or potatoes.

In one embodiment the invention provides a compound selected from Tables 1P to 116P, 1Q to 116Q, 233Q to 348Q, 233P to 236P, 465Q to 468Q, 473Q to 476Q for use in controlling and/or preventing stinkbugs, in particular Euschistus spp.

In one embodiment the invention provides a compound selected from Tables 1P to 116P, 1Q to 116Q, 233Q to 348Q, 233P to 236P, 465Q to 468Q, 473Q to 476Q for use in controlling and/or preventing Euschistus spp., particularly in soybean.

Examples of stinkbugs include Nezara spp. (e.g. Nezara viridula, Nezara antennata, Nezara hilare), Piezodorus spp. (e.g. Piezodorus guildinii), Acrosternum spp. Euchistus spp. (e.g. Euchistus heros, Euschistus servus), Halyomorpha halys, Plautia crossota, Riptortus clavatus, Rhopalus msculatus, Antestiopsis orbitalus, Dichelops spp. (e.g. Dichelops furcatus, Dichelops melacanthus), Eurygaster spp. (e.g. Eurygaster intergriceps, Eurygaster maura), Oebalus spp. (e.g. Oebalus mexicana, Oebalus poecilus, Oebalus pugnase, Scotinophara spp. (e.g. Scotinophara lurida, Scotinophara coarctata). Preferred targets include Antestiopsis orbitalus, Dichelops furcatus, Dichelops melacanthus, Euchistus heros, Euschistus servus, Nezara viridula, Nezara hilare, Piezodorus guildinii, Halyomorpha halys. In one embodiment the stinkbug target is Nezara viridula, Piezodorus spp., Acrosternum spp, Euchistus heros.

In one embodiment the invention provides a compound selected from Tables 1P to 116P, 1Q to 116Q, 233Q to 348Q, 233P to 236P, 465Q to 468Q, 473Q to 476Q for use against rice pests.

In one embodiment the invention provides a compound selected from Tables 1P to 116P, 1Q to 116Q, 233Q to 348Q, 233P to 236P, 465Q to 468Q, 473Q to 476Q for use against stemborer, particularly in rice.

Examples of stemborers include Chilo sp, Chilo suppressalis, Chilo polychrysus, Chilo auricilius, Scirpophaga spp., Scirpophaga incertulas, Scirpophaga innotata, Scirpophaga nivella Sesamia sp, Sesamia inferens.

In one embodiment the invention provides a compound selected from Tables 1P to 116P, 1Q to 116Q, 233Q to 348Q, 233P to 236P, 465Q to 468Q, 473Q to 476Q for use against leaffolder, particularly in rice.

Examples of leaffolders include Cnaphalocrocis spp., Cnaphalocrocis medinalis, Marasmia spp., Marasmia patnalis, Marasmia exigua.

In one embodiment the invention provides a compound selected from Tables 1P to 116P, 1Q to 116Q, 233Q to 348Q, 233P to 236P, 465Q to 468Q, 473Q to 476Q for use against hoppers, particularly in rice.

Examples of Hoppers include Nephotettix spp., Nephotettix virescens, Nephotettix nigropictus, Nephotettix malayanus, Nephotettix cincticeps, Nilaparvata lugens, Sogatella furcifera.

In one embodiment the invention provides a compound selected from Tables 1P to 116P, 1Q to 116Q, 233Q to 348Q, 233P to 236P, 465Q to 468Q, 473Q to 476Q for use against gallmidge, particularly in rice.

Examples of Gall midge include Orseolia sp, Orseolia oryzae.

In one embodiment the invention provides a compound selected from Tables 1P to 116P, 1Q to 116Q, 233Q to 348Q, 233P to 236P, 465Q to 468Q, 473Q to 476Q for use against whorl maggot, particularly in rice.

Examples of whorl maggots include Hydrellia sp, Hydrellia philippina.

In one embodiment the invention provides a compound selected from Tables 1P to 116P, 1Q to 116Q, 233Q to 348Q, 233P to 236P, 465Q to 468Q, 473Q to 476Q for use against Rice bugs, particularly in rice.

Examples of rice bugs include Leptocorisa sp, Leptocorisa oratorius, Leptocorisa chinensis, Leptocorisa acuta.

In one embodiment the invention provides a compound selected from Tables 1P to 116P, 1Q to 116Q, 233Q to 348Q, 233P to 236P, 465Q to 468Q, 473Q to 476Q for use against Black bugs, particularly in rice.

Examples of Black bugs include Scotinophara sp, Scotinophara coarctata, Scotinophara lurida, Scotinophara latiuscula.

In one embodiment the invention provides a compound selected from Tables 1P to 116P, 1Q to 116Q, 233Q to 348Q, 233P to 236P, 465Q to 468Q, 473Q to 476Q for use against plutella spp.

In one embodiment the invention provides a compound selected from Tables 1P to 116P, 1Q to 116Q, 233Q to 348Q, 233P to 236P, 465Q to 468Q, 473Q to 476Q for use against Plutella xylostella, particularly in brassica crops.

In one embodiment the invention provides a compound selected from Tables 117P to 232P, 117Q to 232Q, 349Q to 464Q, 237P to 240P, 469Q to 472Q, 477Q to 480Q for use in controlling and/or preventing insects of the family Curculionidae, preferably in for use in controlling and/or preventing Anthonomus grandis.

Additional examples of insects from the family of Curculionidae are Anthonomus corvulus, Anthonomus elutus, Anthonomus elongatus, Anthonomus eugenii, Anthonomus consors, Anthonomus haematopus, Anthonomus lecontei, Anthonomus molochinus, Anthonomus morticinus, Anthonomus musculus, Anthonomus nigrinus, Anthonomus phyllocola, Anthonomus pictus, Anthonomus pomorum, Anthonomus quadrigibbus, Anthonomus rectirostris, Anthonomus rubi, Anthonomus santacruzi, Anthonomus signatus, Anthonomus subfasciatus, and Anthonomus tenebrosus.

In one embodiment the invention provides a compound selected from Tables 117P to 232P, 117Q to 232Q, 349Q to 464Q, 237P to 240P, 469Q to 472Q, 477Q to 480Q for use against Anthonomus grandis in cotton.

In one embodiment the invention provides a compound selected from Tables 117P to 232P, 117Q to 232Q, 349Q to 464Q, 237P to 240P, 469Q to 472Q, 477Q to 480Q for use in controlling and/or preventing soil pests.

In one embodiment the invention provides a compound selected from Tables 117P to 232P, 117Q to 232Q, 349Q to 464Q, 237P to 240P, 469Q to 472Q, 477Q to 480Q for use in controlling and/or preventing corn rootworm, in particular for use against corn root worm from the genus Diabrotica.

In one embodiment the invention provides a compound selected from Tables 117P to 232P, 117Q to 232Q, 349Q to 464Q, 237P to 240P, 469Q to 472Q, 477Q to 480Q for use in controlling and/or preventing corn Diabrotica virgifera.

In one embodiment the invention provides a compound selected from Tables 117P to 232P, 117Q to 232Q, 349Q to 464Q, 237P to 240P, 469Q to 472Q, 477Q to 480Q for use in controlling and/or preventing corn Diabrotica barberi.

In one embodiment the invention provides a compound selected from Tables 117P to 232P, 117Q to 232Q, 349Q to 464Q, 237P to 240P, 469Q to 472Q, 477Q to 480Q for use in controlling and/or preventing corn Diabrotica undecimpunctata howardi.

In one embodiment the invention provides a compound selected from Tables 117P to 232P, 117Q to 232Q, 349Q to 464Q, 237P to 240P, 469Q to 472Q, 477Q to 480Q for use in controlling and/or preventing wireworms, in particular Agriotes spp.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Agriotes spp. in cereals, potato or corn.

Additional examples of Agriotes spp. include Agriotes lineatus, Agriotes obscurus, Agriotes brevis, Agriotes gurgistanus, Agriotes sputator, Agriotes ustulatus, Ctenicera destructor, and Limonius californicus.

In one embodiment the invention provides a compound selected from Tables 117P to 232P, 117Q to 232Q, 349Q to 464Q, 237P to 240P, 469Q to 472Q, 477Q to 480Q for use in controlling and/or preventing grubs, in particular white grubs.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Phyllophaga spp., particularly on corn, soybean or cotton.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Diloboderus spp. particularly on corn, soybean or cotton.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Popillia japonica, particularly on corn, soybean or cotton.

Additional examples of white grubs include Phyllophaga anxia, Phyllophaga crinite, Phyllophaga subnitida, Diloboderus abderus.

In one embodiment the invention provides a compound selected from Tables 117P to 232P, 117Q to 232Q, 349Q to 464Q, 237P to 240P, 469Q to 472Q, 477Q to 480Q for use in controlling and/or preventing termites, e.g. on sugarcane.

Examples of termites include Reticulitermes, Coptotermes, Macrotermes, Microtermes, Globitermes. Specific of subterranean termites include Reticulitermes flavipes, Reticulitermes hesperus, Reticulitermes verginicus, Reticulitermes hageni, Reticulitermes speratus, Reticulitermes lucifugus, Heterotermes aureus, Coptotermes formosanus, Coptotermes acinaciformis, Coptotermes curvignathus, Nasutitermes exitiosus, Nasutitermes walkeri, Mastotermes darwiniensis, Schedorhinotermes spp, Macrotermes bellicosus, Macrotermes spp., Globitermes sulphureus, Odontotermes spp. Specific examples of dry wood termites include Incisitermes minor, Marginitermes hubbardi, Cryptotermes brevis, Kalotermes flavicollis. Additional examples of termites include procornitermes spp. and procornitermes araujoi

In one embodiment the invention provides a compound selected from Tables 117P to 232P, 117Q to 232Q, 349Q to 464Q, 237P to 240P, 469Q to 472Q, 477Q to 480Q for use in controlling and/or preventing subterraneous stinkbugs, e.g. Scaptocoris spp.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Scaptocoris castaneus, in particular on cereals, soybean or corn.

In one embodiment the invention provides a compound selected from Tables 117P to 232P, 117Q to 232Q, 349Q to 464Q, 237P to 240P, 469Q to 472Q, 477Q to 480Q for use in controlling and/or preventing cutworms, e.g. agrotis spp.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Agrotis ipsilon, particularly on cereals, canola, soybean or corn.

In one embodiment the invention provides a compound selected from Tables 117P to 232P, 117Q to 232Q, 349Q to 464Q, 237P to 240P, 469Q to 472Q, 477Q to 480Q for use in controlling and/or preventing millipedes, e.g. Julus spp.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Julus spp., particularly on cereals, canola, soybean & corn.

In one embodiment the invention provides a compound selected from Tables 117P to 232P, 117Q to 232Q, 349Q to 464Q, 237P to 240P, 469Q to 472Q, 477Q to 480Q for use in controlling and/or preventing broca gigante, e.g. Telchin licus, particularly on sugarcane.

In one embodiment the invention provides a compound selected from Tables 117P to 232P, 117Q to 232Q, 349Q to 464Q, 237P to 240P, 469Q to 472Q, 477Q to 480Q for use in controlling and/or preventing whitefly.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Bemisia tabaci, particularly on vegetables, cotton, soybean, or potatoes.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Trialeurodes vaporariorum, particularly on vegetables, cotton, soybean, or potatoes.

In one embodiment the invention provides a compound selected from Tables 117P to 232P, 117Q to 232Q, 349Q to 464Q, 237P to 240P, 469Q to 472Q, 477Q to 480Q for use in controlling and/or preventing stinkbugs, in particular Euschistus spp.

In one embodiment the invention provides a compound selected from Tables 117P to 232P, 117Q to 232Q, 349Q to 464Q, 237P to 240P, 469Q to 472Q, 477Q to 480Q for use in controlling and/or preventing Euschistus spp., particularly in soybean.

Examples of stinkbugs include Nezara spp. (e.g. Nezara viridula, Nezara antennata, Nezara hilare), Piezodorus spp. (e.g. Piezodorus guildinii), Acrosternum spp. Euchistus spp. (e.g. Euchistus heros, Euschistus servus), Halyomorpha halys, Plautia crossota, Riptortus clavatus, Rhopalus msculatus, Antestiopsis orbitalus, Dichelops spp. (e.g. Dichelops furcatus, Dichelops melacanthus), Eurygaster spp. (e.g. Eurygaster intergriceps, Eurygaster maura), Oebalus spp. (e.g. Oebalus mexicana, Oebalus poecilus, Oebalus pugnase, Scotinophara spp. (e.g. Scotinophara lurida, Scotinophara coarctata). Preferred targets include Antestiopsis orbitalus, Dichelops furcatus, Dichelops melacanthus, Euchistus heros, Euschistus servus, Nezara viridula, Nezara hilare, Piezodorus guildinii, Halyomorpha halys. In one embodiment the stinkbug target is Nezara viridula, Piezodorus spp., Acrosternum spp, Euchistus heros.

In one embodiment the invention provides a compound selected from Tables 117P to 232P, 117Q to 232Q, 349Q to 464Q, 237P to 240P, 469Q to 472Q, 477Q to 480Q for use against rice pests.

In one embodiment the invention provides a compound selected from Tables 117P to 232P, 117Q to 232Q, 349Q to 464Q, 237P to 240P, 469Q to 472Q, 477Q to 480Q for use against stemborer, particularly in rice.

Examples of stemborers include Chilo sp, Chilo suppressalis, Chilo polychrysus, Chilo auricilius, Scirpophaga spp., Scirpophaga incertulas, Scirpophaga innotata, Scirpophaga nivella Sesamia sp, Sesamia inferens.

In one embodiment the invention provides a compound selected from Tables 117P to 232P, 117Q to 232Q, 349Q to 464Q, 237P to 240P, 469Q to 472Q, 477Q to 480Q for use against leaffolder, particularly in rice.

Examples of leaffolders include Cnaphalocrocis spp., Cnaphalocrocis medinalis, Marasmia spp., Marasmia patnalis, Marasmia exigua.

In one embodiment the invention provides a compound selected from Tables 117P to 232P, 117Q to 232Q, 349Q to 464Q, 237P to 240P, 469Q to 472Q, 477Q to 480Q for use against hoppers, particularly in rice.

Examples of Hoppers include Nephotettix spp., Nephotettix virescens, Nephotettix nigropictus, Nephotettix malayanus, Nephotettix cincticeps, Nilaparvata lugens, Sogatella furcifera.

In one embodiment the invention provides a compound selected from Tables 117P to 232P, 117Q to 232Q, 349Q to 464Q, 237P to 240P, 469Q to 472Q, 477Q to 480Q for use against gallmidge, particularly in rice.

Examples of Gall midge include Orseolia sp, Orseolia oryzae.

In one embodiment the invention provides a compound selected from Tables 117P to 232P, 117Q to 232Q, 349Q to 464Q, 237P to 240P, 469Q to 472Q, 477Q to 480Q for use against whorl maggot, particularly in rice.

Examples of whorl maggots include Hydrellia sp, Hydrellia philippina.

In one embodiment the invention provides a compound selected from Tables 117P to 232P, 117Q to 232Q, 349Q to 464Q, 237P to 240P, 469Q to 472Q, 477Q to 480Q for use against Rice bugs, particularly in rice.

Examples of rice bugs include Leptocorisa sp, Leptocorisa oratorius, Leptocorisa chinensis, Leptocorisa acuta.

In one embodiment the invention provides a compound selected from Tables 117P to 232R 117Q to 232Q, 349Q to 464Q, 237P to 240P, 469Q to 472Q, 477Q to 480Q for use against Black bugs, particularly in rice.

Examples of Black bugs include Scotinophara sp, Scotinophara coarctata, Scotinophara lurida, Scotinophara latiuscula.

In one embodiment the invention provides a compound selected from Tables 117P to 232P, 117Q to 232Q, 349Q to 464Q, 237P to 240P, 469Q to 472Q, 477Q to 480Q for use against plutella spp.

In one embodiment the invention provides a compound selected from Tables 117P to 232P, 117Q to 232Q, 349Q to 464Q, 237P to 240P, 469Q to 472Q, 477Q to 480Q for use against Plutella xylostella, particularly in brassica crops.

The compounds of the invention may be made by a variety of methods as shown in the following Schemes.

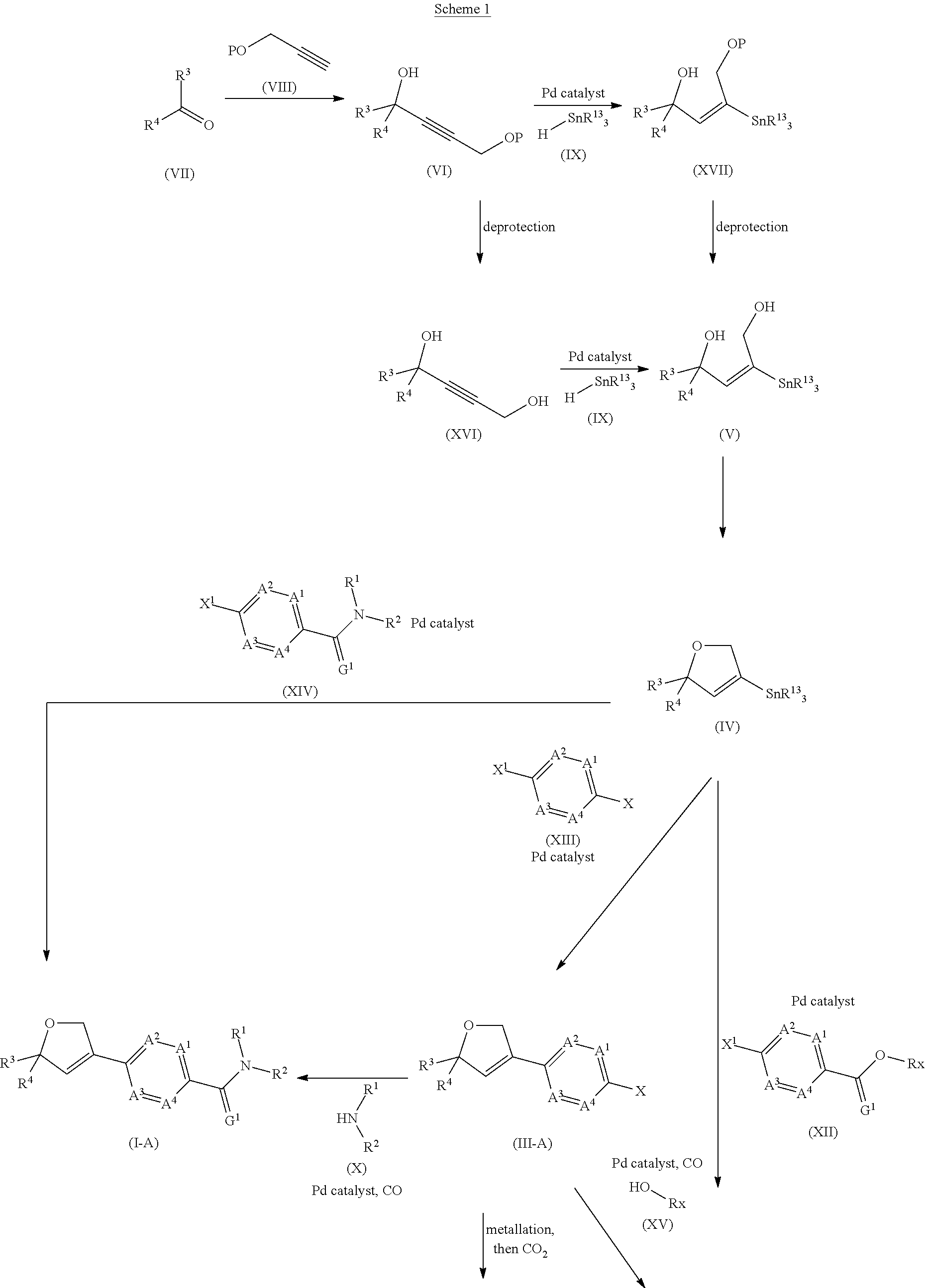

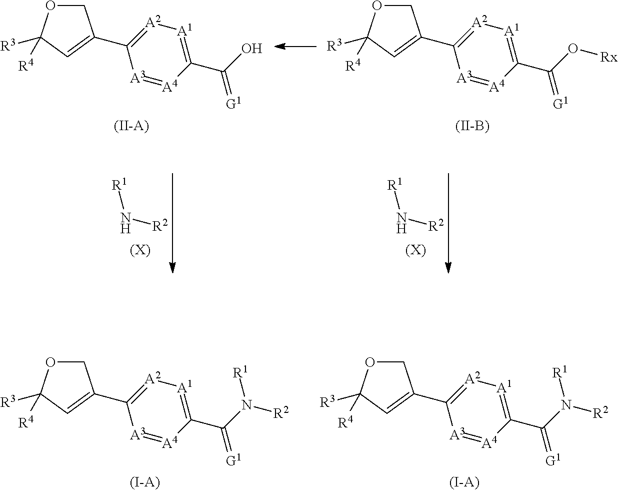

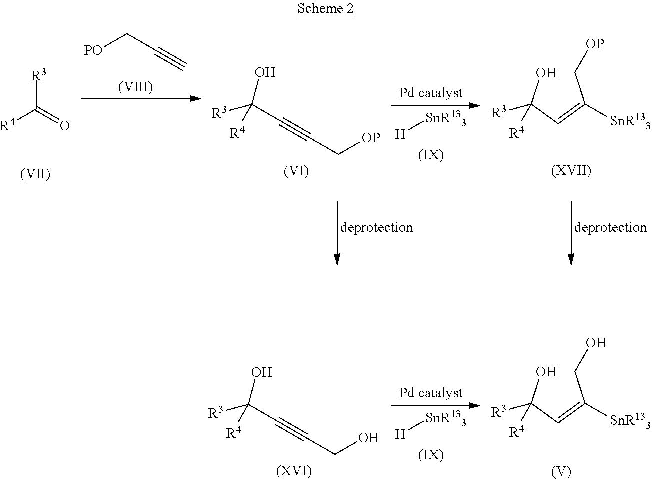

1) Compounds of formula (V), wherein R¹³ is C₁-C₆alkyl, can be prepared by deprotection of a compound of formula (XVII), wherein R¹³ is C₁-C₆alkyl and wherein P is a protecting group for an alcohol. Various protecting groups can be used, such as those described in T. W. Green, P. G. M. Wuts, Protective Groups in Organic Synthesis. Depending on the protecting group used, different methods are available to achieve the deprotection, as is also described in the previous reference. For instance, if such a protecting group is a trimethylsilyl group, these reactions are usually carried out in the presence of a suitable fluoride ion source, such as tetrabutylammonium fluoride optionally in a solvent, such as ethanol or tetrahydrofuran, or mixtures thereof. The reaction is carried out at a temperature of from 0° C. to 100° C., preferably from 0° C. to ambient temperature. The alcohol deprotection reactions are known in the literature and can be achieved using methods known to a person skilled in the art.
2) Compounds of formula (XVII), wherein R¹³ is C₁-C₆alkyl, can be prepared by reacting a compound of formula (VI) wherein P is a protecting group, for example a trimethylsilyl group, with a compound of formula (IX), wherein R¹³ is C₁-C₆alkyl, in the presence of a catalyst, such as tetrakis(triphenylphosphine)palladium or PdCl₂(PPh₃)₂. These reactions are usually carried out in the presence of a suitable solvent, such as toluene, dichloromethane or chlorobenzene. The reaction is carried out at a temperature of from −78° C. to 100° C., preferably from 0° C. to ambient temperature. Alternatively, the hydrostannylation reaction can be achieved under radical conditions or in the presence of copper salts. Stannylation of enynes are known in the literature (see references in Pancrazi and al. J. Org. Chem. 1997, 62, 7768-7780) and can be achieved using methods known to a person skilled in the art.
3) Compounds of formula (VI) can be prepared by reacting a compound of formula (VIII) wherein P is a protecting group, for example a trimethylsilyl group, with a compound of formula (VII), in a stepwise process. First a compound of formula (VIII) wherein P is a protecting group, for example a trimethylsilyl group, is treated with a metallating agent, such as butyl lithium or a Grignard reagent, such as ethyl magnesium bromide to form an intermediate that is then reacted with a compound of formula (VII). These reactions are usually carried out in the presence of a suitable solvent, such as toluene, dichloromethane or tetrahydrofuran. The reaction is carried out at a temperature of from −40° C. to 100° C., preferably from 0° C. to ambient temperature. The alkylation of ketones are known in the literature and can be achieved using methods known to a person skilled in the art.
4) Compounds of formula (V), wherein R¹³ is C₁-C₆alkyl, can be prepared from compounds of formula (XVI) using the method described in 2).
5) Compounds of formula (XVI) can be prepared from compounds of formula (VI) using the method described in 1).

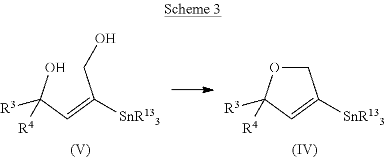

6) Compounds of formula (IV), wherein R¹³ is C₁-C₆alkyl, can be prepared by dehydration of a compound of formula (V) wherein R¹³ is C₁-C₆alkyl. Such reactions are usually carried out in the presence of an acid, for example an inorganic acid, such as hydrochloric acid or sulfuric acid, or a sulfonic acid, such as methanesulfonic acid, optionally in a solvent, such as water, ethanol or tetrahydrofuran, or mixtures thereof. The reaction is carried out at a temperature of from 0° C. to 100° C., preferably from 40° C. to 80° C. Alternatively, dehydration can be carried out using a dehydrating agent, such as phosphorus pentoxide, in a solvent, such as chloroform, at a temperature of from −20° C. to 50°, preferably at 0° C. Alternatively, cyclisation can be carried out under Mitsunobu conditions involving treatment of a compound of formula (V) with a phosphine, such as triphenylphosphine, and an azodicarboxylate reagent, such as diethyl azodicarboxylate, diisopropyl azodicarboxylate or dicyclohexyl azodicarboxylate, in a solvent, such as tetrahydrofuran, at a temperature of from 0° C. to 80° C., preferably from 0° C. to ambient temperature.

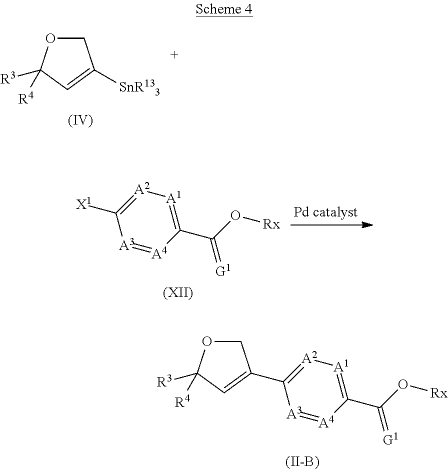

7) Compounds of formula (II-B) wherein Rx is C₁-C₁₅alkoxy can be prepared by reacting a compound of formula (XII) wherein X¹ is a leaving group, for example a halogen, such as bromo, and wherein Rx is C₁-C₁₅alkoxy with a compound of formula (IV), wherein R¹³ is C₁-C₆alkyl, in the presence of a catalyst, such as tetrakis(triphenylphosphine)palladium or PdCl₂(PPh₃)₂, a ligand, such as a triphenylphosphine, and an additive, such as lithium chloride, or copper iodide. The reaction is carried out in a suitable solvent, such as toluene, at a temperature of from 50° C. to 200° C., preferably from 100° C. to 150° C., in particular at 115° C. The Stille couplings are known in the literature and can be achieved using methods known to a person skilled in the art (see S. P. H. Mee, V. Lee, J. E. Baldwin, Angew. Chem. Int. Ed., 2004, 43, 1132-1136.)

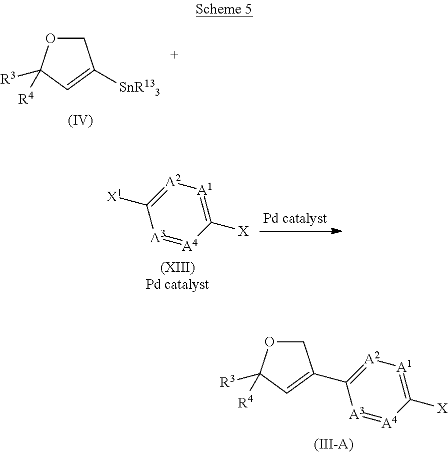

8) Compounds of formula (III-A) can be prepared by reacting a compound of formula (XIII) wherein X¹ and X are leaving groups, for example a halogen, such as bromo, with a compound of formula (IV), wherein R¹³ is C₁-C₆alkyl, using the same methods as described in 7).

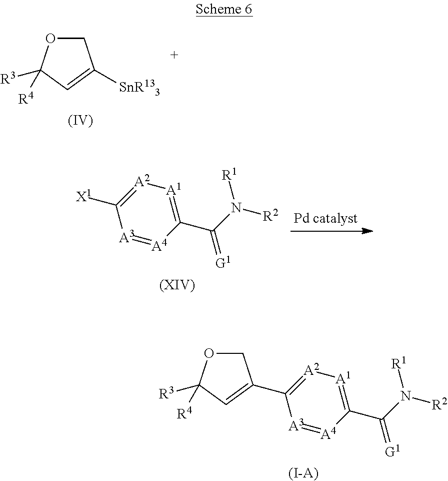

9) Compounds of formula (I-A) can be prepared by reacting a compound of formula (XIV) wherein X¹ is a leaving group, for example a halogen, such as bromo, with a compound of formula (IV), wherein R¹³ is C₁-C₆alkyl, using the same methods as described in 7).

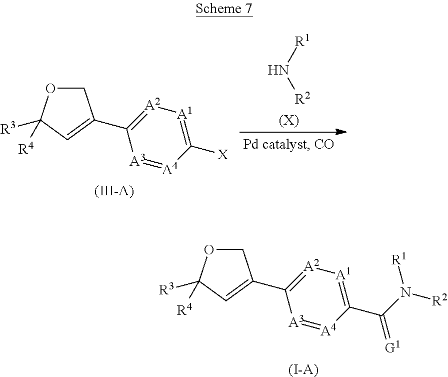

10) Compounds of formula (I-A) can be prepared by reacting a compound of formula (IIIA) wherein X is a leaving group, for example a halogen, such as bromo, with carbon monoxide and an amine of formula (X), in the presence of a catalyst, such as palladium(II) diacetate, a ligand, such as a phosphine ligand, such as tributylphosphine, and a base such as cesium carbonate, or diisopropylethylamine (Hunig's base). The reaction is carried out in a suitable solvent, such as toluene, at a temperature of from 50° C. to 200° C., preferably from 100° C. to 150° C., in particular at 115° C. The reaction is carried out at a pressure of from 50 to 200 bar, preferably from 100 to 150 bar, in particular at 120 bar.

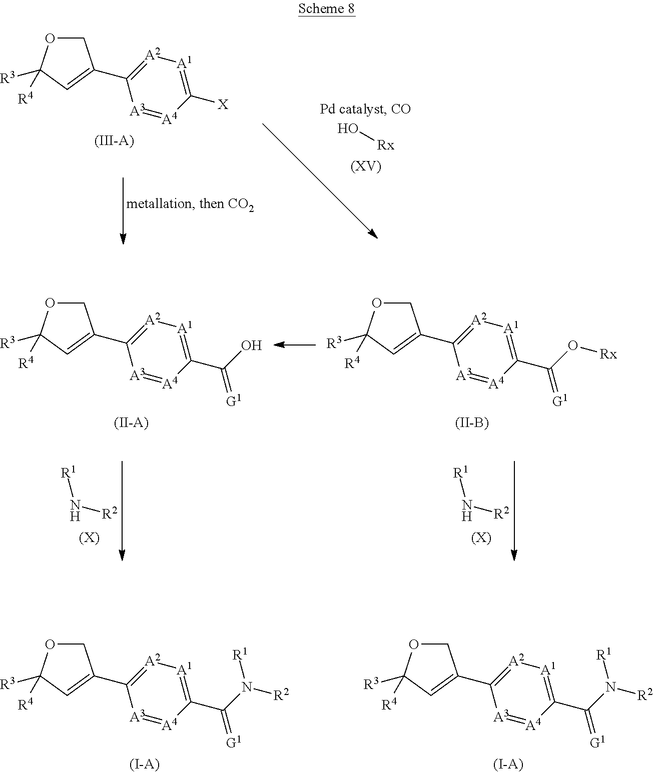

11) Compounds of formula (I-A) can be made by treatment of a compound of formula (II-A) with a compound of formula (X) and a dehydrating reagent. Alternatively, carboxylic acid (II-A) is transformed to an activated derivative, such as an acid chloride, for instance by treatment with thionyl chloride, or a mixed anhydride, for instance by treatment with ethyl chloroformate, and the activated derivative is reacted with a compound of formula (X), optionally in the presence of a base, and in a suitable solvent, such as, for instance, tetrahydrofuran. The reaction is carried out at a temperature of from −120° C. to +130° C., preferably from −100° C. to 100° C.
12) Alternatively, compounds of formula (I-A) can be made from a compound of formula (II-B) wherein Rx is C₁-C₁₅alkoxy by heating the ester and an amine of formula (X) together in a thermal process Amines of formula (X) are known in the literature or can be prepared using methods known to a person skilled in the art.
13) Compounds of formula (II-A), can be made by treatment of a compound of formula (III-A), wherein X is a halogen, for instance bromine, with a metallating agent, such as a metal, for instance magnesium, or an organometallic compound, for instance butyllithium, followed by the treatment with carbon dioxide. The reaction is carried out at a temperature of from −120° C. to +130° C., preferably from −100° C. to 100° C.
14) Compounds of formula (II-A) can be made by hydrolysis of a compound of formula (II-B), and Rₓ is C₁-C₁₅alkyl, such as methyl or tert-butyl. For instance, in the case where Rₓ is methyl or ethyl, the hydrolysis can be done with water and a base, such as potassium hydroxide, in the absence or in the presence of a solvent, such as, for instance, tetrahydrofuran or methynol. In the case where Rx is, for example, tert-butyl, the hydrolysis is done in the presence of acid, such as trifluoroacetic acid or hydrochloric acid. The reaction is carried out at a temperature of from −120° C. to +130° C., preferably from −100° C. to 100° C.
15) Compounds of formula (II-B) wherein Rx is C₁-C₁₅alkoxy, can be prepared by reacting a compound of formula (III-A) wherein X is a leaving group, for example a halogen, such as bromo, with carbon monoxide and an alcohol of formula Rx-OH, such as ethanol, in the presence of a catalyst, such as bis(triphenylphosphine)palladium(II) dichloride, and a base, such as pyridine, triethylamine, 4-(dimethylamino)-pyridine (“DMAP”) or diisopropylethylamine (Hunig's base). The reaction is carried out at a temperature of from 50° C. to 200° C., preferably from 100° C. to 150° C., in particular at 115° C. The reaction is carried out at a pressure of from 50 to 200 bar, preferably from 100 to 150 bar, in particular at 120 bar.

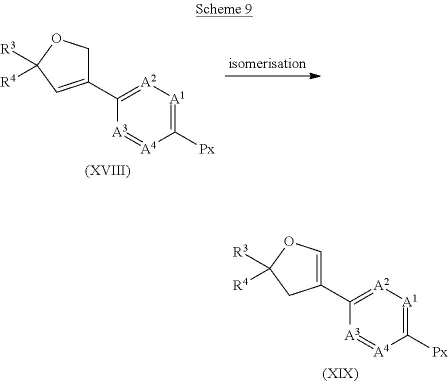

16) 2,3-Dihydrofuran compounds of formula (XIX) wherein Px is P as defined in the claims (an amide or heterocycle), a leaving group for example a halogen, such as bromo, or C(O)R wherein R is halogen, OH or C₁-C₁₅alkoxy, may be prepared by isomerisation of 2,5-dihydrofuran of formula (XVIII) wherein Px is P as defined in the claims, a leaving group for example a halogen, such as bromo, or C(O)R wherein R is halogen, OH or C₁-C₁₅alkoxy, using a metal catalyst such as RhCl(PPh₃)₃, RhH(PPh₃)₄, H₂Ru(CO)(PPh₃)₃, HClRu(CO)(PPh₃)₃ or H₂Ru(PPh₃)₄ in a solvent such as toluene or an alcoholic solvent such as ethanol at a temperature of between room temperature and 150° C., preferably between 80° C. and 120° C. Such conditions of isomerisation of 2,5-dihydrofuran compounds have been described in Chem. Eur. J. 2003, 9, 4442-4451 using the general catalytic isomerisation described by M. Mori et al in J. Org. Chem. 2000, 65, 3966-3970 or M. Bartok et al in J. Organomet. Chem. 1985, 297, C37-C40. Alternatively, the isomerisation may be performed in the presence of basic oxide metal catalysts such as MgO, CaO, SrO, or La₂O₃ as described by K. Tanabe in Chem. Lett. 1981, 341-342 for the isomerisation of 2,5-dihydrofuran.

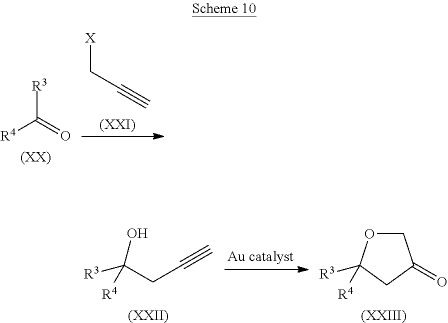

17) Compounds of formula (XXIII) can be prepared by hydrative cyclisation of a compound of formula (XXII) These reactions are usually carried out in the presence of a suitable lewis acid, such as a gold catalyst, as described in J. Am. Chem. Soc., 2010, 132 (10), pp 3258-3259. The reaction is usually carried out using (Triphenylphosphine)gold(I) bis(trifluoromethanesulfonyl)imidate, in the presence of a pyridine N-oxyde, such as 5-Bromo-1-oxy-nicotinic acid methyl ester and an acid, such as methanesulfonic acid, in an aprotic solvent, such as 1,2-dichloroethane. The reaction is carried out at a temperature of from 0° C. to 100° C., preferably from 0° C. to 40° C.
18) Compounds of formula (XXII) can be prepared by reacting a compound of formula (XX) with a compound of formula (XXI), where X is a halogen. These reactions are usually carried out in the presence of a metal, such as magnesium, lithium, indium, cerium or zinc, in a suitable solvent, such as tetrahydrofuran, diethyl ether or N,N-dimethylformamide. The reaction is carried out at a temperature of from −78° C. to 100° C., preferably from −78° C. to ambient temperature. Alternatively, Compounds of formula (XXII) can be prepared by reacting a compound of formula (XX) with a compound of formula (XXI), where X is a trialkylsilyl group. These reactions are usually carried out in the presence of strong base, such as lithium diisopropylamide, in a suitable solvent, such as tetrahydrofuran, diethyl ether or N, N-dimethylformamide. The reaction is carried out at a temperature of from −78° C. to 100° C., preferably from −78° C. to ambient temperature.

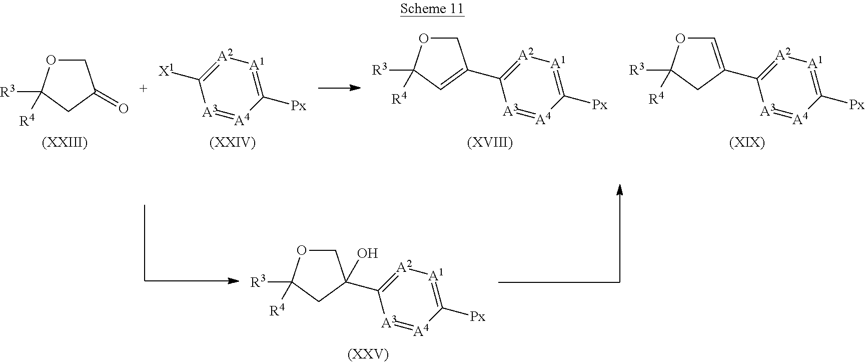

19) Compounds of formula (XVIII) and (XIX) wherein Px is P as defined in the claims, a leaving group for example a halogen, such as bromo, or C(O)R wherein R is halogen, OH or C₁-C₁₅alkoxy, can be prepared by reacting a compound of formula (XXIV) wherein X¹ is a leaving group, for example a halogen, such as iodo or bromo and wherein Px is P as defined in the claims, a leaving group for example a halogen, such as bromo, or C(O)R wherein R is halogen, OH or C₁-C₁₅alkoxy, with a compound of formula (XXIII), in the presence of a metal, such as catalyst, such as magnesium, lithium, indium, cerium or zinc, in a suitable solvent, such as tetrahydrofuran, diethyl ether or N,N-dimethylformamide. The reaction is carried out at a temperature of from −78° C. to 100° C., preferably from −78° C. to ambient temperature.
20) Compounds of formula (XXV) can be prepared by reacting a compound of formula (XXIV) wherein X¹ is a leaving group, for example a halogen, such as iodo or bromo, and wherein Px is P as defined in the claims, a leaving group for example a halogen, such as bromo, or C(O)R wherein R is halogen, OH or C₁-C₁₅alkoxy, with a compound of formula (XXIII), in the presence of a metal, such as magnesium, indium, cerium, zinc, or an organolithium reagent, such as n-butyl lithium, in a suitable solvent, such as tetrahydrofuran, diethyl ether or N,N-dimethylformamide. The reaction is carried out at a temperature of from −100° C. to 100° C., preferably from −100° C. to ambient temperature.
21) Compounds of formula (XVIII) and (XIX) wherein Px is P as defined in the claims, a leaving group for example a halogen, such as bromo, or C(O)R wherein R is halogen, OH or C₁-C₁₅alkoxy, can be prepared by reacting a compound of formula (XXV) wherein Px is P as defined in the claims, a leaving group for example a halogen, such as bromo, or C(O)R wherein R is halogen, OH or C₁-C₁₅alkoxy, in the presence of an acid, such as p-toluenesulfonic acid or sulphuric acid, or in the presence of a dehydrating agent, such as POCl₃ in a suitable solvent, such as tetrahydrofuran, diethyl ether or dichloromethane. The reaction is carried out at a temperature of from −78° C. to 100° C., preferably from −40° C. to ambient temperature.

Alternatively, compounds of formula (XVIII) and (XIX) wherein Px is P as defined in the claims, a leaving group for example a halogen, such as bromo, or C(O)R wherein R is halogen, OH or C₁-C₁₅alkoxy, can be prepared by reacting a compound of formula (XXV) wherein Px is P as defined in the claims, a leaving group for example a halogen, such as bromo, or C(O)R wherein R is halogen, OH or C₁-C₁₅alkoxy, in the presence of a chlorinating agent, such as thionyl chloride or oxalyl chloride, or an acetylating agent, such as acetic anhydride in the presence of a base, such as triethylamine, potassium carbonate or pyridine, in a suitable solvent, such as tetrahydrofuran, diethyl ether or dichloromethane. The reaction is carried out at a temperature of from −78° C. to 100° C., preferably from −40° C. to ambient temperature.

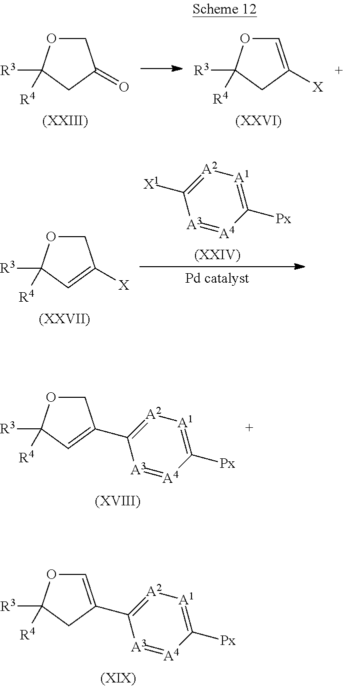

22) Compounds of formula (XVIII) (and compounds of formula (XIX)) wherein Px is P as defined in the claims, a leaving group for example a halogen, such as bromo, or C(O)R wherein R is halogen, OH or C₁-C₁₅alkoxy, can be prepared by reacting a compound of formula (XXVI) (and respectively compounds of formula (XXVII)) wherein X is a leaving group, for example a halogen, such as bromo, or a triflate, with a compound of formula (XXIV) wherein Px is P as defined in the claims, a leaving group for example a halogen, such as bromo, or C(O)R wherein R is halogen, OH or C₁-C₁₅alkoxy, wherein X¹ is a boron derivative, such as a boronic acid, a pinacolboronate, or a trifluoroborate salt, in a Suzuki coupling reaction, in the presence of a palladium catalyst, such as palladium acetate or tetrakis(triphenylphosphine) palladium, in a suitable solvent, such as 1,4-dioxane, touene, acetonitrile or N,N-dimethylformamide The reaction is carried out at a temperature of from −20° C. to 150° C., preferably from ambient temperature to 100° C. Alternatively, compounds of formula (XVIII) (and compounds of formula (XIX)) wherein Px is P as defined in the claims, a leaving group for example a halogen, such as bromo, or C(O)R wherein R is halogen, OH or C₁-C₁₅alkoxy, can be prepared by reacting a compound of formula (XXVI) (and respectively compounds of formula (XXVII)) wherein X is a leaving group, for example a halogen, such as bromo, or a triflate and wherein Px is P as defined in the claims, a leaving group for example a halogen, such as bromo, or C(O)R wherein R is halogen, OH or C₁-C₁₅alkoxy, with a compound of formula (XXIV) wherein X¹ is a trialkylstannane derivative, such as tributyltin, or respectively an organozinc derivative in a Stille or Negishi coupling reaction, in the presence of a palladium catalyst, such as palladium acetate or tetrakis(triphenylphosphine) palladium, in a suitable solvent, such as 1,4-dioxane, touene, acetonitrile or N,N-dimethylformamide
23) Compounds of formula (XXVI) (and compounds of formula (XXVII)) wherein X is a halogen, such as bromo, can be prepared by reacting a compound of formula (XXIII) with a brominating agent, such as phosphoric tribromide, in a suitable solvent, such as tetrahydrofuran, or chloroform, dichloromethane. The reaction is carried out at a temperature of from −40° C. to 100° C., preferably from −40° C. to ambient temperature. Alternatively, compounds of formula (XXVI) (and compounds of formula (XXVII)) wherein X is a triflate, can be prepared by reacting a compound of formula (XXIII) with a triflating agent, such as triflic anhydride or N,N-bis(trifluoromethanesulfonyl)aniline, in the presence of a base, such as 4-picoline, sodium or potassium hexamethyldisilylamide, lithium diisopropylamide, triethylamine or 2,6-lutidine in a suitable solvent, such as tetrahydrofuran, chloroform or dichloromethane. The reaction is carried out at a temperature of from −100° C. to 150° C., preferably from −40° C. to 100° C.

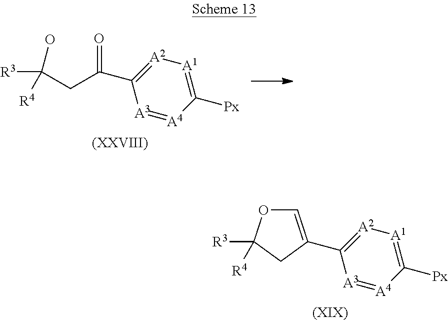

24) Compounds of formula (XIX) wherein Px is P as defined in the claims, a leaving group for example a halogen, such as bromo, or C(O)R wherein R is halogen, OH or C₁-C₁₅alkoxy, can be prepared by reacting a compound of formula (XXVIII) wherein Px is P as defined in the claims, a leaving group for example a halogen, such as bromo, or C(O)R wherein R is halogen, OH or C₁-C₁₅alkoxy, with trimethylsilyldiazomethane, in the presence of an organometallic reagent, such as methyl lithium, in a suitable solvent, such as tetrahydrofuran, diethyl ether, N,N-dimethylformamide or dimethoxyethane. The reaction is carried out at a temperature of from −78° C. to 100° C., preferably from −78° C. to ambient temperature.

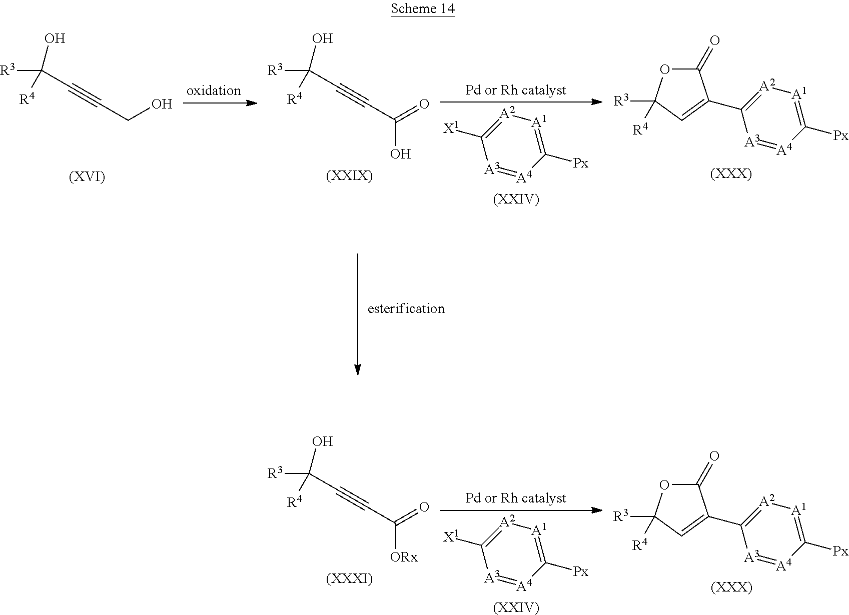

25) Compounds of formula (XXX) can be prepared by reaction a compound of formula (XXIV), wherein X¹ is an halogen, such as bromo or iodo, or a boron derivative, such as a boronic acid, a pinacol boronate or a trifluoroboronate salt and wherein Px is P as defined in the claims, a leaving group for example a halogen, such as bromo, or C(O)R wherein R is halogen, OH or C₁-C₁₅alkoxy, with a compound of formula (XXXI), wherein Rx is C₁-C₁₅alkoxy (or with compounds of formula (XXIX)). The reaction is usually carried out in the presence of a palladium or rhodium catalyst, such as palladium acetate, in the presence of a ligand, such as triphenylphosphine, tricyclohexylphosphine or tri(tert-butyl)phosphine, and of an acid, such as acetic acid, or formic acid. These reactions are usually carried out in the presence of a suitable solvent, such as toluene, dichloromethane or chlorobenzene. The reaction is carried out at a temperature of from −78° C. to 150° C., preferably from 0° C. to 100° C.
26) Compounds of formula (XXXI), wherein Rx is C₁-C₁₅alkoxy, can be made by transforming a compound of formula (XXIX) into an activated derivative, such as an acid chloride, for instance by treatment with thionyl chloride, or a mixed anhydride, for instance by treatment with ethyl chloroformate, and the activated derivative is reacted with a an alcohol, of formula RxOH, optionally in the presence of a base, and in a suitable solvent, such as, for instance, tetrahydrofuran. The reaction is carried out at a temperature of from −120° C. to +130° C., preferably from −100° C. to 100° C.
27) Compounds of formula (XXIX) can be made by oxidizing a compound of formula (XVI) into a carboxylic acid, using an oxidizing agent, such as a chromium derivative, and in a suitable solvent, such as, for instance, dichloromethane. The reaction is carried out at a temperature of from −120° C. to +130° C., preferably from −100° C. to 100° C. Such oxidations of alcohols into carboxylic acids are known in the literature and can be achieved using methods known to a person skilled in the art.

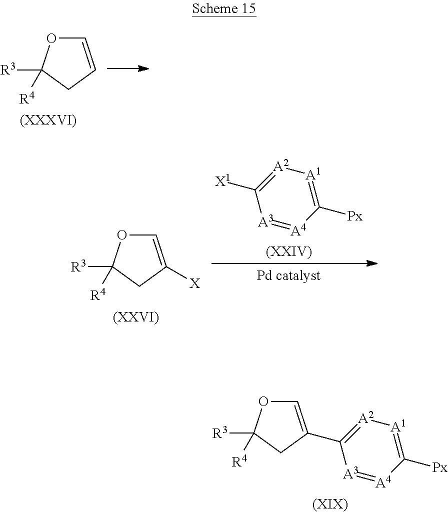

28) Compounds of formula (XXVI) when X is a boron derivative, e.g. boronic acid, a pinacolboronate or a trifluoroborate salt, can be prepared from compounds of formula (XXXVI) (e.g. prepared according to 47) or by ring closing metathesis, as in Journal of Organic Chemistry (2004), 69(22), 7672-7687, or using methods known by a person skilled in the art, such as in Chemistry—A European Journal, 7(11), 2349-2369; 2001) using a diboron derivative, such as pinacol diborane, in the presence of a suitable catalyst, such as iridium, in the presence of a suitable ligand, such as 4,4′-Di-tert-butylbipyridine as is described for example in Chemistry—An Asian Journal (2008), 3(12), 2082-2090.
29) Compounds of formula (XIX) can be prepared from compounds of formula (XXIV) and (XXVI) when X is a boron derivative and X¹ is a leaving group for example a halogen, such as bromo, or C(O)R wherein R is halogen, OH or C₁-C₁₅alkoxy, using a Suzuki reaction, e.g. as described in 22).
30) Compounds of formula (XXVI) when X is tin derivative, e.g. SnR¹³₃ wherein R¹³ is C₁-C₆alkyl, e.g. tributyltin, can be prepared from compounds of formula (XXXVI) using a trialkyltin hydride as is described for example in Journal of Organic Chemistry, 69(1), 72-78; 2004.
31) Compounds of formula (XIX) can be prepared from compounds of formula (XXIV) and (XXVI) when X is a tin derivative and X¹ is a leaving group, for example a halogen, such as bromo, using the methods described in 7).

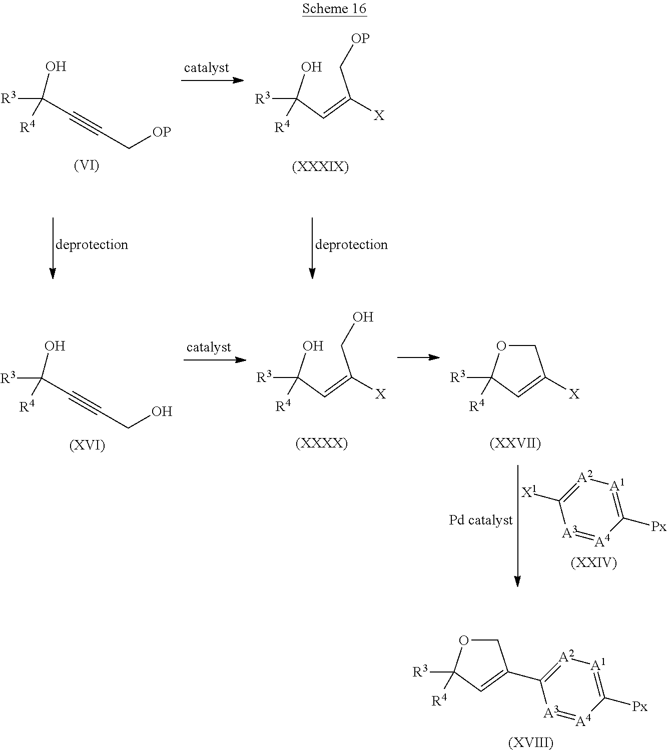

32) Compounds of formula (XXVII) when X is a boron derivative, e.g. boronic acid, a pinacolboronate or a trifluoroborate salt, can be prepared from compounds of formula (XXXX) as is described in 6).
33) Compounds of formula (XVIII) can be prepared from compounds of formula (XXIV) and (XXVII) when X is a boron derivative and X¹ is a leaving group for example a halogen, such as bromo, or C(O)R wherein R is halogen, OH or C₁-C₁₅alkoxy, using a Suzuki reaction, e.g. as described in 22).
34) Compounds of formula (XXXX) when X is a boron derivative, e.g. boronic acid, a pinacolboronate or a trifluoroborate salt, can be prepared by treating a compounds of formula (XVI) with a borane derivative, such as pinacol borane or catecholborane in a suitable solvent such as tetrahydrofuran or dichloromethane. Alternatively, Compounds of formula (XXXX) when X is a boron derivative, e.g. boronic acid, a pinacolboronate or a trifluoroborate salt, can be prepared by treating a compounds of formula (XVI) with a diboron derivative, such as pinacol diborane, in the presence of a suitable catalyst, such as copper, palladium of nickel, as is described for example in Chemical Communications, 2008, 6, 733-734 and Angewandte Chemie, International Edition, 47(52), 10183-10186; 2008. Methodology suitable for the preparation of compounds of formula (XXXX) when X is a boron derivative, e.g. boronic acid, a pinacolboronate or a trifluoroborate salt, from compounds of formula (XXXIX) when X is a boron derivative, e.g. boronic acid, a pinacolboronate or a trifluoroborate salt, is described in Schemes 1)

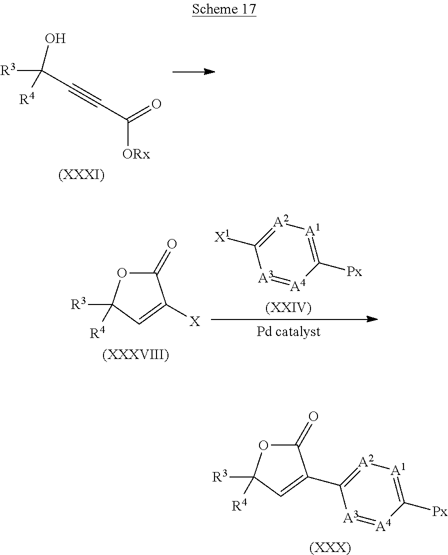

35) Compounds of formula (XXXVIII) where X is boron derivative can be prepared from compounds of formula (XXXI) (see scheme 14) using the same procedure as is described in scheme 2) using a borane derivative, such as pinacol borane or catecholborane in a suitable solvent such as tetrahydrofuran or dichloromethane, in the presence of a copper hydride, e.g. as is described in Angewandte Chemie, International Edition (2008), 47(52), 10183-10186. Alternatively, compounds of formula (XXXVIII) when X is a boron derivative, can be prepared from compounds of formula (XXXI) using the same procedure as is described in scheme 2) using a diboron derivative, such as pinacol diborane, in the presence of a suitable catalyst, such as copper, palladium of nickel, as is described for example in Angewandte Chemie, International Edition (2009), 48(12), 2192-2195.
36) Compounds of formula (XXX) can be prepared from compounds of formula (XXIV) and (XXXVIII) when X is a boron derivative and X¹ is a leaving group for example a halogen, such as bromo, or C(O)R wherein R is halogen, OH or C₁-C₁₅alkoxy, using a Suzuki reaction, e.g. as described in 22).
37) Compounds of formula (XXXVIII) where X is a tin derivative can be prepared from compounds of formula (XXXI) (see scheme 14) using the same procedure as is described in 2), e.g. using a trialkyltin hydride compound of formula (IX) in the presence of a palladium catalyst, or a copper catalyst, as is described in Organic Letters (2005), 7(23), 5249-5252.
38) Compounds of formula (XXX) can be prepared from compounds of formula (XXIV) and (XXXVIII) when X is a tin derivative and X¹ is a leaving group, for example a halogen, such as bromo, using the methods described in 7).

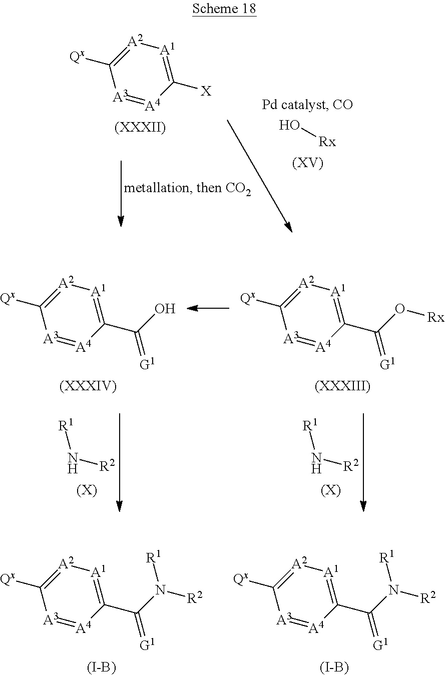

39) Compounds of formula (I-B) can be made by treatment of a compound of formula (XXXIV) with a compound of formula (X) using the same methods as described in 11).
40) Alternatively, compounds of formula (I-B) can be made from a compound of formula (XXXIII) wherein Rx is C₁-C₆alkoxy by heating the ester and an amine of formula (X) together in a thermal process Amines of formula (X) are known in the literature or can be prepared using methods known to a person skilled in the art.
41) Compounds of formula (XXXIV), can be made by treatment of a compound of formula (XXXII), wherein X is a halogen, for instance bromine, using the same methods as described in 13).
42) Compounds of formula (XXXIV) can be made by hydrolysis of a compound of formula (XXXIII), and Rx is C₁-C₆alkyl, such as methyl or tert-butyl using the same methods as described in 14).
43) Compounds of formula (XXXIII) wherein Rx is C₁-C₆alkoxy, can be prepared by reacting a compound of formula (XXXII) wherein X is a leaving group, for example a halogen, such as bromo, with an alcohol of formula Rx-OH, using the same methods as described in 15).

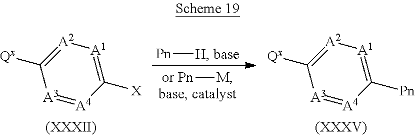

44) Compounds of formula (XXXV), wherein Pn is an optionally substituted heterocycle, can be made, for example in the case where the heterocycle is attached via a nitrogen atom, by treatment of a compound (XXXII) wherein X is a halogen, such as fluorine, with a heterocyclic compound Pn-H and a suitable base, such as potassium carbonate. Alternatively, compounds of formula (XXXV), wherein Pn is an optionally substituted heterocycle, can be made, for example in the case where the heterocycle is attached via a carbon atom, by treatment of a compound (XXXII) wherein X is a halogen, such as bromine, with a heterocyclic compound Pn-M, wherein M is hydrogen or a metal, such as boron, magnesium or zinc, in which case M can be optionally substituted, with a base and a suitable catalyst, such as a palladium or a copper catalyst, in the presence of a suitable ligand for the catalyst, such as, for example, a diamine ligand, or a phosphine ligand. Such reactions are carried out at a temperature of from −120° C. to +130° C., preferably from −100° C. to 100° C.
45) The compounds of formula (IA) can be obtained from racemic mixtures by chiral separation using HPLC.

In the above descriptions reference to leaving groups includes leaving groups such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. the leaving group may be selected from —N₂⁺Cl⁻, —N₂⁺BF₄⁻, —N₂⁺Br⁻, —N₂⁺PF₆⁻) and phosphonate esters (e.g. —OP(O)(OR)₂, wherein R is methyl or ethyl).

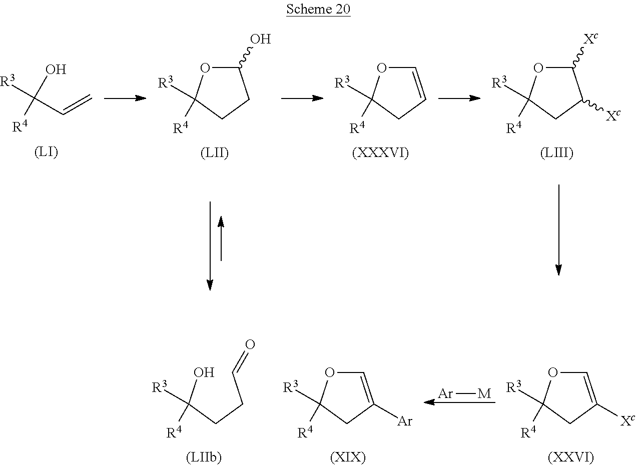

In scheme 20 Ar stands for group A

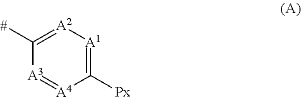

wherein A¹, A², A³, A⁴ are as defined for compounds of formula (IA) and Px is as defined for compounds of formula (XIX). In scheme 20× stands for a halogen (X═Cl, Br, I); M stands for a derivative of B, Si, Sn, Mg, Zn, Mn.
46) Compounds of formula LII can be obtained from compounds of formula LI via hydroformylation, e.g. by reacting compounds of formula LI with CO and H₂ in the presence of a suitable catalyst. Structure LII comprises any composition of cyclic stereo-isomers and or of open chain structure LIIb isomers.

Suitable catalysts for the hydroformylation reaction are complexes of transition metals (rhodium, cobalt, platinum, palladium, iridium) preferably rhodium, preferably with a suitable ligand. Particularly preferred ligands include hydride, carbonyl, halogen, substituted and unsubstituted cyclopentadienyls, 2,4-alkanedionates (e.g. acetyacetonate), phosphorus derivatives and mixtures thereof. Phosphorus derivatives are preferred and are typically represented by the formula P(R)₃ wherein R is an aryl, alkyl, alkoxy, aryloxy, alkylamino, arylamino or a bidentate ligand of the formula (R)₂P—Y—P(R)₂, Y represents a 1-20 atom linker. Each R groups may be the same or different.

Preferred ligands are monodentate and bidentate phospines, phosphites, phosphinites. Preferred specific ligands are e.g. triphenyl phosphine, triphenyl phosphite, BIPHEPHOS (2,2′-Bis[(1,1′-biphenyl-2,2′-diyl)phosphite]-3,3′-di-tert-butyl-5,5′-dimethoxy-1,1′-biphenyl), 6-DPPon (6-(diphenylphosphino)-2(1H)-pyridinone), BISBI (2,2′-Bis[(diphenylphosphino)methyl]-1,1′-biphenyl), NAPHOS (2,2′-Bis(diphenylphosphinomethyl)-1,1′-binaphthalene), XANTPHOS (9,9-Dimethyl-4,5-bis(diphenylphosphino)xanthene), tBu-XANTPHOS (1,1′-[2,7-bis(1,1-dimethylethyl)-9,9-dimethyl-9H-xanthene-4,5-diyl]bis[1,1-diphenylphosphine]), TPPTS (3,3′,3″-phosphinidynetris[benzenesulfonic acid] trisodium salt), Tris(2,4-bis(1,1-dimethylethyl)phenyl)-phosphite.

The catalyst may be formed in situ from a catalyst precursor (such as acetylacetonato)dicarbonyl rhodium, tris(triphenylphosphine)rhodium carbonyl hydride, Rh₆(CO)₁₆, Rh₂O₃, RhCl₃, [Rh(OMe)COD]₂, [Rh₂(OAc)₄], [RhCl(COD)]₂) and a suitable ligands or preformed in a separate step. A preferred catalyst precursor to ligand ratio is between 1:1 to 1:100 more preferably between 1:5 to 1:50.

The reaction temperature is preferably in the range of 0-250° C. more preferably at 50-150° C. The reaction pressure is preferably in the range of 1-200 bar more preferably 10-100 bar (an atmosphere of carbon monoxide and hydrogen). The reaction time is usually in the range 1 h to 100 h.

The molar ration of CO:H₂ is preferably 1:100 to 100:1 more preferably 1:5 to 5:1. Optionally, CO and/or H₂ reactants may be generated in situ from formaldehyde, formic acid derivates, metal carbonyls or other suitable precursors.

Preferred solvents include C₅-C₂₀ aliphatic hydrocarbons, C₆-C₂₀ aromatic hydrocarbons, halogenated hydrocarbons, alcohols, ethers, esters, amides, and mixtures thereof
47) Compounds of formula XXXVI may be prepared from compounds of formula LII by dehydration (elimination of water) in the presence of a suitable acidic catalyst or a suitable activation agent (carboxylic or sulfonic acid chloride or anhydride) and a suitable base (Et₃N, pyridine, DBU). The acid catalyst is preferably p-toluenesulfonic acid, methane sulfonic acid or pyridinium p-toluenesulfonate. Relative amount of the catalyst to substrate is preferably 1-100 mol %. The reaction may be further facilitated by the presence of a drying agent (Na₂SO₄, molecular sieves), azeotropic distillation, gas flow through the reaction mixture, application of vacuum (vacuum distillation, flash vacuum pyrolysis) or other means of removing the water formed. Reaction temperature is in the range 0° C. to 1000° C., more preferably 50° C. to 200° C. Reaction pressure is preferably between 0.1 mbar and atmospheric, most between 0.1 to 200 mbar. The reaction time is usually in the range 0.1 h to 100 h. The product of the hydroformylation reaction LII may be isolated and or purified before the dehydration or alternatively the conversion to XXXVI may be carried in the same pot as the hydroformylation reaction (one pot reaction).
48) Compounds of formula LIII may be prepared from compounds of formula VXXXVI by reacting compounds of formula VI with a halogen X (X represents Cl, Br, I). Structure LIII comprises any composition of cyclic stereo-isomers. Suitable solvents include polar and non-polar organic solvents e.g. dichloromethane, chloroform, dichloroethane, dioxane, ethyl acetate, acetonitrile, THF. The reaction temperature is usually in the range −78° C. to 100° C., more preferably −78° C. to 0° C. The reaction time is usually in the range 0.1 h to 100 h.
49) Compounds of formula XXVI, may be prepared from compounds of formula LIII by elimination of HX^{C}, preferably in the presence of a suitable base and solvent. Suitable bases include Et₃N, diisopropyl ethyl amine, pyridine, DBU, DBM, iPrMgCl, iPrMgBr, LDA. Suitable solvents include polar and non-polar organic solvents e.g. dichloroethane, dioxane, THF, toluene, DMF, NMP, acetonitrile. The reaction temperature is usually in the range −30° C. to 200° C., more preferably 0° C. to 150° C. The reaction time is usually in the range 0.1 h to 100 h.
50) Compounds of formula XIX can be obtained from compounds of formula LIII via a coupling reaction (e.g. Suzuki, Stille, Hiyama, Kumada, Negishi) e.g. by treating compounds of formula VII, with a reactant Ar-M, wherein Ar are as defined above and M represents a suitable derivative of B, Si, Sn, Mg, Zn, Mn (e.g. boronic acid, boronic ester, trifluoroborate, dialkyl-hydroxysilane, trialkyltin, MgCl, MgBr, ZnCl, ZnBr, MnCl) in presence of a catalyst and optionally in the presence of a suitable ligand, solvent and additive. Suitable catalysts are e.g. palladium catalysts such as Pd(OAc)₂, PdCl₂, Pd₂(dba)₃, Pd₂(dba)₃.CHCl₃, [Pd(PPh₃)₄], [Pd(Cl)₂(H₃CCN)₂)], [(allyl)Pd(Cl)]₂, [Pd(PPh₃)₂(Cl)_{2]}, [Pd(DPPF)(Cl)₂], PEPPSI, nickel catalysts such as NiCl₂, Ni(OAc)₂, Ni(acac)₂, [Ni(PPh₃)₂Cl₂], [Ni(DPPP)Cl₂]. Suitable ligands are e.g. phosphine ligands such as P(tBu)₃, tris(ortho-tolyl)phosphine, B1NAP, PPh₃, PCy₃, S-Phos, X-Phos, Ru-Phos, trifuryl phosphine, Tris(2,4-bis(1,1-dimethylethyl)phenyl)-phosphite, DPEphos, Josiphos and carbine ligands such as IMes, SIMes, IPr, SIPr. Suitable solvents include polar and non-polar organic solvents e.g. DMF, DMA, DME, dioxane, NMP, toluene, xylene, water, AcCN, THF, ionic liquids. Suitable additives are e.g. trialkyl amine, metal carbonate or acetate or phosphate or fluoride. Examples of additives are e.g. Et₃N, Na₂CO₃, K₂CO₃, Cs₂CO₃, K₃PO₄, KF, CsF. The reaction temperature is usually in the range 0° C. to 200° C., more preferably 50° C. to 150° C. The reaction time is usually in the range 1 h to 100 h.

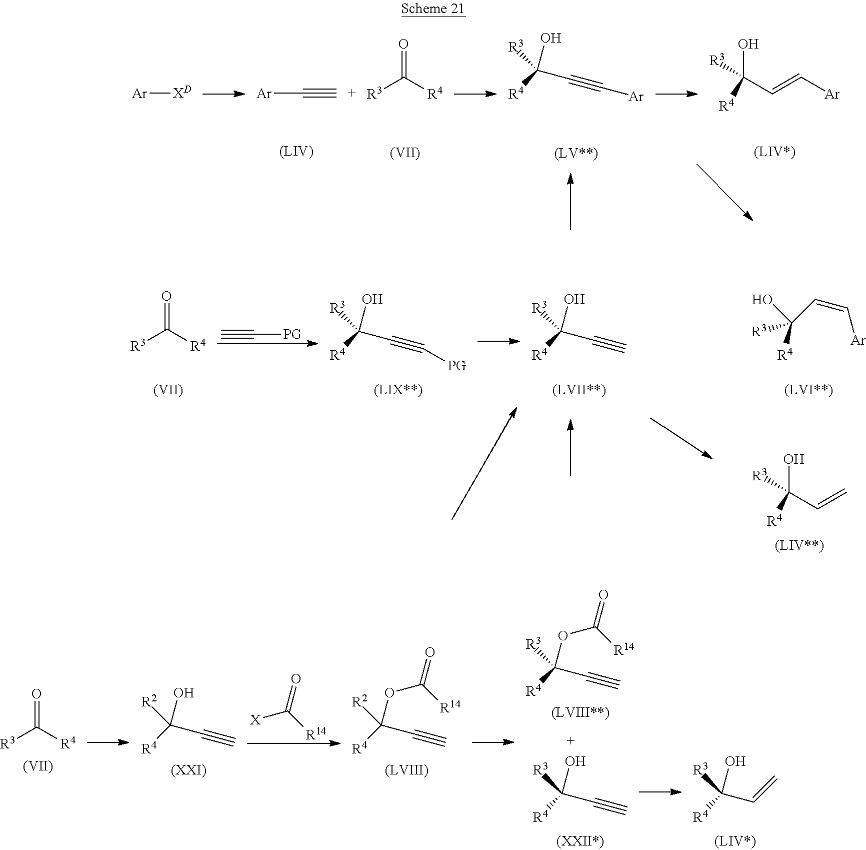

In scheme 21 Ar stands for group A

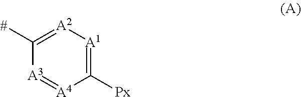

wherein A¹, A², A³, A⁴ are as defined for compounds of formula (IA) and Px is as defined for compounds of formula (XIX). In scheme 21 X^{D} represents a halogen (Cl, Br, I) or a pseudohalogen (OTf, OTs, diazonium).
51) Compounds of formula LIV can be obtained from the corresponding aryl halide via a coupling reaction (e.g. Suzuki, Stille, Hiyama, Kumada, Negishi, Sonigashira) e.g. by treating the Ar—X^{D}, wherein Ar are as defined above and X^{D} represents a halogen (Cl, Br, I) or a pseudohalogen (OTf, OTs, diazonium) with an ethynyl-M, wherein Ar are as defined above and M represents a suitable derivative of B, Si, Sn, Mg, Zn, Cu (formed in situ from corresponding terminal alkyne) in presence of a catalyst and optionally in the presence of a suitable ligand, solvent and additive. Suitable catalysts are e.g. palladium catalysts such as Pd(OAc)₂, PdCl₂, Pd₂(dba)₃, Pd₂(dba)₃.CHCl₃, [Pd(PPh₃)₄], [Pd(Cl)₂(H₃CCN)₂)], [(allyl)Pd(Cl)]₂, [Pd(PPh₃)₂(Cl)₂], [Pd(DPPF)(Cl)₂], PEPPSI, Suitable solvents include polar and non-polar organic solvents e.g. DMF, DMA, DME, dioxane, NMP, toluene, xylene, water, AcCN, THF, ionic liquids. Suitable additives are e.g. trialkyl amine, metal carbonate or acetate or phosphate or fluoride. Examples of additives are e.g. Et₃N, Na₂CO₃, K₂CO₃, Cs₂CO₃, K₃PO₄, KF, CsF. The reaction temperature is usually in the range 0° C. to 200° C., more preferably 50° C. to 150° C. The reaction time is usually in the range 1 h to 100 h.
52) Enantiomerically enriched compounds of formula LV** wherein R³ and R⁴ are as defined for compounds of formula I and wherein Ar are as defined above can be prepared by deprotonating compounds of formula LIV using a suitable base in a suitable aprotic organic solvent between −90° C. and 80° C., followed by reaction with a titanium alkoxide or chloroalkoxide e.g. Ti(OiPr)₄, Ti(OEt)₄, ClTi(OiPr)₃ between −40° C. and 60° C. in the presence of chiral amino alcohols ligands or chiral diol ligands, a suitable additive and compounds of formula VII, as described in Angewandte Chemie, International Edition (2011), 50(15), 3538-3542. Suitable base are BuLi, sec-BuLi, tert-BuLi, Me₂Zn, Et₂Zn, Me₃Al, Et₃Al. Suitable solvents are xylenes, toluene, THF, DME, CH₂Cl₂, C₂H₄Cl₂. The preferred solvent is toluene. Suitable chiral ligands are Cinchona alkaloids (e.g. quinine, quinidine, cinchonidine, cinchonine), N,N-dialkylephedrine, N,N-dialkylpseudoephedrine, (R)-binol, (R)—H₈-binol. Preferred ligands are Cinchona alkaloids. Suitable additives are CaH₂ and BaF₂. The preferred reaction temperature is between −30° C. and 30° C.
53) Enantiomerically enriched compounds of formula LV** wherein R³ and R⁴ are as defined for compounds of formula I and wherein Ar are as defined above can be prepared by deprotonating compounds of formula LIV using a suitable organolithium base e.g. BuLi between −100 and −40° C. in an aprotic organic solvent (e.g. toluene, tetrahydrofuran, 1,2-dimethoxyethane, 1,4-dioxane, diethylether, CH₂Cl₂, C₂H₄Cl₂) in presence of a chiral diol ligand (e.g. (S)-1-(2-hydroxy-3-phenyl-1-naphthyl)-3-phenyl-naphthalen-2-ol) and compounds of formula VII as described in Chem. Commun. 2011, 47, 5614.
54) Enantiomerically enriched compounds of formula LIX** wherein R³ and R⁴ are as defined for compounds of formula I and wherein Ar are as defined above can be obtained by reduction of compounds of LV** by sodium bis(2-methoxyethoxy)aluminumhydride as described in Tetrahedron, 66(39), 7726-7731; 2010. Suitable solvents for this reaction are toluene, tetrahydrofuran, 1,2-dimethoxyethane, 1,4-dioxane and diethylether. The reaction is run between −78° C. and 25° C. and preferably between −50° C. and −10° C.
55) Enantiomerically enriched compounds of formula LVI** wherein R³ and R⁴ are as defined for compounds of formula I and wherein Ar are as defined above can be obtained by reduction of compounds of LV** by deactivated palladium catalysts (e.g. Lindlar's catalyst) as described in Tetrahedron, 53(11), 3879-3916; 1997. The suitable solvents for this reaction are ethyl acetate, tetrahydrofuran, 1,2-dimethoxyethane, 1,4-dioxane, diethylether, methanol and ethanol. The reaction is run between −0° C. and 100° C. and preferably between 10 and 60° C.
56) Compounds of formula XXII wherein R³ and R⁴ are as defined for compounds of formula I can be prepared by addition of ethynylnucleophiles (e.g. ethynylmagnesium bromide, ethynylmagnesium chloride) to the ketone of formula VII, e.g. using similar conditions as described in Advanced Synthesis & Catalysis, 349(8+9), 1393-1398; 2007.
57) Compounds of formula LVIII wherein R³ and R⁴ are as defined for compounds of formula I and R¹⁴ is C₁-C₁₂alkyl, preferably C₁-C₈ alkyl, are prepared by reacting compounds of formula XXII using similar conditions as described in Advanced Synthesis & Catalysis, 349(8+9), 1393-1398; 2007.
58) Enantiomerically enriched compounds of formula XXII* and LVIII** wherein R³ and R⁴ are as defined for compounds of formula I and R¹⁴ is C₁-C₁₂alkyl, preferably C₁-C₈ alkyl are prepared by treating compounds of formula LVIII with a suitable hydrolase enzyme in a suitable aqueous system in presence of a suitable buffer, pH 5-9, between 10° C. and 80° C. Suitable enzymes are Pig liver esterase (Roche), Novozyme 398 (Novozymes), Novozymes 435 (supported lipase, Novozymes), Alcalase from Bacillus licheniformis (Merck), Alcalase (Novozymes), Protease type XIII from Aspergillus oryzae (Sigma), Lipase from Candida rugosa (Sigma), Lipase type VII from Candida rugosa (Sigma), Palatase, lipase from Rhizomucor miehei (Sigma), Wheat germ lipase (Sigma), Lipase PS from Burkholderia cepacia (Amano), Lipase AK from Pseudomonas fluorescens (Amano), Lipase from porcine pancreas (Sigma), Esterase ECS-Es 01 (Enzymicals), Esterase ECS-Es 06 (Enzymicals), Esterase ECS-Es 08 (Enzymicals), Esterase ECS-Es 09 (Enzymicals), Esterase ECS-Es 10 (Enzymicals), Lipase MY from Candida rugosa (Meito Sangyo), Lipase OF from Candida rugosa (Meito Sangyo), Lipase SL from Burkholderia cepacia (Meito Sangyo), Lipase TL from Pseudomonas stutzeri (Meito Sangyo). The preferred enzymes are Lipase from Candida rugosa (Sigma), Lipase type VII from Candida rugosa (Sigma), Lipase MY from Candida rugosa (Meito Sangyo), Lipase OF from Candida rugosa (Meito Sangyo). The suitable solvents systems are water, water/dimethylsulfoxide, water/toluene, water/acetone, water/methanol, water/acetonitrile, water/1,4-dioxane, water/n-hexane, water/cyclohexane, water/methyl-tert-butylether, water/diisopropylether. The preferred solvent systems are water/dimethylsulfoxide, water/methanol, water/acetone, water/n-hexane, water/cyclohexane. The preferred buffers are NaH₂PO₄/Na₂HPO₄ and KH₂PO₄/K₂HPO₄. The preferred pH is 7.4. The preferred temperature is between 35 and 55° C.
59) Enantiomerically enriched compounds of formula LVII** a wherein R³ and R⁴ are as defined for compounds of formula I and R¹⁴ is C₁-C₁₂alkyl, preferably C₁-C₈ alkyl are prepared by treating compounds of formula LVIII with a suitable hydrolase enzyme in a suitable aqueous system in presence of a suitable buffer, pH 5-9, between 10° C. and 80° C. Preferred enzymes are Lipase QLM from Alcaligenes sp. (Meito Sangyo) and Lipase PL from Alcaligenes sp. (Meito Sangyo). The preferred solvent systems are water/dimethylsulfoxide, water/methanol, water/acetone, water/n-hexane, water/cyclohexane. The preferred buffers are NaH₂PO₄/Na₂HPO₄ and KH₂PO₄/K₂HPO₄. The preferred pH is 7.4. The preferred temperature is between 35 and 55° C.
60) Enantiomerically enriched compounds of formula LIX** wherein R³ and R⁴ are as defined for compounds of formula I and wherein PG is an organosilicon, preferably trialkylsilyl and most preferably trimethylsilyl, are prepared by deprotonation of ethynyl-PG with a suitable organolithium (e.g. BuLi) in presence of a suitable chiral modifier, preferably aminoalcohols ligand in a aprotic organic solvent between −80° C. and 25° C. The preferred chiral ligand are dialkylephedrine and dialkylpseudoephedrine. The most preferred chiral ligand is (1R,2S)-1-phenyl-2-pyrrolidin-1-yl-propan-1-ol. The preferred solvent is tetrahydrofuran. The preferred temperature is between −70° C. and 20° C.
61) Enantiomerically enriched compounds of formula LVII** wherein R³ and R⁴ are as defined for compounds of formula I are prepared by reactions of compounds of formula LIX** wherein PG is an organosilicon, preferably trialkylsilyl and most preferably trimethylsilyl, with a suitable base or fluorine source in a suitable organic solvent. Preferred bases are tetrabutylammonium fluoride, potassium carbonate and sodium carbonate. Preferred solvent are tetrahydrofuran, ethanol and methanol. Suitable reaction temperatures is between −10° C. and 60° C.
62) Enantiomerically enriched compounds of formula LIV** wherein R³ and R⁴ are as defined for compounds of formula I can be obtained by reduction of compounds of LVII** by deactivated palladium catalysts (e.g. Lindlar's catalyst) as described in Tetrahedron, 53(11), 3879-3916; 1997. The suitable solvents for this reaction are ethyl acetate, tetrahydrofuran, 1,2-dimethoxyethane, 1,4-dioxane, diethylether, methanol and ethanol. The reaction is run between −0° C. and 100° C. and preferably between 10 and 60° C.
63) Enantiomerically enriched compounds of formula LIV* wherein R³ and R⁴ are as defined for compounds of formula I can be obtained by reduction of compounds of XXII* by deactivated palladium catalysts (e.g. Lindlar's catalyst) as described in Tetrahedron, 53(11), 3879-3916; 1997. The suitable solvents for this reaction are ethyl acetate, tetrahydrofuran, 1,2-dimethoxyethane, 1,4-dioxane, diethylether, methanol and ethanol. The reaction is run between −0° C. and 100° C. and preferably between 10 and 60° C.

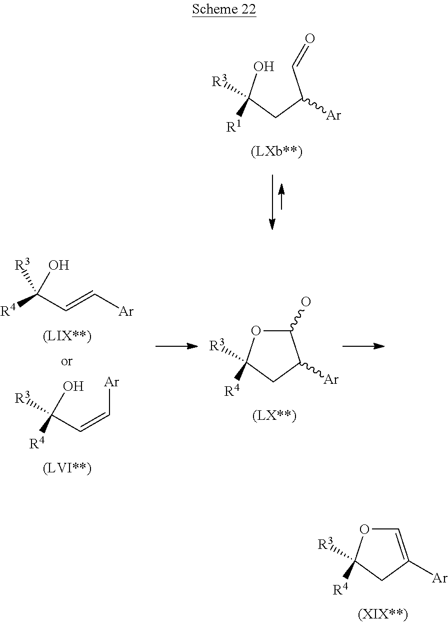

In scheme 22 Ar stands for group A

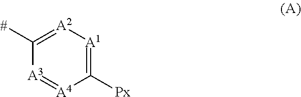

wherein A¹, A², A³, A⁴ are as defined in the claims and Px is as defined for compounds of formula (XIX).
64) Compounds of formula LX**, wherein Ar is as defined above and wherein R³ and R⁴ are as defined for compounds of formula (IA), may be prepared via hydroformylation of compounds of formula LIX** or LVI**, e.g. by reacting compounds of formula LIX** with CO and H₂ in the presence of a suitable catalyst. Structure LX** comprises any composition of cyclic stereo-isomers and or of open chain structure LXb** isomers.

Suitable catalysts for the hydroformylation reaction are complexes of transition metals (rhodium, cobalt, platinum, palladium, iridium) preferably rhodium, preferably with a suitable ligand. Particularly preferred ligands include hydride, carbonyl, halogen, substituted and unsubstituted cyclopentadienyls, 2,4-alkanedionates (e.g. acetylacetonate), phosphorus derivatives and mixtures thereof. Phosphorus derivatives are preferred and are typically represented by the formula P(R)₃ wherein R is an aryl, alkyl, alkoxy, aryloxy, alkylamino, arylamino or a bidentate ligand of the formula (R)₂P—Y—P(R)₂, Y represents a 1-20 atom linker. Each R groups may be the same or different.

Preferred ligands are bulky, π-acceptor phosphines, phosphites, phosphinite, phosphabenzenes, phosphabarrelenes, PArₓR₃₋ₓ (x=0-2; R=pyrrolyl, indolyl, carbazolyl; Ar=aryl, e.g. phenyl), preferably phosphites, phosphabenzenes, phosphinolines and phosphaadamantanes. Preferred specific ligands are e.g. Triphenyl phosphite, BIPHEPHOS, tris(hexafluoroisopropyl)phosphite, Tris(2,4-bis(1,1-dimethylethyl)phenyl)-phosphite, Tris(2-(1,1-dimethylethyl)phenyl)-phosphite, Tris(2-(1,1-dimethylethyl)-4-methyl-phenyl)-phosphite, 2,4,6-Triphenylphosphabenzene, 2,3,4,5,6-pentaphenylphosphabenzene, 2,3,5,6-tetraphenylphosphabenzene, 2,6-bis(2,4-dimethylphenyl)-4-phenylphosphabenzene, 2,6-bis(2-methylphenyl)-4-phenylphosphabenzene, 4-phenyl-2,6-bis(2,4,5-trimethylphenyl)phosphabenzene, 2,6-di-2-naphthalenyl-4-phenylphosphabenzene, 2-(2-naphthalenyl)-4,6-diphenylphosphabenzene, 2,6-bis(1-methylethyl)-4-phenylphosphabenzene, 2,4,6-tris(1,1-dimethylethyl)phosphabenzene, 2,6-dimethyl-4-phenylphosphabenzene, 2,4,10-triphenyl-4H-1,4-ethenophosphinoline, 2,10-bis(1-methylethyl)-4-phenyl-4H-1,4-ethenophosphinoline, 2,10-bis(2,4-dimethylphenyl)-4-phenyl-4H-1,4-ethenophosphinoline, 2,10-bis(2,4-dimethylphenyl)-6-methyl-4-phenyl-4H-1,4-ethenophosphinoline, 2,10-bis(2,4-dimethylphenyl)-7-methyl-4-phenyl-4H-1,4-ethenophosphinoline, 1,3,5,7-tetramethyl-6-phenyl-2,4,8-trioxa-6-phosphaadamantane, 1,3,5,7-tetraethyl-6-phenyl-2,4,8-trioxa-6-phosphaadamantane.

The catalyst may be formed in situ from a catalyst precursor (such as (acetylacetonato)dicarbonyl rhodium, tris(triphenylphosphine)rhodium carbonyl hydride, Rh₆(CO)₁₆, Rh₂O₃, RhCl₃, [Rh(OMe)COD]₂, [Rh₂(OAc)₄], [RhCl(COD)]₂) and a suitable ligands or preformed in a separate step. A preferred catalyst precursor to ligand ratio is between 1:1 to 1:100 more preferably between 1:5 to 1:50.

The reaction temperature is preferably in the range of 0-250° C. more preferably at 50-150° C. The reaction pressure is preferably in the range of 1-200 bar more preferably 10-100 bar (an atmosphere of carbon monoxide and hydrogen). The reaction time is usually in the range 1 h to 100 h.

The molar ration of CO:H₂ is preferably 1:100 to 100:1 more preferably 1:5 to 5:1. Optionally, CO and/or H₂ reactants may be generated in situ from formaldehyde, formic acid derivatives, metal carbonyls or other suitable precursors.

Preferred solvents include C₅-C₂₀ aliphatic hydrocarbons, C₆-C₂₀ aromatic hydrocarbons, halogenated hydrocarbons, alcohols, ethers, esters, amides, and mixtures thereof. For liquid substrates the reaction may be performed neat.

65) Compounds of formula (XIX), wherein Ar is as defined above and wherein R³ and R⁴ are as defined for compounds of formula (IA), may be prepared from compounds of formula LX** by dehydration (elimination of water) in the presence of a suitable acidic catalyst or a suitable activation agent (carboxylic or sulfonic acid chloride or anhydride) and a suitable base (Et₃N, pyridine, DBU). The acid catalyst is preferably p-toluenesulfonic acid or pyridinium p-toluenesulfonate. Relative amount of the catalyst to substrate is preferably 1-100 mol % more preferably 1:10-30 mol %. The reaction may be further facilitated by the presence of a drying agent (Na₂SO₄, molecular sieves), azeotropic distillation, gas flow through the reaction mixture, application of vacuum or other means of removing the water formed. Reaction temperature is in the range 0° C. to 200° C., more preferably 50° C. to 150° C. Reaction pressure is preferably between 0.1 mbar and atmospheric, most preferably atmospheric. The reaction time is usually in the range 1 h to 100 h. The product of the hydroformylation reaction LX** may be isolated and or purified before the dehydratation or alternatively the conversion to (I) may be carried in the same pot as the hydroformylation reaction (one pot reaction).

Hydroformation reactions, including reaction conditions and suitable catalyst, are described in Breit et al., Chem. Comm., 2004, 694-695, Fuchs et al., Chem. Eur. J., 2006, 12, 6930-6939, and Breit et al., Chem. Eur. J., 2001, 7, No. 14, each of which is incorporated by reference.

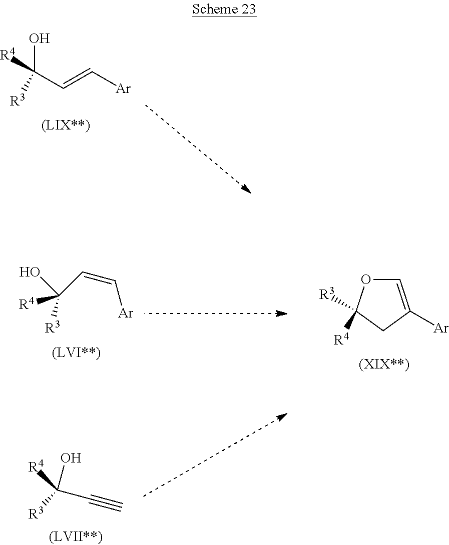

66) Compounds of formula XIX** may be prepared from compounds of formula LIX**, LVI** and LVII** following the procedures set out above.

The reaction described above are also applicable to synthesis of racemic mixtures.

In a further aspect the invention provides a process for preparing a compound of formula (XIX)

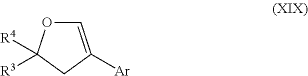

wherein
Ar is group A

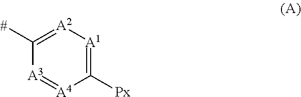

R³, R⁴, A¹, A², A³, A⁴ are as defined for the compound of formula (IA), and Px is P as defined for the compound of formula (IA), a leaving group X^{B}, such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. X^{B} is —N₂⁺Cl⁻, —N₂⁺BF₄⁻, —N₂⁺Br⁻, —N₂⁺PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride, or C(O)R wherein R is halogen, OH or C₁-C₁₅alkoxy, or a salt or N-oxide thereof;
comprising dehydrating a compound of formula (LX)

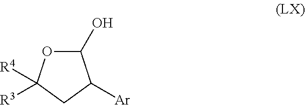

wherein R³, R⁴ and Ar are as defined for the compound of formula (XIX);
with a suitable acidic catalyst or a suitable activation agent and a suitable base. Examples of preferred reaction conditions are described in paragraph 65.

The compound of formula (LX) may be prepared by reacting a compound of formula (LIX) or (LVI)

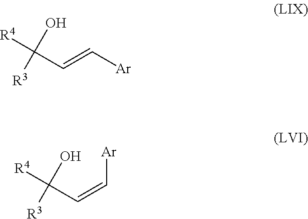

wherein R³, R⁴ and Ar are as defined for the compound of formula (XIX);
with a source of H₂ and CO in the presence of a catalyst comprising a complex of a transition metal and a suitable ligand. Examples of preferred reaction conditions are described in paragraph 64.

In a further aspect the invention provides a process for the preparation of a compound of formula (LX) comprising reacting a compound of formula (LIX) with a source of H₂ and CO in the presence of a catalyst comprising a complex of a transition metal and a suitable ligand. Examples of preferred reaction conditions are described in paragraph 64.

In particular, the complex of a transition metal is preferably a rhodium complex and the ligand is preferably a phosphite, phosphabenzene, phosphinoline or phosphaadamantane ligand. The source of hydrogen and CO reactants may be gaseous CO and/or H₂ or generated in situ e.g. from formaldehyde, formic acid derivates, metal carbonyls or other suitable precursors.

In a further aspect the invention provides a process for the preparation of a compound of formula (XXXVI)

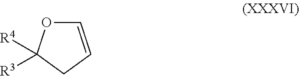

wherein R³ and R⁴ are as defined for the compound of formula (IA) comprising dehydrating a compound of formula (LII)

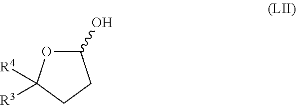

with a suitable acidic catalyst or a suitable activation agent and a suitable base. Examples of preferred reaction conditions are described in paragraph 47.

The compound of formula (LII) may be prepared by reacting a compound of formula (LI)

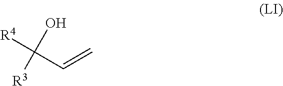

with a source of H₂ and CO in the presence of a catalyst comprising a complex of a transition metal and a suitable ligand. Examples of preferred reaction conditions are described in paragraph 46.

The reaction may include the additional step of reacting the compound of formula (XXXVI) with chlorine, bromine or iodine to give a compound of formula (LIII)

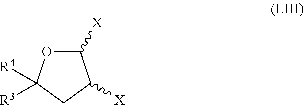

wherein R³ and R⁴ are as defined for the compound of formula (XIX) and each X is independently Cl, Br or I. Examples of preferred reaction conditions are described in paragraph 48.

The reaction may include the additional step of eliminating HX from the compound o formula (LIII) e.g. in the presence of a suitable base, to give a compound of compound of formula (XXVI)

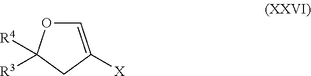

wherein R³ and R⁴ are as defined for the compound of formula (IA) and X is Cl, Br or I. Examples of preferred reaction conditions are described in paragraph 49.

The reaction may also include the additional step of reacting a compound of formula (XXVI) with a compound of formula Ar-M, wherein Ar is group A

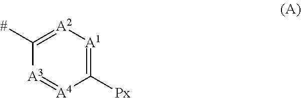

wherein A¹, A², A³, A⁴ are as defined for the compound of formula (IA), and Px is P as defined for the compound of formula (IA), a leaving group, such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. X^{B} is —N₂⁺Cl⁻, —N₂⁺BF₄⁻, —N₂⁺Br⁻, —N₂⁺PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride, or C(O)R wherein R is halogen, OH or C₁-C₁₅alkoxy; to give a compound of formula

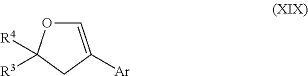

wherein R³ and R⁴ are as defined for the compound of formula (IA) and Ar is as defined above. Examples of preferred reaction conditions are described in paragraph 50.

In a further aspect the invention provides a process for the preparation of a compound of formula (LII) comprising reacting a compound of formula (LI) with a source of H₂ and CO in the presence of a catalyst comprising a complex of a transition metal and a suitable ligand. Examples of preferred reaction conditions are described in paragraph 46.

The complex of a transition metal is preferably a rhodium complex and the ligand is preferably a phosphite, phosphabenzene, phosphinoline or phosphaadamantane ligand. The source of hydrogen and CO reactants may be gaseous CO and/or H₂ or generated in situ e.g. from formaldehyde, formic acid derivates, metal carbonyls or other suitable precursors.

Intermediates useful in this process are also form additional aspects of the invention.

Accordingly, a group of novel intermediates are compounds of formula (LX)

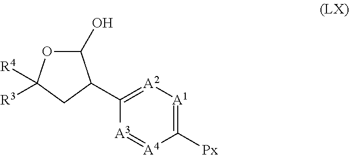

wherein R³, R⁴, A¹, A², A³, A⁴ are as defined for the compound of formula (IA) and Px is P as defined for the compound of formula (IA), a leaving group X^{B}, such as halogen, C₁-C₈alkoxy, C₁-C₈alkylsulfonyloxy, C₁-C₈haloalkylsulfonyloxy, C₁-C₈arylsulfonyloxy, optionally substituted C₁-C₈arylsulfonyloxy (aryl is preferably phenyl), diazonium salts (e.g. X^{B} is —N₂⁺Cl⁻, —N₂⁺BF₄⁻, —N₂⁺Br⁻, —N₂+PF₆⁻), phosphonate esters (e.g. —OP(O)(OR)₂, wherein R is methyl or ethyl), preferably bromo, iodo, chloro, trifluoromethylsulfoxy, p-toluenesulfoxy, diazonium chloride, or C(O)R wherein R is halogen, OH or C₁-C₁₅alkoxy, or a salt or N-oxide thereof. The preferences for R³, R⁴, A¹, A², A³, A⁴ and Px (when Px is P) are the same as the preferences set out for the corresponding substituents of the compound of formula (IA).

One group of compounds of formula (LX) are compounds of (LXa)

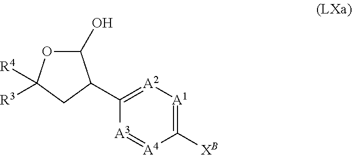

wherein R³, R⁴, A¹, A², A³, A⁴ are as defined for the compound of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of the compound of formula (IA).

Another group of compounds of formula (LX) are compounds of (LXb)

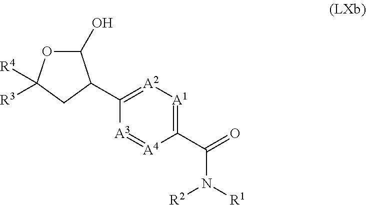

wherein R¹, R², R³, R⁴, A¹, A², A³ and A⁴ are as defined for the compound of formula (IA), or a salt or N-oxide thereof. The preferences for R¹, R², R³, R⁴, A¹, A², A³, A⁴ are the same as the preferences set our for the corresponding substituents of the compound of formula (IA).

Another group of compounds of formula (LX) are compounds of (LXc)

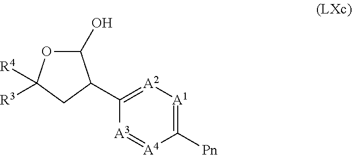

wherein A¹, A², A³, A⁴, R³ and R⁴ are as defined for the compounds of formula (IA), and Pn is a heterocyclyl or heteocyclyl substituted by one to five Z, as defined for compounds of formula I. The preferences for A¹, A², A³, A⁴, R³, R⁴ and heterocyclyl are the same as the preferences set out for the corresponding substituents of a compound of formula (IA).

Another group of novel intermediates are compounds of formula (LIX)

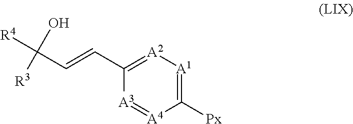

wherein R³, R⁴, A¹, A², A³, A⁴ are as defined for the compound of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of the compound of formula (IA).

One group of compounds of formula (LIX) are compounds of (LIXa)

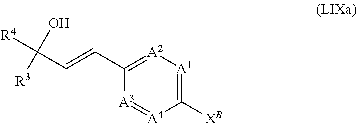

wherein R³, R⁴, A¹, A², A³, A⁴ are as defined for the compound of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of the compound of formula (IA).

Another group of compounds of formula (LIX) are compounds of (LIXb)

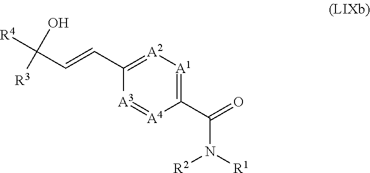

wherein R¹, R², R³, R⁴, A¹, A², A³, A⁴ are as defined for the compound of formula (IA), or a salt or N-oxide thereof. The preferences for R³, R⁴, A¹, A², A³, A⁴ are the same as the preferences set out for the corresponding substituents of the compound of formula (IA).

Another group of compounds of formula (LIX) are compounds of (LIXc)

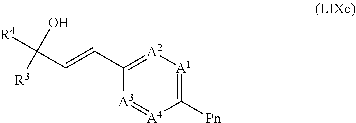

wherein A¹, A², A³, A⁴, R³ and R⁴ are as defined for the compounds of formula (IA), and Pn is a heterocyclyl or heteocyclyl substituted by one to five Z, as defined for compounds of formula (IA). The preferences for A¹, A², A³, A⁴, R³, R⁴ and heterocyclyl are the same as the preferences set out for the corresponding substituents of a compound of formula (IA).

Another group of novel intermediates are compounds of formula (LII)

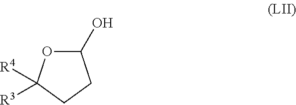

wherein R³ and R⁴ are as defined for compounds of formula (IA), or a salt or N-oxide thereof. The preferences for R³ and R⁴ are the same as the preferences set out for the corresponding substituents of a compound of formula (IA).

Another group of novel intermediates are compounds of formula (LIII)

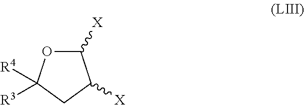

wherein R³ and R⁴ are as defined for compounds of formula (IA), and X represents Cl, Br or I, or a salt or N-oxide thereof. The preferences for R³ and R⁴ are the same as the preferences set out for the corresponding substituents of a compound of formula (IA).

Another group of novel intermediates are compounds of formula (XXVI)

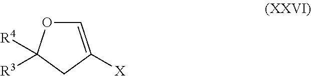

wherein R³ and R⁴ are as defined for the compound of formula (IA) and X is Cl, Br or I, or a salt or N-oxide thereof. The preferences for R³ and R⁴ are the same as the preferences set our for the corresponding substituents of the compound of formula (IA).

Another group of novel intermediates are compounds of formula (XXXVI)

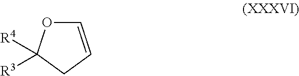

wherein R³ and R⁴ are as defined for the compound of formula (IA). The preferences for R³ and R⁴ are the same as the preferences set our for the corresponding substituents of the compound of formula (IA), or a salt or N-oxide thereof.

Another group of novel intermediates are compounds of formula (LI)

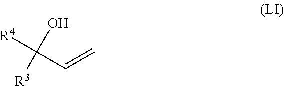

wherein R³ and R⁴ are as defined for the compound of formula (IA), or a salt or N-oxide thereof. The preferences for R³ and R⁴ are the same as the preferences set our for the corresponding substituents of the compound of formula (IA).

Additional intermediates are useful in the preparation of compounds as racemic mixtures of e.g. formula (LIX), and these compounds form additional aspects of the invention.

Another group of novel intermediates are compounds of formula (LV)

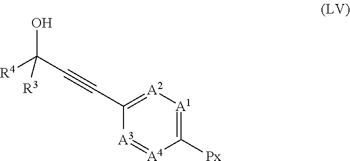

wherein R³, R⁴, A¹, A², A³ and A⁴ are as defined for compounds of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of a compound of formula (IA).

One group of compounds of formula (LV) are compounds of (LVa)

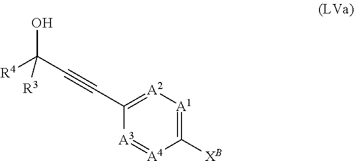

wherein R³, R⁴, A¹, A², A³, A⁴ are as defined for the compound of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of the compound of formula (IA).

Another group of compounds of formula (LVA) are compounds of (LVb)

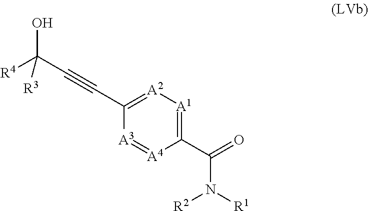

wherein R¹, R², R³, R⁴, A¹, A², A³, A⁴ are as defined for the compound of formula (IA), or a salt or N-oxide thereof. The preferences for R³, R⁴, A¹, A², A³, A⁴ are the same as the preferences set out for the corresponding substituents of the compound of formula (IA).

Another group of compounds of formula (LV) are compounds of (LVc)

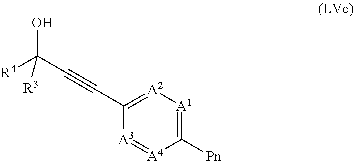

wherein A¹, A², A³, A⁴, R³ and R⁴ are as defined for the compounds of formula (IA), and Pn is a heterocyclyl or heteocyclyl substituted by one to five Z, as defined for compounds of formula I. The preferences for A¹, A², A³, A⁴, R³, R⁴ and heterocyclyl are the same as the preferences set out for the corresponding substituents of a compound of formula (IA).

The invention also provides compounds of formula (LVI)

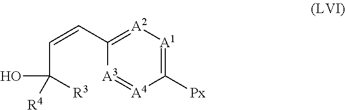

wherein R³, R⁴, A¹, A², A³ and A⁴ are as defined for compounds of formula (IA) and Px is as defined for the compound of formula (XXVA), or a salt or N-oxide thereof. The preferences for R³, R⁴, A¹, A², A³, A⁴ and Px (when Px is P) are the same as the preferences set out for the corresponding substituents of a compound of formula (IA).

One group of compounds of formula (LVI) are compounds of (LVIa)

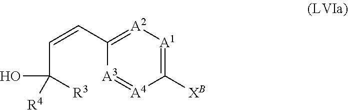

wherein R³, R⁴, A¹, A², A³ and A⁴ are as defined for compounds of formula (IA) and X^{B} is as defined for the compound of formula (XXVA), or a salt or N-oxide thereof. The preferences for R³, R⁴, A¹, A², A³, A⁴ are the same as the preferences set out for the corresponding substituents of a compound of formula (IA).

Another group of compounds of formula (LVI) are compounds of (LVIb)

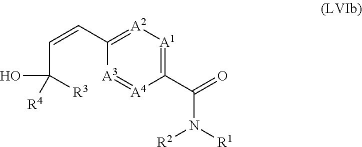

wherein R¹, R², R³, R⁴, A¹, A², A³ and A⁴ are as defined for compounds of formula (IA) and X^{B} is as defined for the compound of formula (XXVA), or a salt or N-oxide thereof. The preferences for R¹, R², R³, R⁴, A¹, A², A³, A⁴ are the same as the preferences set out for the corresponding substituents of a compound of formula (IA).

Another group of compounds of formula (LVI) are compounds of (LVIc)

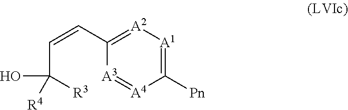

wherein A¹, A², A³, A⁴, R³ and R⁴ are as defined for the compounds of formula (IA), and Pn is a heterocyclyl or heteocyclyl substituted by one to five Z, as defined for compounds of formula I. The preferences for A¹, A², A³, A⁴, R³, R⁴ and heterocyclyl are the same as the preferences set out for the corresponding substituents of a compound of formula (IA).

The compounds of formula (IA) can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the compounds of the invention include those pests associated with agriculture (which term includes the growing of crops for food and fiber products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies). The compounds of the invention may be used for example on turf, ornamentals, such as flowers, shrubs, broad-leaved trees or evergreens, for example conifers, as well as for tree injection, pest management and the like. Compositions comprising the compound of formula (IA) may be used on ornamental garden plants (e.g. flowers, shrubs, broad-leaved trees or evergreens), e.g. to control aphids, whitefly, scales, meelybug, beetles and caterpillars. Compositions comprising the compound of formula (IA) may be used on garden plants (e.g. flowers, shrubs, broad-leaved trees or evergreens), on indoor plants (e.g. flowers and shrubs) and on indoor pest e.g. to control aphids, whitefly, scales, meelybug, beetles and caterpillars.

Furthermore, the compounds of the invention may be effective against harmful insects, without substantially imposing any harmful side effects to cultivated plants. Application of the compounds of the invention may increase the harvest yields, and may improve the quality of the harvested material. The compounds of the invention may have favourable properties with respect to amount appled, residue formulation, selectivity, toxicity, production methodology, high activity, wide spectrum of control, safety, control of resistant organisms, e.g. pests that are resistant to organic phosphorus agents and/or carbamate agents.

Examples of pest species which may be controlled by the compounds of formula (IA) include: coleopterans, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis; lepidopterans, for example, Lymantria dispar, Malacosoma neustria), Pieris rapae, Spodoptera litura, Mamestra brassicae, Chilo suppressalis), Pyrausta nubilalis, Ephestia cautella, Adoxophyes orana, Carpocapsa pomonella, Agrotisfucosa, Galleria mellonella, Plutella maculipennis, Heliothis virescens, Phyllocnistis citrella; hemipterans, for example, Nephotettix cincticeps, Nilaparvata lugens, Pseudococcus comstocki, Unaspis yanonensis, Myzus persicas, Aphis pomi, Aphis gossypii, Rhopalosiphum pseudobrassicas, Stephanitis nashi, Nezara spp., Trialeurodes vaporariorm, Psylla spp.; thysanopterans, for example, Thrips palmi, Franklinella occidental; orthopterans, for example, Blatella germanica, Periplaneta americana, Gryllotalpa Africana, Locusta migratoria migratoriodes; isopterans, for example, Reticulitermes speratus, Coptotermes formosanus; dipterans, for example, Musca domestica, Aedes aegypti, Hylemia platura, Culex pipiens, Anopheles sinensis, Culex tritaeniorhynchus, Liriomyza trifolii; acari, for example, Tetranychus cinnabarinus, Tetranychus urticae, Panonychus citri, Aculops pelekassi, Tarsonemus spp.; nematodes, for example, Meloidogyne incognita, Bursaphelenchus lignicolus Mamiya et Kiyohara, Aphelenchoides besseyi, Heterodera glycines, Pratylenchus spp.

Examples of further pest species which may be controlled by the compounds of formula (IA) include: from the order of the Anoplura (Phthiraptera), for example, Damalinia spp., Haematopinus spp., Linognathus spp., Pediculus spp., Trichodectes spp.; from the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici; from the class of the Bivalva, for example, Dreissena spp.; from the order of the Chilopoda, for example, Geophilus spp., Scutigera spp.; from the order of the Coleoptera, for example, Acanthoscehdes obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.; from the order of the Collembola, for example, Onychiurus armatus; from the order of the Dermaptera, for example, Forficula auricularia; from the order of the Diplopoda, for example, Blaniulus guttulatus; from the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.; from the class of the Gastropoda, for example, Anion spp., Biomphalaria spp., Bulinus spp., Deroceras spp., Galba spp., Lymnaea spp., Oncomelania spp., Succinea spp.; from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti; ft may be furthermore possible to control protozoa, such as Eimeria; from the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.; from the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma pini, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigena, Cercopidae, Cenoplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeunodes spp., Diaphorina spp., Diaspis spp., Dorsalis spp., Drosicha spp., Dysaphis spp., Dysmicoccus spp., Empoasca spp., Eriosoma spp., Erythnoneuna spp., Euscelis bilobatus, Geococcus coffeae, Homalodisca coagulata, Hyaloptenus arundinis, Icerya spp., Idiocerus spp., Idioscopus spp., Laodelphax stniatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Onthezia pnaelonga, Parabemisia myricae, Paratrioza spp., Panlatonia spp., Pemphigus spp., Peregrinus maidis, Phenacoccus spp., Phloeomyzus passeninii, Phorodon humuli, Phylloxera spp., Pinnaspis aspidistrae, Planococcus spp., Protopulvinania pyriformis, Pseudaulacaspis pentagona, Pseudococcus spp., Psylla spp., Pteromalus spp., Pynilla spp., Quadnaspidiotus spp., Quesada gigas, Rastrococcus spp., Rhopalosiphum spp., Saissetia spp., Scaphoides titanus, Schizaphis graminum, Selenaspidus anticulatus, Sogata spp., Sogatella furcifera, Sogatodes spp., Stictocephala festina, Tenalaphana malayensis, Tinocallis caryaefoliae, Tomaspis spp., Toxoptera spp., Trialeunodes vaporariorum, Trioza spp., Typhlocyba spp., Unaspis spp., Viteus vitifolii; from the order of the Hymenoptera, for example, Dipnion spp., Hoplocampa spp., Lasius spp., Mono-morium pharaonic, Vespa spp.; from the order of the Isopoda, for example, Armadillidium vulgane, Oniscus asellus, Poncellio scaber; from the order of the Isoptena, for example, Reticulitermes spp., Odontotermes spp.; from the order of the Lepidoptera, for example, Acronicta major, Aedia leucomelas, Agnotis spp., Alabama angillacea, Anticansia spp., Banathna brassicae, Bucculatnix thunbeniella, Bupalus pinianius, Cacoecia podana, Capua neticulana, Carpocapsa pomonella, Chematobia brumata, Chilo spp., Chonistoneuna fumiferana, Clysia ambiguella, Cnaphalocerus spp., Eanias insulana, Ephestia kuehniella, Euproctis chrysonnhoea, Euxoa spp., Feltia spp., Galleria mellonella, Helicovenpa spp., Heliothis spp., Hofmannophila pseudospretella, Homona magnanima, Hyponomeuta padella, Laphygma spp., Lithocolletis blancandella, Lithophane antennata, Loxagnotis albicosta, Lymantria spp., Malacosoma neustnia, Mamestra brassicae, Mocis nepanda, Mythimna sepanata, Onia spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.; from the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria; from the order of the Siphonaptera, for example, Ceratophyllus spp., Xenopsylla cheopis. From the order of the Symphyla, for example, Scutigerella immaculata; from the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.; from the order of the Thysanura, for example, Lepisma saccharina. The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

In particular, the compounds of the invention may be used to control the following pest species:

Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulfureus), Solenopsis geminata (fire ant), Monomorium pharaonic (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp. (citrus nematodes), Haemonchus contortus (barber pole worm), Caenorhabditis elegans (vinegar eelworm), Trichostrongylus spp. (gastro intestinal nematodes) and Deroceras reticulatum (slug).

The compound of formula (IA) may be used for pest control on various plants, including soybean (e.g. in some cases 10-70 g/ha), corn (e.g. in some cases 10-70 g/ha), sugarcane (e.g. in some cases 20-200 g/ha), alfalfa (e.g. in some cases 10-70 g/ha), brassicas (e.g. in some cases 10-50 g/ha), oilseed rape (e.g. canola) (e.g. in some cases 20-70 g/ha), potatoes (including sweet potatoes) (e.g. in some cases 10-70 g/ha), cotton (e.g. in some cases 10-70 g/ha), rice (e.g. in some cases 10-70 g/ha), coffee (e.g. in some cases 30-150 g/ha), citrus (e.g. in some cases 60-200 g/ha), almonds (e.g. in some cases 40-180 g/ha), fruiting vegetables, cucurbits and pulses (e.g. tomatoes, pepper, chili, eggplant, cucumber, squash etc.) (e.g. in some cases 10-80 g/ha), tea (e.g. in some cases 20-150 g/ha), bulb vegetables (e.g. onion, leek etc.) (e.g. in some cases 30-90 g/ha), grapes (e.g. in some cases 30-180 g/ha), pome fruit (e.g. apples, pears etc.) (e.g. in some cases 30-180 g/ha), and stone fruit (e.g. pears, plums etc.) (e.g. in some cases 30-180 g/ha).

The compounds of the invention may be used for pest control on various plants, including soybean, corn, sugarcane, alfalfa, brassicas, oilseed rape (e.g. canola), potatoes (including sweet potatoes), cotton, rice, coffee, citrus, almonds, fruiting vegetables, cucurbits and pulses (e.g. tomatoes, pepper, chili, eggplant, cucumber, squash etc.), tea, bulb vegetables (e.g. onion, leek etc.), grapes, pome fruit (e.g. apples, pears etc.), stone fruit (e.g. pears, plums etc.), and cereals.

The compounds of the invention may be used on soybean to control, for example, Elasmopalpus lignosellus, Diloboderus abderus, Diabrotica speciosa, Trialeurodes spp., Bemisia spp., aphids, Sternechus subsignatus, Formicidae, Agrotis ypsilon, Julus spp., Murgantia spp., Halyomorpha spp., Thyanta spp., Megascelis ssp., Procornitermes ssp., Gryllotalpidae, Nezara viridula, Piezodorus spp., Acrosternum spp., Neomegalotomus spp., Cerotoma trifurcata, Popillia japonica, Edessa spp., Liogenys fuscus, stalk borer, Scaptocoris castanea, phyllophaga spp., Migdolus spp., Pseudoplusia includens, Anticarsia gemmatalis, Epinotia spp., Rachiplusia spp., Spodoptera spp. (e.g. Spodoptera frugiperda), Bemisia tabaci, Tetranychus spp., Agriotes spp., Euschistus spp. (e.g. Euschistus heros). The compounds of the invention are preferably used on soybean to control Diloboderus abderus, Diabrotica speciosa, Trialeurodes spp., Bemisia spp., Nezara viridula, Piezodorus spp., Acrosternum spp., Cerotoma trifurcata, Popillia japonica, Euschistus heros, Scaptocoris castanea, phyllophaga spp., Migdolus spp., Agriotes spp., Euschistus spp.

The compounds of the invention may be used on corn to control, for example, Euschistus spp. (e.g. Euschistus heros), Dichelops furcatus, Diloboderus abderus, Thyanta spp., Elasmopalpus lignosellus, Halyomorpha spp., Spodoptera frugiperda, Nezara viridula, Cerotoma trifurcata, Popillia japonica, Agrotis ypsilon, Diabrotica speciosa, aphids, Heteroptera, Procornitermes spp., Scaptocoris castanea, Formicidae, Julus ssp., Dalbulus maidis, Diabrotica spp. (e.g. Diabrotica virgifera), Mocis latipes, Bemisia tabaci, heliothis spp., Tetranychus spp., thrips spp., phyllophaga spp., Migdolus spp., scaptocoris spp., Liogenys fuscus, Spodoptera spp., Ostrinia spp., Sesamia spp., wireworms, Agriotes spp., Halotydeus destructor. The compounds of the invention are preferably used on corn to control Euschistus spp., (e.g. Euschistus heros), Dichelops furcatus, Diloboderus abderus, Nezara viridula, Cerotoma trifurcata, Popillia japonica, Diabrotica spp. (e.g. Diabrotica speciosa, Diabrotica virgifera), Tetranychus spp., Thrips spp., Phyllophaga spp., Migdolus spp., Scaptocoris spp., Agriotes spp.

The compounds of the invention may be used on sugar cane to control, for example, Sphenophorus spp., termites, Migdolus spp., Diloboderus spp., Telchin licus, Diatrea saccharalis, Mahanarva spp., Mealybugs, Chilo spp.

The compounds of the invention may be used on alfalfa to control, for example, Hypera brunneipennis, Hypera postica, Colias eurytheme, Collops spp., Empoasca solana, Epitrix spp., Geocoris spp., Lygus hesperus, Lygus lineolaris, Spissistilus spp., Spodoptera spp., Aphids, Trichoplusia ni. The compounds of the invention are preferably used on alfalfa to control Hypera brunneipennis, Hypera postica, Empoasca solana, Epitrix spp., Lygus hesperus, Lygus lineolaris, Trichoplusia ni.

The compounds of the invention may be used on brassicas to control, for example, Chrysodeixis spp., Plutella xylostella, Pieris spp. (e.g. Pieris brassicae, Pieris rapae, Pieris napi), Mamestra spp. (e.g. Mamestra brassicae), Plusia spp., Trichoplusia spp. (e.g. Trichoplusia ni), Phyllotreta spp. (e.g. Phyllotreta cruciferae, Phyllotreta striolata), Spodoptera spp., Empoasca spp., thrips spp., Delia spp., Murgantia spp., Trialeurodes spp., Bemisia spp., Microtheca spp., Aphids, Chaetocnema spp., Psylliodes spp. (e.g. Psylliodes chrysocephala). The compounds of the invention are preferably used on brassicas to control Plutella xylostella, Pieris spp., Plusia spp., Trichoplusia ni, Phyllotreta spp., Thrips spp., Chaetocnema spp.

The compounds of the invention may be used on oil seed rape, e.g. canola, to control, for example, Meligethes spp. (e.g. Meligethes aeneus), Ceutorhynchus spp., (e.g. Ceutorhynchus assimilis, Ceutorhynchus napi), Halotydeus destructor, Psylloides spp. (e.g. Psylliodes chrysocephala), Phyllotreta spp. (e.g. Phyllotreta cruciferae, Phyllotreta striolata), Chaetocnema spp.

The compounds of the invention may be used on potatoes, including sweet potatoes, to control, for example, Empoasca spp., Leptinotarsa spp., Diabrotica speciosa, Phthorimaea spp., Paratrioza spp., Maladera matrida, Agriotes spp., Aphids, wireworms. The compounds of the invention are preferably used on potatoes, including sweet potatoes, to control Empoasca spp., Leptinotarsa spp., Diabrotica speciosa, Phthorimaea spp., Paratrioza spp., Agriotes spp.

The compounds of the invention may be used on cotton to control, for example, Anthonomus grandis, Pectinophora spp., heliothis spp., Spodoptera spp., Tetranychus spp. (e.g. Tetranychus urticae), Empoasca spp., Thrips spp. (e.g. Thrips tabaci, Thrips palmi), Bemisia tabaci, Trialeurodes spp., Aphids, Lygus spp. (e.g. Lygus lineolaris, Lygus Hesperus), phyllophaga spp., Scaptocoris spp., Austroasca viridigrisea, Creontiades spp., Nezara spp., Piezodorus spp., Halotydeus destructor, Oxycaraenus hyalinipennis, Dysdercus cingulatus, Amrasca spp. (e.g. Amrasca biguttula biguttula), Frankliniella spp. (e.g. Frankliniella schultzei), Scirtothrips spp. (e.g. Scirtothrips dorsali), Anaphothrips spp., Polyphagotarsonemus latus. The compounds of the invention are preferably used on cotton to control Anthonomus grandis, Tetranychus spp., Empoasca spp., thrips spp., Lygus spp., phyllophaga spp., Scaptocoris spp.

The compounds of the invention may be used on rice to control, for example, Leptocorisa spp. (e.g. Leptocorisa oratorius, Leptocorisa chinensis, Leptocorisa acuta), Cnaphalocrosis spp., Chilo spp. (e.g. Chilo suppressalis, Chilo polychrysus, Chilo auricilius), Scirpophaga spp. (e.g. Scirpophaga incertulas, Scirpophaga innotata, Scirpophaga nivella), Lissorhoptrus spp., Oebalus pugnax, Scotinophara spp. (e.g. Scotinophara coarctata, Scotinophara lurida, Scotinophara latiuscula), Nephotettix spp. (e.g. Nephotettix malayanus, Nephotettix nigropictus, Nephotettix parvus, Nephottetix virescens, Nephotettix cincticeps), Mealybugs, Sogatella furcifera, Nilaparvata lugens, Orseolia spp. (e.g. Orseolia oryzae), Cnaphalocrocis medinalis, Marasmia spp. (e.g. Marasmia patnalis, Marasmia exigua), Stenchaetothrips biformis, Thrips spp., Hydrellia spp. (e.g. Hydrellia philippina), Grasshoppers, Pomacea canaliculata, Scirpophaga innotata, Sesamia inferens, Laodelphax striatellus, Nymphula depunctalis, Oulema oryzae, Stinkbugs. The compounds of the invention are preferably used on rice to control Leptocorisa spp., Lissorhoptrus spp., Oebalus pugnax, Nephotettix spp. (e.g. Nephotettix malayanus, Nephotettix nigropictus, Nephotettix parvus, Nephottetix virescens, Nephotettix cincticeps), Sogatella furcifera, Stenchaetothrips biformis, Thrips spp., Hydrellia spp. (e.g. Hydrellia philippina), Grasshoppers, Pomacea canaliculata, Scirpophaga innotata, Chilo spp., Oulema oryzae.

The compounds of the invention may be used on coffee to control, for example, Hypothenemus spp. (e.g. Hypothenemus Hampei), Perileucoptera Coffeella, Tetranychus spp., Brevipalpus spp., Mealybugs. The compounds of the invention are preferably used on coffee to control Hypothenemus Hampei, Perileucoptera Coffeella.

The compounds of the invention may be used on citrus to control, for example, Panonychus citri, Phyllocoptruta oleivora, Brevipalpus spp. (e.g. Brevipalpus californicus, Brevipalpus phoenicis), Diaphorina citri, Scirtothrips spp. (e.g. Scirtothrips dorsalis), Thrips spp., Unaspis spp., Ceratitis capitata, Phyllocnistis spp. (e.g. Phyllocnistis citrella), Aphids, Hardscales, Softscales, Mealybugs. The compounds of the invention are preferably used on citrus to control Panonychus citri, Phyllocoptruta oleivora, Brevipalpus spp., Diaphorina citri, Scirtothrips spp., thrips spp., Phyllocnistis spp.

The compounds of the invention may be used on almonds to control, for example, Amyelois transitella, Tetranychus spp.

The compounds of the invention may be used on fruiting vegetables, cucurbits and pulses, including tomatoes, pepper, chili, eggplant, cucumber, squash etc., to control, for example, Thrips spp., Tetranychus spp. (e.g. Tetranychus urticae)., Polyphagotarsonemus spp. (e.g. Polyphagotarsonemus latus), Aculops spp. (e.g. Aculops lycopersici), Empoasca spp. (e.g. Empoasca fabae), Spodoptera spp., heliothis spp., Tuta absoluta, Liriomyza spp. (e.g. Liriomyza brassicae, Liriomyza bryoniae, Liriomyza huidobrensis, Liriomyza sativae, Liriomyza trifolii), Bemisia tabaci, Trialeurodes spp., Aphids, Paratrioza spp., Frankliniella spp. (e.g. Frankliniella occidentalis, Frankliniella intonsa, Frankliniella bispinosa), Spodoptera spp. (e.g. Spodoptera exigua, Spodoptera littoralis, Spodoptera litura, Spodoptera frugiperda, Spodoptera eridania), Anthonomus spp., Phyllotreta spp., Amrasca spp. (e.g. Amrasca biguttula biguttula), Epilachna spp., Halyomorpha spp., Scirtothrips spp., Leucinodes spp. (e.g. Leucinodes orbonalis), Neoleucinodes spp. (e.g. Neoleucinodes elegantalis), Maruca spp., Fruit flies, Stinkbugs, Lepidopteras, Coleopteras, Helicoverpa spp. (e.g. Helicoverpa armigera), Heliothis spp. (e.g. Heliothis virescens), Paratrioza spp. (e.g. Paratrioza cockerelli), The compounds of the invention are preferably used on fruiting vegetables, cucurbits and pulses, including tomatoes, pepper, chili, eggplant, cucumber, squash etc., to control Thrips spp., Tetranychus spp., Polyphagotarsonemus spp., Aculops spp., Empoasca spp., Spodoptera spp., heliothis spp., Tuta absoluta, Liriomyza spp., Paratrioza spp., Frankliniella occidentalis, Frankliniella spp., Amrasca spp., Scirtothrips spp., Leucinodes spp., Neoleucinodes spp.

The compounds of the invention may be used on tea to control, for example, Pseudaulacaspis spp., Empoasca spp., Scirtothrips spp., Caloptilia theivora, Tetranychus spp. The compounds of the invention are preferably used on tea to control Empoasca spp., Scirtothrips spp.

The compounds of the invention may be used on bulb vegetables, including onion, leek etc. to control, for example, Thrips spp., Spodoptera spp., heliothis spp. The compounds of the invention are preferably used on bulb vegetables, including onion, leek etc. to control Thrips spp.

The compounds of the invention may be used on grapes to control, for example, Empoasca spp., Lobesia spp., Eupoecilia ambiguella, Frankliniella spp., Thrips spp., Tetranychus spp., Rhipiphorothrips Cruentatus, Eotetranychus Willamettei, Erythroneura Elegantula, Scaphoides spp., Scelodonta strigicollis, Mealybugs. The compounds of the invention are preferably used on grapes to control Frankliniella spp., Thrips spp., Tetranychus spp., Rhipiphorothrips Cruentatus, Scaphoides spp.

The compounds of the invention may be used on pome fruit, including apples, pears etc., to control, for example, Cacopsylla spp., Psylla spp., Panonychus ulmi, Cydia pomonella, Lepidopteras, Aphids, Hardscales, Softscales. The compounds of the invention are preferably used on pome fruit, including apples, pears etc., to control Cacopsylla spp., Psylla spp., Panonychus ulmi.

The compounds of the invention may be used on stone fruit to control, for example, Grapholita molesta, Scirtothrips spp., Thrips spp., Frankliniella spp., Tetranychus spp., Aphids, Hardscales, Softscales, Mealybugs. The compounds of the invention are preferably used on stone fruit to control Scirtothrips spp., Thrips spp., Frankliniella spp., Tetranychus spp.

The compounds of the invention may be used on cereals to control, for example, Aphids, Stinkbugs, earthmites, Eurygaster integriceps, Zabrus tenebrioides, Anisoplia austriaca, Chaetocnema aridula, Phyllotreta spp., Oulema melanopus, Oscinella spp., Delia spp., Mayetiola spp., Contarinia spp., Cephus spp., Steneotarsonemus spp., Apamea spp.

In another embodiment compounds of formula (IA) and mixtures of the invention may be used on rice to control Baliothrips biformis (Thrips), Chilo spp. (e.g. Chilo polychrysus (Dark headed striped borer), Chilo suppressalis (Rice stemborer), Chilo indicus (Paddy stem borer), Chilo polychrysus (Dark-headed rice borer), Chilo suppressalis (Stripe stem borer)), Cnaphalocrocis medinalis (Rice leaf folder), Dicladispa armigera (Hispa), Hydrellia philipina (Rice whorl-maggot), Laodelphax spp. (Smaller brown planthopper) (e.g. Laodelphax striatellus), Lema oryzae (Rice leafbeetle), Leptocorsia acuta (Rice bug), Leptocorsia oratorius (rice bug), Lissorhoptrus oryzophilus (rice water weevil), Mythemina separata (armyworm), Nephottetix spp. (Green leafhopper) (e.g. Nephotettix cincticeps, Nephotettix malayanus, Nephotettix nigropictus, Nephotettix parvus, Nephottetix virescens), Nilaparvata lugens (Brown Planthopper), Nymphula depunctalis (Rice caseworm), Orseolia oryzae (Rice Gall midge), Oulema oryzae (Rice leafbeetle), Scirpophaga incertulas (Yellow Stemborer), Scirpophaga innotata (White Stemborer), Scotinophara coarctata (Rice black bug), Sogaella frucifera (White-backed planthopper), Steneotarsonemus spinki.

The compounds of the invention may be used to control animal housing pests including: Ants, Bedbugs (adult), Bees, Beetles, Boxelder Bugs, Carpenter Bees, Carpet Beetles, Centipedes, Cigarette, Beetles, Clover Mites, Cockroaches, Confused Flour Beetle, Crickets, Earwigs, Firebrats, Fleas, Flies, Lesser Grain Borers, Millipedes, Mosquitoes, Red Flour Beetles, Rice Weevils, Saw-toothed Grain Beetles, Silverfish, Sowbugs, Spiders, Termites, Ticks, Wasps, Cockroaches, Crickets, Flies, Litter Beetles (such as Darkling, Hide, and Carrion), Mosquitoes, Pillbugs, Scorpions, Spiders, Spider Mites (Twospotted, Spruce), Ticks.

The compounds of the invention may be used to control ornamental pests including: Ants (Including Imported fire ants), Armyworms, Azalea caterpillars, Aphids, Bagworms, Black vine weevils (adult), Boxelder bugs, Budworms, California oakworms, Cankerworms, Cockroaches, Crickets, Cutworms, Eastern tent caterpillars, Elm leaf beetles, European sawflies, Fall webworms, Flea beetles, Forest tent caterpillars, Gypsy moth larvae, Japanese beetles (adults), June beetles (adults), Lace bugs, Leaf-feeding caterpillars, Leafhoppers, Leafminers (adults), Leaf rollers, Leaf skeletonizers, Midges, Mosquitoes, Oleander moth larvae, Pillbugs, Pine sawflies, Pine shoot beetles, Pinetip moths, Plant bugs, Root weevils, Sawflies, Scale insects (crawlers), Spiders, Spittlebugs, Striped beetles, Striped oakworms, Thrips, Tip moths, Tussock moth larvae, Wasps, Broadmites, Brown softscales, California redscales (crawlers), Clover mites, Mealybugs, Pineneedlescales (crawlers), Spider mites, Whiteflies

The compounds of the invention may be used to control turf pests including: Ants (Including Imported fire ants, Armyworms, Centipedes, Crickets, Cutworms, Earwigs, Fleas (adult), Grasshoppers, Japanese beetles (adult), Millipedes, Mites, Mosquitoes (adult), Pillbugs, Sod webworms, Sow bugs, Ticks (including species which transmit Lyme disease), Bluegrass billbugs (adult), Black turfgrass ataenius (adult), Chiggers, Fleas (adult), Grubs (suppression), Hyperodes weevils (adult), Mole crickets (nymphs and young adults), Mole Crickets (mature adults), Chinch Bugs.

The compounds of formula (IA) and mixture of the invention, in particular those in the tables above, may be used for soil applications, including as a seed application, to target at least the following: sucking pests such as aphids, thrips, brown plant hopper (e.g. on rice), sting bugs, white flies (e.g. on cotton and vegetables), mites; on soil pests such as corn root worm, wireworms, white grubs, zabrus, termites (e.g. on sugar cane, soy, pasture), maggots, cabbage root fly, red legged earth mite; on lepidoptera, such as spodoptera, cutworms, elasmoplpus, plutella (e.g. brassica), stem borers, leaf miners, flea beetle, Sternechus; on nematicides, such as Heterodera glycines (e.g. on soybean), Pratylenchus brachyurus (e.g. on corn), P. zeae (e.g. oncorn), P. penetrans (e.g. on corn), Meloidogyne incognita (e.g. on vegetables), Heterodera schachtii (e.g. on sugar beet), Rotylenchus reniformis (e.g. on cotton), Heterodera avenae (e.g. on cereals), Pratylenchus neglectus (e.g. on cereals), thornei (e.g. on cereals).

The compounds of formula (IA) and mixture of the invention, in particular those in the tables above may be used for seed applications at least on the following: soil grubs for corn, soybeans, sugarcane: Migdolus spp; Phyllophaga spp.; Diloboderus spp; Cyclocephala spp; Lyogenys fuscus; sugarcane weevils: Sphenophorus levis & Metamasius hemipterus; termites for soybeans, sugarcane, pasture, others: Heterotermes tenuis; Heterotermes longiceps; Cornitermes cumulans; Procornitermes triacifer; Neocapritermes opacus; Neocapritermes parvus; corn root worms for corn and potatoes: Diabrotica spp., seed Maggot: Delia platura; soil stinkbugs: Scaptocoris castanea; wireworms: Agriotes spp; Athous spp Hipnodes bicolor; Ctenicera destructor; Limonius canu; Limonius californicus; rice water weevil: Lissorhoptrus oryzophilus; Red Legged earth mites: Halotydeus destructor.

The invention therefore provides a method of combating and/or controlling an animal pest, e.g. an invertebrate animal pest, which comprises applying to the pest, to a locus of the pest, or to a plant susceptible to attack by the pest a pesticidally effective amount of a compound of formula (IA). In particular, the invention provides a method of combating and/or controlling insects, acarines, nematodes or molluscs which comprises applying an insecticidally, acaricidally, nematicidally or molluscicidally effective amount of a compound of formula (IA), or a composition containing a compound of formula (IA), to a pest, a locus of pest, preferably a plant, or to a plant susceptible to attack by a pest, The compounds of formula (IA) are preferably used against insects, acarines or nematodes.

The term “plant” as used herein includes seedlings, bushes and trees. Crops are to be understood as also including those crops which have been rendered tolerant to herbicides or classes of herbicides (e.g. ALS-, GS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

The compounds of the invention may be applied to plant parts. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds. Treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seed, also by applying one or more coats.

Compounds of formula (IA) may be used on transgenic plants (including cultivars) obtained by genetic engineering methods and/or by conventional methods. These are understood as meaning plants having novel properties (“traits”) which have been obtained by conventional breeding, by mutagenesis or by recombinant DNA techniques. Depending on the plant species or plant cultivars, their location and growth conditions (soils, climate, vegetation period, diet), the treatment according to the invention may also result in superadditive “synergistic”) effects.

Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products.

Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds.

Examples of transgenic plants which may be mentioned are the important crop plants, such as cereals (wheat, rice), maize, soybean, potatoes, sugar beet, tomatoes, peas and other vegetable varieties, cotton, tobacco, oilseed rape and also fruit plants (with the fruits apples, pears, citrus fruits and grapes).

Compounds of formula (IA) may be used on transgenic plants that are capable of producing one or more pesticidal proteins which confer upon the transgenic plant tolerance or resistance to harmful pests, e.g. insect pests, nematode pests and the like. Such pesticidal proteins include, without limitation, Cry proteins from Bacillus thuringiensis Cry1Ab, Cry1Ac, Cry1F, Cry2Ab, Cry2Ae, Cry3A, Cry3Bb, or Cry9C; engineered proteins such as modified Cry3A (U.S. Pat. No. 7,030,295) or Cry1A.105; or vegetative insecticidal proteins such as Vip1, Vip2 or Vip3. A full list of Bt Cry proteins and VIPs useful in the invention can be found on the worldwide web at Bacillus thuringiensis Toxin Nomenclature Database maintained by the University of Sussex (see also, Crickmore et al. (1998) Microbiol. Mol. Biol. Rev. 62:807-813). Other pesticidal proteins useful in the invention include proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. Further examples of such pesticidal proteins or transgenic plants capable of synthesizing such proteins are disclosed, e.g., in EP-A 374753, WO 93/007278, WO 95/34656, EP-A 427529, EP-A 451878, WO 03/18810 and WO 03/52073. The methods for producing such transgenic plants are generally known to the person skilled in the art and some of which are commercially available such as Agrisure®CB (P1) (corn producing Cry1Ab), Agrisure®RW (P2) (corn producing mCry3A), Agrisure® Viptera (P3) (corn hybrids producing Vip3Aa); Agrisure300GT (P4) (corn hybrids producing Cry1Ab and mCry3A); YieldGard® (P5) (corn hybrids producing the Cry1Ab protein), YieldGard® Plus (P6) (corn hybrids producing Cry1Ab and Cry3Bb1), Genuity® SmartStax® (P7) (corn hybrids with Cry1A.105, Cry2Ab2, Cry1F, Cry34/35, Cry3Bb); Herculex® I (P8) (corn hybrids producing Cry1Fa) and Herculex®RW (P9) (corn hybrids producing Cry34Ab1, Cry35Ab1 and the enzyme Phosphinothricin-N-Acetyltransferase [PAT]); NuCOTN®33B (P10) (cotton cultivars producing Cry1Ac), Bollgard®I (P11) (cotton cultivars producing Cry1Ac), Bollgard®II (P12) (cotton cultivars producing Cry1Ac and Cry2Ab2) and VIPCOT® (P13) (cotton cultivars producing a Vip3Aa). Soybean Cyst Nematode resistance soybean (SCN®-Syngenta (P14)) and soybean with Aphid resistant trait (AMT® (P15)) are also of interest.

Further examples of such transgenic crops are:

1. Bt11 Maize from Syngenta Seeds SAS, Chemin de l'Hobit 27, F-31 790 St. Sauveur, France, registration number C/FR/96/05/10 (P16). Genetically modified Zea mays which has been rendered resistant to attack by the European corn borer (Ostrinia nubilalis and Sesamia nonagrioides) by transgenic expression of a truncated CryIA(b) toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10 (P17). Genetically modified Zea mays which has been rendered resistant to attack by the European corn borer (Ostrinia nubilalis and Sesamia nonagrioides) by transgenic expression of a CryIA(b) toxin. Bt176 maize also transgenically expresses the enzyme PAT to achieve tolerance to the herbicide glufosinate ammonium.

3. MIR604Maize from Syngenta Seeds SAS, Chemin de l'Hobit 27, F-31 790 St. Sauveur, France, registration number C/FR/96/05/10 (P18). Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9 (P19). MON 863 expresses a CryIIIB(b1) toxin and has resistance to certain Coleoptera insects.

5. IPC 531 Cotton from Monsanto Europe S.A. 270-272 Avenue de Tervuren, B-1150 Brussels, Belgium, registration number C/ES/96/02. (P20)

6. 1507 Maize from Pioneer Overseas Corporation, Avenue Tedesco, 7 B-1160 Brussels, Belgium, registration number C/NL/00/10. (P21) Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03 (P22). Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, include the European corn borer.

Further examples of transgenic plants, and of very high interest, are those carrying traits conferring resistance to 2.4D (e.g. Enlist®) (e.g. WO 2011066384) (P23), glyphosate (e.g. Roundup Ready® (P24), Roundup Ready 2 Yield® (P25)), sulfonylurea (e.g. STS®) (P26), glufosinate (e.g. Liberty Link® (P27), Ignite® (P28)), Dicamba (P29) (Monsanto), HPPD tolerance (P30) (e.g. isoxaflutole herbicide) (Bayer CropScience, Syngenta). Double or triple stacks of any of the traits described here are also of interest, including glyphosate and sulfonyl-urea tolerance ((e.g. Optimum GAT®) (P31), plants stacked with STS® and Roundup Ready® (P32) or plants stacked with STS® and Roundup Ready 2 Yield® (P33)), dicamba and glyphosate tolerance (P34) (Monsanto). Of particular interest are soybean plants carrying trains conferring resistance to 2.4D (e.g. Enlist®), glyphosate (e.g. Roundup Ready®, Roundup Ready 2 Yield®), sulfonylurea (e.g. STS®), glufosinate (e.g. Liberty Link®, Ignite®), Dicamba (Monsanto) HPPD tolerance (e.g. isoxaflutole herbicide) (Bayer CropScience, Syngenta). Double or triple stack in soybean plants of any of the traits described here are also of interest, including glyphosate and sulfonyl-urea tolerance (e.g. Optimum GAT®, plants stacked with STS® and Roundup Ready® or Roundup Ready 2 Yield®), dicamba and glyphosate tolerance (Monsanto). Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

Examples of cotton transgenic events include MON 531/757/1076 (Bollgard I®-Monsanto), MON1445 (Roundup Ready Cotton®-Monsanto), MON531×MON1445 (Bollgard I+RR®-Monsanto), MON15985 (Genuity Bollgard II Cotton®-Monsanto), MON88913 (Genuity RR FLEX Cotton®-Monsanto), MON15985×MON1445 (Genuity Bollgard II+RR FELX Cotton®-Monsanto), MON15983×MON88913 (Genuity Bollgard II+RR FLEX Cotton®-Monsanto), MON15985 (FibreMax Bollgard II Cotton®-Monsanto), LL25 (FibreMax LL Cotton®-BCS Stoneville), GHB614 (FibreMax GlyTol Cotton®-BCS Stoneville), LL25×MON15985 (FibreMax LL Bollgard II Cotton®-BCS Stoneville/Monsanto), GHB614×LL25 (FibreMax LL GlyTol Cotton®-BCS Stoneville), GHB614×LL25×MON15985 (FibreMax RR GlyTol Bollgard II Cotton®-BCS Stoneville), MON88913×MON15985 (FibreMax LL GlyTol Bollgard II Cotton®-Monsanto), MON88913 (FibreMax RR Flex Cotton®-Monsanto), GHB119+T304-40 (Twinlink®-BCS Stoneville), GHB119+T304-40×LL25×GHB614 (Twinlink LL GT®-BCS Stoneville), 3006-210-23×281-24-236 (PhytoGen Widestrike Insect Protection®-Dow), 3006-210-23×281-24-236×MON88913 (PhytoGen Widestrike Insect Protection®RR FLEX-® Dow/Monsanto), 3006-210-23×281-24-236×MON1445 ((PhytoGen Widestrike Insect Protection+RR®-Dow/Monsanto), MON1445 (PhytoGen Roundup Ready®-Monsanto), MON88913 (PhytoGen Roundup Ready FLEX®-Monsanto), COT102×COT67B (Vipcot®-Syngenta), COT102×COT67B×MON88913 (Vipcot RR FLEX®-Syngenta/Monsanto), 281-24-236 (Dow), 3006-210-23 (Dow), COT102 (Syngenta), COT67B (Syngenta), T304-40 (BCS Stoneville).

Examples of Soy transgenic events include MON87701×MON89788 (Genuity Roundup ready 2 Yield soybeans®-Monsanto), MON89788 (Roundup Ready2Yield®, RR2Y®-Monsanto), MON87708 (Monsanto), 40-3-2 (Roundup Ready®, RR1®-Monsanto), MON87701 (Monsanto), DAS-68416 (Enlist Weed Control System®-Dow), DP356043 (Optimum GAT®-Pioneer), A5547-127 (LibertyLink Soybean®-Bayercropscience), A2704-12 (Bayercropscience), GU262 (Bayercropscience), W62 W98 (Bayercropscience), CRV127 (Cultivance®-BASF/EMBRAPA).

Examples of Maize transgenic events include T25 (LibertyLink®, LL®-Bayerscropscience), DHT-1 (Dow), TC1507 (Herculex I®-Dow), DAS59122-7 (Herculex RW®-Dow), TC1507+DAS59122-7-Herculex Xtra®-Dow), TC1507×DAS-59122-7×NK603 (Herculex Xtra+RR®-Dow), TC1507×DAS-59122-×MON88017×MON89034 (Genuity Smartstax Corn®, Genuity Smartstax RIB complete®-Monsanto/Dow), MON89034×NK603 (Genuity VT double PRO®-Monsanto), MON89034+MON88017 (Genuity VT Triple PRO®-Monsanto), NK603 (Roundup Ready 2®, RR2®-Monsanto), MON810 (YieldGard BT®, Yieldgard cornborer®-Monsanto), MON810×NK603 (YieldGard cornborer RR Corn 2®-Monasnto), MON810×MON863 (YieldGard Plus®-Monsanto), MON863×MON810×NK603 (YieldGard Plus+RR Corn2®/YieldGard RR Maize®-Monsanto), MON863×NK603 (YieldGard Rotworm+RR Corn 2®-Monsanto), MON863 (YieldBard RW®-Monsanto), MON89034 (YieldGard RW®-Monsanto), MON88017 (YieldGard VT RW®-Monsanto), MON810+MON88017 (YieldGard VT Triple®-Monsanto), MON88017+MON89034 (YieldGard VT Triple Pro®-Monsanto), Bt11+MIR604+GA21 (Agrisure 3000®-Syngenta), Bt11+TC1507+MIR604+5307+GA21 (Syngenta), Bt11+TC1507+MIR604+DAS59122+GA21 (Agrisure 3122®-Syngenta), BT11 (Agrisure CB®-Syngenta), GA21-(Agrisure GT®-Syngenta), MIR604 (Agrisure RW®-Syngenta), Bt11+MIR162 (Agrisure TL VIP®-Syngenta), BT11+MIR162+GA21 (Agrisure Viptra 3110®-Syngenta), BT11+MIR162+MIR604 (Agrisure™ 3100®-Syngenta), Event3272+BT11+MIR604+GA21 (Syngenta), BT11+MIR1692+MIR604+GA21 (Agrisure Viptera 3111®-Syngenta), BT11+MIR 162+TC1507+GA21 (Agrisure Viptera 3220®-Syngenta), BT11+MIR162+TC1507+MIR604+5307+GA21 (Agrisure Viptera 3222®-Syngenta), MIR162 (Syngenta), BT11+GA21+MIR162+MIR604+5307 (Syngenta), 5307 (Syngenta).

In order to apply a compound of formula (IA) as an insecticide, acaricide, nematicide or molluscicide to a pest, a locus of pest, or to a plant susceptible to attack by a pest, a compound of formula (IA) is usually formulated into a composition which includes, in addition to the compound of formula (IA), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (IA). The composition is generally used for the control of pests such that a compound of formula (IA) is applied at a rate of from 0.1 g to 10 kg per hectare, preferably from 1 g to 6 kg per hectare, more preferably from 1 g to 1 kg per hectare.

When used in a seed dressing, a compound of formula (IA) is generally used at a rate of 0.0001 g to 10 g (for example 0.001 g or 0.05 g), preferably 0.005 g to 10 g, more preferably 0.005 g to 4 g, per kilogram of seed.

In another aspect the present invention provides a composition comprising a pesticidally effective amount of a compound of formula (IA), in particular an insecticidal, acaricidal, nematicidal or molluscicidal composition comprising an insecticidally, acaricidally, nematicidally or molluscicidally effective amount of a compound of formula (IA) and a suitable carrier or diluent therefor. The composition is preferably an insecticidal, acaricidal, nematicidal or molluscicidal composition.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (IA).

Dustable powders (DP) may be prepared by mixing a compound of formula (IA) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of formula (IA) with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (IA) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (IA) and one or more powdered solid diluents or carriers, or from pre-formed blank granules by absorbing a compound of formula (IA) (or a solution thereof, in a suitable agent) in a porous granular material (such as pumice, attapulgite clays, fuller's earth, kieselguhr, diatomaceous earths or ground corn cobs) or by adsorbing a compound of formula (IA) (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (IA) in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a compound of formula (IA) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (IA) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (IA) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (IA). SCs may be prepared by ball or bead milling the solid compound of formula (IA) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (IA) may be dry milled and added to water, containing agents hereinbefore described, to produce the desired end product.

Aerosol formulations comprise a compound of formula (IA) and a suitable propellant (for example n-butane). A compound of formula (IA) may also be dissolved or dispersed in a suitable medium (for example water or a water miscible liquid, such as n-propanol) to provide compositions for use in non-pressurized, hand-actuated spray pumps.

A compound of formula (IA) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a compound of formula (IA) and, optionally, a carrier or diluent therefor. The polymeric shell may be produced by either an interfacial polycondensation reaction or by a coacervation procedure. The compositions may provide for controlled release of the compound of formula (IA) and they may be used for seed treatment. A compound of formula (IA) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (IA)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (IA).

A compound of formula (IA) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (IA) may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of formula (IA) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (IA) (for example 0.0001 to 10%, by weight) depending upon the purpose for which they are to be used.

A compound of formula (IA) may be used in mixtures with fertilizers (for example nitrogen-, potassium- or phosphorus-containing fertilizers). Suitable formulation types include granules of fertilizer. The mixtures preferably contain up to 25% by weight of the compound of formula (IA).

The invention therefore also provides a fertilizer composition comprising a fertilizer and a compound of formula (IA).

The compositions of this invention may contain other compounds having biological activity, for example micronutrients or compounds having fungicidal activity or which possess plant growth regulating, herbicidal, insecticidal, nematicidal or acaricidal activity.

The compound of formula (IA) may be the sole active ingredient of the composition or it may be admixed with one or more additional active ingredients such as a pesticide, e.g. a insecticide, fungicide or herbicide, or a synergist or plant growth regulator where appropriate. An additional active ingredient may provide a composition having a broader spectrum of activity or increased persistence at a locus; synergize the activity or complement the activity (for example by increasing the speed of effect or overcoming repellency) of the compound of formula (IA); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin and gamma cyhalothrin), bifenthrin, fenpropathrin, cyfluthrin, tefluthrin, fish safe pyrethroids (for example ethofenprox), natural pyrethrin, tetramethrin, S-bioallethrin, fenfluthrin, prallethrin, acrinathirin, etofenprox or 5-benzyl-3-furylmethyl-(E)-(1R,3S)-2,2-dimethyl-3-(2-oxothiolan-3-ylidenemethyl)cyclopropane carboxylate;
b) Organophosphates, such as profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron, diafenthiuron, lufeneron, novaluron, noviflumuron or chlorfluazuron;
e) Organic tin compounds, such as cyhexatin, fenbutatin oxide or azocyclotin;
f) Pyrazoles, such as tebufenpyrad, tolfenpyrad, ethiprole, pyriprole, fipronil, and fenpyroximate;
g) Macrolides, such as avermectins or milbemycins, for example abamectin, emamectin benzoate, ivermectin, milbemycin, spinosad, azadirachtin, milbemectin, lepimectin or spinetoram;
h) Hormones or pheromones;
i) Organochlorine compounds, such as endosulfan (in particular alpha-endosulfan), benzene hexachloride, DDT, chlordane or dieldrin;
j) Amidines, such as chlordimeform or amitraz;
k) Fumigant agents, such as chloropicrin, dichloropropane, methyl bromide or metam;
l) Neonicotinoid compounds, such as imidacloprid, thiacloprid, acetamiprid, nitenpyram, dinotefuran, thiamethoxam, clothianidin, or nithiazine;
m) Diacylhydrazines, such as tebufenozide, chromafenozide or methoxyfenozide;
n) Diphenyl ethers, such as diofenolan or pyriproxifen;
o) Pyrazolines such as Indoxacarb or metaflumizone;
p) Ketoenols, such as Spirotetramat, spirodiclofen or spiromesifen;
q) Diamides, such as flubendiamide, chlorantraniliprole (Rynaxypyr®) or cyantraniliprole;
r) Essential oils such as Bugoil®-(PlantImpact); or
s) a compound selected from buprofezine, flonicamid, acequinocyl, bifenazate, cyenopyrafen, cyflumetofen, etoxazole, flometoquin, fluacrypyrim, fluensulfone, flufenerim, flupyradifuone, harpin, iodomethane, dodecadienol, pyridaben, pyridalyl, pyrimidifen, flupyradifurone, 4-[(6-Chloro-pyridin-3-ylmethyl)-(2,2-difluoro-ethyl)-amino]-5H-furan-2-one (DE 102006015467), CAS: 915972-17-7 (WO 2006129714; WO2011/147953; WO2011/147952), CAS: 26914-55-8 (WO 2007020986), chlorfenapyr, pymetrozine, sulfoxaflor and pyrifluqinazon.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704) (e.g. acibenzolar-5-methyl), alanycarb, aldimorph, anilazine, azaconazole, azoxystrobin, benalaxyl, benomyl, benthiavalicarb, biloxazol, bitertanol, bixafen, blasticidin S, boscalid, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cyclufenamid, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-5-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)—N-benzyl-N-([methyl(methyl-thioethylideneamino-oxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluopyram, fluoxastrobin, fluoroimide, fluquinconazole, flusilazole, flutolanil, flutriafol, fluxapyroxad, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, isopyrazam, kasugamycin, kresoxim-methyl, LY186054, LY211795, LY248908, mancozeb, mandipropamid, maneb, mefenoxam, metalaxyl, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, penflufen, penthiopyrad, phenazin oxide, phosetyl-A1, phosphorus acids, phthalide, picoxystrobin (ZA1963), polyoxinD, polyram, probenazole, prochloraz, procymidone, propamocarb, propiconazole, propineb, propionic acid, prothioconazole, pyrazophos, pyrifenox, pyrimethanil, pyraclostrobin, pyroquilon, pyroxyfur, pyrroInitrin, quaternary ammonium compounds, quinomethionate, quinoxyfen, quintozene, sedaxane, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram, N-[9-(dichloromethylene)-1,2,3,4-tetrahydro-1,4-methanonaphthalen-5-yl]-3-(difluoromethyl)-1-methyl-1H-pyrazole-4-carboxamide [1072957-71-1], 1-methyl-3-difluoromethyl-1H-pyrazole-4-carboxylic acid (2-dichloromethylene-3-ethyl-1-methyl-indan-4-yl)-amide, and 1-methyl-3-difluoromethyl-4H-pyrazole-4-carboxylic acid [2-(2,4-dichloro-phenyl)-2-methoxy-1-methyl-ethyl]-amide.

In addition, biological agents may be included in the composition of the invention e.g. Bacillus species such as Bacillus firmus, Bacillus cereus, Bacillus subtilis, and Pasteuria species such as Pasteuria penetrans and Pasteuria nishizawae. A suitable Bacillus firmus strain is strain CNCM I-1582 which is commercially available as BioNem™. A suitable Bacillus cereus strain is strain CNCM I-1562. Of both Bacillus strains more details can be found in U.S. Pat. No. 6,406,690. Other biological organisms that may be included in the compositions of the invention are bacteria such as Streptomyces spp. such as S. avermitilis, and fungi such as Pochonia spp. such as P. chlamydosporia. Also of interest are Metarhizium spp. such as M. anisopliae; Pochonia spp. such as P. chlamydosporia.

The compounds of formula (IA) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The compounds of the invention are also useful in the field of animal health, e.g. they may be used against parasitic invertebrate pests, more preferably against parasitic invertebrate pests in or on an animal. Examples of pests include nematodes, trematodes, cestodes, flies, mites, tricks, lice, fleas, true bugs and maggots. The animal may be a non-human animal, e.g. an animal associated with agriculture, e.g. a cow, a pig, a sheep, a goat, a horse, or a donkey, or a companion animal, e.g. a dog or a cat.

In a further aspect the invention provides a compound of the invention for use in a method of therapeutic treatment.

In a further aspect the invention relates to a method of controlling parasitic invertebrate pests in or on an animal comprising administering a pesticidally effective amount of a compound of the invention. The administration may be for example oral administration, parenteral administration or external administration, e.g. to the surface of the animal body. In a further aspect the invention relates to a compound of the invention for controlling parasitic invertebrate pests in or on an animal. In a further aspect the invention relates to use of a compound of the invention in the manufacture of a medicament for controlling parasitic invertebrate pests in or on an animal

In a further aspect, the invention relates to a method of controlling parasitic invertebrate pests comprising administering a pesticidally effective amount of a compound of the invention to the environment in which an animal resides.

In a further aspect the invention relates to a method of protecting an animal from a parasitic invertebrate pest comprising administering to the animal a pesticidally effective amount of a compound of the invention. In a further aspect the invention relates to a compound of the invention for use in protecting an animal from a parasitic invertebrate pest. In a further aspect the invention relates to use of a compound of the invention in the manufacture of a medicament for protecting an animal from a parasitic invertebrate pest.

In a further aspect the invention provides a method of treating an animal suffering from a parasitic invertebrate pest comprising administering to the animal a pesticidally effective amount of a compound of the invention. In a further aspect the invention relates to a compound of the invention for use in treating an animal suffering from a parasitic invertebrate pest. In a further aspect the invention relates to use of a compound of the invention in the manufacture of a medicament for treating an animal suffering from a parasitic invertebrate pest.

In a further aspect, the invention provides a pharmaceutical composition comprising a compound of the invention and a pharmaceutically suitable excipient.

The compounds of the invention may be used alone or in combination with one or more other biologically active ingredients.

In one aspect the invention provides a combination product comprising a pesticidally effective amount of a component A and a pesticidally effective amount of component B wherein component A is a compound of the invention and component B is a compound as described below.

The compounds of the invention may be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. No. 5,478,855, U.S. Pat. No. 4,639,771 and DE-19520936.

The compounds of the invention may be used in combination with derivatives and analogues of the general class of dioxomorpholine antiparasitic agents as described in WO-9615121 and also with anthelmintic active cyclic depsipeptides such as those described in WO-9611945, WO-9319053, WO-9325543, EP-626375, EP-382173, WO-9419334, EP-382173, and EP-503538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in International Patent Application Publication Numbers WO95/19363 or WO04/72086, particularly the compounds disclosed therein.

Other examples of such biologically active compounds that the compounds of the invention may be used in combination with include but are not restricted to the following:

Organophosphates: acephate, azamethiphos, azinphos-ethyl, azinphos- methyl, bromophos, bromophos-ethyl, cadusafos, chlorethoxyphos, chlorpyrifos, chlorfenvinphos, chlormephos, demeton, demeton-5-methyl, demeton-5-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl- parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos- methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, Buprofezine pyrimidifen, NC-1111, R-195,RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, Y¹-5301.

Fungicides: acibenzolar, aldimorph, ampropylfos, andoprim, azaconazole, azoxystrobin, benalaxyl, benomyl, bialaphos, blasticidin-S, Bordeaux mixture, bromuconazole, bupirimate, carpropamid, captafol, captan, carbendazim, chlorfenazole, chloroneb, chloropicrin, chlorothalonil, chlozolinate, copper oxychloride, copper salts, cyflufenamid, cymoxanil, cyproconazole, cyprodinil, cyprofuram, RH-7281, diclocymet, diclobutrazole, diclomezine, dicloran, difenoconazole, RP-407213, dimethomorph, domoxystrobin, diniconazole, diniconazole-M, dodine, edifenphos, epoxiconazole, famoxadone, fenamidone, fenarimol, fenbuconazole, fencaramid, fenpiclonil, fenpropidin, fenpropimorph, fentin acetate, fluazinam, fludioxonil, flumetover, flumorf/flumorlin, fentin hydroxide, fluoxastrobin, fluquinconazole, flusilazole, flutolanil, flutriafol, folpet, fosetyl- aluminium, furalaxyl, furametapyr, hexaconazole, ipconazole, iprobenfos, iprodione, isoprothiolane, kasugamycin, krsoxim-methyl, mancozeb, maneb, mefenoxam, mepronil, metalaxyl, metconazole, metominostrobin/fenominostrobin, metrafenone, myclobutanil, neo-asozin, nicobifen, orysastrobin, oxadixyl, penconazole, pencycuron, probenazole, prochloraz, propamocarb, propioconazole, proquinazid, prothioconazole, pyrifenox, pyraclostrobin, pyrimethanil, pyroquilon, quinoxyfen, spiroxamine, sulfur, tebuconazole, tetrconazole, thiabendazole, thifluzamide, thiophanate-methyl, thiram, tiadinil, triadimefon, triadimenol, tricyclazole, trifloxystrobin, triticonazole, validamycin, vinclozin.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

When used in combination with other active ingredients, the compounds of the invention are preferably used in combination with the following: imidacloprid, enrofloxacin, praziquantel, pyrantel embonate, febantel, penethamate, moloxicam, cefalexin, kanamycin, pimobendan, clenbuterol, fipronil, ivermectin, omeprazole, tiamulin, benazepril, milbemycin, cyromazine, thiamethoxam, pyriprole, deltamethrin, cefquinome, florfenicol, buserelin, cefovecin, tulathromycin, ceftiour, selamectin, carprofen, metaflumizone, moxidectin, methoprene (including S-methoprene), clorsulon, pyrantel, amitraz, triclabendazole, avermectin, abamectin, emamectin, eprinomectin, doramectin selamectin, nemadectin, albendazole, cambendazole, fenbendazole, flubendazole, mebendazole, oxfendazole, oxibendazole, parbendazole, tetramisole, levamisole, pyrantel pamoate, oxantel, morantel, triclabendazole, epsiprantel, fipronil, lufenuron, ecdysone or tebufenozide; more preferably, enrofloxacin, praziquantel, pyrantel embonate, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, selamectin, carprofen, moxidectin, clorsulon, pyrantel, eprinomectin, doramectin, selamectin, nemadectin, albendazole, cambendazole, fenbendazole, flubendazole, mebendazole, oxfendazole, oxibendazole, parbendazole, tetramisole, levamisole, pyrantel pamoate, oxantel, morantel, triclabendazole, epsiprantel, lufenuron or ecdysone; even more preferably enrofloxacin, praziquantel, pyrantel embonate, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, selamectin, carprofen, moxidectin, clorsulon or pyrantel. Examples of mixing partner ratios for all mixtures described herein include 100:1 to 1:6000, 50:1 to 1:50, 20:1 to 1:20, even more especially from 10:1 to 1:10, 5:1 to 1:5, 2:1 to 1:2, 4:1 to 2:1, 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are understood to include, on the one hand, ratios by weight and also, on other hand, molar ratios.

Of particular note is a combination where the additional active ingredient has a different site of action from the compound of formula I. In certain instances, a combination with at least one other parasitic invertebrate pest control active ingredient having a similar spectrum of control but a different site of action will be particularly advantageous for resistance management. Thus, a combination product of the invention may comprise a pesticidally effective amount of a compound of formula (IA) and pesticidally effective amount of at least one additional parasitic invertebrate pest control active ingredient having a similar spectrum of control but a different site of action.

One skilled in the art recognizes that because in the environment and under physiological conditions salts of chemical compounds are in equilibrium with their corresponding non salt forms, salts share the biological utility of the non salt forms.

Thus a wide variety of salts of compounds of the invention (and active ingredients used in combination with the active ingredients of the invention) may be useful for control of invertebrate pests and animal parasites. Salts include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

The compounds of the invention also include N-oxides. Accordingly, the invention comprises combinations of compounds of the invention including N-oxides and salts thereof and an additional active ingredient including N-oxides and salts thereof.

The compositions for use in animal health may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compounds of the invention can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of the combination products. Compositions with spray oils, spray oil concentrations, spreader stickers, adjuvants, other solvents, and synergists such as piperonyl butoxide often enhance compound efficacy. Such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as the case may be. Of note is a spray composition comprising a pesticidally effective amount of a compound of the invention and a carrier. One embodiment of such a spray composition comprises a pesticidally effective amount of a compound of the invention and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one parasitic invertebrate pest selected from the group consisting of mosquitoes, black flies, stable flies, deer flies, horse flies, wasps, yellow jackets, hornets, ticks, spiders, ants, gnats, and the like, including individually or in combinations.

The controlling of animal parasites includes controlling external parasites that are parasitic to the surface of the body of the host animal (e.g., shoulders, armpits, abdomen, inner part of the thighs) and internal parasites that are parasitic to the inside of the body of the host animal (e.g., stomach, intestine, lung, veins, under the skin, lymphatic tissue). External parasitic or disease transmitting pests include, for example, chiggers, ticks, lice, mosquitoes, flies, mites and fleas. Internal parasites include heartworms, hookworms and helminths. The compounds of the invention may be particularly suitable for combating external parasitic pests. The compounds of the invention may be suitable for systemic and/or non-systemic control of infestation or infection by parasites on animals.

The compounds of the invention may be suitable for combating parasitic invertebrate pests that infest animal subjects including those in the wild, livestock and agricultural working animals. Livestock is the term used to refer (singularly or plurally) to a domesticated animal intentionally reared in an agricultural setting to make produce such as food or fiber, or for its labor; examples of livestock include cattle, sheep, goats, horses, pigs, donkeys, camels, buffalo, rabbits, hens, turkeys, ducks and geese (e.g., raised for meat, milk, butter, eggs, fur, leather, feathers and/or wool), cultured fish, honeybees. By combating parasites, fatalities and performance reduction (in terms of meat, milk, wool, skins, eggs, etc.) are reduced, so that applying the compounds of the invention allows more economic and simple husbandry of animals.

By controlling these pests it is intended to reduce deaths and improve performance (in the case of meat, milk, wool, hides, eggs, honey and the like) and health of the host animal. Also, controlling parasites may help to prevent the transmittance of infectious agents, the term “controlling” referring to the veterinary field, meaning that the active compounds are effective in reducing the incidence of the respective parasite in an animal infected with such parasites to innocuous levels, e.g. the active compound is effective in killing the respective parasite, inhibiting its growth, or inhibiting its proliferation.

The compounds of the invention may be suitable for combating parasitic invertebrate pests that infest companion animals and pets (e.g., dogs, cats, pet birds and aquarium fish), research and experimental animals (e.g., hamsters, guinea pigs, rats and mice), as well as animals raised for/in zoos, wild habitats and/or circuses.

In an embodiment of this invention, the animal is preferably a vertebrate, and more preferably a mammal, avian or fish. In a particular embodiment, the animal subject is a mammal (including great apes, such as humans). Other mammalian subjects include primates (e.g., monkeys), bovine (e.g., cattle or dairy cows), porcine (e.g., hogs or pigs), ovine (e.g., goats or sheep), equine (e.g., horses), canine (e.g., dogs), feline (e.g., house cats), camels, deer, donkeys, buffalos, antelopes, rabbits, and rodents (e.g., guinea pigs, squirrels, rats, mice, gerbils, and hamsters). Avians include Anatidae (swans, ducks and geese), Columbidae (e.g., doves and pigeons), Phasianidae (e.g., partridges, grouse and turkeys), Thesienidae (e.g., domestic chickens), Psittacines (e.g., parakeets, macaws, and parrots), game birds, and ratites (e.g., ostriches).

Birds treated or protected by the compounds of the invention can be associated with either commercial or noncommercial aviculture. These include Anatidae, such as swans, geese, and ducks, Columbidae, such as doves and domestic pigeons, Phasianidae, such as partridge, grouse and turkeys, Thesienidae, such as domestic chickens, and Psittacines, such as parakeets, macaws and parrots raised for the pet or collector market, among others.

For purposes of the present invention, the term “fish” is understood to include without limitation, the Teleosti grouping of fish, i.e., teleosts. Both the Salmoniformes order (which includes the Salmonidae family) and the Perciformes order (which includes the Centrarchidae family) are contained within the Teleosti grouping. Examples of potential fish recipients include the Salmonidae, Serranidae, Sparidae, Cichlidae, and Centrarchidae, among others.

Other animals are also contemplated to benefit from the inventive methods, including marsupials (such as kangaroos), reptiles (such as farmed turtles), and other economically important domestic animals for which the inventive methods are safe and effective in treating or preventing parasite infection or infestation.

Examples of parasitic invertebrate pests controlled by administering a pesticidally effective amount of the compounds of the invention to an animal to be protected include ectoparasites (arthropods, acarines, etc.) and endoparasites (helminths, e.g., nematodes, trematodes, cestodes, acanthocephalans, etc. and protozoae, such as coccidia).

The disease or group of diseases described generally as helminthiasis is due to infection of an animal host with parasitic worms known as helminths. The term ‘helminths’ is meant to include nematodes, trematodes, cestodes and acanthocephalans. Helminthiasis is a prevalent and serious economic problem with domesticated animals such as swine, sheep, horses, cattle, goats, dogs, cats and poultry.

Among the helminths, the group of worms described as nematodes causes widespread and at times serious infection in various species of animals.

Nematodes that are contemplated to be treated by the compounds of the invention include, without limitation, the following genera: Acanthocheilonema, Aelurostrongylus, Ancylostoma, Angiostrongylus, Ascaridia, Ascaris, Brugia, Bunostomum, Capillaria, Chabertia, Cooperia, Crenosoma, Dictyocaulus, Dioctophyme, Dipetalonema, Diphyllobothrium, Dirofilaria, Dracunculus, Enterobius, Filaroides, Haemonchus, Heterakis, Lagochilascaris, Loa, Mansonella, Muellerius, Necator, Nematodirus, Oesophagostomum, Ostertagia, Oxyuris, Parafilaria, Parascaris, Physaloptera, Protostrongylus, Setaria, Spirocerca, Stephanofilaria, Strongyloides, Strongylus, Thelazia, Toxascaris, Toxocara, Trichinella, Trichonema, Trichostrongylus, Trichuris, Uncinaria and Wuchereria.

Of the above, the most common genera of nematodes infecting the animals referred to above are Haemonchus, Trichostrongylus, Ostertagia, Nematodirus, Cooperia, Ascaris, Bunostomum, Oesophagostomum, Chabertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. Certain of these, such as Nematodirus, Cooperia and Oesophagostomum attack primarily the intestinal tract while others, such as Haemonchus and Ostertagia, are more prevalent in the stomach while others such as Dictyocaulus are found in the lungs. Still other parasites may be located in other tissues such as the heart and blood vessels, subcutaneous and lymphatic tissue and the like.

Trematodes that are contemplated to be treated by the invention and by the inventive methods include, without limitation, the following genera: Alaria, Fasciola, Nanophyetus, Opisthorchis, Paragonimus and Schistosoma.

Cestodes that are contemplated to be treated by the invention and by the inventive methods include, without limitation, the following genera: Diphyllobothrium, Diplydium, Spirometra and Taenia.

The most common genera of parasites of the gastrointestinal tract of humans are Ancylostoma, Necator, Ascaris, Strongy hides, Trichinella, Capillaria, Trichuris and Enterobius. Other medically important genera of parasites which are found in the blood or other tissues and organs outside the gastrointestinal tract are the filarial worms such as Wuchereria, Brugia, Onchocerca and Loa, as well as Dracunculus and extra intestinal stages of the intestinal worms Strongyloides and Trichinella.

Numerous other helminth genera and species are known to the art, and are also contemplated to be treated by the compounds of the invention. These are enumerated in great detail in Textbook of Veterinary Clinical Parasitology, Volume 1, Helminths, E. J. L. Soulsby, F. A. Davis Co., Philadelphia, Pa.; Helminths, Arthropods and Protozoa, (6^{th}Edition of Monnig's Veterinary Helminthology and Entomology), E. J. L. Soulsby, Williams and Wilkins Co., Baltimore, Md.

The compounds of the invention may be effective against a number of animal ectoparasites (e.g., arthropod ectoparasites of mammals and birds in particular insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like).

Insect and acarine pests include, e.g., biting insects such as flies and mosquitoes, mites, ticks, lice, fleas, true bugs, parasitic maggots, and the like.

Adult flies include, e.g., the horn fly or Haematobia irritans, the horse fly or Tabanus spp., the stable fly or Stomoxys calcitrans, the black fly or Simulium spp., the deer fly or Chrysops spp., the louse fly or Melophagus ovinus, and the tsetse fly or Glossina spp. Parasitic fly maggots include, e.g., the bot fly (Oestrus ovis and Cuterebra spp.), the blow fly or Phaenicia spp., the screwworm or Cochliomyia hominivorax, the cattle grub or Hypoderma spp., the fleeceworm and the Gastrophilus of horses. Mosquitoes include, for example, Culex spp., Anopheles spp. and Aedes spp.

Mites include Mesostigmalphatalpha spp. e.g., mesostigmatids such as the chicken mite, Dermalphanyssus galphallinalphae; itch or scab mites such as Sarcoptidae spp. for example, Salpharcoptes scalphabiei; mange mites such as Psoroptidae spp. including Chorioptes bovis and Psoroptes ovis; chiggers e.g., Trombiculidae spp. for example the North American chigger, Trombiculalpha alphalfreddugesi.

Ticks include, e.g., soft-bodied ticks including Argasidae spp. for example Argalphas spp. and Ornithodoros spp.; hard-bodied ticks including Ixodidae spp., for example Rhipicephalphalus sanguineus, Dermacentor variabilis, Dermacentor andersoni, Amblyomma americanum, Ixodes scapularis and other Rhipicephalus spp. (including the former Boophilus genera).

Lice include, e.g., sucking lice, e.g., Menopon spp. and Bovicola spp.; biting lice, e.g., Haematopinus spp., Linognathus spp. and Solenopotes spp.

Fleas include, e.g., Ctenocephalides spp., such as dog flea (Ctenocephalides canis) and cat flea (Ctenocephalides felis); Xenopsylla spp. such as oriental rat flea (Xenopsylla cheopis); and Pulex spp. such as human flea (Pulex irritans).

True bugs include, e.g., Cimicidae or e.g., the common bed bug (Cimex lectularius); Triatominae spp. including triatomid bugs also known as kissing bugs; for example Rhodnius prolixus and Triatoma spp.

Generally, flies, fleas, lice, mosquitoes, gnats, mites, ticks and helminths cause tremendous losses to the livestock and companion animal sectors. Arthropod parasites also are a nuisance to humans and can vector disease-causing organisms in humans and animals.

Numerous other parasitic invertebrate pests are known to the art, and are also contemplated to be treated by the compounds of the invention. These are enumerated in great detail in Medical and Veterinary Entomology, D. S. Kettle, John Wiley AND Sons, New York and Toronto; Control of Arthropod Pests of Livestock: A Review of Technology, R. O. Drummand, J. E. George, and S. E. Kunz, CRC Press, Boca Raton, Fla.

The compounds of the invention may also be effective against ectoparasites, e.g. insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like. These include e.g. flies such as Haematobia (Lyperosia) irritans (horn fly), Simulium spp. (blackfly), Glossina spp. (tsetse flies), Hydrotaea irritans (head fly), Musca autumnalis (face fly), Musca domestica (house fly), Morellia simplex (sweat fly), Tabanus spp. (horse fly), Hypoderma bovis, Hypoderma lineatum, Lucilia sericata, Lucilia cuprina (green blowfly), Calliphora spp. (blowfly), Protophormia spp., Oestrus ovis (nasal botfly), Culicoides spp. (midges), Hippobosca equine, Gastrophilus intestinalis, Gastrophilus haemorrhoidalis and Gastrophilus nasalis; lice such as Bovicola (Damalinia) bovis, Bovicola equi, Haematopinus asini, Felicola subrostratus, Heterodoxus spiniger, Lignonathus setosus and Trichodectes canis; keds such as Melophagus ovinus; and mites such as Psoroptes spp., Sarcoptes scabei, Chorioptes bovis, Demodex equi, Cheyletiella spp., Notoedres cati, Trombicula spp. and Otodectes cyanotis (ear mites).

Examples of species of animal health pesets include those from the order of the Anoplurida, for example Haematopinus spp., Linognathus spp., Pediculus spp., Phtirus spp., Solenopotes spp.; particular examples are: Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus; from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; particular examples are: Bovicola bovis, Bovicola ovis, Bovicola limbata, Damalina bovis, Trichodectes canis, Felicola subrostratus, Bovicola caprae, Lepikentron ovis, Werneckiella equi; from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; particular examples are: Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus inermis, Gasterophilus nasalis, Gasterophilus nigricornis, Gasterophilus pecorum, Braula coeca; from the order of the Siphonapterida, for example Pulex spp., Ctenocephalides spp., Tunga spp., Xenopsylla spp., Ceratophyllus spp.; particular examples are: Ctenocephalides canis, Ctenocephalides felis, Pulex irritans, Tunga penetrans, Xenopsylla cheopis; from the order of the Heteropterida, for example Cimex spp., Triatoma spp., Rhodnius spp., Panstrongylus spp; from the order of the Blattarida, for example Blatta orientalis, Periplaneta americana, Blattela germanica, Supella spp. (e.g. Suppella longipalpa); from the subclass of the Acari (Acarina) and the orders of the Meta- and Mesostigmata, for example Argas spp., Ornithodorus spp., Otobius spp., Ixodes spp., Amblyomma spp., Rhipicephalus (Boophilus) spp Dermacentor spp., Haemophysalis spp., Hyalomma spp., Dermanyssus spp., Rhipicephalus spp. (the original genus of multi host ticks) Ornithonyssus spp., Pneumonyssus spp., Raillietia spp., Pneumonyssus spp., Sternostoma spp., Varroa spp., Acarapis spp.; particular examples are: Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsoni; from the order of the Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particular examples are: Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleri, Neoschongastia xerothermobia, Trombicula akamushi, Otodectes cynotis, Notoedres cati, Sarcoptis canis, Sarcoptes bovis, Sarcoptes ovis, Sarcoptes rupicaprae (S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi; Gasterophilus spp., Stomoxys spp., Trichodectes spp., Rhodnius spp., Ctenocephalides canis, Cimx lecturius, Ctenocephalides felis, Lucilia cuprina; examples of acari include Ornithodoros spp., Ixodes spp., Boophilus spp.

Treatments of the invention are by conventional means such as by enteral administration in the form of, for example, tablets, capsules, drinks, drenching preparations, granulates, pastes, boli, feed-through procedures, or suppositories; or by parenteral administration, such as, for example, by injection (including intramuscular, subcutaneous, intravenous, intraperitoneal) or implants; or by nasal administration; or by dermal application in the form of, for example, bathing or dipping, spraying, pouring-on and spotting-on, washing, dusting, and with the aid of active-compound-comprising shaped articles such as collars, ear tags, tail tags, limb bands, halters, marking devices and the like.

When compounds of the invention are applied in combination with an additional biologically active ingredient, they may be administered separately e.g. as separate compositions. In this case, the biologically active ingredients may be administered simultaneously or sequentially. Alternatively, the biologically active ingredients may be components of one composition.

The compounds of the invention may be administered in a controlled release form, for example in subcutaneous or orally adminstered slow release formulations.

Typically a parasiticidal composition according to the present invention comprises a compound of the invention, optionally in combination with an additional biologically active ingredient, or N-oxides or salts thereof, with one or more pharmaceutically or veterinarily acceptable carriers comprising excipients and auxiliaries selected with regard to the intended route of administration (e.g., oral or parenteral administration such as injection) and in accordance with standard practice. In addition, a suitable carrier is selected on the basis of compatibility with the one or more active ingredients in the composition, including such considerations as stability relative to pH and moisture content. Therefore of note are compounds of the invention for protecting an animal from an invertebrate parasitic pest comprising a parasitically effective amount of a compound of the invention, optionally in combination with an additional biologically active ingredient and at least one carrier.

For parenteral administration including intravenous, intramuscular and subcutaneous injection, the compounds of the invention can be formulated in suspension, solution or emulsion in oily or aqueous vehicles, and may contain adjuncts such as suspending, stabilizing and/or dispersing agents.

The compounds of the invention may also be formulated for bolus injection or continuous infusion. Pharmaceutical compositions for injection include aqueous solutions of water-soluble forms of active ingredients (e.g., a salt of an active compound), preferably in physiologically compatible buffers containing other excipients or auxiliaries as are known in the art of pharmaceutical formulation. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes.

Aqueous injection suspensions may contain substances that increase the viscosity of the suspension, such as sodium carboxymethyl cellulose, sorbitol, or dextran. Formulations for injection may be presented in unit dosage form, e.g., in ampoules or in multi-dose containers. Alternatively, the active ingredient may be in powder form for constitution with a suitable vehicle, e.g., sterile, pyrogen-free water, before use.

In addition to the formulations described supra, the compounds of the invention may also be formulated as a depot preparation. Such long acting formulations may be administered by implantation (for example, subcutaneously or intramuscularly) or by intramuscular or subcutaneous injection.

The compounds of the invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

For administration by inhalation, the compounds of the invention can be delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds of the invention may have favourable pharmacokinetic and pharmacodynamic properties providing systemic availability from oral administration and ingestion. Therefore after ingestion by the animal to be protected, parasiticidally effective concentrations of a compound of the invention in the bloodstream may protect the treated animal from blood-sucking pests such as fleas, ticks and lice. Therefore of note is a composition for protecting an animal from an invertebrate parasite pest in a form for oral administration (i.e. comprising, in addition to a parasiticidally effective amount of a compound of the invention, one or more carriers selected from binders and fillers suitable for oral administration and feed concentrate carriers).

For oral administration in the form of solutions (the most readily available form for absorption), emulsions, suspensions, pastes, gels, capsules, tablets, boluses, powders, granules, rumen-retention and feed/water/lick blocks, the compounds of the invention can be formulated with binders/fillers known in the art to be suitable for oral administration compositions, such as sugars and sugar derivatives (e.g., lactose, sucrose, mannitol, sorbitol), starch (e.g., maize starch, wheat starch, rice starch, potato starch), cellulose and derivatives (e.g., methylcellulose, carboxymethylcellulose, ethylhydroxycellulose), protein derivatives (e.g., zein, gelatin), and synthetic polymers (e.g., polyvinyl alcohol, polyvinylpyrrolidone). If desired, lubricants (e.g., magnesium stearate), disintegrating agents (e.g., cross-linked polyvinylpyrrolidinone, agar, alginic acid) and dyes or pigments can be added. Pastes and gels often also contain adhesives (e.g., acacia, alginic acid, bentonite, cellulose, xanthan gum, colloidal magnesium aluminum silicate) to aid in keeping the composition in contact with the oral cavity and not being easily ejected.

In one embodiment a composition of the present invention is formulated into a chewable and/or edible product (e.g., a chewable treat or edible tablet). Such a product would ideally have a taste, texture and/or aroma favored by the animal to be protected so as to facilitate oral administration of the compounds of the invention.

If the parasiticidal compositions are in the form of feed concentrates, the carrier is typically selected from high-performance feed, feed cereals or protein concentrates.

Such feed concentrate-containing compositions can, in addition to the parasiticidal active ingredients, comprise additives promoting animal health or growth, improving quality of meat from animals for slaughter or otherwise useful to animal husbandry.
These additives can include, for example, vitamins, antibiotics, chemotherapeutics, bacteriostats, fungistats, coccidiostats and hormones.

The compound of the invention may also be formulated in rectal compositions such as suppositories or retention enemas, using, e.g., conventional suppository bases such as cocoa butter or other glycerides.

The formulations for the method of this invention may include an antioxidant, such asBHT (butylated hydroxytoluene). The antioxidant is generally present in amounts of at 0.1-5 percent (wt/vol). Some of the formulations require a solubilizer, such as oleic acid, to dissolve the active agent, particularly if spinosad is included. Common spreading agents used in these pour-on formulations include isopropyl myristate, isopropyl palmitate, caprylic/capric acid esters of saturated C₁₂-C₁₈ fatty alcohols, oleic acid, oleyl ester, ethyl oleate, triglycerides, silicone oils and dipropylene glycol methyl ether. The pour-on formulations for the method of this invention are prepared according to known techniques. Where the pour-on is a solution, the parasiticide/insecticide is mixed with the carrier or vehicle, using heat and stirring if required. Auxiliary or additional ingredients can be added to the mixture of active agent and carrier, or they can be mixed with the active agent prior to the addition of the carrier. Pour-on formulations in the form of emulsions or suspensions are similarly prepared using known techniques.

Other delivery systems for relatively hydrophobic pharmaceutical compounds may be employed. Liposomes and emulsions are well-known examples of delivery vehicles or carriers for hydrophobic drugs. In addition, organic solvents such as dimethylsulfoxide may be used, if needed.

The rate of application required for effective parasitic invertebrate pest control (e.g. “pesticidally effective amount”) will depend on such factors as the species of parasitic invertebrate pest to be controlled, the pest's life cycle, life stage, its size, location, time of year, host crop or animal, feeding behavior, mating behavior, ambient moisture, temperature, and the like. One skilled in the art can easily determine the pesticidally effective amount necessary for the desired level of parasitic invertebrate pest control.

In general for veterinary use, the compounds of the invention are administered in a pesticidally effective amount to an animal, particularly a homeothermic animal, to be protected from parasitic invertebrate pests.

A pesticidally effective amount is the amount of active ingredient needed to achieve an observable effect diminishing the occurrence or activity of the target parasitic invertebrate pest. One skilled in the art will appreciate that the pesticidally effective dose can vary for the various compounds and compositions useful for the method of the present invention, the desired pesticidal effect and duration, the target parasitic invertebrate pest species, the animal to be protected, the mode of application and the like, and the amount needed to achieve a particular result can be determined through simple experimentation.

For oral or parenteral administration to animals, a dose of the compositions of the present invention administered at suitable intervals typically ranges from about 0.01 mg/kg to about 100 mg/kg, and preferably from about 0.01 mg/kg to about 30 mg/kg of animal body weight.

Suitable intervals for the administration of the compositions of the present invention to animals range from about daily to about yearly. Of note are administration intervals ranging from about weekly to about once every 6 months. Of particular note are monthly administration intervals (i.e. administering the compounds to the animal once every month).

formula (IA) The following abbreviations were used throughout this section: s=singlet; bs=broad singlet; d=doublet; dd=double doublet; dt=double triplet; t=triplet, tt=triple triplet, q=quartet, sept=septet; m=multiplet; Me=methyl; Et=ethyl; Pr=propyl; Bu=butyl; RT=retention time; MH⁺=molecular cation.

### PREPARATION EXAMPLES

The following preparation examples describe synthesis of compounds of formula (IA) and intermediates thereof.

### Example P1

### 4-(3,5-Dichloro-phenyl)-5,5,5-trifluoro-pent-2-yne-1,4-diol

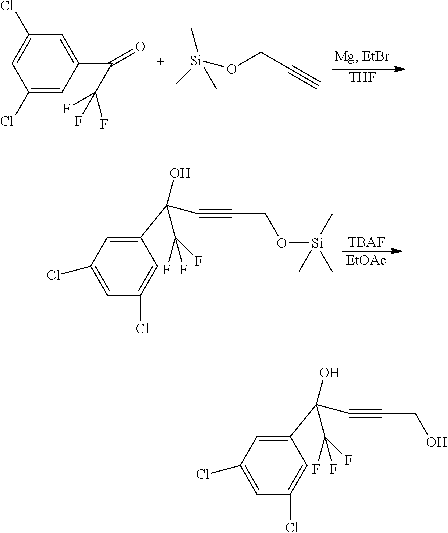

To a stirring solution of magnesium (520 mg) in anhydrous tetrahydrofuran (50 mL) under argon at room temperature, was added ethyl bromide (1.7 mL). After stirring for 2 hours at room temperature, the solution was cooled to 0° C. and Trimethyl-prop-2-ynyloxy-silane (3.1 mL) was added. The solution was allowed to warm to room temperature and then after 40 minutes, it was cooled again to 0° C. To this cooled solution, 1-(3,5-Dichloro-phenyl)-2,2,2-trifluoro-ethanone (5 g) (Journal of Physical Organic Chemistry (1989), 2(4), 363-6) were added. The solution was stirred at 0° C. for 1 hour. The mixture was quenched with saturated ammonium chloride and then extracted with diethyl ether. The combined organic extracts were dried over magnesium sulfate and concentrated. The residue was then dissolved in ethyl acetate (60 mL) and the solution was stirred at room temperature under argon. A solution of tetrabutylammonium fluoride (21 mL of a 1 M solution in tetrahydrofuran) was added. The solution was stirred for one hour then was allowed to stand at room temperature for 21 hours. The mixture was quenched with saturated ammonium chloride and then extracted with diethyl ether. The combined organic extracts were dried over magnesium sulfate and concentrated. The residue was purified by chromatography on silica gel (eluent: heptane/ethyl acetate 1:0 to 7:3) to give 4-(3,5-Dichloro-phenyl)-5,5,5-trifluoro-pent-2-yne-1,4-diol (3.798 g) as a colorless oil.

¹H-NMR (CDCl₃, 400 MHz): 7.61 (m, 2H), 7.43 (t, J=1.83 Hz, 1H), 4.44 (m, 2H), 3.45 (s, 1H) ppm.

### Example P2

### Tributyl-[5-(3,5-dichloro-phenyl)-5-trifluoromethyl-2,5-dihydro-furan-3-yl]-stannane

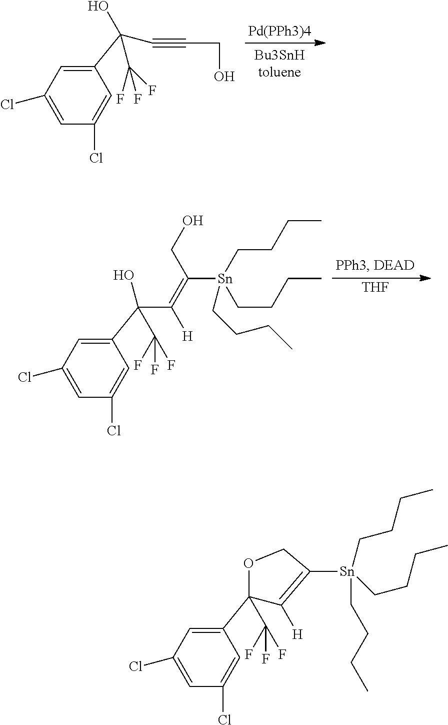 [SMILES: OCC#CC(O)(c1cc(Cl)cc(Cl)c1)C(F)(F)F ; CCC[CH2][Sn]([CH2]CCC)([CH2]CCC)/[C](=C/C(O)(c1cc(Cl)cc(Cl)c1)C(F)(F)F)CO ; CCC[CH2][Sn]([CH2]CCC)([CH2]CCC)[C]1=CC(c2cc(Cl)cc(Cl)c2)(C(F)(F)F)OC1 ; "THF" ; "Pd(PPh3)4"]

To a solution of 4-(3,5-Dichloro-phenyl)-5,5,5-trifluoro-pent-2-yne-1,4-diol (2.5 g) in toluene under argon, was added tetrakis(triphenylphosphine) palladium (190 mg) and tributyltinhydride (2.25 mL). The reaction mixture was stirred for 45 minutes then the solvent was evaporated under vacuo. The residue was then dissolved in anhydrous tetrahydrofuran (50 mL) with triphenylphosphine (2.19 g) and the solution was stirred at 0° C. under argon. To this solution was slowly added diethyl azodicarboxylate (1.31 mL). The mixture was stirred at 0° C. for 90 minutes then the solvent was evaporated under vacuo.

The residue was partitioned between acetonitrile and heptane and the acetonitrile part was washed twice with heptane. The combined heptane extracts were combined and evaporated to give a residue that was purified by chromatography on silica gel (eluent: heptane) to give Tributyl-[5-(3,5-dichloro-phenyl)-5-trifluoromethyl-2,5-dihydro-furan-3-yl]-stannane (1.587 g) as a colorless oil.

¹H-NMR (CDCl₃, 400 MHz): 7.43 (m, 2H), 7.34 (d, J=1.83 Hz, 1H), 5.93 (t, J=2.57 Hz, 1H), 5.02-4.89 (2×dd, J=2.6 and 13.6 and 1.8 Hz, 2H), 1.52-1.46 (m, 6H), 1.34-1.28 (m, 6H), 1.03-0.99 (t, J=8.1 Hz, 6H), 0.89 (t, J=7.3 Hz, 9H) ppm.

### Example P3

### Preparation of 4-bromo-2-methyl-benzoic acid tert-butyl ester

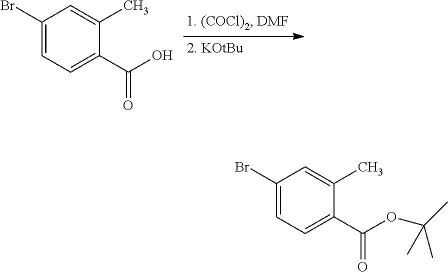

4-Bromo-2-methyl-benzoic acid (commercially available) (50 g) was suspended in dichloromethane (500 mL). A catalytic amount of N,N-dimethylformamide (“DMF”) and oxalyl chloride (23 mL) were added to the suspension. The reaction mixture was stirred at ambient temperature for 3 hours. The reaction mixture was concentrated and the residue dissolved in dry tetrahydrofuran (800 mL). The solution was cooled to 2° C. and added to a solution of potassium tert-butoxide (39.2 g) in dry tetrahydrofuran (300 mL) dropwise at 5-10° C. The reaction mixture was stirred at ambient temperature for 30 minutes and then poured onto a mixture of ice and water. The mixture was extracted with ethyl acetate. The organic extract was washed with water, dried over sodium sulfate and concentrated to give 4-bromo-2-methyl-benzoic acid tert-butyl ester (65.3 g) as yellow oil.

¹H-NMR (CDCl₃, 400 MHz): 7.70 (d, 1H), 7.40 (s, 1H), 7.35 (d, 1H), 2.58 (s, 3H), 1.60 (s, 9H).

### Example P4

### 4-[5-(3,5-Dichloro-phenyl)-5-trifluoromethyl-2,5-dihydro-furan-3-yl]-2-methyl-benzoic acid tert-butyl ester

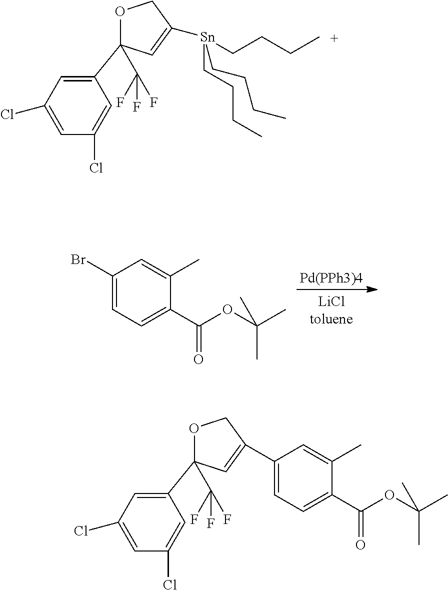

To a solution of Tributyl-[5-(3,5-dichloro-phenyl)-5-trifluoromethyl-2,5-dihydro-furan-3-yl]-stannane (1.587 g) in toluene (15 mL) under argon was successively added 4-Bromo-2-methyl-benzoic acid tert-butyl ester (630 mg), lithium chloride (600 mg) and then tetrakis(triphenylphosphine) palladium (110 mg). The reaction was refluxed at 100° C. under argon for 3 hours 30 minutes. The reaction was allowed to cool down to room temperature then after 3 hours, more tetrakis(triphenylphosphine) palladium (45 mg) was added. The solution was refluxed for a further 1 h45 and then the reaction was stopped. The mixture was cooled to room temperature and then the solvent was evaporated in vacuo to give a residue which was purified by chromatography on silica gel (eluent: heptanes\diethyl ether, from 1:0 to 9:1) to give 4-[5-(3,5-Dichloro-phenyl)-5-trifluoromethyl-2,5-dihydro-furan-3-yl]-2-methyl-benzoic acid tert-butyl ester (704 mg) as a white solid. Recrystallisation in heptane/ethyl acetate provided white crystals, m.p=160-162° C.

¹H-NMR (CDCl₃, 400 MHz): 7.83 (d, J=8.4 Hz, 1H), 7.50 (m, 2H), 7.38 (t, J=1.4 Hz, 1H), 7.22 (m, 2H), δ 6.39 (m, 1H), 5.32 (dd, J=2.2 and 12.5 Hz, 1H), 5.20 (m, 1H), 2.59 (s, 3H), 1.61 (s, 9H) ppm.

### Example P5

### 4-[5-(3,5-Dichloro-phenyl)-5-trifluoromethyl-2,5-dihydro-furan-3-yl]-2-methyl-benzoic acid

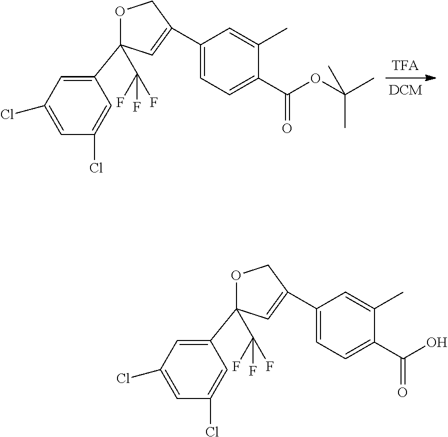 [SMILES: Cc1cc(C2=CC(c3cc(Cl)cc(Cl)c3)(C(F)(F)F)OC2)ccc1C(=O)OC(C)(C)C ; Cc1cc(C2=CC(c3cc(Cl)cc(Cl)c3)(C(F)(F)F)OC2)ccc1C(=O)O]

To a solution of 4-[5-(3,5-Dichloro-phenyl)-5-trifluoromethyl-2,5-dihydro-furan-3-yl]-2-methyl-benzoic acid tert-butyl ester (322 mg) in dichloromethane (8 mL) was added trifluoroacetic acid (0.5 mL). The reaction mixture was stirred at room temperature for 3 h30 then the solution was concentrated under vacuo to give 4-[5-(3,5-Dichloro-phenyl)-5-trifluoromethyl-2,5-dihydro-furan-3-yl]-2-methyl-benzoic acid (200 mg) as a white foam.

¹H-NMR (CDCl₃, 400 MHz): 8.07 (d, J=8.8 Hz, 1H), 7.49 (m, 2H), 7.39 (t, J=1.4 Hz, 1H), 7.3-7.26 (m, 2H), 6.46 (m, 1H), 5.34 (dd, J=2.2 and 12.5 Hz, 1H), 5.22 (m, 1H), 2.67 (s, 3H) ppm.

### Example P6

### (Compound A1 from Table A): 4-[5-(3,5-Dichloro-phenyl)-5-trifluoromethyl-2,5-dihydro-furan-3-yl]-2-methyl-N-[(2,2,2-trifluoro-ethylcarbamoyl)-ethyl]-benzamide

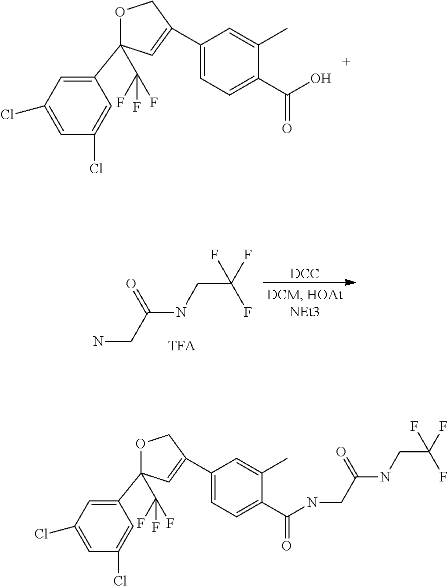

To a stirred solution of 4-[5-(3,5-Dichloro-phenyl)-5-trifluoromethyl-2,5-dihydro-furan-3-yl]-2-methyl-benzoic acid (50 mg) in dichloromethane (2 mL) was added triethylamine (0.04 mL) at ambient temperature. The solution was then stirred for 5 min under argon and the trifluoroacetate salt of 2-amino-N-(2,2,2-trifluoro-ethyl)-acetamide (39 mg, prepared according to JP2009173621) was added. To this solution, 1-hydroxyazabenzotriazole (18 mg) then N,N′-Dicyclohexylcarbodiimide (27 mg) were added. The solution was stirred for 80 minutes then was allowed to stand at ambient temperature for 3 days. The solution was concentrated under vacuo and then the crude residue was first purified by chromatography on silica gel (eluent: heptane/ethyl acetate, from 1:0 to 6:4). The residue was further recrystallised and repurified by preparative HPLC to give 4-[5-(3,5-Dichloro-phenyl)-5-trifluoromethyl-2,5-dihydro-furan-3-yl]-2-methyl-N-[(2,2,2-trifluoro-ethylcarbamoyl)-methyl]-benzamide (30 mg) as a white solid.

¹H-NMR (CDCl₃, 400 MHz): 7.49 (m, 2H), 7.44 (d, 1H, J=7.7 Hz), 7.38 (m, 1H), 7.26-7.22 (m, 2H), 7.00 (m, 1H), 6.69 (m, 1H), 6.39 (m, 1H), 5.32 (dd, J=2.2 and 12.5 Hz, 1H), 5.19 (m, 1H), 4.22 (d, J=5.14 Hz, 2H), 4.00-3.92 (m, 2H), 2.47 (s, 3H) ppm.

Similarly, using the trifluoroacetate salt of (S)-4-Amino-2-ethyl-isoxazolidin-3-one and 1-(3-Dimethylaminopropyl)-3-ethylcarbodiimide hydrochloride and 1-hydroxyazabenzotriazole as coupling agents, 4-[5-(3,5-Dichloro-phenyl)-5-trifluoromethyl-2,5-dihydro-furan-3-yl]-N—((S)-2-ethyl-3-oxo-isoxazolidin-4-yl)-2-methyl-benzamide (compound A16 from Table A): could be prepared. ¹H-NMR (CDCl₃, 400 MHz): 7.49-7.48 (m, 3H), 7.38 (m, 1H), 7.27-7.21 (m, 2H), 6.43 (s, 1H), 6.39 (s, 1H), 5.32 (dd, J=2.2 and 12.4 Hz, 1H), 5.18 (bd, 1H, J=12.2 Hz), 5.00 (t, 1H, J=8.07 Hz), 4.88-4.82 (m, 1H), 4.08-4.03 (m, 1H), 3.75-3.62 (m, 2H), 2.49 (s, 3H), 1.27 (m, 3H) ppm. ¹⁹F-NMR (CDCl₃, 376 MHz): −78.13 ppm.

Similarly, using 2-Chloro-6-[5-(3,5-dichloro-phenyl)-5-trifluoromethyl-2,5-dihydro-furan-3-yl]-nicotinic acid as a starting material and 1-(3-Dimethylaminopropyl)-3-ethylcarbodiimide hydrochloride and 1-hydroxyazabenzotriazole as coupling agents, 2-Chloro-6-[5-(3,5-dichloro-phenyl)-5-trifluoromethyl-2,5-dihydro-furan-3-yl]-N-[(2,2,2-trifluoro- ethylcarbamoyl)-methyl]-nicotinamide (compound F1 from Table F) could be prepared. ¹H-NMR (CDCl₃, 400 MHz): 8.16 (d, 1H, J=7.70 Hz), 7.49 (m, 2H), 7.46-7.43 (m, 2H), 7.39 (t, 1H, J=1.83 Hz), 6.81 (m, 1H), 6.69 (m, 1H), 5.40 (dd, J=2.2 and 13.2 Hz, 1H), 5.27 (bd, 1H, J=13.2 Hz), 4.26 (d, 2H, J=5.14 Hz), 4.02-3.94 (m, 2H) ppm. ¹⁹F-NMR (CDCl₃, 376 MHz): −77.88 and −72.44 ppm.

Similarly, using 2-Chloro-6-[5-(3,5-dichloro-phenyl)-5-trifluoromethyl-2,5-dihydro-furan-3-yl]-nicotinic acid as a starting material, 1-(3-Dimethylaminopropyl)-3-ethylcarbodiimide hydrochloride and 1-hydroxyazabenzotriazole as coupling agents, and the trifluoroacetate salt of 1,1-Dioxo-1lambda*6*-thietan-3-ylamine, 2-Chloro-6-[5-(3,5-dichloro-phenyl)-5-trifluoromethyl-2,5-dihydro-furan-3-yl]-N-(1,1-dioxo-1lambda*6*-thietan-3-yl)-nicotinamide (compound F2 from Table F) could be prepared. ¹H-NMR (CDCl₃, 400 MHz): 8.20 (d, 1H, J=8.07 Hz), 7.50-7.46 (m, 3H), 7.40-7.37 (m, 2H), 6.82 (m, 1H), 5.40 (dd, J=2.2 and 13.2 Hz, 1H), 5.28 (bd, 1H, J=13.2 Hz), 4.96-4.90 (m, 1H), 4.67-4.61 (m, 2H), 4.12-4.09 (m, 2H) ppm. ¹⁹F-NMR (CDCl₃, 376 MHz): −77.87 ppm.

### Example P7

### 4-[5-(3,5-Dichloro-phenyl)-5-trifluoromethyl-2,5-dihydro-furan-3-yl]-2-methyl-benzamide

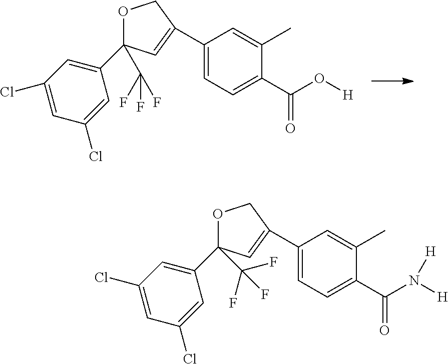 [SMILES: Cc1cc(C2=CC(c3cc(Cl)cc(Cl)c3)(C(F)(F)F)OC2)ccc1C(=O)O ; Cc1cc(C2=CC(c3cc(Cl)cc(Cl)c3)(C(F)(F)F)OC2)ccc1C(N)=O]

A solution of 4-[5-(3,5-Dichloro-phenyl)-5-trifluoromethyl-2,5-dihydro-furan-3-yl]-2-methyl-benzoic acid (330 mg) was suspended in dichloromethane (4 mL). A catalytic amount of N,N-dimethylformamide (“DMF”) and oxalyl chloride (0.08 mL) were added to the suspension. The reaction mixture was stirred at ambient temperature for one hour. The reaction mixture was concentrated and the residue dissolved in dry tetrahydrofuran (4 mL). To the solution was added a solution of ammonium hydroxide (2 mL, 25%). The reaction mixture was stirred at ambient temperature for one hour. It was then quenched by addition of water and extracted with ethyl acetate. The organic extract was washed with water, dried over sodium sulfate and concentrated in vacuo. The crude residue was treated with Diisopropylether (ca 0.1 mL) and pentane (1 mL). The mixture was stirred at room temperature for 30 minutes then the precipitate was filtered, washed twice with pentane and dried under vacuo to give the title compound (308 mg) as a bright red solid. ¹H-NMR (CDCl₃, 400 MHz): 7.49 (m, 3H), 7.48 (m, 1H), 7.27-7.21 (m, 2H), 6.38 (s, 1H), 5.74 (bs, 2H), 5.32 (dd, J=2.2 and 12.4 Hz, 1H), 5.20 (bd, 1H, J=12.2 Hz), 2.53 (s, 3H) ppm. ¹⁹F-NMR (CDCl₃, 376 MHz): −78.14 ppm.

### Example P8

### (Compound A86 from Table A): (E)-4-[5-(3,5-Dichlorophenyl)-5-trifluoromethyl-2,5-dihydro-furan-3-yl]-N-[(methoxyimino)methyl]-2-methylbenzamide

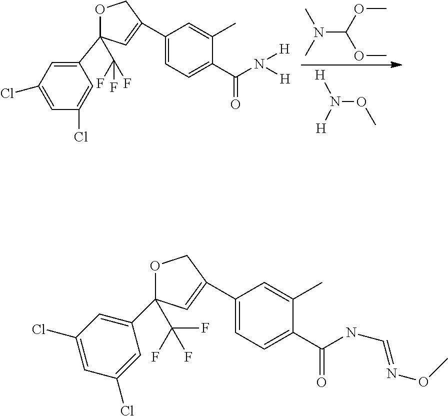

A solution of 4-[5-(3,5-Dichloro-phenyl)-5-trifluoromethyl-2,5-dihydro-furan-3-yl]-2-methyl-benzamide (100 mg) and N,N-Dimethylformamide dimethylacetal (4.2 mL) was refluxed under Argon for 30 min then the solution was concentrated in vacuo. The residue was dissolved in 1,4-dioxane (2 mL) and a solution of methoxyamin hydrochloride (56 mg) and sodium hydroxide (58 mg) in water (1.6 mL) and acetic acid (1.6 mL) was added. The solution was stirred at rt for one hour. It was then quenched by addition of water and extracted with methyl tert-butyl ether. The combined organic extracts were washed with brine, dried (Na₂SO₄) and evaporated.

Flash Chromatography eluting with Cyclohexane:EtOAc afforded 78 mg of the title compound. ¹H-NMR (CDCl₃, 400 MHz): 8.5 (m, 1H), 7.8 (m, 1H), 7.55-7.48 (m, 3H), 7.40 (m, 1H), 7.32-7.25 (m, 3H), 6.42 (m, 1H), 5.32 (dd, 1H), 5.20 (bd, 1H), 3.9 (s, 3H), 2.53 (s, 3H) ppm.

### Example P9

### (Compound D24 from Table D): 4-[5-(3,5-Dichloro-phenyl)-5-trifluoromethyl-4,5-dihydro-furan-3-yl]-N-(1,1-dioxo-1lambda*6*-thietan-3-yl)-2-methyl-benzamide

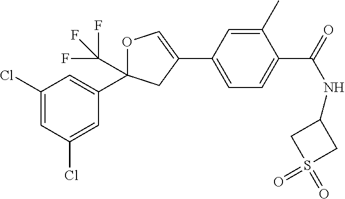 [SMILES: Cc1cc(C2=COC(c3cc(Cl)cc(Cl)c3)(C(F)(F)F)C2)ccc1C(=O)NC1CS(=O)(=O)C1]

### Step A: 2-(3,5-Dichloro-phenyl)-1,1,1-trifluoro-pent-4-yn-2-ol

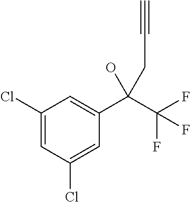 [SMILES: C#CCC(O)(c1cc(Cl)cc(Cl)c1)C(F)(F)F]

n-BuLi (15.7 mL, 1.5 equiv) was added dropwise to a solution of diisopropylamine (2.62 mL, 1.5 equiv) in tetrahydrofuran (40 mL) at 0° C. under argon. The reaction mixture was stirred for 10 min, and cooled down to −20° C. Then trimethylsilylpropyne (2.21 mL, 1.2 equiv) was added dropwise. The reaction mixture was then stirred at −20° C. for 90 minutes. The reaction mixture was cooled down to −78° C., and 1-(3,5-Dichloro-phenyl)-2,2,2-trifluoroethanone (3 g, 12.35 mmol) in tetrahydrofuran (20 mL) was added dropwise. The reaction mixture was allowed to warm up to room temperature and stirred for two hours 30 minutes. It was then quenched by addition of saturated aqueous ammonium chloride and extracted with ethyl acetate. The combined organic extracts were washed with brine, dried (Na₂SO₄) and evaporated. This crude residue was then redissolved in methanol (60 mL) and treated with potassium carbonate (3.4 g, 2 equiv). The reaction mixture was stirred for 3 h and then filtered and evaporated. Flash Chromatography eluting with Cyclohexane:EtOAc (9/1) afforded 1.896 g (54%) of the title compound.

¹H-NMR (CDCl₃, 400 MHz): 7.51-7.468 (m, 2H), 7.45-7.38 (m, 1H), 3.18-2.96 (m, 3H), 2.16-2.11 (m, 1H) ppm. ¹⁹F-NMR (CDCl₃, 376 MHz): −79.02 ppm.

### Step B: 5-(3,5-Dichloro-phenyl)-5-trifluoromethyl-dihydrofuran-3-one

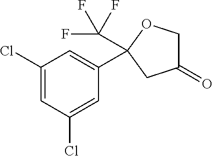 [SMILES: O=C1COC(c2cc(Cl)cc(Cl)c2)(C(F)(F)F)C1]

5-Bromo-1-oxy-nicotinic acid methyl ester (3.00 g, 1.5 equiv) (prepared according to AstraZeneca SB; AstraZeneca UK Ltd Patent WO2005/26149), triflimide (2.66 g, 1.1 equiv) and Ph₃PAuNTf₂ (338 mg, 2.5 mol %) were added in this order to a solution of 2-(3,5-Dichloro-phenyl)-1,1,1-trifluoro-pent-4-yn-2-ol (2.44 g, 8.6 mmol) in 1,2-dichloroethane (86 mL) under argon. The reaction mixture was left to stir at rt overnight. It was then concentrated in vacuo. Column chromatography eluting with cyclohexane:ethyl acteate (92/8) afforded 952 mg of the expected compound (37%).

¹H-NMR (CDCl₃, 400 MHz): 7.46-7.40 (m, 3H), 4.37 (d, 1H), 4.24 (d, 1H), 3.26 (d, 1H), 2.87 (d, 1H) ppm. ¹⁹F-NMR (CDCl₃, 376 MHz): −79.42 ppm.

### Step C: 4-[5-(3,5-Dichloro-phenyl)-3-hydroxy-5-trifluoromethyl-tetrahydro-furan-3-yl]-2-methyl-benzoic acid tert-butyl ester

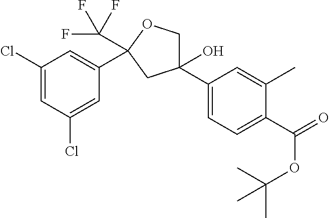 [SMILES: Cc1cc(C2(O)COC(c3cc(Cl)cc(Cl)c3)(C(F)(F)F)C2)ccc1C(=O)OC(C)(C)C]

To a solution of 4-Bromo-2-methyl-benzoic acid tert-butyl ester (234 mg, 1.2 equiv) under argon at −100° C. in tetrahydrofuran (2.6 mL) was added n-BuLi (0.62 mL, 1.2 equiv) dropwise over 5 min. The reaction mixture was left to stir for 20 min at −100° C. A solution of 5-(3,5-Dichloro-phenyl)-5-trifluoromethyl-dihydrofuran-3-one (215 mg, 0.72 mmol) in 1.0 mL tetrahydrofurane was then added dropwise. It was stirred at this temperature for one hour. The reaction mixture was quenched with water and extracted with ethyl acetate. The combined organic extracts were then washed with brine, dried (Na₂SO₄) and evaporated. Column chromatography eluting with cyclohexane:ethyl acteate (9/1) afforded 62 mg (17%) of the expected compound.

¹H-NMR (CDCl₃, 400 MHz): 7.77 (d, 1H), 7.52-7.32 (m, 3H), 7.22 (s, 1H), 7.15 (dd, 1H), 4.26 (d, 1H), 4.00 (d, 1H), 3.16 (dd, 1H), 2.95 (s, 1H), 2.81 (d, 1H), 2.54 (s, 3H), 1.58 (bs, 9H) ppm.

### Step D: 4-[5-(3,5-Dichloro-phenyl)-5-trifluoromethyl-4,5-dihydro-furan-3-yl]-2-methylbenzoic acid tert-butyl ester

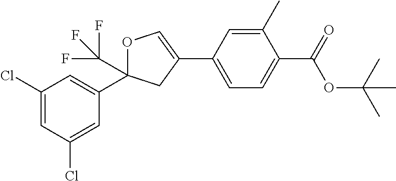 [SMILES: Cc1cc(C2=COC(c3cc(Cl)cc(Cl)c3)(C(F)(F)F)C2)ccc1C(=O)OC(C)(C)C]

A solution of 4-[5-(3,5-Dichloro-phenyl)-3-hydroxy-5-trifluoromethyl-tetrahydro-furan-3-yl]-2-methyl-benzoic acid tert-butyl ester (62 mg, 0.12 mmol) in dichloromethane (1.2 mL) under argon at −78° C. was treated with thionyl chloride (26 μL, 3 equiv) followed by triethylamine (0.13 mL, 7.5 equiv). The reaction mixture was stirred at −78° C. until complete consumption of starting material. It was quenched with water and extracted with EtOAc. The combined organic extracts were then washed with brine, dried (Na₂SO₄) and evaporated. Column chromatography eluting with cyclohexane:ethyl acetate (95/5) afforded a mixture of the title compound and its isomer in a 3:2 ratio.

### Step D′: 4-[5-(3,5-Dichloro-phenyl)-5-trifluoromethyl-4,5-dihydro-furan-3-yl]-2-ethylbenzoic acid tert-butyl ester

A solution of trimethylsilyldiazomethane (0.74 mL, 2.0 M in Et2O, 2.2 equiv) in dimethoxyethane (4 mL) under argon at −78° C. was treated with MeLi (0.92 mL, 1.6 M in Et₂O, 2.2 equiv). After 15 min at −78° C., a solution of 4-[3-(3,5-dichlorophenyl)-4,4,4-trifluoro-3-hydroxybutyryl]-2-methylbenzoic acid tert-butyl ester (321 mg, 0.67 mmol) in dimethoxyethane (2+0.7 mL) was added to the reaction mixture, which was stirred at −78° C. for one hour and then warmed up to room temperature. It was left to stir at this temperature for 2 h. It was then quenched by addition of AcOH (85 μL, 2.2 equiv) and a 1M solution of TBAF in THF was added (2 mL, 3 equiv). The reaction mixture was left to stir overnight. It was quenched by addition of water and extracted with ethyl acetate. The combined organic layers were dried (Na₂SO₄) and evaporated. Flash Chromatography eluting with cyclohexane:ethyl acetate (98/2) afforded 85 mg of the title compound (27%) as a light yellow oil which solidifies upon standing.

¹H-NMR (CDCl₃, 400 MHz): 7.79 (d, 1H), 7.52-7.45 (m, 2H), 7.43-7.37 (m, 1H), 7.08 (dd, 1H), 7.05 (s, 1H), 7.01 (s, 1H), 3.74 (dd, 1H), 3.31 (dd, 1H), 2.56 (s, 3H), 1.60 (bs, 9H) ppm. ¹⁹F-NMR (CDCl₃, 376 MHz): −80.85 ppm.

Similarly, 4-[2-(3,5-dichlorophenyl)-2-(trifluoromethyl)-3H-furan-4-yl]-2-methyl-benzonitrile could be obtained when 4-[3-(3,5-dichlorophenyl)-4,4,4-trifluoro-3-hydroxy-butanoyl]-2-methyl-benzonitrile was used as a starting material.

¹H-NMR (CDCl₃, 400 MHz): δ=7.53 (d, 1H), 7.45-7.50 (m, 2H), 7.37-7.45 (m, 1H), 7.10-7.18 (m, 2H), 7.02-7.10 (m, 1H), 3.73 (dd, 1H), 3.25-3.38 (m, 1H), 2.48-2.54 (m, 3H) ppm. ¹⁹F-NMR (CDCl₃, 376 MHz): −80.88 ppm.

Similarly, 4-(4-bromo-3-methyl-phenyl)-2-(3,5-dichlorophenyl)-2-(trifluoromethyl)-3H-furan could be obtained when 1-(4-bromo-3-methyl-phenyl)-3-(3,5-dichlorophenyl)-4,4,4-trifluoro-3-hydroxy-butan-1-one was used as a starting material.

¹H-NMR (CDCl₃, 400 MHz): δ=7.43-7.54 (m, 4H), 7.41 (t, 1H), 7.09 (d, 1H), 6.83-6.96 (m, 2H), 3.71 (dd, 1H), 3.27 (d, 1H), 2.39 (s, 3H) ppm

¹⁹F-NMR (CDCl₃, 376 MHz): −80.82 ppm

### Step E: 4-[5-(3,5-Dichloro-phenyl)-5-trifluoromethyl-4,5-dihydro-furan-3-yl]-2-methyl-benzoic acid

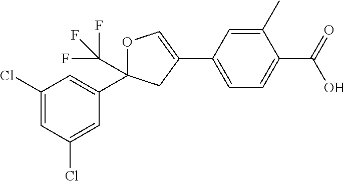 [SMILES: Cc1cc(C2=COC(c3cc(Cl)cc(Cl)c3)(C(F)(F)F)C2)ccc1C(=O)O]

A solution of 4-[5-(3,5-Dichloro-phenyl)-5-trifluoromethyl-4,5-dihydro-furan-3-yl]-2-methyl-benzoic acid tert-butyl ester (35 mg, 0.074 mmol) in dichloromethane (0.4 mL) at room temperature was treated with trifluoroacetic acid (0.055 mL, 10 equiv). The reaction mixture was stirred for 6 hours. The volatiles were removed in vacuo. Flash Chromatography eluting with cyclohexane:ethyl acetate (7/3) afforded 23 mg of the title compound (74%).

¹H-NMR (CDCl₃, 400 MHz): 8.03 (d, 1H), 7.52-7.46 (m, 2H), 7.45-7.39 (m, 1H), 7.16 (dd, 1H), 7.11 (s, 1H), 7.08 (s, 1H), 3.77 (dd, 1H), 3.33 (dd, 1H), 2.65 (s, 3H) ppm. ¹⁹F-NMR (CDCl₃, 376 MHz): −80.82 ppm.

### Step F: 4-[5-(3,5-Dichloro-phenyl)-5-trifluoromethyl-4,5-dihydro-furan-3-yl]-N-(1,1-dioxo-1lambda*6*-thietan-3-yl)-2-methyl-benzamide

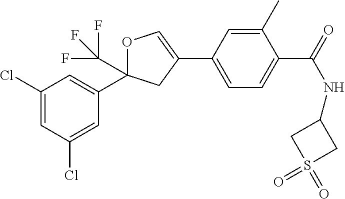 [SMILES: Cc1cc(C2=COC(c3cc(Cl)cc(Cl)c3)(C(F)(F)F)C2)ccc1C(=O)NC1CS(=O)(=O)C1]

4-[5-(3,5-Dichloro-phenyl)-5-trifluoromethyl-4,5-dihydro-furan-3-yl]-2-methyl-benzoic acid (23 mg, 0.055 mmol) was dissolved in dichloromethane (0.6 mL), and oxalyl chloride (22 μL, 5 equiv) was added. One drop of dimethylforamide was added as a catalyst, and the reaction mixture was stirred at room temperature for 18 hours. Volatiles were evaporated to give the expected acid chloride. The residue was dissolved in dichloromethane. Triethylamine (17 μL, 2.2 equiv) followed by the trifluoroacetate salt of 1,1-Dioxo-1lambda*6*-thietan-3-ylamine (14 mg, 1.05 equiv) were added. The reaction mixture was then stirred at room temperature for 5 hours. The reaction was quenched by adding water and extracted with ethyl acetate. The combined organic layers were washed with brine, dried (Na₂SO₄) and evaporated. Flash Chromatography eluting with cyclohexane:ethyl acetate (7/3) afforded the title compound.

¹H-NMR (CDCl₃, 400 MHz): 7.51-7.47 (m, 2H), 7.42 (t, 1H), 7.36 (d, 1H), 7.13-7.07 (m, 2H), 7.03-6.98 (m, 1H), 6.46 (d, 1H), 4.94-4.74 (m, 1H), 4.63-4.58 (m, 2H), 4.07-3.98 (m, 2H), 3.74 (dd, 1H) 3.31 (dd, 1H), 2.46 (s, 3H) ppm. ¹⁹F-NMR (CDCl₃, 376 MHz): −80.87 ppm. m.p.=211-213° C.

Similarly, 4-[2-(3,5-dichlorophenyl)-2-(trifluoromethyl)-3H-furan-4-yl]-2-methyl-N-[(4R)-3-oxo-2-(2,2,2-trifluoroethyl)isoxazolidin-4-yl]benzamide (D25) could be prepared. ¹H NMR (CDCl₃, 400 MHz): δ=7.44-7.56 (m, 2H), 7.35-7.44 (m, 2H), 7.03-7.13 (m, 2H), 7.01 (s, 1H), 6.33 (d, J=4.4 Hz, 1H), 4.99-5.07 (m, 1H), 4.93 (d, J=4.4 Hz, 1H), 4.01-4.31 (m, 3H), 3.74 (dd, J=15.0, 2.2 Hz, 1H), 3.30 (d, J=15.8 Hz, 1H), 2.47 ppm (s, 3H).

Similarly, 4-[2-(3,5-dichlorophenyl)-2-(trifluoromethyl)-3H-furan-4-yl]-2-methyl-N-[(4R)-2-ethyl-3-oxo-isoxazolidin-4-yl]-2-methyl-benzamide (D26) could be prepared. ¹H NMR (CDCl₃, 400 MHz): δ=7.49 (d, J=1.5 Hz, 2H), 7.42 (dt, J=3.9, 2.2 Hz, 2H), 7.03-7.14 (m, 2H), 7.00 (s, 1H), 6.38 (d, J=3.7 Hz, 1H), 4.94-5.04 (m, 1H), 4.04 (dd, J=11.0, 8.4 Hz, 1H), 3.54-3.78 (m, 3H), 3.32 (d, 1H), 2.47 (s, 3H), 1.27 ppm (t, J=7.2 Hz, 4H).

### Example P10

### 4-(3,5-Dichloro-phenyl)-5,5,5-trifluoro-4-hydroxy-pent-2-ynoic acid methyl ester

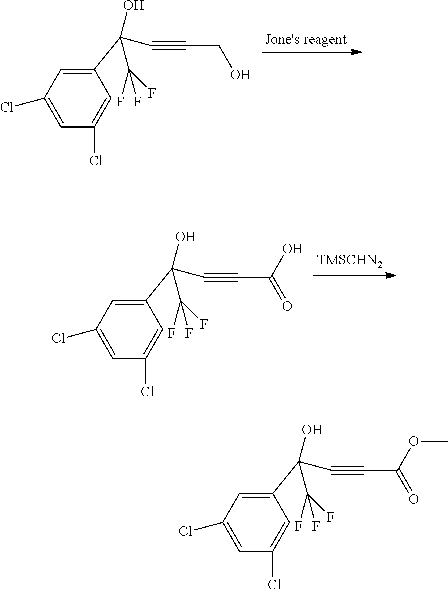 [SMILES: OCC#CC(O)(c1cc(Cl)cc(Cl)c1)C(F)(F)F ; O=C(O)C#CC(O)(c1cc(Cl)cc(Cl)c1)C(F)(F)F ; COC(=O)C#CC(O)(c1cc(Cl)cc(Cl)c1)C(F)(F)F]

### Step A:

To a solution of 4-(3,5-Dichloro-phenyl)-5,5,5-trifluoro-pent-2-yne-1,4-diol (1.8 g) in acetone (90 mL) at 0° C. was slowly added a solution of Jones' reagent (9 mL), under argon. The solution became brown and after 90 minutes, more Jone's reagent (0.8 mL) was added at 0° C. The solution was stirred for another 4 hours then it was diluted with water. The mixture was extracted with ethyl acetate, washed with a solution of sodium metabisulfite, dried over magnesium sulphate and concentrated to give 4-(3,5-Dichloro-phenyl)-5,5,5-trifluoro-4-hydroxy-pent-2-ynoic acid (1.839 g) as a colorless oil. ¹⁹F-NMR (CDCl₃, 376 MHz): −79.61 ppm.

### Step B:

To a stirring solution of 4-(3,5-Dichloro-phenyl)-5,5,5-trifluoro-4-hydroxy-pent-2-ynoic acid (1.345 g) in toluene/MeOH (27 and 9 mL) under argon at 0° C. was slowly added a solution of TMSCHN2 (3.3 mL, 2M in hexane). The reaction was exothermic and some gas evolution was observed. The solution was stirred for 4 hours at 0° C. then acetic acid (2 mL) was added. The solvent was then evaporated in vacuo to give a crude residue. The residue was purified by chromatography on silica gel (eluent: heptane/ethyl acetate 1:0 to 8:2) to give 4-(3,5-Dichloro-phenyl)-5,5,5-trifluoro-4-hydroxy-pent-2-ynoic acid methyl ester (1.2 g) as a white solid. ¹H-NMR (CDCl₃, 400 MHz): 7.60 (m, 2H), 7.46 (s, 1H), 3.87 (s, 3H) ppm.

### Example P11

### 2-Methyl-4-(boronic acid)-benzoic acid tert-butyl ester

### Steps 1 and 2:

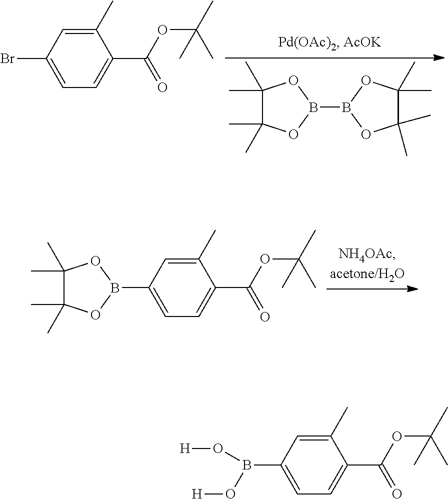

### Step A:

To a solution of 4-Bromo-2-methyl-benzoic acid tert-butyl ester (5 g) in DMF (70 mL) then 4,4,5,5,4′,4′,5′,5′-Octamethyl-[2,2′]bi[[1,3,2]dioxaborolanyl] (4.68 g), potassium acetate (5.43 g) and palladium acetate (124 mg) were successfully added. The reaction mixture was stirred at room temperature for 2 days then more palladium acetate (124 mg) was added and the mixture was stirred at 80° C. After 6 hours, more palladium acetate (124 mg) was added and the reaction mixture was stirred at 80° C. for 18 hours. Then 4,4,5,5,4′,4′,5′,5′-Octamethyl-[2,2′]bi[[1,3,2]dioxaborolanyl] (2.35 g), potassium acetate (2.7 g) and palladium acetate (124 mg) were added again to the reaction mixture. After stirring at 80° C. for 6 hours, more palladium acetate (250 mg) was added and the reaction mixture was stirred at 80° C. for 18 hours. The reaction mixture was then allowed to cool to room temperature and was diluted with ethyl acetate. The organic layer was washed with brine and HCl 1 N. It was then dried over magnesium sulfate, filtered, and concentrated in vacuo to give a crude residue that was used as such in the following step.

### Step B:

To a solution of 2-Methyl-4-(4,4,5,5-tetramethyl-[1,3,2]dioxaborolan-2-yl)-benzoic acid tert-butyl ester (1.6 g) in water/acetone (16/32 mL) at room temperature, was added ammonium acetate (1.02 g) then sodium periodate (3.1 g). The reaction was stirred at rt for 5 hours then was diluted with ethyl acetate and 1 N HCl solution. The aqueous solution was extracted with ethyl acetate. The combine organic solutions were dried with sodium sulfate, filtered, and concentrated in vacuo. The crude residue was purified by chromatography on silica gel (eluent: heptane\ethyl acetate, 1:1) to give 2-Methyl-4-(boronic acid)-benzoic acid tert-butyl ester (750 mg) as a white solid. ¹H-NMR (CDCl₃, 400 MHz): 7.82 (d, J=7.70 Hz, 1H), 7.59-7.57 (m, 2H), 2.59 (s, 3H), 1.60 (s, 9H) ppm.

### Example P12

### 4-[5-(3,5-Dichloro-phenyl)-2-oxo-5-trifluoromethyl-2,5-dihydro-furan-3-yl]-2-methyl-benzoic acid tert-butyl ester

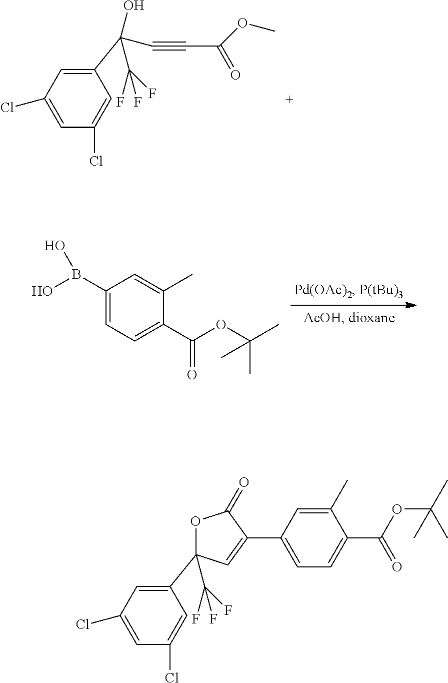 [SMILES: COC(=O)C#CC(O)(c1cc(Cl)cc(Cl)c1)C(F)(F)F ; Cc1cc(B(O)O)ccc1C(=O)OC(C)(C)C ; Cc1cc(C2=CC(c3cc(Cl)cc(Cl)c3)(C(F)(F)F)OC2=O)ccc1C(=O)OC(C)(C)C]

To a solution of 4-(3,5-Dichloro-phenyl)-5,5,5-trifluoro-4-hydroxy-pent-2-ynoic acid methyl ester (690 mg) and 2-Methyl-4-(boronic acid)-benzoic acid tert-butyl ester (750 mg) in 1,4-dioxane (10 mL) under argon, was successively added acetic acid (0.01 mL), palladium acetate (16 mg) and tri(tert-butyl)phosphine (0.03 mL). The reaction was refluxed under argon for 1 hour then stirred at room temperature for 18 hours. Then more palladium acetate (17 mg), tri(tert-butyl)phosphine (0.03 mL) and acetic acid (0.04 mL) were added. The reaction was refluxed for 4 hours then more palladium acetate (20 mg) was added. A Tricyclohexylphosphine solution (0.18 mL, 20 wt. % in toluene) was added and the solution was refluxed for 45 min. More 2-Methyl-4-(boronic acid)-benzoic acid tert-butyl ester (200 mg) was then added and the reaction was stirred at room temperature for 18 hours. It was then refluxed for 2 hours and then allowed to cool to room temperature. The reaction was concentrated under vacuo. The crude residue was purified by chromatography on silica gel (eluent: heptanes\dichloromethane, 7:3) to give 4-[5-(3,5-Dichloro-phenyl)-2-oxo-5-trifluoromethyl-2,5-dihydro-furan-3-yl]-2-methyl-benzoic acid tert-butyl ester (499 mg) as an colorless oil. ¹H-NMR (CDCl₃, 400 MHz): 7.89 (d, J=8.07 Hz, 1H), 7.80 (s, 1H), 7.76 (m, 2H), 7.54 (m, 2H), 7.49 (m, 1H), 2.62 (s, 3H), 1.61 (s, 9H).

### Example P13

### 4-[5-(3,5-Dichloro-phenyl)-2-oxo-5-trifluoromethyl-2,5-dihydro-furan-3-yl]-2-methyl-benzoic acid methyl ester

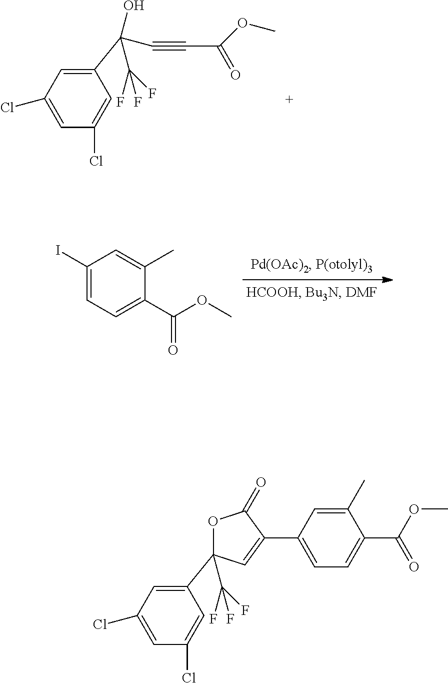 [SMILES: COC(=O)C#CC(O)(c1cc(Cl)cc(Cl)c1)C(F)(F)F ; COC(=O)c1ccc(I)cc1C ; COC(=O)c1ccc(C2=CC(c3cc(Cl)cc(Cl)c3)(C(F)(F)F)OC2=O)cc1C]

To a solution of 4-(3,5-Dichloro-phenyl)-5,5,5-trifluoro-4-hydroxy-pent-2-ynoic acid methyl ester (200 mg) and tributylamine (0.49 mL) in DMF (1.2 mL) under argon, was successively added 4-Iodo-2-methyl-benzoic acid methyl ester (404 mg), palladium acetate (14 mg), Tri(o-tolyl)phosphine (36 mg). After 30 min, formic acid (0.06 mL) was added. The reaction was stirred under argon then heated at 70 C for one hour. The reaction was quenched by addition of water and EtOAc. The mixture was extracted with diethyl ether, water, dried over magnesium sulfate, filtered and concentrated under vacuo. The crude residue was purified by chromatography on silica gel (eluent: heptanes\diethyl ether, 7:3) to give 4-[5-(3,5-Dichloro-phenyl)-2-oxo-5-trifluoromethyl-2,5-dihydro-furan-3-yl]-2-methyl-benzoic acid methyl ester (84 mg) as an orange oil. ¹H-NMR (CDCl₃, 400 MHz): 7.99 (d, J=8.80 Hz, 1H), 7.83 (s, 1H), 7.76 (m, 2H), 7.54 (m, 2H), 7.49 (m, 1H), 3.93 (s, 3H), 2.66 (s, 3H) ppm.

### Example P14

### Preparation of 2-Chloro-6-iodo-nicotinic acid methyl ester

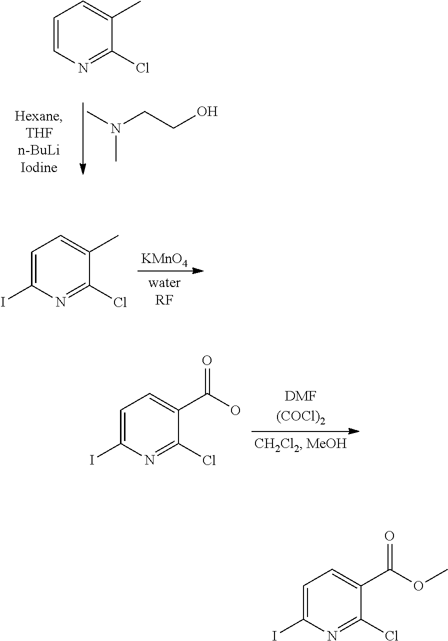

### Step A:

2-(Dimethylamino)-ethanol (20.5 mL) was dissolved in Hexane (150 mL) and the solution was stirred under argon and cooled to −5° C. n-Butyl Lithium (184 mL, 2.5 M in hexanes) was added dropwise at −5° C. and further stirred at 0° C. for 45 min. The solution was then cooled to −75° C. and a solution of 2-Chloro-3-methyl-pyridine (9.78 g) in hexane (150 mL) was added dropwise. The orange solution was stirred at −75° C. for two hours then a solution of iodine (78 g) in tetrahydrofuran (540 mL) was added dropwise at −75° C. After stirring for 2 hours, the solution was allowed to warm slowly at room temperature and stirred over night. The reaction mixture was then cooled to 0° C. and 50 ml water was added dropwise added. The solution was then extracted with diethyl ether. The organic layers were combined, dried over magnesium sulfate, filtered and concentrated under vacuo. The crude residue was purified by chromatography on silica gel (eluent: heptane\dichloromethane, 3:1) to give 2-Chloro-6-iodo-3-methyl-pyridine (16 g) as a brown solid. ¹H-NMR (CDCl₃, 400 MHz): 7.53 (d, J=7.70 Hz, 1H), 7.19 (d, J=7.34 Hz, 1H), 2.33 (s, 3H) ppm.

### Step B:

2-Chloro-6-iodo-3-methyl-pyridine (12 g) was suspended in water (250 mL) then potassium permanganate (18 g) was added. The solution was refluxed for 18 hours then the mixture was cooled to room temperature and filtered through Hyflo. The mixture was extracted with methyl tert-butyl ether. The aqueous layer was then acidified with HCl 1 M to pH 2 and extracted with ethyl acetate. The organic layers were combined, dried over magnesium sulfate, filtered and concentrated to give 2-Chloro-6-iodo-nicotinic acid (4.32 g) as a white solid. ¹H-NMR (CD₃OD, 400 MHz): 7.91 (d, J=7.70 Hz, 1H), 7.87 (d, J=8.07 Hz, 1H) ppm.

### Step C:

2-Chloro-6-iodo-nicotinic acid (3 g) was suspended in dichloromethane (40 ml). A catalytic amount of N,N-dimethylformamide (“DMF”) and oxalyl chloride (1.1 ml) were added to the suspension. The reaction mixture was stirred at ambient temperature for 1.5 hours then methanol (2 mL) was added. The reaction mixture was stirred for 30 minutes than water was added to the reaction. The mixture was extracted with methyl tert-butyl ether. The organic extract was washed with brine, dried over sodium sulfate and concentrated to give 2-Chloro-6-iodo-nicotinic acid methyl ester (3.1 g) as a brown solid. ¹H-NMR (CDCl₃, 400 MHz): 7.78 (m, 2H), 3.96 (s, 3H) ppm.

### Example P15

### 5-[5-(3,5-Dichloro-phenyl)-5-trifluoromethyl-2,5-dihydro-furan-3-yl]-2-fluoro-benzonitrile

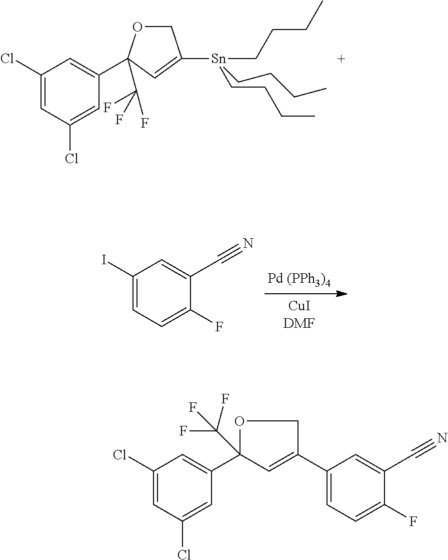 [SMILES: CCC[CH2][Sn]([CH2]CCC)([CH2]CCC)[C]1=CC(c2cc(Cl)cc(Cl)c2)(C(F)(F)F)OC1 ; N#Cc1cc(I)ccc1F ; N#Cc1cc(C2=CC(c3cc(Cl)cc(Cl)c3)(C(F)(F)F)OC2)ccc1F]

To a solution of Tributyl-[5-(3,5-dichloro-phenyl)-5-trifluoromethyl-2,5-dihydro-furan-3-yl]-stannane (300 mg) in N,N-dimethylformamide (12 mL) under argon was successively added 2-Fluoro-5-iodo-benzonitrile (630 mg), copper iodide (20 mg) and then tetrakis(triphenylphosphine) palladium (42 mg). The reaction was heated at 10° C. under argon for 20 hours. The reaction was allowed to cool down to room temperature then water was added and the mixture was extracted with ethyl acetate. The organic extract was washed with water, dried over sodium sulfate and concentrated in vacuo to give a residue which was purified by chromatography on silica gel (eluent: heptanes\dichloromethane, 2:1) to give 5-[5-(3,5-Dichloro-phenyl)-5-trifluoromethyl-2,5-dihydro-furan-3-yl]-2-fluoro-benzonitrile (182 mg) as a brown solid.

¹H-NMR (CDCl₃, 400 MHz): 7.65-7.63 (m, 2H), 7.47 (m, 2H), 7.40 (m, 1H), 7.29 (m, 1H), 6.40 (bs, 1H), 5.30 (dd, J=2.2 and 12.8 Hz, 1H), 5.17 (m, 1H) ppm. ¹⁹F-NMR (CDCl₃, 376 MHz): −78.11 and −104.17 ppm.

Similarly, 2-Chloro-6-[5-(3,5-dichloro-phenyl)-5-trifluoromethyl-2,5-dihydro-furan-3-yl]-nicotinic acid methyl ester could be prepared using 2-Chloro-6-iodo-nicotinic acid methyl ester as a coupling partner.

¹H-NMR (CDCl₃, 400 MHz): 8.21 (d, 1H, J=8.07 Hz), 7.49 (m, 2H), 7.39-7.37 (m, 2H), 6.82 (m, 1H), 5.40 (dd, J=2.57 and 13.2 Hz, 1H), 5.28 (m, 1H), 3.97 (s, 3H) ppm. ¹⁹F-NMR (CDCl₃, 376 MHz): −77.87 ppm

Similarly, 6-[5-(3,5-Dichloro-phenyl)-5-trifluoromethyl-2,5-dihydro-furan-3-yl]-2-methyl-nicotinic acid ethyl ester could be prepared using 6-Bromo-2-methyl-nicotinic acid ethyl ester as a coupling partner. ¹H-NMR (CDCl₃, 400 MHz): 8.21 (d, 1H, J=8.07 Hz), 7.50 (m, 2H), 7.38 (m, 1H), 7.31 (m, 1H), 6.73 (m, 1H), 5.42 (dd, J=2.57 and 13.2 Hz, 1H), 5.30 (m, 1H), 4.39 (q, J=6.97 Hz, 2H), 2.82 (s, 3H), 1.42 (t, J=6.97 Hz, 3H) ppm. ¹⁹F-NMR (CDCl₃, 376 MHz): −77.94 ppm

Similarly, 4-(3-Chloro-4-methyl-phenyl)-2-(3,5-dichloro-phenyl)-2-trifluoromethyl-2,5-dihydro-furan could be prepared using 2-Chloro-4-iodo-1-methyl-benzene as a coupling partner. ¹H-NMR (CDCl₃, 400 MHz): 7.48 (m, 2H), 7.37 (m, 2H), 7.25 (m, 1H), 7.16 (m, 1H), 6.31 (m, 1H), 5.28 (dd, J=2.57 and 12.47 Hz, 1H), 5.16 (m, 1H), 2.40 (s, 3H) ppm. ¹⁹F-NMR (CDCl₃, 376 MHz): −78.19 ppm.

Similarly, 5-Bromo-2-[5-(3,5-dichloro-phenyl)-5-trifluoromethyl-2,5-dihydro-furan-3-yl]-4-methyl-pyridine could be prepared using 5-Bromo-2-iodo-4-methyl-pyridine as a coupling partner. ¹H-NMR (CDCl₃, 400 MHz): 8.61 (s, 1H), 7.49 (m, 2H), 7.38 (m, 1H), 7.32 (m, 1H), 6.64 (m, 1H), 5.38 (dd, J=2.20 and 13.2 Hz, 1H), 5.27 (m, 1H), 2.43 (s, 3H) ppm. ¹⁹F-NMR (CDCl₃, 376 MHz): −78.07 ppm

Similarly, 6-[5-(3,5-Dichloro-phenyl)-5-trifluoromethyl-2,5-dihydro-furan-3-yl]-nicotinic acid methyl ester could be prepared using 6-Bromo-nicotinic acid methyl ester as a coupling partner. ¹H-NMR (CDCl₃, 400 MHz): 9.18 (m, 1H), 8.31 (m, 1H), 7.54 (m, 1H), 7.50 (m, 2H), 7.38 (m, 1H), 6.78 (m, 1H), 5.45 (dd, J=2.20 and 13.2 Hz, 1H), 5.32 (m, 1H), 3.98 (s, 3H) ppm. ¹⁹F-NMR (CDCl₃, 376 MHz): −77.94 ppm

### Example P16

### (Compound G1 from Table G): 5-[5-(3,5-Dichloro-phenyl)-5-trifluoromethyl-2,5-dihydro-furan-3-yl]-2-[1,2,4]triazol-1-yl-benzonitrile

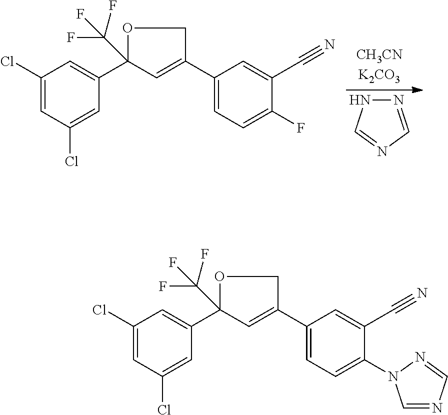 [SMILES: N#Cc1cc(C2=CC(c3cc(Cl)cc(Cl)c3)(C(F)(F)F)OC2)ccc1F ; c1nc[nH]n1 ; N#Cc1cc(C2=CC(c3cc(Cl)cc(Cl)c3)(C(F)(F)F)OC2)ccc1-n1cncn1]

To a solution of 5-[5-(3,5-Dichloro-phenyl)-5-trifluoromethyl-2,5-dihydro-furan-3-yl]-2-fluoro-benzonitrile (82 mg) in acetonitrile (3 mL) was successively added potassium carbonate (62 mg) and 1H-[1,2,4]Triazole (40 mg). The reaction was heated at 80 C for 5 hours. The reaction was allowed to cool down to room temperature then the suspension was filtered. The filtrate was concentrated under vacuo to give a residue which was purified by chromatography on silica gel (eluent: ethyl acetate\dichloromethane, 4:1) to give 5-[5-(3,5-Dichloro-phenyl)-5-trifluoromethyl-2,5-dihydro-furan-3-yl]-2-[1,2,4]triazol-1-yl-benzonitrile (74 mg) as a white foam.

¹H-NMR (CDCl₃, 400 MHz): 8.86 (bs, 1H), 8.22 (bs, 1H), 7.88 (d, J=8.44 Hz, 1H), 7.82 (bs, 1H), 7.77 (m, 1H), 7.49 (s, 2H), 7.41 (m, 1H), 6.54 (bs, 1H), 5.37 (dd, J=2.2 and 12.8 Hz, 1H), 5.24 (d, J=12.4 Hz, 1H) ppm. ¹⁹F-NMR (CDCl₃, 376 MHz): −78.00 ppm.

Similarly, 2-(4-Bromo-pyrazol-1-yl)-5-[5-(3,5-dichloro-phenyl)-5-trifluoromethyl-2,5-dihydro-furan-3-yl]-benzonitrile (compound G2 from Table G) was obtained using 4-Bromo-1H-pyrazole as a nucleophile. ¹H-NMR (CDCl₃, 400 MHz): 8.86 (bs, 1H), 8.22 (bs, 1H), 7.88 (d, J=8.44 Hz, 1H), 7.82 (bs, 1H), 7.77 (m, 1H), 7.49 (s, 2H), 7.41 (m, 1H), 6.54 (bs, 1H), 5.37 (dd, J=2.2 and 12.8 Hz, 1H), 5.24 (d, J=12.4 Hz, 1H) ppm. ¹⁹F-NMR (CDCl₃, 376 MHz): −78.03 ppm.

### Example P17

### 2-Chloro-6-[5-(3,5-dichloro-phenyl)-5-trifluoromethyl-2,5-dihydro-furan-3-yl]-nicotinic acid

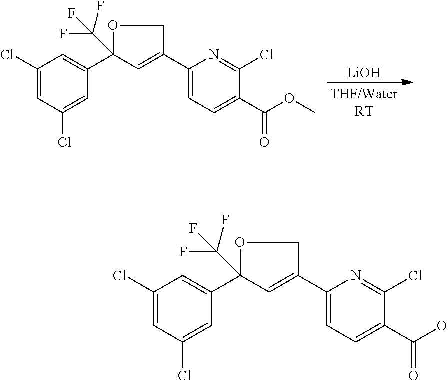

To a solution of 2-Chloro-6-[5-(3,5-dichloro-phenyl)-5-trifluoromethyl-2,5-dihydro-furan-3-yl]-nicotinic acid methyl ester (280 mg) in tetrahydrofuran/water (1.4 mL of each) was added lithium hydroxyde (30 mg). The reaction mixture was stirred at room temperature for 24 hours. The solution was then diluted by addition of water and extracted with methyl tert-butyl ether. The aqueous extract was acidified with a solution of hydrochloric acid (1M) and extracted with dichloromethane. All the organic phases were gathered, dried over sodium sulfate and concentrated in vacuo to give 4-[5-(3,5-Dichloro-phenyl)-5-trifluoromethyl-2,5-dihydro-furan-3-yl]-2-methyl-benzoic acid (190 mg) as a white foam.

⁻¹H-NMR (CDCl₃, 400 MHz): 8.37 (d, J=8.08 Hz, 1H), 7.50 (m, 2H), 7.44 (d, J=8.07 Hz, 1H), 7.39 (t, J=1.4 Hz, 1H), 6.87 (m, 1H), 5.42 (dd, J=2.2 and 13.2 Hz, 1H), 5.29 (m, 1H) ppm.

Similarly, 6-[5-(3,5-Dichloro-phenyl)-5-trifluoromethyl-2,5-dihydro-furan-3-yl]-2-methyl-nicotinic acid was obtained from the hydrolysis of 6-[5-(3,5-Dichloro-phenyl)-5-trifluoromethyl-2,5-dihydro-furan-3-yl]-2-methyl-nicotinic acid ethyl ester. ¹⁹F-NMR (CDCl₃, 376 MHz): −77.91 ppm.

Similarly, 6-[5-(3,5-Dichloro-phenyl)-5-trifluoromethyl-2,5-dihydro-furan-3-yl]-nicotinic acid was obtained from the hydrolysis of 6-[5-(3,5-Dichloro-phenyl)-5-trifluoromethyl-2,5-dihydro-furan-3-yl]-nicotinic acid methyl ester. ¹⁹F-NMR (CDCl₃, 376 MHz): −77.92 ppm.

Similarly, 4-[5-(3,5-Dichloro-phenyl)-2-oxo-5-trifluoromethyl-2,5-dihydro-furan-3-yl]-2-methyl-benzoic acid was obtained from the hydrolysis of 4-[5-(3,5-Dichloro-phenyl)-2-oxo-5-trifluoromethyl-2,5-dihydro-furan-3-yl]-2-methyl-benzoic acid methyl ester. ¹⁹F-NMR (CDCl₃, 376 MHz): −76.42 ppm.

### Example P18

### Preparation of 2-[1-[4-[2-(3,5-dichlorophenyl)-2-(trifluoromethyl)-3H-furan-4-yl]phenyl]ethyl]isoindoline-1,3-dione

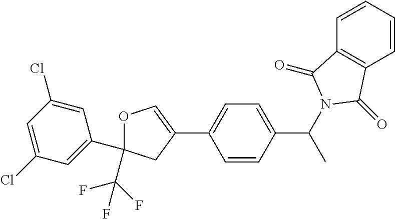 [SMILES: CC(c1ccc(C2=COC(c3cc(Cl)cc(Cl)c3)(C(F)(F)F)C2)cc1)N1C(=O)c2ccccc2C1=O]

### Step A: Preparation of 2-(3,5-dichlorophenyl)-1,1,1-trifluoro-but-3-en-2-ol

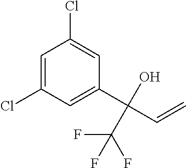 [SMILES: C=CC(O)(c1cc(Cl)cc(Cl)c1)C(F)(F)F]

Vinylmagnesium bromide 1M in THF (216.2 mL, 216.20 mmol) was added to a solution of 1-(3,5-dichlorophenyl)-2,2,2-trifluoro-ethanone (51.50 g, 211.93 mmol) in dry THF (425 mL) slowly at −75° C. to −65° C. The reaction mixture was allowed to warm to room temperature, and stirred at rt overnight. It was quenched by pouring into 2M aqueous HCl (140 mL) and extracted three times with diethyl ether. The combined organic fractions were washed successively with saturated NaHCO₃ solution, water, and brine and dried (MgSO₄). The solution was filtered and the solvent was removed under reduced pressure. Vacuum distillation (80-85° C./1 mbar) of the residue afforded 53.34 g (92.6%) of the title compound as a clear colorless liquid.

¹H-NMR (400 MHz, CDCl₃): δ 2.61 (s, 1H, OH), 5.57 (d, J=11 Hz, 1H), 5.62 (d, J=17.2 Hz, 1H), 6.36 (dd, J1=17.2 Hz, J2=11 Hz, 1H), 7.37 (t, J=1.8 Hz, 1H), 7.46-7.50 (m, 2H) ppm.

¹⁹F-NMR (377 MHz, CDCl₃): δ −78.80 ppm.

### Step B: Preparation of 5-(3,5-dichlorophenyl)-5-(trifluoromethyl)tetrahydrofuran-2-ol

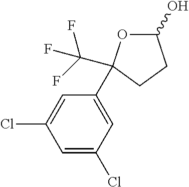 [SMILES: OC1CCC(c2cc(Cl)cc(Cl)c2)(C(F)(F)F)O1]

Rh(CO)₂acac (0.0048 g, 0.018 mmol) and 6-diphenylphosphanyl-1H-pyridin-2-one (0.026 g, 0.09 mmol) were dissolved in toluene (80 mL) under argon. 2-(3,5-dichlorophenyl)-1,1,1-trifluoro-but-3-en-2-ol (5 g, 18.45 mmol) was added and the reaction mixture was then transferred into a mechanically stirred stainless steel autoclave (300 mL). The autoclave was purged three times with hydrogen (5 bar), pressurized with hydrogen and carbon monoxide to 20 bar (CO/H₂ composition=1:1). The reaction was vigorously stirred and heated (80° C.) for 22 h. The reaction was stopped by cooling the autoclave to RT, venting and purging with argon. The reaction mixture was evaporated in vacuum and the product was isolated by column chromatography (n-heptane/AcOEt gradient) as a brown gum in 5.0 g (11.13 mmol, 60%) yield.

¹HNMR (CDCl₃, 400 MHz): δ 2.45-2.08 (m, 4H); 2.80-2.61 (m, 3H); 5.67 (d, 1H, J=4.8 Hz); 5.75 (d, 1H, J=4.8 Hz); 7.38-7.23 (m, 6H) ppm.

### Step C: Preparation of 2-(3,5-dichlorophenyl)-2-(trifluoromethyl)-3H-furan

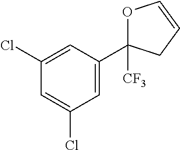 [SMILES: FC(F)(F)C1(c2cc(Cl)cc(Cl)c2)CC=CO1]

A mixture of 5-(3,5-dichlorophenyl)-5-(trifluoromethyl)tetrahydrofuran-2-ol (5 g, 11.1 mmol) and pyridinium 4-toluenesulfonate (1.68 g, 6.68 mmol) was heated to and finally distilled using a kugelrohr distillation apparatus (150° C., vacuum 100 to 4 mbar). The desired product was obtained as a white solid (2.41 g, 8.51 mmol, 76%).

¹HNMR(CDCl₃, 400 MHz): δ 2.95 (d, 1H, J=15.8 Hz); 3.40 (d, 1H, J=15.8 Hz); 5.03 (d, 1H, J=2.6 Hz); 6.43 (d, 1H, J=2.6 Hz); 7.43 (s, 2H), 7.39 (s, 1H) ppm.

### Step D: Preparation of 4,5-dibromo-2-(3,5-dichlorophenyl)-2-(trifluoromethyl)tetrahydrofuran

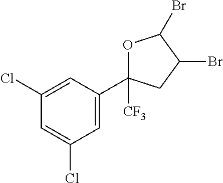 [SMILES: FC(F)(F)C1(c2cc(Cl)cc(Cl)c2)CC(Br)C(Br)O1]

A solution of bromine (1.13 g, 0.363 mL, 7.07 mmol) in dichloromethane (0.4 ml) was added to a solution of 2-(3,5-dichlorophenyl)-2-(trifluoromethyl)-3H-furan (2.00 g, 7.07 mmol) in dichloromethane (56 mL) slowly at −75° C. under argon. The reaction mixture was allowed to warm to room temperature and stirred for additional 20 minutes. Then, the reaction mixture was poured in a Na₂S₂O₃ aqueous solution and extracted twice with dichloromethane. The collected organic layers were dried (Na₂SO₄), filtered and evaporated under reduced pressure to give the title product (7.05 mmol, 3.12 g, 99%) as a white solid.

¹HNMR (CDCl₃, 400 MHz): δ 2.93 (d, 1H, J=14.7 Hz); 3.62 (dd, 1H, J=5.5 Hz, J=14.7 Hz); 4.9 (d, 1H, 5.5 Hz); 6.76 (s, 1H); 7.49 (m, 3H) ppm.

### Step E: Preparation of 4-bromo-2-(3,5-dichlorophenyl)-2-(trifluoromethyl)-3H-furan

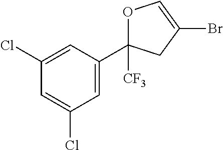 [SMILES: FC(F)(F)C1(c2cc(Cl)cc(Cl)c2)CC(Br)=CO1]

1,8-Diazabicyclo[5.4.0]undec-7-ene (0.103 g, 0.101 mL, 0.68 mmol) was dropwise added to a solution of 4,5-dibromo-2-(3,5-dichlorophenyl)-2-(trifluoromethyl)tetrahydrofuran (0.150 g, 0.34 mmol) in N,N-dimethylformamide (1 mL) at room temperature under argon. Then, the reaction mixture was warmed to 100° C. and stirred at that temperature for 20 min. The reaction mixture was quenched by pouring into a 2M HCl solution and extracted with n-hexane (3 times). The organic phase was dried (Na₂SO₄) and evaporated under reduce pressure giving the title compound (75 mg, 0.207 mmol, 61%) as a yellow oil.

¹HNMR (CDCl₃, 400 MHz): δ 3.15 (d, 1H, J=15.8 Hz); 3.56 (dd, 1H, J₁=15.8 Hz, J₂=2.6 Hz); 6.51 (t, 1H, J=2.2 Hz); 7.39 (s, 1H); 7.41 (t, 2H, J=1.5 Hz) ppm.

### Step F: Preparation of 2-[1-[4-(4,4,5,5-tetramethyl-1,3,2-dioxaborolan-2-yl)phenyl]ethyl]isoindoline-1,3-dione

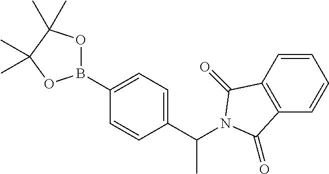 [SMILES: CC(c1ccc(B2OC(C)(C)C(C)(C)O2)cc1)N1C(=O)c2ccccc2C1=O]

Pd(DPPF)Cl₂.CH₂Cl₂ (0.03957 g, 0.048 mmol) and potassium acetate (0.48 g, 4.85 mmol) were added to a solution of 2-[1-(4-bromophenyl)ethyl]isoindoline-1,3-dione (0.8 g, 2.423 mmol) and pinacol diborane (0.738 g, 2.91 mmol) in N,N-dimethylformamide (7 mL). The reaction mixture was stirred at 90° C. for 12 h under argon. The reaction mixture was diluted with water and ethyl acetate. Organic phase was washed 4 times with water and once with brine. It was dried and concentrated in vacuum. The crude material was purified by column chromatography (n-heptane/ethyl acetate gradient). The title product was obtained as a white solid (536 mg, 1.42 mmol, 59%).

¹HNMR (CDCl₃, 400 MHz): δ 1.32 (s, 12H); 1.93 (d, 3H, J=7.3 Hz); 5.59 (q, 1H, 7.3 Hz); 7.5 (d, 2H, J=7.7 Hz); 7.93-7.76 (m, 4H) ppm.

### Step G: Preparation of 2-[1-[4-[2-(3,5-dichlorophenyl)-2-(trifluoromethyl)-3H-furan-4-yl]phenyl]ethyl]isoindoline-1,3-dione

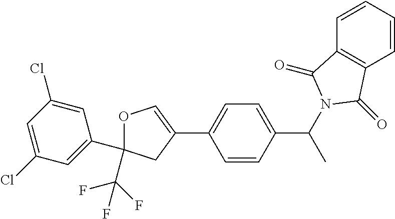 [SMILES: CC(c1ccc(C2=COC(c3cc(Cl)cc(Cl)c3)(C(F)(F)F)C2)cc1)N1C(=O)c2ccccc2C1=O]

A test tube containing a magnetic stir bar was charged with S-Phos palladacycle catalysts (CAS=1028206-58-7, STREM=46-0269) (0.018 g, 0.0026 mmol); 2-[1-[4-(4,4,5,5-tetramethyl-1,3,2-dioxaborolan-2-yl)phenyl]ethyl]isoindoline-1,3-dione (0.06232 g, 0.16 mmol) and potassium phosphate (0.055619 g, 0.26 mmol). The tube was capped with a rubber septum, evacuated and backfilled with argon (this sequence was repeated three times). Deionized water (0.02 mL) and dry toluene (0.4 mL) and were added sequentially and the resulting mixture was stirred at room temperature for ˜2 min. 4-bromo-2-(3,5-dichlorophenyl)-2-(trifluoromethyl)-3H-furan (0.046 g, 0.13 mmol) was added dropwise via syringe. The reaction mixture was stirred vigorously at 100° C. for 18 h. The reaction mixture was diluted with AcOEt, washed with water, dried (Na₂SO₄) and evaporated. The residue was purified by flash chromatography on silica gel (n-heptane/ethyl acetate gradient 9:1 to 5:5) to giving the title compound (0.034 mmol, 18 mg, 27%) as a white solid.

¹HNMR(CDCl₃, 400 MHz): δ 3.62 (dt, 1H, J₁=15.3, J₂=2.2 Hz); 5.47 (q, 1H, J=7.3 Hz); 6.83 (s, 1H); 7.11 (d, 2H, J=8.4 Hz); 7.31 (t, 1H, J=1.8 Hz); 7.37 (s, 1H); 7.39 (m, 3H); 7.61 (m, 2H); 7.72 (m, 2H) ppm.

### Example P19

### Preparation of 4-[(2S)-2-(3,5-dichlorophenyl)-2-(trifluoromethyl)-3H-furan-4-yl]-N-(1,1-dioxothietan-3-yl)-2-methyl-benzamide (compound D24*)

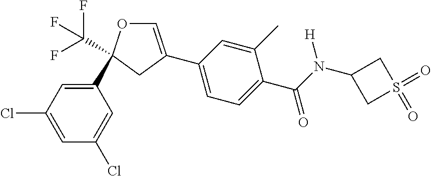 [SMILES: Cc1cc(C2=CO[C@@](c3cc(Cl)cc(Cl)c3)(C(F)(F)F)C2)ccc1C(=O)NC1CS(=O)(=O)C1]

### Step A: 2-Methyl-4-trimethylsilanylethynyl-benzoic acid tert-butyl ester

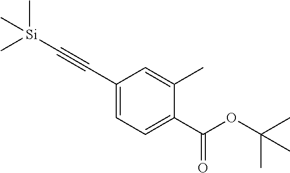 [SMILES: Cc1cc(C#C[Si](C)(C)C)ccc1C(=O)OC(C)(C)C]

To a solution of tert-butyl 4-bromo-2-methyl-benzoate (100 g, 368.8 mmol) in tetrahydrofuran (6 mL/mmol) and diisopropylamine (1.2 equiv., 442.5 mmol) at room temperature were added copper(I)-iodide (0.05 equiv., 18.44 mmol) and dichlorobis(triphenylphosphine)palladate(II) (0.05 equiv., 18.44 mmol). Argon was bubbled through the reaction for 5 minutes then ethynyl(trimethyl)silane (2.2 equiv., 811.3 mmol) was added dropwise over a 15 min period. The mixture was heated at 45° C. for 4 h. The mixture was filtered over celite and the filter cake washed with ethyl acetate. The organic phase was then washed twice with a saturated NH₄Cl solution and once with brine, dried over Na₂SO₄ and solvents were evaporated under reduced pressure. The brown oil residue was purified over a silica gel column (eluent: cyclohexane/EtOAc) to give 40 g of as a yellow oil. LCMS (Method A) 1.40 min; ¹H-NMR (CDCl₃, 400 MHz): 0.25 (s, 3H), 1.61 (s, 9H), 2.52 (s, 3H), 7.30 (s, 1H), 7.32 (d, 1H), 7.74 (d, 1H).

### Step B: 4-Ethynyl-2-methyl-benzoic acid tert-butyl ester

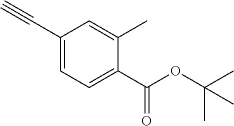 [SMILES: C#Cc1ccc(C(=O)OC(C)(C)C)c(C)c1]

To a solution of tert-butyl 2-methyl-4-(2-trimethylsilylethynyl)benzoate (103.0 g, 357.0 mmol) in methanol (500 mL) at room temperature was added potassium carbonate (75.09 g, 535.5 mmol). The resulting suspension was rapidly stirred at room temperature for 15 min and then water was added until dissolution of K₂CO₃. The mixture was extracted twice with dichloromethane. The combined organic phases were washed with brine, dried over MgSO₄ and solvent were evaporated under reduced pressure. The crude product was purified over a silica gel column (eluent: heptane/EtOAc) to give 82 g of tert-butyl 4-ethynyl-2-methyl-benzoate as a yellow oil. LCMS (Method A) RT 1.18 min; ¹H-NMR (CDCl₃, 400 MHz): 1.62 (s, 9H), 2.57 (s, 3H), 3.18 (s, 1H), 7.29 (s, 1H), 7.38 (d, 1H), 7.80 (d, 1H).

### Step C: tert-butyl 4-[(3R)-3-(3,5-dichlorophenyl)-4,4,4-trifluoro-3-hydroxy-but-1-ynyl]-2-methyl-benzoate

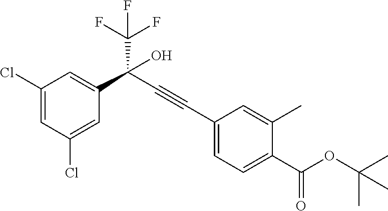 [SMILES: Cc1cc(C#C[C@](O)(c2cc(Cl)cc(Cl)c2)C(F)(F)F)ccc1C(=O)OC(C)(C)C]

To a stirred solution of quinine (0.2 equiv., 16.5 mmol), barium(2+) dihydrofluoride (0.2 equiv., 16.46 mmol) and tert-butyl 4-ethynyl-2-methyl-benzoate (2.5 equiv., 205.8 mmol) was added slowly dimethylzinc (4.0 equiv., 329.2 mmol, 2.0 mol/L) and the mixture was stirred at room temperature overnight. Tetraisopropoxytitanium (4 equiv., 329.2 mmol) was then added and stirring was continued for another 3 hours to give an orange solution. Then, the solution was treated with 1-(3,5-dichlorophenyl)-2,2,2-trifluoro-ethanone (20 g, 82.30 mmol) in one portion. The reaction mixture was stirred at room temperature for 3 days. The reaction mixture was quenched carefully with NH₄Cl sat aqueous solution at 0° C., then allowed to stir at room temperature for 20 min. The toluene phase was then filtered over celite. The aqueous phase was extracted twice with ethyl acetate and each time the organic phases were filtered over celite. Finally, the ethyl acetate phases were grouped, washed once with brine, dried over magnesium sulfate and solvents were removed under reduced pressure. The crude product was purified over a silica gel column (eluent: heptane/EtOAc) to give 33.4 g of expected tert-butyl 4-[3-(3,5-dichlorophenyl)-4,4,4-trifluoro-3-hydroxy-but-1-ynyl]-2-methyl-benzoate) as a colorless oil. LCMS (Method A) RT 1.37 min, [M+H]⁺ 457/459/460; ¹H-NMR (CDCl₃, 400 MHz): 1.60 (s, 9H), 2.55 (s, 3H), 3.64 (s, 1H, OH), 7.27 (s, 1H), 7.33 (d, 1H), 7.41 (s, 1H), 7.68 (m, 2H), 7.79 (d, 1H). ¹⁹F-NMR (CDCl₃, 376.3 MHz): −80.05.

Chiral HPLC Analysis:

Column: Daicel CHIRALPAK® IB, 3 μm, 0.46 cm×10 cm

Mobile phase: Hept/DCM 50/50

Flow rate: 1.0 ml/min

2 isomers were detected: rt=1.94 min (86.2%) 2.28 min. (13.8%).

### Step D: tert-butyl 4-[(E,3S)-3-(3,5-dichlorophenyl)-4,4,4-trifluoro-3-hydroxy-but-1-enyl]-2-methyl-benzoate

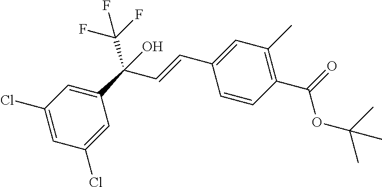 [SMILES: Cc1cc(/C=C/[C@](O)(c2cc(Cl)cc(Cl)c2)C(F)(F)F)ccc1C(=O)OC(C)(C)C]

To a solution of tert-butyl 4-[(3R)-3-(3,5-dichlorophenyl)-4,4,4-trifluoro-3-hydroxy-but-1-ynyl]-2-methyl-benzoate (43.0 g, 93.6 mmol) in 400 mL of toluene and 20 mL of THF cooled to −40° C., sodium bis(2-methoxyethoxy)aluminum hydride (70 mass % in Toluene) (Approx. 3.5 M) (2.0 equiv., 187.0 mmol) was added dropwise keeping the reaction below −30° C. (gas-evolution). The reaction was stirred at −40° C. for 1 h. The reaction mixture was carefully quenched first with acetone (10 mL) at −40° C. and then with NH₄Cl solution sat at −10° C. and extracted twice with ethyl acetate. The combined organic layers were dried (MgSO4), filtered and evaporated to give a colorless oil. The crude product was purified over a silica gel column (eluent: cyclohexane/EtOAc) to give 5.25 g of tert-butyl 4-[(E)-3-(3,5-dichlorophenyl)-4,4,4-trifluoro-3-hydroxy-but-1-enyl]-2-methyl-benzoate as a colorless oil. LCMS (Method A) RT 1.35 min, [M+H]⁺ 459/461/462; ¹H-NMR (CDCl₃, 400 MHz): 1.6 (s, 9H), 2.57 (s, 3H), 2.80 (s, 1H), 6.75 (dd, 2H), 7.25 (m, 2H), 7.49 (m, 1H), 7.53 (m, 2H), 7.8 (d, 1H). ¹⁹F-NMR (CDCl₃, 376.3 MHz): −79.4.

### Step E: 4-[(2S)-2-(3,5-dichlorophenyl)-2-(trifluoromethyl)-3H-furan-4-yl]-2-methyl-benzoic acid

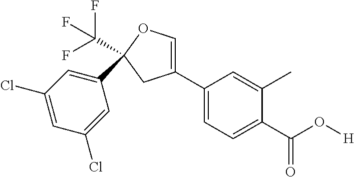 [SMILES: Cc1cc(C2=CO[C@@](c3cc(Cl)cc(Cl)c3)(C(F)(F)F)C2)ccc1C(=O)O]

A homogeneous solution of [Rh(CO)₂acac] (0.01 equiv., 0.009 mmol), tert-butyl 4-[(E)-3-(3,5-dichlorophenyl)-4,4,4-trifluoro-3-hydroxy-but-1-enyl]-2-methyl-benzoate (0.4 g, 0.88 mmol) and tris(2,4-ditert-butylphenyl)phosphite (0.1 equiv., 0.087 mmol) in toluene (8 mL) in a stainless steel autoclave was purged three times with hydrogen (5 bar), pressurized at 25 bar with H2 followed by an additional 25 bar of CO (=50 bar CO/H₂ 1:1). The reaction was then heated at 100° C. and vigorously stirred for 20 h. The reaction was stopped by cooling the autoclave to room temperature, venting and purging with argon. The crude reaction was transferred into a 30 mL vial and 4-methylbenzenesulfonic acid (0.2 equiv., 0.173 mmol) was added and mixture was heated at reflux for 5 hours. The mixture was then cooled to room temperature, diluted with ethyl acetate and washed twice with NaHCO₃ sat aqueous solution, once with water and once with brine. The organic phase was then dried over magnesium sulfate, filtered and solvents were evaporated under reduced pressure. The crude product was purified over a silica gel column (eluent: cyclohexane/EtOAc) to yield a 154 mg of 4-[2-(3,5-dichlorophenyl)-2-(trifluoromethyl)-3H-furan-4-yl]-2-methyl-benzoic acid. LCMS (Method A) RT 1.23 min; 1H-NMR (CDCl₃, 400 MHz): 2.68 (s, 3H), 3.35 (d, 1H), 3.78 (d, 1H), 7.10 (m, 2H), 7.18 (m, 1H), 7.42 (m, 1H), 7.52 (m, 2H), 8.10 (d, 1H). ¹⁹F-NMR (CDCl₃, 376.3 MHz): −79.4.

### Step F: 4-[(2S)-2-(3,5-dichlorophenyl)-2-(trifluoromethyl)-3H-furan-4-yl]-N-(1,1-dioxothietan-3-yl)-2-methyl-benzamide (compound D24*)

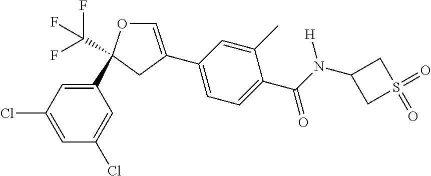 [SMILES: Cc1cc(C2=CO[C@@](c3cc(Cl)cc(Cl)c3)(C(F)(F)F)C2)ccc1C(=O)NC1CS(=O)(=O)C1]

To a stirred solution of 4-[2-(3,5-dichlorophenyl)-2-(trifluoromethyl)-3H-furan-4-yl]-2-methyl-benzoic acid (1 g, 2.40 mmol) in dry dichloromethane (50 mL) was added oxalyl chloride (1.0 equiv., 2.4 mmol) and then one drop of N,N-dimethylformamide. The reaction mixture was stirred at room temperature until no more CO formation was observed. The mixture was then evaporated to dryness and dissolved in dry dichloromethane (10 mL). This solution was then added dropwise at 0° C. to a mixture of 1,1-dioxothietan-3-amine (1.1 equiv., 2.64 mmol) and triethylamine in dry dichloromethane (20 mL). The mixture was stirred at 0° C. for 30 min and then allowed to stir at room temperature for 4 h and then quenched with water. The organic phase was washed once with brine and then solvent was evaporated under reduced pressure. The crude product was purified by flash chromatography over a silica gel column (eluent: cyclohexane/EtOAc). After removal of the solvents, a colorless oil was obtained which was dissolved in a minimum of TBME and after dilution with heptanes a white precipitate appeared which was filtered and dried under high vacuum to yield the titled compound. Mp: 90-105° C. LCMS (Method A) RT 1.16 min, [M+H]⁺ 567/569/571; ¹H-NMR (CDCl₃, 400 MHz): 1-55 (s, 2H), 2.45 (s, 3H), 3.29 (m, 1H), 3.72 (m, 1H), 4.01 (m, 2H), 4.61 (m, 2H), 4.87 (m, 1H), 6.45 (d, 1H), 7.01 (s, 1H), 7.10 (m, 2H), 7.26 (s, 1H), 7.37 (d, 1H), 7.41 (m, 1H), 7.49 (m, 2H).

¹⁹F-NMR (CDCl₃, 400 MHz): −80.87.

Chiral HPLC analysis:

Column: Daicel CHIRALPAK® IA, 3 μm, 0.46 cm×10 cm

Mobile phase: Heptan/iPrOH/DEA 80/20/0.1%

Flow rate: 1 ml/min

2 isomers were detected: rt=8.88 min (84.4%) 10.79 min. (15.6%).

### Example P20

### Preparation of 4-[(2S)-2-(3,5-dichlorophenyl)-2-(trifluoromethyl)-3H-furan-4-yl]-2-methyl-N-[2-oxo-2-(2,2,2-trifluoroethylamino)ethyl]benzamide (compound D5*)

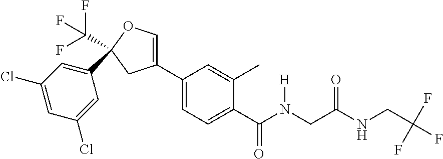 [SMILES: Cc1cc(C2=CO[C@@](c3cc(Cl)cc(Cl)c3)(C(F)(F)F)C2)ccc1C(=O)NCC(=O)NCC(F)(F)F]

4-[(2S)-2-(3,5-dichlorophenyl)-2-(trifluoromethyl)-3H-furan-4-yl]-2-methyl-N-[2-oxo-2-(2,2,2-trifluoroethylamino)ethyl]benzamide was prepared using the protocol described for the preparation of compound D24*.

Mp: 120-155° C. LCMS (Method A) RT 1.20 min; ¹H-NMR (CDCl₃, 400 MHz): 2.48 (s, 3H), 3.32 (d, 2H), 3.75 (dd, 2H), 3.95 (m, 2H), 4.22 (d, 2H), 6.67 (t, 1H), 7.02 (s, 1H), 7.12 (m, 2H), 7.28 (s, 1H), 7.40 (m, 1H), 7.43 (m, 1H), 7.51 (m, 2H). ¹⁹F-NMR (CDCl₃, 376.3 MHz): −72.46. −80.85.

Chiral HPLC analysis:

Column: Daicel CHIRALPAK® IA, 3 μm, 0.46 cm×10 cm

Mobile phase: Heptan/EtOH 80/20

Flow rate: 1 ml/min

2 isomers were detected: rt=5.56 min (84.2%) 8.14 min. (15.8%).

### Example P21

### Preparation of enantioentriched tert-butyl 4-[3-(3,5-dichlorophenyl)-4,4,4-trifluoro-3-hydroxy-but-1-ynyl]-2-methyl-benzoate

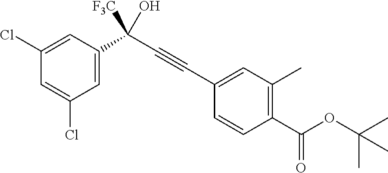 [SMILES: Cc1cc(C#C[C@@](O)(c2cc(Cl)cc(Cl)c2)C(F)(F)F)ccc1C(=O)OC(C)(C)C]

### Step A: Preparation of 2-(3,5-dichlorophenyl)-1,1,1-trifluoro-but-3-yn-2-ol

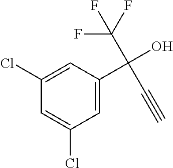 [SMILES: C#CC(O)(c1cc(Cl)cc(Cl)c1)C(F)(F)F]

In a 250 mL round bottom flask equipped with a reflux condenser, magnetic stirring and a thermometer under argon, was added bromo(ethynyl)magnesium (0.5M, 82.30 mmol). The solution was cooled to 0° C. followed by dropwise addition of 1-(3,5-dichlorophenyl)-2,2,2-trifluoro-ethanone (10 g, 41.1504 mmol) keeping the temperature under 7° C. The reaction was brought back to room temperature and was stirred overnight at room temperature. The reaction was cooled to 0° C. and quenched carefully with HCl (1M) until pH=1. The organic phase was then washed four times with water and once with brine. The combine organic phases were dried over MgSO4, filtered and solvents were evaporated under reduced pressure. The crude product was distilled under reduced pressure (1 mBar, 120° C.) to yield 9 g of a clear oil which solidified upon standing. ¹H NMR (400 MHz, CDCl₃) 2.90 (s, 1H), 3.26 (s, OH), 7.45 (d, 1H), 7.55 (d, 2H); ¹⁹F-NMR (CDCl₃, 376.3 MHz): −80.40.

### Step B: Preparation of [1-(3,5-dichlorophenyl)-1-(trifluoromethyl)prop-2-ynyl]butanoate

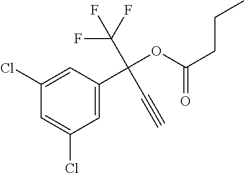 [SMILES: C#CC(OC(=O)CCC)(c1cc(Cl)cc(Cl)c1)C(F)(F)F]

In a 30 mL flask, 2-(3,5-dichlorophenyl)-1,1,1-trifluoro-but-3-yn-2-ol (2.69 g, 10 mmol), triethylamine (1.31 g, 13 mmol) and N,N-dimethylpyridin-4-amine (0.061 g, 0.5 mmol) were dissolved in dichloromethane and cooled to 0° C. Then butanoyl chloride (1.39 g, 13 mmol) was added dropwise. The mixture was stirred overnight at room temperature. The mixture was taken up in MTBE and washed with HCl (0.1 M), water, brine, dried over MgSO4, filtered on a pad of silica gel and solvents were evaporated under reduced pressure to yield 3.5 g of a clear oil. ¹H NMR (400 MHz, CDCl₃) 1.05 (m, 3H), 1.72 (m, 2H), 2.48 (m, 2H), 3.00 (s, 1H), 7.43 (d, 1H), 7.52 (d, 2H); ¹⁹F-NMR (CDCl₃, 376.3 MHz): −78.40.

### Step C: Preparation of [(1S)-1-(3,5-dichlorophenyl)-1-(trifluoromethyl)prop-2-ynyl]butanoate and (2R)-2-(3,5-dichlorophenyl)-1,1,1-trifluoro-but-3-yn-2-ol

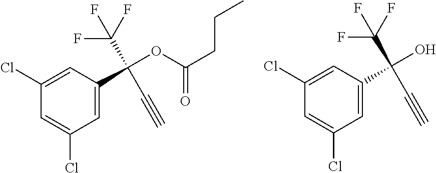 [SMILES: C#C[C@](OC(=O)CCC)(c1cc(Cl)cc(Cl)c1)C(F)(F)F ; C#C[C@@](O)(c1cc(Cl)cc(Cl)c1)C(F)(F)F]

A solution of Lipase from Candida rugosa (5 g) in phosphate buffer pH=7.4, 100 mM (100 mL) was mechanically stirred in a 250 mL glass reactor (500 rpm) at room temperature for 2 hours. Then a solution of [1-(3,5-dichlorophenyl)-1-(trifluoromethyl)prop-2-ynyl]butanoate (10 g) in DMSO (20 mL) was added to the previous solution. The reaction mixture was mechanically stirred at 55° C. (internal temperature), 500 rpm for 2 days. Aliquots were analyzed by LCMS during the course of the experiment. After 50 h, 1.742 g of K₂HPO₄ (10 mmol) was added to the mixture and stirred for a further 20 h. At this point Celite (20 g) was added and the reaction was filtered through on a Celite plug. The Celite cake was then rinsed with ethyl acetate (7×100 mL). The clear biphasic mixture was decanted and the aqueous phase was extracted with ethyl acetate (2×100 mL). The gathered organic phases were washed with brine (100 mL), dried on MgSO4, and concentrated under vacuum (40° C., 30 mbar). A viscous orange oil (m=10.80 g) was obtained. The crude product was purified by flash chromatography over a silica gel column (eluent: cyclohexane/EtOAc) to yield 4.85 g of [(1S)-1-(3,5-dichlorophenyl)-1-(trifluoromethyl)prop-2-ynyl]butanoate and 2.93 g of (2R)-2-(3,5-dichlorophenyl)-1,1,1-trifluoro-but-3-yn-2-ol.

A solution of [(1S)-1-(3,5-dichlorophenyl)-1-(trifluoromethyl)prop-2-ynyl]butanoate (108 mg in 10 mL CHCl₃) in such manner was analyzed for optical rotation at 20° C. This [α]_{D} at 20° C. was −15.37°. The use of (S)-(+)-1-(9-Anthryl)-2,2,2-trifluoroethanol enabled to determine an ee=56%.

A solution of this (2R)-2-(3,5-dichlorophenyl)-1,1,1-trifluoro-but-3-yn-2-ol (57 mg in 5 mL CHCl3) in such manner was analysed for optical rotation at 20° C. This [α]_{D} at 20° C. was +7.28°.

Chiral GC analysis:

GC was conducted on a Thermo Focus GC, with a column from Supelco Alpha DEX 120 fused silica Capillary Column: 30 m, diam: 0.25 mm, 0.25 μm, H₂ flow 1. ml/min, temp injector: 220° C., FID Detector: temp detector: 300° C., method: start at 80° C., hold 2 min, 5.5° C./min until 220° C., hold 3 min, total time 30 min

2 isomers were detected: rt=23.37 min (86.0%) 24.32 min. (14.0%).

### Step C′: Alternative preparation of enantioenriched 2-(3,5-dichlorophenyl)-1,1,1-trifluoro-but-3-yn-2-ol

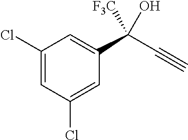 [SMILES: C#C[C@](O)(c1cc(Cl)cc(Cl)c1)C(F)(F)F]

A solution of n-butyl lithium (1.27 mL, 3.29 mmol) in hexanes (2.6M) was added to a solution of trimethylsilylacetylene (162 mg, 1.65 mmol) and (1R,2S)-1-phenyl-2-pyrrolidin-1-yl-propan-1-ol (338 mg, 1.65 mmol) in dry THF (2 mL) slowly at −10° C. The resulting solution was stirred for 30 minutes at 0° C. under argon. A solution of 1-(3,5-dichlorophenyl)-2,2,2-trifluoro-ethanone (200 mg, 0.82 mmol) in THF (2 mL) was added slowly at −40° C. and stirred at the same temperature for additional 30 minutes. After addition of HCl (1M), the reaction mixture was extracted by diethyl ether. The organic phase was dried (Na₂SO₄) and evaporated giving colorless oil (327 mg). The oily residue was solubilized in ethanol (5 mL) and treated with potassium carbonate (341 mg, 2.47 mmol) for 2 h at RT. Potassium carbonate was filtered off and the title compound was isolated by column chromatography (eluent cyclohexane/EtOAc) as a colorless liquid in 88 mg (40%) yield. ¹H NMR (400 MHz, CDCl₃) 2.90 (s, 1H), 3.26 (s, OH), 7.45 (d, 1H), 7.55 (d, 2H); ¹⁹F-NMR (CDCl₃, 376.3 MHz): −80.40.

Chiral GC analysis:

GC was conducted on a Thermo Focus GC, with a column from Supelco Alpha DEX 120 fused silica Capillary Column: 30 m, diam: 0.25 mm, 0.25 μm, H₂ flow 1. ml/min, temp injector: 220° C., FID Detector: temp detector: 300° C., method: start at 80° C., hold 2 min, 5.5° C./min until 220° C., hold 3 min, total time 30 min

2 isomers were detected: rt=23.37 min (15.0%) 24.32 min. (85.0%).

### Step D: Preparation of tert-butyl 4-[3-(3,5-dichlorophenyl)-4,4,4-trifluoro-3-hydroxy-but-1-ynyl]-2-methyl-benzoate

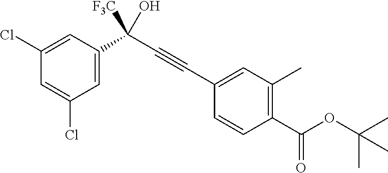 [SMILES: Cc1cc(C#C[C@@](O)(c2cc(Cl)cc(Cl)c2)C(F)(F)F)ccc1C(=O)OC(C)(C)C]

To a solution of tert-butyl 4-bromo-2-methyl-benzoate (1.1 mmol, 0.298 g) in N,N-dimethylformamide is added successively at room temperature diisopropylamine (20 equiv., 20 mmol, 2.83 ml), cooper-(I)-iodide (0.4 mmol, 78 mg) and dichlorobis(triphenylphosphine)palladate(II) (0.4 mmol, 281 mg). Argon was bubbled through the reaction for 5 minutes after which the enriched (2R)-2-(3,5-dichlorophenyl)-1,1,1-trifluoro-but-3-yn-2-ol (1.1 mmol, 0.296 g, obtained as described in step C) was added in 1 ml of DMF. The black mixture is heated under argon at 80° C. for 2 h15. The reaction mixture is filtered through celite and washed with EtOAc and water. The aqueous phase was extracted with EtOAc. The combined organic phases is washed 3 times with saturated NaCl solution. The organic phase was then dried over sodium sulfate and the solvents after filtration were evaporated under reduced pressure and filtered on a pad of silica gel to yield 40 mg of a clear oil. LCMS (method A) RT 1.37 min; ¹H-NMR (CDCl₃, 400 MHz): 1.49 (s, 9H), 2.04 (s, 3H), 3.70 (s, 1H, OH), 7.18 (m, 1H), 77.22 (m, 1H), 7.30 (m, 1H), 7.54 (m, 2H), 7.65 (d, 1H). ¹⁹F-NMR (CDCl₃, 376.3 MHz): −80.05.

Chiral HPLC analysis:

Column: Daicel CHIRALPAK® IB, 3 μm, 0.46 cm×10 cm

Mobile phase: Hept/DCM 50/50

Flow rate: 1.0 ml/min

2 isomers were detected: rt=1.94 min (22.0%) 2.28 min. (78.0%).

### Example P21

### Preparation of 5-[(2S)-2-(3,5-dichlorophenyl)-2-(trifluoromethyl)-3H-furan-4-yl]-2-(1,2,4-triazol-1-yl)benzonitrile (compound H1*)

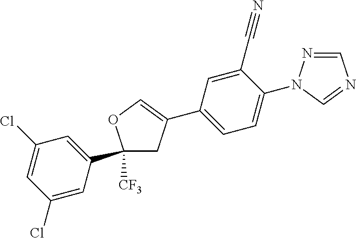 [SMILES: N#Cc1cc(C2=CO[C@@](c3cc(Cl)cc(Cl)c3)(C(F)(F)F)C2)ccc1-n1cncn1]

### Step A: Preparation of 5-iodo-2-(1,2,4-triazol-1-yl)benzonitrile

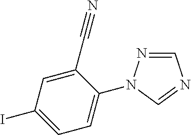 [SMILES: N#Cc1cc(I)ccc1-n1cncn1]

To a solution of 2-fluoro-5-iodo-benzonitrile (25.3 g) and 1H-1,2,4-TRIAZOLE (8.66 g) in N,N-dimethylformamide (102 mL) was added cesium carbonate (40.0 g) and the mixture was heated at 60° C. for 5 hours. The beige-brown suspension was cooled to room temperature and allowed to stand for 6 days. The mixture was dissolved in ethyl acetate, washed with a hydrochloric solution (1M). The combined organic layers were dried over magnesium sulfate, filtered and evaporated to obtain the desired product as a white solid (28 g). ¹H NMR (CDCl₃, 400 MHz): δ=8.79 (s, 1H), 8.20 (s, 1H), 8.16 (d, J=1.8 Hz, 1H), 8.04-8.12 (m, 1H), 7.55 ppm (d, J=8.4 Hz, 1H)

### Step B: Preparation of (2S)-2-(3,5-dichlorophenyl)-1,1,1-trifluoro-but-3-yn-2-ol

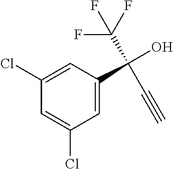 [SMILES: C#C[C@](O)(c1cc(Cl)cc(Cl)c1)C(F)(F)F]

To a solution of [(1S)-1-(3,5-dichlorophenyl)-1-(trifluoromethyl)prop-2-ynyl]butanoate (obtained as described in the preparative example P20 step C) (4.60 g) dissolved in dimethylsulfoxide (15 mL) was added sodium hydroxide 0.25M (220 mL) at room temperature. The mixture was stirred at room temperature for 17 hours then was heated to 60° C. for another 6.5 hours. Then more sodium hydroxide 2.0 M (13.6 mL) was added and the mixture was stirred at 60° C. for 16 hours. More sodium hydroxide (2.21 g) was added and the mixture was stirred at 60° C. for 5 hours. The solution was then cooled to 15° C. and acidified with HCl 37% to reach pH=3. The mixture was dissolved in diethylether, washed with water. The combined organic layers were dried over magnesium sulfate, filtered and evaporated to obtain a crude residue that was further dissolved in diethylether and washed with a sodium carbonate solution (1M). The combined organic layers were dried over magnesium sulfate, filtered and evaporated to obtain a bright yellow oil (3.60 g) as the titled compound. ¹H NMR (400 MHz, CDCl₃) 2.90 (s, 1H), 3.26 (s, OH), 7.45 (d, 1H), 7.55 (d, 2H); ¹⁹F-NMR (CDCl₃, 376.3 MHz): −80.40.

Chiral GC analysis:

GC was conducted on a Thermo Focus GC, with a column from Supelco Alpha DEX 120 fused

silica Capillary Column: 30m, diam: 0.25 mm, 0.25 um, H₂ flow 1. ml/min, temp injector: 220° C., FID Detector: temp detector: 300° C., method: start at 80° C., hold 2 min, 5.5° C./min until 220° C., hold 3 min, total time 30 min 2 isomers were detected: rt=23.37 min (29%) 24.32 min. (71%).

### Step C: Preparation of 5-[(3R)-3-(3,5-dichlorophenyl)-4,4,4-trifluoro-3-hydroxy-but-1-ynyl]-2-(1,2,4-triazol-1-yl)benzonitrile

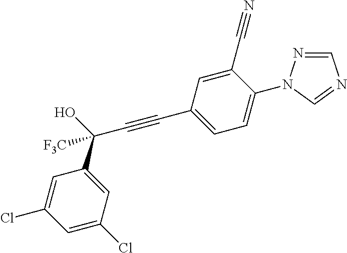 [SMILES: N#Cc1cc(C#C[C@](O)(c2cc(Cl)cc(Cl)c2)C(F)(F)F)ccc1-n1cncn1]

To a solution of [(1S)-1-(3,5-dichlorophenyl)-1-(trifluoromethyl)prop-2-ynyl]butanoate (4.0 g, 15 mmol) and 5-iodo-2-(1,2,4-triazol-1-yl)benzonitrile (4.4 g, 15 mmol) in N,N-dimethylformamide (36 mL) is added successively at room temperature triethylamine (30 g, 41 mL, 300 mmol), cooper-(I)-iodide (1.1 g, 5.9 mmol) and dichlorobis(triphenylphosphine)palladate(II) (1.1 g, 1.5 mmol) under argon. The mixture was heated to 80° C. for 3 hours then the mixture was dissolved in ethyl acetate. The suspension was washed with a hydrochloric solution (1M) to reach ph=4. The combined organic layers were dried over magnesium sulfate, filtered and evaporated to give a residue that was suspended in dichloromethane. The suspension was filtered and the solid was washed with dichloromethane then dried under vacuo to give the titled compound as beige solid (1.25 g).

The mother liquors were further concentrated under vacuo and purified using a silica gel column (eluent: cyclohexane/EtOAc) giving the titled compound as pale brown solid (2.92 g). ¹H NMR (DMSO d₆, 400 MHz): δ=9.31 (br. s., 1H), 8.56 (s, 1H), 8.43 (d, J=1.8 Hz, 2H), 8.12 (dd, J=8.4, 1.8 Hz, 1H), 7.99 (d, J=8.8 Hz, 1H), 7.80 (t, J=1.8 Hz, 1H), 7.68-7.77 ppm (m, 2H)

### Step D: Preparation of 5-[(E,3S)-3-(3,5-dichlorophenyl)-4,4,4-trifluoro-3-hydroxy-but-1-enyl]-2-(1,2,4-triazol-1-yl)benzonitrile

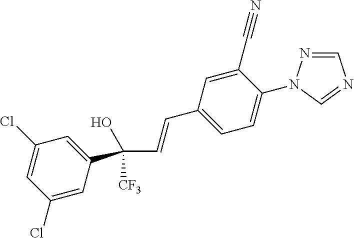 [SMILES: N#Cc1cc(/C=C/[C@](O)(c2cc(Cl)cc(Cl)c2)C(F)(F)F)ccc1-n1cncn1]

To a solution of 5-[(3R)-3-(3,5-dichlorophenyl)-4,4,4-trifluoro-3-hydroxy-but-1-ynyl]-2-(1,2,4-triazol-1-yl)benzonitrile (3.12 g) in 15.7 mL of toluene and 5.71 mL of THF cooled to −30° C., sodium bis(2-methoxyethoxy)aluminum hydride (65 mass % in Toluene) (2.56 mL) was added. The reaction was stirred at −30° C. for 3 h. The reaction mixture was carefully quenched first with acetone at −30° C. and then with NH₄Cl solution sat at −10° C. and extracted twice with ethyl acetate. The combined organic layers were dried (MgSO4), filtered and evaporated to give a yellow residue that was suspended in dichloromethane. The suspension was filtered and the solid was washed with dichloromethane then dried under vacuo to give the titled compound as yellow solid (1.49 g). The mother liquors were further concentrated under vacuo and purified using a silica gel column (eluent: cyclohexane/EtOAc) giving the titled compound (791 mg). ¹H-NMR (DMSO d₆, 400 MHz): δ=9.21 (s, 1H), 8.49 (d, J=1.8 Hz, 1H), 8.37 (s, 1H), 8.14 (dd, J=8.6, 2.0 Hz, 1H), 7.89 (d, J=8.4 Hz, 1H), 7.80 (d, J=1.8 Hz, 2H), 7.71 (t, J=1.8 Hz, 1H), 7.60 (s, 1H), 7.41 (d, J=15.8 Hz, 1H), 7.05 ppm (d, J=16.1 Hz, 1H)

### Step E: 5-[(2S)-2-(3,5-dichlorophenyl)-2-(trifluoromethyl)-3H-furan-4-yl]-2-(1,2,4-triazol-1-yl)benzonitrile (compound H1*)

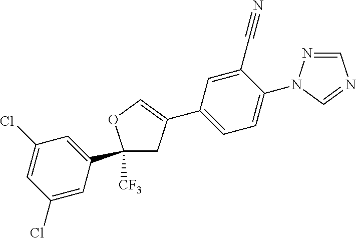 [SMILES: N#Cc1cc(C2=CO[C@@](c3cc(Cl)cc(Cl)c3)(C(F)(F)F)C2)ccc1-n1cncn1]

A homogeneous solution of [Rh(CO)₂acac] (0.0135 g), tris(2,4-ditert-butylphenyl)phosphite (0.336 g) and 5-[(E,3S)-3-(3,5-dichlorophenyl)-4,4,4-trifluoro-3-hydroxy-but-1-enyl]-2-(1,2,4-triazol-1-yl)benzonitrile (2.28 g) in tetrahydrofuran (20 mL) in a stainless steel autoclave was purged three times with hydrogen (5 bar), pressurized at 25 bar with H₂ followed by an additional 25 bar of CO (=50 bar CO/H₂ 1:1). The reaction was then heated at 100° C. and vigorously stirred for 70 h. The reaction was stopped by cooling the autoclave to room temperature, venting and purging with argon. The mixture was dissolved in ethyl acetate, washed with water. The combined organic layers were dried over magnesium sulfate, filtered and evaporated to obtain a crude residue which was purified using a silica gel column (eluent: cyclohexane/EtOAc) giving the hydrofomylated compound (2.14 g) as a beige foam that was used as such (mixture of diastereoisomers) in the next step.

The residue (2.47 g) was dissolved in xylenes (98 mL) and 4-methylbenzenesulfonic acid (1.10 g) was added. The mixture was heated to 120° C. for 15.5 hours. The mixture was then cooled to room temperature, and slowly poured on a cold saturated sodium carbonate solution. The mixture was diluted with ethyl acetate and washed twice with a saturated solution of sodium hydrogenocarbonate. The combined organic layers were dried over magnesium sulfate, filtered and evaporated to obtain a crude residue which was purified using a silica gel column (eluent: cyclohexane/EtOAc containing 1% NEt₃) to provide the titled compound (1.59 g) as a beige foam.

¹H-NMR (CDCl₃, 400 MHz): δ=8.76 (s, 1H), 8.19 (s, 1H), 7.68-7.78 (m, 1H), 7.56-7.67 (m, 2H), 7.49 (d, J=1.5 Hz, 2H), 7.40-7.47 (m, 1H), 7.13 (s, 1H), 3.78 (dd, J=15.2, 2.0 Hz, 1H), 3.36 ppm (d, J=15.4 Hz, 1H)

Chiral HPLC analysis:

Column: Daicel CHIRALPAK® IA, 3 μm, 0.46 cm×10 cm

Mobile phase: Heptan/iPrOH/DEA 70/30/0.1%

Flow rate: 1 ml/min

2 isomers were detected: rt=3.82 min (71.4%) 5.30 min. (28.6%). LC/MS Method A

| MS | ACQUITY SQD Mass Spectrometer from Waters (Single | | | | |
|---|---|---|---|---|---|
| | quadrupole mass spectrometer) | | | | |
| | Ionisation method: Electrospray | | | | |
| | Polarity: positive ions | | | | |
| | Capillary (kV) 3.00, Cone (V) 20.00, Extractor (V) 3.00, Source | | | | |
| | Temperature (° C.) 150, Desolvation Temperature (° C.) | | | | |
| | 400, Cone Gas Flow (L/Hr) 60, Desolvation Gas Flow (L/Hr) 700 | | | | |
| | Mass range: 100 to 800 Da | | | | |
| | DAD Wavelength range (nm): 210 to 400 | | | | |
| LC | Method Waters ACQUITY UPLC with the following HPLC gradient | | | | |
| | conditions (Solvent A: Water/Methanol 9:1, 0.1% formic | | | | |
| | acid and Solvent B: Acetonitrile, 0.1% formic acid) | | | | |
| | | Time (minutes) | A (%) | B (%) | Flow rate (ml/min) |
| | | 0 | 100 | 0 | 0.75 |
| | | 2.5 | 0 | 100 | 0.75 |
| | | 2.8 | 0 | 100 | 0.75 |
| | | 3.0 | 100 | 0 | 0.75 |

Type of column: Waters ACQUITY UPLC HSS T3; Column length:
30 mm; Internal diameter of column: 2.1 mm;
Particle Size: 1.8 micron; Temperature: 60° C.

### LC/MS: Method B

| MS | ZQ Mass Spectrometer from Waters (single quadrupole mass | | | |
|---|---|---|---|---|
| | spectrometer), ionization method: electrospray, polarity: negative | | | |
| | ionization, capillary (kV) 3.00, cone (V) 45.00, source temperature | | | |
| | (° C.) 100, desolvation temperature (° C.) 250, cone gas flow | | | |
| | (L/Hr) 50, desolvation gas flow (L/Hr) 400, mass range: 150 to 1000 Da. | | | |
| LC | HP 1100 HPLC from Agilent: solvent degasser, binary pump, | | | |
| | heated column compartment and diode-array detector. | | | |
| | Column: Phenomenex Gemini C18, length (mm) 30, internal diameter | | | |
| | (mm) 3, particle size (μm) 3, temperature (° C.) 60, DAD wavelength | | | |
| | range (nm): 200 to 500, solvent gradient: A = 0.05% v/v formic acid in | | | |
| | water and B = 0.04% v/v formic acid in acetonitrile/methanol (4:1). | | | |
| | Time (min) | A % | B % | Flow (ml/min) |
| | 0.0 | 95 | 5.0 | 1.7 |
| | 2.0 | 0.0 | 100 | 1.7 |
| | 2.8 | 0.0 | 100 | 1.7 |
| | 2.9 | 95 | 5.0 | 1.7 |
| | 3.1 | 95 | 5 | 1.7 |

TABLE A
(I-a)

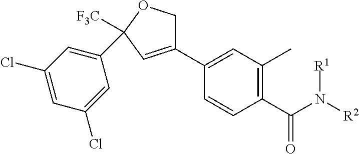

| Comp | | | | | |
|---|---|---|---|---|---|
| No. | R¹ | R² | RT (min) | MH⁺ | Method |
| A1 | H | (2,2,2-Trifluoro-ethylcarbamoyl)-methyl | 1.96 | 555 | A |
| A2 | H | Thietan-3-yl | 2.09 | 488 | A |
| A3 | H | 2,2,2-Trifluoro-ethyl | 2.10 | 498 | A |
| A4 | H | Ethyl | 2.02 | 444 | A |
| A5 | H | n-Butyl | 2.18 | 472 | A |
| A6 | H | 2-Methoxy-1-methyl-ethyl | 2.05 | 488 | A |
| A7 | H | (Tetrahydro-furan-2-yl)-methyl | 2.04 | 500 | A |
| A8 | H | Benzyl | 2.18 | 506 | A |
| A9 | H | 2-Fluoro-benzyl | 2.19 | 524 | A |
| A10 | H | 4-Methoxy-benzyl | 2.16 | 536 | A |
| A11 | H | 4-Methyl-thiazol-2-yl | 2.20 | 513 | A |
| A12 | H | 3-Methyl-thietan-3-yl | 2.19 | 502 | A |
| A13 | H | 1-Oxo-thietan-3-yl | 1.81 | 504 | A |
| A14 | H | Cyclobutyl | 2.14 | 470 | A |
| A15 | H | 1,1-Dioxo-thietan-3-yl | 1.91 | 520 | A |
| A16 | H | (S)-2-ethyl-isoxazolidin-3-one-4-yl | 2.11 | 527/529 | B |
| | | | | (M − H) | |
| A17 | H | (1-Methyl-1H-imidazol-4-yl)-methyl | 1.98 | 510 | A |
| A18 | H | (1H-Benzoimidazol-2-yl)-methyl | 1.66 | 546 | A |
| A19 | H | 3-Bromo-propyl | 2.07 | 536 | A |
| A20 | H | 3,3,3-Trifluoro-propyl | 2.09 | 512 | A |
| A21 | H | Dihydro-thiophen-2-one-3-yl | 2.01 | 516 | A |
| A22 | H | 6-Ethoxycarbonyl-cyclohex-3-enyl | 2.24 | 568 | A |
| A23 | H | 2-Benzo[1,3]dioxol-5-yl-ethyl | 2.14 | 564 | A |
| A24 | H | 2-Benzylsulfanyl-ethyl | 2.26 | 566 | A |
| A25 | H | 4-Methanesulfonyl-benzyl | 1.95 | 584 | A |
| A26 | H | N′,N′-Dimethylamino-ethyl | 1.49 | 487 | A |
| A27 | H | sec-Butyl | 2.13 | 472 | A |
| A28 | H | Butan-1-ol-2-yl | 1.90 | 488 | A |
| A29 | H | 2,2-Difluoro-ethyl | 2.01 | 480 | A |
| A30 | H | 1-Ethynyl-cyclohexyl | 2.26 | 522 | A |
| A31 | H | 2-[1,3]Dioxolan-2-yl-ethyl | 1.97 | 516 | A |
| A32 | H | 2-Methyl-cyclohexyl | 2.29 | 512 | A |
| A33 | H | 2-Morpholin-4-yl-ethyl | 1.44 | 529 | A |
| A34 | H | 3-Pyrrolidin-1-yl-propyl | 1.47 | 527 | A |
| A35 | H | (Pyrid-3-yl)-methyl | 1.67 | 507 | A |
| A36 | H | 3-Piperidin-1-yl-propyl | 1.50 | 541 | A |
| A37 | H | [3-(4-Chloro-phenyl)-isoxazol-5-yl]- | 2.27 | 607 | A |
| | | methyl | | | |
| A38 | H | 1-Phenyl-ethyl | 2.19 | 520 | A |
| A39 | H | Phenethyl | 2.19 | 520 | A |
| A40 | H | 1,2,2,6,6-Pentamethyl-piperidin-4-yl | 1.54 | 569 | A |
| A41 | H | 2-Thiophen-2-yl-ethyl | 2.17 | 526 | A |
| A42 | H | 2-Phenoxy-ethyl | 2.18 | 536 | A |
| A43 | H | 3-Chloro-benzyl | 2.23 | 540 | A |
| A44 | H | (2,3-Dihydro-benzo[1,4]dioxin-6-yl)- | 2.10 | 564 | A |
| | | methyl | | | |
| A45 | H | 2-Acetylamino-ethyl | 1.77 | 501 | A |
| A46 | H | 4-Pyrazol-1-yl-benzyl | 2.10 | 572 | A |
| A47 | H | 2-(1H-Indol-3-yl)-ethyl | 2.12 | 559 | A |
| A48 | H | 2-Trifluoromethyl-benzyl | 2.26 | 574 | A |
| A49 | H | 2-Methylsulfanyl-ethyl | 2.06 | 490 | A |
| A50 | H | 2-Piperidin-1-yl-benzyl | 1.95 | 589 | A |
| A51 | H | 4-Phenoxy-benzyl | 2.33 | 598 | A |
| A52 | H | (6-Chloro-pyridin-3-yl)-methyl | 2.06 | 541 | A |
| A53 | H | 1-Benzyl-pyrrolidin-3-yl | 1.62 | 575 | A |
| A54 | H | 2-(4-Benzyl-piperazin-1-yl)-ethyl | 1.61 | 618 | A |
| A55 | H | Furan-2-yl-methyl | 2.06 | 496 | A |
| A56 | H | 2-Chloro-phenyl | 2.32 | 526 | A |
| A57 | H | Quinolin-5-yl | 1.95 | 543 | A |
| A58 | H | 2,4-Dimethoxy-phenyl | 2.24 | 552 | A |
| A59 | H | 3-Fluoro-phenyl | 2.23 | 510 | A |
| A60 | H | 1H-Indazol-5-yl | 1.96 | 532 | A |
| A61 | H | 4-Pyrrol-1-yl-phenyl | 2.29 | 557 | A |
| A62 | H | 4-Piperidin-1-yl-phenyl | 1.86 | 575 | A |
| A63 | H | 2-Methylsulfanyl-phenyl | 2.33 | 538 | A |
| A64 | H | Benzothiazol-6-yl | 2.13 | 549 | A |
| A65 | H | 4-Methyl-2-oxo-2H-chromen-7-yl | 2.16 | 574 | A |
| A66 | H | 4-Dimethylsulfamoyl-phenyl | 2.14 | 599 | A |
| A67 | H | 2,5-Dimethyl-2H-pyrazol-3-yl | 1.98 | 510 | A |
| A68 | H | 5-Methylsulfanyl-1H-[1,2,4]triazol-3-yl | 2.15 | 529 | A |
| A69 | H | 4-Hydroxy-6-methyl-pyrimidin-2-yl | 1.94 | 524 | A |
| A70 | H | Quinolin-2-yl | 2.30 | 543 | A |
| A71 | H | 5-Methyl-3-phenyl-isoxazol-4-yl | 2.17 | 573 | A |
| A72 | H | 9H-Purin-6-yl | 1.84 | 534 | A |
| A73 | H | 5-Acetyl-4-methyl-thiazol-2-yl | 2.17 | 555 | A |
| A74 | H | 4-Methyl-benzothiazol-2-yl | 2.42 | 563 | A |
| A75 | H | 5-Methyl-[1,3,4]thiadiazol-2-yl | 2.05 | 514 | A |
| A76 | H | 4,6-Dimethyl-2H-pyrazolo[3,4-b]pyridin- | 1.98 | 561 | A |
| | | 3-yl | | | |
| A77 | H | 1-Oxo-thietan-3-yl | 1.78 | 504 | A |
| A78 | H | Thietan-3-yl-methyl | 2.07 | 502 | A |
| A79 | H | 3-(2,2,2-Trifluoro-ethoxyimino)- | 2.14 | 581 | A |
| | | cyclobutyl | | | |
| A80 | H | Thietan-2-yl-methyl | 2.09 | 502 | A |
| A81 | H | (1,1-Dioxo-thietan-2-yl)-methyl | 1.88 | 534 | A |
| A82 | H | 2-Thietan-3-yl-ethyl | 2.12 | 516 | A |
| A83 | H | 2-(1,1-Dioxo-thietan-3-yl)-ethyl | 1.87 | 548 | A |
| A84 | H | 3-Oxo-2-(2,2,2-trifluoro-ethyl)- | 2.03 | 583 | A |
| | | isoxazolidin-4-yl | | | |
| A85 | H | H | See NMR | See NMR | See NMR |
| A86 | H | 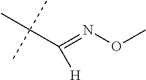 | See NMR | See NMR | See NMR |

TABLE B
(I-b)

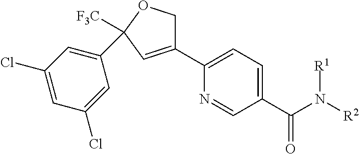

| Comp | | | | | |
|---|---|---|---|---|---|
| No. | R¹ | R² | RT (min) | MH⁺ | Method |
| B1 | H | 2,2,2-Trifluoro-ethyl | 1.99 | 485 | A |
| B2 | H | Ethyl | 1.87 | 431 | A |
| B3 | H | n-Butyl | 2.05 | 459 | A |
| B4 | H | 2-Methoxy-1-methyl-ethyl | 1.91 | 475 | A |
| B5 | H | (2,2,2-Trifluoro-ethylcarbamoyl)-methyl | 1.82 | 542 | A |
| B6 | H | 3,3,3-Trifluoro-propyl | 2.00 | 499 | A |
| B7 | H | sec-Butyl | 2.03 | 459 | A |
| B8 | H | (Tetrahydro-furan-2-yl)-methyl | 1.89 | 487 | A |
| B9 | H | Benzyl | 2.06 | 493 | A |
| B10 | H | 2-Fluoro-benzyl | 2.07 | 511 | A |
| B11 | H | 1-Phenyl-ethyl | 2.10 | 507 | A |
| B12 | H | 4-Methoxy-benzyl | 2.04 | 523 | A |
| B13 | H | 1,1-Dioxo-thietan-3-yl | 1.77 | 507 | A |
| B14 | H | (6-Chloro-pyrid-3-yl)-methyl | 1.96 | 528 | A |
| B15 | H | 3-Fluoro-phenyl | 2.15 | 497 | A |
| B16 | H | (Pyrid-2-yl)-methyl | 1.72 | 494 | A |
| B17 | H | 2,5-Dimethyl-2H-pyrazol-3-yl | 1.87 | 497 | A |
| B18 | H | 4-Methyl-thiazol-2-yl | 2.07 | 500 | A |
| B19 | H | 3-Methyl-thietan-3-yl | 2.05 | 489 | A |
| B20 | H | 1,1-Dimethyl-2-methylsulfanyl-ethyl | 2.14 | 505 | A |
| B21 | H | Thietan-3-yl | 1.96 | 475 | A |
| B22 | H | Bicyclo[2.2.1]hept-2-yl | 2.17 | 497 | A |
| B23 | H | Cyclobutyl | 2.00 | 457 | A |
| B24 | H | 1-Oxo-thietan-3-yl | 1.69 | 491 | A |

TABLE C
(I-c)

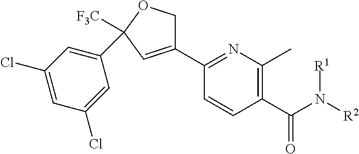

| Comp | | | | | |
|---|---|---|---|---|---|
| No. | R¹ | R² | RT (min) | MH⁺ | Method |
| C1 | H | 2,2,2-Trifluoro-ethyl | 2.07 | 499 | A |
| C2 | H | Ethyl | 1.94 | 445 | A |
| C3 | H | n-Butyl | 2.12 | 473 | A |
| C4 | H | 2-Methoxy-1-methyl-ethyl | 1.99 | 489 | A |
| C5 | H | (2,2,2-Trifluoro-ethylcarbamoyl)-methyl | 1.90 | 556 | A |
| C6 | H | 3,3,3-Trifluoro-propyl | 2.07 | 513 | A |
| C7 | H | sec-Butyl | 2.10 | 473 | A |
| C8 | H | (Tetrahydro-furan-2-yl)-methyl | 1.97 | 501 | A |
| C9 | H | Benzyl | 2.13 | 507 | A |
| C10 | H | 2-Fluoro-benzyl | 2.14 | 525 | A |
| C11 | H | 1-Phenyl-ethyl | 2.17 | 521 | A |
| C12 | H | 4-Methoxy-benzyl | 2.10 | 537 | A |
| C13 | H | 1,1-Dioxo-thietan-3-yl | 1.85 | 521 | A |
| C14 | H | (6-Chloro-pyrid-3-yl)-methyl | 2.04 | 542 | A |
| C15 | H | 3-Fluoro-phenyl | 2.22 | 511 | A |
| C16 | H | 2,5-Dimethyl-2H-pyrazol-3-yl | 1.96 | 511 | A |
| C17 | H | 3-Methyl-thietan-3-yl | 2.12 | 503 | A |
| C18 | H | 1,1-Dimethyl-2-methylsulfanyl-ethyl | 2.21 | 519 | A |
| C19 | H | Thietan-3-yl | 2.03 | 489 | A |
| C20 | H | Bicyclo[2.2.1]hept-2-yl | 2.23 | 511 | A |
| C21 | H | Cyclobutyl | 2.06 | 471 | A |
| C22 | H | 1-Oxo-thietan-3-yl | 1.76 | 505 | A |

TABLE D
(I-d)

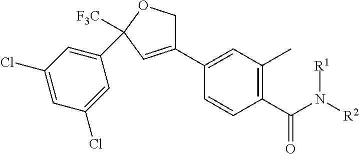

| Comp | | | | | |
|---|---|---|---|---|---|
| No. | R¹ | R² | RT (min) | MH⁺ | Method |
| D1 | H | 2,2,2-Trifluoro-ethyl | 2.10 | 498 | A |
| D2 | H | Ethyl | 1.99 | 444 | A |
| D3 | H | n-Butyl | 2.16 | 472 | A |
| D4 | H | 2-Methoxy-1-methyl-ethyl | 2.04 | 488 | A |
| D5 | H | (2,2,2-Trifluoro-ethylcarbamoyl)-methyl | 1.95 | 555 | A |
| D6 | H | 3,3,3-Trifluoro-propyl | 2.10 | 512 | A |
| D7 | H | sec-Butyl | 2.15 | 472 | A |
| D8 | H | (Tetrahydro-furan-2-yl)-methyl | 2.02 | 500 | A |
| D9 | H | Benzyl | 2.16 | 506 | A |
| D10 | H | 2-Fluoro-benzyl | 2.17 | 524 | A |
| D11 | H | 1-Phenyl-ethyl | 2.21 | 520 | A |
| D12 | H | 4-Methoxy-benzyl | 2.14 | 536 | A |
| D13 | H | (6-Chloro-pyrid-3-yl)-methyl | 2.07 | 541 | A |
| D14 | H | 3-Fluoro-phenyl | 2.29 | 510 | A |
| D15 | H | (Pyrid-2-yl)-methyl | 1.85 | 507 | A |
| D16 | H | 2,5-Dimethyl-2H-pyrazol-3-yl | 1.99 | 510 | A |
| D17 | H | 4-Methyl-thiazol-2-yl | 2.19 | 513 | A |
| D18 | H | 3-Methyl-thietan-3-yl | 2.16 | 502 | A |
| D19 | H | 1,1-Dimethyl-2-methylsulfanyl-ethyl | 2.27 | 518 | A |
| D20 | H | 1-Oxo-thietan-3-yl | 1.80 | 504 | A |
| D21 | H | Thietan-3-yl | 2.07 | 488 | A |
| D22 | H | Bicyclo[2.2.1]hept-2-yl | 2.29 | 510 | A |
| D23 | H | Cyclobutyl | 2.11 | 470 | A |
| D24 | H | 1,1-Dioxo-thietan-3-yl | 1.90 | 520 | A |

TABLE E
(I-e)

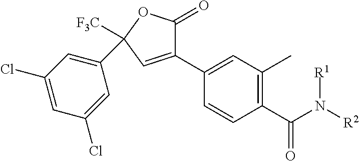

| Comp | | | | | |
|---|---|---|---|---|---|
| No. | R¹ | R² | RT (min) | MH⁺ | Method |
| E1 | H | 2,2,2-Trifluoro-ethyl | 2.01 | 512 | A |
| E2 | H | Ethyl | 1.91 | 458 | A |
| E3 | H | n-Butyl | 2.07 | 486 | A |
| E4 | H | 2-Methoxy-1-methyl-ethyl | 1.96 | 502 | A |
| E5 | H | (2,2,2-Trifluoro-ethylcarbamoyl)-methyl | 1.88 | 569 | A |
| E6 | H | 3,3,3-Trifluoro-propyl | 2.03 | 526 | A |
| E7 | H | (Tetrahydro-furan-2-yl)-methyl | 1.93 | 514 | A |
| E8 | H | Benzyl | 2.10 | 520 | A |
| E9 | H | 2-Fluoro-benzyl | 2.11 | 538 | A |
| E10 | H | 1-Phenyl-ethyl | 2.15 | 534 | A |
| E11 | H | 4-Methoxy-benzyl | 2.06 | 550 | A |
| E12 | H | (6-Chloro-pyrid-3-yl)-methyl | 2.00 | 555 | A |
| E13 | H | (Pyrid-2-yl)-methyl | 1.78 | 521 | A |
| E14 | H | 2,5-Dimethyl-2H-pyrazol-3-yl | 1.92 | 524 | A |
| E15 | H | 4-Methyl-thiazol-2-yl | 2.10 | 527 | A |
| E16 | H | 3-Methyl-thietan-3-yl | 2.10 | 516 | A |
| E17 | H | Thietan-3-yl | 2.02 | 502 | A |
| E18 | H | Cyclobutyl | 2.04 | 484 | A |
| E19 | H | 1-Oxo-thietan-3-yl | 1.73 | 518 | A |

TABLE F
(I-f)

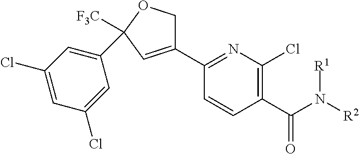

| Comp | | | | | |
|---|---|---|---|---|---|
| No. | R¹ | R² | RT (min) | MH⁺ | Method |
| F1 | H | (2,2,2-Trifluoro-ethylcarbamoyl)-methyl | See NMR | See NMR | See NMR |
| F2 | H | 1,1-Dioxo-thietan-3-yl | See NMR | See NMR | See NMR |

TABLE G
(I-g)

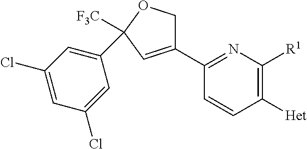

| Comp | | | | | |
|---|---|---|---|---|---|
| No. | R¹ | Het | RT (min) | MH⁺ | Method |
| G1 | CN | [1,2,4]triazol-1-yl | See NMR | See NMR | See NMR |
| G2 | CN | 4-Bromo-pyrazol-1-yl | See NMR | See NMR | See NMR |

The enantioenriched compounds can be obtained by chiral separation on preparative HPLC, which was conducted on compound D24 according to the following procedure:

Analytical HPLC method:

HPLC from Waters: allianceHT, 996 Waters UV/Visible Detector, 2795 Separation Module

Column: Daicel CHIRALPAK® IA-3, 0.46 cm×10 cm

Mobile phase: Heptan/2-Propanol/0.1DEA 70/30

Flow rate: 1 ml/min

Detection: UV 270 nm

Temperature: 25° C.

Preparative HPLC method:

Autopurification System from Waters: 2767 sample Manager, 2489 UV/Visible Detector, 2545 Quaternary Gradient Module.

Column: Daicel CHIRALPAK® IA 1.0 cm×25 cm

Mobile phase: Heptan/2-Propanol/0.1DEA 70/30

Flow rate: 5 ml/min

Detection: UV 270 nm

Temperature: 25° C.

Results:

From 101 mg crude material, 2 compounds could be isolated:

| | |
|---|---|
| First eluting enantiomer D24* | Second eluting enantiomer: D24** |
| Retention time (min) ~5.7 | Retention time (min) ~6.3 |
| Quantity (mg) 30.9 | Quantity (mg) 30.0 |
| Chemical purity (area % DAD) 100 | Chemical purity (area % DAD) 96 |
| Enantiomeric excess (%) >98 | Enantiomeric excess (%) >98 |

### Biological Examples of Racemic Mixtures

Spodoptera littoralis (Systemic) (Egyptian Cotton Leaf Worm)

Test compounds were applied by pipette into 24 well plates and mixed with agar. Salad seeds were placed on the agar and the multi well plate is closed by another plate which contains also agar. After 7 days the roots have absorbed the compound and the salad has grown into the lid plate. The salad leafs were now cut off into the lid plate. Spodoptera eggs were pipette through a plastic stencil on a humid gel blotting paper and the plate closed with it. The samples are checked for mortality, repellent effect, feeding behavior, and growth regulation 5 days after infestation. Application rate: 12.5 ppm

The following compound gave at least 80% control of Spodoptera littoralis: A1, A2, A13, A15, A16, C5, C13, A77, D24, F1, D1, D24, D5, D8, D15, D20, D21.

Spodoptera littoralis (Egyptian Cotton Leafworm):

Cotton leaf discs were placed on agar in a 24-well microtiter plate and sprayed with test solutions at an application rate of 200 ppm. After drying, the leaf discs were infested with 5 L1 larvae. The samples were checked for mortality, feeding behavior, and growth regulation 3 days after treatment (DAT).

The following compound gave at least 80% control of Spodoptera littoralis: A1, A2, A3, A4, A5, A6, A7, A8, A9, A10, A11, A12, A13, A14, A15, A16, C1, C2, C3, C4, C5, C6, C7, C8, C9, C10, C11, C12, C13, C14, C15, C17, C18, C19, C20, C21, C22, A86, G1, G2, A17, A19, A20, A21, A25, A26, A27, A28, A29, A31, A35, A38, A42, A43, A44, A49, A52, A53, A55, A57, A59, A60, A64, A67, A72, A77, A78, A79, A80, A81, A82, A83, A84, D24, F1, D1, D24, D3, D4, D5, D5*, D6, D7, D8, D9, D10, D11, D12, D13, D14, D15, D16, D17, D18, D19, D20, D21, D23, F2, E5, E6, E8, E13, E15, E19, D24, D25

Heliothis virescens (Tobacco Budworm):

Eggs (0-24 h old) were placed in 24-well microtiter plate on artificial diet and treated with test solutions at an application rate of 200 ppm (concentration in well 18 ppm) by pipetting. After an incubation period of 4 days, samples were checked for egg mortality, larval mortality, and growth regulation.

The following compound gave at least 80% control of Heliothis virescens: A1, A2, A3, A4, A5, A6, A7, A8, A9, A10, A11, A12, A13, A14, A15, A16, B5, B14, C1, C3, C5, C7, C8, C9, C10, C11, C12, C13, C14, C15, C16, C20, C21, C22, A86, G1, A17, A18, A19, A20, A21, A25, A27, A29, A35, A38, A39, A42, A44, A46, A49, A52, A54, A55, A56, A57, A58, A59, A60, A64, A66, A67, A69, A77, A78, A79, A80, A81, A82, A83, A84, D24, F1, D1, D24, D3, D4, D5, D5*, D6, D7, D8, D9, D10, D11, D12, D13, D14, D15, D16, D17, D18, D19, D20, D21, D23, F2, E5, D24, D25, H1*.

Plutella xylostella (Diamond Back Moth):

24-well microtiter plate (MTP) with artificial diet was treated with test solutions at an application rate of 200 ppm (concentration in well 18 ppm) by pipetting. After drying, the MTPs were infested with L2 larvae (7-12 per well). After an incubation period of 6 days, samples were checked for larval mortality and growth regulation.

The following compound gave at least 80% control of Plutella xylostella: A1, A2, A3, A4, A5, A6, A7, A8, A9, A10, A11, A12, A13, A14, A15, A16, B1, B7, B8, B9, B23, C1, C2, C3, C5, C6, C7, C9, C10, C11, C12, C13, C14, C15, C16, C17, C19, C20, C21, C22, A86, G1, A17, A18, A19, A20, A21, A25, A27, A29, A30, A31, A32, A35, A38, A39, A41, A44, A46, A49, A52, A55, A56, A57, A58, A59, A60, A63, A64, A67, A69, A77, A78, A79, A80, A81, A82, A83, A84, D24, F1, D1, D24, D3, D4, D5, D6, D7, D8, D9, D10, D11, D12, D13, D5*, D14, D15, D16, D17, D18, D19, D20, D21, D22, D23, F2, E3, E5, E6, E9, E11, E13, D24, D25, H1*.

Diabrotica balteata (Corn Root Worm):

A 24-well microtiter plate (MTP) with artificial diet was treated with test solutions at an application rate of 200 ppm (concentration in well 18 ppm) by pipetting. After drying, the MTPs were infested with L2 larvae (6-10 per well). After an incubation period of 5 days, samples were checked for larval mortality and growth regulation.

The following compound gave at least 80% control of Diabrotica balteata: A1, A2, A3, A4, A5, A6, A7, A8, A9, A10, A11, A12, A13, A14, A15, A16, B2, B6, B12, B19, C1, C2, C3, C5, C6, C7, C8, C9, C10, C11, C12, C13, C14, C17, C18, C19, C20, C21, C22, A86, G1, A17, A18, A19, A20, A21, A25, A27, A29, A30, A31, A32, A35, A38, A39, A41, A42, A43, A44, A46, A48, A49, A50, A52, A53, A54, A55, A56, A57, A58, A59, A60, A61, A63, A64, A65, A66, A67, A69, A75, A77, A78, A79, A80, A81, A82, A83, A84, D24, F1, D1, D24, D3, D4, D5, D5*, D6, D7, D8, D9, D10, D11, D12, D13, D14, D15, D16, D17, D18, D19, D20, D21, D22, D23, E5, E13, E15, E17, D24, D25, H1*.

Myzus persicae (Sachet) (Green Peach Aphid) Mixed Population

Test compounds were applied by pipette into 24 well plates and mixed with Sucrose solution. The plates were closed with a stretched Parafilm. A plastic stencil with 24 holes is placed onto the plate and infested pea seedlings were placed directly on the Parafilm. The infested plate is closed with a gel blotting paper and another plastic stencil and then turned upside down. 5 days after infestation the samples were checked on mortality. Application rate: 12.5 ppm.

The following compounds gave at least 80% control of Myzus persicae: A1, A2, A8, A13, A15, A16, C5, C13, C22, A25, A77, A78, A82, A83, A84, D24, F1, D24, D3, D4, D5, D5*, D6, D7, D8, D9, D10, D11, D12, D13, D14, D15, D16, D17, D18, D19, D20, D21, D23, D24, D25, H1*.

Thrips tabaci (Onion Thrips):

Sunflower leaf discs were placed on agar in a 24-well microtiter plate and sprayed with test solutions at an application rate of 200 ppm. After drying, the leaf discs were infested with an aphid population of mixed ages. After an incubation period of 7 days, samples were checked for mortality.

The following compounds gave at least 80% control of Thrips tabaci: A1, A2, A4, A5, A6, A7, A12, A13, A14, A15, A16, C2, C4, C5, C7, C13, C17, C19, C20, C21, C22, A86, A17, A19, A20, A27, A29, A30, A31, A59, A77, A78, A79, A82, A84, D24, F1, D1, D24, D3, D4, D5, D5*, D6, D8, D9, D10, D11, D12, D14, D15, D16, D18, D20, D21, D22, D23, E18, D24, D25, H1*.

Tetranychus urticae (Two-Spotted Spider Mite):

Bean leaf discs on agar in 24-well microtiter plates were sprayed with test solutions at an application rate of 200 ppm. After drying, the leaf discs are infested with mite populations of mixed ages. 8 days later, discs are checked for egg mortality, larval mortality, and adult mortality.

The following compound gave at least 80% control of Tetranychus urticae: A1, A2, A3, A4, A5, A6, A7, A8, A9, A10, A11, A12, A13, A14, A15, A16, B9, C2, C5, C13, C14, C17, C19, C22, A86, A17, A18, A19, A20, A25, A27, A28, A29, A30, A31, A35, A38, A40, A43, A45, A46, A49, A52, A56, A57, A58, A63, A77, A78, A79, A80, A81, A82, A83, A84, D24, F1, D24, D3, D4, D5, D5*, D6, D8, D9, D10, D11, D12, D13, D14, D15, D18, D19, D20, D21, D23, F2, E5, E16, D24, D25

### Biological Examples of Enantiomers

These examples illustrate the comparative insecticidal and acaricidal properties of compounds D24* and D24**. The tests were performed as follows:

Spodoptera littoralis (Systemic) (Egyptian Cotton Leaf Worm)

Test compounds were applied by pipette into 24 well plates and mixed with agar. Salad seeds were placed on the agar and the multi well plate is closed by another plate which contains also agar. After 7 days the roots have absorbed the compound and the salad has grown into the lid plate. The salad leafs were now cut off into the lid plate. Spodoptera eggs were pipette through a plastic stencil on a humid gel blotting paper and the plate closed with it. The samples are checked for mortality, repellent effect, feeding behavior, and growth regulation 5 days after infestation.

| Insects: Spodoptera littoralis | % Control at 200 ppm | % Control at 12.5 ppm |
|---|---|---|
| Compound D24* | 100 | 100 |
| Compound D24** | 100 | 0 |

Plutella xylostella (Diamond Back Moth):

24-well microtiter plate (MTP) with artificial diet was treated with test solutions at an application rate of 200 ppm (concentration in well 18 ppm) by pipetting. After drying, the MTPs were infested with L2 larvae (7-12 per well). After an incubation period of 6 days, samples were checked for larval mortality and growth regulation.

| Insects: Plutella xylostella | % Control at 200 ppm | % Control at 12.5 ppm |
|---|---|---|
| Compound D24* | 100 | 100 |
| Compound D24** | 100 | 0 |

Diabrotica balteata (Corn Root Worm):

A 24-well microtiter plate (MTP) with artificial diet was treated with test solutions at an application rate of 12.5 ppm (concentration in well 18 ppm) by pipetting. After drying, the MTP's were infested with L2 larvae (6-10 per well). After an incubation period of 5 days, samples were checked for larval mortality and growth regulation.

| Insects: Diabrotica balteata | % Control at 200 ppm |
|---|---|
| Compound D24* | 100 |
| Compound D24** | 50 |

Thrips tabaci (Onion Thrips):

Sunflower leaf discs were placed on agar in a 24-well microtiter plate and sprayed with test solutions at an application rate of 50 ppm. After drying, the leaf discs were infested with a thrip population of mixed ages. After an incubation period of 7 days, samples were checked for mortality.

| Insects: Thrips tabaci | % Control at 200 ppm |
|---|---|
| Compound D24* | 100 |
| Compound D24** | 0 |

Tetranychus urticae (Two-Spotted Spider Mite):

Bean leaf discs on agar in 24-well microtiter plates were sprayed with test solutions at an application rate of 200 ppm. After drying, the leaf discs are infested with mite populations of mixed ages. 8 days later, discs are checked for egg mortality, larval mortality, and adult mortality.

| Insects: Tetranychus urticae | % Control at 200 ppm |
|---|---|
| Compound D24* | 100 |
| Compound D24** | 0 |

1. A compound of formula (IA) 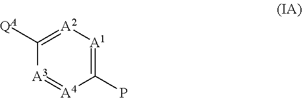
wherein
Q^{A} is Q^{A}1 or Q^{A}2 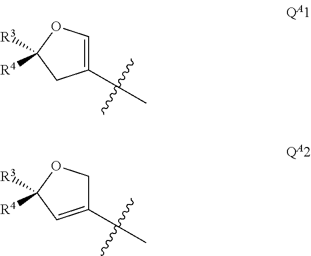
P is P1, heterocyclyl or heterocyclyl substituted by one to five Z; 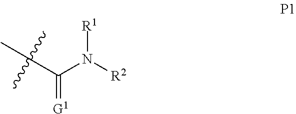
A¹, A², A³ and A⁴ are independently of each other C—H, C—R⁵, or nitrogen;
G¹ is oxygen or sulfur;
R¹ is hydrogen, C₁-C₈alkyl, C₁-C₈alkoxy-, C₁-C₈alkylcarbonyl-, or C₁-C₈alkoxycarbonyl-;
R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, C₁-C₆alkyl-O—N═CH—, C₁-C₆haloalkyl-O—N═CH—,
R³ is C₁-C₈haloalkyl;
R⁴ is aryl or aryl substituted by one to five R⁹, or heteroaryl or heteroaryl substituted by one to five R⁹;
each R⁵ is independently halogen, cyano, nitro, C₁-C₈alkyl, C₁-C₈haloalkyl, C₁-C₈alkenyl, C₁-C₈haloalkenyl, C₁-C₈alkynyl, C₁-C₈haloalkynyl, C₃-C₁₀cycloalkyl, C₁-C₈alkoxy-, C₁-C₈haloalkoxy-, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;
each R⁶ is independently halogen, cyano, nitro, hydroxy, amino, C₁-C₈alkylamino, (C₁-C₈alkyl)₂-amino, C₁-C₈alkylcarbonylamino, C₁-C₈haloalkylcarbonylamino, C₁-C₈alkoxy-, C₁-C₈haloalkoxy-, aryloxy or aryloxy substituted by one to five R¹⁰, aryloxy-C₁-C₄alkylene or aryloxy-C₁-C₄alkylene wherein the aryl moiety is substituted by one to five R¹⁰, C₁-C₈alkylcarbonyl-, C₁-C₈alkoxycarbonyl-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, aryl-C₁-C₄alkylthio- or aryl-C₁-C₄alkylthio- wherein the aryl moiety is substituted by one to five R¹⁰;
each R⁷ is independently halogen, C₁-C₈alkyl, C₁-C₈alkenyl, C₁-C₈alkynyl, C₁-C₈alkyl-O—N═, C₁-C₈haloalkyl-O—N═; C₁-C₈alkoxy, C₁-C₈akoxycarbonyl;
each R⁸ is independently halogen, cyano, nitro, oxo, C₁-C₈alkyl, C₁-C₈haloalkyl, C₁-C₈cyanoalkyl, C₂-C₈alkenyl, C₂-C₈haloalkenyl, C₂-C₈alkynyl, C₂-C₈haloalkynyl, C₃-C₁₀cycloalkyl, C₃-C₁₀cycloalkyl-C₁-C₄alkylene, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylaminosulfonyl, (C₁-C₈alkyl)₂-aminosulfonyl-, C₁-C₈alkylcarbonyl-, C₁-C₈alkoxycarbonyl-, aryl or aryl substituted by one to five R¹⁰, heterocyclyl or heterocyclyl substituted by one to five R¹⁰, aryl-C₁-C₄alkylene or aryl-C₁-C₄alkylene wherein the aryl moiety is substituted by one to five R¹⁰, heterocyclyl-C₁-C₄alkylene or heterocyclyl-C₁-C₄alkylene wherein the heterocyclyl moiety is substituted by one to five R¹⁰, aryloxy or aryloxy substituted by one to five R¹⁰, aryloxy-C₁-C₄alkylene or aryloxy-C₁-C₄alkylene wherein the aryl moiety is substituted by one to five R¹⁰;
each R⁹ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylcarbonyl-, C₁-C₈alkoxycarbonyl-, aryl or aryl substituted by one to five R¹⁰, or heterocyclyl or heterocyclyl substituted by one to five R¹⁰;
each R¹⁰ is independently halogen, cyano, nitro, C₁-C₄alkyl, C₁-C₄haloalkyl, C₁-C₄alkoxy-, or C₁-C₄haloalkoxy-;
each Z is independently halogen, C₁-C₁₂alkyl or C₁-C₁₂alkyl substituted by one to five R⁶, nitro, C₁-C₁₂alkoxy or C₁-C₁₂alkoxy substituted by one to five R⁶, cyano, C₁-C₁₂alkylsulfonyl, C₁-C₁₂haloalkylsulfinyl, C₁-C₁₂haloalkylsulfonyl, hydroxyl or thiol;
or a salt or N-oxide thereof.

2. A compound according to claim 1 wherein A¹ is C—R⁵, A² is C—H, A³ is C—H or nitrogen and A⁴ is C—H or nitrogen.

3. A compound according to claim 1 wherein G¹ is oxygen.

4. A compound according to claim 1, wherein P is P1 or a heterocycle selected from H1 to H9 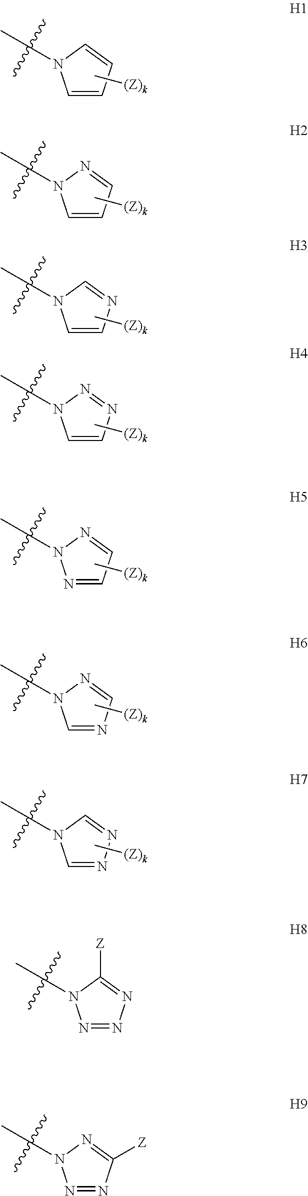
k is 0, 1 or 2.

5. A compound according to claim 1 wherein R¹ is hydrogen, methyl, ethyl, methylcarbonyl-, or methoxycarbonyl-.

6. A compound according to claim 1 wherein R² is C₁-C₈alkyl or C₁-C₈alkyl substituted by one to five R⁶, C₃-C₁₀cycloalkyl or C₃-C₁₀cycloalkyl substituted by one to five R⁷, aryl-C₁-C₄alkylene- or aryl-C₁-C₄alkylene- wherein the aryl moiety is substituted by one to five R⁸, heterocyclyl-C₁-C₄alkylene- or heterocyclyl-C₁-C₄alkylene- wherein the heterocyclyl moiety is substituted by one to five R⁸, aryl or aryl substituted by one to five R⁸, heterocyclyl or heterocyclyl substituted by one to five R⁸, C₁-C₈alkylaminocarbonyl-C₁-C₄ alkylene, C₁-C₈haloalkylaminocarbonyl-C₁-C₄ alkylene, C₃-C₈cycloalkyl-aminocarbonyl-C₁-C₄ alkylene, or group A 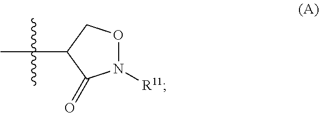
R¹¹ is hydrogen, methyl, ethyl, propyl, isopropyl, butyl, cyclopropyl, cyclopropyl-methyl, cyclobutyl, cyclobutyl-methyl, oxetanyl, thietanyl, trifluoroethyl, difluoroethyl, allyl, propargyl, cyanomethyl, benzyl, benzyl substituted by one to three R¹², or R¹¹ is pyridyl-methyl- or pyridyl-methyl- substituted by one to three R¹²;
each R¹² is independently fluoro, chloro, bromo, trifluoromethyl, trifluoromethoxy, cyano or methoxy;
wherein each aryl group is a phenyl group and each heterocyclyl group is independently selected from pyridyl, pyridazinyl, pyrimidinyl, pyrazinyl, pyrrolyl, pyrazolyl, imidazolyl, triazolyl, tetrahydrothiophenyl, tetrazolyl, furanyl, thiophenyl, oxazolyl, isoxazolyl, oxadiazolyl, thiazolyl, isothiazolyl, thiadiazolyl, quinolinyl, cinnolinyl, quinoxalinyl, indolyl, indazolyl, benzimidazolyl, benzothiophenyl, benzothiazolyl, oxetanyl, thietanyl, oxo-thietanyl, dioxo-thietanyl, pyrrolidinyl, tetrahydrofuranyl, [1,3]dioxolanyl, piperidinyl, piperazinyl, [1,4]dioxanyl, and morpholinyl, 2,3-dihydro-benzofuranyl, benzo[1,3]dioxolanyl, and 2,3-dihydro-benzo-[1,4]dioxinyl.

7. A compound according to claim 6 wherein R² is C₁-C₈alkyl or C₁-C₈alkyl substituted by one to five R⁶, C₃-C₁₀cycloalkyl or C₃-C₁₀cycloalkyl substituted by one to five R⁷, phenyl-C₁-C₄alkylene- or phenyl-C₁-C₄alkylene- wherein the phenyl moiety is substituted by one to five R⁸, pyridyl-C₁-C₄alkylene- or pyridyl-C₁-C₄alkylene- wherein the pyridyl moiety is substituted by one to four R⁸, oxetanyl or oxetanyl substituted by one to five R⁸, thietanyl-C₁-C₄ alkylene or thietanyl-C₁-C₄ alkylene wherein the thietanyl moiety is substituted by one to five R⁸, oxo-thietanyl-C₁-C₄ alkylene or oxo-thietanyl-C₁-C₄ alkylene wherein the oxo-thietanyl moiety is substituted by one to five R⁸, dioxo-thietanyl-C₁-C₄ alkylene or dioxo-thietanyl-C₁-C₄ alkylene wherein the dioxo-thietanyl moiety is substituted by one to five R⁸, thietanyl or thietanyl substituted by one to five R⁸, oxo-thietanyl or oxo-thietanyl substituted by one to five R⁸, dioxo-thietanyl or dioxo-thietanyl substituted by one to five R⁸, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene or group A

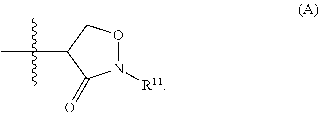

8. A compound according to claim 1 wherein R³ is chlorodifluoromethyl or trifluoromethyl.

9. A compound according to claim 1 wherein R⁴ is aryl or aryl substituted by one to five R⁹.

10. A compound according to claim 1, wherein
P is P1;
Q^{A} is Q^{A}1 or Q^{A}2;
A¹ is C—R⁵, A² is C—H, A³ is C—H and A⁴ is C—H;
G¹ is oxygen;
R¹ is hydrogen;
R² is C₁-C₈alkyl or C₁-C₈alkyl substituted by one to three halogen atoms, C₃-C₁₀cycloalkyl or C₃-C₁₀cycloalkyl substituted by one or two groups independently selected from fluoro and methyl, phenyl-C₁-C₄alkylene- or phenyl-C₁-C₄alkylene- wherein the phenyl moiety is substituted by one to five R⁸, pyridyl-C₁-C₄alkylene- or pyridyl-C₁-C₄alkylene- wherein the pyridyl moiety is substituted by one to four R⁸, thietanyl, oxo-thietanyl, dioxo-thietanyl, C₁-C₄alkylaminocarbonyl-C₁-C₄ alkylene, C₁-C₄haloalkylaminocarbonyl-C₁-C₄ alkylene, C₃-C₆cycloalkyl-aminocarbonyl-C₁-C₄ alkylene, or group A 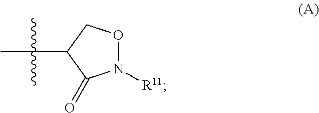
R³ is trifluoromethyl;
R⁴ is 3,5-dichloro-phenyl;
R⁵ is methyl;
each R⁸ is independently bromo, chloro, fluoro, cyano or methyl;
R¹¹ is methyl, ethyl or trifluoroethyl.

11. A compound of formula (IIA) 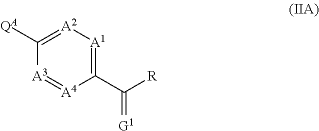
wherein Q^{A}, A¹, A², A³, and A⁴ are as defined for a compound of formula (IA) in claim 1, G¹ is oxygen, and R is hydroxy, C₁-C₁₅alkoxy or halogen; or a salt or N-oxide thereof;
or a compound of formula (IIIA) 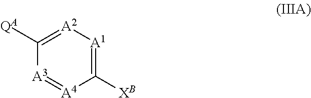
wherein Q^{A}, A¹, A², A³, A⁴ are as defined for a compound of formula (IA) in claim 1 and X^{B} is a leaving group; or a salt or N-oxide thereof;
or a compound of formula (XXVA) 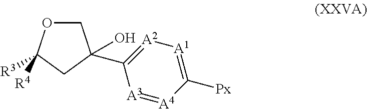
wherein A¹, A², A³, A⁴, R³ and R⁴ are as defined for a compound of formula (IA) in claim 1, Px is P as defined in claim 1, a leaving group, or C(O)R wherein R is halogen, OH or C₁-C₁₅alkoxy, or a salt or N-oxide thereof;
or a compound of formula (XXIIIA) 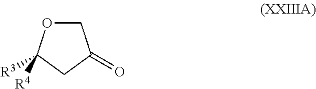
wherein R³ and R⁴ are as defined for a compound of formula (IA) in claim 1, or a salt or N-oxide thereof;
or a compound of formula (XXVIA) 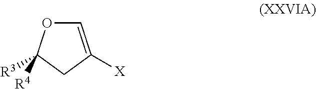
wherein R³ and R⁴ are as defined for a compound of formula (IA) in claim 1, and X is a tin derivative, a boron derivative, a leaving group, or C(O)R wherein R is halogen, OH or C₁-C₁₅alkoxy, or a salt or N-oxide thereof;
or a compound of formula (XXVIIA) 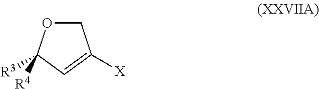
wherein R³ and R⁴ are as defined for a compound of formula (IA) in claim 1 and X is a tin derivative, a boron derivative, a leaving group, or C(O)R wherein R is halogen, OH or C₁-C₁₅alkoxy, or a salt or N-oxide thereof;
or a compound of formula (XXVIA′) 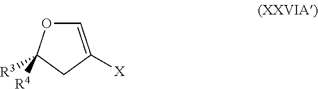
wherein R³ and R⁴ are as defined for the compounds of formula (IA) in claim 1 and X is Cl, Br or I, or a salt or N-oxide thereof;
or a compound of formula (XXVIIA′) 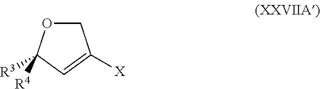
wherein R³ and R⁴ and as defined for the compound of formula (IA) in claim 1 and X is Cl, Br or I, or a salt or N-oxide thereof;
or a compound of formula (XXXVIA) 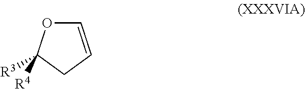
wherein R³ and R⁴ are as defined for a compound of formula (IA) in claim 1, or a salt or N-oxide thereof;
or a compound of formula (XXXVIIA) 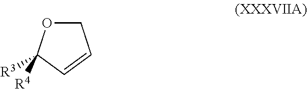
wherein R³ and R⁴ are as defined for a compound of formula (IA) in claim 1, or a salt or N-oxide thereof;
or a compound of formula (XXXVIIIA) 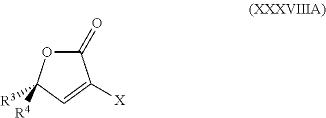
wherein R³ and R⁴ are as defined for a compound of formula (IA) in claim 1, and X is a tin derivative, a boron derivative, a leaving group, or C(O)R wherein R is halogen, OH or C₁-C₁₅alkoxy, or a salt or N-oxide thereof;
or a compound of formula (LA) 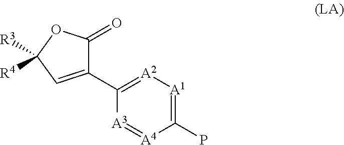
wherein R³, R⁴, A¹, A², A³, A⁴ and P are as defined for the compound of formula (IA) in claim 1, or a salt or N-oxide thereof; or
or a compound of formula (LXA) 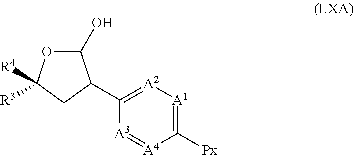
wherein R³, R⁴, A¹, A², A³, A⁴ are as defined for the compound of formula (IA) in claim 1 and Px is P as defined in claim 1, a leaving group, or C(O)R wherein R is halogen, OH or C₁-C₁₅alkoxy, or a salt or N-oxide thereof;
or a compound of formula (LIXA) 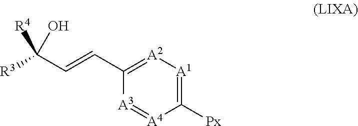
wherein R³, R⁴, A¹, A², A³, A⁴ are as defined for the compound of formula (IA) in claim 1 and Px is P as defined in claim 1, a leaving group, or C(O)R wherein R is halogen, OH or C₁-C₁₅alkoxy, or a salt or N-oxide thereof, or a salt or N-oxide thereof;
or a compound of formula (LIIA) 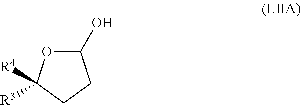
wherein R³ and R⁴ are as defined for the compound of formula (IA) in claim 1, or a salt or N-oxide thereof;
or a compound of formula (LIIIA) 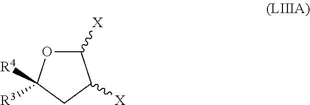
wherein R³ and R⁴ are as defined for the compound of formula (IA) in claim 1, and each X independently represents CI, Br or I, or a salt or N-oxide thereof;
or a compound of formula (LVA) 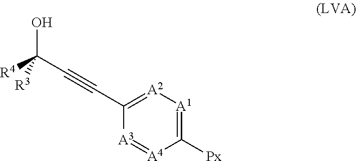
wherein R³, R⁴, A¹, A², A³ and A⁴ are as defined for the compound of formula (IA) in claim 1 and Px is P as defined in claim 1, a leaving group, or C(O)R wherein R is halogen, OH or C₁-C₁₅alkoxy, or a salt or N-oxide thereof;
or a compound of formula (LVIA) 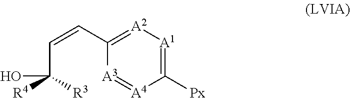
wherein R³, R⁴, A¹, A², A³ and A⁴ are as defined for the compound of formula (IA) in claim 1 and Px is P as defined in claim 1, a leaving group, or C(O)R wherein R is halogen, OH or C₁-C₁₅alkoxy, or a salt or N-oxide thereof.

12. A mixture comprising a compound of formula (IA) as defined in claim 1 and a compound of formula IB 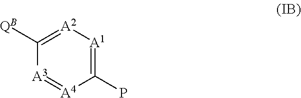
wherein Q^{B} is Q^{B}1 or Q^{B}2 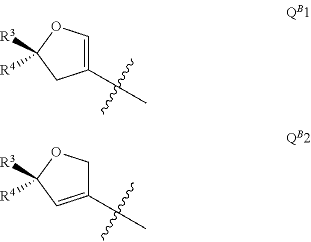
wherein A¹, A², A³, A⁴, P, R³ and R⁴ are as defined for the compound of formula (IA) in claim 1; and wherein the mixture is enriched for the compound of formula (IA).

13. (canceled)

14. A method of controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (IA) as defined in claim 1.

15. An insecticidal, acaricidal, nematicidal or molluscicidal composition comprising an insecticidally, acaricidally, nematicidally or molluscicidally effective amount of a compound of formula (IA) as defined in claim 1.

16. An insecticidal, acaricidal, nematicidal or molluscicidal composition according to claim 15 comprising at least one additional compound having biological activity.

17. A combination product comprising a pesticidally effective amount of a component A and a pesticidally effective amount of component B, wherein component A is a compound of formula (IA) as defined in claim 1, and compound B is imidacloprid, enrofloxacin, praziquantel, pyrantel embonate, febantel, penethamate, moloxicam, cefalexin, kanamycin, pimobendan, clenbuterol, fipronil, ivermectin, omeprazole, tiamulin, benazepril, milbemycin, cyromazine, thiamethoxam, pyriprole, deltamethrin, cefquinome, florfenicol, buserelin, cefovecin, tulathromycin, ceftiour, selamectin, carprofen, metaflumizone, moxidectin, methoprene (including S-methoprene), clorsulon, pyrantel, amitraz, triclabendazole, avermectin, abamectin, emamectin, eprinomectin, doramectin, selamectin, nemadectin, albendazole, cambendazole, fenbendazole, flubendazole, mebendazole, oxfendazole, oxibendazole, parbendazole, tetramisole, levamisole, pyrantel pamoate, oxantel, morantel, triclabendazole, epsiprantel, fipronil, lufenuron, ecdysone or tebufenozide.

18. A process for the preparation of a compound of formula (XIX) 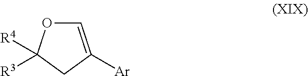
wherein
Ar is group A 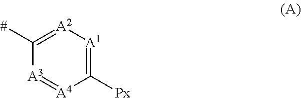
R³, R⁴, A¹, A², A³, A⁴ are as defined for the compound of formula (IA) in claim 1, and Px is P as defined in claim 1, a leaving group, or C(O)R wherein R is halogen, OH or C₁-C₁₅alkoxy, or a salt or N-oxide thereof;
comprising dehydrating a compound of formula (LX) 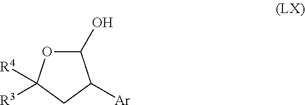
wherein R³, R⁴ and Ar are as defined for the compound of formula (XIX);
with a suitable acidic catalyst or a suitable activation agent and a suitable base.

19. (canceled)

20. A compound of formula (LX) 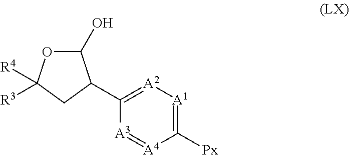
wherein R³, R⁴, A¹, A², A³, A⁴ are as defined for the compound of formula (IA) in claim 1 and Px is P as defined in claim 1, a leaving group, or C(O)R wherein R is halogen, OH or C₁-C₁₅alkoxy, or a salt or N-oxide thereof; or
a compound of formula (LIX) 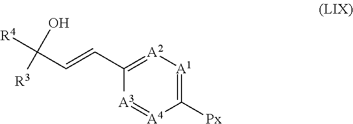
wherein R³, R⁴, A¹, A², A³, A⁴ are as defined for the compound of formula (IA) in claim 1 and Px is P as defined in claim 1, a leaving group, or C(O)R wherein R is halogen, OH or C₁-C₁₅alkoxy, or a salt or N-oxide thereof; or
a compound of formula (LII) 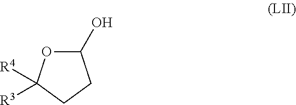
wherein R³ and R⁴ are as defined for the compound of formula (IA) in claim 1, or a salt or N-oxide thereof; or
a compound of formula (LIII) 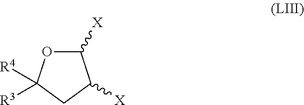
wherein R³ and R⁴ are as defined for the compound of formula (IA) in claim 1, and each X independently represents Cl, Br or I, or a salt or N-oxide thereof; or
a compound of formula (XXVI) 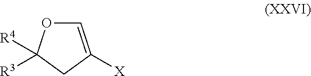
wherein R³ and R⁴ are as defined for the compound of formula (IA) in claim 1 and X is Cl, Br or I; or a salt or N-oxide thereof; or
a compound of formula (XXXVI) 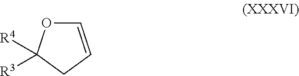
wherein R³ and R⁴ are as defined for the compound of formula (IA) in claim 1, or a salt of N-oxide thereof; or
a compound of formula (LI) 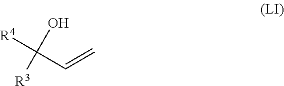
wherein R³ and R⁴ are as defined for the compound of formula (IA) in claim 1; or a salt or N-oxide thereof; or
a compound of formula (LV) 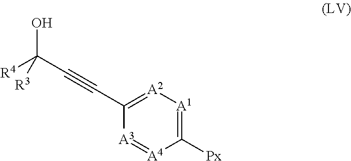
wherein R³, R⁴, A¹, A², A³ and A⁴ are as defined for the compound of formula (IA) and Px is P as defined in claim 1, a leaving group, or C(O)R wherein R is halogen, OH or C₁-C₁₅alkoxy, or a salt or N-oxide thereof; or
a compound of formula (LVI) 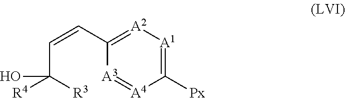
wherein R³, R⁴, A¹, A², A³ and A⁴ are as defined for the compound of formula (IA) and Px is P as defined in claim 1, a leaving group, or C(O)R wherein R is halogen, OH or C₁-C₁₅alkoxy, or a salt or N-oxide thereof.
